(19) United States
(12) Patent Application Publication
(10) Pub. No.: US 2012/0260373 A1
(43) Pub. Date: Oct. 11, 2012

(54) TRANSGENIC PLANTS HAVING ENHANCED BIOMASS COMPOSITION

(76) Inventor: Nestor Apuya, Culver City, CA (US)

(21) Appl. No.: 13/500,581

(22) PCT Filed: Oct. 6, 2010
(86) PCT No.: PCT/US10/51657

§ 371 Date: Jun. 21, 2012

Related U.S. Application Data
(60) Provisional application No. 61/249,508, filed on Oct. 7, 2009.

(51) Int. Cl. A01H 5/00; C12N 5/10

(52) U.S. Cl. 800/306; 435/419; 800/298; 800/320.1; 800/312; 800/320.3; 800/314; 800/320.2; 800/322; 800/320

(57) ABSTRACT

Methods and materials for modulating biomass composition in plants are disclosed. For example, nucleic acids encoding biomass-modulating polypeptides are disclosed as well as methods for using such nucleic acids to transform plant cells. Also disclosed are plants having enhanced biomass composition and plant products produced from plants having enhanced biomass composition.

### CROSS-REFERENCE TO RELATED APPLICATION

This application claims the benefit of U.S. Provisional Application No. 61/249,508, filed Oct. 7, 2009. The disclosure of the foregoing application is hereby incorporated by reference in its entirety.

### STATEMENT AS TO FEDERALLY SPONSORED RESEARCH

This invention was made with government support under USDA Biomass Research and Development Initiative Grant 68-3A75-7-601. The government has certain rights in the invention.

### INCORPORATION-BY-REFERENCE OF SEQUENCE LISTING OR TABLE

The material in the accompanying sequence listing is hereby incorporated by reference into this application. The accompanying file, 9-24-09_sequence_listing.txt was created on Sep. 24, 2009 and is 8,553 KB. The file can be accessed using Microsoft Word on a computer that uses Windows OS.

### TECHNICAL FIELD

This document relates to methods and materials involved in modulating biomass levels in plants. For example, this document provides plants having enhanced biomass composition as well as materials and methods for making plants and plant products having enhanced biomass composition.

### BACKGROUND

The present invention relates to methods of increasing biomass in plants and plants generated thereby. Plants having increased and/or improved biomass are useful for agriculture, horticulture, biomass to energy conversion, paper production, plant product production, and other industries. In particular, there is a need for increases in biomass for dedicated energy crops such as Panicum virgatum L. (switchgrass), Miscanthus×gigantus (miscanthus), Sorghum sp., and Saccharum sp. (sugar cane). Throughout human history, access to plant biomass for both food and fuel has been essential to maintaining and increasing population levels. Scientists are continually striving to improve biomass in agricultural crops. The large amount of research related to increasing plant biomass, particularly for dedicated energy crops, indicates the level of importance placed on providing sustainable sources of energy for the population. The urgency of developing sustainable and stable sources of plant biomass for energy is underscored by current events, such as rising oil prices. The amount of biomass produced by plants is a quantitative trait affected by a number of biochemical pathways. There is a need for molecular genetic approaches to more rapidly produce plants having increased biomass. There is also a need to produce plant species that grow more efficiently and produce more biomass in various geographic and/or climatic environments. It would be desirable for such approaches to be applicable to multiple plant species (Zhang et al., Plant Physiol. 135: 615-621 (2004)). Despite some progress in molecular genetic approaches, there is also a need to identify specific genes and/or sequences that can be used to effectively increase biomass in plants.

Conversion of biomass into cellulosic ethanol biofuel is impeded by lignin, a major structural component of secondarily thickened cell walls of tissues. As the second most abundant polymer on earth, exceeded only by cellulose, lignin is a major carbon sink in the biosphere, accounting for about 30% of the carbon sequestered into terrestrial plant material each year (Battle et al., Science, 287:2467 (2000)). Lignin is a major structural component of secondarily thickened cell walls of tissues with conducting and/or mechanical functions. Angiosperm lignin is composed of three main units named p-hydroxyphenyl (H), guaiacyl (G), and syringyl (S) units. These components originate from the polymerization of three monolignols, p-coumaryl, coniferyl, and sinapyl alcohols, respectively. The monolignols are synthesized from phenylalanine through successive deamination, reduction, hydroxylation, and methylation steps. The proportions of H, G, and S units in the cell wall vary according to plant species and tissue type.

Cellulosic ethanol, which exhibits a net energy content three times higher than corn ethanol, can be produced from a wide variety of cellulosic biomass feedstocks including agricultural plant wastes, plant wastes from industrial processes and energy crops grown specifically for fuel production. Cellulosic biomass is composed largely of cellulose, hemicellulose and lignin, with smaller amounts of proteins, lipids and ash, an undesirable remnant of combustion. Processing cellulosic biomass aims to extract fermentable sugars from the feedstock, which requires disruption of the hemicellulose/lignin sheath that surrounds the cellulose in plant material. Technological developments that increase the yield and drive down the production cost of cellulosic ethanol can help to reduce our oil dependency in a sustainable way. Given the role of lignin in the recalcitrance of biomass for conversion to biobased fuels, in addition to the many other roles of lignin, it is desirable to have the ability to produce plants with modulated levels of lignin.

As a major polymer of cell walls, lignin also has a direct impact on the characteristics of plants and plant products, such as wood. Highly lignified wood is durable and therefore a good raw material for many applications. Since lignin yields more energy when burned than cellulose, lignified wood is also an excellent fuel. The mechanical support provided by lignin prevents lodging, a problem in many agronomically important plants. On the other hand, lignin is detrimental to paper manufacture and must be removed from pulp before paper can be manufactured. This is costly both in terms of energy and the environment.

### SUMMARY

This document provides methods and materials related to plants having enhanced biomass composition. For example, this document provides transgenic plants and plant cells having modulated levels of biomass, nucleic acids used to generate transgenic plants and plant cells having modulated composition of biomass, methods for making plants having modulated composition of biomass, and methods for making plant cells that can be used to generate plants having modulated level of biomass components such as glucan, lignin, or ash. Such plants and plant cells can be grown to produce, for example, plants having enhanced biomass composition, e.g., increased dry weight, decreased ash content, increased or decreased lignin content, or increased glucan content. Plants having enhanced biomass composition may be useful to produce biomass for food and feed, which may benefit both humans and animals. Plants having enhanced biomass composition may be useful in converting such biomass to a liquid fuel (e.g., ethanol), or other chemicals, or may be useful as a thermochemical fuel.

Methods of producing a plant having increased biomass are provided herein. In one aspect, a method comprises growing a plant cell comprising an exogenous nucleic acid. The exogenous nucleic acid comprises a regulatory region operably linked to a nucleotide sequence encoding a polypeptide. The Hidden Markov Model (HMM) bit score of the amino acid sequence of the polypeptide is greater than about 915, 1149, 618, 324, 752, 866, 703, 862, 883, 393, 232, 815, 1251, 1066, 1398, 370, 328, 133, 265, 843, 93, 175, 2105, 1213, 1174, 1138, 1126, 1812, 824, 1240, 1078, 873, 1102, 754, 539, 657, 1925, 1104, 1347, 1096, 851, 761, 815, 1121, 681, 677, 146, 286, 493, or 316 using an HMM generated from the amino acid sequences depicted in one of FIG. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, respectively. The plant has a difference in the level of biomass as compared to the corresponding level of biomass of a control plant that does not comprise the exogenous nucleic acid.

In another aspect, a method comprises growing a plant cell comprising an exogenous nucleic acid. The exogenous nucleic acid comprises a regulatory region operably linked to a nucleotide sequence encoding a polypeptide having 80 percent or greater sequence identity to an amino acid sequence set forth in SEQ ID NOs: 2, 4, 5, 6, 8, 10, 12, 13, 15, 17, 18, 20, 22, 24, 25, 26, 27, 29, 30, 32, 34, 35, 37, 38, 39, 41, 43, 45, 47, 49, 51, 53, 55, 56, 57, 59, 60, 62, 63, 64, 65, 67, 69, 70, 71, 73, 74, 75, 77, 79, 80, 81, 82, 83, 84, 85, 86, 88, 90, 91, 92, 93, 94, 95, 96, 97, 98, 99, 100, 101, 102, 103, 104, 105, 106, 107, 108, 109, 110, 111, 112, 113, 115, 117, 119, 120, 121, 122, 123, 124, 126, 128, 129, 131, 133, 135, 137, 139, 140, 141, 142, 144, 146, 147, 148, 149, 150, 152, 154, 155, 157, 158, 160, 162, 163, 165, 167, 169, 171, 173, 175, 176, 178, 180, 182, 183, 185, 187, 189, 191, 193, 194, 195, 196, 197, 198, 199, 200, 201, 203, 205, 207, 208, 209, 211, 213, 214, 215, 217, 218, 219, 220, 222, 223, 224, 226, 228, 229, 231, 232, 233, 234, 235, 236, 237, 238, 239, 240, 242, 244, 245, 246, 247, 248, 249, 250, 251, 252, 254, 255, 256, 257, 258, 259, 260, 261, 262, 263, 264, 265, 266, 267, 268, 269, 270, 271, 272, 273, 274, 275, 276, 277, 278, 279, 281, 282, 284, 286, 287, 288, 289, 291, 293, 294, 295, 297, 299, 300, 302, 304, 306, 307, 308, 310, 311, 313, 315, 316, 317, 318, 319, 320, 322, 324, 326, 328, 330, 331, 333, 335, 337, 338, 340, 342, 343, 345, 346, 347, 349, 350, 351, 353, 354, 355, 357, 358, 360, 362, 363, 364, 365, 367, 369, 370, 371, 372, 373, 374, 375, 377, 378, 379, 380, 382, 383, 384, 385, 386, 387, 389, 390, 391, 393, 395, 396, 398, 400, 401, 403, 404, 406, 408, 409, 410, 411, 412, 414, 415, 417, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 456, 457, 459, 461, 462, 464, 466, 467, 468, 469, 470, 472, 474, 475, 477, 479, 480, 482, 483, 484, 485, 487, 488, 489, 490, 491, 492, 493, 494, 495, 497, 499, 501, 502, 503, 504, 506, 507, 509, 510, 511, 512, 514, 515, 516, 517, 518, 520, 522, 524, 526, 528, 530, 531, 532, 533, 534, 535, 536, 537, 538, 539, 540, 542, 543, 545, 546, 547, 548, 550, 551, 553, 554, 555, 557, 559, 560, 562, 563, 565, 566, 568, 570, 572, 573, 575, 576, 577, 579, 580, 581, 582, 583, 584, 585, 586, 587, 589, 590, 591, 592, 593, 595, 596, 597, 598, 599, 601, 602, 604, 605, 607, 609, 610, 611, 612, 613, 614, 615, 616, 617, 618, 619, 620, 621, 622, 623, 624, 625, 626, 627, 629, 631, 633, 634, 635, 636, 637, 638, 640, 642, 644, 646, 648, 650, 651, 653, 654, 655, 656, 657, 658, 659, 660, 662, 663, 664, 665, 666, 667, 669, 671, 672, 674, 676, 677, 679, 680, 682, 684, 685, 687, 689, 690, 691, 693, 694, 696, 697, 698, 700, 702, 704, 705, 706, 707, 709, 710, 712, 714, 715, 717, 718, 719, 720, 721, 722, 723, 725, 726, 727, 728, 730, 731, 732, 733, 734, 735, 737, 739, 741, 743, 744, 745, 746, 747, 749, 751, 752, 753, 755, 757, 759, 760, 762, 764, 766, 768, 770, 772, 773, 775, 776, 778, 780, 782, 784, 786, 788, 789, 791, 793, 794, 795, 796, 798, 800, 801, 802, 804, 805, 807, 809, 811, 813, 815, 817, 819, 820, 822, 823, 824, 825, 827, 828, 830, 832, 834, 836, 838, 840, 842, 843, 844, 846, 848, 849, 850, 851, 853, 855, 856, 857, 858, 860, 861, 863, 864, 866, 868, 870, 871, 873, 874, 876, 878, 880, 882, 883, 885, 887, 889, 890, 892, 893, 895, 896, 897, 899, 901, 902, 903, 905, 907, 908, 909, 910, 912, 914, 915, 916, 917, 919, 921, 923, 925, 926, 928, 930, 932, 934, 935, 936, 937, 939, 941, 943, 945, 947, 949, 950, 952, 953, 955, 957, 959, 960, 962, 964, 965, 967, 968, 970, 972, 974, 976, 978, 980, 981, 983, 985, 86, 87, 989, 991, 993, 995, 997, 998, 1000, 1002, 1003, 1005, 1007, 1008, 1009, 1011, 1013, 1014, 1016, 1018, 1020, 1022, 1023, 1024, 1025, 1026, 1027, 1029, 1031, 1033, 1034, 1036, 1038, 1039, 1041, 1042, 1043, 1044, 1045, 1046, 1048, 1049, 1050, 1051, 1052, 1054, 1056, 1058, 1060, 1061, 1062, 1064, 1066, 1067, 1069, 1070, 1071, 1072, 1074, 1076, 1077, 1078, 1079, 1081, 1083, 1085, 1087, 1088, 1090, 1092, 1093, 1095, 1096, 1097, 1099, 1101, 1102, 1104, 1105, 1107, 1108, 1110, 1111, 1112, 1113, 1114, 1115, 1117, 1119, 1120, 1121, 1122, 1124, 1126, 1128, 1130, 1132, 1133, 1135, 1137, 1139, 1141, 1142, 1144, 1146, 1148, 1150, 1152, 1154, 1156, 1157, 1158, 1160, 1162, 1164, 1166, 1168, 1169, 1171, 1173, 1175, 1176, 1177, 1179, 1180, 1181, 1183, 1185, 1187, 1188, 1190, 1192, 1193, 1194, 1196, 1198, 1200, 1201, 1203, 1205, 1207, 1209, 1210, 1212, 1214, 1216, 1218, 1219, 1221, 1222, 1224, 1226, 1228, 1230, 1232, 1234, 1235, 1236, 1238, 1240, 1241, 1243, 1245, 1247, 1249, 1251, 1252, 1253, 1255, 1257, 1259, 1261, 1263, 1265, 1266, 1268, 1270, 1272, 1274, 1276, 1278, 1279, 1281, 1283, 1285, 1287, 1289, 1291, 1293, 1294, 1296, 1297, 1299, 1300, 1302, 1304, 1306, 1308, 1309, 1310, 1312, 1313, 1314, 1316, 1318, 1320, 1322, 1324, 1325, 1326, 1328, 1329, 1330, 1332, 1333, 1334, 1336, 1338, 1339, 1340, 1341, 1342, 1343, 1344, 1345, 1347, 1349, 1350, 1352, 1353, 1355, 1357, 1358, 1359, 1360, 1361, 1363, 1364, 1366, 1367, 1369, 1370, 1371, 1372, 1373, 1375, 1376, 1377, 1379, 1380, 1381, 1382, 1384, 1386, 1388, 1389, 1391, 1393, 1395, 1397, 1399, 1401, 1402, 1403, 1405, 1407, 1409, 1410, 1412, 1414, 1416, 1418, 1420, 1421, 1422, 1424, 1426, 1428, 1429, 1431, 1433, 1435, 1437, 1439, 1440, 1441, 1443, 1445, 1446, 1448, 1450, 1452, 1454, 1456, 1457, 1459, 1461, 1463, 1465, 1467, 1469, 1471, 1473, 1475, 1477, 1479, 1481, 1482, 1483, 1485, 1486, 1488, 1489, 1490, 1492, 1493, 1495, 1497, 1498, 1500, 1502, 1503, 1505, 1506, 1507, 1508, 1509, 1511, 1512, 1514, 1515, 1516, 1518, 1520, 1522, 1523, 1524, 1525, 1526, 1528, 1529, 1531, 1533, 1534, 1535, 1537, 1538, 1540, 1542, 1544, 1546, 1547, 1549, 1550, 1552, 1554, 1555, 1557, 1558, 1560, 1562, 1563, 1564, 1565, 1568, 1569, 1571, 1572, 1574, 1575, 1577, 1578, 1580, 1581, 1583, 1584, 1586, 1588, 1589, 1591, 1593, 1594, 1595, 1596, 1598, 1599, 1600, 1602, 1603, 1604, 1605, 1607, 1609, 1611, 1612, 1613, 1615, 1616, 1618, 1619, 1621, 1622, 1624, 1626, 1628, 1629, 1630, 1632, 1634, 1636, 1637, 1639, 1640, 1642, 1644, 1646, 1647, 1648, 1649, 1650, 1652, 1654, 1656, 1657, 1658, 1660, 1661, 1662, 1664, 1666, 1668, 1669, 1670, 1671, 1672, 1674, 1676, 1677, 1679, 1680, 1681, 1682, 1683, 1684, 1686, 1688, 1689, 1691, 1693, 1695, 1697, 1699, 1700, 1701, 1702, 1704, 1705, 1706, 1707, 1708, 1710, 1711, 1712, 1713, 1714, 1715, 1717, 1719, 1721, 1723, 1724, 1725, 1727, 1729, 1731, 1732, 1734, 1735, 1737, 1739, 1741, 1743, 1745, 1746, 1747, 1749, 1751, 1753, 1755, 1757, 1758, 1759, 1761, 1762, 1764, 1765, 1766, 1767, 1769, 1770, 1772, 1773, 1774, 1776, 1777, 1779, 1781, 1783, 1784, 1786, 1788, 1790, 1792, 1794, 1795, 1797, 798, 1799, 1801, 1803, 1804, 1805, 1806, 1807, 1808, 1809, 1810, 1811, 1812, 1813, 1815, 1816, 1817, 1819, 1821, 1822, 1823, 1825, 1826, 1827, 1828, 1829, 1831, 1833, 1834, 1835, 1837, 1839, 1841, 1842, 1844, 1845, 1847, 1849, 1850, 1852, 1853, 1854, 1855, 1856, 1857, 1858, 1859, 1861, 1863, 1865, 1866, 1867, 1869, 1871, 1872, 1874, 1876, 1877, 1879, 1881, 1882, 1883, 1885, 1887, 1889, 1891, 1893, 1894, 1895, 1897, 1898, 1900, 1902, 1903, 1904, 1906, 1908, 1910, 1912, 1913, 1915, 1917, 1918, 1919, 1920, 1922, 1924, 1926, 1928, 1929, 1931, 1933, 1935, 1936, 1938, 1940, 1942, 1944, 1946, 1948, 1949, 1951, 1953, 1955, 1957, 1959, 1961, 1963, 1965, 1967, 1969, 1971, 1972, 1973, 1974, 1975, 1976, 1978, 1979, 1980, 1982, 1983, 1985, 1986, 1987, 1989, 1990, 1992, 1994, 1996, 1997, 1999, 2000, 2002, 2003, 2005, 2007, 2008, 2009, 2010, 2011, 2013, 2015, 2016, 2017, 2018, 2019, 2021, 2022, 2023, 2024, 2026, 2028, 2030, 2031, 2032, 2034, 2036, 2037, 2038, 2040, 2041, 2042, 2043, 2044, 2046, 2047, 2048, 2049, 2051, 2053, 2055, 2056, 2058, 2060, 2062, 2064, 2066, 2067, 2069, 2070, 2072, 2073, 2074, 2075, 2076, 2077, 2078, 2079, 2081, 2083, 2085, 2087, 2088, 2090, 2091, 2092, 2093, 2095, 2097, 2098, 2100, 2102, 2104, 2105, 2106, 2108, 2110, 2112, 2113, 2115, 2117, 2118, 2119, 2120, 2121, 2122, 2124, 2126, 2128, 2129, 2130, 2132, 2133, 2135, 2136, 2137, 2138, 2140, 2141, 2142, 2143, 2144, 2146, 2148, 2149, 2150, 2151, 2152, 2153, 2154, 2156, 2158, 2159, 2160, 2161, 2162, 2163, 2165, 2167, 2169, 2170, 2171, 2172, 2173, 2174, 2175, 2176, 2177, 2178, 2179, 2181, 2183, 2184, 2186, 2187, 2188, 2190, 2192, 2194, 2195, 2196, 2197, 2198, 2199, 2200, 2201, 2202, 2203, 2205, 2207, 2209, 2211, 2213, 2215, 2217, 2219, 2221, 2223, 2225, 2226, 2228, 2229, 2231, 2233, 2235, 2236, 2237, 2238, 2239, 2240, 2241, 2243, 2244, 2246, 2248, 2250, 2251, 2253, 2255, 2257, 2259, 2261, 2262, 2264, 2265, 2267, 2269, 2270, 2272, 2273, 2275, 2276, 2278, 2279, 2281, 2282, 2284, 2285, 2286, 2288, 2289, 2291, 2293, 2295, 2296, 2297, 2298, 2299, 2300, 2302, 2304, 2306, 2307, 2309, 2310, 2312, 2313, 2314, 2315, 2316, 2317, 2318, 2320, 2321, 2323, 2325, 2327, 2329, 2330, 2332, 2334, 2335, 2337, 2339, 2341, 2343, 2345, 2346, 2347, 2348, 2350, 2352, 2354, 2356, 2357, 2358, 2360, 2361, 2362, 2364, 2366, 2367, 2369, 2371, 2373, 2375, 2376, 2378, 2380, 2381, 2383, 2384, 2386, 2388, 2390, 2391, 2392, 2394, 2396, 2397, 2399, 2401, 2402, 2403, 2405, 2407, 2408, 2409, 2411, 2413, 2415, 2417, 2418, 2419, 2421, 2422, 2423, 2424, 2425, 2427, 2428, 2429, 2430, 2432, 2434, 2435, 2437, 2439, 2440, 2441, 2442, 2443, 2445, 2446, 2448, 2449, 2451, 2452, 2454, 2456, 2457, 2458, 2459, 2460, 2461, 2462, 2463, 2464, 2466, 2468, 2469, 2470, 2471, 2472, 2474, 2475, 2476, 2477, 2478, 2480, 2482, 2483, 2484, 2485, 2486, 2487, 2488, 2489, 2490, 2491, 2493, 2494, 2495, 2496, 2497, 2498, 2499, 2500, 2502, 2503, 2505, 2506, 2507, 2509, 2510, 2511, 2512, 2513, 2514, 2516, 2517, 2518, 2519, 2521, 2522, 2523, 2525, 2527, 2529, 2530, 2531, 2533, 2535, 2537, 2539, 2540, 2541, 2542, 2544, 2546, 2547, 2548, 2550, 2551, 2552, 2554, 2556, 2557, 2558, 2559, 2561, 2562, 2564, 2565, 2567, 2569, 2571, 2572, 2573, 2574, 2575, 2576, 2578, 2580, 2581, 2583, 2584, 2585, 2587, 2588, 2589, 2590, 2592, 2593, 2594, 2595, 2597, 2599, 2601, 2603, 2604, 2606, 2608, 2609, 2611, 2612, 2614, 2615, 2616, 2617, 2619, 2621, 2623, 2624, 2626, 2627, 2628, 2629, 2631, 2632, 2634, 2636, 2638, 2640, 2641, 2642, 2644, 2646, 2647, 2648, 2649, 2650, 2652, 2654, 2656, 2658, 2659, 2661, 2663, 2665, 2667, 2668, 2670, 2672, 2674, 2675, 2677, 2679, 2681, 2683, 2685, 2687, 2689, 2691, 2693, 2695, 2697, 2699, 2701, 2703, 2704, 2706, 2708, 2710, 2711, 2712, 2714, 2716, 2717, 2719, 2721, 2722, 2724, 2726, 2728, 2730, 2732, 2734, 2735, 2736, 2738, 2739, 2740, 2741, 2743, 2745, 2746, 2748, 2750, 2751, 2753, 2754, 2756, 2758, 2760, 2762, 2764, 2766, 2768, 2770, 2771, 2772, 2773, 2775, 2777, 2778, 2780, 2781, 2782, 2784, 2786, 2788, 2790, 2791, 2792, 2794, 2796, 2797, 2799, 2801, 2802, 2803, 2804, 2806, 2807, 2809, 2810, 2811, 2813, 2815, 2816, 2818, 2819, 2821, 2822, 2823, 2825, 2826, 2828, 2830, 2832, 2833, 2835, 2836, 2838, 2840, 2842, 2844, 2845, 2846, 2847, 2849, 2850, 2851, 2852, 2853, 2855, 2856, 2857, 2858, 2859, 2861, 2862, 2863, 2864, 2866, 2867, 2868, 2869, 2870, 2872, 2873, 2875, 2876, 2877, 2878, 2880, 2882, 2883, 2885, 2886, 2888, 2890, 2891, 2892, 2893, 2894, 2896, 2898, 2900, 2902, 2903, 2904, 2906, 2907, 2908, 2909, 2910, 2911, 2915, 2917, 2918, 2919, 2920, 2921, 2922, 2923, 2924, or 2926. A plant produced from the plant cell can be used to generate a plant that has a difference in the level of biomass as compared to the corresponding level of biomass of a control plant that does not comprise the exogenous nucleic acid.

In another aspect, a method comprises growing a plant cell comprising an exogenous nucleic acid. The exogenous nucleic acid comprises a regulatory region operably linked to a nucleotide sequence having 80 percent or greater sequence identity to a nucleotide sequence, or a fragment thereof, set forth in SEQ ID NO:1, 3, 7, 9, 11, 14, 16, 19, 21, 23, 28, 31, 33, 36, 40, 42, 44, 46, 48, 50, 52, 54, 58, 61, 66, 68, 72, 76, 78, 87, 89, 114, 116, 118, 125, 127, 130, 132, 134, 136, 138, 143, 145, 151, 153, 156, 159, 161, 164, 166, 168, 170, 172, 174, 177, 179, 181, 184, 186, 188, 190, 192, 202, 204, 206, 210, 212, 216, 221, 225, 227, 230, 241, 243, 253, 280, 283, 285, 290, 292, 296, 298, 301, 303, 305, 309, 312, 314, 321, 323, 325, 327, 329, 332, 334, 336, 339, 341, 344, 348, 352, 356, 359, 361, 366, 368, 376, 381, 388, 392, 394, 397, 399, 402, 405, 407, 413, 416, 418, 455, 458, 460, 463, 465, 471, 473, 476, 478, 481, 486, 496, 498, 500, 505, 508, 513, 519, 521, 523, 525, 527, 529, 541, 544, 549, 552, 556, 558, 561, 564, 567, 569, 571, 574, 578, 588, 594, 600, 603, 606, 608, 628, 630, 632, 639, 641, 643, 645, 647, 649, 652, 661, 668, 670, 673, 675, 678, 681, 683, 686, 688, 692, 695, 699, 701, 703, 708, 711, 713, 716, 724, 729, 736, 738, 740, 742, 748, 750, 754, 756, 758, 761, 763, 765, 767, 769, 771, 774, 777, 779, 781, 783, 785, 787, 790, 792, 797, 799, 803, 806, 808, 810, 812, 814, 816, 818, 821, 826, 829, 831, 833, 835, 837, 839, 841, 845, 847, 852, 854, 859, 862, 865, 867, 869, 872, 875, 877, 879, 881, 884, 886, 888, 891, 894, 898, 900, 904, 906, 911, 913, 918, 920, 922, 924, 927, 929, 931, 933, 938, 940, 942, 944, 946, 948, 951, 954, 956, 958, 961, 963, 966, 969, 971, 973, 975, 977, 979, 982, 984, 988, 990, 992, 994, 996, 999, 1001, 1004, 1006, 1010, 1012, 1015, 1017, 1019, 1021, 1028, 1030, 1032, 1035, 1037, 1040, 1047, 1053, 1055, 1057, 1059, 1063, 1065, 1068, 1073, 1075, 1080, 1082, 1084, 1086, 1089, 1091, 1094, 1098, 1100, 1103, 1106, 1109, 1116, 1118, 1123, 1125, 1127, 1129, 1131, 1134, 1136, 1138, 1140, 1143, 1145, 1147, 1149, 1151, 1153, 1155, 1159, 1161, 1163, 1165, 1167, 1170, 1172, 1174, 1178, 1182, 1184, 1186, 1189, 1191, 1195, 1197, 1199, 1202, 1204, 1206, 1208, 1211, 1213, 1215, 1217, 1220, 1223, 1225, 1227, 1229, 1231, 1233, 1237, 1239, 1242, 1244, 1246, 1248, 1250, 1254, 1256, 1258, 1260, 1262, 1264, 1267, 1269, 1271, 1273, 1275, 1277, 1280, 1282, 1284, 1286, 1288, 1290, 1292, 1295, 1298, 1301, 1303, 1305, 1307, 1311, 1315, 1317, 1319, 1321, 1323, 1327, 1331, 1335, 1337, 1346, 1348, 1351, 1354, 1356, 1362, 1365, 1368, 1374, 1378, 1383, 1385, 1387, 1390, 1392, 1394, 1396, 1398, 1400, 1404, 1406, 1408, 1411, 1413, 1415, 1417, 1419, 1423, 1425, 1427, 1430, 1432, 1434, 1436, 1438, 1442, 1444, 1447, 1449, 1451, 1453, 1455, 1458, 1460, 1462, 1464, 1466, 1468, 1470, 1472, 1474, 1476, 1478, 1480, 1484, 1487, 1491, 1494, 1496, 1499, 1501, 1504, 1510, 1513, 1517, 1519, 1521, 1527, 1530, 1532, 1536, 1539, 1541, 1543, 1545, 1548, 1551, 1553, 1556, 1559, 1561, 1566, 1568, 1570, 1573, 1576, 1579, 1582, 1585, 1587, 1590, 1592, 1597, 1601, 1606, 1608, 1610, 1614, 1617, 1620, 1623, 1625, 1627, 1631, 1633, 1635, 1638, 1641, 1643, 1645, 1651, 1653, 1655, 1659, 1663, 1665, 1667, 1673, 1675, 1678, 1685, 1687, 1690, 1692, 1694, 1696, 1698, 1703, 1709, 1716, 1718, 1720, 1722, 1726, 1728, 1730, 1733, 1736, 1738, 1740, 1742, 1744, 1748, 1750, 1752, 1754, 1756, 1760, 1763, 1768, 1771, 1775, 1778, 1780, 1782, 1785, 1787, 1789, 1791, 1793, 1796, 1800, 1802, 1814, 1818, 1820, 1824, 1830, 1832, 1836, 1838, 1840, 1843, 1846, 1848, 1851, 1860, 1862, 1864, 1868, 1870, 1873, 1875, 1878, 1880, 1884, 1886, 1888, 1890, 1892, 1896, 1899, 1901, 1905, 1907, 1909, 1911, 1914, 1916, 1921, 1923, 1925, 1927, 1930, 1932, 1934, 1937, 1939, 1941, 1943, 1945, 1947, 1950, 1952, 1954, 1956, 1958, 1960, 1962, 1964, 1966, 1968, 1970, 1977, 1981, 1984, 1988, 1991, 1993, 1995, 1998, 2001, 2004, 2006, 2012, 2014, 2020, 2025, 2027, 2029, 2033, 2035, 2039, 2045, 2050, 2052, 2054, 2057, 2059, 2061, 2063, 2065, 2068, 2071, 2080, 2082, 2084, 2086, 2089, 2094, 2096, 2099, 2101, 2103, 2107, 2109, 2111, 2114, 2116, 2123, 2125, 2127, 2131, 2134, 2139, 2145, 2147, 2155, 2157, 2164, 2166, 2168, 2180, 2182, 2185, 2189, 2191, 2193, 2204, 2206, 2208, 2210, 2212, 2214, 2216, 2218, 2220, 2222, 2224, 2227, 2230, 2232, 2234, 2242, 2245, 2247, 2249, 2252, 2254, 2256, 2258, 2260, 2263, 2266, 2268, 2271, 2274, 2277, 2280, 2283, 2287, 2290, 2292, 2294, 2301, 2303, 2305, 2308, 2311, 2319, 2322, 2324, 2326, 2328, 2331, 2333, 2336, 2338, 2340, 2342, 2344, 2349, 2351, 2353, 2355, 2359, 2363, 2365, 2368, 2370, 2372, 2374, 2377, 2379, 2382, 2385, 2387, 2389, 2393, 2395, 2398, 2400, 2404, 2406, 2410, 2412, 2414, 2416, 2420, 2426, 2431, 2433, 2436, 2438, 2444, 2447, 2450, 2453, 2455, 2465, 2467, 2473, 2479, 2481, 2492, 2501, 2504, 2508, 2515, 2520, 2524, 2526, 2528, 2532, 2534, 2536, 2538, 2543, 2545, 2549, 2553, 2555, 2560, 2563, 2566, 2568, 2570, 2577, 2579, 2582, 2586, 2591, 2596, 2598, 2600, 2602, 2605, 2607, 2610, 2613, 2618, 2620, 2622, 2625, 2630, 2633, 2635, 2637, 2639, 2643, 2645, 2651, 2653, 2655, 2657, 2660, 2662, 2664, 2666, 2669, 2671, 2673, 2676, 2678, 2680, 2682, 2684, 2686, 2688, 2690, 2692, 2694, 2696, 2698, 2700, 2702, 2705, 2707, 2709, 2713, 2715, 2718, 2720, 2723, 2725, 2727, 2729, 2731, 2733, 2737, 2742, 2744, 2747, 2749, 2752, 2755, 2757, 2759, 2761, 2763, 2765, 2767, 2769, 2774, 2776, 2779, 2783, 2785, 2787, 2789, 2793, 2795, 2798, 2800, 2805, 2808, 2812, 2814, 2817, 2820, 2824, 2827, 2829, 2831, 2834, 2837, 2839, 2841, 2843, 2848, 2854, 2860, 2865, 2871, 2874, 2879, 2881, 2884, 2887, 2889, 2895, 2897, 2899, 2901, 2905, 2912, 2914, 2916, or 2925. A plant produced from the plant cell has a difference in the level of biomass as compared to the corresponding level of biomass of a control plant that does not comprise the exogenous nucleic acid.

Methods of modulating the level of biomass in a plant are provided herein. In one aspect, a method comprises introducing into a plant cell an exogenous nucleic acid that comprises a regulatory region operably linked to a nucleotide sequence encoding a polypeptide. The HMM bit score of the amino acid sequence of the polypeptide is greater than about 915, 1149, 618, 324, 752, 866, 703, 862, 883, 393, 232, 815, 1251, 1066, 1398, 370, 328, 133, 265, 843, 93, 175, 2105, 1213, 1174, 1138, 1126, 1812, 824, 1240, 1078, 873, 1102, 754, 539, 657, 1925, 1104, 1347, 1096, 851, 761, 815, 1121, 681, 677, 146, 286, 493, or 316, using an HMM generated from the amino acid sequences depicted in one of FIGS. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, respectively. A plant produced from the plant cell has a difference in the level of biomass as compared to the corresponding level of biomass of a control plant that does not comprise the exogenous nucleic acid.

In certain embodiments, the HMM score of the amino acid sequence of the polypeptide is greater than about 915, using an HMM generated from the amino acid sequences depicted in FIG. 1, wherein the polypeptide comprises a Myb-like DNA-binding domain, having at least 60 percent or greater (e.g., 65, 70, 75, 80, 85, 90, 95, 99, or 100%) sequence identity to residues 14 to 61 or 67 to 112 of SEQ ID NO: 2880, or polypeptides comprising Myb-like DNA-binding domains identified in the sequence listing.

In certain embodiments, the HMM score of the amino acid sequence of the polypeptide is greater than about 618, using an HMM generated from the amino acid sequences depicted in FIG. 3, wherein the polypeptide comprises a K-box domain having at least 60 percent or greater (e.g., 65, 70, 75, 80, 85, 90, 95, 99, or 100%) sequence identity to residues 75 to 176 of SEQ ID NO:403 and a SRF-type transcription factor domain (DNA-binding and dimerisation domain) having at least 60 percent or greater (e.g., 65, 70, 75, 80, 85, 90, 95, 99, or 100%) sequence identity to residues 9 to 59 of SEQ ID NO:403, or polypeptides comprising Domain K-box domains and SRF-type transcription factors identified in the sequence listing.

In certain embodiments, the HMM score of the amino acid sequence of the polypeptide is greater than about 324, using an HMM generated from the amino acid sequences depicted in FIG. 4, wherein the polypeptide comprises a Basic region leucine zipper domain or a bZIP transcription factor domain having at least 60 percent or greater (e.g., 65, 70, 75, 80, 85, 90, 95, 99, or 100%) sequence identity to residues 68 to 122 of SEQ ID NO:669, or Basic region leucine zipper or bZIP transcription factor domains identified in the sequence listing.

In certain embodiments, the HMM score of the amino acid sequence of the polypeptide is greater than about 752, using an HMM generated from the amino acid sequences depicted in FIG. 5, wherein the polypeptide comprises a KNOX2 domain, a KNOX1 domain, and an ELK domain having at least 60 percent or greater (e.g., 65, 70, 75, 80, 85, 90, 95, 99, or 100%) sequence identity to residues 213 to 268, 157 to 201, and 322 to 343, respectively, of SEQ ID NO: 1797, or KNOX2, KNOX1, and ELK domains identified in the sequence listing.

In certain embodiments, the HMM score of the amino acid sequence of the polypeptide is greater than about 866, using an HMM generated from the amino acid sequences depicted in FIG. 6, wherein the polypeptide comprises a O-methyltransferase and a dimerisation domain having at least 60 percent or greater (e.g., 65, 70, 75, 80, 85, 90, 95, 99, or 100%) sequence identity to residues 31 to 82 and 91 to 255 of SEQ ID NO:717, or O-methyltransferase and dimerisation domains identified in the sequence listing.

In certain embodiments, the HMM score of the amino acid sequence of the polypeptide is greater than about 703, using an HMM generated from the amino acid sequences depicted in FIG. 7, wherein the polypeptide comprises an AP2 domain having at least 60 percent or greater (e.g., 65, 70, 75, 80, 85, 90, 95, 99, or 100%) sequence identity to residues 209 to 260 of SEQ ID NO:693, or AP2 domains identified in the sequence listing.

In certain embodiments, the HMM score of the amino acid sequence of the polypeptide is greater than about 862, using an HMM generated from the amino acid sequences depicted in FIG. 8, wherein the polypeptide comprises a Fasciclin domain having at least 60 percent or greater (e.g., 65, 70, 75, 80, 85, 90, 95, 99, or 100%) sequence identity to residues 202 to 334 of SEQ ID NO:1517, or Fasciclin domains identified in the sequence listing.

In certain embodiments, the HMM score of the amino acid sequence of the polypeptide is greater than about 883, using an HMM generated from the amino acid sequences depicted in FIG. 9, wherein the polypeptide comprises a NAD dependent epimerase/dehydratase family domain having at least 60 percent or greater (e.g., 65, 70, 75, 80, 85, 90, 95, 99, or 100%) sequence identity to residues 21 to 253 of SEQ ID NO: 1624, or NAD dependent epimerase/dehydratase family domains identified in the sequence listing.

In certain embodiments, the HMM score of the amino acid sequence of the polypeptide is greater than about 393, using an HMM generated from the amino acid sequences depicted in FIG. 10, wherein the polypeptide comprises a X8 domain having at least 60 percent or greater (e.g., 65, 70, 75, 80, 85, 90, 95, 99, or 100%) sequence identity to residues 21 to 106 of SEQ ID NO:1887, or X8 domains identified in the sequence listing.

In certain embodiments, the HMM score of the amino acid sequence of the polypeptide is greater than about 815, using an HMM generated from the amino acid sequences depicted in FIG. 12, wherein the polypeptide comprises a peroxidase domain having at least 60 percent or greater (e.g., 65, 70, 75, 80, 85, 90, 95, 99, or 100%) sequence identity to residues 88 to 453 of SEQ ID NO:1318, or peroxidase domains identified in the sequence listing.

In certain embodiments, the HMM score of the amino acid sequence of the polypeptide is greater than about 1251, using an HMM generated from the amino acid sequences depicted in FIG. 13, wherein the polypeptide comprises a DUF563 domain having at least 60 percent or greater (e.g., 65, 70, 75, 80, 85, 90, 95, 99, or 100%) sequence identity to residues 109 to 357 of SEQ ID NO:459, or DUF563 domains identified in the sequence listing.

In certain embodiments, the HMM score of the amino acid sequence of the polypeptide is greater than about 1066, using an HMM generated from the amino acid sequences depicted in FIG. 14, wherein the polypeptide comprises a UDP-glucoronosyl and UDP-glucosyl transferase domain having at least 60 percent or greater (e.g., 65, 70, 75, 80, 85, 90, 95, 99, or 100%) sequence identity to residues 4 to 457 of SEQ ID NO:335, or UDP-glucoronosyl and UDP-glucosyl transferase domains identified in the sequence listing.

In certain embodiments, the HMM score of the amino acid sequence of the polypeptide is greater than about 1398, using an HMM generated from the amino acid sequences depicted in FIG. 15, wherein the polypeptide comprises a DUF563 domain having at least 60 percent or greater (e.g., 65, 70, 75, 80, 85, 90, 95, 99, or 100%) sequence identity to residues 98 to 342 of SEQ ID NO: 846, or DUF563 domains identified in the sequence listing.

In certain embodiments, the HMM score of the amino acid sequence of the polypeptide is greater than about 370, using an HMM generated from the amino acid sequences depicted in FIG. 16, wherein the polypeptide comprises an aminotransferase class I and II domain having at least 60 percent or greater (e.g., 65, 70, 75, 80, 85, 90, 95, 99, or 100%) sequence identity to residues 88 to 453 of SEQ ID NO: 645, or Aminotransferase class I and II domains identified in the sequence listing.

In certain embodiments, the HMM score of the amino acid sequence of the polypeptide is greater than about 328, using an HMM generated from the amino acid sequences depicted in FIG. 17, wherein the polypeptide comprises a Dirigent-like protein domain having at least 60 percent or greater (e.g., 65, 70, 75, 80, 85, 90, 95, 99, or 100%) sequence identity to residues 1 to 146 of SEQ ID NO: 2525, or Dirigent-like protein domains identified in the sequence listing.

In certain embodiments, the HMM score of the amino acid sequence of the polypeptide is greater than about 133, using an HMM generated from the amino acid sequences depicted in FIG. 18, wherein the polypeptide comprises a DUF1070 domain having at least 60 percent or greater (e.g., 65, 70, 75, 80, 85, 90, 95, 99, or 100%) sequence identity to residues 8 to 61 of SEQ ID NO: 1272, or DUF1070 domains identified in the sequence listing.

In certain embodiments, the HMM score of the amino acid sequence of the polypeptide is greater than about 843, using an HMM generated from the amino acid sequences depicted in FIG. 20, wherein the polypeptide comprises a Pectinesterase domain having at least 60 percent or greater (e.g., 65, 70, 75, 80, 85, 90, 95, 99, or 100%) sequence identity to residues 88 to 453 of SEQ ID NO: 1660, or Pectinesterase domains identified in the sequence listing.

In certain embodiments, the HMM score of the amino acid sequence of the polypeptide is greater than about 175, using an HMM generated from the amino acid sequences depicted in FIG. 22, wherein the polypeptide comprises a Myb-like DNA-binding domain having at least 60 percent or greater (e.g., 65, 70, 75, 80, 85, 90, 95, 99, or 100%) sequence identity to residues 10 to 59 of SEQ ID NO: 1137, or Myb-like DNA-binding domains identified in the sequence listing.

In certain embodiments, the HMM score of the amino acid sequence of the polypeptide is greater than about 2105, using an HMM generated from the amino acid sequences depicted in FIG. 23, wherein the polypeptide comprises a Glycosyl hydrolase family 35 domain and a Galactose binding lectin domain having at least 60 percent or greater (e.g., 65, 70, 75, 80, 85, 90, 95, 99, or 100%) sequence identity to residues 70 to 376 and 830 to 906 of SEQ ID NO: 2466, or Glycosyl hydrolase family 35 and Galactose binding lectin domains identified in the sequence listing.

In certain embodiments, the HMM score of the amino acid sequence of the polypeptide is greater than about 1213, using an HMM generated from the amino acid sequences depicted in FIG. 24, wherein the polypeptide comprises a DUF563 domain having at least 60 percent or greater (e.g., 65, 70, 75, 80, 85, 90, 95, 99, or 100%) sequence identity to residues 252 to 501 of SEQ ID NO:1106, or DUF563 domains identified in the sequence listing.

In certain embodiments, the HMM score of the amino acid sequence of the polypeptide is greater than about 1174, using an HMM generated from the amino acid sequences depicted in FIG. 25, wherein the polypeptide comprises a UDP-glucoronosyl and UDP-glucosyl transferases domain having at least 60 percent or greater (e.g., 65, 70, 75, 80, 85, 90, 95, 99, or 100%) sequence identity to residues 24 to 465 of SEQ ID NO: 1946, or UDP-glucoronosyl and UDP-glucosyl transferases domains identified in the sequence listing.

In certain embodiments, the HMM score of the amino acid sequence of the polypeptide is greater than about 1138, using an HMM generated from the amino acid sequences depicted in FIG. 26, wherein the polypeptide comprises a NAD dependent epimerase/dehydratase family domain having at least 60 percent or greater (e.g., 65, 70, 75, 80, 85, 90, 95, 99, or 100%) sequence identity to residues 20 to 290 of SEQ ID NO:1054, or Epimerase domains identified in the sequence listing.

In certain embodiments, the HMM score of the amino acid sequence of the polypeptide is greater than about 1126, using an HMM generated from the amino acid sequences depicted in FIG. 27, wherein the polypeptide comprises an Exostosin domain having at least 60 percent or greater (e.g., 65, 70, 75, 80, 85, 90, 95, 99, or 100%) sequence identity to residues 54 to 358 of SEQ ID NO:115, or Exostosin domains identified in the sequence listing.

In certain embodiments, the HMM score of the amino acid sequence of the polypeptide is greater than about 1812, using an HMM generated from the amino acid sequences depicted in FIG. 28, wherein the polypeptide comprises a NAD dependent epimerase/dehydratase family domain and a RmlD substrate binding domain having at least 60 percent or greater (e.g., 65, 70, 75, 80, 85, 90, 95, 99, or 100%) sequence identity to residues 10 to 253 and 381 to 659 of SEQ ID NO:2708, or a NAD dependent epimerase/dehydratase family and RmlD substrate binding domains identified in the sequence listing.

In certain embodiments, the HMM score of the amino acid sequence of the polypeptide is greater than about 824, using an HMM generated from the amino acid sequences depicted in FIG. 29, wherein the polypeptide comprises a Glycosyltransferase family 43 domain having at least 60 percent or greater (e.g., 65, 70, 75, 80, 85, 90, 95, 99, or 100%) sequence identity to residues 138 to 364 of SEQ ID NO:1481, or Glycosyltransferase family 43 domains identified in the sequence listing.

In certain embodiments, the HMM score of the amino acid sequence of the polypeptide is greater than about 1240, using an HMM generated from the amino acid sequences depicted in FIG. 30, wherein the polypeptide comprises a Glycosyl hydrolase family 17 domain and a X8 domain having at least 60 percent or greater (e.g., 65, 70, 75, 80, 85, 90, 95, 99, or 100%) sequence identity to residues 30 to 348 and 368 to 452 of SEQ ID NO:2748, or Glycosyl hydrolase family 17 and X8 domains identified in the sequence listing.

In certain embodiments, the HMM score of the amino acid sequence of the polypeptide is greater than about 1078, using an HMM generated from the amino acid sequences depicted in FIG. 31, wherein the polypeptide comprises a Transferase family domain having at least 60 percent or greater (e.g., 65, 70, 75, 80, 85, 90, 95, 99, or 100%) sequence identity to residues 5 to 439 of SEQ ID NO:947, or Transferase family domains identified in the sequence listing.

In certain embodiments, the HMM score of the amino acid sequence of the polypeptide is greater than about 873, using an HMM generated from the amino acid sequences depicted in FIG. 32, wherein the polypeptide comprises an alpha/beta hydrolase fold domain having at least 60 percent or greater (e.g., 65, 70, 75, 80, 85, 90, 95, 99, or 100%) sequence identity to residues 88 to 453 of SEQ ID NO:1727, or alpha/beta hydrolase fold domains identified in the sequence listing.

In certain embodiments, the HMM score of the amino acid sequence of the polypeptide is greater than about 1102, using an HMM generated from the amino acid sequences depicted in FIG. 33, wherein the polypeptide comprises a UDP-glycoronosyl and UDP-glucosyl transferases domain having at least 60 percent or greater (e.g., 65, 70, 75, 80, 85, 90, 95, 99, or 100%) sequence identity to residues 24 to 247 of SEQ ID NO: 2146, or UDP-glycoronosyl and UDP-glucosyl transferases domains identified in the sequence listing.

In certain embodiments, the HMM score of the amino acid sequence of the polypeptide is greater than about 754, using an HMM generated from the amino acid sequences depicted in FIG. 34, wherein the polypeptide comprises a Myb-like DNA-binding domain having at least 60 percent or greater (e.g., 65, 70, 75, 80, 85, 90, 95, 99, or 100%) sequence identity to residues 223 to 274 of SEQ ID NO:2619, or Myb-like DNA binding domains identified in the sequence listing.

In certain embodiments, the HMM score of the amino acid sequence of the polypeptide is greater than about 539, using an HMM generated from the amino acid sequences depicted in FIG. 35, wherein the polypeptide comprises a Chitinase class I domain having at least 60 percent or greater (e.g., 65, 70, 75, 80, 85, 90, 95, 99, or 100%) sequence identity to residues 29 to 246 of SEQ ID NO:211, or Chitinase class I domains identified in the sequence listing.

In certain embodiments, the HMM score of the amino acid sequence of the polypeptide is greater than about 657, using an HMM generated from the amino acid sequences depicted in FIG. 36, wherein the polypeptide comprises a Pollen allergen domain and a rare lipoprotein A (RlpA)-like double psi beta barrel domain having at least 60 percent or greater (e.g., 65, 70, 75, 80, 85, 90, 95, 99, or 100%) sequence identity to residues 164 to 241 and 67 to 153 of SEQ ID NO: 629, or Pollen allergen and rare lipoprotein A (RlpA)-like double psi beta barrel domains identified in the sequence listing.

In certain embodiments, the HMM score of the amino acid sequence of the polypeptide is greater than about 1925, using an HMM generated from the amino acid sequences depicted in FIG. 37, wherein the polypeptide comprises a Raffinose synthase or seed inhibition (Sip1) protein domain having at least 60 percent or greater (e.g., 65, 70, 75, 80, 85, 90, 95, 99, or 100%) sequence identity to residues 7 to 752 of SEQ ID NO:2544, or Raffinose synthase or seed inhibition (Sip1) protein domains identified in the sequence listing.

In certain embodiments, the HMM score of the amino acid sequence of the polypeptide is greater than about 1104, using an HMM generated from the amino acid sequences depicted in FIG. 38, wherein the polypeptide comprises a Glycosyl hydrolase family 28 domain having at least 60 percent or greater (e.g., 65, 70, 75, 80, 85, 90, 95, 99, or 100%) sequence identity to residues 59 to 417 of SEQ ID NO:2599, or Glycosyl hydrolase family 28 domains identified in the sequence listing.

In certain embodiments, the HMM score of the amino acid sequence of the polypeptide is greater than about 1347, using an HMM generated from the amino acid sequences depicted in FIG. 39, wherein the polypeptide comprises a 4-alpha-glucanotransferase domain having at least 60 percent or greater (e.g., 65, 70, 75, 80, 85, 90, 95, 99, or 100%) sequence identity to residues 90 to 579 of SEQ ID NO: 2437, or 4-alpha-glucanotransferase domains identified in the sequence listing.

In certain embodiments, the HMM score of the amino acid sequence of the polypeptide is greater than about 1096, using an HMM generated from the amino acid sequences depicted in FIG. 40, wherein the polypeptide comprises a NAD dependent epimerase/dehydratase family domain having at least 60 percent or greater (e.g., 65, 70, 75, 80, 85, 90, 95, 99, or 100%) sequence identity to residues 79 to 342 of SEQ ID NO: 2090, or NAD dependent epimerase/dehydratase family domains identified in the sequence listing.

In certain embodiments, the HMM score of the amino acid sequence of the polypeptide is greater than about 851, using an HMM generated from the amino acid sequences depicted in FIG. 41, wherein the polypeptide comprises a NAD dependent epimerase/dehydratase family domain having at least 60 percent or greater (e.g., 65, 70, 75, 80, 85, 90, 95, 99, or 100%) sequence identity to residues 14 to 258 of SEQ ID NO: 2323, or NAD dependent epimerase/dehydratase family domains identified in the sequence listing.

In certain embodiments, the HMM score of the amino acid sequence of the polypeptide is greater than about 761, using an HMM generated from the amino acid sequences depicted in FIG. 42, wherein the polypeptide comprises a No apical meristem (NAM) protein domain having at least 60 percent or greater (e.g., 65, 70, 75, 80, 85, 90, 95, 99, or 100%) sequence identity to residues 12 to 154 of SEQ ID NO:2, or No apical meristem (NAM) protein domains identified in the sequence listing.

In certain embodiments, the HMM score of the amino acid sequence of the polypeptide is greater than about 815, using an HMM generated from the amino acid sequences depicted in FIG. 43, wherein the polypeptide comprises a NAD dependent epimerase/dehydratase family domain having at least 60 percent or greater (e.g., 65, 70, 75, 80, 85, 90, 95, 99, or 100%) sequence identity to residues 13 to 258 of SEQ ID NO: 2028, or NAD dependent epimerase/dehydratase family domains identified in the sequence listing.

In certain embodiments, the HMM score of the amino acid sequence of the polypeptide is greater than about 1121, using an HMM generated from the amino acid sequences depicted in FIG. 44, wherein the polypeptide comprises a Transferase family domain having at least 60 percent or greater (e.g., 65, 70, 75, 80, 85, 90, 95, 99, or 100%) sequence identity to residues 4 to 436 of SEQ ID NO:1569, or Transferase family domains identified in the sequence listing.

In certain embodiments, the HMM score of the amino acid sequence of the polypeptide is greater than about 681, using an HMM generated from the amino acid sequences depicted in FIG. 45, wherein the polypeptide comprises a Glycosyl hydrolase family 18 domain having at least 60 percent or greater (e.g., 65, 70, 75, 80, 85, 90, 95, 99, or 100%) sequence identity to residues 26 to 290 of SEQ ID NO:501, or Glycosyl hydrolase family 18 domains identified in the sequence listing.

In certain embodiments, the HMM score of the amino acid sequence of the polypeptide is greater than about 677, using an HMM generated from the amino acid sequences depicted in FIG. 46, wherein the polypeptide comprises a Glycosyl hydrolase family 16 domain and a Xyloglucan endo-transglycosylase (XET) domain having at least 60 percent or greater (e.g., 65, 70, 75, 80, 85, 90, 95, 99, or 100%) sequence identity to residues 23 to 204 and 228 to 280 of SEQ ID NO:2190, or a Glycosyl hydrolase family 16 and Xyloglucan endo-transglycosylase (XET) domains identified in the sequence listing.

In certain embodiments, the HMM score of the amino acid sequence of the polypeptide is greater than about 146, using an HMM generated from the amino acid sequences depicted in FIG. 47, wherein the polypeptide comprises a Potato inhibitor I family domain having at least 60 percent or greater (e.g., 65, 70, 75, 80, 85, 90, 95, 99, or 100%) sequence identity to residues 17 to 76 of SEQ ID NO: 749, or potato inhibitor I family domains identified in the sequence listing.

In certain embodiments, the HMM score of the amino acid sequence of the polypeptide is greater than about 286, using an HMM generated from the amino acid sequences depicted in FIG. 48, wherein the polypeptide comprises a Plant Thionin domain having at least 60 percent or greater (e.g., 65, 70, 75, 80, 85, 90, 95, 99, or 100%) sequence identity to residues 25 to 67 of SEQ ID NO:79, or Plant Thionin domains identified in the sequence listing.

In certain embodiments, the HMM score of the amino acid sequence of the polypeptide is greater than about 493, using an HMM generated from the amino acid sequences depicted in FIG. 49, wherein the polypeptide comprises a No apical meristem (NAM) protein domain having at least 60 percent or greater (e.g., 65, 70, 75, 80, 85, 90, 95, 99, or 100%) sequence identity to residues 7 to 125 of SEQ ID NO:284, or No apical meristem (NAM) protein domains identified in the sequence listing.

In certain embodiments, the HMM score of the amino acid sequence of the polypeptide is greater than about 316, using an HMM generated from the amino acid sequences depicted in FIG. 50, wherein the polypeptide comprises a HMA domain having at least 60 percent or greater (e.g., 65, 70, 75, 80, 85, 90, 95, 99, or 100%) sequence identity to residues 6 to 73 of SEQ ID NO:2915, or HMA domains identified in the sequence listing.

In another aspect, a method comprises introducing into a plant cell an exogenous nucleic acid that comprises a regulatory region operably linked to a nucleotide sequence encoding a polypeptide having 80 percent or greater sequence identity to an amino acid sequence set forth in SEQ ID NO:2, 4, 5, 6, 8, 10, 12, 13, 15, 17, 18, 20, 22, 24, 25, 26, 27, 29, 30, 32, 34, 35, 37, 38, 39, 41, 43, 45, 47, 49, 51, 53, 55, 56, 57, 59, 60, 62, 63, 64, 65, 67, 69, 70, 71, 73, 74, 75, 77, 79, 80, 81, 82, 83, 84, 85, 86, 88, 90, 91, 92, 93, 94, 95, 96, 97, 98, 99, 100, 101, 102, 103, 104, 105, 106, 107, 108, 109, 110, 111, 112, 113, 115, 117, 119, 120, 121, 122, 123, 124, 126, 128, 129, 131, 133, 135, 137, 139, 140, 141, 142, 144, 146, 147, 148, 149, 150, 152, 154, 155, 157, 158, 160, 162, 163, 165, 167, 169, 171, 173, 175, 176, 178, 180, 182, 183, 185, 187, 189, 191, 193, 194, 195, 196, 197, 198, 199, 200, 201, 203, 205, 207, 208, 209, 211, 213, 214, 215, 217, 218, 219, 220, 222, 223, 224, 226, 228, 229, 231, 232, 233, 234, 235, 236, 237, 238, 239, 240, 242, 244, 245, 246, 247, 248, 249, 250, 251, 252, 254, 255, 256, 257, 258, 259, 260, 261, 262, 263, 264, 265, 266, 267, 268, 269, 270, 271, 272, 273, 274, 275, 276, 277, 278, 279, 281, 282, 284, 286, 287, 288, 289, 291, 293, 294, 295, 297, 299, 300, 302, 304, 306, 307, 308, 310, 311, 313, 315, 316, 317, 318, 319, 320, 322, 324, 326, 328, 330, 331, 333, 335, 337, 338, 340, 342, 343, 345, 346, 347, 349, 350, 351, 353, 354, 355, 357, 358, 360, 362, 363, 364, 365, 367, 369, 370, 371, 372, 373, 374, 375, 377, 378, 379, 380, 382, 383, 384, 385, 386, 387, 389, 390, 391, 393, 395, 396, 398, 400, 401, 403, 404, 406, 408, 409, 410, 411, 412, 414, 415, 417, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 456, 457, 459, 461, 462, 464, 466, 467, 468, 469, 470, 472, 474, 475, 477, 479, 480, 482, 483, 484, 485, 487, 488, 489, 490, 491, 492, 493, 494, 495, 497, 499, 501, 502, 503, 504, 506, 507, 509, 510, 511, 512, 514, 515, 516, 517, 518, 520, 522, 524, 526, 528, 530, 531, 532, 533, 534, 535, 536, 537, 538, 539, 540, 542, 543, 545, 546, 547, 548, 550, 551, 553, 554, 555, 557, 559, 560, 562, 563, 565, 566, 568, 570, 572, 573, 575, 576, 577, 579, 580, 581, 582, 583, 584, 585, 586, 587, 589, 590, 591, 592, 593, 595, 596, 597, 598, 599, 601, 602, 604, 605, 607, 609, 610, 611, 612, 613, 614, 615, 616, 617, 618, 619, 620, 621, 622, 623, 624, 625, 626, 627, 629, 631, 633, 634, 635, 36, 637, 638, 640, 642, 644, 646, 648, 650, 651, 653, 654, 655, 656, 657, 658, 659, 660, 662, 663, 664, 665, 666, 667, 669, 671, 672, 674, 676, 677, 679, 680, 682, 684, 685, 687, 689, 690, 691, 693, 694, 696, 697, 698, 00, 702, 704, 705, 706, 707, 709, 710, 712, 714, 715, 717, 718, 719, 720, 721, 722, 723, 725, 726, 727, 728, 730, 731, 732, 733, 734, 735, 737, 739, 741, 743, 744, 745, 746, 747, 749, 751, 752, 753, 755, 757, 759, 760, 762, 764, 766, 768, 770, 772, 773, 775, 776, 778, 780, 782, 784, 786, 788, 789, 791, 793, 794, 795, 796, 798, 800, 801, 802, 804, 805, 807, 809, 811, 813, 815, 817, 819, 820, 822, 823, 824, 825, 827, 828, 830, 832, 834, 836, 838, 840, 842, 843, 844, 846, 848, 849, 850, 851, 853, 855, 856, 857, 858, 860, 861, 863, 864, 866, 868, 870, 871, 873, 874, 876, 878, 880, 882, 883, 885, 887, 889, 890, 892, 893, 895, 896, 897, 899, 901, 902, 903, 905, 907, 908, 909, 910, 912, 914, 915, 916, 917, 919, 921, 923, 925, 926, 928, 930, 932, 934, 935, 936, 937, 939, 941, 943, 945, 947, 949, 950, 952, 953, 955, 957, 959, 960, 962, 964, 965, 967, 968, 970, 972, 974, 976, 978, 980, 981, 983, 985, 86, 87, 989, 991, 993, 995, 997, 998, 1000, 1002, 1003, 1005, 1007, 1008, 1009, 1011, 1013, 1014, 1016, 1018, 1020, 1022, 1023, 1024, 1025, 1026, 1027, 1029, 1031, 1033, 1034, 1036, 1038, 1039, 1041, 1042, 1043, 1044, 1045, 1046, 1048, 1049, 1050, 1051, 1052, 1054, 1056, 1058, 1060, 1061, 1062, 1064, 1066, 1067, 1069, 1070, 1071, 1072, 1074, 1076, 1077, 1078, 1079, 1081, 1083, 1085, 1087, 1088, 1090, 1092, 1093, 1095, 1096, 1097, 1099, 1101, 1102, 1104, 1105, 1107, 1108, 1110, 1111, 1112, 1113, 1114, 1115, 1117, 1119, 1120, 1121, 1122, 1124, 1126, 1128, 1130, 1132, 1133, 1135, 1137, 1139, 1141, 1142, 1144, 1146, 1148, 1150, 1152, 1154, 1156, 1157, 1158, 1160, 1162, 1164, 1166, 1168, 1169, 1171, 1173, 1175, 1176, 1177, 1179, 1180, 1181, 1183, 1185, 1187, 1188, 1190, 1192, 1193, 1194, 1196, 1198, 1200, 1201, 1203, 1205, 1207, 1209, 1210, 1212, 1214, 1216, 1218, 1219, 1221, 1222, 1224, 1226, 1228, 1230, 1232, 1234, 1235, 1236, 1238, 1240, 1241, 1243, 1245, 1247, 1249, 1251, 1252, 1253, 1255, 1257, 1259, 1261, 1263, 1265, 1266, 1268, 1270, 1272, 1274, 1276, 1278, 1279, 1281, 1283, 1285, 1287, 1289, 1291, 1293, 1294, 1296, 1297, 1299, 1300, 1302, 1304, 1306, 1308, 1309, 1310, 1312, 1313, 1314, 1316, 1318, 1320, 1322, 1324, 1325, 1326, 1328, 1329, 1330, 1332, 1333, 1334, 1336, 1338, 1339, 1340, 1341, 1342, 1343, 1344, 1345, 1347, 1349, 1350, 1352, 1353, 1355, 1357, 1358, 1359, 1360, 1361, 1363, 1364, 1366, 1367, 1369, 1370, 1371, 1372, 1373, 1375, 1376, 1377, 1379, 1380, 1381, 1382, 1384, 1386, 1388, 1389, 1391, 1393, 1395, 1397, 1399, 1401, 1402, 1403, 1405, 1407, 1409, 1410, 1412, 1414, 1416, 1418, 1420, 1421, 1422, 1424, 1426, 1428, 1429, 1431, 1433, 1435, 1437, 1439, 1440, 1441, 1443, 1445, 1446, 1448, 1450, 1452, 1454, 1456, 1457, 1459, 1461, 1463, 1465, 1467, 1469, 1471, 1473, 1475, 1477, 1479, 1481, 1482, 1483, 1485, 1486, 1488, 1489, 1490, 1492, 1493, 1495, 1497, 1498, 1500, 1502, 1503, 1505, 1506, 1507, 1508, 1509, 1511, 1512, 1514, 1515, 1516, 1518, 1520, 1522, 1523, 1524, 1525, 1526, 1528, 1529, 1531, 1533, 1534, 1535, 1537, 1538, 1540, 1542, 1544, 1546, 1547, 1549, 1550, 1552, 1554, 1555, 1557, 1558, 1560, 1562, 1563, 1564, 1565, 1568, 1569, 1571, 1572, 1574, 1575, 1577, 1578, 1580, 1581, 1583, 1584, 1586, 1588, 1589, 1591, 1593, 1594, 1595, 1596, 1598, 1599, 1600, 1602, 1603, 1604, 1605, 1607, 1609, 1611, 1612, 1613, 1615, 1616, 1618, 1619, 1621, 1622, 1624, 1626, 1628, 1629, 1630, 1632, 1634, 1636, 1637, 1639, 1640, 1642, 1644, 1646, 1647, 1648, 1649, 1650, 1652, 1654, 1656, 1657, 1658, 1660, 1661, 1662, 1664, 1666, 1668, 1669, 1670, 1671, 1672, 1674, 1676, 1677, 1679, 1680, 1681, 1682, 1683, 1684, 1686, 1688, 1689, 1691, 1693, 1695, 1697, 1699, 1700, 1701, 1702, 1704, 1705, 1706, 1707, 1708, 1710, 1711, 1712, 1713, 1714, 1715, 1717, 1719, 1721, 1723, 1724, 1725, 1727, 1729, 1731, 1732, 1734, 1735, 1737, 1739, 1741, 1743, 1745, 1746, 1747, 1749, 1751, 1753, 1755, 1757, 1758, 1759, 1761, 1762, 1764, 1765, 1766, 1767, 1769, 1770, 1772, 1773, 1774, 1776, 1777, 1779, 1781, 1783, 1784, 1786, 1788, 1790, 1792, 1794, 1795, 1797, 798, 1799, 1801, 1803, 1804, 1805, 1806, 1807, 1808, 1809, 1810, 1811, 1812, 1813, 1815, 1816, 1817, 1819, 1821, 1822, 1823, 1825, 1826, 1827, 1828, 1829, 1831, 1833, 1834, 1835, 1837, 1839, 1841, 1842, 1844, 1845, 1847, 1849, 1850, 1852, 1853, 1854, 1855, 1856, 1857, 1858, 1859, 1861, 1863, 1865, 1866, 1867, 1869, 1871, 1872, 1874, 1876, 1877, 1879, 1881, 1882, 1883, 1885, 1887, 1889, 1891, 1893, 1894, 1895, 1897, 1898, 1900, 1902, 1903, 1904, 1906, 1908, 1910, 1912, 1913, 1915, 1917, 1918, 1919, 1920, 1922, 1924, 1926, 1928, 1929, 1931, 1933, 1935, 1936, 1938, 1940, 1942, 1944, 1946, 1948, 1949, 1951, 1953, 1955, 1957, 1959, 1961, 1963, 1965, 1967, 1969, 1971, 1972, 1973, 1974, 1975, 1976, 1978, 1979, 1980, 1982, 1983, 1985, 1986, 1987, 1989, 1990, 1992, 1994, 1996, 1997, 1999, 2000, 2002, 2003, 2005, 2007, 2008, 2009, 2010, 2011, 2013, 2015, 2016, 2017, 2018, 2019, 2021, 2022, 2023, 2024, 2026, 2028, 2030, 2031, 2032, 2034, 2036, 2037, 2038, 2040, 2041, 2042, 2043, 2044, 2046, 2047, 2048, 2049, 2051, 2053, 2055, 2056, 2058, 2060, 2062, 2064, 2066, 2067, 2069, 2070, 2072, 2073, 2074, 2075, 2076, 2077, 2078, 2079, 2081, 2083, 2085, 2087, 2088, 2090, 2091, 2092, 2093, 2095, 2097, 2098, 2100, 2102, 2104, 2105, 2106, 2108, 2110, 2112, 2113, 2115, 2117, 2118, 2119, 2120, 2121, 2122, 2124, 2126, 2128, 2129, 2130, 2132, 2133, 2135, 2136, 2137, 2138, 2140, 2141, 2142, 2143, 2144, 2146, 2148, 2149, 2150, 2151, 2152, 2153, 2154, 2156, 2158, 2159, 2160, 2161, 2162, 2163, 2165, 2167, 2169, 2170, 2171, 2172, 2173, 2174, 2175, 2176, 2177, 2178, 2179, 2181, 2183, 2184, 2186, 2187, 2188, 2190, 2192, 2194, 2195, 2196, 2197, 2198, 2199, 2200, 2201, 2202, 2203, 2205, 2207, 2209, 2211, 2213, 2215, 2217, 2219, 2221, 2223, 2225, 2226, 2228, 2229, 2231, 2233, 2235, 2236, 2237, 2238, 2239, 2240, 2241, 2243, 2244, 2246, 2248, 2250, 2251, 2253, 2255, 2257, 2259, 2261, 2262, 2264, 2265, 2267, 2269, 2270, 2272, 2273, 2275, 2276, 2278, 2279, 2281, 2282, 2284, 2285, 2286, 2288, 2289, 2291, 2293, 2295, 2296, 2297, 2298, 2299, 2300, 2302, 2304, 2306, 2307, 2309, 2310, 2312, 2313, 2314, 2315, 2316, 2317, 2318, 2320, 2321, 2323, 2325, 2327, 2329, 2330, 2332, 2334, 2335, 2337, 2339, 2341, 2343, 2345, 2346, 2347, 2348, 2350, 2352, 2354, 2356, 2357, 2358, 2360, 2361, 2362, 2364, 2366, 2367, 2369, 2371, 2373, 2375, 2376, 2378, 2380, 2381, 2383, 2384, 2386, 2388, 2390, 2391, 2392, 2394, 2396, 2397, 2399, 2401, 2402, 2403, 2405, 2407, 2408, 2409, 2411, 2413, 2415, 2417, 2418, 2419, 2421, 2422, 2423, 2424, 2425, 2427, 2428, 2429, 2430, 2432, 2434, 2435, 2437, 2439, 2440, 2441, 2442, 2443, 2445, 2446, 2448, 2449, 2451, 2452, 2454, 2456, 2457, 2458, 2459, 2460, 2461, 2462, 2463, 2464, 2466, 2468, 2469, 2470, 2471, 2472, 2474, 2475, 2476, 2477, 2478, 2480, 2482, 2483, 2484, 2485, 2486, 2487, 2488, 2489, 2490, 2491, 2493, 2494, 2495, 2496, 2497, 2498, 2499, 2500, 2502, 2503, 2505, 2506, 2507, 2509, 2510, 2511, 2512, 2513, 2514, 2516, 2517, 2518, 2519, 2521, 2522, 2523, 2525, 2527, 2529, 2530, 2531, 2533, 2535, 2537, 2539, 2540, 2541, 2542, 2544, 2546, 2547, 2548, 2550, 2551, 2552, 2554, 2556, 2557, 2558, 2559, 2561, 2562, 2564, 2565, 2567, 2569, 2571, 2572, 2573, 2574, 2575, 2576, 2578, 2580, 2581, 2583, 2584, 2585, 2587, 2588, 2589, 2590, 2592, 2593, 2594, 2595, 2597, 2599, 2601, 2603, 2604, 2606, 2608, 2609, 2611, 2612, 2614, 2615, 2616, 2617, 2619, 2621, 2623, 2624, 2626, 2627, 2628, 2629, 2631, 2632, 2634, 2636, 2638, 2640, 2641, 2642, 2644, 2646, 2647, 2648, 2649, 2650, 2652, 2654, 2656, 2658, 2659, 2661, 2663, 2665, 2667, 2668, 2670, 2672, 2674, 2675, 2677, 2679, 2681, 2683, 2685, 2687, 2689, 2691, 2693, 2695, 2697, 2699, 2701, 2703, 2704, 2706, 2708, 2710, 2711, 2712, 2714, 2716, 2717, 2719, 2721, 2722, 2724, 2726, 2728, 2730, 2732, 2734, 2735, 2736, 2738, 2739, 2740, 2741, 2743, 2745, 2746, 2748, 2750, 2751, 2753, 2754, 2756, 2758, 2760, 2762, 2764, 2766, 2768, 2770, 2771, 2772, 2773, 2775, 2777, 2778, 2780, 2781, 2782, 2784, 2786, 2788, 2790, 2791, 2792, 2794, 2796, 2797, 2799, 2801, 2802, 2803, 2804, 2806, 2807, 2809, 2810, 2811, 2813, 2815, 2816, 2818, 2819, 2821, 2822, 2823, 2825, 2826, 2828, 2830, 2832, 2833, 2835, 2836, 2838, 2840, 2842, 2844, 2845, 2846, 2847, 2849, 2850, 2851, 2852, 2853, 2855, 2856, 2857, 2858, 2859, 2861, 2862, 2863, 2864, 2866, 2867, 2868, 2869, 2870, 2872, 2873, 2875, 2876, 2877, 2878, 2880, 2882, 2883, 2885, 2886, 2888, 2890, 2891, 2892, 2893, 2894, 2896, 2898, 2900, 2902, 2903, 2904, 2906, 2907, 2908, 2909, 2910, 2911, 2915, 2917, 2918, 2919, 2920, 2921, 2922, 2923, 2924, or 2926. A plant produced from the plant cell has a difference in the level of biomass as compared to the corresponding level of biomass of a control plant that does not comprise the exogenous nucleic acid. The polypeptide in any of the above methods can have the amino acid sequence set forth in SEQ ID NO:2, 4, 5, 6, 8, 10, 12, 13, 15, 17, 18, 20, 22, 24, 25, 26, 27, 29, 30, 32, 34, 35, 37, 38, 39, 41, 43, 45, 47, 49, 51, 53, 55, 56, 57, 59, 60, 62, 63, 64, 65, 67, 69, 70, 71, 73, 74, 75, 77, 79, 80, 81, 82, 83, 84, 85, 86, 88, 90, 91, 92, 93, 94, 95, 96, 97, 98, 99, 100, 101, 102, 103, 104, 105, 106, 107, 108, 109, 110, 111, 112, 113, 115, 117, 119, 120, 121, 122, 123, 124, 126, 128, 129, 131, 133, 135, 137, 139, 140, 141, 142, 144, 146, 147, 148, 149, 150, 152, 154, 155, 157, 158, 160, 162, 163, 165, 167, 169, 171, 173, 175, 176, 178, 180, 182, 183, 185, 187, 189, 191, 193, 194, 195, 196, 197, 198, 199, 200, 201, 203, 205, 207, 208, 209, 211, 213, 214, 215, 217, 218, 219, 220, 222, 223, 224, 226, 228, 229, 231, 232, 233, 234, 235, 236, 237, 238, 239, 240, 242, 244, 245, 246, 247, 248, 249, 250, 251, 252, 254, 255, 256, 257, 258, 259, 260, 261, 262, 263, 264, 265, 266, 267, 268, 269, 270, 271, 272, 273, 274, 275, 276, 277, 278, 279, 281, 282, 284, 286, 287, 288, 289, 291, 293, 294, 295, 297, 299, 300, 302, 304, 306, 307, 308, 310, 311, 313, 315, 316, 317, 318, 319, 320, 322, 324, 326, 328, 330, 331, 333, 335, 337, 338, 340, 342, 343, 345, 346, 347, 349, 350, 351, 353, 354, 355, 357, 358, 360, 362, 363, 364, 365, 367, 369, 370, 371, 372, 373, 374, 375, 377, 378, 379, 380, 382, 383, 384, 385, 386, 387, 389, 390, 391, 393, 395, 396, 398, 400, 401, 403, 404, 406, 408, 409, 410, 411, 412, 414, 415, 417, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 456, 457, 459, 461, 462, 464, 466, 467, 468, 469, 470, 472, 474, 475, 477, 479, 480, 482, 483, 484, 485, 487, 488, 489, 490, 491, 492, 493, 494, 495, 497, 499, 501, 502, 503, 504, 506, 507, 509, 510, 511, 512, 514, 515, 516, 517, 518, 520, 522, 524, 526, 528, 530, 531, 532, 533, 534, 535, 536, 537, 538, 539, 540, 542, 543, 545, 546, 547, 548, 550, 551, 553, 554, 555, 557, 559, 560, 562, 563, 565, 566, 568, 570, 572, 573, 575, 576, 577, 579, 580, 581, 582, 583, 584, 585, 586, 587, 589, 590, 591, 592, 593, 595, 596, 597, 598, 599, 601, 602, 604, 605, 607, 609, 610, 611, 612, 613, 614, 615, 616, 617, 618, 619, 620, 621, 622, 623, 624, 625, 626, 627, 629, 631, 633, 634, 635, 36, 637, 638, 640, 642, 644, 646, 648, 650, 651, 653, 654, 655, 656, 657, 658, 659, 660, 662, 663, 664, 665, 666, 667, 669, 671, 672, 674, 676, 677, 679, 680, 682, 684, 685, 687, 689, 690, 691, 693, 694, 696, 697, 698, 00, 702, 704, 705, 706, 707, 709, 710, 712, 714, 715, 717, 718, 719, 720, 721, 722, 723, 725, 726, 727, 728, 730, 731, 732, 733, 734, 735, 737, 739, 741, 743, 744, 745, 746, 747, 749, 751, 752, 753, 755, 757, 759, 760, 762, 764, 766, 768, 770, 772, 773, 775, 776, 778, 780, 782, 784, 786, 788, 789, 791, 793, 794, 795, 796, 798, 800, 801, 802, 804, 805, 807, 809, 811, 813, 815, 817, 819, 820, 822, 823, 824, 825, 827, 828, 830, 832, 834, 836, 838, 840, 842, 843, 844, 846, 848, 849, 850, 851, 853, 855, 856, 857, 858, 860, 861, 863, 864, 866, 868, 870, 871, 873, 874, 876, 878, 880, 882, 883, 885, 887, 889, 890, 892, 893, 895, 896, 897, 899, 901, 902, 903, 905, 907, 908, 909, 910, 912, 914, 915, 916, 917, 919, 921, 923, 925, 926, 928, 930, 932, 934, 935, 936, 937, 939, 941, 943, 945, 947, 949, 950, 952, 953, 955, 957, 959, 960, 962, 964, 965, 967, 968, 970, 972, 974, 976, 978, 980, 981, 983, 985, 86, 87, 989, 991, 993, 995, 997, 998, 1000, 1002, 1003, 1005, 1007, 1008, 1009, 1011, 1013, 1014, 1016, 1018, 1020, 1022, 1023, 1024, 1025, 1026, 1027, 1029, 1031, 1033, 1034, 1036, 1038, 1039, 1041, 1042, 1043, 1044, 1045, 1046, 1048, 1049, 1050, 1051, 1052, 1054, 1056, 1058, 1060, 1061, 1062, 1064, 1066, 1067, 1069, 1070, 1071, 1072, 1074, 1076, 1077, 1078, 1079, 1081, 1083, 1085, 1087, 1088, 1090, 1092, 1093, 1095, 1096, 1097, 1099, 1101, 1102, 1104, 1105, 1107, 1108, 1110, 1111, 1112, 1113, 1114, 1115, 1117, 1119, 1120, 1121, 1122, 1124, 1126, 1128, 1130, 1132, 1133, 1135, 1137, 1139, 1141, 1142, 1144, 1146, 1148, 1150, 1152, 1154, 1156, 1157, 1158, 1160, 1162, 1164, 1166, 1168, 1169, 1171, 1173, 1175, 1176, 1177, 1179, 1180, 1181, 1183, 1185, 1187, 1188, 1190, 1192, 1193, 1194, 1196, 1198, 1200, 1201, 1203, 1205, 1207, 1209, 1210, 1212, 1214, 1216, 1218, 1219, 1221, 1222, 1224, 1226, 1228, 1230, 1232, 1234, 1235, 1236, 1238, 1240, 1241, 1243, 1245, 1247, 1249, 1251, 1252, 1253, 1255, 1257, 1259, 1261, 1263, 1265, 1266, 1268, 1270, 1272, 1274, 1276, 1278, 1279, 1281, 1283, 1285, 1287, 1289, 1291, 1293, 1294, 1296, 1297, 1299, 1300, 1302, 1304, 1306, 1308, 1309, 1310, 1312, 1313, 1314, 1316, 1318, 1320, 1322, 1324, 1325, 1326, 1328, 1329, 1330, 1332, 1333, 1334, 1336, 1338, 1339, 1340, 1341, 1342, 1343, 1344, 1345, 1347, 1349, 1350, 1352, 1353, 1355, 1357, 1358, 1359, 1360, 1361, 1363, 1364, 1366, 1367, 1369, 1370, 1371, 1372, 1373, 1375, 1376, 1377, 1379, 1380, 1381, 1382, 1384, 1386, 1388, 1389, 1391, 1393, 1395, 1397, 1399, 1401, 1402, 1403, 1405, 1407, 1409, 1410, 1412, 1414, 1416, 1418, 1420, 1421, 1422, 1424, 1426, 1428, 1429, 1431, 1433, 1435, 1437, 1439, 1440, 1441, 1443, 1445, 1446, 1448, 1450, 1452, 1454, 1456, 1457, 1459, 1461, 1463, 1465, 1467, 1469, 1471, 1473, 1475, 1477, 1479, 1481, 1482, 1483, 1485, 1486, 1488, 1489, 1490, 1492, 1493, 1495, 1497, 1498, 1500, 1502, 1503, 1505, 1506, 1507, 1508, 1509, 1511, 1512, 1514, 1515, 1516, 1518, 1520, 1522, 1523, 1524, 1525, 1526, 1528, 1529, 1531, 1533, 1534, 1535, 1537, 1538, 1540, 1542, 1544, 1546, 1547, 1549, 1550, 1552, 1554, 1555, 1557, 1558, 1560, 1562, 1563, 1564, 1565, 1568, 1569, 1571, 1572, 1574, 1575, 1577, 1578, 1580, 1581, 1583, 1584, 1586, 1588, 1589, 1591, 1593, 1594, 1595, 1596, 1598, 1599, 1600, 1602, 1603, 1604, 1605, 1607, 1609, 1611, 1612, 1613, 1615, 1616, 1618, 1619, 1621, 1622, 1624, 1626, 1628, 1629, 1630, 1632, 1634, 1636, 1637, 1639, 1640, 1642, 1644, 1646, 1647, 1648, 1649, 1650, 1652, 1654, 1656, 1657, 1658, 1660, 1661, 1662, 1664, 1666, 1668, 1669, 1670, 1671, 1672, 1674, 1676, 1677, 1679, 1680, 1681, 1682, 1683, 1684, 1686, 1688, 1689, 1691, 1693, 1695, 1697, 1699, 1700, 1701, 1702, 1704, 1705, 1706, 1707, 1708, 1710, 1711, 1712, 1713, 1714, 1715, 1717, 1719, 1721, 1723, 1724, 1725, 1727, 1729, 1731, 1732, 1734, 1735, 1737, 1739, 1741, 1743, 1745, 1746, 1747, 1749, 1751, 1753, 1755, 1757, 1758, 1759, 1761, 1762, 1764, 1765, 1766, 1767, 1769, 1770, 1772, 1773, 1774, 1776, 1777, 1779, 1781, 1783, 1784, 1786, 1788, 1790, 1792, 1794, 1795, 1797, 798, 1799, 1801, 1803, 1804, 1805, 1806, 1807, 1808, 1809, 1810, 1811, 1812, 1813, 1815, 1816, 1817, 1819, 1821, 1822, 1823, 1825, 1826, 1827, 1828, 1829, 1831, 1833, 1834, 1835, 1837, 1839, 1841, 1842, 1844, 1845, 1847, 1849, 1850, 1852, 1853, 1854, 1855, 1856, 1857, 1858, 1859, 1861, 1863, 1865, 1866, 1867, 1869, 1871, 1872, 1874, 1876, 1877, 1879, 1881, 1882, 1883, 1885, 1887, 1889, 1891, 1893, 1894, 1895, 1897, 1898, 1900, 1902, 1903, 1904, 1906, 1908, 1910, 1912, 1913, 1915, 1917, 1918, 1919, 1920, 1922, 1924, 1926, 1928, 1929, 1931, 1933, 1935, 1936, 1938, 1940, 1942, 1944, 1946, 1948, 1949, 1951, 1953, 1955, 1957, 1959, 1961, 1963, 1965, 1967, 1969, 1971, 1972, 1973, 1974, 1975, 1976, 1978, 1979, 1980, 1982, 1983, 1985, 1986, 1987, 1989, 1990, 1992, 1994, 1996, 1997, 1999, 2000, 2002, 2003, 2005, 2007, 2008, 2009, 2010, 2011, 2013, 2015, 2016, 2017, 2018, 2019, 2021, 2022, 2023, 2024, 2026, 2028, 2030, 2031, 2032, 2034, 2036, 2037, 2038, 2040, 2041, 2042, 2043, 2044, 2046, 2047, 2048, 2049, 2051, 2053, 2055, 2056, 2058, 2060, 2062, 2064, 2066, 2067, 2069, 2070, 2072, 2073, 2074, 2075, 2076, 2077, 2078, 2079, 2081, 2083, 2085, 2087, 2088, 2090, 2091, 2092, 2093, 2095, 2097, 2098, 2100, 2102, 2104, 2105, 2106, 2108, 2110, 2112, 2113, 2115, 2117, 2118, 2119, 2120, 2121, 2122, 2124, 2126, 2128, 2129, 2130, 2132, 2133, 2135, 2136, 2137, 2138, 2140, 2141, 2142, 2143, 2144, 2146, 2148, 2149, 2150, 2151, 2152, 2153, 2154, 2156, 2158, 2159, 2160, 2161, 2162, 2163, 2165, 2167, 2169, 2170, 2171, 2172, 2173, 2174, 2175, 2176, 2177, 2178, 2179, 2181, 2183, 2184, 2186, 2187, 2188, 2190, 2192, 2194, 2195, 2196, 2197, 2198, 2199, 2200, 2201, 2202, 2203, 2205, 2207, 2209, 2211, 2213, 2215, 2217, 2219, 2221, 2223, 2225, 2226, 2228, 2229, 2231, 2233, 2235, 2236, 2237, 2238, 2239, 2240, 2241, 2243, 2244, 2246, 2248, 2250, 2251, 2253, 2255, 2257, 2259, 2261, 2262, 2264, 2265, 2267, 2269, 2270, 2272, 2273, 2275, 2276, 2278, 2279, 2281, 2282, 2284, 2285, 2286, 2288, 2289, 2291, 2293, 2295, 2296, 2297, 2298, 2299, 2300, 2302, 2304, 2306, 2307, 2309, 2310, 2312, 2313, 2314, 2315, 2316, 2317, 2318, 2320, 2321, 2323, 2325, 2327, 2329, 2330, 2332, 2334, 2335, 2337, 2339, 2341, 2343, 2345, 2346, 2347, 2348, 2350, 2352, 2354, 2356, 2357, 2358, 2360, 2361, 2362, 2364, 2366, 2367, 2369, 2371, 2373, 2375, 2376, 2378, 2380, 2381, 2383, 2384, 2386, 2388, 2390, 2391, 2392, 2394, 2396, 2397, 2399, 2401, 2402, 2403, 2405, 2407, 2408, 2409, 2411, 2413, 2415, 2417, 2418, 2419, 2421, 2422, 2423, 2424, 2425, 2427, 2428, 2429, 2430, 2432, 2434, 2435, 2437, 2439, 2440, 2441, 2442, 2443, 2445, 2446, 2448, 2449, 2451, 2452, 2454, 2456, 2457, 2458, 2459, 2460, 2461, 2462, 2463, 2464, 2466, 2468, 2469, 2470, 2471, 2472, 2474, 2475, 2476, 2477, 2478, 2480, 2482, 2483, 2484, 2485, 2486, 2487, 2488, 2489, 2490, 2491, 2493, 2494, 2495, 2496, 2497, 2498, 2499, 2500, 2502, 2503, 2505, 2506, 2507, 2509, 2510, 2511, 2512, 2513, 2514, 2516, 2517, 2518, 2519, 2521, 2522, 2523, 2525, 2527, 2529, 2530, 2531, 2533, 2535, 2537, 2539, 2540, 2541, 2542, 2544, 2546, 2547, 2548, 2550, 2551, 2552, 2554, 2556, 2557, 2558, 2559, 2561, 2562, 2564, 2565, 2567, 2569, 2571, 2572, 2573, 2574, 2575, 2576, 2578, 2580, 2581, 2583, 2584, 2585, 2587, 2588, 2589, 2590, 2592, 2593, 2594, 2595, 2597, 2599, 2601, 2603, 2604, 2606, 2608, 2609, 2611, 2612, 2614, 2615, 2616, 2617, 2619, 2621, 2623, 2624, 2626, 2627, 2628, 2629, 2631, 2632, 2634, 2636, 2638, 2640, 2641, 2642, 2644, 2646, 2647, 2648, 2649, 2650, 2652, 2654, 2656, 2658, 2659, 2661, 2663, 2665, 2667, 2668, 2670, 2672, 2674, 2675, 2677, 2679, 2681, 2683, 2685, 2687, 2689, 2691, 2693, 2695, 2697, 2699, 2701, 2703, 2704, 2706, 2708, 2710, 2711, 2712, 2714, 2716, 2717, 2719, 2721, 2722, 2724, 2726, 2728, 2730, 2732, 2734, 2735, 2736, 2738, 2739, 2740, 2741, 2743, 2745, 2746, 2748, 2750, 2751, 2753, 2754, 2756, 2758, 2760, 2762, 2764, 2766, 2768, 2770, 2771, 2772, 2773, 2775, 2777, 2778, 2780, 2781, 2782, 2784, 2786, 2788, 2790, 2791, 2792, 2794, 2796, 2797, 2799, 2801, 2802, 2803, 2804, 2806, 2807, 2809, 2810, 2811, 2813, 2815, 2816, 2818, 2819, 2821, 2822, 2823, 2825, 2826, 2828, 2830, 2832, 2833, 2835, 2836, 2838, 2840, 2842, 2844, 2845, 2846, 2847, 2849, 2850, 2851, 2852, 2853, 2855, 2856, 2857, 2858, 2859, 2861, 2862, 2863, 2864, 2866, 2867, 2868, 2869, 2870, 2872, 2873, 2875, 2876, 2877, 2878, 2880, 2882, 2883, 2885, 2886, 2888, 2890, 2891, 2892, 2893, 2894, 2896, 2898, 2900, 2902, 2903, 2904, 2906, 2907, 2908, 2909, 2910, 2911, 2915, 2917, 2918, 2919, 2920, 2921, 2922, 2923, 2924, or 2926. In another aspect, a method comprises introducing into a plant cell an exogenous nucleic acid, that comprises a regulatory region operably linked to a nucleotide sequence having 80 percent or greater sequence identity to a nucleotide sequence set forth in SEQ ID NO:1, 3, 7, 9, 11, 14, 16, 19, 21, 23, 28, 31, 33, 36, 40, 42, 44, 46, 48, 50, 52, 54, 58, 61, 66, 68, 72, 76, 78, 87, 89, 114, 116, 118, 125, 127, 130, 132, 134, 136, 138, 143, 145, 151, 153, 156, 159, 161, 164, 166, 168, 170, 172, 174, 177, 179, 181, 184, 186, 188, 190, 192, 202, 204, 206, 210, 212, 216, 221, 225, 227, 230, 241, 243, 253, 280, 283, 285, 290, 292, 296, 298, 301, 303, 305, 309, 312, 314, 321, 323, 325, 327, 329, 332, 334, 336, 339, 341, 344, 348, 352, 356, 359, 361, 366, 368, 376, 381, 388, 392, 394, 397, 399, 402, 405, 407, 413, 416, 418, 455, 458, 460, 463, 465, 471, 473, 476, 478, 481, 486, 496, 498, 500, 505, 508, 513, 519, 521, 523, 525, 527, 529, 541, 544, 549, 552, 556, 558, 561, 564, 567, 569, 571, 574, 578, 588, 594, 600, 603, 606, 608, 628, 630, 632, 639, 641, 643, 645, 647, 649, 652, 661, 668, 670, 673, 675, 678, 681, 683, 686, 688, 692, 695, 699, 701, 703, 708, 711, 713, 716, 724, 729, 736, 738, 740, 742, 748, 750, 754, 756, 758, 761, 763, 765, 767, 769, 771, 774, 777, 779, 781, 783, 785, 787, 790, 792, 797, 799, 803, 806, 808, 810, 812, 814, 816, 818, 821, 826, 829, 831, 833, 835, 837, 839, 841, 845, 847, 852, 854, 859, 862, 865, 867, 869, 872, 875, 877, 879, 881, 884, 886, 888, 891, 894, 898, 900, 904, 906, 911, 913, 918, 920, 922, 924, 927, 929, 931, 933, 938, 940, 942, 944, 946, 948, 951, 954, 956, 958, 961, 963, 966, 969, 971, 973, 975, 977, 979, 982, 984, 988, 990, 992, 994, 996, 999, 1001, 1004, 1006, 1010, 1012, 1015, 1017, 1019, 1021, 1028, 1030, 1032, 1035, 1037, 1040, 1047, 1053, 1055, 1057, 1059, 1063, 1065, 1068, 1073, 1075, 1080, 1082, 1084, 1086, 1089, 1091, 1094, 1098, 1100, 1103, 1106, 1109, 1116, 1118, 1123, 1125, 1127, 1129, 1131, 1134, 1136, 1138, 1140, 1143, 1145, 1147, 1149, 1151, 1153, 1155, 1159, 1161, 1163, 1165, 1167, 1170, 1172, 1174, 1178, 1182, 1184, 1186, 1189, 1191, 1195, 1197, 1199, 1202, 1204, 1206, 1208, 1211, 1213, 1215, 1217, 1220, 1223, 1225, 1227, 1229, 1231, 1233, 1237, 1239, 1242, 1244, 1246, 1248, 1250, 1254, 1256, 1258, 1260, 1262, 1264, 1267, 1269, 1271, 1273, 1275, 1277, 1280, 1282, 1284, 1286, 1288, 1290, 1292, 1295, 1298, 1301, 1303, 1305, 1307, 1311, 1315, 1317, 1319, 1321, 1323, 1327, 1331, 1335, 1337, 1346, 1348, 1351, 1354, 1356, 1362, 1365, 1368, 1374, 1378, 1383, 1385, 1387, 1390, 1392, 1394, 1396, 1398, 1400, 1404, 1406, 1408, 1411, 1413, 1415, 1417, 1419, 1423, 1425, 1427, 1430, 1432, 1434, 1436, 1438, 1442, 1444, 1447, 1449, 1451, 1453, 1455, 1458, 1460, 1462, 1464, 1466, 1468, 1470, 1472, 1474, 1476, 1478, 1480, 1484, 1487, 1491, 1494, 1496, 1499, 1501, 1504, 1510, 1513, 1517, 1519, 1521, 1527, 1530, 1532, 1536, 1539, 1541, 1543, 1545, 1548, 1551, 1553, 1556, 1559, 1561, 1566, 1568, 1570, 1573, 1576, 1579, 1582, 1585, 1587, 1590, 1592, 1597, 1601, 1606, 1608, 1610, 1614, 1617, 1620, 1623, 1625, 1627, 1631, 1633, 1635, 1638, 1641, 1643, 1645, 1651, 1653, 1655, 1659, 1663, 1665, 1667, 1673, 1675, 1678, 1685, 1687, 1690, 1692, 1694, 1696, 1698, 1703, 1709, 1716, 1718, 1720, 1722, 1726, 1728, 1730, 1733, 1736, 1738, 1740, 1742, 1744, 1748, 1750, 1752, 1754, 1756, 1760, 1763, 1768, 1771, 1775, 1778, 1780, 1782, 1785, 1787, 1789, 1791, 1793, 1796, 1800, 1802, 1814, 1818, 1820, 1824, 1830, 1832, 1836, 1838, 1840, 1843, 1846, 1848, 1851, 1860, 1862, 1864, 1868, 1870, 1873, 1875, 1878, 1880, 1884, 1886, 1888, 1890, 1892, 1896, 1899, 1901, 1905, 1907, 1909, 1911, 1914, 1916, 1921, 1923, 1925, 1927, 1930, 1932, 1934, 1937, 1939, 1941, 1943, 1945, 1947, 1950, 1952, 1954, 1956, 1958, 1960, 1962, 1964, 1966, 1968, 1970, 1977, 1981, 1984, 1988, 1991, 1993, 1995, 1998, 2001, 2004, 2006, 2012, 2014, 2020, 2025, 2027, 2029, 2033, 2035, 2039, 2045, 2050, 2052, 2054, 2057, 2059, 2061, 2063, 2065, 2068, 2071, 2080, 2082, 2084, 2086, 2089, 2094, 2096, 2099, 2101, 2103, 2107, 2109, 2111, 2114, 2116, 2123, 2125, 2127, 2131, 2134, 2139, 2145, 2147, 2155, 2157, 2164, 2166, 2168, 2180, 2182, 2185, 2189, 2191, 2193, 2204, 2206, 2208, 2210, 2212, 2214, 2216, 2218, 2220, 2222, 2224, 2227, 2230, 2232, 2234, 2242, 2245, 2247, 2249, 2252, 2254, 2256, 2258, 2260, 2263, 2266, 2268, 2271, 2274, 2277, 2280, 2283, 2287, 2290, 2292, 2294, 2301, 2303, 2305, 2308, 2311, 2319, 2322, 2324, 2326, 2328, 2331, 2333, 2336, 2338, 2340, 2342, 2344, 2349, 2351, 2353, 2355, 2359, 2363, 2365, 2368, 2370, 2372, 2374, 2377, 2379, 2382, 2385, 2387, 2389, 2393, 2395, 2398, 2400, 2404, 2406, 2410, 2412, 2414, 2416, 2420, 2426, 2431, 2433, 2436, 2438, 2444, 2447, 2450, 2453, 2455, 2465, 2467, 2473, 2479, 2481, 2492, 2501, 2504, 2508, 2515, 2520, 2524, 2526, 2528, 2532, 2534, 2536, 2538, 2543, 2545, 2549, 2553, 2555, 2560, 2563, 2566, 2568, 2570, 2577, 2579, 2582, 2586, 2591, 2596, 2598, 2600, 2602, 2605, 2607, 2610, 2613, 2618, 2620, 2622, 2625, 2630, 2633, 2635, 2637, 2639, 2643, 2645, 2651, 2653, 2655, 2657, 2660, 2662, 2664, 2666, 2669, 2671, 2673, 2676, 2678, 2680, 2682, 2684, 2686, 2688, 2690, 2692, 2694, 2696, 2698, 2700, 2702, 2705, 2707, 2709, 2713, 2715, 2718, 2720, 2723, 2725, 2727, 2729, 2731, 2733, 2737, 2742, 2744, 2747, 2749, 2752, 2755, 2757, 2759, 2761, 2763, 2765, 2767, 2769, 2774, 2776, 2779, 2783, 2785, 2787, 2789, 2793, 2795, 2798, 2800, 2805, 2808, 2812, 2814, 2817, 2820, 2824, 2827, 2829, 2831, 2834, 2837, 2839, 2841, 2843, 2848, 2854, 2860, 2865, 2871, 2874, 2879, 2881, 2884, 2887, 2889, 2895, 2897, 2899, 2901, 2905, 2912, 2914, 2916, or 2925, or a fragment thereof. A plant produced from the plant cell has a difference in the level of biomass as compared to the corresponding level of biomass of a control plant that does not comprise the exogenous nucleic acid.

Plant cells comprising an exogenous nucleic acid are provided herein. In one aspect, the exogenous nucleic acid comprises a regulatory region operably linked to a nucleotide sequence encoding a polypeptide. The HMM bit score of the amino acid sequence of the polypeptide is greater than about 915, 1149, 618, 324, 752, 866, 703, 862, 883, 393, 232, 815, 1251, 1066, 1398, 370, 328, 133, 265, 843, 93, 175, 2105, 1213, 1174, 1138, 1126, 1812, 824, 1240, 1078, 873, 1102, 754, 539, 657, 1925, 1104, 1347, 1096, 851, 761, 815, 1121, 681, 677, 146, 286, 493, or 316, using an HMM based on the amino acid sequences depicted in one of FIG. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. The plant has a difference in the level of biomass as compared to the corresponding level of biomass of a control plant that does not comprise the exogenous nucleic acid. In another aspect, the exogenous nucleic acid comprises a regulatory region operably linked to a nucleotide sequence encoding a polypeptide having 80 percent or greater sequence identity to an amino acid sequence selected from the group consisting of SEQ ID NO:2, 4, 5, 6, 8, 10, 12, 13, 15, 17, 18, 20, 22, 24, 25, 26, 27, 29, 30, 32, 34, 35, 37, 38, 39, 41, 43, 45, 47, 49, 51, 53, 55, 56, 57, 59, 60, 62, 63, 64, 65, 67, 69, 70, 71, 73, 74, 75, 77, 79, 80, 81, 82, 83, 84, 85, 86, 88, 90, 91, 92, 93, 94, 95, 96, 97, 98, 99, 100, 101, 102, 103, 104, 105, 106, 107, 108, 109, 110, 111, 112, 113, 115, 117, 119, 120, 121, 122, 123, 124, 126, 128, 129, 131, 133, 135, 137, 139, 140, 141, 142, 144, 146, 147, 148, 149, 150, 152, 154, 155, 157, 158, 160, 162, 163, 165, 167, 169, 171, 173, 175, 176, 178, 180, 182, 183, 185, 187, 189, 191, 193, 194, 195, 196, 197, 198, 199, 200, 201, 203, 205, 207, 208, 209, 211, 213, 214, 215, 217, 218, 219, 220, 222, 223, 224, 226, 228, 229, 231, 232, 233, 234, 235, 236, 237, 238, 239, 240, 242, 244, 245, 246, 247, 248, 249, 250, 251, 252, 254, 255, 256, 257, 258, 259, 260, 261, 262, 263, 264, 265, 266, 267, 268, 269, 270, 271, 272, 273, 274, 275, 276, 277, 278, 279, 281, 282, 284, 286, 287, 288, 289, 291, 293, 294, 295, 297, 299, 300, 302, 304, 306, 307, 308, 310, 311, 313, 315, 316, 317, 318, 319, 320, 322, 324, 326, 328, 330, 331, 333, 335, 337, 338, 340, 342, 343, 345, 346, 347, 349, 350, 351, 353, 354, 355, 357, 358, 360, 362, 363, 364, 365, 367, 369, 370, 371, 372, 373, 374, 375, 377, 378, 379, 380, 382, 383, 384, 385, 386, 387, 389, 390, 391, 393, 395, 396, 398, 400, 401, 403, 404, 406, 408, 409, 410, 411, 412, 414, 415, 417, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 456, 457, 459, 461, 462, 464, 466, 467, 468, 469, 470, 472, 474, 475, 477, 479, 480, 482, 483, 484, 485, 487, 488, 489, 490, 491, 492, 493, 494, 495, 497, 499, 501, 502, 503, 504, 506, 507, 509, 510, 511, 512, 514, 515, 516, 517, 518, 520, 522, 524, 526, 528, 530, 531, 532, 533, 534, 535, 536, 537, 538, 539, 540, 542, 543, 545, 546, 547, 548, 550, 551, 553, 554, 555, 557, 559, 560, 562, 563, 565, 566, 568, 570, 572, 573, 575, 576, 577, 579, 580, 581, 582, 583, 584, 585, 586, 587, 589, 590, 591, 592, 593, 595, 596, 597, 598, 599, 601, 602, 604, 605, 607, 609, 610, 611, 612, 613, 614, 615, 616, 617, 618, 619, 620, 621, 622, 623, 624, 625, 626, 627, 629, 631, 633, 634, 635, 36, 637, 638, 640, 642, 644, 646, 648, 650, 651, 653, 654, 655, 656, 657, 658, 659, 660, 662, 663, 664, 665, 666, 667, 669, 671, 672, 674, 676, 677, 679, 680, 682, 684, 685, 687, 689, 690, 691, 693, 694, 696, 697, 698, 00, 702, 704, 705, 706, 707, 709, 710, 712, 714, 715, 717, 718, 719, 720, 721, 722, 723, 725, 726, 727, 728, 730, 731, 732, 733, 734, 735, 737, 739, 741, 743, 744, 745, 746, 747, 749, 751, 752, 753, 755, 757, 759, 760, 762, 764, 766, 768, 770, 772, 773, 775, 776, 778, 780, 782, 784, 786, 788, 789, 791, 793, 794, 795, 796, 798, 800, 801, 802, 804, 805, 807, 809, 811, 813, 815, 817, 819, 820, 822, 823, 824, 825, 827, 828, 830, 832, 834, 836, 838, 840, 842, 843, 844, 846, 848, 849, 850, 851, 853, 855, 856, 857, 858, 860, 861, 863, 864, 866, 868, 870, 871, 873, 874, 876, 878, 880, 882, 883, 885, 887, 889, 890, 892, 893, 895, 896, 897, 899, 901, 902, 903, 905, 907, 908, 909, 910, 912, 914, 915, 916, 917, 919, 921, 923, 925, 926, 928, 930, 932, 934, 935, 936, 937, 939, 941, 943, 945, 947, 949, 950, 952, 953, 955, 957, 959, 960, 962, 964, 965, 967, 968, 970, 972, 974, 976, 978, 980, 981, 983, 985, 86, 87, 989, 991, 993, 995, 997, 998, 1000, 1002, 1003, 1005, 1007, 1008, 1009, 1011, 1013, 1014, 1016, 1018, 1020, 1022, 1023, 1024, 1025, 1026, 1027, 1029, 1031, 1033, 1034, 1036, 1038, 1039, 1041, 1042, 1043, 1044, 1045, 1046, 1048, 1049, 1050, 1051, 1052, 1054, 1056, 1058, 1060, 1061, 1062, 1064, 1066, 1067, 1069, 1070, 1071, 1072, 1074, 1076, 1077, 1078, 1079, 1081, 1083, 1085, 1087, 1088, 1090, 1092, 1093, 1095, 1096, 1097, 1099, 1101, 1102, 1104, 1105, 1107, 1108, 1110, 1111, 1112, 1113, 1114, 1115, 1117, 1119, 1120, 1121, 1122, 1124, 1126, 1128, 1130, 1132, 1133, 1135, 1137, 1139, 1141, 1142, 1144, 1146, 1148, 1150, 1152, 1154, 1156, 1157, 1158, 1160, 1162, 1164, 1166, 1168, 1169, 1171, 1173, 1175, 1176, 1177, 1179, 1180, 1181, 1183, 1185, 1187, 1188, 1190, 1192, 1193, 1194, 1196, 1198, 1200, 1201, 1203, 1205, 1207, 1209, 1210, 1212, 1214, 1216, 1218, 1219, 1221, 1222, 1224, 1226, 1228, 1230, 1232, 1234, 1235, 1236, 1238, 1240, 1241, 1243, 1245, 1247, 1249, 1251, 1252, 1253, 1255, 1257, 1259, 1261, 1263, 1265, 1266, 1268, 1270, 1272, 1274, 1276, 1278, 1279, 1281, 1283, 1285, 1287, 1289, 1291, 1293, 1294, 1296, 1297, 1299, 1300, 1302, 1304, 1306, 1308, 1309, 1310, 1312, 1313, 1314, 1316, 1318, 1320, 1322, 1324, 1325, 1326, 1328, 1329, 1330, 1332, 1333, 1334, 1336, 1338, 1339, 1340, 1341, 1342, 1343, 1344, 1345, 1347, 1349, 1350, 1352, 1353, 1355, 1357, 1358, 1359, 1360, 1361, 1363, 1364, 1366, 1367, 1369, 1370, 1371, 1372, 1373, 1375, 1376, 1377, 1379, 1380, 1381, 1382, 1384, 1386, 1388, 1389, 1391, 1393, 1395, 1397, 1399, 1401, 1402, 1403, 1405, 1407, 1409, 1410, 1412, 1414, 1416, 1418, 1420, 1421, 1422, 1424, 1426, 1428, 1429, 1431, 1433, 1435, 1437, 1439, 1440, 1441, 1443, 1445, 1446, 1448, 1450, 1452, 1454, 1456, 1457, 1459, 1461, 1463, 1465, 1467, 1469, 1471, 1473, 1475, 1477, 1479, 1481, 1482, 1483, 1485, 1486, 1488, 1489, 1490, 1492, 1493, 1495, 1497, 1498, 1500, 1502, 1503, 1505, 1506, 1507, 1508, 1509, 1511, 1512, 1514, 1515, 1516, 1518, 1520, 1522, 1523, 1524, 1525, 1526, 1528, 1529, 1531, 1533, 1534, 1535, 1537, 1538, 1540, 1542, 1544, 1546, 1547, 1549, 1550, 1552, 1554, 1555, 1557, 1558, 1560, 1562, 1563, 1564, 1565, 1568, 1569, 1571, 1572, 1574, 1575, 1577, 1578, 1580, 1581, 1583, 1584, 1586, 1588, 1589, 1591, 1593, 1594, 1595, 1596, 1598, 1599, 1600, 1602, 1603, 1604, 1605, 1607, 1609, 1611, 1612, 1613, 1615, 1616, 1618, 1619, 1621, 1622, 1624, 1626, 1628, 1629, 1630, 1632, 1634, 1636, 1637, 1639, 1640, 1642, 1644, 1646, 1647, 1648, 1649, 1650, 1652, 1654, 1656, 1657, 1658, 1660, 1661, 1662, 1664, 1666, 1668, 1669, 1670, 1671, 1672, 1674, 1676, 1677, 1679, 1680, 1681, 1682, 1683, 1684, 1686, 1688, 1689, 1691, 1693, 1695, 1697, 1699, 1700, 1701, 1702, 1704, 1705, 1706, 1707, 1708, 1710, 1711, 1712, 1713, 1714, 1715, 1717, 1719, 1721, 1723, 1724, 1725, 1727, 1729, 1731, 1732, 1734, 1735, 1737, 1739, 1741, 1743, 1745, 1746, 1747, 1749, 1751, 1753, 1755, 1757, 1758, 1759, 1761, 1762, 1764, 1765, 1766, 1767, 1769, 1770, 1772, 1773, 1774, 1776, 1777, 1779, 1781, 1783, 1784, 1786, 1788, 1790, 1792, 1794, 1795, 1797, 798, 1799, 1801, 1803, 1804, 1805, 1806, 1807, 1808, 1809, 1810, 1811, 1812, 1813, 1815, 1816, 1817, 1819, 1821, 1822, 1823, 1825, 1826, 1827, 1828, 1829, 1831, 1833, 1834, 1835, 1837, 1839, 1841, 1842, 1844, 1845, 1847, 1849, 1850, 1852, 1853, 1854, 1855, 1856, 1857, 1858, 1859, 1861, 1863, 1865, 1866, 1867, 1869, 1871, 1872, 1874, 1876, 1877, 1879, 1881, 1882, 1883, 1885, 1887, 1889, 1891, 1893, 1894, 1895, 1897, 1898, 1900, 1902, 1903, 1904, 1906, 1908, 1910, 1912, 1913, 1915, 1917, 1918, 1919, 1920, 1922, 1924, 1926, 1928, 1929, 1931, 1933, 1935, 1936, 1938, 1940, 1942, 1944, 1946, 1948, 1949, 1951, 1953, 1955, 1957, 1959, 1961, 1963, 1965, 1967, 1969, 1971, 1972, 1973, 1974, 1975, 1976, 1978, 1979, 1980, 1982, 1983, 1985, 1986, 1987, 1989, 1990, 1992, 1994, 1996, 1997, 1999, 2000, 2002, 2003, 2005, 2007, 2008, 2009, 2010, 2011, 2013, 2015, 2016, 2017, 2018, 2019, 2021, 2022, 2023, 2024, 2026, 2028, 2030, 2031, 2032, 2034, 2036, 2037, 2038, 2040, 2041, 2042, 2043, 2044, 2046, 2047, 2048, 2049, 2051, 2053, 2055, 2056, 2058, 2060, 2062, 2064, 2066, 2067, 2069, 2070, 2072, 2073, 2074, 2075, 2076, 2077, 2078, 2079, 2081, 2083, 2085, 2087, 2088, 2090, 2091, 2092, 2093, 2095, 2097, 2098, 2100, 2102, 2104, 2105, 2106, 2108, 2110, 2112, 2113, 2115, 2117, 2118, 2119, 2120, 2121, 2122, 2124, 2126, 2128, 2129, 2130, 2132, 2133, 2135, 2136, 2137, 2138, 2140, 2141, 2142, 2143, 2144, 2146, 2148, 2149, 2150, 2151, 2152, 2153, 2154, 2156, 2158, 2159, 2160, 2161, 2162, 2163, 2165, 2167, 2169, 2170, 2171, 2172, 2173, 2174, 2175, 2176, 2177, 2178, 2179, 2181, 2183, 2184, 2186, 2187, 2188, 2190, 2192, 2194, 2195, 2196, 2197, 2198, 2199, 2200, 2201, 2202, 2203, 2205, 2207, 2209, 2211, 2213, 2215, 2217, 2219, 2221, 2223, 2225, 2226, 2228, 2229, 2231, 2233, 2235, 2236, 2237, 2238, 2239, 2240, 2241, 2243, 2244, 2246, 2248, 2250, 2251, 2253, 2255, 2257, 2259, 2261, 2262, 2264, 2265, 2267, 2269, 2270, 2272, 2273, 2275, 2276, 2278, 2279, 2281, 2282, 2284, 2285, 2286, 2288, 2289, 2291, 2293, 2295, 2296, 2297, 2298, 2299, 2300, 2302, 2304, 2306, 2307, 2309, 2310, 2312, 2313, 2314, 2315, 2316, 2317, 2318, 2320, 2321, 2323, 2325, 2327, 2329, 2330, 2332, 2334, 2335, 2337, 2339, 2341, 2343, 2345, 2346, 2347, 2348, 2350, 2352, 2354, 2356, 2357, 2358, 2360, 2361, 2362, 2364, 2366, 2367, 2369, 2371, 2373, 2375, 2376, 2378, 2380, 2381, 2383, 2384, 2386, 2388, 2390, 2391, 2392, 2394, 2396, 2397, 2399, 2401, 2402, 2403, 2405, 2407, 2408, 2409, 2411, 2413, 2415, 2417, 2418, 2419, 2421, 2422, 2423, 2424, 2425, 2427, 2428, 2429, 2430, 2432, 2434, 2435, 2437, 2439, 2440, 2441, 2442, 2443, 2445, 2446, 2448, 2449, 2451, 2452, 2454, 2456, 2457, 2458, 2459, 2460, 2461, 2462, 2463, 2464, 2466, 2468, 2469, 2470, 2471, 2472, 2474, 2475, 2476, 2477, 2478, 2480, 2482, 2483, 2484, 2485, 2486, 2487, 2488, 2489, 2490, 2491, 2493, 2494, 2495, 2496, 2497, 2498, 2499, 2500, 2502, 2503, 2505, 2506, 2507, 2509, 2510, 2511, 2512, 2513, 2514, 2516, 2517, 2518, 2519, 2521, 2522, 2523, 2525, 2527, 2529, 2530, 2531, 2533, 2535, 2537, 2539, 2540, 2541, 2542, 2544, 2546, 2547, 2548, 2550, 2551, 2552, 2554, 2556, 2557, 2558, 2559, 2561, 2562, 2564, 2565, 2567, 2569, 2571, 2572, 2573, 2574, 2575, 2576, 2578, 2580, 2581, 2583, 2584, 2585, 2587, 2588, 2589, 2590, 2592, 2593, 2594, 2595, 2597, 2599, 2601, 2603, 2604, 2606, 2608, 2609, 2611, 2612, 2614, 2615, 2616, 2617, 2619, 2621, 2623, 2624, 2626, 2627, 2628, 2629, 2631, 2632, 2634, 2636, 2638, 2640, 2641, 2642, 2644, 2646, 2647, 2648, 2649, 2650, 2652, 2654, 2656, 2658, 2659, 2661, 2663, 2665, 2667, 2668, 2670, 2672, 2674, 2675, 2677, 2679, 2681, 2683, 2685, 2687, 2689, 2691, 2693, 2695, 2697, 2699, 2701, 2703, 2704, 2706, 2708, 2710, 2711, 2712, 2714, 2716, 2717, 2719, 2721, 2722, 2724, 2726, 2728, 2730, 2732, 2734, 2735, 2736, 2738, 2739, 2740, 2741, 2743, 2745, 2746, 2748, 2750, 2751, 2753, 2754, 2756, 2758, 2760, 2762, 2764, 2766, 2768, 2770, 2771, 2772, 2773, 2775, 2777, 2778, 2780, 2781, 2782, 2784, 2786, 2788, 2790, 2791, 2792, 2794, 2796, 2797, 2799, 2801, 2802, 2803, 2804, 2806, 2807, 2809, 2810, 2811, 2813, 2815, 2816, 2818, 2819, 2821, 2822, 2823, 2825, 2826, 2828, 2830, 2832, 2833, 2835, 2836, 2838, 2840, 2842, 2844, 2845, 2846, 2847, 2849, 2850, 2851, 2852, 2853, 2855, 2856, 2857, 2858, 2859, 2861, 2862, 2863, 2864, 2866, 2867, 2868, 2869, 2870, 2872, 2873, 2875, 2876, 2877, 2878, 2880, 2882, 2883, 2885, 2886, 2888, 2890, 2891, 2892, 2893, 2894, 2896, 2898, 2900, 2902, 2903, 2904, 2906, 2907, 2908, 2909, 2910, 2911, 2915, 2917, 2918, 2919, 2920, 2921, 2922, 2923, 2924, or 2926. A plant produced from the plant cell has a difference in the level of biomass as compared to the corresponding level of biomass of a control plant that does not comprise the exogenous nucleic acid. In another aspect, the exogenous nucleic acid comprises a regulatory region operably linked to a nucleotide sequence having 80 percent or greater sequence identity to a nucleotide sequence selected from the group consisting of SEQ ID NO:1, 3, 7, 9, 11, 14, 16, 19, 21, 23, 28, 31, 33, 36, 40, 42, 44, 46, 48, 50, 52, 54, 58, 61, 66, 68, 72, 76, 78, 87, 89, 114, 116, 118, 125, 127, 130, 132, 134, 136, 138, 143, 145, 151, 153, 156, 159, 161, 164, 166, 168, 170, 172, 174, 177, 179, 181, 184, 186, 188, 190, 192, 202, 204, 206, 210, 212, 216, 221, 225, 227, 230, 241, 243, 253, 280, 283, 285, 290, 292, 296, 298, 301, 303, 305, 309, 312, 314, 321, 323, 325, 327, 329, 332, 334, 336, 339, 341, 344, 348, 352, 356, 359, 361, 366, 368, 376, 381, 388, 392, 394, 397, 399, 402, 405, 407, 413, 416, 418, 455, 458, 460, 463, 465, 471, 473, 476, 478, 481, 486, 496, 498, 500, 505, 508, 513, 519, 521, 523, 525, 527, 529, 541, 544, 549, 552, 556, 558, 561, 564, 567, 569, 571, 574, 578, 588, 594, 600, 603, 606, 608, 628, 630, 632, 639, 641, 643, 645, 647, 649, 652, 661, 668, 670, 673, 675, 678, 681, 683, 686, 688, 692, 695, 699, 701, 703, 708, 711, 713, 716, 724, 729, 736, 738, 740, 742, 748, 750, 754, 756, 758, 761, 763, 765, 767, 769, 771, 774, 777, 779, 781, 783, 785, 787, 790, 792, 797, 799, 803, 806, 808, 810, 812, 814, 816, 818, 821, 826, 829, 831, 833, 835, 837, 839, 841, 845, 847, 852, 854, 859, 862, 865, 867, 869, 872, 875, 877, 879, 881, 884, 886, 888, 891, 894, 898, 900, 904, 906, 911, 913, 918, 920, 922, 924, 927, 929, 931, 933, 938, 940, 942, 944, 946, 948, 951, 954, 956, 958, 961, 963, 966, 969, 971, 973, 975, 977, 979, 982, 984, 988, 990, 992, 994, 996, 999, 1001, 1004, 1006, 1010, 1012, 1015, 1017, 1019, 1021, 1028, 1030, 1032, 1035, 1037, 1040, 1047, 1053, 1055, 1057, 1059, 1063, 1065, 1068, 1073, 1075, 1080, 1082, 1084, 1086, 1089, 1091, 1094, 1098, 1100, 1103, 1106, 1109, 1116, 1118, 1123, 1125, 1127, 1129, 1131, 1134, 1136, 1138, 1140, 1143, 1145, 1147, 1149, 1151, 1153, 1155, 1159, 1161, 1163, 1165, 1167, 1170, 1172, 1174, 1178, 1182, 1184, 1186, 1189, 1191, 1195, 1197, 1199, 1202, 1204, 1206, 1208, 1211, 1213, 1215, 1217, 1220, 1223, 1225, 1227, 1229, 1231, 1233, 1237, 1239, 1242, 1244, 1246, 1248, 1250, 1254, 1256, 1258, 1260, 1262, 1264, 1267, 1269, 1271, 1273, 1275, 1277, 1280, 1282, 1284, 1286, 1288, 1290, 1292, 1295, 1298, 1301, 1303, 1305, 1307, 1311, 1315, 1317, 1319, 1321, 1323, 1327, 1331, 1335, 1337, 1346, 1348, 1351, 1354, 1356, 1362, 1365, 1368, 1374, 1378, 1383, 1385, 1387, 1390, 1392, 1394, 1396, 1398, 1400, 1404, 1406, 1408, 1411, 1413, 1415, 1417, 1419, 1423, 1425, 1427, 1430, 1432, 1434, 1436, 1438, 1442, 1444, 1447, 1449, 1451, 1453, 1455, 1458, 1460, 1462, 1464, 1466, 1468, 1470, 1472, 1474, 1476, 1478, 1480, 1484, 1487, 1491, 1494, 1496, 1499, 1501, 1504, 1510, 1513, 1517, 1519, 1521, 1527, 1530, 1532, 1536, 1539, 1541, 1543, 1545, 1548, 1551, 1553, 1556, 1559, 1561, 1566, 1568, 1570, 1573, 1576, 1579, 1582, 1585, 1587, 1590, 1592, 1597, 1601, 1606, 1608, 1610, 1614, 1617, 1620, 1623, 1625, 1627, 1631, 1633, 1635, 1638, 1641, 1643, 1645, 1651, 1653, 1655, 1659, 1663, 1665, 1667, 1673, 1675, 1678, 1685, 1687, 1690, 1692, 1694, 1696, 1698, 1703, 1709, 1716, 1718, 1720, 1722, 1726, 1728, 1730, 1733, 1736, 1738, 1740, 1742, 1744, 1748, 1750, 1752, 1754, 1756, 1760, 1763, 1768, 1771, 1775, 1778, 1780, 1782, 1785, 1787, 1789, 1791, 1793, 1796, 1800, 1802, 1814, 1818, 1820, 1824, 1830, 1832, 1836, 1838, 1840, 1843, 1846, 1848, 1851, 1860, 1862, 1864, 1868, 1870, 1873, 1875, 1878, 1880, 1884, 1886, 1888, 1890, 1892, 1896, 1899, 1901, 1905, 1907, 1909, 1911, 1914, 1916, 1921, 1923, 1925, 1927, 1930, 1932, 1934, 1937, 1939, 1941, 1943, 1945, 1947, 1950, 1952, 1954, 1956, 1958, 1960, 1962, 1964, 1966, 1968, 1970, 1977, 1981, 1984, 1988, 1991, 1993, 1995, 1998, 2001, 2004, 2006, 2012, 2014, 2020, 2025, 2027, 2029, 2033, 2035, 2039, 2045, 2050, 2052, 2054, 2057, 2059, 2061, 2063, 2065, 2068, 2071, 2080, 2082, 2084, 2086, 2089, 2094, 2096, 2099, 2101, 2103, 2107, 2109, 2111, 2114, 2116, 2123, 2125, 2127, 2131, 2134, 2139, 2145, 2147, 2155, 2157, 2164, 2166, 2168, 2180, 2182, 2185, 2189, 2191, 2193, 2204, 2206, 2208, 2210, 2212, 2214, 2216, 2218, 2220, 2222, 2224, 2227, 2230, 2232, 2234, 2242, 2245, 2247, 2249, 2252, 2254, 2256, 2258, 2260, 2263, 2266, 2268, 2271, 2274, 2277, 2280, 2283, 2287, 2290, 2292, 2294, 2301, 2303, 2305, 2308, 2311, 2319, 2322, 2324, 2326, 2328, 2331, 2333, 2336, 2338, 2340, 2342, 2344, 2349, 2351, 2353, 2355, 2359, 2363, 2365, 2368, 2370, 2372, 2374, 2377, 2379, 2382, 2385, 2387, 2389, 2393, 2395, 2398, 2400, 2404, 2406, 2410, 2412, 2414, 2416, 2420, 2426, 2431, 2433, 2436, 2438, 2444, 2447, 2450, 2453, 2455, 2465, 2467, 2473, 2479, 2481, 2492, 2501, 2504, 2508, 2515, 2520, 2524, 2526, 2528, 2532, 2534, 2536, 2538, 2543, 2545, 2549, 2553, 2555, 2560, 2563, 2566, 2568, 2570, 2577, 2579, 2582, 2586, 2591, 2596, 2598, 2600, 2602, 2605, 2607, 2610, 2613, 2618, 2620, 2622, 2625, 2630, 2633, 2635, 2637, 2639, 2643, 2645, 2651, 2653, 2655, 2657, 2660, 2662, 2664, 2666, 2669, 2671, 2673, 2676, 2678, 2680, 2682, 2684, 2686, 2688, 2690, 2692, 2694, 2696, 2698, 2700, 2702, 2705, 2707, 2709, 2713, 2715, 2718, 2720, 2723, 2725, 2727, 2729, 2731, 2733, 2737, 2742, 2744, 2747, 2749, 2752, 2755, 2757, 2759, 2761, 2763, 2765, 2767, 2769, 2774, 2776, 2779, 2783, 2785, 2787, 2789, 2793, 2795, 2798, 2800, 2805, 2808, 2812, 2814, 2817, 2820, 2824, 2827, 2829, 2831, 2834, 2837, 2839, 2841, 2843, 2848, 2854, 2860, 2865, 2871, 2874, 2879, 2881, 2884, 2887, 2889, 2895, 2897, 2899, 2901, 2905, 2912, 2914, 2916, or 2925, or a fragment thereof. A plant produced from the plant cell has a difference in the level of biomass as compared to the corresponding level of biomass of a control plant that does not comprise the exogenous nucleic acid. A transgenic plant comprising such a plant cell is also provided. Also provided is a plant biomass or seed product. The product comprises vegetative or embryonic tissue from a transgenic plant described herein.

Isolated nucleic acids are also provided. In one aspect, an isolated nucleic acid comprises a nucleotide sequence having 80% or greater sequence identity to the nucleotide sequence set forth in SEQ ID NO:1, 3, 7, 9, 11, 14, 16, 19, 21, 23, 28, 31, 33, 36, 40, 42, 44, 46, 48, 50, 52, 54, 58, 61, 66, 68, 72, 76, 78, 87, 89, 114, 116, 118, 125, 127, 130, 132, 134, 136, 138, 143, 145, 151, 153, 156, 159, 161, 164, 166, 168, 170, 172, 174, 177, 179, 181, 184, 186, 188, 190, 192, 202, 204, 206, 210, 212, 216, 221, 225, 227, 230, 241, 243, 253, 280, 283, 285, 290, 292, 296, 298, 301, 303, 305, 309, 312, 314, 321, 323, 325, 327, 329, 332, 334, 336, 339, 341, 344, 348, 352, 356, 359, 361, 366, 368, 376, 381, 388, 392, 394, 397, 399, 402, 405, 407, 413, 416, 418, 455, 458, 460, 463, 465, 471, 473, 476, 478, 481, 486, 496, 498, 500, 505, 508, 513, 519, 521, 523, 525, 527, 529, 541, 544, 549, 552, 556, 558, 561, 564, 567, 569, 571, 574, 578, 588, 594, 600, 603, 606, 608, 628, 630, 632, 639, 641, 643, 645, 647, 649, 652, 661, 668, 670, 673, 675, 678, 681, 683, 686, 688, 692, 695, 699, 701, 703, 708, 711, 713, 716, 724, 729, 736, 738, 740, 742, 748, 750, 754, 756, 758, 761, 763, 765, 767, 769, 771, 774, 777, 779, 781, 783, 785, 787, 790, 792, 797, 799, 803, 806, 808, 810, 812, 814, 816, 818, 821, 826, 829, 831, 833, 835, 837, 839, 841, 845, 847, 852, 854, 859, 862, 865, 867, 869, 872, 875, 877, 879, 881, 884, 886, 888, 891, 894, 898, 900, 904, 906, 911, 913, 918, 920, 922, 924, 927, 929, 931, 933, 938, 940, 942, 944, 946, 948, 951, 954, 956, 958, 961, 963, 966, 969, 971, 973, 975, 977, 979, 982, 984, 988, 990, 992, 994, 996, 999, 1001, 1004, 1006, 1010, 1012, 1015, 1017, 1019, 1021, 1028, 1030, 1032, 1035, 1037, 1040, 1047, 1053, 1055, 1057, 1059, 1063, 1065, 1068, 1073, 1075, 1080, 1082, 1084, 1086, 1089, 1091, 1094, 1098, 1100, 1103, 1106, 1109, 1116, 1118, 1123, 1125, 1127, 1129, 1131, 1134, 1136, 1138, 1140, 1143, 1145, 1147, 1149, 1151, 1153, 1155, 1159, 1161, 1163, 1165, 1167, 1170, 1172, 1174, 1178, 1182, 1184, 1186, 1189, 1191, 1195, 1197, 1199, 1202, 1204, 1206, 1208, 1211, 1213, 1215, 1217, 1220, 1223, 1225, 1227, 1229, 1231, 1233, 1237, 1239, 1242, 1244, 1246, 1248, 1250, 1254, 1256, 1258, 1260, 1262, 1264, 1267, 1269, 1271, 1273, 1275, 1277, 1280, 1282, 1284, 1286, 1288, 1290, 1292, 1295, 1298, 1301, 1303, 1305, 1307, 1311, 1315, 1317, 1319, 1321, 1323, 1327, 1331, 1335, 1337, 1346, 1348, 1351, 1354, 1356, 1362, 1365, 1368, 1374, 1378, 1383, 1385, 1387, 1390, 1392, 1394, 1396, 1398, 1400, 1404, 1406, 1408, 1411, 1413, 1415, 1417, 1419, 1423, 1425, 1427, 1430, 1432, 1434, 1436, 1438, 1442, 1444, 1447, 1449, 1451, 1453, 1455, 1458, 1460, 1462, 1464, 1466, 1468, 1470, 1472, 1474, 1476, 1478, 1480, 1484, 1487, 1491, 1494, 1496, 1499, 1501, 1504, 1510, 1513, 1517, 1519, 1521, 1527, 1530, 1532, 1536, 1539, 1541, 1543, 1545, 1548, 1551, 1553, 1556, 1559, 1561, 1566, 1568, 1570, 1573, 1576, 1579, 1582, 1585, 1587, 1590, 1592, 1597, 1601, 1606, 1608, 1610, 1614, 1617, 1620, 1623, 1625, 1627, 1631, 1633, 1635, 1638, 1641, 1643, 1645, 1651, 1653, 1655, 1659, 1663, 1665, 1667, 1673, 1675, 1678, 1685, 1687, 1690, 1692, 1694, 1696, 1698, 1703, 1709, 1716, 1718, 1720, 1722, 1726, 1728, 1730, 1733, 1736, 1738, 1740, 1742, 1744, 1748, 1750, 1752, 1754, 1756, 1760, 1763, 1768, 1771, 1775, 1778, 1780, 1782, 1785, 1787, 1789, 1791, 1793, 1796, 1800, 1802, 1814, 1818, 1820, 1824, 1830, 1832, 1836, 1838, 1840, 1843, 1846, 1848, 1851, 1860, 1862, 1864, 1868, 1870, 1873, 1875, 1878, 1880, 1884, 1886, 1888, 1890, 1892, 1896, 1899, 1901, 1905, 1907, 1909, 1911, 1914, 1916, 1921, 1923, 1925, 1927, 1930, 1932, 1934, 1937, 1939, 1941, 1943, 1945, 1947, 1950, 1952, 1954, 1956, 1958, 1960, 1962, 1964, 1966, 1968, 1970, 1977, 1981, 1984, 1988, 1991, 1993, 1995, 1998, 2001, 2004, 2006, 2012, 2014, 2020, 2025, 2027, 2029, 2033, 2035, 2039, 2045, 2050, 2052, 2054, 2057, 2059, 2061, 2063, 2065, 2068, 2071, 2080, 2082, 2084, 2086, 2089, 2094, 2096, 2099, 2101, 2103, 2107, 2109, 2111, 2114, 2116, 2123, 2125, 2127, 2131, 2134, 2139, 2145, 2147, 2155, 2157, 2164, 2166, 2168, 2180, 2182, 2185, 2189, 2191, 2193, 2204, 2206, 2208, 2210, 2212, 2214, 2216, 2218, 2220, 2222, 2224, 2227, 2230, 2232, 2234, 2242, 2245, 2247, 2249, 2252, 2254, 2256, 2258, 2260, 2263, 2266, 2268, 2271, 2274, 2277, 2280, 2283, 2287, 2290, 2292, 2294, 2301, 2303, 2305, 2308, 2311, 2319, 2322, 2324, 2326, 2328, 2331, 2333, 2336, 2338, 2340, 2342, 2344, 2349, 2351, 2353, 2355, 2359, 2363, 2365, 2368, 2370, 2372, 2374, 2377, 2379, 2382, 2385, 2387, 2389, 2393, 2395, 2398, 2400, 2404, 2406, 2410, 2412, 2414, 2416, 2420, 2426, 2431, 2433, 2436, 2438, 2444, 2447, 2450, 2453, 2455, 2465, 2467, 2473, 2479, 2481, 2492, 2501, 2504, 2508, 2515, 2520, 2524, 2526, 2528, 2532, 2534, 2536, 2538, 2543, 2545, 2549, 2553, 2555, 2560, 2563, 2566, 2568, 2570, 2577, 2579, 2582, 2586, 2591, 2596, 2598, 2600, 2602, 2605, 2607, 2610, 2613, 2618, 2620, 2622, 2625, 2630, 2633, 2635, 2637, 2639, 2643, 2645, 2651, 2653, 2655, 2657, 2660, 2662, 2664, 2666, 2669, 2671, 2673, 2676, 2678, 2680, 2682, 2684, 2686, 2688, 2690, 2692, 2694, 2696, 2698, 2700, 2702, 2705, 2707, 2709, 2713, 2715, 2718, 2720, 2723, 2725, 2727, 2729, 2731, 2733, 2737, 2742, 2744, 2747, 2749, 2752, 2755, 2757, 2759, 2761, 2763, 2765, 2767, 2769, 2774, 2776, 2779, 2783, 2785, 2787, 2789, 2793, 2795, 2798, 2800, 2805, 2808, 2812, 2814, 2817, 2820, 2824, 2827, 2829, 2831, 2834, 2837, 2839, 2841, 2843, 2848, 2854, 2860, 2865, 2871, 2874, 2879, 2881, 2884, 2887, 2889, 2895, 2897, 2899, 2901, 2905, 2912, 2914, 2916, or 2925. In another aspect, an isolated nucleic acid comprises a nucleotide sequence encoding a polypeptide having 80% or greater sequence identity to the amino acid sequence set forth in SEQ ID NO:2, 4, 5, 6, 8, 10, 12, 13, 15, 17, 18, 20, 22, 24, 25, 26, 27, 29, 30, 32, 34, 35, 37, 38, 39, 41, 43, 45, 47, 49, 51, 53, 55, 56, 57, 59, 60, 62, 63, 64, 65, 67, 69, 70, 71, 73, 74, 75, 77, 79, 80, 81, 82, 83, 84, 85, 86, 88, 90, 91, 92, 93, 94, 95, 96, 97, 98, 99, 100, 101, 102, 103, 104, 105, 106, 107, 108, 109, 110, 111, 112, 113, 115, 117, 119, 120, 121, 122, 123, 124, 126, 128, 129, 131, 133, 135, 137, 139, 140, 141, 142, 144, 146, 147, 148, 149, 150, 152, 154, 155, 157, 158, 160, 162, 163, 165, 167, 169, 171, 173, 175, 176, 178, 180, 182, 183, 185, 187, 189, 191, 193, 194, 195, 196, 197, 198, 199, 200, 201, 203, 205, 207, 208, 209, 211, 213, 214, 215, 217, 218, 219, 220, 222, 223, 224, 226, 228, 229, 231, 232, 233, 234, 235, 236, 237, 238, 239, 240, 242, 244, 245, 246, 247, 248, 249, 250, 251, 252, 254, 255, 256, 257, 258, 259, 260, 261, 262, 263, 264, 265, 266, 267, 268, 269, 270, 271, 272, 273, 274, 275, 276, 277, 278, 279, 281, 282, 284, 286, 287, 288, 289, 291, 293, 294, 295, 297, 299, 300, 302, 304, 306, 307, 308, 310, 311, 313, 315, 316, 317, 318, 319, 320, 322, 324, 326, 328, 330, 331, 333, 335, 337, 338, 340, 342, 343, 345, 346, 347, 349, 350, 351, 353, 354, 355, 357, 358, 360, 362, 363, 364, 365, 367, 369, 370, 371, 372, 373, 374, 375, 377, 378, 379, 380, 382, 383, 384, 385, 386, 387, 389, 390, 391, 393, 395, 396, 398, 400, 401, 403, 404, 406, 408, 409, 410, 411, 412, 414, 415, 417, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 456, 457, 459, 461, 462, 464, 466, 467, 468, 469, 470, 472, 474, 475, 477, 479, 480, 482, 483, 484, 485, 487, 488, 489, 490, 491, 492, 493, 494, 495, 497, 499, 501, 502, 503, 504, 506, 507, 509, 510, 511, 512, 514, 515, 516, 517, 518, 520, 522, 524, 526, 528, 530, 531, 532, 533, 534, 535, 536, 537, 538, 539, 540, 542, 543, 545, 546, 547, 548, 550, 551, 553, 554, 555, 557, 559, 560, 562, 563, 565, 566, 568, 570, 572, 573, 575, 576, 577, 579, 580, 581, 582, 583, 584, 585, 586, 587, 589, 590, 591, 592, 593, 595, 596, 597, 598, 599, 601, 602, 604, 605, 607, 609, 610, 611, 612, 613, 614, 615, 616, 617, 618, 619, 620, 621, 622, 623, 624, 625, 626, 627, 629, 631, 633, 634, 635, 36, 637, 638, 640, 642, 644, 646, 648, 650, 651, 653, 654, 655, 656, 657, 658, 659, 660, 662, 663, 664, 665, 666, 667, 669, 671, 672, 674, 676, 677, 679, 680, 682, 684, 685, 687, 689, 690, 691, 693, 694, 696, 697, 698, 00, 702, 704, 705, 706, 707, 709, 710, 712, 714, 715, 717, 718, 719, 720, 721, 722, 723, 725, 726, 727, 728, 730, 731, 732, 733, 734, 735, 737, 739, 741, 743, 744, 745, 746, 747, 749, 751, 752, 753, 755, 757, 759, 760, 762, 764, 766, 768, 770, 772, 773, 775, 776, 778, 780, 782, 784, 786, 788, 789, 791, 793, 794, 795, 796, 798, 800, 801, 802, 804, 805, 807, 809, 811, 813, 815, 817, 819, 820, 822, 823, 824, 825, 827, 828, 830, 832, 834, 836, 838, 840, 842, 843, 844, 846, 848, 849, 850, 851, 853, 855, 856, 857, 858, 860, 861, 863, 864, 866, 868, 870, 871, 873, 874, 876, 878, 880, 882, 883, 885, 887, 889, 890, 892, 893, 895, 896, 897, 899, 901, 902, 903, 905, 907, 908, 909, 910, 912, 914, 915, 916, 917, 919, 921, 923, 925, 926, 928, 930, 932, 934, 935, 936, 937, 939, 941, 943, 945, 947, 949, 950, 952, 953, 955, 957, 959, 960, 962, 964, 965, 967, 968, 970, 972, 974, 976, 978, 980, 981, 983, 985, 86, 87, 989, 991, 993, 995, 997, 998, 1000, 1002, 1003, 1005, 1007, 1008, 1009, 1011, 1013, 1014, 1016, 1018, 1020, 1022, 1023, 1024, 1025, 1026, 1027, 1029, 1031, 1033, 1034, 1036, 1038, 1039, 1041, 1042, 1043, 1044, 1045, 1046, 1048, 1049, 1050, 1051, 1052, 1054, 1056, 1058, 1060, 1061, 1062, 1064, 1066, 1067, 1069, 1070, 1071, 1072, 1074, 1076, 1077, 1078, 1079, 1081, 1083, 1085, 1087, 1088, 1090, 1092, 1093, 1095, 1096, 1097, 1099, 1101, 1102, 1104, 1105, 1107, 1108, 1110, 1111, 1112, 1113, 1114, 1115, 1117, 1119, 1120, 1121, 1122, 1124, 1126, 1128, 1130, 1132, 1133, 1135, 1137, 1139, 1141, 1142, 1144, 1146, 1148, 1150, 1152, 1154, 1156, 1157, 1158, 1160, 1162, 1164, 1166, 1168, 1169, 1171, 1173, 1175, 1176, 1177, 1179, 1180, 1181, 1183, 1185, 1187, 1188, 1190, 1192, 1193, 1194, 1196, 1198, 1200, 1201, 1203, 1205, 1207, 1209, 1210, 1212, 1214, 1216, 1218, 1219, 1221, 1222, 1224, 1226, 1228, 1230, 1232, 1234, 1235, 1236, 1238, 1240, 1241, 1243, 1245, 1247, 1249, 1251, 1252, 1253, 1255, 1257, 1259, 1261, 1263, 1265, 1266, 1268, 1270, 1272, 1274, 1276, 1278, 1279, 1281, 1283, 1285, 1287, 1289, 1291, 1293, 1294, 1296, 1297, 1299, 1300, 1302, 1304, 1306, 1308, 1309, 1310, 1312, 1313, 1314, 1316, 1318, 1320, 1322, 1324, 1325, 1326, 1328, 1329, 1330, 1332, 1333, 1334, 1336, 1338, 1339, 1340, 1341, 1342, 1343, 1344, 1345, 1347, 1349, 1350, 1352, 1353, 1355, 1357, 1358, 1359, 1360, 1361, 1363, 1364, 1366, 1367, 1369, 1370, 1371, 1372, 1373, 1375, 1376, 1377, 1379, 1380, 1381, 1382, 1384, 1386, 1388, 1389, 1391, 1393, 1395, 1397, 1399, 1401, 1402, 1403, 1405, 1407, 1409, 1410, 1412, 1414, 1416, 1418, 1420, 1421, 1422, 1424, 1426, 1428, 1429, 1431, 1433, 1435, 1437, 1439, 1440, 1441, 1443, 1445, 1446, 1448, 1450, 1452, 1454, 1456, 1457, 1459, 1461, 1463, 1465, 1467, 1469, 1471, 1473, 1475, 1477, 1479, 1481, 1482, 1483, 1485, 1486, 1488, 1489, 1490, 1492, 1493, 1495, 1497, 1498, 1500, 1502, 1503, 1505, 1506, 1507, 1508, 1509, 1511, 1512, 1514, 1515, 1516, 1518, 1520, 1522, 1523, 1524, 1525, 1526, 1528, 1529, 1531, 1533, 1534, 1535, 1537, 1538, 1540, 1542, 1544, 1546, 1547, 1549, 1550, 1552, 1554, 1555, 1557, 1558, 1560, 1562, 1563, 1564, 1565, 1568, 1569, 1571, 1572, 1574, 1575, 1577, 1578, 1580, 1581, 1583, 1584, 1586, 1588, 1589, 1591, 1593, 1594, 1595, 1596, 1598, 1599, 1600, 1602, 1603, 1604, 1605, 1607, 1609, 1611, 1612, 1613, 1615, 1616, 1618, 1619, 1621, 1622, 1624, 1626, 1628, 1629, 1630, 1632, 1634, 1636, 1637, 1639, 1640, 1642, 1644, 1646, 1647, 1648, 1649, 1650, 1652, 1654, 1656, 1657, 1658, 1660, 1661, 1662, 1664, 1666, 1668, 1669, 1670, 1671, 1672, 1674, 1676, 1677, 1679, 1680, 1681, 1682, 1683, 1684, 1686, 1688, 1689, 1691, 1693, 1695, 1697, 1699, 1700, 1701, 1702, 1704, 1705, 1706, 1707, 1708, 1710, 1711, 1712, 1713, 1714, 1715, 1717, 1719, 1721, 1723, 1724, 1725, 1727, 1729, 1731, 1732, 1734, 1735, 1737, 1739, 1741, 1743, 1745, 1746, 1747, 1749, 1751, 1753, 1755, 1757, 1758, 1759, 1761, 1762, 1764, 1765, 1766, 1767, 1769, 1770, 1772, 1773, 1774, 1776, 1777, 1779, 1781, 1783, 1784, 1786, 1788, 1790, 1792, 1794, 1795, 1797, 798, 1799, 1801, 1803, 1804, 1805, 1806, 1807, 1808, 1809, 1810, 1811, 1812, 1813, 1815, 1816, 1817, 1819, 1821, 1822, 1823, 1825, 1826, 1827, 1828, 1829, 1831, 1833, 1834, 1835, 1837, 1839, 1841, 1842, 1844, 1845, 1847, 1849, 1850, 1852, 1853, 1854, 1855, 1856, 1857, 1858, 1859, 1861, 1863, 1865, 1866, 1867, 1869, 1871, 1872, 1874, 1876, 1877, 1879, 1881, 1882, 1883, 1885, 1887, 1889, 1891, 1893, 1894, 1895, 1897, 1898, 1900, 1902, 1903, 1904, 1906, 1908, 1910, 1912, 1913, 1915, 1917, 1918, 1919, 1920, 1922, 1924, 1926, 1928, 1929, 1931, 1933, 1935, 1936, 1938, 1940, 1942, 1944, 1946, 1948, 1949, 1951, 1953, 1955, 1957, 1959, 1961, 1963, 1965, 1967, 1969, 1971, 1972, 1973, 1974, 1975, 1976, 1978, 1979, 1980, 1982, 1983, 1985, 1986, 1987, 1989, 1990, 1992, 1994, 1996, 1997, 1999, 2000, 2002, 2003, 2005, 2007, 2008, 2009, 2010, 2011, 2013, 2015, 2016, 2017, 2018, 2019, 2021, 2022, 2023, 2024, 2026, 2028, 2030, 2031, 2032, 2034, 2036, 2037, 2038, 2040, 2041, 2042, 2043, 2044, 2046, 2047, 2048, 2049, 2051, 2053, 2055, 2056, 2058, 2060, 2062, 2064, 2066, 2067, 2069, 2070, 2072, 2073, 2074, 2075, 2076, 2077, 2078, 2079, 2081, 2083, 2085, 2087, 2088, 2090, 2091, 2092, 2093, 2095, 2097, 2098, 2100, 2102, 2104, 2105, 2106, 2108, 2110, 2112, 2113, 2115, 2117, 2118, 2119, 2120, 2121, 2122, 2124, 2126, 2128, 2129, 2130, 2132, 2133, 2135, 2136, 2137, 2138, 2140, 2141, 2142, 2143, 2144, 2146, 2148, 2149, 2150, 2151, 2152, 2153, 2154, 2156, 2158, 2159, 2160, 2161, 2162, 2163, 2165, 2167, 2169, 2170, 2171, 2172, 2173, 2174, 2175, 2176, 2177, 2178, 2179, 2181, 2183, 2184, 2186, 2187, 2188, 2190, 2192, 2194, 2195, 2196, 2197, 2198, 2199, 2200, 2201, 2202, 2203, 2205, 2207, 2209, 2211, 2213, 2215, 2217, 2219, 2221, 2223, 2225, 2226, 2228, 2229, 2231, 2233, 2235, 2236, 2237, 2238, 2239, 2240, 2241, 2243, 2244, 2246, 2248, 2250, 2251, 2253, 2255, 2257, 2259, 2261, 2262, 2264, 2265, 2267, 2269, 2270, 2272, 2273, 2275, 2276, 2278, 2279, 2281, 2282, 2284, 2285, 2286, 2288, 2289, 2291, 2293, 2295, 2296, 2297, 2298, 2299, 2300, 2302, 2304, 2306, 2307, 2309, 2310, 2312, 2313, 2314, 2315, 2316, 2317, 2318, 2320, 2321, 2323, 2325, 2327, 2329, 2330, 2332, 2334, 2335, 2337, 2339, 2341, 2343, 2345, 2346, 2347, 2348, 2350, 2352, 2354, 2356, 2357, 2358, 2360, 2361, 2362, 2364, 2366, 2367, 2369, 2371, 2373, 2375, 2376, 2378, 2380, 2381, 2383, 2384, 2386, 2388, 2390, 2391, 2392, 2394, 2396, 2397, 2399, 2401, 2402, 2403, 2405, 2407, 2408, 2409, 2411, 2413, 2415, 2417, 2418, 2419, 2421, 2422, 2423, 2424, 2425, 2427, 2428, 2429, 2430, 2432, 2434, 2435, 2437, 2439, 2440, 2441, 2442, 2443, 2445, 2446, 2448, 2449, 2451, 2452, 2454, 2456, 2457, 2458, 2459, 2460, 2461, 2462, 2463, 2464, 2466, 2468, 2469, 2470, 2471, 2472, 2474, 2475, 2476, 2477, 2478, 2480, 2482, 2483, 2484, 2485, 2486, 2487, 2488, 2489, 2490, 2491, 2493, 2494, 2495, 2496, 2497, 2498, 2499, 2500, 2502, 2503, 2505, 2506, 2507, 2509, 2510, 2511, 2512, 2513, 2514, 2516, 2517, 2518, 2519, 2521, 2522, 2523, 2525, 2527, 2529, 2530, 2531, 2533, 2535, 2537, 2539, 2540, 2541, 2542, 2544, 2546, 2547, 2548, 2550, 2551, 2552, 2554, 2556, 2557, 2558, 2559, 2561, 2562, 2564, 2565, 2567, 2569, 2571, 2572, 2573, 2574, 2575, 2576, 2578, 2580, 2581, 2583, 2584, 2585, 2587, 2588, 2589, 2590, 2592, 2593, 2594, 2595, 2597, 2599, 2601, 2603, 2604, 2606, 2608, 2609, 2611, 2612, 2614, 2615, 2616, 2617, 2619, 2621, 2623, 2624, 2626, 2627, 2628, 2629, 2631, 2632, 2634, 2636, 2638, 2640, 2641, 2642, 2644, 2646, 2647, 2648, 2649, 2650, 2652, 2654, 2656, 2658, 2659, 2661, 2663, 2665, 2667, 2668, 2670, 2672, 2674, 2675, 2677, 2679, 2681, 2683, 2685, 2687, 2689, 2691, 2693, 2695, 2697, 2699, 2701, 2703, 2704, 2706, 2708, 2710, 2711, 2712, 2714, 2716, 2717, 2719, 2721, 2722, 2724, 2726, 2728, 2730, 2732, 2734, 2735, 2736, 2738, 2739, 2740, 2741, 2743, 2745, 2746, 2748, 2750, 2751, 2753, 2754, 2756, 2758, 2760, 2762, 2764, 2766, 2768, 2770, 2771, 2772, 2773, 2775, 2777, 2778, 2780, 2781, 2782, 2784, 2786, 2788, 2790, 2791, 2792, 2794, 2796, 2797, 2799, 2801, 2802, 2803, 2804, 2806, 2807, 2809, 2810, 2811, 2813, 2815, 2816, 2818, 2819, 2821, 2822, 2823, 2825, 2826, 2828, 2830, 2832, 2833, 2835, 2836, 2838, 2840, 2842, 2844, 2845, 2846, 2847, 2849, 2850, 2851, 2852, 2853, 2855, 2856, 2857, 2858, 2859, 2861, 2862, 2863, 2864, 2866, 2867, 2868, 2869, 2870, 2872, 2873, 2875, 2876, 2877, 2878, 2880, 2882, 2883, 2885, 2886, 2888, 2890, 2891, 2892, 2893, 2894, 2896, 2898, 2900, 2902, 2903, 2904, 2906, 2907, 2908, 2909, 2910, 2911, 2915, 2917, 2918, 2919, 2920, 2921, 2922, 2923, 2924, or 2926.

In another aspect, methods of identifying a genetic polymorphism associated with variation in the level of biomass are provided. The methods include providing a population of plants, and determining whether one or more genetic polymorphisms in the population are genetically linked to the locus for a polypeptide selected from the group consisting of the polypeptides depicted in FIGS. 1-50 and functional homologs thereof. The correlation between variation in the level of biomass in a tissue in plants of the population and the presence of the one or more genetic polymorphisms in plants of the population is measured, thereby permitting identification of whether or not the one or more genetic polymorphisms are associated with such variation.

In another aspect, methods of making a plant line are provided. The methods include determining whether one or more genetic polymorphisms in a population of plants is associated with the locus for one or more of the polypeptides depicted in FIGS. 1-50 and functional homologs of such polypeptides. One or more plants in the population is identified in which the presence of at least one of the genetic polymorphism(s) is associated with variation in a biomass trait. The above-described steps can be performed in either order. One or more of the identified plants is then crossed with itself or a different plant to produce seed, and at least one progeny plant grown from such seed is crossed with itself or a different plant. The steps of selfing and outcrossing are repeated for an additional 0-5 generations to make a plant line in which the at least one polymorphism is present. The biomass trait can be yield of dry matter, and the plant population can be switchgrass plants.

This document also features a method of altering the level of biomass in a plant, where the method includes modifying an endogenous biomass-modulating nucleic acid. The nucleic acid comprises a nucleotide sequence with an open reading frame having 80 percent or greater sequence identity (e.g., 90% or greater, or 95% or greater) to the nucleotide sequence selected from the group consisting of SEQ ID NO: 1, 3, 7, 9, 11, 14, 16, 19, 21, 23, 28, 31, 33, 36, 40, 42, 44, 46, 48, 50, 52, 54, 58, 61, 66, 68, 72, 76, 78, 87, 89, 114, 116, 118, 125, 127, 130, 132, 134, 136, 138, 143, 145, 151, 153, 156, 159, 161, 164, 166, 168, 170, 172, 174, 177, 179, 181, 184, 186, 188, 190, 192, 202, 204, 206, 210, 212, 216, 221, 225, 227, 230, 241, 243, 253, 280, 283, 285, 290, 292, 296, 298, 301, 303, 305, 309, 312, 314, 321, 323, 325, 327, 329, 332, 334, 336, 339, 341, 344, 348, 352, 356, 359, 361, 366, 368, 376, 381, 388, 392, 394, 397, 399, 402, 405, 407, 413, 416, 418, 455, 458, 460, 463, 465, 471, 473, 476, 478, 481, 486, 496, 498, 500, 505, 508, 513, 519, 521, 523, 525, 527, 529, 541, 544, 549, 552, 556, 558, 561, 564, 567, 569, 571, 574, 578, 588, 594, 600, 603, 606, 608, 628, 630, 632, 639, 641, 643, 645, 647, 649, 652, 661, 668, 670, 673, 675, 678, 681, 683, 686, 688, 692, 695, 699, 701, 703, 708, 711, 713, 716, 724, 729, 736, 738, 740, 742, 748, 750, 754, 756, 758, 761, 763, 765, 767, 769, 771, 774, 777, 779, 781, 783, 785, 787, 790, 792, 797, 799, 803, 806, 808, 810, 812, 814, 816, 818, 821, 826, 829, 831, 833, 835, 837, 839, 841, 845, 847, 852, 854, 859, 862, 865, 867, 869, 872, 875, 877, 879, 881, 884, 886, 888, 891, 894, 898, 900, 904, 906, 911, 913, 918, 920, 922, 924, 927, 929, 931, 933, 938, 940, 942, 944, 946, 948, 951, 954, 956, 958, 961, 963, 966, 969, 971, 973, 975, 977, 979, 982, 984, 988, 990, 992, 994, 996, 999, 1001, 1004, 1006, 1010, 1012, 1015, 1017, 1019, 1021, 1028, 1030, 1032, 1035, 1037, 1040, 1047, 1053, 1055, 1057, 1059, 1063, 1065, 1068, 1073, 1075, 1080, 1082, 1084, 1086, 1089, 1091, 1094, 1098, 1100, 1103, 1106, 1109, 1116, 1118, 1123, 1125, 1127, 1129, 1131, 1134, 1136, 1138, 1140, 1143, 1145, 1147, 1149, 1151, 1153, 1155, 1159, 1161, 1163, 1165, 1167, 1170, 1172, 1174, 1178, 1182, 1184, 1186, 1189, 1191, 1195, 1197, 1199, 1202, 1204, 1206, 1208, 1211, 1213, 1215, 1217, 1220, 1223, 1225, 1227, 1229, 1231, 1233, 1237, 1239, 1242, 1244, 1246, 1248, 1250, 1254, 1256, 1258, 1260, 1262, 1264, 1267, 1269, 1271, 1273, 1275, 1277, 1280, 1282, 1284, 1286, 1288, 1290, 1292, 1295, 1298, 1301, 1303, 1305, 1307, 1311, 1315, 1317, 1319, 1321, 1323, 1327, 1331, 1335, 1337, 1346, 1348, 1351, 1354, 1356, 1362, 1365, 1368, 1374, 1378, 1383, 1385, 1387, 1390, 1392, 1394, 1396, 1398, 1400, 1404, 1406, 1408, 1411, 1413, 1415, 1417, 1419, 1423, 1425, 1427, 1430, 1432, 1434, 1436, 1438, 1442, 1444, 1447, 1449, 1451, 1453, 1455, 1458, 1460, 1462, 1464, 1466, 1468, 1470, 1472, 1474, 1476, 1478, 1480, 1484, 1487, 1491, 1494, 1496, 1499, 1501, 1504, 1510, 1513, 1517, 1519, 1521, 1527, 1530, 1532, 1536, 1539, 1541, 1543, 1545, 1548, 1551, 1553, 1556, 1559, 1561, 1566, 1568, 1570, 1573, 1576, 1579, 1582, 1585, 1587, 1590, 1592, 1597, 1601, 1606, 1608, 1610, 1614, 1617, 1620, 1623, 1625, 1627, 1631, 1633, 1635, 1638, 1641, 1643, 1645, 1651, 1653, 1655, 1659, 1663, 1665, 1667, 1673, 1675, 1678, 1685, 1687, 1690, 1692, 1694, 1696, 1698, 1703, 1709, 1716, 1718, 1720, 1722, 1726, 1728, 1730, 1733, 1736, 1738, 1740, 1742, 1744, 1748, 1750, 1752, 1754, 1756, 1760, 1763, 1768, 1771, 1775, 1778, 1780, 1782, 1785, 1787, 1789, 1791, 1793, 1796, 1800, 1802, 1814, 1818, 1820, 1824, 1830, 1832, 1836, 1838, 1840, 1843, 1846, 1848, 1851, 1860, 1862, 1864, 1868, 1870, 1873, 1875, 1878, 1880, 1884, 1886, 1888, 1890, 1892, 1896, 1899, 1901, 1905, 1907, 1909, 1911, 1914, 1916, 1921, 1923, 1925, 1927, 1930, 1932, 1934, 1937, 1939, 1941, 1943, 1945, 1947, 1950, 1952, 1954, 1956, 1958, 1960, 1962, 1964, 1966, 1968, 1970, 1977, 1981, 1984, 1988, 1991, 1993, 1995, 1998, 2001, 2004, 2006, 2012, 2014, 2020, 2025, 2027, 2029, 2033, 2035, 2039, 2045, 2050, 2052, 2054, 2057, 2059, 2061, 2063, 2065, 2068, 2071, 2080, 2082, 2084, 2086, 2089, 2094, 2096, 2099, 2101, 2103, 2107, 2109, 2111, 2114, 2116, 2123, 2125, 2127, 2131, 2134, 2139, 2145, 2147, 2155, 2157, 2164, 2166, 2168, 2180, 2182, 2185, 2189, 2191, 2193, 2204, 2206, 2208, 2210, 2212, 2214, 2216, 2218, 2220, 2222, 2224, 2227, 2230, 2232, 2234, 2242, 2245, 2247, 2249, 2252, 2254, 2256, 2258, 2260, 2263, 2266, 2268, 2271, 2274, 2277, 2280, 2283, 2287, 2290, 2292, 2294, 2301, 2303, 2305, 2308, 2311, 2319, 2322, 2324, 2326, 2328, 2331, 2333, 2336, 2338, 2340, 2342, 2344, 2349, 2351, 2353, 2355, 2359, 2363, 2365, 2368, 2370, 2372, 2374, 2377, 2379, 2382, 2385, 2387, 2389, 2393, 2395, 2398, 2400, 2404, 2406, 2410, 2412, 2414, 2416, 2420, 2426, 2431, 2433, 2436, 2438, 2444, 2447, 2450, 2453, 2455, 2465, 2467, 2473, 2479, 2481, 2492, 2501, 2504, 2508, 2515, 2520, 2524, 2526, 2528, 2532, 2534, 2536, 2538, 2543, 2545, 2549, 2553, 2555, 2560, 2563, 2566, 2568, 2570, 2577, 2579, 2582, 2586, 2591, 2596, 2598, 2600, 2602, 2605, 2607, 2610, 2613, 2618, 2620, 2622, 2625, 2630, 2633, 2635, 2637, 2639, 2643, 2645, 2651, 2653, 2655, 2657, 2660, 2662, 2664, 2666, 2669, 2671, 2673, 2676, 2678, 2680, 2682, 2684, 2686, 2688, 2690, 2692, 2694, 2696, 2698, 2700, 2702, 2705, 2707, 2709, 2713, 2715, 2718, 2720, 2723, 2725, 2727, 2729, 2731, 2733, 2737, 2742, 2744, 2747, 2749, 2752, 2755, 2757, 2759, 2761, 2763, 2765, 2767, 2769, 2774, 2776, 2779, 2783, 2785, 2787, 2789, 2793, 2795, 2798, 2800, 2805, 2808, 2812, 2814, 2817, 2820, 2824, 2827, 2829, 2831, 2834, 2837, 2839, 2841, 2843, 2848, 2854, 2860, 2865, 2871, 2874, 2879, 2881, 2884, 2887, 2889, 2895, 2897, 2899, 2901, 2905, 2912, 2914, 2916, and 2925, wherein said plant has a difference in the level of biomass as compared to the corresponding level of a control plant where said nucleic acid has not been modified. The modification can be effected by introducing a genetic modification in the locus comprising the nucleic acid. The method further can include selecting for plants having altered biomass. In some embodiments, the endogenous nucleic acid encodes a polypeptide having 80 percent or greater sequence identity to an amino acid sequence selected from the group consisting of SEQ ID NO: 2, 4, 5, 6, 8, 10, 12, 13, 15, 17, 18, 20, 22, 24, 25, 26, 27, 29, 30, 32, 34, 35, 37, 38, 39, 41, 43, 45, 47, 49, 51, 53, 55, 56, 57, 59, 60, 62, 63, 64, 65, 67, 69, 70, 71, 73, 74, 75, 77, 79, 80, 81, 82, 83, 84, 85, 86, 88, 90, 91, 92, 93, 94, 95, 96, 97, 98, 99, 100, 101, 102, 103, 104, 105, 106, 107, 108, 109, 110, 111, 112, 113, 115, 117, 119, 120, 121, 122, 123, 124, 126, 128, 129, 131, 133, 135, 137, 139, 140, 141, 142, 144, 146, 147, 148, 149, 150, 152, 154, 155, 157, 158, 160, 162, 163, 165, 167, 169, 171, 173, 175, 176, 178, 180, 182, 183, 185, 187, 189, 191, 193, 194, 195, 196, 197, 198, 199, 200, 201, 203, 205, 207, 208, 209, 211, 213, 214, 215, 217, 218, 219, 220, 222, 223, 224, 226, 228, 229, 231, 232, 233, 234, 235, 236, 237, 238, 239, 240, 242, 244, 245, 246, 247, 248, 249, 250, 251, 252, 254, 255, 256, 257, 258, 259, 260, 261, 262, 263, 264, 265, 266, 267, 268, 269, 270, 271, 272, 273, 274, 275, 276, 277, 278, 279, 281, 282, 284, 286, 287, 288, 289, 291, 293, 294, 295, 297, 299, 300, 302, 304, 306, 307, 308, 310, 311, 313, 315, 316, 317, 318, 319, 320, 322, 324, 326, 328, 330, 331, 333, 335, 337, 338, 340, 342, 343, 345, 346, 347, 349, 350, 351, 353, 354, 355, 357, 358, 360, 362, 363, 364, 365, 367, 369, 370, 371, 372, 373, 374, 375, 377, 378, 379, 380, 382, 383, 384, 385, 386, 387, 389, 390, 391, 393, 395, 396, 398, 400, 401, 403, 404, 406, 408, 409, 410, 411, 412, 414, 415, 417, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 456, 457, 459, 461, 462, 464, 466, 467, 468, 469, 470, 472, 474, 475, 477, 479, 480, 482, 483, 484, 485, 487, 488, 489, 490, 491, 492, 493, 494, 495, 497, 499, 501, 502, 503, 504, 506, 507, 509, 510, 511, 512, 514, 515, 516, 517, 518, 520, 522, 524, 526, 528, 530, 531, 532, 533, 534, 535, 536, 537, 538, 539, 540, 542, 543, 545, 546, 547, 548, 550, 551, 553, 554, 555, 557, 559, 560, 562, 563, 565, 566, 568, 570, 572, 573, 575, 576, 577, 579, 580, 581, 582, 583, 584, 585, 586, 587, 589, 590, 591, 592, 593, 595, 596, 597, 598, 599, 601, 602, 604, 605, 607, 609, 610, 611, 612, 613, 614, 615, 616, 617, 618, 619, 620, 621, 622, 623, 624, 625, 626, 627, 629, 631, 633, 634, 635, 36, 637, 638, 640, 642, 644, 646, 648, 650, 651, 653, 654, 655, 656, 657, 658, 659, 660, 662, 663, 664, 665, 666, 667, 669, 671, 672, 674, 676, 677, 679, 680, 682, 684, 685, 687, 689, 690, 691, 693, 694, 696, 697, 698, 00, 702, 704, 705, 706, 707, 709, 710, 712, 714, 715, 717, 718, 719, 720, 721, 722, 723, 725, 726, 727, 728, 730, 731, 732, 733, 734, 735, 737, 739, 741, 743, 744, 745, 746, 747, 749, 751, 752, 753, 755, 757, 759, 760, 762, 764, 766, 768, 770, 772, 773, 775, 776, 778, 780, 782, 784, 786, 788, 789, 791, 793, 794, 795, 796, 798, 800, 801, 802, 804, 805, 807, 809, 811, 813, 815, 817, 819, 820, 822, 823, 824, 825, 827, 828, 830, 832, 834, 836, 838, 840, 842, 843, 844, 846, 848, 849, 850, 851, 853, 855, 856, 857, 858, 860, 861, 863, 864, 866, 868, 870, 871, 873, 874, 876, 878, 880, 882, 883, 885, 887, 889, 890, 892, 893, 895, 896, 897, 899, 901, 902, 903, 905, 907, 908, 909, 910, 912, 914, 915, 916, 917, 919, 921, 923, 925, 926, 928, 930, 932, 934, 935, 936, 937, 939, 941, 943, 945, 947, 949, 950, 952, 953, 955, 957, 959, 960, 962, 964, 965, 967, 968, 970, 972, 974, 976, 978, 980, 981, 983, 985, 86, 87, 989, 991, 993, 995, 997, 998, 1000, 1002, 1003, 1005, 1007, 1008, 1009, 1011, 1013, 1014, 1016, 1018, 1020, 1022, 1023, 1024, 1025, 1026, 1027, 1029, 1031, 1033, 1034, 1036, 1038, 1039, 1041, 1042, 1043, 1044, 1045, 1046, 1048, 1049, 1050, 1051, 1052, 1054, 1056, 1058, 1060, 1061, 1062, 1064, 1066, 1067, 1069, 1070, 1071, 1072, 1074, 1076, 1077, 1078, 1079, 1081, 1083, 1085, 1087, 1088, 1090, 1092, 1093, 1095, 1096, 1097, 1099, 1101, 1102, 1104, 1105, 1107, 1108, 1110, 1111, 1112, 1113, 1114, 1115, 1117, 1119, 1120, 1121, 1122, 1124, 1126, 1128, 1130, 1132, 1133, 1135, 1137, 1139, 1141, 1142, 1144, 1146, 1148, 1150, 1152, 1154, 1156, 1157, 1158, 1160, 1162, 1164, 1166, 1168, 1169, 1171, 1173, 1175, 1176, 1177, 1179, 1180, 1181, 1183, 1185, 1187, 1188, 1190, 1192, 1193, 1194, 1196, 1198, 1200, 1201, 1203, 1205, 1207, 1209, 1210, 1212, 1214, 1216, 1218, 1219, 1221, 1222, 1224, 1226, 1228, 1230, 1232, 1234, 1235, 1236, 1238, 1240, 1241, 1243, 1245, 1247, 1249, 1251, 1252, 1253, 1255, 1257, 1259, 1261, 1263, 1265, 1266, 1268, 1270, 1272, 1274, 1276, 1278, 1279, 1281, 1283, 1285, 1287, 1289, 1291, 1293, 1294, 1296, 1297, 1299, 1300, 1302, 1304, 1306, 1308, 1309, 1310, 1312, 1313, 1314, 1316, 1318, 1320, 1322, 1324, 1325, 1326, 1328, 1329, 1330, 1332, 1333, 1334, 1336, 1338, 1339, 1340, 1341, 1342, 1343, 1344, 1345, 1347, 1349, 1350, 1352, 1353, 1355, 1357, 1358, 1359, 1360, 1361, 1363, 1364, 1366, 1367, 1369, 1370, 1371, 1372, 1373, 1375, 1376, 1377, 1379, 1380, 1381, 1382, 1384, 1386, 1388, 1389, 1391, 1393, 1395, 1397, 1399, 1401, 1402, 1403, 1405, 1407, 1409, 1410, 1412, 1414, 1416, 1418, 1420, 1421, 1422, 1424, 1426, 1428, 1429, 1431, 1433, 1435, 1437, 1439, 1440, 1441, 1443, 1445, 1446, 1448, 1450, 1452, 1454, 1456, 1457, 1459, 1461, 1463, 1465, 1467, 1469, 1471, 1473, 1475, 1477, 1479, 1481, 1482, 1483, 1485, 1486, 1488, 1489, 1490, 1492, 1493, 1495, 1497, 1498, 1500, 1502, 1503, 1505, 1506, 1507, 1508, 1509, 1511, 1512, 1514, 1515, 1516, 1518, 1520, 1522, 1523, 1524, 1525, 1526, 1528, 1529, 1531, 1533, 1534, 1535, 1537, 1538, 1540, 1542, 1544, 1546, 1547, 1549, 1550, 1552, 1554, 1555, 1557, 1558, 1560, 1562, 1563, 1564, 1565, 1568, 1569, 1571, 1572, 1574, 1575, 1577, 1578, 1580, 1581, 1583, 1584, 1586, 1588, 1589, 1591, 1593, 1594, 1595, 1596, 1598, 1599, 1600, 1602, 1603, 1604, 1605, 1607, 1609, 1611, 1612, 1613, 1615, 1616, 1618, 1619, 1621, 1622, 1624, 1626, 1628, 1629, 1630, 1632, 1634, 1636, 1637, 1639, 1640, 1642, 1644, 1646, 1647, 1648, 1649, 1650, 1652, 1654, 1656, 1657, 1658, 1660, 1661, 1662, 1664, 1666, 1668, 1669, 1670, 1671, 1672, 1674, 1676, 1677, 1679, 1680, 1681, 1682, 1683, 1684, 1686, 1688, 1689, 1691, 1693, 1695, 1697, 1699, 1700, 1701, 1702, 1704, 1705, 1706, 1707, 1708, 1710, 1711, 1712, 1713, 1714, 1715, 1717, 1719, 1721, 1723, 1724, 1725, 1727, 1729, 1731, 1732, 1734, 1735, 1737, 1739, 1741, 1743, 1745, 1746, 1747, 1749, 1751, 1753, 1755, 1757, 1758, 1759, 1761, 1762, 1764, 1765, 1766, 1767, 1769, 1770, 1772, 1773, 1774, 1776, 1777, 1779, 1781, 1783, 1784, 1786, 1788, 1790, 1792, 1794, 1795, 1797, 798, 1799, 1801, 1803, 1804, 1805, 1806, 1807, 1808, 1809, 1810, 1811, 1812, 1813, 1815, 1816, 1817, 1819, 1821, 1822, 1823, 1825, 1826, 1827, 1828, 1829, 1831, 1833, 1834, 1835, 1837, 1839, 1841, 1842, 1844, 1845, 1847, 1849, 1850, 1852, 1853, 1854, 1855, 1856, 1857, 1858, 1859, 1861, 1863, 1865, 1866, 1867, 1869, 1871, 1872, 1874, 1876, 1877, 1879, 1881, 1882, 1883, 1885, 1887, 1889, 1891, 1893, 1894, 1895, 1897, 1898, 1900, 1902, 1903, 1904, 1906, 1908, 1910, 1912, 1913, 1915, 1917, 1918, 1919, 1920, 1922, 1924, 1926, 1928, 1929, 1931, 1933, 1935, 1936, 1938, 1940, 1942, 1944, 1946, 1948, 1949, 1951, 1953, 1955, 1957, 1959, 1961, 1963, 1965, 1967, 1969, 1971, 1972, 1973, 1974, 1975, 1976, 1978, 1979, 1980, 1982, 1983, 1985, 1986, 1987, 1989, 1990, 1992, 1994, 1996, 1997, 1999, 2000, 2002, 2003, 2005, 2007, 2008, 2009, 2010, 2011, 2013, 2015, 2016, 2017, 2018, 2019, 2021, 2022, 2023, 2024, 2026, 2028, 2030, 2031, 2032, 2034, 2036, 2037, 2038, 2040, 2041, 2042, 2043, 2044, 2046, 2047, 2048, 2049, 2051, 2053, 2055, 2056, 2058, 2060, 2062, 2064, 2066, 2067, 2069, 2070, 2072, 2073, 2074, 2075, 2076, 2077, 2078, 2079, 2081, 2083, 2085, 2087, 2088, 2090, 2091, 2092, 2093, 2095, 2097, 2098, 2100, 2102, 2104, 2105, 2106, 2108, 2110, 2112, 2113, 2115, 2117, 2118, 2119, 2120, 2121, 2122, 2124, 2126, 2128, 2129, 2130, 2132, 2133, 2135, 2136, 2137, 2138, 2140, 2141, 2142, 2143, 2144, 2146, 2148, 2149, 2150, 2151, 2152, 2153, 2154, 2156, 2158, 2159, 2160, 2161, 2162, 2163, 2165, 2167, 2169, 2170, 2171, 2172, 2173, 2174, 2175, 2176, 2177, 2178, 2179, 2181, 2183, 2184, 2186, 2187, 2188, 2190, 2192, 2194, 2195, 2196, 2197, 2198, 2199, 2200, 2201, 2202, 2203, 2205, 2207, 2209, 2211, 2213, 2215, 2217, 2219, 2221, 2223, 2225, 2226, 2228, 2229, 2231, 2233, 2235, 2236, 2237, 2238, 2239, 2240, 2241, 2243, 2244, 2246, 2248, 2250, 2251, 2253, 2255, 2257, 2259, 2261, 2262, 2264, 2265, 2267, 2269, 2270, 2272, 2273, 2275, 2276, 2278, 2279, 2281, 2282, 2284, 2285, 2286, 2288, 2289, 2291, 2293, 2295, 2296, 2297, 2298, 2299, 2300, 2302, 2304, 2306, 2307, 2309, 2310, 2312, 2313, 2314, 2315, 2316, 2317, 2318, 2320, 2321, 2323, 2325, 2327, 2329, 2330, 2332, 2334, 2335, 2337, 2339, 2341, 2343, 2345, 2346, 2347, 2348, 2350, 2352, 2354, 2356, 2357, 2358, 2360, 2361, 2362, 2364, 2366, 2367, 2369, 2371, 2373, 2375, 2376, 2378, 2380, 2381, 2383, 2384, 2386, 2388, 2390, 2391, 2392, 2394, 2396, 2397, 2399, 2401, 2402, 2403, 2405, 2407, 2408, 2409, 2411, 2413, 2415, 2417, 2418, 2419, 2421, 2422, 2423, 2424, 2425, 2427, 2428, 2429, 2430, 2432, 2434, 2435, 2437, 2439, 2440, 2441, 2442, 2443, 2445, 2446, 2448, 2449, 2451, 2452, 2454, 2456, 2457, 2458, 2459, 2460, 2461, 2462, 2463, 2464, 2466, 2468, 2469, 2470, 2471, 2472, 2474, 2475, 2476, 2477, 2478, 2480, 2482, 2483, 2484, 2485, 2486, 2487, 2488, 2489, 2490, 2491, 2493, 2494, 2495, 2496, 2497, 2498, 2499, 2500, 2502, 2503, 2505, 2506, 2507, 2509, 2510, 2511, 2512, 2513, 2514, 2516, 2517, 2518, 2519, 2521, 2522, 2523, 2525, 2527, 2529, 2530, 2531, 2533, 2535, 2537, 2539, 2540, 2541, 2542, 2544, 2546, 2547, 2548, 2550, 2551, 2552, 2554, 2556, 2557, 2558, 2559, 2561, 2562, 2564, 2565, 2567, 2569, 2571, 2572, 2573, 2574, 2575, 2576, 2578, 2580, 2581, 2583, 2584, 2585, 2587, 2588, 2589, 2590, 2592, 2593, 2594, 2595, 2597, 2599, 2601, 2603, 2604, 2606, 2608, 2609, 2611, 2612, 2614, 2615, 2616, 2617, 2619, 2621, 2623, 2624, 2626, 2627, 2628, 2629, 2631, 2632, 2634, 2636, 2638, 2640, 2641, 2642, 2644, 2646, 2647, 2648, 2649, 2650, 2652, 2654, 2656, 2658, 2659, 2661, 2663, 2665, 2667, 2668, 2670, 2672, 2674, 2675, 2677, 2679, 2681, 2683, 2685, 2687, 2689, 2691, 2693, 2695, 2697, 2699, 2701, 2703, 2704, 2706, 2708, 2710, 2711, 2712, 2714, 2716, 2717, 2719, 2721, 2722, 2724, 2726, 2728, 2730, 2732, 2734, 2735, 2736, 2738, 2739, 2740, 2741, 2743, 2745, 2746, 2748, 2750, 2751, 2753, 2754, 2756, 2758, 2760, 2762, 2764, 2766, 2768, 2770, 2771, 2772, 2773, 2775, 2777, 2778, 2780, 2781, 2782, 2784, 2786, 2788, 2790, 2791, 2792, 2794, 2796, 2797, 2799, 2801, 2802, 2803, 2804, 2806, 2807, 2809, 2810, 2811, 2813, 2815, 2816, 2818, 2819, 2821, 2822, 2823, 2825, 2826, 2828, 2830, 2832, 2833, 2835, 2836, 2838, 2840, 2842, 2844, 2845, 2846, 2847, 2849, 2850, 2851, 2852, 2853, 2855, 2856, 2857, 2858, 2859, 2861, 2862, 2863, 2864, 2866, 2867, 2868, 2869, 2870, 2872, 2873, 2875, 2876, 2877, 2878, 2880, 2882, 2883, 2885, 2886, 2888, 2890, 2891, 2892, 2893, 2894, 2896, 2898, 2900, 2902, 2903, 2904, 2906, 2907, 2908, 2909, 2910, 2911, 2915, 2917, 2918, 2919, 2920, 2921, 2922, 2923, 2924 and 2926.

This document also features a method of producing a plant. The method includes growing a plant cell containing a modified endogenous nucleic acid encoding a polypeptide, wherein the HMM bit score of the amino acid sequence of the polypeptide is greater than about 65, the HMM based on the amino acid sequences depicted in one of FIGS. 1-50, and wherein the plant has a difference in the level of biomass as compared to the corresponding level of a control plant where said nucleic acid has not been modified.

In another aspect, this document features a plant cell containing a modified endogenous nucleic acid encoding a polypeptide, wherein the HMM bit score of the amino acid sequence of the polypeptide is greater than about 65, the HMM based on the amino acid sequences depicted in one of FIGS. 1-50, and wherein a plant produced from the plant cell has a difference in the level of biomass as compared to the corresponding level of a control plant where the nucleic acid has not been modified.

In another aspect, this document features a plant cell containing a modified biomass-modulating endogenous nucleic acid, the nucleic acid comprising a nucleotide sequence with an open reading frame having 80 percent or greater sequence identity to the nucleotide sequence selected from the group consisting of SEQ ID NO: 1, 3, 7, 9, 11, 14, 16, 19, 21, 23, 28, 31, 33, 36, 40, 42, 44, 46, 48, 50, 52, 54, 58, 61, 66, 68, 72, 76, 78, 87, 89, 114, 116, 118, 125, 127, 130, 132, 134, 136, 138, 143, 145, 151, 153, 156, 159, 161, 164, 166, 168, 170, 172, 174, 177, 179, 181, 184, 186, 188, 190, 192, 202, 204, 206, 210, 212, 216, 221, 225, 227, 230, 241, 243, 253, 280, 283, 285, 290, 292, 296, 298, 301, 303, 305, 309, 312, 314, 321, 323, 325, 327, 329, 332, 334, 336, 339, 341, 344, 348, 352, 356, 359, 361, 366, 368, 376, 381, 388, 392, 394, 397, 399, 402, 405, 407, 413, 416, 418, 455, 458, 460, 463, 465, 471, 473, 476, 478, 481, 486, 496, 498, 500, 505, 508, 513, 519, 521, 523, 525, 527, 529, 541, 544, 549, 552, 556, 558, 561, 564, 567, 569, 571, 574, 578, 588, 594, 600, 603, 606, 608, 628, 630, 632, 639, 641, 643, 645, 647, 649, 652, 661, 668, 670, 673, 675, 678, 681, 683, 686, 688, 692, 695, 699, 701, 703, 708, 711, 713, 716, 724, 729, 736, 738, 740, 742, 748, 750, 754, 756, 758, 761, 763, 765, 767, 769, 771, 774, 777, 779, 781, 783, 785, 787, 790, 792, 797, 799, 803, 806, 808, 810, 812, 814, 816, 818, 821, 826, 829, 831, 833, 835, 837, 839, 841, 845, 847, 852, 854, 859, 862, 865, 867, 869, 872, 875, 877, 879, 881, 884, 886, 888, 891, 894, 898, 900, 904, 906, 911, 913, 918, 920, 922, 924, 927, 929, 931, 933, 938, 940, 942, 944, 946, 948, 951, 954, 956, 958, 961, 963, 966, 969, 971, 973, 975, 977, 979, 982, 984, 988, 990, 992, 994, 996, 999, 1001, 1004, 1006, 1010, 1012, 1015, 1017, 1019, 1021, 1028, 1030, 1032, 1035, 1037, 1040, 1047, 1053, 1055, 1057, 1059, 1063, 1065, 1068, 1073, 1075, 1080, 1082, 1084, 1086, 1089, 1091, 1094, 1098, 1100, 1103, 1106, 1109, 1116, 1118, 1123, 1125, 1127, 1129, 1131, 1134, 1136, 1138, 1140, 1143, 1145, 1147, 1149, 1151, 1153, 1155, 1159, 1161, 1163, 1165, 1167, 1170, 1172, 1174, 1178, 1182, 1184, 1186, 1189, 1191, 1195, 1197, 1199, 1202, 1204, 1206, 1208, 1211, 1213, 1215, 1217, 1220, 1223, 1225, 1227, 1229, 1231, 1233, 1237, 1239, 1242, 1244, 1246, 1248, 1250, 1254, 1256, 1258, 1260, 1262, 1264, 1267, 1269, 1271, 1273, 1275, 1277, 1280, 1282, 1284, 1286, 1288, 1290, 1292, 1295, 1298, 1301, 1303, 1305, 1307, 1311, 1315, 1317, 1319, 1321, 1323, 1327, 1331, 1335, 1337, 1346, 1348, 1351, 1354, 1356, 1362, 1365, 1368, 1374, 1378, 1383, 1385, 1387, 1390, 1392, 1394, 1396, 1398, 1400, 1404, 1406, 1408, 1411, 1413, 1415, 1417, 1419, 1423, 1425, 1427, 1430, 1432, 1434, 1436, 1438, 1442, 1444, 1447, 1449, 1451, 1453, 1455, 1458, 1460, 1462, 1464, 1466, 1468, 1470, 1472, 1474, 1476, 1478, 1480, 1484, 1487, 1491, 1494, 1496, 1499, 1501, 1504, 1510, 1513, 1517, 1519, 1521, 1527, 1530, 1532, 1536, 1539, 1541, 1543, 1545, 1548, 1551, 1553, 1556, 1559, 1561, 1566, 1568, 1570, 1573, 1576, 1579, 1582, 1585, 1587, 1590, 1592, 1597, 1601, 1606, 1608, 1610, 1614, 1617, 1620, 1623, 1625, 1627, 1631, 1633, 1635, 1638, 1641, 1643, 1645, 1651, 1653, 1655, 1659, 1663, 1665, 1667, 1673, 1675, 1678, 1685, 1687, 1690, 1692, 1694, 1696, 1698, 1703, 1709, 1716, 1718, 1720, 1722, 1726, 1728, 1730, 1733, 1736, 1738, 1740, 1742, 1744, 1748, 1750, 1752, 1754, 1756, 1760, 1763, 1768, 1771, 1775, 1778, 1780, 1782, 1785, 1787, 1789, 1791, 1793, 1796, 1800, 1802, 1814, 1818, 1820, 1824, 1830, 1832, 1836, 1838, 1840, 1843, 1846, 1848, 1851, 1860, 1862, 1864, 1868, 1870, 1873, 1875, 1878, 1880, 1884, 1886, 1888, 1890, 1892, 1896, 1899, 1901, 1905, 1907, 1909, 1911, 1914, 1916, 1921, 1923, 1925, 1927, 1930, 1932, 1934, 1937, 1939, 1941, 1943, 1945, 1947, 1950, 1952, 1954, 1956, 1958, 1960, 1962, 1964, 1966, 1968, 1970, 1977, 1981, 1984, 1988, 1991, 1993, 1995, 1998, 2001, 2004, 2006, 2012, 2014, 2020, 2025, 2027, 2029, 2033, 2035, 2039, 2045, 2050, 2052, 2054, 2057, 2059, 2061, 2063, 2065, 2068, 2071, 2080, 2082, 2084, 2086, 2089, 2094, 2096, 2099, 2101, 2103, 2107, 2109, 2111, 2114, 2116, 2123, 2125, 2127, 2131, 2134, 2139, 2145, 2147, 2155, 2157, 2164, 2166, 2168, 2180, 2182, 2185, 2189, 2191, 2193, 2204, 2206, 2208, 2210, 2212, 2214, 2216, 2218, 2220, 2222, 2224, 2227, 2230, 2232, 2234, 2242, 2245, 2247, 2249, 2252, 2254, 2256, 2258, 2260, 2263, 2266, 2268, 2271, 2274, 2277, 2280, 2283, 2287, 2290, 2292, 2294, 2301, 2303, 2305, 2308, 2311, 2319, 2322, 2324, 2326, 2328, 2331, 2333, 2336, 2338, 2340, 2342, 2344, 2349, 2351, 2353, 2355, 2359, 2363, 2365, 2368, 2370, 2372, 2374, 2377, 2379, 2382, 2385, 2387, 2389, 2393, 2395, 2398, 2400, 2404, 2406, 2410, 2412, 2414, 2416, 2420, 2426, 2431, 2433, 2436, 2438, 2444, 2447, 2450, 2453, 2455, 2465, 2467, 2473, 2479, 2481, 2492, 2501, 2504, 2508, 2515, 2520, 2524, 2526, 2528, 2532, 2534, 2536, 2538, 2543, 2545, 2549, 2553, 2555, 2560, 2563, 2566, 2568, 2570, 2577, 2579, 2582, 2586, 2591, 2596, 2598, 2600, 2602, 2605, 2607, 2610, 2613, 2618, 2620, 2622, 2625, 2630, 2633, 2635, 2637, 2639, 2643, 2645, 2651, 2653, 2655, 2657, 2660, 2662, 2664, 2666, 2669, 2671, 2673, 2676, 2678, 2680, 2682, 2684, 2686, 2688, 2690, 2692, 2694, 2696, 2698, 2700, 2702, 2705, 2707, 2709, 2713, 2715, 2718, 2720, 2723, 2725, 2727, 2729, 2731, 2733, 2737, 2742, 2744, 2747, 2749, 2752, 2755, 2757, 2759, 2761, 2763, 2765, 2767, 2769, 2774, 2776, 2779, 2783, 2785, 2787, 2789, 2793, 2795, 2798, 2800, 2805, 2808, 2812, 2814, 2817, 2820, 2824, 2827, 2829, 2831, 2834, 2837, 2839, 2841, 2843, 2848, 2854, 2860, 2865, 2871, 2874, 2879, 2881, 2884, 2887, 2889, 2895, 2897, 2899, 2901, 2905, 2912, 2914, 2916, and 2925, and wherein a plant produced from the plant cell has a difference in the level of biomass as compared to the corresponding level of a control plant where the nucleic acid has not been modified.

In the plant cells described herein, the endogenous nucleic acid can encode a polypeptide having 80 percent or greater sequence identity to an amino acid sequence selected from the group consisting of SEQ ID NO: 2, 4, 5, 6, 8, 10, 12, 13, 15, 17, 18, 20, 22, 24, 25, 26, 27, 29, 30, 32, 34, 35, 37, 38, 39, 41, 43, 45, 47, 49, 51, 53, 55, 56, 57, 59, 60, 62, 63, 64, 65, 67, 69, 70, 71, 73, 74, 75, 77, 79, 80, 81, 82, 83, 84, 85, 86, 88, 90, 91, 92, 93, 94, 95, 96, 97, 98, 99, 100, 101, 102, 103, 104, 105, 106, 107, 108, 109, 110, 111, 112, 113, 115, 117, 119, 120, 121, 122, 123, 124, 126, 128, 129, 131, 133, 135, 137, 139, 140, 141, 142, 144, 146, 147, 148, 149, 150, 152, 154, 155, 157, 158, 160, 162, 163, 165, 167, 169, 171, 173, 175, 176, 178, 180, 182, 183, 185, 187, 189, 191, 193, 194, 195, 196, 197, 198, 199, 200, 201, 203, 205, 207, 208, 209, 211, 213, 214, 215, 217, 218, 219, 220, 222, 223, 224, 226, 228, 229, 231, 232, 233, 234, 235, 236, 237, 238, 239, 240, 242, 244, 245, 246, 247, 248, 249, 250, 251, 252, 254, 255, 256, 257, 258, 259, 260, 261, 262, 263, 264, 265, 266, 267, 268, 269, 270, 271, 272, 273, 274, 275, 276, 277, 278, 279, 281, 282, 284, 286, 287, 288, 289, 291, 293, 294, 295, 297, 299, 300, 302, 304, 306, 307, 308, 310, 311, 313, 315, 316, 317, 318, 319, 320, 322, 324, 326, 328, 330, 331, 333, 335, 337, 338, 340, 342, 343, 345, 346, 347, 349, 350, 351, 353, 354, 355, 357, 358, 360, 362, 363, 364, 365, 367, 369, 370, 371, 372, 373, 374, 375, 377, 378, 379, 380, 382, 383, 384, 385, 386, 387, 389, 390, 391, 393, 395, 396, 398, 400, 401, 403, 404, 406, 408, 409, 410, 411, 412, 414, 415, 417, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 456, 457, 459, 461, 462, 464, 466, 467, 468, 469, 470, 472, 474, 475, 477, 479, 480, 482, 483, 484, 485, 487, 488, 489, 490, 491, 492, 493, 494, 495, 497, 499, 501, 502, 503, 504, 506, 507, 509, 510, 511, 512, 514, 515, 516, 517, 518, 520, 522, 524, 526, 528, 530, 531, 532, 533, 534, 535, 536, 537, 538, 539, 540, 542, 543, 545, 546, 547, 548, 550, 551, 553, 554, 555, 557, 559, 560, 562, 563, 565, 566, 568, 570, 572, 573, 575, 576, 577, 579, 580, 581, 582, 583, 584, 585, 586, 587, 589, 590, 591, 592, 593, 595, 596, 597, 598, 599, 601, 602, 604, 605, 607, 609, 610, 611, 612, 613, 614, 615, 616, 617, 618, 619, 620, 621, 622, 623, 624, 625, 626, 627, 629, 631, 633, 634, 635, 36, 637, 638, 640, 642, 644, 646, 648, 650, 651, 653, 654, 655, 656, 657, 658, 659, 660, 662, 663, 664, 665, 666, 667, 669, 671, 672, 674, 676, 677, 679, 680, 682, 684, 685, 687, 689, 690, 691, 693, 694, 696, 697, 698, 00, 702, 704, 705, 706, 707, 709, 710, 712, 714, 715, 717, 718, 719, 720, 721, 722, 723, 725, 726, 727, 728, 730, 731, 732, 733, 734, 735, 737, 739, 741, 743, 744, 745, 746, 747, 749, 751, 752, 753, 755, 757, 759, 760, 762, 764, 766, 768, 770, 772, 773, 775, 776, 778, 780, 782, 784, 786, 788, 789, 791, 793, 794, 795, 796, 798, 800, 801, 802, 804, 805, 807, 809, 811, 813, 815, 817, 819, 820, 822, 823, 824, 825, 827, 828, 830, 832, 834, 836, 838, 840, 842, 843, 844, 846, 848, 849, 850, 851, 853, 855, 856, 857, 858, 860, 861, 863, 864, 866, 868, 870, 871, 873, 874, 876, 878, 880, 882, 883, 885, 887, 889, 890, 892, 893, 895, 896, 897, 899, 901, 902, 903, 905, 907, 908, 909, 910, 912, 914, 915, 916, 917, 919, 921, 923, 925, 926, 928, 930, 932, 934, 935, 936, 937, 939, 941, 943, 945, 947, 949, 950, 952, 953, 955, 957, 959, 960, 962, 964, 965, 967, 968, 970, 972, 974, 976, 978, 980, 981, 983, 985, 86, 87, 989, 991, 993, 995, 997, 998, 1000, 1002, 1003, 1005, 1007, 1008, 1009, 1011, 1013, 1014, 1016, 1018, 1020, 1022, 1023, 1024, 1025, 1026, 1027, 1029, 1031, 1033, 1034, 1036, 1038, 1039, 1041, 1042, 1043, 1044, 1045, 1046, 1048, 1049, 1050, 1051, 1052, 1054, 1056, 1058, 1060, 1061, 1062, 1064, 1066, 1067, 1069, 1070, 1071, 1072, 1074, 1076, 1077, 1078, 1079, 1081, 1083, 1085, 1087, 1088, 1090, 1092, 1093, 1095, 1096, 1097, 1099, 1101, 1102, 1104, 1105, 1107, 1108, 1110, 1111, 1112, 1113, 1114, 1115, 1117, 1119, 1120, 1121, 1122, 1124, 1126, 1128, 1130, 1132, 1133, 1135, 1137, 1139, 1141, 1142, 1144, 1146, 1148, 1150, 1152, 1154, 1156, 1157, 1158, 1160, 1162, 1164, 1166, 1168, 1169, 1171, 1173, 1175, 1176, 1177, 1179, 1180, 1181, 1183, 1185, 1187, 1188, 1190, 1192, 1193, 1194, 1196, 1198, 1200, 1201, 1203, 1205, 1207, 1209, 1210, 1212, 1214, 1216, 1218, 1219, 1221, 1222, 1224, 1226, 1228, 1230, 1232, 1234, 1235, 1236, 1238, 1240, 1241, 1243, 1245, 1247, 1249, 1251, 1252, 1253, 1255, 1257, 1259, 1261, 1263, 1265, 1266, 1268, 1270, 1272, 1274, 1276, 1278, 1279, 1281, 1283, 1285, 1287, 1289, 1291, 1293, 1294, 1296, 1297, 1299, 1300, 1302, 1304, 1306, 1308, 1309, 1310, 1312, 1313, 1314, 1316, 1318, 1320, 1322, 1324, 1325, 1326, 1328, 1329, 1330, 1332, 1333, 1334, 1336, 1338, 1339, 1340, 1341, 1342, 1343, 1344, 1345, 1347, 1349, 1350, 1352, 1353, 1355, 1357, 1358, 1359, 1360, 1361, 1363, 1364, 1366, 1367, 1369, 1370, 1371, 1372, 1373, 1375, 1376, 1377, 1379, 1380, 1381, 1382, 1384, 1386, 1388, 1389, 1391, 1393, 1395, 1397, 1399, 1401, 1402, 1403, 1405, 1407, 1409, 1410, 1412, 1414, 1416, 1418, 1420, 1421, 1422, 1424, 1426, 1428, 1429, 1431, 1433, 1435, 1437, 1439, 1440, 1441, 1443, 1445, 1446, 1448, 1450, 1452, 1454, 1456, 1457, 1459, 1461, 1463, 1465, 1467, 1469, 1471, 1473, 1475, 1477, 1479, 1481, 1482, 1483, 1485, 1486, 1488, 1489, 1490, 1492, 1493, 1495, 1497, 1498, 1500, 1502, 1503, 1505, 1506, 1507, 1508, 1509, 1511, 1512, 1514, 1515, 1516, 1518, 1520, 1522, 1523, 1524, 1525, 1526, 1528, 1529, 1531, 1533, 1534, 1535, 1537, 1538, 1540, 1542, 1544, 1546, 1547, 1549, 1550, 1552, 1554, 1555, 1557, 1558, 1560, 1562, 1563, 1564, 1565, 1568, 1569, 1571, 1572, 1574, 1575, 1577, 1578, 1580, 1581, 1583, 1584, 1586, 1588, 1589, 1591, 1593, 1594, 1595, 1596, 1598, 1599, 1600, 1602, 1603, 1604, 1605, 1607, 1609, 1611, 1612, 1613, 1615, 1616, 1618, 1619, 1621, 1622, 1624, 1626, 1628, 1629, 1630, 1632, 1634, 1636, 1637, 1639, 1640, 1642, 1644, 1646, 1647, 1648, 1649, 1650, 1652, 1654, 1656, 1657, 1658, 1660, 1661, 1662, 1664, 1666, 1668, 1669, 1670, 1671, 1672, 1674, 1676, 1677, 1679, 1680, 1681, 1682, 1683, 1684, 1686, 1688, 1689, 1691, 1693, 1695, 1697, 1699, 1700, 1701, 1702, 1704, 1705, 1706, 1707, 1708, 1710, 1711, 1712, 1713, 1714, 1715, 1717, 1719, 1721, 1723, 1724, 1725, 1727, 1729, 1731, 1732, 1734, 1735, 1737, 1739, 1741, 1743, 1745, 1746, 1747, 1749, 1751, 1753, 1755, 1757, 1758, 1759, 1761, 1762, 1764, 1765, 1766, 1767, 1769, 1770, 1772, 1773, 1774, 1776, 1777, 1779, 1781, 1783, 1784, 1786, 1788, 1790, 1792, 1794, 1795, 1797, 798, 1799, 1801, 1803, 1804, 1805, 1806, 1807, 1808, 1809, 1810, 1811, 1812, 1813, 1815, 1816, 1817, 1819, 1821, 1822, 1823, 1825, 1826, 1827, 1828, 1829, 1831, 1833, 1834, 1835, 1837, 1839, 1841, 1842, 1844, 1845, 1847, 1849, 1850, 1852, 1853, 1854, 1855, 1856, 1857, 1858, 1859, 1861, 1863, 1865, 1866, 1867, 1869, 1871, 1872, 1874, 1876, 1877, 1879, 1881, 1882, 1883, 1885, 1887, 1889, 1891, 1893, 1894, 1895, 1897, 1898, 1900, 1902, 1903, 1904, 1906, 1908, 1910, 1912, 1913, 1915, 1917, 1918, 1919, 1920, 1922, 1924, 1926, 1928, 1929, 1931, 1933, 1935, 1936, 1938, 1940, 1942, 1944, 1946, 1948, 1949, 1951, 1953, 1955, 1957, 1959, 1961, 1963, 1965, 1967, 1969, 1971, 1972, 1973, 1974, 1975, 1976, 1978, 1979, 1980, 1982, 1983, 1985, 1986, 1987, 1989, 1990, 1992, 1994, 1996, 1997, 1999, 2000, 2002, 2003, 2005, 2007, 2008, 2009, 2010, 2011, 2013, 2015, 2016, 2017, 2018, 2019, 2021, 2022, 2023, 2024, 2026, 2028, 2030, 2031, 2032, 2034, 2036, 2037, 2038, 2040, 2041, 2042, 2043, 2044, 2046, 2047, 2048, 2049, 2051, 2053, 2055, 2056, 2058, 2060, 2062, 2064, 2066, 2067, 2069, 2070, 2072, 2073, 2074, 2075, 2076, 2077, 2078, 2079, 2081, 2083, 2085, 2087, 2088, 2090, 2091, 2092, 2093, 2095, 2097, 2098, 2100, 2102, 2104, 2105, 2106, 2108, 2110, 2112, 2113, 2115, 2117, 2118, 2119, 2120, 2121, 2122, 2124, 2126, 2128, 2129, 2130, 2132, 2133, 2135, 2136, 2137, 2138, 2140, 2141, 2142, 2143, 2144, 2146, 2148, 2149, 2150, 2151, 2152, 2153, 2154, 2156, 2158, 2159, 2160, 2161, 2162, 2163, 2165, 2167, 2169, 2170, 2171, 2172, 2173, 2174, 2175, 2176, 2177, 2178, 2179, 2181, 2183, 2184, 2186, 2187, 2188, 2190, 2192, 2194, 2195, 2196, 2197, 2198, 2199, 2200, 2201, 2202, 2203, 2205, 2207, 2209, 2211, 2213, 2215, 2217, 2219, 2221, 2223, 2225, 2226, 2228, 2229, 2231, 2233, 2235, 2236, 2237, 2238, 2239, 2240, 2241, 2243, 2244, 2246, 2248, 2250, 2251, 2253, 2255, 2257, 2259, 2261, 2262, 2264, 2265, 2267, 2269, 2270, 2272, 2273, 2275, 2276, 2278, 2279, 2281, 2282, 2284, 2285, 2286, 2288, 2289, 2291, 2293, 2295, 2296, 2297, 2298, 2299, 2300, 2302, 2304, 2306, 2307, 2309, 2310, 2312, 2313, 2314, 2315, 2316, 2317, 2318, 2320, 2321, 2323, 2325, 2327, 2329, 2330, 2332, 2334, 2335, 2337, 2339, 2341, 2343, 2345, 2346, 2347, 2348, 2350, 2352, 2354, 2356, 2357, 2358, 2360, 2361, 2362, 2364, 2366, 2367, 2369, 2371, 2373, 2375, 2376, 2378, 2380, 2381, 2383, 2384, 2386, 2388, 2390, 2391, 2392, 2394, 2396, 2397, 2399, 2401, 2402, 2403, 2405, 2407, 2408, 2409, 2411, 2413, 2415, 2417, 2418, 2419, 2421, 2422, 2423, 2424, 2425, 2427, 2428, 2429, 2430, 2432, 2434, 2435, 2437, 2439, 2440, 2441, 2442, 2443, 2445, 2446, 2448, 2449, 2451, 2452, 2454, 2456, 2457, 2458, 2459, 2460, 2461, 2462, 2463, 2464, 2466, 2468, 2469, 2470, 2471, 2472, 2474, 2475, 2476, 2477, 2478, 2480, 2482, 2483, 2484, 2485, 2486, 2487, 2488, 2489, 2490, 2491, 2493, 2494, 2495, 2496, 2497, 2498, 2499, 2500, 2502, 2503, 2505, 2506, 2507, 2509, 2510, 2511, 2512, 2513, 2514, 2516, 2517, 2518, 2519, 2521, 2522, 2523, 2525, 2527, 2529, 2530, 2531, 2533, 2535, 2537, 2539, 2540, 2541, 2542, 2544, 2546, 2547, 2548, 2550, 2551, 2552, 2554, 2556, 2557, 2558, 2559, 2561, 2562, 2564, 2565, 2567, 2569, 2571, 2572, 2573, 2574, 2575, 2576, 2578, 2580, 2581, 2583, 2584, 2585, 2587, 2588, 2589, 2590, 2592, 2593, 2594, 2595, 2597, 2599, 2601, 2603, 2604, 2606, 2608, 2609, 2611, 2612, 2614, 2615, 2616, 2617, 2619, 2621, 2623, 2624, 2626, 2627, 2628, 2629, 2631, 2632, 2634, 2636, 2638, 2640, 2641, 2642, 2644, 2646, 2647, 2648, 2649, 2650, 2652, 2654, 2656, 2658, 2659, 2661, 2663, 2665, 2667, 2668, 2670, 2672, 2674, 2675, 2677, 2679, 2681, 2683, 2685, 2687, 2689, 2691, 2693, 2695, 2697, 2699, 2701, 2703, 2704, 2706, 2708, 2710, 2711, 2712, 2714, 2716, 2717, 2719, 2721, 2722, 2724, 2726, 2728, 2730, 2732, 2734, 2735, 2736, 2738, 2739, 2740, 2741, 2743, 2745, 2746, 2748, 2750, 2751, 2753, 2754, 2756, 2758, 2760, 2762, 2764, 2766, 2768, 2770, 2771, 2772, 2773, 2775, 2777, 2778, 2780, 2781, 2782, 2784, 2786, 2788, 2790, 2791, 2792, 2794, 2796, 2797, 2799, 2801, 2802, 2803, 2804, 2806, 2807, 2809, 2810, 2811, 2813, 2815, 2816, 2818, 2819, 2821, 2822, 2823, 2825, 2826, 2828, 2830, 2832, 2833, 2835, 2836, 2838, 2840, 2842, 2844, 2845, 2846, 2847, 2849, 2850, 2851, 2852, 2853, 2855, 2856, 2857, 2858, 2859, 2861, 2862, 2863, 2864, 2866, 2867, 2868, 2869, 2870, 2872, 2873, 2875, 2876, 2877, 2878, 2880, 2882, 2883, 2885, 2886, 2888, 2890, 2891, 2892, 2893, 2894, 2896, 2898, 2900, 2902, 2903, 2904, 2906, 2907, 2908, 2909, 2910, 2911, 2915, 2917, 2918, 2919, 2920, 2921, 2922, 2923, 2924 and 2926. A plant produced from such a plant cell has a difference in the level of biomass as compared to the corresponding level of a control plant where the nucleic acid has not been modified.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims. The details of one or more embodiments of the invention are set forth in the accompanying drawings and the description below. Other features, objects, and advantages of the invention will be apparent from the description and drawings, and from the claims. Applicants reserve the right to alternatively claim any disclosed invention using the transitional phrase “comprising,” “consisting essentially of,” or “consisting of,” according to standard practice in patent law.

### DESCRIPTION OF THE DRAWINGS

FIG. 1 is an alignment of the amino acid sequence of ME26166 corresponding to CeresAnnot:831967 (SEQ ID NO:2880) with homologous and/or orthologous amino acid sequences. In all the alignment figures shown herein, a dash in an aligned sequence represents a gap, i.e., a lack of an amino acid at that position. Identical amino acids or conserved amino acid substitutions among aligned sequences are identified by boxes. FIG. 1 and the other alignment figures provided herein were generated using the program MUSCLE version 3.52.

FIG. 2 is an alignment of the amino acid sequence corresponding to CeresAnnot:837136 (SEQ ID NO:310) with homologous and/or orthologous amino acid sequences.

FIG. 3 is an alignment of the amino acid sequence of ME03563 corresponding to CeresClone:5055 (SEQ ID NO:403) with homologous and/or orthologous amino acid sequences.

FIG. 4 is an alignment of the amino acid sequence of ME07523 or ME04241 corresponding to CeresClone:14432 (SEQ ID NO:669) with homologous and/or orthologous amino acid sequences.

FIG. 5 is an alignment of the amino acid sequence of ME25767, ME26161, or ME06492 corresponding to CeresClone:21240 (SEQ ID NO:1797) with homologous and/or orthologous amino acid sequences.

FIG. 6 is an alignment of the amino acid sequence of ME24912 corresponding to CeresClone:570057 (SEQ ID NO:717) with homologous and/or orthologous amino acid sequences.

FIG. 7 is an alignment of the amino acid sequence of ME05057 corresponding to CeresClone:691319 (SEQ ID NO:693) with homologous and/or orthologous amino acid sequences.

FIG. 8 is an alignment of the amino acid sequence of ME29158 corresponding to CeresClone:1724055 (SEQ ID NO:1518) with homologous and/or orthologous amino acid sequences.

FIG. 9 is an alignment of the amino acid sequence of ME26627 corresponding to CeresClone:1724569 (SEQ ID NO:1624) with homologous and/or orthologous amino acid sequences.

FIG. 10 is an alignment of the amino acid sequence of ME26114 corresponding to CeresClone:1724782 (SEQ ID NO:1887) with homologous and/or orthologous amino acid sequences.

FIG. 11 is an alignment of the amino acid sequence corresponding to CeresClone:1724846 (SEQ ID NO:834) with homologous and/or orthologous amino acid sequences.

FIG. 12 is an alignment of the amino acid sequence of ME31528 corresponding to CeresClone:1725400 (SEQ ID NO:1318) with homologous and/or orthologous amino acid sequences.

FIG. 13 is an alignment of the amino acid sequence of ME29794 corresponding to CeresClone:1726076 (SEQ ID NO:459) with homologous and/or orthologous amino acid sequences.

FIG. 14 is an alignment of the amino acid sequence corresponding to CeresClone:1727475 (SEQ ID NO:335) with homologous and/or orthologous amino acid sequences.

FIG. 15 is an alignment of the amino acid sequence of ME27144 corresponding to CeresClone:1727628 (SEQ ID NO:846) with homologous and/or orthologous amino acid sequences.

FIG. 16 is an alignment of the amino acid sequence of ME31185 corresponding to CeresClone:1760764 (SEQ ID NO:1426) with homologous and/or orthologous amino acid sequences.

FIG. 17 is an alignment of the amino acid sequence of ME26843 corresponding to CeresClone:1763298 (SEQ ID NO:2525) with homologous and/or orthologous amino acid sequences.

FIG. 18 is an alignment of the amino acid sequence of ME31173 corresponding to CeresClone:1764605 (SEQ ID NO:1272) with homologous and/or orthologous amino acid sequences.

FIG. 19 is an alignment of the amino acid sequence corresponding to CeresClone:1767521 (SEQ ID NO:878) with homologous and/or orthologous amino acid sequences.

FIG. 20 is an alignment of the amino acid sequence corresponding to CeresClone:1769660 (SEQ ID NO:1660) with homologous and/or orthologous amino acid sequences.

FIG. 21 is an alignment of the amino acid sequence of ME31545 corresponding to CeresClone:1773290 (SEQ ID NO:2654) with homologous and/or orthologous amino acid sequences.

FIG. 22 is an alignment of the amino acid sequence corresponding to CeresClone:1775942 (SEQ ID NO:1137) with homologous and/or orthologous amino acid sequences.

FIG. 23 is an alignment of the amino acid sequence of ME30365 corresponding to CeresClone:1777248 (SEQ ID NO:2466) with homologous and/or orthologous amino acid sequences.

FIG. 24 is an alignment of the amino acid sequence of ME29764 corresponding to CeresClone:1781320 (SEQ ID NO:1107) with homologous and/or orthologous amino acid sequences.

FIG. 25 is an alignment of the amino acid sequence corresponding to CeresClone:1788006 (SEQ ID NO:1946) with homologous and/or orthologous amino acid sequences.

FIG. 26 is an alignment of the amino acid sequence of ME31183 corresponding to CeresClone:1788124 (SEQ ID NO:1054) with homologous and/or orthologous amino acid sequences.

FIG. 27 is an alignment of the amino acid sequence of ME30345 corresponding to CeresClone:1788820 (SEQ ID NO:115) with homologous and/or orthologous amino acid sequences.

FIG. 28 is an alignment of the amino acid sequence of ME29970 corresponding to CeresClone:1789564 (SEQ ID NO:2708) with homologous and/or orthologous amino acid sequences.

FIG. 29 is an alignment of the amino acid sequence of ME28415 corresponding to CeresClone:1789593 (SEQ ID NO:1481) with homologous and/or orthologous amino acid sequences.

FIG. 30 is an alignment of the amino acid sequence of ME29777 corresponding to CeresClone:1790340 (SEQ ID NO:2748) with homologous and/or orthologous amino acid sequences.

FIG. 31 is an alignment of the amino acid sequence of ME28947 corresponding to CeresClone:1790811 (SEQ ID NO:947) with homologous and/or orthologous amino acid sequences.

FIG. 32 is an alignment of the amino acid sequence of ME28309 corresponding to CeresClone:1792586 (SEQ ID NO:1727) with homologous and/or orthologous amino acid sequences.

FIG. 33 is an alignment of the amino acid sequence of ME27658 corresponding to CeresClone:1794784 (SEQ ID NO:2146) with homologous and/or orthologous amino acid sequences.

FIG. 34 is an alignment of the amino acid sequence of ME27043 corresponding to CeresClone:1797459 (SEQ ID NO:2619) with homologous and/or orthologous amino acid sequences.

FIG. 35 is an alignment of the amino acid sequence corresponding to CeresClone:1801750 (SEQ ID NO:211) with homologous and/or orthologous amino acid sequences.

FIG. 36 is an alignment of the amino acid sequence of ME26515 corresponding to CeresClone:1804242 (SEQ ID NO:629) with homologous and/or orthologous amino acid sequences.

FIG. 37 is an alignment of the amino acid sequence corresponding to CeresClone:1806608 (SEQ ID NO:2544) with homologous and/or orthologous amino acid sequences.

FIG. 38 is an alignment of the amino acid sequence corresponding to CeresClone:1809393 (SEQ ID NO:2599) with homologous and/or orthologous amino acid sequences.

FIG. 39 is an alignment of the amino acid sequence of ME25694 corresponding to CeresClone:1815770 (SEQ ID NO:2437) with homologous and/or orthologous amino acid sequences.

FIG. 40 is an alignment of the amino acid sequence corresponding to CeresClone:1817100 (SEQ ID NO:2090) with homologous and/or orthologous amino acid sequences.

FIG. 41 is an alignment of the amino acid sequence corresponding to CeresClone:1821162 (SEQ ID NO:2323) with homologous and/or orthologous amino acid sequences.

FIG. 42 is an alignment of the amino acid sequence of ME27358 corresponding to CeresClone:1824159 (SEQ ID NO:2) with homologous and/or orthologous amino acid sequences.

FIG. 43 is an alignment of the amino acid sequence corresponding to CeresClone:1830065 (SEQ ID NO:2028) with homologous and/or orthologous amino acid sequences.

FIG. 44 is an alignment of the amino acid sequence of ME30392 corresponding to CeresClone:1900097 (SEQ ID NO:1569) with homologous and/or orthologous amino acid sequences.

FIG. 45 is an alignment of the amino acid sequence corresponding to CeresClone:1902646 (SEQ ID NO:501) with homologous and/or orthologous amino acid sequences.

FIG. 46 is an alignment of the amino acid sequence of ME30330 corresponding to CeresClone:1955550 (SEQ ID NO:2190) with homologous and/or orthologous amino acid sequences.

FIG. 47 is an alignment of the amino acid sequence of ME29626 corresponding to CeresClone:1955766 (SEQ ID NO:749) with homologous and/or orthologous amino acid sequences.

FIG. 48 is an alignment of the amino acid sequence of ME30683 corresponding to CeresClone:2010191 (SEQ ID NO:79) with homologous and/or orthologous amino acid sequences.

FIG. 49 is an alignment of the amino acid sequence of ME30110 corresponding to CeresClone:2014404 (SEQ ID NO:284) with homologous and/or orthologous amino acid sequences.

FIG. 50 is an alignment of the amino acid sequence corresponding to CeresClone:1872280 (SEQ ID NO:2915) with homologous and/or orthologous amino acid sequences.

### DETAILED DESCRIPTION

The invention features methods and materials related to modulating biomass levels in plants. In some embodiments, the plants may also have modulated levels of, for example, lignin, modified root architecture, modified herbicide resistance, modified carotenoid biosynthesis, or modulated cell wall content. The methods can include transforming a plant cell with a nucleic acid encoding a biomass-modulating polypeptide, wherein expression of the polypeptide results in a modulated level of biomass. Plant cells produced using such methods can be grown to produce plants having an increased or decreased biomass. Such plants, and the seeds of such plants, may be used to produce, for example, biomass having an increased value as a biofuel feedstock.

### I. Definitions

“Amino acid” refers to one of the twenty biologically occurring amino acids and to synthetic amino acids, including D/L optical isomers.

“Biomass” refers to plant-derived organic matter. Biomass includes plant matter derived from herbaceous and woody energy crops, agricultural food and feed crops, agricultural crop wastes and residues, wood wastes and residues, aquatic plants, and other plant-derived materials including some municipal wastes. Biomass is a heterogeneous and chemically complex renewable resource.

“Biomass component” refers to a component found in a plant such as glucan, xylan, fermentable sugars, arabinan, sucrose, lignin, protein, ash, and extractives.

“Carbohydrate material” refers to carbon polymers found in biomass, such as glucans, arabinans, xylans, and pectins.

“Cellulose” refers to a glucan polysaccharide, i.e., a glucose polymer with β-1,4-glycosidic linkages. The β-linkages in cellulose form linear chains that are highly stable and resistant to chemical attack because of the high degree of hydrogen bonding that can occur between chains of cellulose. Hydrolysis of cellulose results in the production of cellobiose, C₁₂H₂₂O₁₁, and the monosaccharide glucose, C₆H₁₂O₆. Cellulose is the principal carbohydrate constituent of wood and other biomass.

“Cell type-preferential promoter” or “tissue-preferential promoter” refers to a promoter that drives expression preferentially in a target cell type or tissue, respectively, but may also lead to some transcription in other cell types or tissues as well.

“Control plant” refers to a plant that does not contain the exogenous nucleic acid present in a transgenic plant of interest, but otherwise has the same or similar genetic background as such a transgenic plant. A suitable control plant can be a non-transgenic wild type plant, a non-transgenic segregant from a transformation experiment, or a transgenic plant that contains an exogenous nucleic acid other than the exogenous nucleic acid of interest.

“Domains” are groups of substantially contiguous amino acids in a polypeptide that can be used to characterize protein families and/or parts of proteins. Such domains have a “fingerprint” or “signature” that can comprise conserved primary sequence, secondary structure, and/or three-dimensional conformation. Generally, domains are correlated with specific in vitro and/or in vivo activities. A domain can have a length of from 10 amino acids to 400 amino acids, e.g., 10 to 50 amino acids, or 25 to 100 amino acids, or 35 to 65 amino acids, or 35 to 55 amino acids, or 45 to 60 amino acids, or 200 to 300 amino acids, or 300 to 400 amino acids.

“Down-regulation” refers to regulation that decreases production of expression products (mRNA, polypeptide, or both) relative to basal or native states.

“Exogenous” with respect to a nucleic acid indicates that the nucleic acid is part of a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. It will be appreciated that an exogenous nucleic acid may have been introduced into a progenitor and not into the cell under consideration. For example, a transgenic plant containing an exogenous nucleic acid can be the progeny of a cross between a stably transformed plant and a non-transgenic plant. Such progeny are considered to contain the exogenous nucleic acid.

“Expression” refers to the process of converting genetic information of a polynucleotide into RNA through transcription, which is catalyzed by an enzyme, RNA polymerase, and into protein, through translation of mRNA on ribosomes.

“Glucan,” “Xylan” and “Arabinan” refer to the anhydro forms of glucose, xylose and arabinose that are found in cellulose and hemicellulose carbohydrate polymers. Thus, for example, “glucan” refers to a polysaccharide of D-glucose monomers linked by glycosidic bonds. The following are glucans: cellulose (β-1,4-glucan), dextran (α-1,6-glucan) and starch (α-1,4- and α-1,6-glucan).

“Hemicellulose” is a general term used to refer to cell wall polysaccharides that are not celluloses or pectins. Hemicelluloses contain repeating monomeric units of a five-carbon sugar (usually D-xylose or L-arabinose) and/or a six-carbon sugar (D-galactose, D-glucose, and D-mannose). See, U.S. Pat. No. 7,112,429. Hemicelluloses typically are shorter in length than cellulose and are highly branched. Xylan is often the structural backbone of hemicelluloses from hardwoods and grasses, and hydrolysis of these biomass types releases products high in the five-carbon sugar, xylose. Hemicelluloses from softwoods are most commonly gluco-galacto-mannans, which have a mannan backbone and yield mannose as the main product of hydrolysis. Hemicelluloses often contain side groups such as acetyl groups, uronic acids and ferulates.

“Heterologous polypeptide” as used herein refers to a polypeptide that is not a naturally occurring polypeptide in a plant cell, e.g., a transgenic Panicum virgatum plant transformed with and expressing the coding sequence for a nitrogen transporter polypeptide from a Zea mays plant.

“Isolated nucleic acid” as used herein includes a naturally-occurring nucleic acid, provided one or both of the sequences immediately flanking that nucleic acid in its naturally-occurring genome is removed or absent. Thus, an isolated nucleic acid includes, without limitation, a nucleic acid that exists as a purified molecule or a nucleic acid molecule that is incorporated into a vector or a virus. A nucleic acid existing among hundreds to millions of other nucleic acids within, for example, cDNA libraries, genomic libraries, or gel slices containing a genomic DNA restriction digest, is not to be considered an isolated nucleic acid.

“Lignin” refers to a polyphenolic polymeric substance of plant cells, with a complex, cross-linked, highly aromatic structure. Lignin is synthesized in plants principally from coniferyl alcohol (C₁₀H₁₂O₃) by extensive condensation polymerization. Lignin is the major noncarbohydrate structural constituent of plant cells in woody species.

“Modulation” of the level of biomass refers to the change in the level of the biomass that is observed as a result of expression of, or transcription from, an exogenous nucleic acid in a plant cell and/or plant. The change in level is measured relative to the corresponding level in control plants.

“NIR Model” refers to a series of validated mathematical equations that predict the chemical composition of a sample, based on NIR spectral data from the sample. The term also refers to a series of validated mathematical equations that predict saccharification conversion efficiency of a sample, based on NIR spectral data from the sample. In the case of saccharification conversion efficiency, a different NIR model is developed for each combination of pretreatment conditions and enzyme(s). NIR spectral data typically is obtained from the sample at a plurality of different wavelengths, and the mathematical equations are applied to the spectral data to calculate the predicted value. The calibration equations can be derived by regression among spectroscopic data for feedstock samples of the same type, e.g., by multiple-linear regression, by partial least squares, or by neural network analysis.

“Nucleic acid” and “polynucleotide” are used interchangeably herein, and refer to both RNA and DNA, including cDNA, genomic DNA, synthetic DNA, and DNA or RNA containing nucleic acid analogs. A nucleic acid can be double-stranded or single-stranded (i.e., a sense strand or an antisense strand). Non-limiting examples of polynucleotides include genes, gene fragments, exons, introns, messenger RNA (mRNA), transfer RNA, ribosomal RNA, siRNA, micro-RNA, ribozymes, cDNA, recombinant polynucleotides, branched polynucleotides, nucleic acid probes and nucleic acid primers. A polynucleotide may contain unconventional or modified nucleotides.

“Operably linked” refers to the positioning of a regulatory region and a sequence to be transcribed in a nucleic acid so that the regulatory region is effective for regulating transcription or translation of the sequence. For example, to operably link a coding sequence and a regulatory region, the translation initiation site of the translational reading frame of the coding sequence is typically positioned between one and about fifty nucleotides downstream of the regulatory region. A regulatory region can, however, be positioned as much as about 5,000 nucleotides upstream of the translation initiation site, or about 2,000 nucleotides upstream of the transcription start site.

“Pectin” refers to a polysaccharide having a backbone of α-(1-4)-linked D-galacturonic acid residues, with regions of 1,2-linked L-rhamnose. Side chains containing arabinose, xylose and galactose are present depending on the source material. Polygalacturonans, rhamnogalacturonans and some arabinans, galactans and arabinogalactans are pectins. Pectins are typically found in the middle lamella and primary wall of plant cells.

“Polypeptide” as used herein refers to a compound of two or more subunit amino acids, amino acid analogs, or other peptidomimetics, regardless of post-translational modification, e.g., phosphorylation or glycosylation. The subunits may be linked by peptide bonds or other bonds such as, for example, ester or ether bonds. Full-length polypeptides, truncated polypeptides, point mutants, insertion mutants, splice variants, chimeric proteins, and fragments thereof are encompassed by this definition.

“Progeny” includes descendants of a particular plant or plant line. Progeny of an instant plant include seeds formed on F₁, F₂, F₃, F₄, F₅, F₆ and subsequent generation plants, or seeds formed on BC₁, BC₂, BC₃, and subsequent generation plants, or seeds formed on F₁BC₁, F₁BC₂, F₁BC₃, and subsequent generation plants. The designation F₁ refers to the progeny of a cross between two parents that are genetically distinct. The designations F₂, F₃, F₄, F₅ and F₆ refer to subsequent generations of self- or sib-pollinated progeny of an F₁ plant.

“Regulatory region” refers to a nucleic acid having nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically comprises at least a core (basal) promoter. A regulatory region also may include at least one control element, such as an enhancer sequence, an upstream element or an upstream activation region (UAR). For example, a suitable enhancer is a cis-regulatory element (−212 to −154) from the upstream region of the octopine synthase (ocs) gene. Fromm et al., The Plant Cell, 1:977-984 (1989).

“Up-regulation” refers to regulation that increases the level of an expression product (mRNA, polypeptide, or both) relative to basal or native states.

“Vector” refers to a replicon, such as a plasmid, phage, or cosmid, into which another DNA segment may be inserted so as to bring about the replication of the inserted segment. Generally, a vector is capable of replication when associated with the proper control elements. The term “vector” includes cloning and expression vectors, as well as viral vectors and integrating vectors. An “expression vector” is a vector that includes a regulatory region.

### II. Polypeptides

Polypeptides described herein include biomass-modulating polypeptides. Biomass-modulating polypeptides can be effective to modulate biomass levels when expressed in a plant or plant cell. Such polypeptides typically contain at least one domain indicative of biomass-modulating polypeptides, as described in more detail herein. biomass-modulating polypeptides typically have an HMM bit score that is greater than 93 as described in more detail herein. In some embodiments, biomass-modulating polypeptides have greater than 80% identity to SEQ ID NOs: 2, 4, 5, 6, 8, 10, 12, 13, 15, 17, 18, 20, 22, 24, 25, 26, 27, 29, 30, 32, 34, 35, 37, 38, 39, 41, 43, 45, 47, 49, 51, 53, 55, 56, 57, 59, 60, 62, 63, 64, 65, 67, 69, 70, 71, 73, 74, 75, 77, 79, 80, 81, 82, 83, 84, 85, 86, 88, 90, 91, 92, 93, 94, 95, 96, 97, 98, 99, 100, 101, 102, 103, 104, 105, 106, 107, 108, 109, 110, 111, 112, 113, 115, 117, 119, 120, 121, 122, 123, 124, 126, 128, 129, 131, 133, 135, 137, 139, 140, 141, 142, 144, 146, 147, 148, 149, 150, 152, 154, 155, 157, 158, 160, 162, 163, 165, 167, 169, 171, 173, 175, 176, 178, 180, 182, 183, 185, 187, 189, 191, 193, 194, 195, 196, 197, 198, 199, 200, 201, 203, 205, 207, 208, 209, 211, 213, 214, 215, 217, 218, 219, 220, 222, 223, 224, 226, 228, 229, 231, 232, 233, 234, 235, 236, 237, 238, 239, 240, 242, 244, 245, 246, 247, 248, 249, 250, 251, 252, 254, 255, 256, 257, 258, 259, 260, 261, 262, 263, 264, 265, 266, 267, 268, 269, 270, 271, 272, 273, 274, 275, 276, 277, 278, 279, 281, 282, 284, 286, 287, 288, 289, 291, 293, 294, 295, 297, 299, 300, 302, 304, 306, 307, 308, 310, 311, 313, 315, 316, 317, 318, 319, 320, 322, 324, 326, 328, 330, 331, 333, 335, 337, 338, 340, 342, 343, 345, 346, 347, 349, 350, 351, 353, 354, 355, 357, 358, 360, 362, 363, 364, 365, 367, 369, 370, 371, 372, 373, 374, 375, 377, 378, 379, 380, 382, 383, 384, 385, 386, 387, 389, 390, 391, 393, 395, 396, 398, 400, 401, 403, 404, 406, 408, 409, 410, 411, 412, 414, 415, 417, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 456, 457, 459, 461, 462, 464, 466, 467, 468, 469, 470, 472, 474, 475, 477, 479, 480, 482, 483, 484, 485, 487, 488, 489, 490, 491, 492, 493, 494, 495, 497, 499, 501, 502, 503, 504, 506, 507, 509, 510, 511, 512, 514, 515, 516, 517, 518, 520, 522, 524, 526, 528, 530, 531, 532, 533, 534, 535, 536, 537, 538, 539, 540, 542, 543, 545, 546, 547, 548, 550, 551, 553, 554, 555, 557, 559, 560, 562, 563, 565, 566, 568, 570, 572, 573, 575, 576, 577, 579, 580, 581, 582, 583, 584, 585, 586, 587, 589, 590, 591, 592, 593, 595, 596, 597, 598, 599, 601, 602, 604, 605, 607, 609, 610, 611, 612, 613, 614, 615, 616, 617, 618, 619, 620, 621, 622, 623, 624, 625, 626, 627, 629, 631, 633, 634, 635, 36, 637, 638, 640, 642, 644, 646, 648, 650, 651, 653, 654, 655, 656, 657, 658, 659, 660, 662, 663, 664, 665, 666, 667, 669, 671, 672, 674, 676, 677, 679, 680, 682, 684, 685, 687, 689, 690, 691, 693, 694, 696, 697, 698, 00, 702, 704, 705, 706, 707, 709, 710, 712, 714, 715, 717, 718, 719, 720, 721, 722, 723, 725, 726, 727, 728, 730, 731, 732, 733, 734, 735, 737, 739, 741, 743, 744, 745, 746, 747, 749, 751, 752, 753, 755, 757, 759, 760, 762, 764, 766, 768, 770, 772, 773, 775, 776, 778, 780, 782, 784, 786, 788, 789, 791, 793, 794, 795, 796, 798, 800, 801, 802, 804, 805, 807, 809, 811, 813, 815, 817, 819, 820, 822, 823, 824, 825, 827, 828, 830, 832, 834, 836, 838, 840, 842, 843, 844, 846, 848, 849, 850, 851, 853, 855, 856, 857, 858, 860, 861, 863, 864, 866, 868, 870, 871, 873, 874, 876, 878, 880, 882, 883, 885, 887, 889, 890, 892, 893, 895, 896, 897, 899, 901, 902, 903, 905, 907, 908, 909, 910, 912, 914, 915, 916, 917, 919, 921, 923, 925, 926, 928, 930, 932, 934, 935, 936, 937, 939, 941, 943, 945, 947, 949, 950, 952, 953, 955, 957, 959, 960, 962, 964, 965, 967, 968, 970, 972, 974, 976, 978, 980, 981, 983, 985, 86, 87, 989, 991, 993, 995, 997, 998, 1000, 1002, 1003, 1005, 1007, 1008, 1009, 1011, 1013, 1014, 1016, 1018, 1020, 1022, 1023, 1024, 1025, 1026, 1027, 1029, 1031, 1033, 1034, 1036, 1038, 1039, 1041, 1042, 1043, 1044, 1045, 1046, 1048, 1049, 1050, 1051, 1052, 1054, 1056, 1058, 1060, 1061, 1062, 1064, 1066, 1067, 1069, 1070, 1071, 1072, 1074, 1076, 1077, 1078, 1079, 1081, 1083, 1085, 1087, 1088, 1090, 1092, 1093, 1095, 1096, 1097, 1099, 1101, 1102, 1104, 1105, 1107, 1108, 1110, 1111, 1112, 1113, 1114, 1115, 1117, 1119, 1120, 1121, 1122, 1124, 1126, 1128, 1130, 1132, 1133, 1135, 1137, 1139, 1141, 1142, 1144, 1146, 1148, 1150, 1152, 1154, 1156, 1157, 1158, 1160, 1162, 1164, 1166, 1168, 1169, 1171, 1173, 1175, 1176, 1177, 1179, 1180, 1181, 1183, 1185, 1187, 1188, 1190, 1192, 1193, 1194, 1196, 1198, 1200, 1201, 1203, 1205, 1207, 1209, 1210, 1212, 1214, 1216, 1218, 1219, 1221, 1222, 1224, 1226, 1228, 1230, 1232, 1234, 1235, 1236, 1238, 1240, 1241, 1243, 1245, 1247, 1249, 1251, 1252, 1253, 1255, 1257, 1259, 1261, 1263, 1265, 1266, 1268, 1270, 1272, 1274, 1276, 1278, 1279, 1281, 1283, 1285, 1287, 1289, 1291, 1293, 1294, 1296, 1297, 1299, 1300, 1302, 1304, 1306, 1308, 1309, 1310, 1312, 1313, 1314, 1316, 1318, 1320, 1322, 1324, 1325, 1326, 1328, 1329, 1330, 1332, 1333, 1334, 1336, 1338, 1339, 1340, 1341, 1342, 1343, 1344, 1345, 1347, 1349, 1350, 1352, 1353, 1355, 1357, 1358, 1359, 1360, 1361, 1363, 1364, 1366, 1367, 1369, 1370, 1371, 1372, 1373, 1375, 1376, 1377, 1379, 1380, 1381, 1382, 1384, 1386, 1388, 1389, 1391, 1393, 1395, 1397, 1399, 1401, 1402, 1403, 1405, 1407, 1409, 1410, 1412, 1414, 1416, 1418, 1420, 1421, 1422, 1424, 1426, 1428, 1429, 1431, 1433, 1435, 1437, 1439, 1440, 1441, 1443, 1445, 1446, 1448, 1450, 1452, 1454, 1456, 1457, 1459, 1461, 1463, 1465, 1467, 1469, 1471, 1473, 1475, 1477, 1479, 1481, 1482, 1483, 1485, 1486, 1488, 1489, 1490, 1492, 1493, 1495, 1497, 1498, 1500, 1502, 1503, 1505, 1506, 1507, 1508, 1509, 1511, 1512, 1514, 1515, 1516, 1518, 1520, 1522, 1523, 1524, 1525, 1526, 1528, 1529, 1531, 1533, 1534, 1535, 1537, 1538, 1540, 1542, 1544, 1546, 1547, 1549, 1550, 1552, 1554, 1555, 1557, 1558, 1560, 1562, 1563, 1564, 1565, 1568, 1569, 1571, 1572, 1574, 1575, 1577, 1578, 1580, 1581, 1583, 1584, 1586, 1588, 1589, 1591, 1593, 1594, 1595, 1596, 1598, 1599, 1600, 1602, 1603, 1604, 1605, 1607, 1609, 1611, 1612, 1613, 1615, 1616, 1618, 1619, 1621, 1622, 1624, 1626, 1628, 1629, 1630, 1632, 1634, 1636, 1637, 1639, 1640, 1642, 1644, 1646, 1647, 1648, 1649, 1650, 1652, 1654, 1656, 1657, 1658, 1660, 1661, 1662, 1664, 1666, 1668, 1669, 1670, 1671, 1672, 1674, 1676, 1677, 1679, 1680, 1681, 1682, 1683, 1684, 1686, 1688, 1689, 1691, 1693, 1695, 1697, 1699, 1700, 1701, 1702, 1704, 1705, 1706, 1707, 1708, 1710, 1711, 1712, 1713, 1714, 1715, 1717, 1719, 1721, 1723, 1724, 1725, 1727, 1729, 1731, 1732, 1734, 1735, 1737, 1739, 1741, 1743, 1745, 1746, 1747, 1749, 1751, 1753, 1755, 1757, 1758, 1759, 1761, 1762, 1764, 1765, 1766, 1767, 1769, 1770, 1772, 1773, 1774, 1776, 1777, 1779, 1781, 1783, 1784, 1786, 1788, 1790, 1792, 1794, 1795, 1797, 798, 1799, 1801, 1803, 1804, 1805, 1806, 1807, 1808, 1809, 1810, 1811, 1812, 1813, 1815, 1816, 1817, 1819, 1821, 1822, 1823, 1825, 1826, 1827, 1828, 1829, 1831, 1833, 1834, 1835, 1837, 1839, 1841, 1842, 1844, 1845, 1847, 1849, 1850, 1852, 1853, 1854, 1855, 1856, 1857, 1858, 1859, 1861, 1863, 1865, 1866, 1867, 1869, 1871, 1872, 1874, 1876, 1877, 1879, 1881, 1882, 1883, 1885, 1887, 1889, 1891, 1893, 1894, 1895, 1897, 1898, 1900, 1902, 1903, 1904, 1906, 1908, 1910, 1912, 1913, 1915, 1917, 1918, 1919, 1920, 1922, 1924, 1926, 1928, 1929, 1931, 1933, 1935, 1936, 1938, 1940, 1942, 1944, 1946, 1948, 1949, 1951, 1953, 1955, 1957, 1959, 1961, 1963, 1965, 1967, 1969, 1971, 1972, 1973, 1974, 1975, 1976, 1978, 1979, 1980, 1982, 1983, 1985, 1986, 1987, 1989, 1990, 1992, 1994, 1996, 1997, 1999, 2000, 2002, 2003, 2005, 2007, 2008, 2009, 2010, 2011, 2013, 2015, 2016, 2017, 2018, 2019, 2021, 2022, 2023, 2024, 2026, 2028, 2030, 2031, 2032, 2034, 2036, 2037, 2038, 2040, 2041, 2042, 2043, 2044, 2046, 2047, 2048, 2049, 2051, 2053, 2055, 2056, 2058, 2060, 2062, 2064, 2066, 2067, 2069, 2070, 2072, 2073, 2074, 2075, 2076, 2077, 2078, 2079, 2081, 2083, 2085, 2087, 2088, 2090, 2091, 2092, 2093, 2095, 2097, 2098, 2100, 2102, 2104, 2105, 2106, 2108, 2110, 2112, 2113, 2115, 2117, 2118, 2119, 2120, 2121, 2122, 2124, 2126, 2128, 2129, 2130, 2132, 2133, 2135, 2136, 2137, 2138, 2140, 2141, 2142, 2143, 2144, 2146, 2148, 2149, 2150, 2151, 2152, 2153, 2154, 2156, 2158, 2159, 2160, 2161, 2162, 2163, 2165, 2167, 2169, 2170, 2171, 2172, 2173, 2174, 2175, 2176, 2177, 2178, 2179, 2181, 2183, 2184, 2186, 2187, 2188, 2190, 2192, 2194, 2195, 2196, 2197, 2198, 2199, 2200, 2201, 2202, 2203, 2205, 2207, 2209, 2211, 2213, 2215, 2217, 2219, 2221, 2223, 2225, 2226, 2228, 2229, 2231, 2233, 2235, 2236, 2237, 2238, 2239, 2240, 2241, 2243, 2244, 2246, 2248, 2250, 2251, 2253, 2255, 2257, 2259, 2261, 2262, 2264, 2265, 2267, 2269, 2270, 2272, 2273, 2275, 2276, 2278, 2279, 2281, 2282, 2284, 2285, 2286, 2288, 2289, 2291, 2293, 2295, 2296, 2297, 2298, 2299, 2300, 2302, 2304, 2306, 2307, 2309, 2310, 2312, 2313, 2314, 2315, 2316, 2317, 2318, 2320, 2321, 2323, 2325, 2327, 2329, 2330, 2332, 2334, 2335, 2337, 2339, 2341, 2343, 2345, 2346, 2347, 2348, 2350, 2352, 2354, 2356, 2357, 2358, 2360, 2361, 2362, 2364, 2366, 2367, 2369, 2371, 2373, 2375, 2376, 2378, 2380, 2381, 2383, 2384, 2386, 2388, 2390, 2391, 2392, 2394, 2396, 2397, 2399, 2401, 2402, 2403, 2405, 2407, 2408, 2409, 2411, 2413, 2415, 2417, 2418, 2419, 2421, 2422, 2423, 2424, 2425, 2427, 2428, 2429, 2430, 2432, 2434, 2435, 2437, 2439, 2440, 2441, 2442, 2443, 2445, 2446, 2448, 2449, 2451, 2452, 2454, 2456, 2457, 2458, 2459, 2460, 2461, 2462, 2463, 2464, 2466, 2468, 2469, 2470, 2471, 2472, 2474, 2475, 2476, 2477, 2478, 2480, 2482, 2483, 2484, 2485, 2486, 2487, 2488, 2489, 2490, 2491, 2493, 2494, 2495, 2496, 2497, 2498, 2499, 2500, 2502, 2503, 2505, 2506, 2507, 2509, 2510, 2511, 2512, 2513, 2514, 2516, 2517, 2518, 2519, 2521, 2522, 2523, 2525, 2527, 2529, 2530, 2531, 2533, 2535, 2537, 2539, 2540, 2541, 2542, 2544, 2546, 2547, 2548, 2550, 2551, 2552, 2554, 2556, 2557, 2558, 2559, 2561, 2562, 2564, 2565, 2567, 2569, 2571, 2572, 2573, 2574, 2575, 2576, 2578, 2580, 2581, 2583, 2584, 2585, 2587, 2588, 2589, 2590, 2592, 2593, 2594, 2595, 2597, 2599, 2601, 2603, 2604, 2606, 2608, 2609, 2611, 2612, 2614, 2615, 2616, 2617, 2619, 2621, 2623, 2624, 2626, 2627, 2628, 2629, 2631, 2632, 2634, 2636, 2638, 2640, 2641, 2642, 2644, 2646, 2647, 2648, 2649, 2650, 2652, 2654, 2656, 2658, 2659, 2661, 2663, 2665, 2667, 2668, 2670, 2672, 2674, 2675, 2677, 2679, 2681, 2683, 2685, 2687, 2689, 2691, 2693, 2695, 2697, 2699, 2701, 2703, 2704, 2706, 2708, 2710, 2711, 2712, 2714, 2716, 2717, 2719, 2721, 2722, 2724, 2726, 2728, 2730, 2732, 2734, 2735, 2736, 2738, 2739, 2740, 2741, 2743, 2745, 2746, 2748, 2750, 2751, 2753, 2754, 2756, 2758, 2760, 2762, 2764, 2766, 2768, 2770, 2771, 2772, 2773, 2775, 2777, 2778, 2780, 2781, 2782, 2784, 2786, 2788, 2790, 2791, 2792, 2794, 2796, 2797, 2799, 2801, 2802, 2803, 2804, 2806, 2807, 2809, 2810, 2811, 2813, 2815, 2816, 2818, 2819, 2821, 2822, 2823, 2825, 2826, 2828, 2830, 2832, 2833, 2835, 2836, 2838, 2840, 2842, 2844, 2845, 2846, 2847, 2849, 2850, 2851, 2852, 2853, 2855, 2856, 2857, 2858, 2859, 2861, 2862, 2863, 2864, 2866, 2867, 2868, 2869, 2870, 2872, 2873, 2875, 2876, 2877, 2878, 2880, 2882, 2883, 2885, 2886, 2888, 2890, 2891, 2892, 2893, 2894, 2896, 2898, 2900, 2902, 2903, 2904, 2906, 2907, 2908, 2909, 2910, 2911, 2915, 2917, 2918, 2919, 2920, 2921, 2922, 2923, 2924, or 2926, as described in more detail herein.

A. Domains Indicative of Biomass-Modulating Polypeptides

A biomass-modulating polypeptide can contain a Myb-like DNA-binding domain, which is predicted to be characteristic of a biomass-modulating polypeptide. SEQ ID NO:2880 sets forth the amino acid sequence of an Arabidopsis thaliana clone identified herein as CeresAnnot:831967 (SEQ ID NO:2879), which is predicted to encode a polypeptide containing a Myb-like DNA-binding domain. For example, a biomass-modulating polypeptide can comprise a Myb-like DNA-binding domain having 60 percent or greater sequence identity to residues 14 to 61 and 67 to 112 of SEQ ID NO:2880. In some embodiments, a biomass-modulating polypeptide can comprise a Myb-like DNA-binding domain having 60 percent or greater sequence identity to the Myb-like DNA-binding domain of one or more of the polypeptides set forth in SEQ ID NOs: 2880, 2882, 2883, 2885, 2886, 2888, 2890, 2891, 2892, 2893, 2894, 2896, 2898, 2900, 2902, 2903, 2904, 2906, 2907, 2908, 2909, 2910, 2911, or 2913. The Myb-like DNA-binding domains of such sequences are set forth in the Sequence Listing.

The amino acid sequence of a Myb-like DNA-binding domain is also set forth in SEQ ID NO:1137, the amino acid sequence encoded by the Panicum virgatum clone identified herein as CeresClone:1775942 (SEQ ID NO:1136). This sequence is predicted to encode a polypeptide containing a Myb-like DNA-binding domain. For example, a biomass-modulating polypeptide can comprise a Myb-like DNA-binding domain having 60 percent or greater sequence identity to residues 10 to 59 of SEQ ID NO:1137. In some embodiments, a biomass-modulating polypeptide can comprise a Myb-like DNA-binding domain having 60 percent or greater sequence identity to the Myb-like DNA-binding domain of one or more of the polypeptides set forth in SEQ ID NOs: 1137, 1139, 1141, 1142, 1144, 1146, 1148, 1150, 1152, 1154, 1156, 1157, 1158, 1160, 1162, 1164, 1166, 1168, 1169, 1171, 1173, 1175, 1176, 1177, 1179, 1180, 1181, 1183, 1185, 1187, 1188, 1190, 1192, 1193, 1194, 1196, 1198, 1200, 1201, 1203, 1205, 1207, 1209, 1210, 1212, 1214, 1216, 1218, 1219, 1221, 1222, 1224, 1226, 1228, 1230, 1232, 1234, 1235, 1236, 1238, 1240, 1241, 1243, 1245, 1247, 1249, 1251, 1252, 1253, 1255, 1257, 1259, 1261, 1263, 1265, 1266, 1268, or 1270. The Myb-like DNA-binding domains of such sequences are set forth in the Sequence Listing.

The amino acid sequence of a Myb-like DNA-binding domain is also set forth in SEQ ID NO:2619, the amino acid sequence encoded by the Panicum virgatum clone identified herein as CeresClone:1797459 (SEQ ID NO:2618). This amino acid sequence is predicted to encode a polypeptide containing a Myb-like DNA-binding domain. For example, a biomass-modulating polypeptide can comprise a Myb-like DNA-binding domain having 60 percent or greater sequence identity to residues 223 to 274 of SEQ ID NO:2619. In some embodiments, a biomass-modulating polypeptide can comprise a Myb-like DNA-binding domain having 60 percent or greater sequence identity to the Myb-like DNA-binding domain of one or more of the polypeptides set forth in SEQ ID NOs: 2619, 2621, 2623, 2624, 2626, 2627, 2628, 2629, 2631, 2632, 2634, 2636, 2638, 2640, 2641, 2642, 2644, 2646, 2647, 2648, 2649, 2650, or 2652. The Myb-like DNA-binding domains of such sequences are set forth in the Sequence Listing. The Myb-like DNA-binding domain family contains the DNA binding domains from Myb proteins, as well as the SANT domain family.

A biomass-modulating polypeptide can contain a K-box Domain, which is predicted to be characteristic of a biomass-modulating polypeptide. SEQ ID NO:403 sets forth the amino acid sequence of an Arabidopsis thaliana clone, identified herein as CeresClone:5055 (SEQ ID NO:402) that is predicted to encode a polypeptide containing a K-box domain. For example, a biomass-modulating polypeptide can comprise a K-box domain having 60 percent or greater sequence identity to residues 75 to 176 of SEQ ID NO:403. In some embodiments, a biomass-modulating polypeptide can comprise a K-box domain having 60 percent or greater sequence identity to the K-box domain of one or more of the polypeptides set forth in SEQ ID NOs: 403, 404, 406, 408, 409, 410, 411, 412, 414, 415, 417, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 456, or 457. The K-box domains of such sequences are set forth in the Sequence Listing. The K-box is a possible coiled-coil structure commonly found associated with SRF-type transcription factors. Lupas et al. Science 252:1162-1164 (1991)

A biomass-modulating polypeptide can contain a SRF-type transcription factor (DNA binding and dimerisation domain) (SRF-TF) domain, which is predicted to be characteristic of a biomass-modulating polypeptide. SEQ ID NO:403 sets forth the amino acid sequence of an Arabidopsis thaliana clone, identified herein as CeresClone:5055 (SEQ ID NO:402) that is predicted to encode a polypeptide containing a SRF-TF domain. For example, a biomass-modulating polypeptide can comprise a SRF-TF domain having 60 percent or greater sequence identity to residues 9 to 59 of SEQ ID NO:403. In some embodiments, a biomass-modulating polypeptide can comprise a SRF-TF domain having 60 percent or greater sequence identity to the SRF-TF domain of one or more of the polypeptides set forth in SEQ ID NOs: 403, 404, 406, 408, 409, 410, 411, 412, 414, 415, 417, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 456, or 457. The SRF-TF domains of such sequences are set forth in the Sequence Listing. SRF-TF has a core domain of around 90 amino acids that is sufficient for the activities of DNA-binding, dimerisation, and interaction with accessory factors. Within the core is a DNA-binding region, designated the MADS box, that is highly similar to eukaryotic regulatory proteins, such as the Agamous and Deficiens families of plant homeotic proteins. Proteins belonging to the MADS family function as dimmers, each subunit of which contributes an amphipathic alpha helix to form the anti-parallel coiled-coil DNA-binding element. The MADS-box domain is commonly associated with a K-box region.

A biomass-modulating polypeptide can contain a bZIP transcription factor domain (bZIP_{—}1). SEQ ID NO:669 sets forth the amino acid sequence of an Arabidopsis thaliana clone, identified herein as CeresClone:14432 (SEQ ID NO:668), which is predicted to encode a polypeptide containing a bZIP domain. For example, a biomass-modulating polypeptide can comprise a bZIP_{—}1 transcription factor domain having 60 percent or greater sequence identity to residues 68 to 122 of SEQ ID NO:669. In some embodiments, a biomass-modulating polypeptide can comprise a bZIP_{—}1 transcription factor domain having 60 percent or greater sequence identity to the bZIP_{—}1 transcription factor domain of one or more of the polypeptides set forth in SEQ ID NOs: 669, 671, 672, 674, 676, 677, 679, 680, 682, 684, 685, 687, 689, 690, or 691. The bZIP_{—}1 transcription factor domains of such sequences are set forth in the Sequence Listing. The basic-leucine zipper (bZIP) transcription factors of eukaryotic cells are proteins that contain a basic region mediating sequence-specific DNA-binding followed by a leucine zipper region required for dimerization.

A biomass-modulating polypeptide can contain a basic region leucine zipper domain (bZIP_{—}2). The domain is also present in SEQ ID NO:669, which sets forth the amino acid sequence of an Arabidopsis thaliana clone, identified herein as CeresClone:14432 (SEQ ID NO:668), that is predicted to encode a polypeptide containing a bZIP_{—}2 basic region leucine zipper. For example, a biomass-modulating polypeptide can comprise a bZIP_{—}2 basic region leucine zipper domain having 60 percent or greater sequence identity to residues 68 to 122 of SEQ ID NO:669. In some embodiments, a biomass-modulating polypeptide can comprise a bZIP_{—}2 basic region leucine zipper domain having 60 percent or greater sequence identity to the bZIP_{—}2 basic region leucine zipper domain of one or more of the polypeptides set forth in SEQ ID NOs: 669, 671, 672, 674, 676, 677, 679, 680, 682, 684, 685, 687, 689, 690, or 691. The bZIP_{—}2 basic region leucine zipper domains of such sequences are set forth in the Sequence Listing. The bZIP_{—}1 and bZIP_{—}2 families are members of the bZIP superfamily of eukaryotic transcription factors that contain a basic region adjacent to a leucine zipper.

A biomass-modulating polypeptide can contain KNOX1 domain. SEQ ID NO:1797 sets forth the amino acid sequence of an Arabidopsis thaliana clone, identified herein as CeresClone:21240 (SEQ ID NO:1796), that is predicted to encode a polypeptide containing KNOX1 domain. For example, a biomass-modulating polypeptide can comprise a KNOX1 domain having 60 percent or greater sequence identity to residues 157 to 201 of SEQ ID NO:1797. In some embodiments, a biomass-modulating polypeptide can comprise a KNOX1 domain having 60 percent or greater sequence identity to the KNOX1 domain of one or more of the polypeptides set forth in SEQ ID NOs: 1797, 1798, 1799, 1801, 1803, 1804, 1805, 1806, 1807, 1808, 1809, 1810, 1811, 1812, 1813, 1815, 1816, 1817, 1819, 1821, 1822, 1823, 1825, 1826, 1827, 1828, 1829, 1831, 1833, 1834, 1835, 1837, 1839, 1841, 1842, 1844, 1845, 1847, 1849, 1850, 1852, 1853, 1854, 1855, 1856, 1857, 1858, 1859, 1861, 1863, 1865, 1866, 1867, 1869, 1871, 1872, 1874, 1876, 1877, 1879, 1881, 1882, 1883, or 1885. The KNOX1 domains of such sequences are set forth in the Sequence Listing. KNOX1 plays a role in suppressing target gene expression. Nagasaki et al., Plant Cell, 13: 2085-2098 (2001).

A biomass-modulating polypeptide can contain KNOX2 domain. SEQ ID NO:1797 sets forth the amino acid sequence of an Arabidopsis thaliana clone, identified herein as CeresClone:21240 (SEQ ID NO:1796), that is predicted to encode a polypeptide containing KNOX2 domain. For example, a biomass-modulating polypeptide can comprise a KNOX2 domain having 60 percent or greater sequence identity to residues 213 to 268 of SEQ ID NO:1797. In some embodiments, a biomass-modulating polypeptide can comprise a KNOX2 domain having 60 percent or greater sequence identity to the KNOX2 domain of one or more of the polypeptides set forth in SEQ ID NOs: 1797, 1798, 1799, 1801, 1803, 1804, 1805, 1806, 1807, 1808, 1809, 1810, 1811, 1812, 1813, 1815, 1816, 1817, 1819, 1821, 1822, 1823, 1825, 1826, 1827, 1828, 1829, 1831, 1833, 1834, 1835, 1837, 1839, 1841, 1842, 1844, 1845, 1847, 1849, 1850, 1852, 1853, 1854, 1855, 1856, 1857, 1858, 1859, 1861, 1863, 1865, 1866, 1867, 1869, 1871, 1872, 1874, 1876, 1877, 1879, 1881, 1882, 1883, or 1885. The KNOX2 domains of such sequences are set forth in the Sequence Listing. Together, the KNOX 2 and KNOX1 domains form the MEINOX region, in which the KNOX2 domain appears to be required for homodimerization.

A biomass-modulating polypeptide can contain a Fasciclin Myb-like DNA-binding domain, which is predicted to be characteristic of a biomass-modulating polypeptide. SEQ ID NO:1518 sets forth the amino acid sequence of a Panicum virgatum clone identified herein as CeresClone:1724055 (SEQ ID NO:1517), which is predicted to encode a polypeptide containing a Fasciclin domain. For example, a biomass-modulating polypeptide can comprise a Fasciclin domain having 60 percent or greater sequence identity to residues 202 to 334 of SEQ ID NO:1518. In some embodiments, a biomass-modulating polypeptide can comprise a Fasciclin domain having 60 percent or greater sequence identity to the Fasciclin domain of one or more of the polypeptides set forth in SEQ ID NOs: 1518, 1520, 1522, 1523, 1524, 1525, 1526, 1528, 1529, 1531, 1533, 1534, 1535, 1537, 1538, 1540, 1542, 1544, 1546, 1547, 1549, 1550, 1552, 1554, 1555, 1557, 1558, 1560, 1562, 1563, 1564, 1565, or 1568. The Fasciclin domains of such sequences are set forth in the Sequence Listing. Fasciclin-like arabinogalactan proteins (FLAs) are a subclass of arabinogalactan proteins (AGPs) that have predicted AGP-like glycosylated regions and putative cell adhesion domains known as fasciclin domains. The putative role of FLAs as cell adhesion molecules suggests that they are vital components of the interactions that occur in the plant extracellular matrix.

A biomass-modulating polypeptide can contain an O-methyltransferase domain (Methyltransf_{—}2), which is predicted to be characteristic of a biomass-modulating polypeptide. SEQ ID NO: 717 sets forth the amino acid sequence of an Triticum aestivum clone, identified herein as CeresClone:570057 (SEQ ID NO:716), that is predicted to encode a polypeptide containing an O-methyltransferase domain. For example, a biomass-modulating polypeptide can comprise an O-methyltransferase domain having 60 percent or greater sequence identity to residues 31 to 82 of SEQ ID NO:717. In some embodiments, a biomass-modulating polypeptide can comprise an O-methyltransferase domain having 60 percent or greater sequence identity to the O-methyltransferase domain of one or more of the polypeptides set forth in SEQ ID NOs: 717, 718, 719, 720, 721, 722, 723, 725, 726, 727, 728, 730, 731, 732, 733, 734, 735, 737, 739, 741, 743, 744, 745, 746, or 747. The O-methyltransferase domains of such sequences are set forth in the Sequence Listing. This domain includes a range of O-methyltransferases that utilize S-adenosyl methionine as a substrate. Methyltransferases catalyze methyl transfer from S-adenosyl-L-methionine (AdoMet) to either nitrogen, oxygen, or carbon atoms and modify DNA, RNA, proteins, and small molecules (e.g., catechol). O-methyltransferases have a common catalytic domain structure, which might be universal among S-adenosyl-L-methionine (AdoMet)-dependent methyltransferases. The O-methyltransferase family is a member of Methyltransferase superfamily.

A biomass-modulating polypeptide can contain a Dimerisation domain, which is predicted to be characteristic of a biomass-modulating polypeptide. SEQ ID NO:717 sets forth the amino acid sequence of an Triticum aestivum clone, identified herein as CeresClone:570057 (SEQ ID NO:716), that is predicted to encode a polypeptide containing an Dimerisation domain. For example, a biomass-modulating polypeptide can comprise an Dimerisation domain having 60 percent or greater sequence identity to residues 91 to 255 of SEQ ID NO:717. In some embodiments, a biomass-modulating polypeptide can comprise an Dimerisation domain having 60 percent or greater sequence identity to the Dimerisation domain of one or more of the polypeptides set forth in SEQ ID NOs: 717, 718, 719, 720, 721, 722, 723, 725, 726, 727, 728, 730, 731, 732, 733, 734, 735, 737, 739, 741, 743, 744, 745, 746, or 747. The Dimerisation domains of such sequences are set forth in the Sequence Listing. This domain is found at the N-terminus of a variety of plant O-methyltransferases. The Dimerisation domain family is a member of the Helix-turn-helix (HTH) superfamily, which comprises a diverse range of DNA-binding domains.

A biomass-modulating polypeptide can contain an AP2 domain, which is predicted to be characteristic of a biomass-modulating polypeptide. SEQ ID NO:693 sets forth the amino acid sequence of a Glycine max clone, identified herein as CeresClone:691319 (SEQ ID NO:692) that is predicted to encode a polypeptide containing an AP2 domain. For example, a biomass-modulating polypeptide can comprise an AP2 domain having 60 percent or greater sequence identity to residues 32 to 83 of SEQ ID NO:693. In some embodiments, a biomass-modulating polypeptide can comprise an AP2 domain having 60 percent or greater sequence identity to the AP2 domain of one or more of the polypeptides set forth in SEQ ID NOs: 693, 694, 696, 697, 698, 700, 702, 704, 705, 706, 707, 709, 710, 712, 714, or 715. The AP2 domains of such sequences are set forth in the Sequence Listing. AP2 domain amino acid residues can bind to DNA and are typically found in transcription factor proteins.

A biomass-modulating polypeptide can contain an NAD dependent epimerase/dehydratase family domain (Epimerase), which is predicted to be characteristic of a biomass-modulating polypeptide. SEQ ID NO:1624 sets forth the amino acid sequence of a Panicum virgatum clone, identified herein as CeresClone:1724569 (SEQ ID NO:1623) which is predicted to encode a polypeptide containing an Epimerase domain. For example, a biomass-modulating polypeptide can comprise an Epimerase domain having 60 percent or greater sequence identity to residues 21 to 253 of SEQ ID NO:1624. In some embodiments, a biomass-modulating polypeptide can comprise an Epimerase domain having 60 percent or greater sequence identity to the Epimerase domain of one or more of the polypeptides set forth in SEQ ID NOs: 1624, 1626, 1628, 1629, 1630, 1632, 1634, 1636, 1637, 1639, 1640, 1642, 1644, 1646, 1647, 1648, 1649, 1650, 1652, 1654, 1656, 1657, or 1658. The Epimerase domains of such sequences are set forth in the Sequence Listing.

The amino acid sequence of an Epimerase domain is also set forth in SEQ ID NO:1054, the amino acid sequence encoded by the Panicum virgatum clone identified herein as CeresClone:1788124 (SEQ ID NO:1053). This sequence is predicted to encode a polypeptide containing an Epimerase domain. For example, a biomass-modulating polypeptide can comprise an Epimerase domain having 60 percent or greater sequence identity to residues 20 to 290 of SEQ ID NO:1054. In some embodiments, a biomass-modulating polypeptide can comprise an Epimerase domain having 60 percent or greater sequence identity to the Epimerase domain of one or more of the polypeptides set forth in SEQ ID NOs: 1054, 1056, 1058, 1060, 1061, 1062, 1064, 1066, 1067, 1069, 1070, 1071, 1072, 1074, 1076, 1077, 1078, 1079, 1081, 1083, 1085, 1087, 1088, 1090, 1092, 1093, 1095, 1096, 1097, 1099, 1101, 1102, 1104, or 1105. The Epimerase domains of such sequences are set forth in the Sequence Listing.

The amino acid sequence of an Epimerase domain is also set forth in SEQ ID NO:2708, the amino acid sequence encoded by the Panicum virgatum clone identified herein as CeresClone:1788124 (SEQ ID NO:2707). This sequence is predicted to encode a polypeptide containing an Epimerase domain. For example, a biomass-modulating polypeptide can comprise an Epimerase domain having 60 percent or greater sequence identity to residues 10 to 253 of SEQ ID NO:2708. In some embodiments, a biomass-modulating polypeptide can comprise an Epimerase domain having 60 percent or greater sequence identity to the Epimerase domain of one or more of the polypeptides set forth in SEQ ID NOs: 2708, 2710, 2711, 2712, 2714, 2716, 2717, 2719, 2721, 2722, 2724, 2726, 2728, 2730, 2732, 2734, 2735, 2736, 2738, 2739, 2740, 2741, 2743, 2745, or 2746. The Epimerase domains of such sequences are set forth in the Sequence Listing.

The amino acid sequence of an Epimerase domain is also set forth in SEQ ID NO:2090, the amino acid sequence encoded by the Panicum virgatum clone identified herein as CeresClone:1788124 (SEQ ID NO:2089). This sequence is predicted to encode a polypeptide containing an Epimerase domain. For example, a biomass-modulating polypeptide can comprise an Epimerase domain having 60 percent or greater sequence identity to residues 79 to 342 of SEQ ID NO:2090. In some embodiments, a biomass-modulating polypeptide can comprise an Epimerase domain having 60 percent or greater sequence identity to the Epimerase domain of one or more of the polypeptides set forth in SEQ ID NOs: 2090, 2091, 2092, 2093, 2095, 2097, 2098, 2100, 2102, 2104, 2105, 2106, 2108, 2110, 2112, 2113, 2115, 2117, 2118, 2119, 2120, 2121, 2122, 2124, 2126, 2128, 2129, 2130, 2132, 2133, 2135, 2136, 2137, 2138, 2140, 2141, 2142, 2143, or 2144. The Epimerase domains of such sequences are set forth in the Sequence Listing.

The amino acid sequence of an Epimerase domain is also set forth in SEQ ID NO:2323, the amino acid sequence encoded by the Panicum virgatum clone identified herein as CeresClone:1821162 (SEQ ID NO:2322). This sequence is predicted to encode a polypeptide containing an Epimerase domain. For example, a biomass-modulating polypeptide can comprise an Epimerase domain having 60 percent or greater sequence identity to residues 14 to 258 of SEQ ID NO:2323. In some embodiments, a biomass-modulating polypeptide can comprise an Epimerase domain having 60 percent or greater sequence identity to the Epimerase domain of one or more of the polypeptides set forth in SEQ ID NOs: 2323, 2325, 2327, 2329, 2330, 2332, 2334, 2335, 2337, 2339, 2341, 2343, 2345, 2346, 2347, 2348, 2350, 2352, 2354, 2356, 2357, 2358, 2360, 2361, 2362, 2364, 2366, 2367, 2369, 2371, 2373, 2375, 2376, 2378, 2380, 2381, 2383, 2384, 2386, 2388, 2390, 2391, 2392, 2394, 2396, 2397, 2399, 2401, 2402, 2403, 2405, 2407, 2408, 2409, 2411, 2413, 2415, 2417, 2418, 2419, 2421, 2422, 2423, 2424, 2425, 2427, 2428, 2429, 2430, 2432, 2434, or 2435. The Epimerase domains of such sequences are set forth in the Sequence Listing.

The amino acid sequence of an Epimerase domain is also set forth in SEQ ID NO:2028, the amino acid sequence encoded by the Panicum virgatum clone identified herein as CeresClone:1830065 (SEQ ID NO:2027). This sequence is predicted to encode a polypeptide containing an Epimerase domain. For example, a biomass-modulating polypeptide can comprise an Epimerase domain having 60 percent or greater sequence identity to residues 13 to 258 of SEQ ID NO:2028. In some embodiments, a biomass-modulating polypeptide can comprise an Epimerase domain having 60 percent or greater sequence identity to the Epimerase domain of one or more of the polypeptides set forth in SEQ ID NOs: 2028, 2030, 2031, 2032, 2034, 2036, 2037, 2038, 2040, 2041, 2042, 2043, 2044, 2046, 2047, 2048, 2049, 2051, 2053, 2055, 2056, 2058, 2060, 2062, 2064, 2066, 2067, 2069, 2070, 2072, 2073, 2074, 2075, 2076, 2077, 2078, 2079, 2081, 2083, 2085, 2087, or 2088. NAD dependent epimerase/dehydratase family is a member of the FAD/NAD(P)-binding Rossmann fold superfamily, which is characterized by redox enzymes having two domains. One domain, termed the catalytic domain, confers substrate specificity and the precise reaction of the enzyme. The other domain, which is common to this class of redox enzymes, is a Rossmann-fold domain. The Rossmann domain binds nicotinamide adenine dinucleotide (NAD+) and it is this cofactor that reversibly accepts a hydride ion, which is lost or gained by the substrate in the redox reaction. Rossmann domains have an alpha/beta fold, which has a central beta sheet, with approximately five alpha helices found surrounding the beta sheet. The inter sheet crossover of the stands in the sheet form the NAD+ binding site. Bashton and Chothia, J Mol Biol, 315:927-939 (2002).

The amino acid sequence of an X8 domain is also set forth in SEQ ID NO:1887, the amino acid sequence encoded by the Panicum virgatum clone identified herein as 1724782 (SEQ ID NO:1886). This sequence is predicted to encode a polypeptide containing an X8 domain. For example, a biomass-modulating polypeptide can comprise an X8 domain having 60 percent or greater sequence identity to residues 21 to 106 of SEQ ID NO:1887. In some embodiments, a biomass-modulating polypeptide can comprise an X8 domain having 60 percent or greater sequence identity to X8 domain of one or more of the polypeptides set forth in SEQ ID NOs: 1887, 1889, 1891, 1893, 1894, 1895, 1897, 1898, 1900, 1902, 1903, 1904, 1906, 1908, 1910, 1912, 1913, 1915, 1917, 1918, 1919, 1920, 1922, 1924, 1926, 1928, 1929, 1931, 1933, 1935, 1936, 1938, 1940, 1942, or 1944. The X8 domains of such sequences are set forth in the Sequence Listing.

The amino acid sequence of an X8 domain is also set forth in SEQ ID NO:2746, the amino acid sequence encoded by the Panicum virgatum clone identified herein as CeresClone:1790340 (SEQ ID NO:2747). This amino acid is predicted to encode a polypeptide containing an X8 domain. For example, a biomass-modulating polypeptide can comprise an X8 domain having 60 percent or greater sequence identity to residues 368 to 452 of SEQ ID NO:2748. In some embodiments, a biomass-modulating polypeptide can comprise an X8 domain having 60 percent or greater sequence identity to X8 domain of one or more of the polypeptides set forth in SEQ ID NOs: 2748, 2750, 2751, 2753, 2754, 2756, 2758, 2760, 2762, 2764, 2766, 2768, 2770, 2771, 2772, 2773, 2775, 2777, 2778, 2780, 2781, 2782, 2784, 2786, 2788, 2790, 2791, 2792, 2794, 2796, 2797, 2799, 2801, 2802, 2803, 2804, 2806, 2807, 2809, 2810, 2811, 2813, 2815, 2816, 2818, 2819, 2821, 2822, 2823, 2825, 2826, 2828, 2830, 2832, 2833, 2835, 2836, 2838, 2840, 2842, 2844, 2845, 2846, 2847, 2849, 2850, 2851, 2852, 2853, 2855, 2856, 2857, 2858, 2859, 2861, 2862, 2863, 2864, 2866, 2867, 2868, 2869, 2870, 2872, 2873, 2875, 2876, 2877, or 2878. The X8 domains of such sequences are set forth in the Sequence Listing. The X8 domain contains 6 conserved cysteine residues that may form three disulphide bridges. The domain is found in an Olive pollen allergen and at the C-terminus of family 17 glycosyl hydrolases. Barral et al., J Immunol, 172: 3644-3651 (2004); Henrissat and Davies, Plant Physiol, 124:1515-1519 (2000). This domain may be involved in carbohydrate binding. Henrissat et al., Plant Mol Biol, 47: 55-72 (2001).

A biomass-modulating polypeptide can contain a Peroxidase domain, which is predicted to be characteristic of a biomass-modulating polypeptide. SEQ ID NO:1318 sets forth the amino acid sequence of a Panicum virgatum clone, identified herein as CeresClone:1725400 (SEQ ID NO:1317), that is predicted to encode a polypeptide containing a Peroxidase domain. For example, a biomass-modulating polypeptide can comprise a Peroxidase domain having 60 percent or greater sequence identity to residues 56 to 315 of SEQ ID NO:1318. In some embodiments, a biomass-modulating polypeptide can comprise a Peroxidase domain having 60 percent or greater sequence identity to the Peroxidase domain of one or more of the polypeptides set forth in SEQ ID NOs: 1318, 1320, 1322, 1324, 1325, 1326, 1328, 1329, 1330, 1332, 1333, 1334, 1336, 1338, 1339, 1340, 1341, 1342, 1343, 1344, 1345, 1347, 1349, 1350, 1352, 1353, 1355, 1357, 1358, 1359, 1360, 1361, 1363, 1364, 1366, 1367, 1369, 1370, 1371, 1372, 1373, 1375, 1376, 1377, 1379, 1380, 1381, 1382, 1384, 1386, 1388, 1389, 1391, 1393, 1395, 1397, 1399, 1401, 1402, 1403, 1405, 1407, 1409, 1410, 1412, 1414, 1416, 1418, 1420, 1421, 1422, or 1424. The Peroxidase domains of such sequences are set forth in the Sequence Listing. Peroxidases are heme-containing enzymes that use hydrogen peroxide as the electron acceptor to catalyse a number of oxidative reactions. Plant peroxidases have tissue-specific functions including removal of hydrogen peroxide from chloroplasts and cytosol, oxidation of toxic compounds, biosynthesis of the cell wall, defense responses towards wounding, indole-3-acetic acid (IAA) catabolism, and ethylene biosynthesis, for example. The heme group is embedded between the alpha domains of the Peroxidase domain shows a.

A biomass-modulating polypeptide can contain a DUF563 domain, which is predicted to be characteristic of a biomass-modulating polypeptide. SEQ ID NO:459 sets forth the amino acid sequence of a Panicum virgatum clone, identified herein as CeresClone:1726076 (SEQ ID NO:458), which is predicted to encode a polypeptide containing a DUF563 domain. For example, a biomass-modulating polypeptide can comprise a DUF563 domain having 60 percent or greater sequence identity to residues 35 to 257 of SEQ ID NO:459. In some embodiments, a biomass-modulating polypeptide can comprise a DUF563 domain having 60 percent or greater sequence identity to the DUF563 domain of one or more of the polypeptides set forth in SEQ ID NOs: 459, 461, 462, 464, 466, 467, 468, 469, 470, 472, 474, 475, 477, 479, 480, 482, 483, 484, 485, 487, 488, 489, 490, 491, 492, 493, 494, 495, 497, or 499. The DUF563 domains of such sequences are set forth in the Sequence Listing.

The amino acid sequence of a DUF563 domain is also set forth in SEQ ID NO:2746, the amino acid sequence encoded by the Panicum virgatum clone identified herein as CeresClone:1790340 (SEQ ID NO:2747). This amino acid is predicted to encode a polypeptide containing a DUF563 domain. For example, a biomass-modulating polypeptide can comprise a DUF563 domain having 60 percent or greater sequence identity to residues 109 to 357 of SEQ ID NO:846. In some embodiments, a biomass-modulating polypeptide can comprise a DUF563 domain having 60 percent or greater sequence identity to the DUF563 domain of one or more of the polypeptides set forth in SEQ ID NOs: 846, 848, 849, 850, 851, 853, 855, 856, 857, 858, 860, 861, 863, 864, 866, 868, 870, 871, 873, 874, or 876. The DUF563 domains of such sequences are set forth in the Sequence Listing.

The amino acid sequence of an DUF563 domain is also set forth in SEQ ID NO:1107, the amino acid sequence encoded by the Panicum virgatum clone identified herein as CeresClone:1781320 (SEQ ID NO:1106). This amino acid is predicted to encode a polypeptide containing an DUF563 domain. For example, a biomass-modulating polypeptide can comprise a DUF563 domain having 60 percent or greater sequence identity to residues 252 to 501 of SEQ ID NO:1107. In some embodiments, a biomass-modulating polypeptide can comprise a DUF563 domain having 60 percent or greater sequence identity to the DUF563 domain of one or more of the polypeptides set forth in SEQ ID NOs: 1107, 1108, 1110, 1111, 1112, 1113, 1114, 1115, 1117, 1119, 1120, 1121, 1122, 1124, 1126, 1128, 1130, 1132, 1133, or 1135. The DUF563 domains of such sequences are set forth in the Sequence Listing.

A biomass-modulating polypeptide can contain an UDP-glucoronosyl and UDP-glucosyl transferases domain (UDPGT), which is predicted to be characteristic of a biomass-modulating polypeptide. SEQ ID NO:335 sets forth the amino acid sequence of a Panicum virgatum clone, identified herein as CeresClone:1727475 (SEQ ID NO:334), which is predicted to encode a polypeptide containing an UDPGT domain. For example, a biomass-modulating polypeptide can comprise an UDPGT domain having 60 percent or greater sequence identity to residues 4 to 457 of SEQ ID NO: 335. In some embodiments, a biomass-modulating polypeptide can comprise an UDPGT domain having 60 percent or greater sequence identity to the UDPGT domain of one or more of the polypeptides set forth in SEQ ID NOs: 335, 337, 338, 340, 342, 343, 345, 346, 347, 349, 350, 351, 353, 354, 355, 357, 358, 360, 362, 363, 364, 365, 367, 369, 370, 371, 372, 373, 374, 375, 377, 378, 379, 380, 382, 383, 384, 385, 386, 387, 389, 390, 391, 393, 395, 396, 398, 400, or 401. The UDPGT domains of such sequences are set forth in the Sequence Listing.

The amino acid sequence of an UDPGT domain is also set forth in SEQ ID NO:1946, the amino acid sequence encoded by the Panicum virgatum clone identified herein as CeresClone:1788006 (SEQ ID NO:1945). This amino acid is predicted to encode a polypeptide containing an UDPGT domain. For example, a biomass-modulating polypeptide can comprise an UDPGT domain having 60 percent or greater sequence identity to residues 24 to 465 of SEQ ID NO:1946. In some embodiments, a biomass-modulating polypeptide can comprise an UDPGT domain having 60 percent or greater sequence identity to the UDPGT domain of one or more of the polypeptides set forth in SEQ ID NOs: 1946, 1948, 1949, 1951, 1953, 1955, 1957, 1959, 1961, 1963, 1965, 1967, 1969, 1971, 1972, 1973, 1974, 1975, 1976, 1978, 1979, 1980, 1982, 1983, 1985, 1986, 1987, 1989, 1990, 1992, 1994, 1996, 1997, 1999, 2000, 2002, 2003, 2005, 2007, 2008, 2009, 2010, 2011, 2013, 2015, 2016, 2017, 2018, 2019, 2021, 2022, 2023, 2024, or 2026. The UDPGT domains of such sequences are set forth in the Sequence Listing. UDP glycosyltransferases (UGT) are a superfamily of enzymes that catalyze the addition of the glycosyl group from a UTP-sugar to a small hydrophobic molecule. The UDPGT family includes flavonol O(3)-glucosyltransferase. These enzymes share a conserved domain of about 50 amino acid residues located in their C-terminal section. The UDPGT family is a member of the Glycosyl transferase clan, GT-B, which contains diverse glycosyltransferases that possess a Rossmann like fold. Liu and Mushegian, Protein Sci, 12: 1418-1431 (2003).

A biomass-modulating polypeptide can contain a Dirigent-like protein domain, which is predicted to be characteristic of a biomass-modulating polypeptide. SEQ ID NO:2525 sets forth the amino acid sequence of a Panicum virgatum clone, identified herein as CeresClone:1763298 (SEQ ID NO:2524), that is predicted to encode a polypeptide containing a Dirigent-like protein domain. For example, a biomass-modulating polypeptide can comprise a Dirigent-like protein domain having 60 percent or greater sequence identity to residues 1 to 146 of SEQ ID NO:2525. In some embodiments, a biomass-modulating polypeptide can comprise a Dirigent-like protein domain having 60 percent or greater sequence identity to the Dirigent-like protein domain of one or more of the polypeptides set forth in SEQ ID NOs: 2525, 2527, 2529, 2530, 2531, 2533, 2535, 2537, 2539, 2540, 2541, or 2542. The Dirigent-like protein domains of such sequences are set forth in the Sequence Listing. The Dirigent-like protein domain is conserved in the disease-resistance response family of proteins of Arabidopsis thaliana. The dirigent proteins are considered to mediate the free radical coupling of monolignol plant phenols in plants to yield lignans and lignins. Davin et al., Science, 275: 362-366 (1997); Burlat et al. Phytochemistry 57:883-8987 (2001).

A biomass-modulating polypeptide can contain a DUF1070 domain, which is predicted to be characteristic of a biomass-modulating polypeptide. SEQ ID NO:1272 sets forth the amino acid sequence of a Panicum virgatum clone, identified herein as CeresClone:1764605 (SEQ ID NO:1271), which is predicted to encode a polypeptide containing a DUF1070 domain. For example, a biomass-modulating polypeptide can comprise a DUF1070 domain having 60 percent or greater sequence identity to residues 35 to 257 of SEQ ID NO: 595. In some embodiments, a biomass-modulating polypeptide can comprise a DUF1070 domain having 60 percent or greater sequence identity to the DUF 1070 domain of one or more of the polypeptides set forth in SEQ ID NOs: 595, 597, 598, 600, 602, 603, 604, 605, 606, 608, 609, 610, 611, 613, 615, 616, 618, 619, 620, 622, 623, 625, 627, 629, 630, 632, 633, 634, 636, 637, 638, 639, 641, 642, or 643. The DUF1070 domains of such sequences are set forth in the Sequence Listing.

A biomass-modulating polypeptide can contain a Pectinesterase domain, which is predicted to be characteristic of a biomass-modulating polypeptide. SEQ ID NO:1660 sets forth the amino acid sequence of a Panicum virgatum clone, identified herein as CeresClone:1769660 (SEQ ID NO:1660), that is predicted to encode a polypeptide containing a Pectinesterase domain. For example, a biomass-modulating polypeptide can comprise a Pectinesterase domain having 60 percent or greater sequence identity to residues 35 to 257 of SEQ ID NO: 1660. In some embodiments, a biomass-modulating polypeptide can comprise a Pectinesterase domain having 60 percent or greater sequence identity to the Pectinesterase domain of one or more of the polypeptides set forth in SEQ ID NOs: 1660, 1661, 1662, 1664, 1666, 1668, 1669, 1670, 1671, 1672, 1674, 1676, 1677, 1679, 1680, 1681, 1682, 1683, 1684, 1686, 1688, 1689, 1691, 1693, 1695, 1697, 1699, 1700, 1701, 1702, 1704, 1705, 1706, 1707, 1708, 1710, 1711, 1712, 1713, 1714, 1715, 1717, 1719, 1721, 1723, 1724, or 1725. The Pectinesterase domains of such sequences are set forth in the Sequence Listing. Pectinesterase (pectin methylesterase) catalyses the de-esterification of pectin into pectate and methanol. The Pectinesterase family is a member of the Pec_lyase clan. Members of the Pec_lyase superfamily contain a right-handed beta helix as found in pectate lyase.

A biomass-modulating polypeptide can contain a Glycosyl hydrolase family 35 domain (Glyco_hydro_{—}35), which is predicted to be characteristic of a biomass-modulating polypeptide. SEQ ID NO: 2466 sets forth the amino acid sequence of a Panicum virgatum clone, identified herein as CeresClone:1777248 (SEQ ID NO:2465), that is predicted to encode a polypeptide containing a Glycosyl hydrolase family 35 domain. For example, a biomass-modulating polypeptide can comprise a Glycosyl hydrolase family 35 domain having 60 percent or greater sequence identity to residues 70 to 376 of SEQ ID NO: 2466. In some embodiments, a biomass-modulating polypeptide can comprise a Glycosyl hydrolase family 35 domain having 60 percent or greater sequence identity to the Glycosyl hydrolase family 35 domain of one or more of the polypeptides set forth in SEQ ID NOs: 2466, 2468, 2469, 2470, 2471, 2472, 2474, 2475, 2476, 2477, 2478, 2480, 2482, 2483, 2484, 2485, 2486, 2487, 2488, 2489, 2490, 2491, 2493, 2494, 2495, 2496, 2497, 2498, 2499, 2500, 2502, 2503, 2505, 2506, 2507, 2509, 2510, 2511, 2512, 2513, 2514, 2516, 2517, 2518, 2519, 2521, 2522, or 2523. The Glycosyl hydrolase family 35 domains of such sequences are set forth in the Sequence Listing. Glycosyl hydrolase family 35 comprises enzymes with beta-galactosidase activity. Beta-Galactosidase activity also increases in various fruits during ripening. The glycosyl hydrolase family 35 is a member of the Glyco_hydro_tim superfamily, which contains the contains a range of glycosyl hydrolase enzymes that possess a TIM barrel fold.

A biomass-modulating polypeptide can contain a Galactose binding lectin domain (Gal_Lectin), which is predicted to be characteristic of a biomass-modulating polypeptide. SEQ ID NO: 2466 sets forth the amino acid sequence of a Panicum virgatum clone, identified herein as CeresClone:1777248 (SEQ ID NO:2465), that is predicted to encode a polypeptide containing a Galactose binding lectin domain. For example, a biomass-modulating polypeptide can comprise a Galactose binding lectin domain having 60 percent or greater sequence identity to residues 830 to 906 of SEQ ID NO: 2466. In some embodiments, a biomass-modulating polypeptide can comprise a Galactose binding lectin domain having 60 percent or greater sequence identity to the Galactose binding lectin domain of one or more of the polypeptides set forth in SEQ ID NOs: 2466, 2468, 2469, 2470, 2471, 2472, 2474, 2475, 2476, 2477, 2478, 2480, 2482, 2483, 2484, 2485, 2486, 2487, 2488, 2489, 2490, 2491, 2493, 2494, 2495, 2496, 2497, 2498, 2499, 2500, 2502, 2503, 2505, 2506, 2507, 2509, 2510, 2511, 2512, 2513, 2514, 2516, 2517, 2518, 2519, 2521, 2522, or 2523. The Galactose binding lectin domains of such sequences are set forth in the Sequence Listing. The lectin binding domain of plant beta-galactosidases have been implicated in catabolism of cell wall sugars during fruit ripening.

A biomass-modulating polypeptide can contain an Exostosin family domain, which is predicted to be characteristic of a biomass-modulating polypeptide. SEQ ID NO:115 sets forth the amino acid sequence of a Panicum virgatum clone, identified herein as CeresClone:1788820 (SEQ ID NO:114), that is predicted to encode a polypeptide containing an Exostosin family domain. For example, a biomass-modulating polypeptide can comprise an Exostosin family domain having 60 percent or greater sequence identity to residues 54 to 358 of SEQ ID NO:115. In some embodiments, a biomass-modulating polypeptide can comprise an Exostosin family domain having 60 percent or greater sequence identity to the Exostosin family domain of one or more of the polypeptides set forth in SEQ ID NOs: 115, 117, 119, 120, 121, 122, 123, 124, 126, 128, 129, 131, 133, 135, 137, 139, 140, 141, 142, 144, 146, 147, 148, 149, 150, 152, 154, 155, 157, 158, 160, 162, 163, 165, 167, 169, 171, 173, 175, 176, 178, 180, 182, 183, 185, 187, 189, 191, 193, 194, 195, 196, 197, 198, 199, 200, 201, 203, 205, 207, 208, or 209. The Exostosin domains of such sequences are set forth in the Sequence Listing. The Exostosin family is based on the b-glucuronyltransferase domain of animal exostosins. In addition to the b-glucuronyltransferase domain, exostosins contain a 4-N-acetylglucosaminyltransferase domain. Many plant genes have been shown to encode proteins with significant sequence similarity to the animal exostosin b-glucuronyltransferase.

A biomass-modulating polypeptide can contain a RmlD substrate binding domain (RmlD_sub_bind), which is predicted to be characteristic of a biomass-modulating polypeptide. SEQ ID NO:2708 sets forth the amino acid sequence of a Panicum virgatum, identified herein as CeresClone:1789564 (SEQ ID NO:2707), that is predicted to encode a polypeptide containing a RmlD substrate binding domain. For example, a biomass-modulating polypeptide can comprise a RmlD substrate binding domain having 60 percent or greater sequence identity to residues 381 to 659 of SEQ ID NO:2708. In some embodiments, a biomass-modulating polypeptide can comprise a RmlD substrate binding domain having 60 percent or greater sequence identity to the RmlD substrate binding domain of one or more of the polypeptides set forth in SEQ ID NOs: 2708, 2710, 2711, 2712, 2714, 2716, 2717, 2719, 2721, 2722, 2724, 2726, 2728, 2730, 2732, 2734, 2735, 2736, 2738, 2739, 2740, 2741, 2743, 2745, or 2746. The RmlD substrate binding domains of such sequences are set forth in the Sequence Listing. The RmlD substrate binding domain is responsible for binding a sugar nucleotide. Blankenfeldt et al., Structure (Camb), 10: 773-786 (2002); Giraud et al., Nat Struct Biol, 7:398-402 (2000).

A biomass-modulating polypeptide can contain a Glycosyltransferase family 43 domain (Glyco_transf_{—}43), which is predicted to be characteristic of a biomass-modulating polypeptide. SEQ ID NO:1481 sets forth the amino acid sequence of a Panicum virgatum clone, identified herein as CeresClone:1789593 (SEQ ID NO:1480), that is predicted to encode a polypeptide containing a Glycosyltransferase family 43 domain. For example, a biomass-modulating polypeptide can comprise a Glycosyltransferase family 43 domain having 60 percent or greater sequence identity to residues 138 to 364 of SEQ ID NO:1481. In some embodiments, a biomass-modulating polypeptide can comprise a Glycosyltransferase family 43 domain having 60 percent or greater sequence identity to the Glycosyltransferase family 43 domain of one or more of the polypeptides set forth in SEQ ID NOs: 1481, 1482, 1483, 1485, 1486, 1488, 1489, 1490, 1492, 1493, 1495, 1497, 1498, 1500, 1502, 1503, 1505, 1506, 1507, 1508, 1509, 1511, 1512, 1514, 1515, or 1516. The Glycosyltransferase family 43 domains of such sequences are set forth in the Sequence Listing. The Glycosyltransferases are involved in biosynthesis of disaccharides, oligosaccharides, and polysaccharides. Glycosyl-transferases catalyse the transfer of sugar moieties from activated donor molecules to specific acceptor molecules, forming glycosidic bonds. Glycosyltransferase family 43 comprises enzymes with beta-glucuronyltransferase activity. Glycosyltransferase family 43 is a member of the GT-A clan, which contains diverse glycosyltransferases possessing a Rossmann-like fold. The Rossmann fold is a protein structural motif found in proteins that bind nucleotides, e.g., NAD.

A biomass-modulating polypeptide can contain a Glycosyl hydrolase family 17 domain (Glyco_hydro_{—}17), which is predicted to be characteristic of a biomass-modulating polypeptide. SEQ ID NO:2748 sets forth the amino acid sequence of a Panicum virgatum clone, identified herein as CeresClone:1790340 (SEQ ID NO:2747), that is predicted to encode a polypeptide containing a Glycosyl hydrolase family 17 domain. For example, a biomass-modulating polypeptide can comprise a Glycosyl hydrolase family 17 domain having 60 percent or greater sequence identity to residues 30 to 348 of SEQ ID NO: 2748. In some embodiments, a biomass-modulating polypeptide can comprise a Glycosyl hydrolase family 17 domain having 60 percent or greater sequence identity to the Glycosyl hydrolase family 17 domain of one or more of the polypeptides set forth in SEQ ID NOs: 2748, 2750, 2751, 2753, 2754, 2756, 2758, 2760, 2762, 2764, 2766, 2768, 2770, 2771, 2772, 2773, 2775, 2777, 2778, 2780, 2781, 2782, 2784, 2786, 2788, 2790, 2791, 2792, 2794, 2796, 2797, 2799, 2801, 2802, 2803, 2804, 2806, 2807, 2809, 2810, 2811, 2813, 2815, 2816, 2818, 2819, 2821, 2822, 2823, 2825, 2826, 2828, 2830, 2832, 2833, 2835, 2836, 2838, 2840, 2842, 2844, 2845, 2846, 2847, 2849, 2850, 2851, 2852, 2853, 2855, 2856, 2857, 2858, 2859, 2861, 2862, 2863, 2864, 2866, 2867, 2868, 2869, 2870, 2872, 2873, 2875, 2876, 2877, or 2878. The Glycosyl hydrolase family 17 domains of such sequences are set forth in the Sequence Listing. Glycoside hydrolase family 17 comprises enzymes such as endo-1,3-beta-glucosidase, lichenase, and exo-1,3-glucanase. The glycosyl hydrolase family is a member of the Glyco_hydro_tim superfamily, which contains a range of glycosyl hydrolase enzymes possessing a TIM barrel fold.

A biomass-modulating polypeptide can contain a Transferase family domain, which is predicted to be characteristic of a biomass-modulating polypeptide. SEQ ID NO:947 sets forth the amino acid sequence of a Panicum virgatum clone, identified herein as CeresClone:1790811 (SEQ ID NO:946), which is predicted to encode a polypeptide containing a Transferase family domain. For example, a biomass-modulating polypeptide can comprise a Transferase family domain having 60 percent or greater sequence identity to residues 5 to 439 of SEQ ID NO:947. In some embodiments, a biomass-modulating polypeptide can comprise a Transferase family domain having 60 percent or greater sequence identity to the Transferase family domain of one or more of the polypeptides set forth in SEQ ID NOs: 947, 949, 950, 952, 953, 955, 957, 959, 960, 962, 964, 965, 967, 968, 970, 972, 974, 976, 978, 980, 981, 983, 985, 986, 987, 989, 991, 993, 995, 997, 998, 1000, 1002, 1003, 1005, 1007, 1008, 1009, 1011, 1013, 1014, 1016, 1018, 1020, 1022, 1023, 1024, 1025, 1026, 1027, 1029, 1031, 1033, 1034, 1036, 1038, 1039, 1041, 1042, 1043, 1044, 1045, 1046, 1048, 1049, 1050, 1051, or 1052. The Transferase family domains of such sequences are set forth in the Sequence Listing.

The amino acid sequence of an Transferase family domain is also set forth in SEQ ID NO:1569, the amino acid sequence encoded by the Panicum virgatum clone identified herein as CeresClone:1900097 (SEQ ID NO:1568). This amino acid is predicted to encode a polypeptide containing an Transferase family domain. For example, a biomass-modulating polypeptide can comprise a Transferase family domain having 60 percent or greater sequence identity to residues 4 to 436 of SEQ ID NO:1569. In some embodiments, a biomass-modulating polypeptide can comprise a Transferase family domain having 60 percent or greater sequence identity to the Transferase family domain of one or more of the polypeptides set forth in SEQ ID NOs: 1569, 1571, 1572, 1574, 1575, 1577, 1578, 1580, 1581, 1583, 1584, 1586, 1588, 1589, 1591, 1593, 1594, 1595, 1596, 1598, 1599, 1600, 1602, 1603, 1604, 1605, 1607, 1609, 1611, 1612, 1613, 1615, 1616, 1618, 1619, 1621, or 1622. The Transferase family domains of such sequences are set forth in the Sequence Listing. This family includes enzymes such as anthranilate N-hydroxycinnamoyl/benzoyltransferase and deacetylvindoline 4-O-acetyltransferase EC:2.3.1.107. The transferase family is a member of the CoA-dependent acyltransferase superfamily. All characterised families in this clan are involved in CoA-dependent acyltransferase and have a characteristic HXXXD motif.

A biomass-modulating polypeptide can contain an alpha/beta hydrolase fold domain (Abhydrolase_{—}3), which is predicted to be characteristic of a biomass-modulating polypeptide. SEQ ID NO:1727 sets forth the amino acid sequence of an Arabidopsis clone, identified herein as CeresClone:1792586 (SEQ ID NO:1726), which is predicted to encode a polypeptide containing an alpha/beta hydrolase fold domain. For example, a biomass-modulating polypeptide can comprise an alpha/beta hydrolase fold domain having 60 percent or greater sequence identity to residues 85 to 310 of SEQ ID NO:1727. In some embodiments, a biomass-modulating polypeptide can comprise an alpha/beta hydrolase fold domain having 60 percent or greater sequence identity to the alpha/beta hydrolase fold domain of one or more of the polypeptides set forth in SEQ ID NOs: 1727, 1729, 1731, 1732, 1734, 1735, 1737, 1739, 1741, 1743, 1745, 1746, 1747, 1749, 1751, 1753, 1755, 1757, 1758, 1759, 1761, 1762, 1764, 1765, 1766, 1767, 1769, 1770, 1772, 1773, 1774, 1776, 1777, 1779, 1781, 1783, 1784, 1786, 1788, 1790, 1792, 1794, or 1795. The alpha/beta hydrolase fold domains of such sequences are set forth in the Sequence Listing. The alpha/beta hydrolase fold is common to a number of hydrolytic enzymes of widely differing phylogenetic origin and catalytic function. The core of each enzyme is an alpha/beta-sheet (rather than a barrel), containing 8 strands connected by helices. The enzymes are believed to have diverged from a common ancestor, preserving the arrangement of the catalytic residues. All have a catalytic triad, the elements of which are borne on loops, which are the best conserved structural features of the fold.

A biomass-modulating polypeptide can contain a Chitinase Class I domain (Glycohydro_{—}19), which is predicted to be characteristic of a biomass-modulating polypeptide. SEQ ID NO:211 sets forth the amino acid sequence of a Panicum virgatum clone, identified herein as CeresClone:1801750 (SEQ ID NO:210), that is predicted to encode a polypeptide containing a Chitinase Class I domain. For example, a biomass-modulating polypeptide can comprise a Chitinase Class I domain having 60 percent or greater sequence identity to residues 29 to 246 of SEQ ID NO:211. In some embodiments, a biomass-modulating polypeptide can comprise a Chitinase Class I domain having 60 percent or greater sequence identity to the Chitinase Class I domain of one or more of the polypeptides set forth in SEQ ID NOs: 211, 213, 214, 215, 217, 218, 219, 220, 222, 223, 224, 226, 228, 229, 231, 232, 233, 234, 235, 236, 237, 238, 239, 240, 242, 244, 245, 246, 247, 248, 249, 250, 251, 252, 254, 255, 256, 257, 258, 259, 260, 261, 262, 263, 264, 265, 266, 267, 268, 269, 270, 271, 272, 273, 274, 275, 276, 277, 278, 279, 281, or 282. The Chitinase Class I domains of such sequences are set forth in the Sequence Listing. Chitinases are enzymes that catalyze the hydrolysis of the beta-1,4-N-acetyl-D-glucosamine linkages in chitin polymers. Chitinases belong to glycoside hydrolase families 18 or 19. Henrissat, Biochem J. 280:309-16 (1991). Chitinases of family 19 (also known as classes IA or I and IB or II) are enzymes from plants that function in the defense against fungal and insect pathogens. Class IA/I and IB/II enzymes differ in the presence (IA/I) or absence (IB/II) of a N-terminal chitin-binding domain. The catalytic domain of these enzymes consist of about 220 to 230 amino acid residues.

A biomass-modulating polypeptide can contain a Pollen allergen domain (Pollen_allerg_{—}1), which is predicted to be characteristic of a biomass-modulating polypeptide. SEQ ID NO:629 sets forth the amino acid sequence of a Panicum virgatum clone, identified herein as CeresClone:1804242 (SEQ ID NO:628), that is predicted to encode a polypeptide containing a Pollen allergen domain. For example, a biomass-modulating polypeptide can comprise a Pollen allergen domain having 60 percent or greater sequence identity to residues 164 to 241 of SEQ ID NO:629. In some embodiments, a biomass-modulating polypeptide can comprise a Pollen allergen domain having 60 percent or greater sequence identity to the Pollen allergen domain of one or more of the polypeptides set forth in SEQ ID NOs: 629, 631, 633, 634, 635, 636, 637, 638, 640, 642, 644, 646, 648, 650, 651, 653, 654, 655, 656, 657, 658, 659, 660, 662, 663, 664, 665, 666, or 667. The Pollen allergen domains of such sequences are set forth in the Sequence Listing. This family contains Lolium perenne allergens Lol PI, PII, and PIII. Ansari et al. Biochemistry, 28: 8665-8670 (1989). Expansins share a high degree of sequence similarity with the Lol PI family of allergens. Expansins mediate cell wall extension in plants. Expansins promote sustained slippage between wall polymers without hydrolytic activity.

A biomass-modulating polypeptide can contain a rare lipoprotein A (RlpA)-like double-psi beta barrel domain (DPBB_{—}1), which is predicted to be characteristic of a biomass-modulating polypeptide. SEQ ID NO:629 sets forth the amino acid sequence of a Panicum virgatum clone, identified herein as CeresClone:1804242 (SEQ ID NO:628), that is predicted to encode a polypeptide containing a rare lipoprotein A (RlpA)-like double-psi beta barrel domain. For example, a biomass-modulating polypeptide can comprise a rare lipoprotein A (RlpA)-like double-psi beta barrel domain having 60 percent or greater sequence identity to residues 67 to 153 of SEQ ID NO:629. In some embodiments, a biomass-modulating polypeptide can comprise a rare lipoprotein A (RlpA)-like double-psi beta barrel domain having 60 percent or greater sequence identity to the rare lipoprotein A (RlpA)-like double-psi beta barrel domain of one or more of the polypeptides set forth in SEQ ID NOs: 629, 631, 633, 634, 635, 636, 637, 638, 640, 642, 644, 646, 648, 650, 651, 653, 654, 655, 656, 657, 658, 659, 660, 662, 663, 664, 665, 666, or 667. The rare lipoprotein A (RlpA)-like double-psi beta barrel domains of such sequences are set forth in the Sequence Listing. Rare lipoprotein A (RlpA) contains a conserved region that has the double-psi beta-barrel (DPBB) fold. This domain is found in the N terminus of pollen allergen. Bass et al., J Bacteriol, 178: 1154-1161 (1996).

A biomass-modulating polypeptide can contain a Raffinose synthase or seed inhibition (Sip 1) protein domain (Raffinose_syn), which is predicted to be characteristic of a biomass-modulating polypeptide. SEQ ID NO:2544 sets forth the amino acid sequence of a Panicum virgatum clone, identified herein as CeresClone:1806608 (SEQ ID NO:2543), that is predicted to encode a polypeptide containing a Raffinose synthase or seed inhibition (Sip 1) protein domain. For example, a biomass-modulating polypeptide can comprise a Raffinose synthase or seed inhibition (Sip 1) protein domain having 60 percent or greater sequence identity to residues 7 to 752 of SEQ ID NO:2544. In some embodiments, a biomass-modulating polypeptide can comprise a Raffinose synthase or seed inhibition (Sip 1) protein domain having 60 percent or greater sequence identity to the Raffinose synthase or seed inhibition (Sip 1) protein domain of one or more of the polypeptides set forth in SEQ ID NOs: 2544, 2546, 2547, 2548, 2550, 2551, 2552, 2554, 2556, 2557, 2558, 2559, 2561, 2562, 2564, 2565, 2567, 2569, 2571, 2572, 2573, 2574, 2575, 2576, 2578, 2580, 2581, 2583, 2584, 2585, 2587, 2588, 2589, 2590, 2592, 2593, 2594, 2595, or 2597. The Raffinose synthase or seed inhibition (Sip 1) protein domains of such sequences are set forth in the Sequence Listing. This family consists of several raffinose synthase proteins, also known as seed inhibition (Sip1) proteins. Raffinose synthase (EC 2.4.1.82) channels sucrose into the raffinose oligosaccharide pathway. Peterbauer et al., Planta, 215: 839-846 (2002).

A biomass-modulating polypeptide can contain a 4-alpha-glucanotransferase domain (Glyco_hydro_{—}77), which is predicted to be characteristic of a biomass-modulating polypeptide. SEQ ID NO:2437 sets forth the amino acid sequence of a Panicum virgatum clone, identified herein as CeresClone:1815770 (SEQ ID NO:2436), that is predicted to encode a polypeptide containing a 4-alpha-glucanotransferase domain. For example, a biomass-modulating polypeptide can comprise a 4-alpha-glucanotransferase domain having 60 percent or greater sequence identity to residues 90 to 579 of SEQ ID NO: 2437. In some embodiments, a biomass-modulating polypeptide can comprise 4-alpha-glucanotransferase domain having 60 percent or greater sequence identity to the 4-alpha-glucanotransferase domain of one or more of the polypeptides set forth in SEQ ID NOs: 2437, 2439, 2440, 2441, 2442, 2443, 2445, 2446, 2448, 2449, 2451, 2452, 2454, 2456, 2457, 2458, 2459, 2460, 2461, 2462, 2463, or 2464. The 4-alpha-glucanotransferase domains of such sequences are set forth in the Sequence Listing. 4-alpha-glucanotransferase belongs to the glycoside hydrolase family 77 and transfers a segment of a (1,4)-alpha-D-glucan to a new 4-position in an acceptor, which may be glucose or (1,4)-alpha-D-glucan. 4-alpha-glucantoransferase polypeptides may have a role in starch metabolism. Takaha et al. Bichem J, 268(2);1391-1396 (1993)

A biomass-modulating polypeptide can contain a No Apical Meristem (NAM) protein domain, which is predicted to be characteristic of a biomass-modulating polypeptide. SEQ ID NO:2 sets forth the amino acid sequence of a Panicum virgatum clone, identified herein as CeresClone:1824159 (SEQ ID NO:1), which is predicted to encode a polypeptide containing a NAM protein domain. For example, a biomass-modulating polypeptide can comprise a NAM protein domain having 60 percent or greater sequence identity to residues 12 to 154 of SEQ ID NO:2. In some embodiments, a biomass-modulating polypeptide can comprise a NAM protein domain having 60 percent or greater sequence identity to the NAM protein domain of one or more of the polypeptides set forth in SEQ ID NOs: 2, 4, 5, 6, 8, 10, 12, 13, 15, 17, 18, 20, 22, 24, 25, 26, 27, 29, 30, 32, 34, 35, 37, 38, 39, 41, 43, 45, 47, 49, 51, 53, 55, 56, 57, 59, 60, 62, 63, 64, 65, 67, 69, 70, 71, 73, 74, 75, or 77. The NAM domains of such sequences are set forth in the Sequence Listing.

The amino acid sequence of a NAM protein domain is also set forth in SEQ ID NO:284, the amino acid sequence encoded by the Panicum virgatum clone identified herein as CeresClone:2014404 (SEQ ID NO:283). This amino acid is predicted to encode a polypeptide containing an NAM protein domain. For example, a biomass-modulating polypeptide can comprise a NAM protein domain having 60 percent or greater sequence identity to residues 7 to 125 of SEQ ID NO:284. In some embodiments, a biomass-modulating polypeptide can comprise a NAM protein domain having 60 percent or greater sequence identity to the NAM protein domain of one or more of the polypeptides set forth in SEQ ID NOs: 284, 286, 287, 288, 289, 291, 293, 294, 295, 297, 299, 300, 302, 304, 306, 307, or 308. The NAM domains of such sequences are set forth in the Sequence Listing. Polypeptides containing a NAM protein domain are associated with plant development. For example, mutations in the NAM protein of petunia result in the failure to develop a shoot apical meristem in petunia embryos. Sablowski and Meyerowitz, Cell, 92:93-103 (1998). Proteins containing a NAM protein domain may have a role in the transition between growth by cell division and cell expansion in stamens and petals. Souer, et al., Cell, 85:159-170 (1996).

A biomass-modulating polypeptide can contain a Glycosyl hydrolase family 18 domain (Glyco_hydro_{—}18), which is predicted to be characteristic of a biomass-modulating polypeptide. SEQ ID NO:501 sets forth the amino acid sequence of a Panicum virgatum clone, identified herein as CeresClone:1902646 (SEQ ID NO:500), that is predicted to encode a polypeptide containing a Glycosyl hydrolase family 18 domain. For example, a biomass-modulating polypeptide can comprise a Glycosyl hydrolase family 18 domain having 60 percent or greater sequence identity to residues 26 to 290 of SEQ ID NO:501. In some embodiments, a biomass-modulating polypeptide can comprise a Glycosyl hydrolase family 18 domain having 60 percent or greater sequence identity to the Glycosyl hydrolase family 18 domain of one or more of the polypeptides set forth in SEQ ID NOs: 501, 502, 503, 504, 506, 507, 509, 510, 511, 512, 514, 515, 516, 517, 518, 520, 522, 524, 526, 528, 530, 531, 532, 533, 534, 535, 536, 537, 538, 539, 540, 542, 543, 545, 546, 547, 548, 550, 551, 553, 554, 555, 557, 559, 560, 562, 563, 565, 566, 568, 570, 572, 573, 575, 576, 577, 579, 580, 581, 582, 583, 584, 585, 586, 587, 589, 590, 591, 592, 593, 595, 596, 597, 598, 599, 601, 602, 604, 605, 607, 609, 610, 611, 612, 613, 614, 615, 616, 617, 618, 619, 620, 621, 622, 623, 624, 625, 626, or 627. The Glycosyl hydrolase family 18 domains of such sequences are set forth in the Sequence Listing. Some members of this family belong to the chitinase class II group, which hydrolyse chitin oligosaccharides. The glycosyl hydrolase family 18 is a member of the Glyco_hydro_tim superfamily, which contains the contains a range of glycosyl hydrolase enzymes that possess a TIM barrel fold.

A biomass-modulating polypeptide can contain a Glycosyl hydrolase family 16 domain (Glyco_hydro_{—}16), which is predicted to be characteristic of a biomass-modulating polypeptide. SEQ ID NO:2190 sets forth the amino acid sequence of a Panicum virgatum clone, identified herein as CeresClone:1955550 (SEQ ID NO:2189), that is predicted to encode a polypeptide containing a Glycosyl hydrolase family 16 domain. For example, a biomass-modulating polypeptide can comprise a Glycosyl hydrolase family 16 domain having 60 percent or greater sequence identity to residues 23 to 204 of SEQ ID NO:2190. In some embodiments, a biomass-modulating polypeptide can comprise a Glycosyl hydrolase family 16 domain having 60 percent or greater sequence identity to the Glycosyl hydrolase family 16 domain of one or more of the polypeptides set forth in SEQ ID NOs: 2189, 2191, 2193, 2204, 2206, 2208, 2210, 2212, 2214, 2216, 2218, 2220, 2222, 2224, 2227, 2230, 2232, 2234, 2242, 2245, 2247, 2249, 2252, 2254, 2256, 2258, 2260, 2263, 2266, 2268, 2271, 2274, 2277, 2280, 2283, 2287, 2290, 2292, 2294, 2301, 2303, 2305, 2308, 2311, or 2319. The Glycosyl hydrolases family 16 domains of such sequences are set forth in the Sequence Listing. Glycoside hydrolase family 16 comprises enzymes such as lichenase, xyloglucan xyloglucosyltransferase, agarase, kappa-carrageenase, endo-beta-1,3-glucanase, endo-beta-1,3-1,4-glucanase, and endo-beta-galactosidase. The Glycosyl hydrolase family 16 is a member of the Concanavalin superfamily, which includes a diverse range of carbohydrate binding domains and glycosyl hydrolase enzymes that share a common structure.

A biomass-modulating polypeptide can contain a Xyloglycan endo-transglycosylase (XET_C) domain, which is predicted to be characteristic of a biomass-modulating polypeptide. SEQ ID NO:2190 sets forth the amino acid sequence of a Panicum virgatum clone, identified herein as CeresClone:1955550 (SEQ ID NO:2189), that is predicted to encode a polypeptide containing a Xyloglycan endo-transglycosylase domain. For example, a biomass-modulating polypeptide can comprise a Xyloglycan endo-transglycosylase domain having 60 percent or greater sequence identity to residues 228 to 280 of SEQ ID NO:2190. In some embodiments, a biomass-modulating polypeptide can comprise a Xyloglycan endo-transglycosylase domain having 60 percent or greater sequence identity to the Xyloglycan endo-transglycosylase domain of one or more of the polypeptides set forth in SEQ ID NOs: 2190, 2192, 2194, 2195, 2196, 2197, 2198, 2199, 2200, 2201, 2202, 2203, 2205, 2207, 2209, 2211, 2213, 2215, 2217, 2219, 2221, 2223, 2225, 2226, 2228, 2229, 2231, 2233, 2235, 2236, 2237, 2238, 2239, 2240, 2241, 2243, 2244, 2246, 2248, 2250, 2251, 2253, 2255, 2257, 2259, 2261, 2262, 2264, 2265, 2267, 2269, 2270, 2272, 2273, 2275, 2276, 2278, 2279, 2281, 2282, 2284, 2285, 2286, 2288, 2289, 2291, 2293, 2295, 2296, 2297, 2298, 2299, 2300, 2302, 2304, 2306, 2307, 2309, 2310, 2312, 2313, 2314, 2315, 2316, 2317, 2318, 2320, or 2321. The Xyloglycan endo-transglycosylase domains of such sequences are set forth in the Sequence Listing. The Xyloglycan endo-transglycosylase domain represents the C-terminus (approximately 60 residues) of plant xyloglucan endo-transglycosylase (XET). Xyloglucan is the predominant hemicellulose in the cell walls of most dicotyledons. With cellulose, it forms a network that strengthens the cell wall. XET catalyses the splitting of xyloglucan chains and the linking of the newly generated reducing end to the non-reducing end of another xyloglucan chain, thereby loosening the cell wall. Schroder et al. Planta, 204:242-251 (1998).

A biomass-modulating polypeptide can contain a Potato inhibitor I family domain, which is predicted to be characteristic of a biomass-modulating polypeptide. SEQ ID NO: 749 sets forth the amino acid sequence of a Panicum virgatum clone, identified herein as CeresClone:1955766 (SEQ ID NO:748), that is predicted to encode a polypeptide containing a Potato inhibitor I family domain. For example, a biomass-modulating polypeptide can comprise a Potato inhibitor I family domain having 60 percent or greater sequence identity to residues 17 to 76 of SEQ ID NO:749. In some embodiments, a biomass-modulating polypeptide can comprise a Potato inhibitor I family domain having 60 percent or greater sequence identity to the Potato inhibitor I family domain of one or more of the polypeptides set forth in SEQ ID NOs: 749, 751, 752, 753, 755, 757, 759, 760, 762, 764, 766, 768, 770, 772, 773, 775, 776, 778, 780, 782, 784, 786, 788, 789, 791, 793, 794, 795, 796, 798, 800, 801, 802, 804, 805, 807, 809, 811, 813, 815, 817, 819, 820, 822, 823, 824, 825, 827, 828, 830, or 832. The Potato inhibitor I family domains of such sequences are set forth in the Sequence Listing. Potato inhibitor type I sequences are found in various plant species. Peptide proteinase inhibitors can be single domain proteins or single or multiple domains within proteins (simple or compound inhibitors, respectively). In many cases, they are synthesized as part of a larger precursor protein, either as a prepropeptide or as an N-terminal domain associated with an inactive peptidase or zymogen.

A biomass-modulating polypeptide can contain a Plant Thionin domain (thionin), which is predicted to be characteristic of a biomass-modulating polypeptide.

SEQ ID NO:79 sets forth the amino acid sequence of a Panicum virgatum clone, identified herein as CeresClone:2010191 (SEQ ID NO:78), that is predicted to encode a polypeptide containing a Plant Thionin domain. For example, a biomass-modulating polypeptide can comprise a Plant Thionin domain having 60 percent or greater sequence identity to residues 25 to 67 of SEQ ID NO:79. In some embodiments, a biomass-modulating polypeptide can comprise a Plant Thionin domain having 60 percent or greater sequence identity to the Plant Thionin domain of one or more of the polypeptides set forth in SEQ ID NOs 79, 80, 81, 82, 83, 84, 85, 86, 88, 90, 91, 92, 93, 94, 95, 96, 97, 98, 99, 100, 101, 102, 103, 104, 105, 106, 107, 108, 109, 110, 111, 112, or 113. The Plant Thionin domains of such sequences are set forth in the Sequence Listing. Thionins are small, basic plant proteins, 45 to 50 amino acids in length, which include three or four conserved disulphide linkages. The proteins are toxic to animal cells, presumably attacking the cell membrane and rendering it permeable. This activity results in the inhibition of sugar uptake and allows potassium and phosphate ions, proteins, and nucleotides to leak from cells. Thionins are mainly found in seeds, where they may act as a defense against herbivory or microbial infections.

A biomass-modulating polypeptide can contain a Heavy-Metal-Associated (HMA) domain, which is predicted to be characteristic of a biomass-modulating polypeptide. SEQ ID NO:2915 sets forth the amino acid sequence of a Panicum virgatum clone, identified herein as CeresClone:2010191 (SEQ ID NO:2916), that is predicted to encode a polypeptide containing a HMA domain. For example, a biomass-modulating polypeptide can comprise a HMA domain having 60 percent or greater sequence identity to residues 6 to 73 of SEQ ID NO:2915. In some embodiments, a biomass-modulating polypeptide can comprise a HMA domain having 60 percent or greater sequence identity to the HMA domain of one or more of the polypeptides set forth in SEQ ID NOs: 2915, 2917, 2918, 2919, 2920, 2921, 2922, 2923, 2924, or 2926. The HMA domains of such sequences are set forth in the Sequence Listing. The HMA domains contain two conserved cysteines that are involved in metal binding. Proteins containing HMA domains may be involved in metal ion transport.

In some embodiments, a biomass-modulating polypeptide is truncated at the amino- or carboxy-terminal end of a naturally occurring polypeptide. A truncated polypeptide may retain certain domains of the naturally occurring polypeptide while lacking others. Thus, length variants that are up to 5 amino acids shorter or longer typically exhibit the biomass-modulating activity of a truncated polypeptide. In some embodiments, a truncated polypeptide is a dominant negative polypeptide. Expression in a plant of such a truncated polypeptide confers a difference in the level of biomass of a plant as compared to the corresponding level of a control plant that does not comprise the truncation.

B. Functional Homologs Identified by Reciprocal BLAST

In some embodiments, one or more functional homologs of a reference biomass-modulating polypeptide defined by one or more of the Pfam descriptions indicated above are suitable for use as biomass-modulating polypeptides. A functional homolog is a polypeptide that has sequence similarity to a reference polypeptide, and that carries out one or more of the biochemical or physiological function(s) of the reference polypeptide. A functional homolog and the reference polypeptide may be natural occurring polypeptides, and the sequence similarity may be due to convergent or divergent evolutionary events. As such, functional homologs are sometimes designated in the literature as homologs, or orthologs, or paralogs. Variants of a naturally occurring functional homolog, such as polypeptides encoded by mutants of a wild type coding sequence, may themselves be functional homologs. Functional homologs can also be created via site-directed mutagenesis of the coding sequence for a biomass-modulating polypeptide, or by combining domains from the coding sequences for different naturally-occurring biomass-modulating polypeptides (“domain swapping”). The term “functional homolog” is sometimes applied to the nucleic acid that encodes a functionally homologous polypeptide.

Functional homologs can be identified by analysis of nucleotide and polypeptide sequence alignments. For example, performing a query on a database of nucleotide or polypeptide sequences can identify homologs of biomass-modulating polypeptides. Sequence analysis can involve BLAST, Reciprocal BLAST, or PSI-BLAST analysis of nonredundant databases using a biomass-modulating polypeptide amino acid sequence as the reference sequence Amino acid sequence is, in some instances, deduced from the nucleotide sequence. Those polypeptides in the database that have greater than 40% sequence identity are candidates for further evaluation for suitability as a biomass-modulating polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains present in biomass-modulating polypeptides, e.g., conserved functional domains.

Conserved regions can be identified by locating a region within the primary amino acid sequence of a biomass-modulating polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains on the World Wide Web at sanger.ac.uk/Software/Pfam/ and pfam.janelia.org/. A description of the information included at the Pfam database is described in Sonnhammer et al., Nucl. Acids Res., 26:320-322 (1998); Sonnhammer et al., Proteins, 28:405-420 (1997); and Bateman et al., Nucl. Acids Res., 27:260-262 (1999). Conserved regions also can be determined by aligning sequences of the same or related polypeptides from closely related species. Closely related species preferably are from the same family. In some embodiments, alignment of sequences from two different species is adequate.

Typically, polypeptides that exhibit at least about 40% amino acid sequence identity are useful to identify conserved regions. Conserved regions of related polypeptides exhibit at least 45% amino acid sequence identity (e.g., at least 50%, at least 60%, at least 70%, at least 80%, or at least 90% amino acid sequence identity). In some embodiments, a conserved region exhibits at least 92%, 94%, 96%, 98%, or 99% amino acid sequence identity.

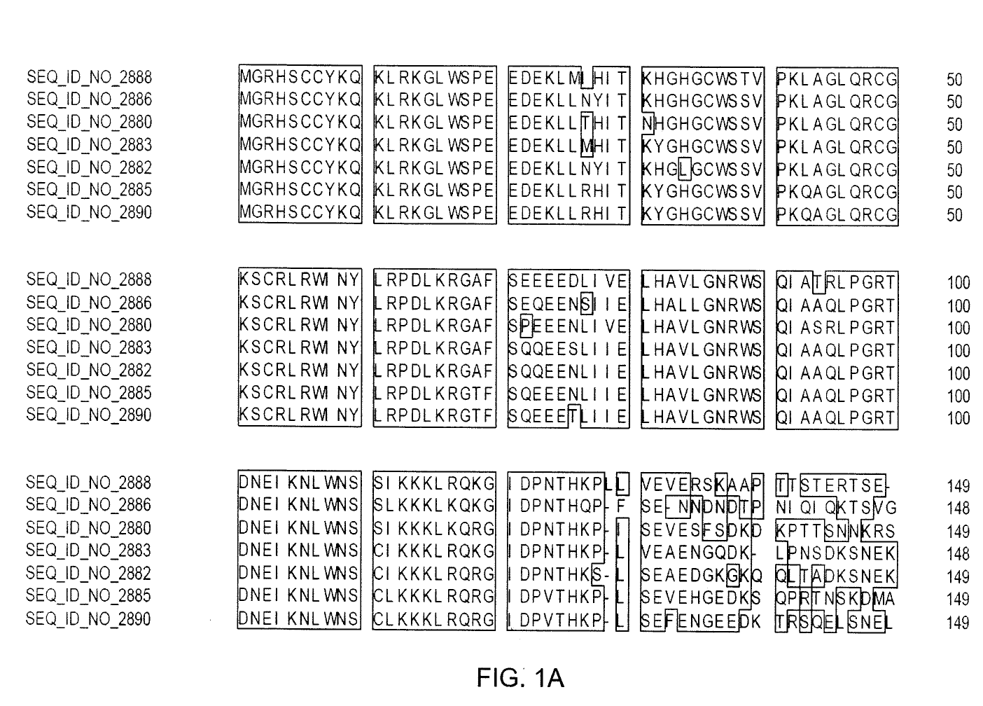

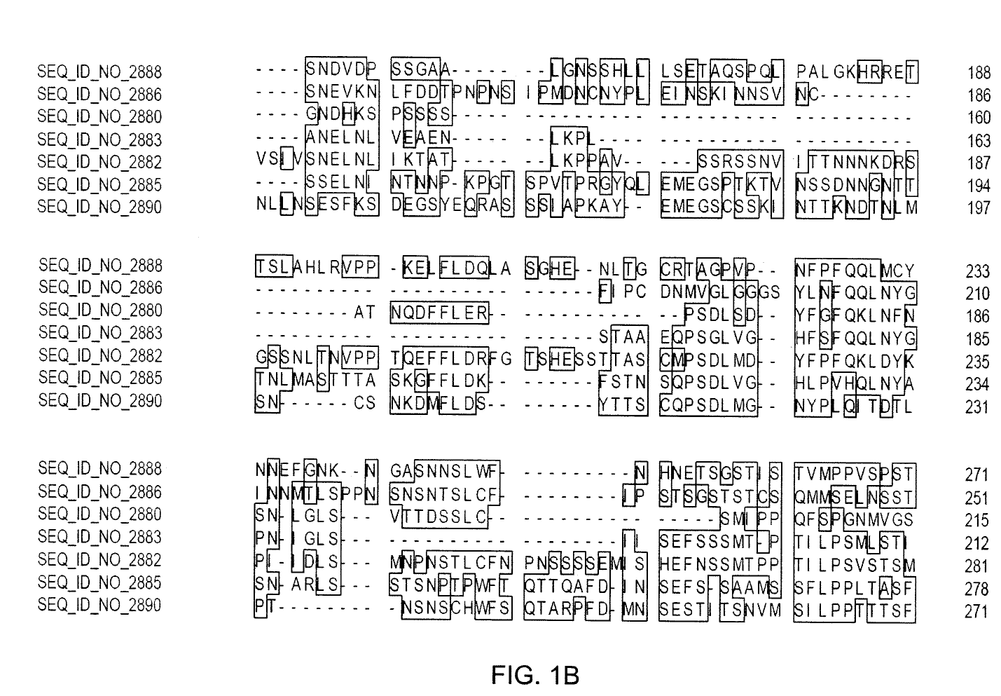

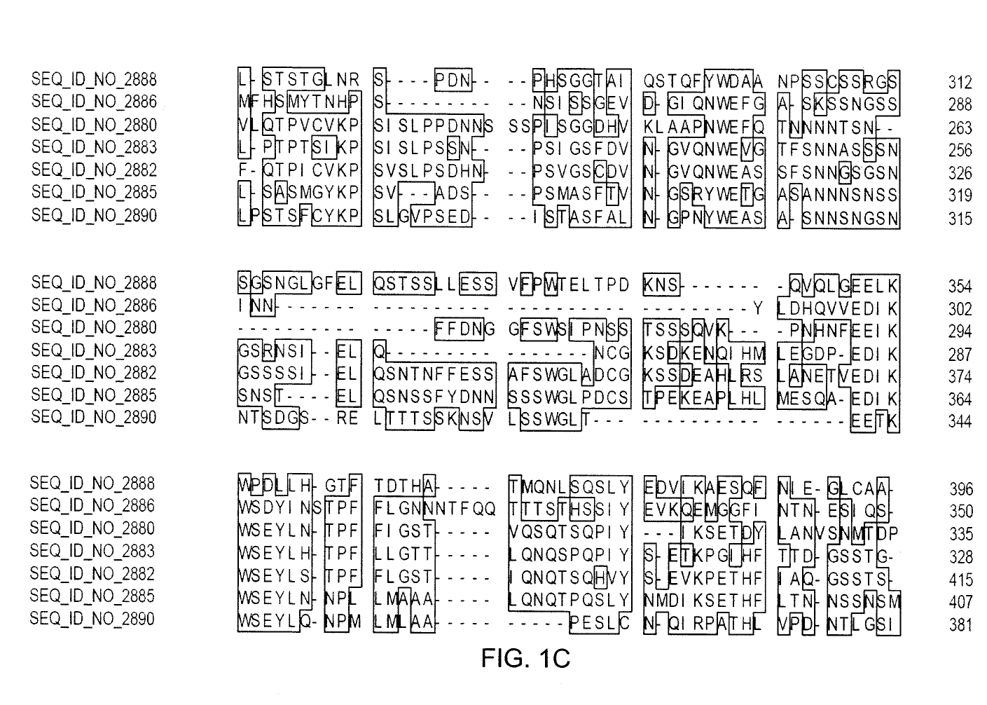

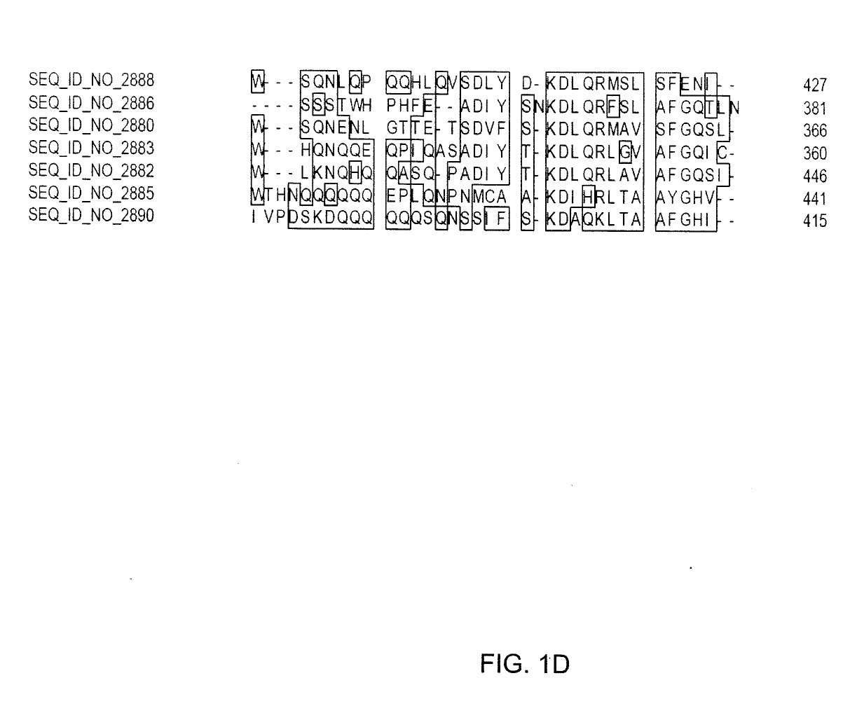

Examples of amino acid sequences of functional homologs of the polypeptide set forth in SEQ ID NO:2880 are provided in FIG. 1 and in the Sequence Listing. Such functional homologs include, for example, CeresAnnot:1469074 (SEQ ID NO:2882), GI:157346953 (SEQ ID NO:2883), CeresClone:1948185 (SEQ ID NO:2885), GI:87241324 (SEQ ID NO:2886), CeresClone:1398728 (SEQ ID NO:2888), CeresClone:513273 (SEQ ID NO:2890), GI:15223612 (SEQ ID NO:2891), GI:147833906 (SEQ ID NO:2892), GI:147767556 (SEQ ID NO:2893), GI:225437752 (SEQ ID NO:2894), CeresAnnot:1471467 (SEQ ID NO:2896), CeresAnnot:1444604 (SEQ ID NO:2898), CeresClone:1855571 (SEQ ID NO:2900), CeresClone:1929622 (SEQ ID NO:2902), GI:212722316 (SEQ ID NO:2903), GI:115461995 (SEQ ID NO:2904), CeresClone:1769439 (SEQ ID NO:2906), GI:115436030 (SEQ ID NO:2907), GI:125525446 (SEQ ID NO:2908), GI:56605400 (SEQ ID NO:2909), GI:28628949 (SEQ ID NO:2910), GI:194698774 (SEQ ID NO:2911), or CeresAnnot:845710 (SEQ ID NO:2913). In some cases, a functional homolog of SEQ ID NO:2880 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2880.

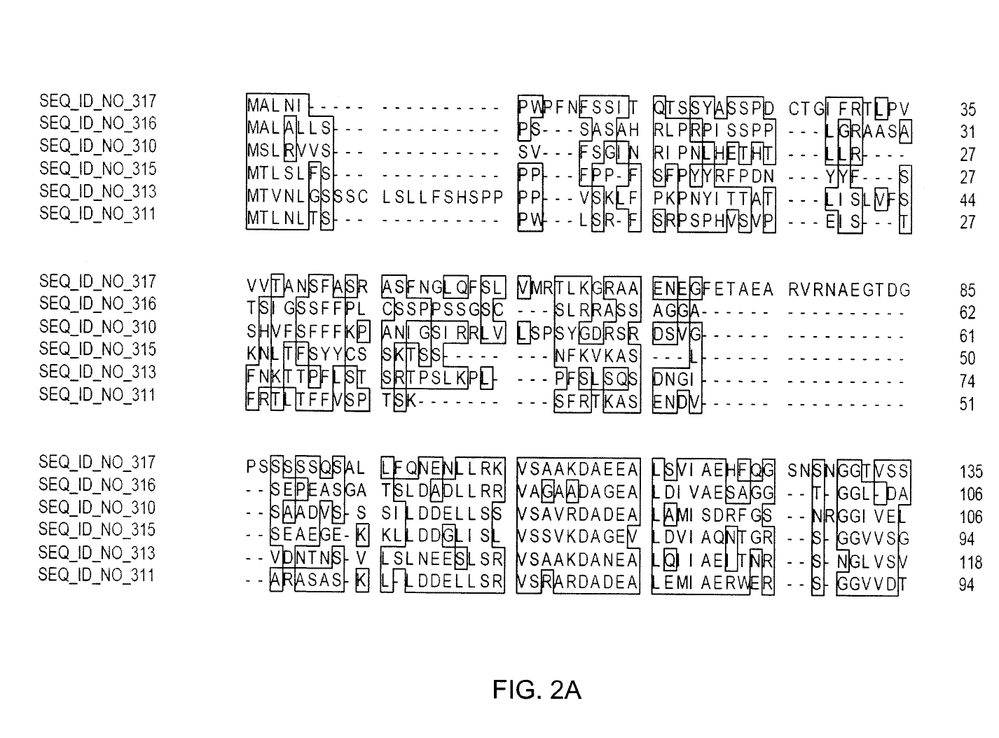

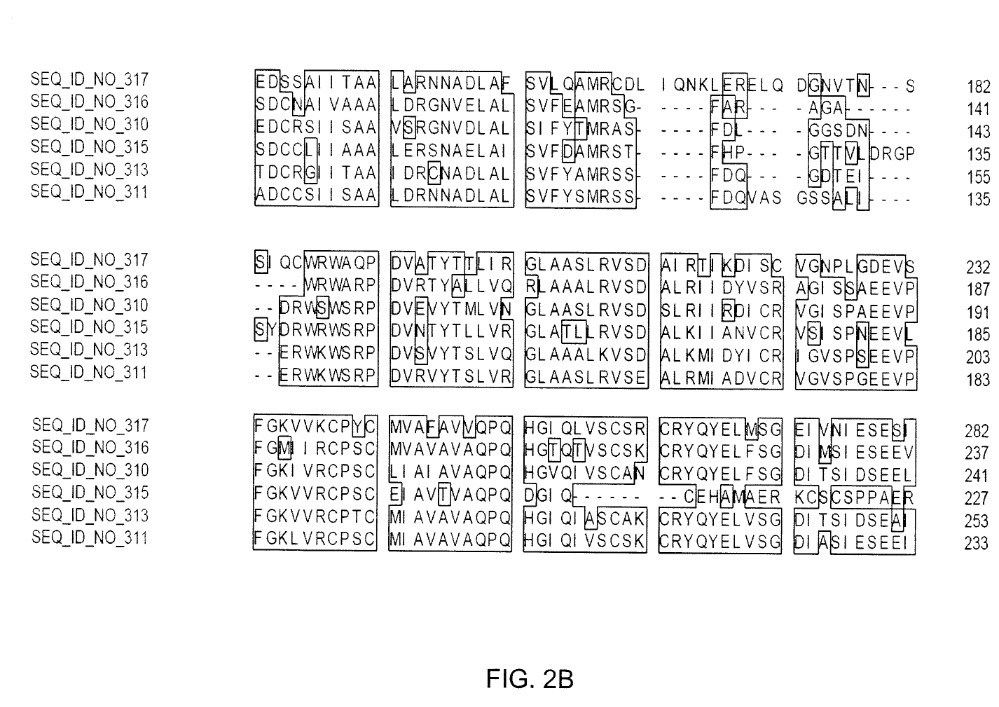

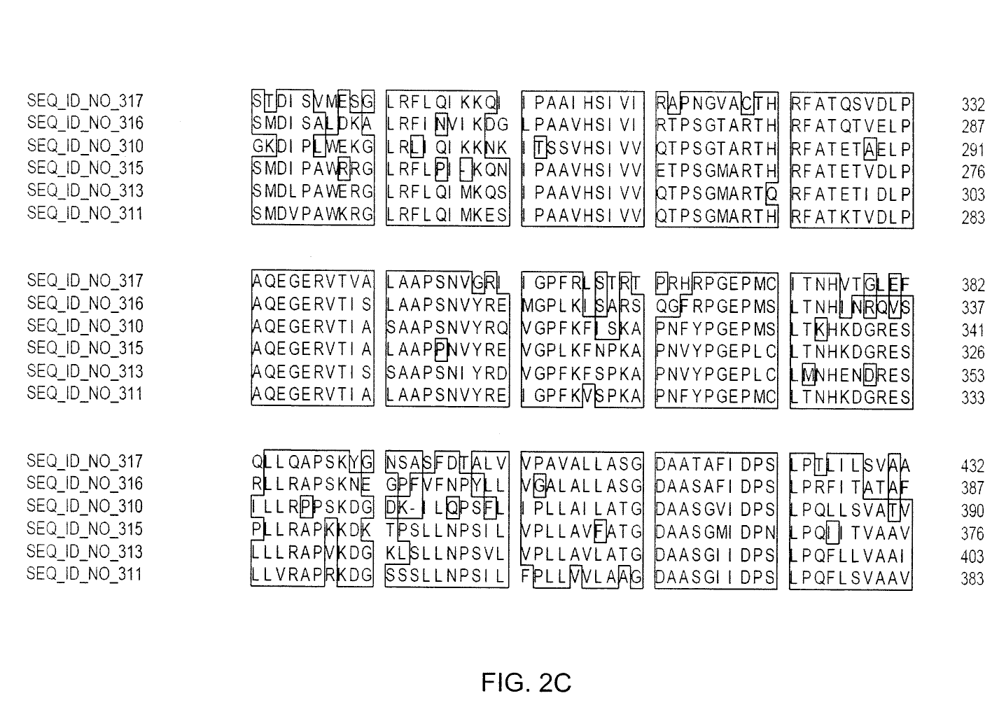

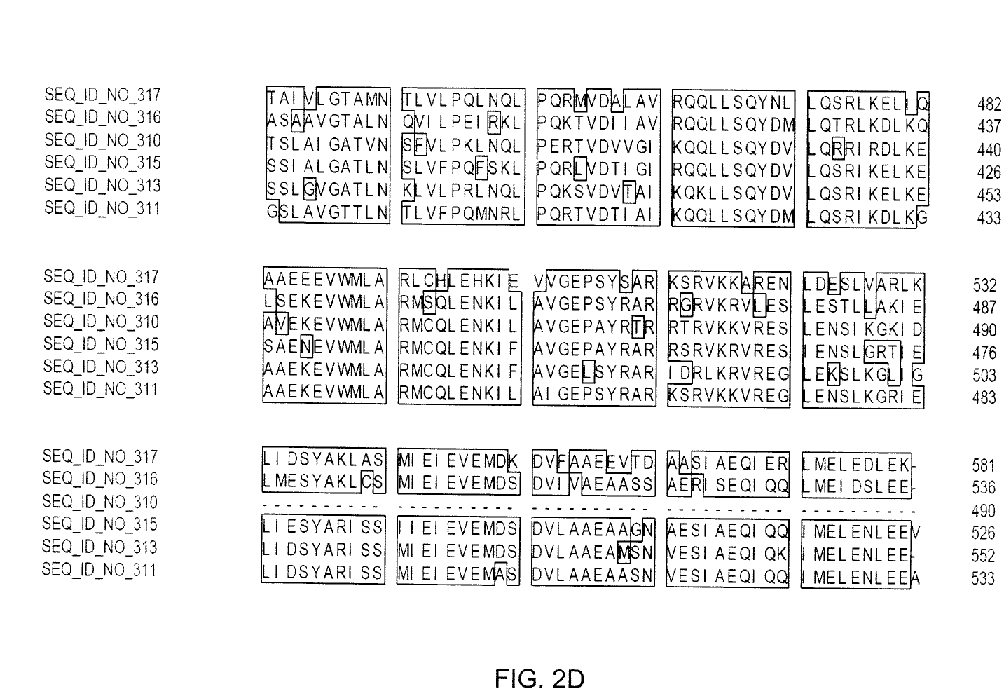

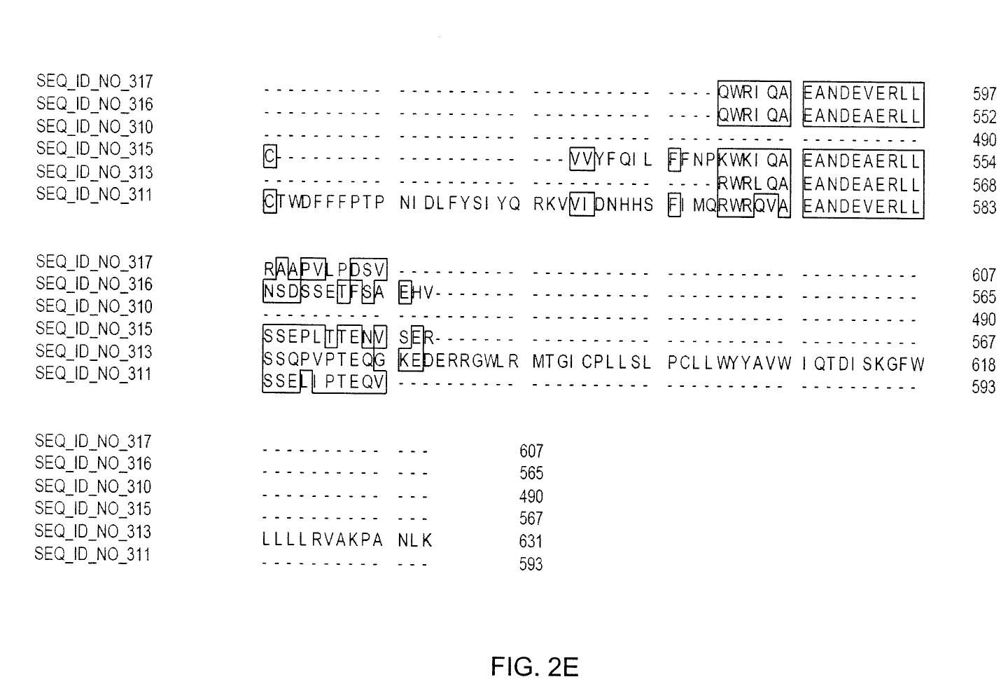

Examples of amino acid sequences of functional homologs of the polypeptide set forth in SEQ ID NO:310 are provided in FIG. 2 and in the Sequence Listing. Such functional homologs include, for example, GI:157360325 (SEQ ID NO:311), CeresAnnot:1500617 (SEQ ID NO:313), CeresAnnot:8457473 (SEQ ID NO:315), GI:115475946 (SEQ ID NO:316), GI:116789121 (SEQ ID NO:317), GI:12323467 (SEQ ID NO:318), GI:6633808 (SEQ ID NO:319), GI:147766222 (SEQ ID NO:320), CeresAnnot:1491360 (SEQ ID NO:322), CeresAnnot:1447617 (SEQ ID NO:324), CeresAnnot:1438527 (SEQ ID NO:326), CeresClone:1818368 (SEQ ID NO:328), CeresClone:1784441 (SEQ ID NO:330), GI:38424026 (SEQ ID NO:331), or CeresClone:860956 (SEQ ID NO:333). In some cases, a functional homolog of SEQ ID NO:310 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:310.

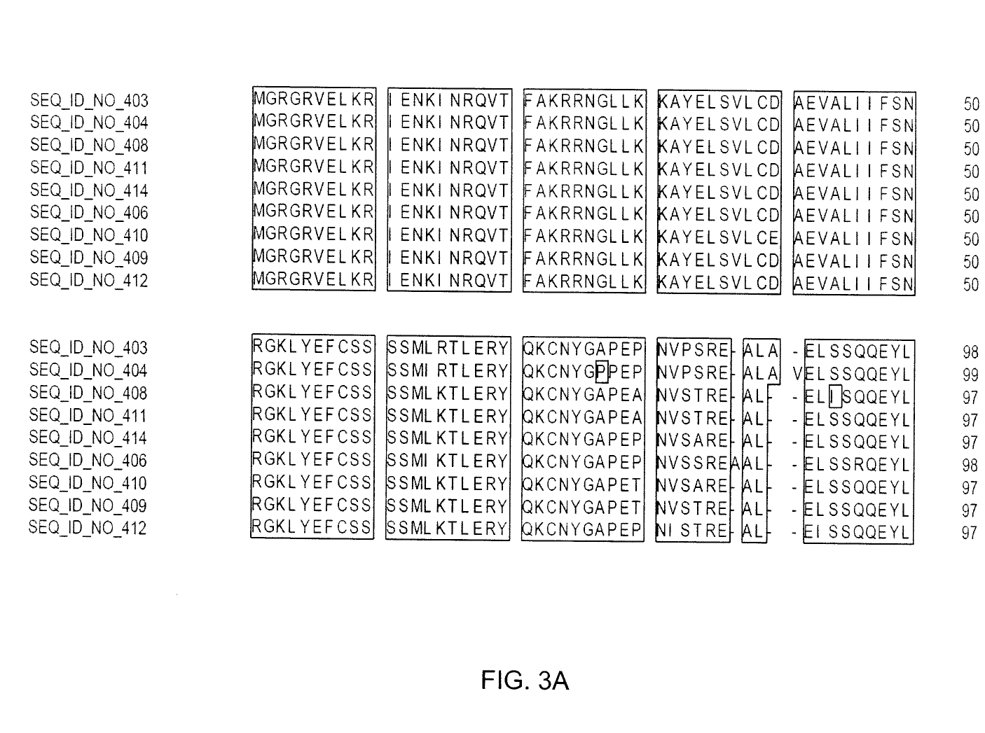

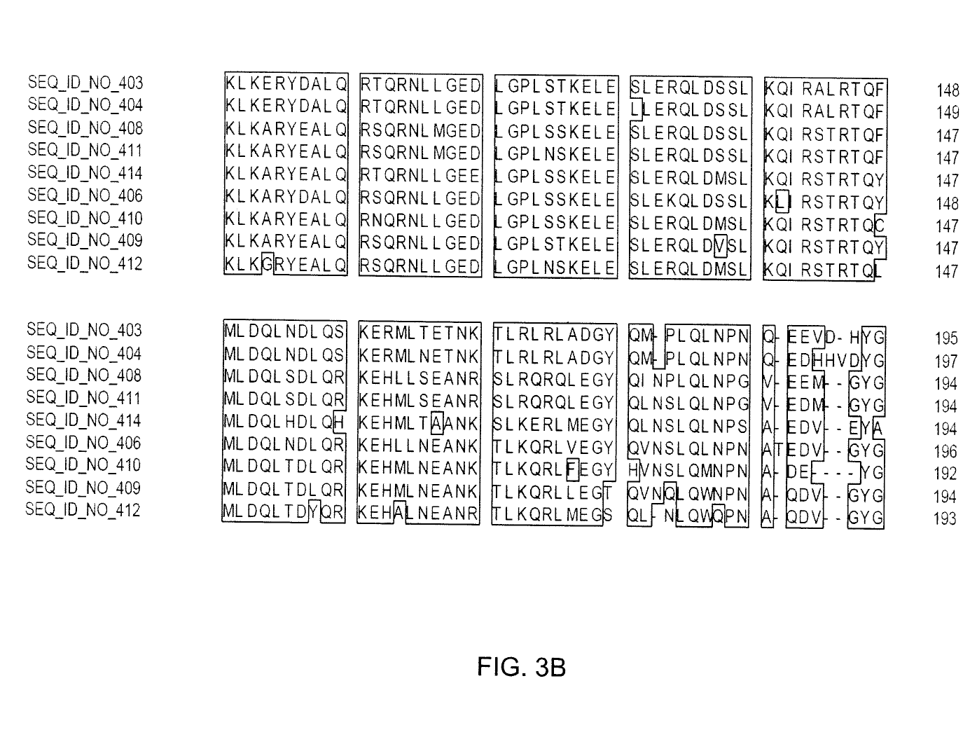

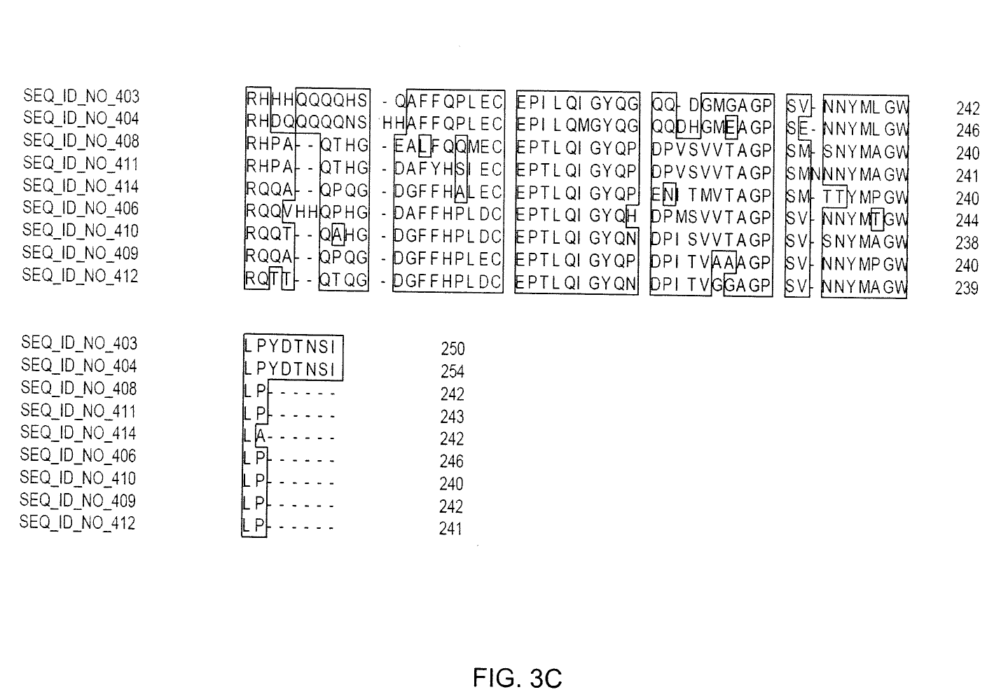

Examples of amino acid sequences of functional homologs of the polypeptide set forth in SEQ ID NO:403 are provided in FIG. 3 and in the Sequence Listing. Such functional homologs include, for example, GI:3912986 (SEQ ID NO:404), CeresClone:1935363 (SEQ ID NO:406), CeresClone:511680 (SEQ ID NO:408), GI:225425372 (SEQ ID NO:409), GI:126842923 (SEQ ID NO:410), GI:60100342 (SEQ ID NO:411), GI:31747210 (SEQ ID NO:412), CeresAnnot:1438157 (SEQ ID NO:414), GI:22329771 (SEQ ID NO:415), CeresClone:873647 (SEQ ID NO:417), CeresClone:1793863 (SEQ ID NO:419), GI:20385588 (SEQ ID NO:420), GI:58330675 (SEQ ID NO:421), GI:5070138 (SEQ ID NO:422), GI:1345965 (SEQ ID NO:423), GI:30314024 (SEQ ID NO:424), GI:75278480 (SEQ ID NO:425), GI:90903289 (SEQ ID NO:426), GI:1239959 (SEQ ID NO:427), GI:23194451 (SEQ ID NO:428), GI:121309556 (SEQ ID NO:429), GI:194247734 (SEQ ID NO:430), GI:116078097 (SEQ ID NO 431), GI:113207077 (SEQ ID NO:432), GI:87116791 (SEQ ID NO:433), GI:1239963 (SEQ ID NO:434), GI:114309696 (SEQ ID NO:435), GI:87045092 (SEQ ID NO:436), GI:27804361 (SEQ ID NO:437), GI:8745070 (SEQ ID NO:438), GI:87045090 (SEQ ID NO:439), GI:48727596 (SEQ ID NO:440), GI:148540546 (SEQ ID NO:441), GI:42491278 (SEQ ID NO:442), GI:60265532 (SEQ ID NO:443), GI:33309873 (SEQ ID NO:444), GI:34979580 (SEQ ID NO:445), GI:60265530 (SEQ ID NO:446), GI:85376980 (SEQ ID NO:447), GI:60265524 (SEQ ID NO:448), GI:117582132 (SEQ ID NO:449), GI:172034210 (SEQ ID NO:450), GI:99109356 (SEQ ID NO:451), GI:60265520 (SEQ ID NO:452), GI:172034212 (SEQ ID NO:453), GI:74053671 (SEQ ID NO:454), CeresClone:100031683 (SEQ ID NO:456), or GI:33309876 (SEQ ID NO:457). In some cases, a functional homolog of SEQ ID NO:403 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:403.

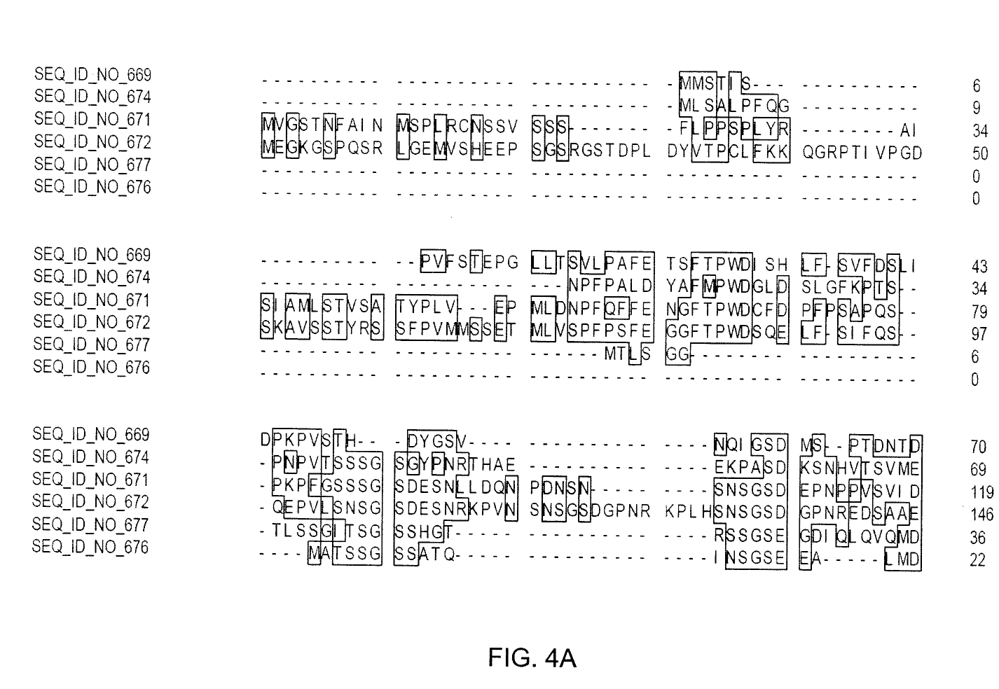

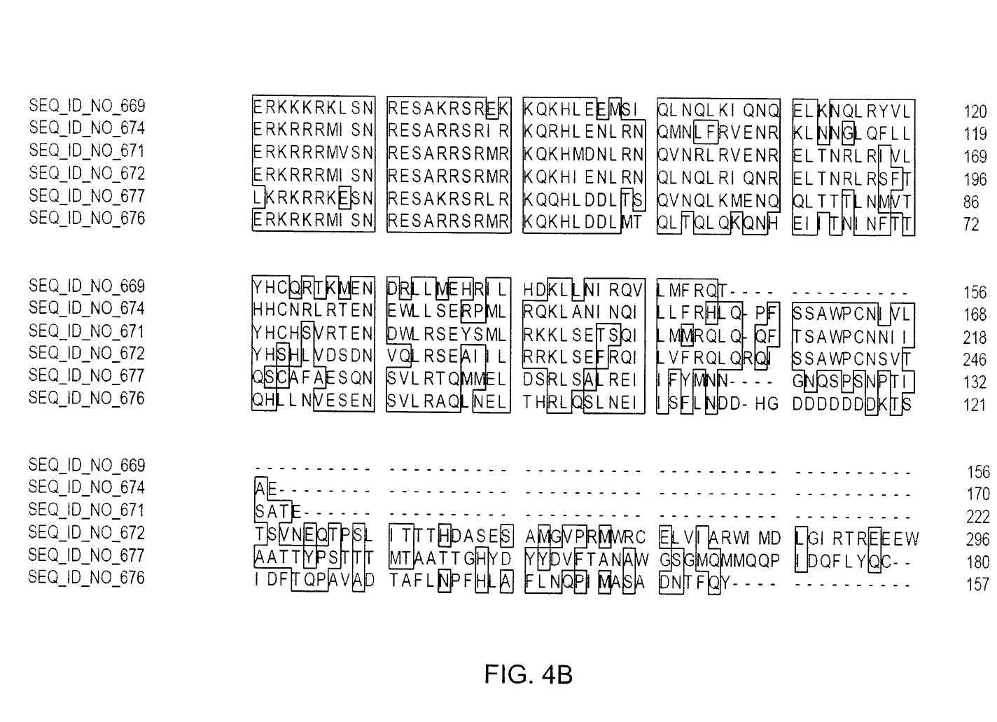

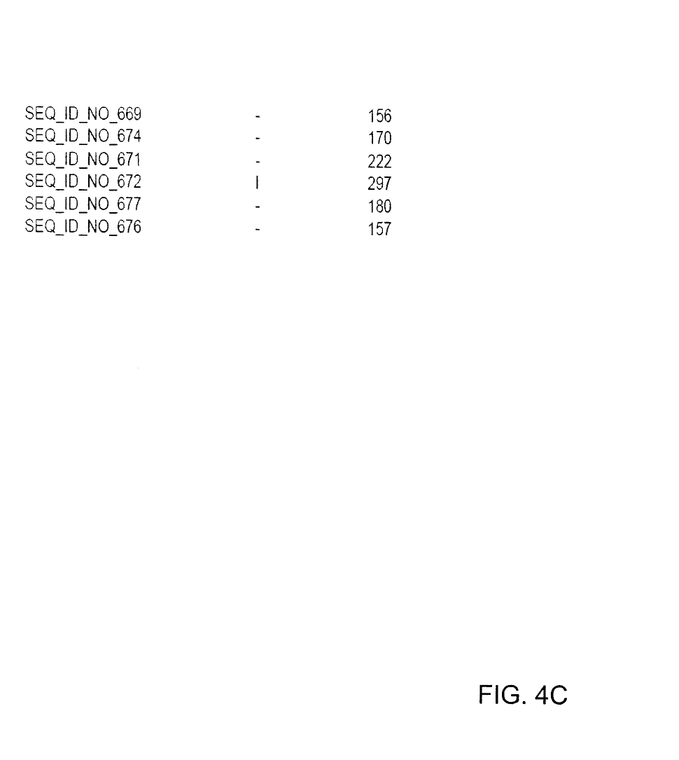

Examples of amino acid sequences of functional homologs of the polypeptide set forth in SEQ ID NO:669 are provided in FIG. 4 and in the Sequence Listing. Such functional homologs include, for example, CeresAnnot:1510674 (SEQ ID NO:671), GI:147825147 (SEQ ID NO:672), CeresClone:648620 (SEQ ID NO:674), CeresClone:1896520 (SEQ ID NO:676), GI:125603139 (SEQ ID NO:677), CeresAnnot:1470809 (SEQ ID NO:679), GI:157332804 (SEQ ID NO:680), CeresAnnot:1457468 (SEQ ID NO:682), CeresAnnot:552718 (SEQ ID NO:684), GI:125561247 (SEQ ID NO:685), CeresClone:1837887 (SEQ ID NO:687), CeresClone:1924353 (SEQ ID NO:689), GI:42563231 (SEQ ID NO:690), or GI:10120439 (SEQ ID NO:691). In some cases, a functional homolog of SEQ ID NO:669 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:669.

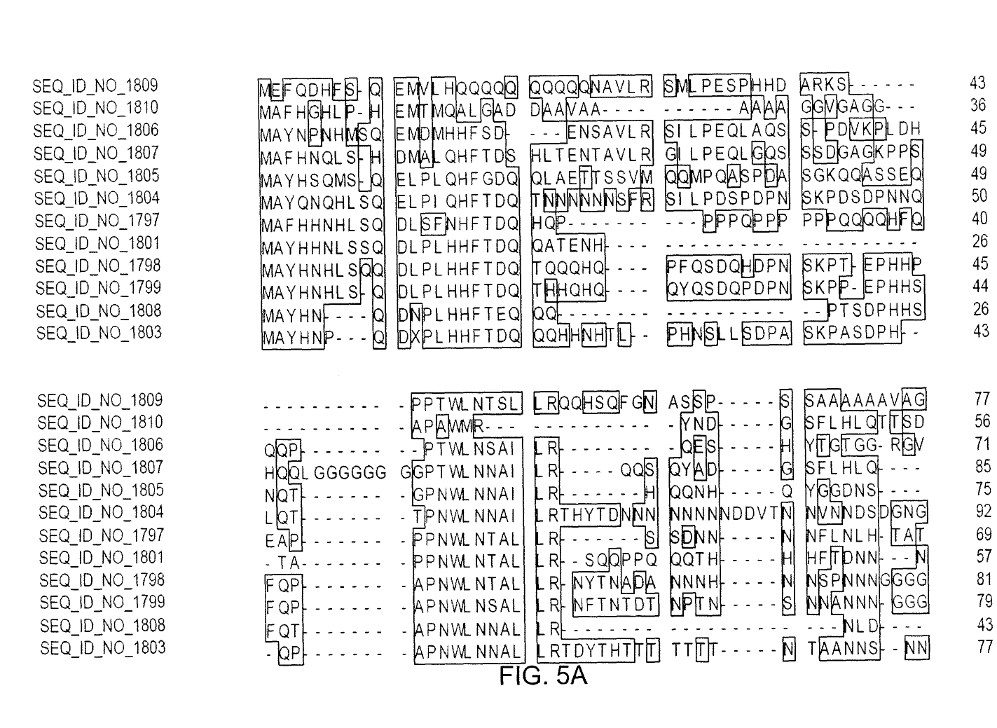

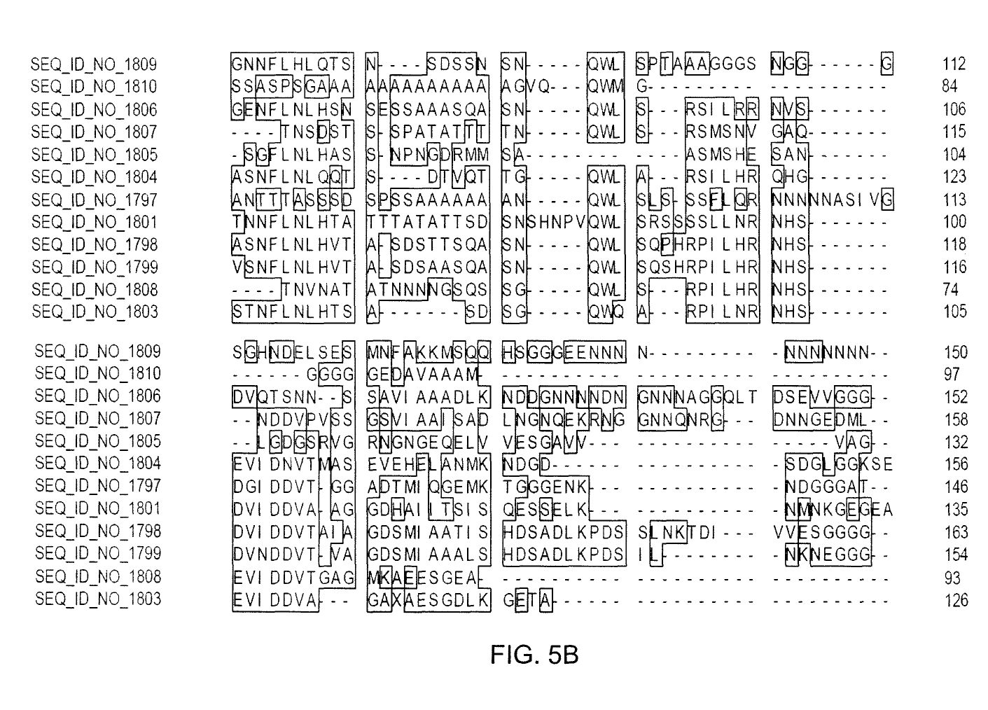

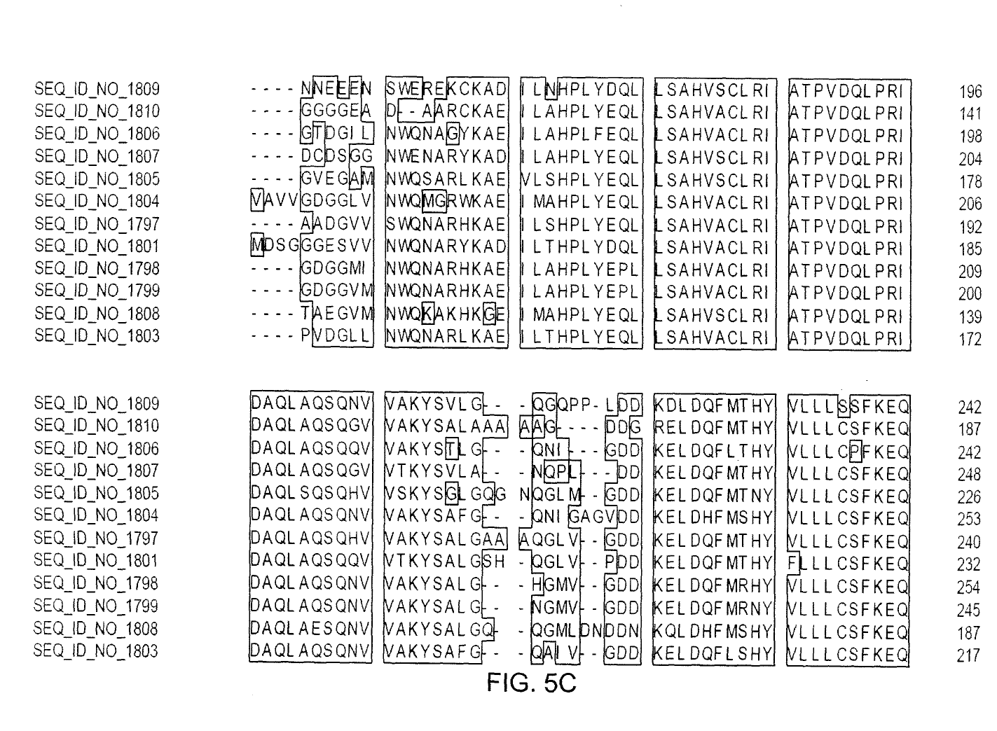

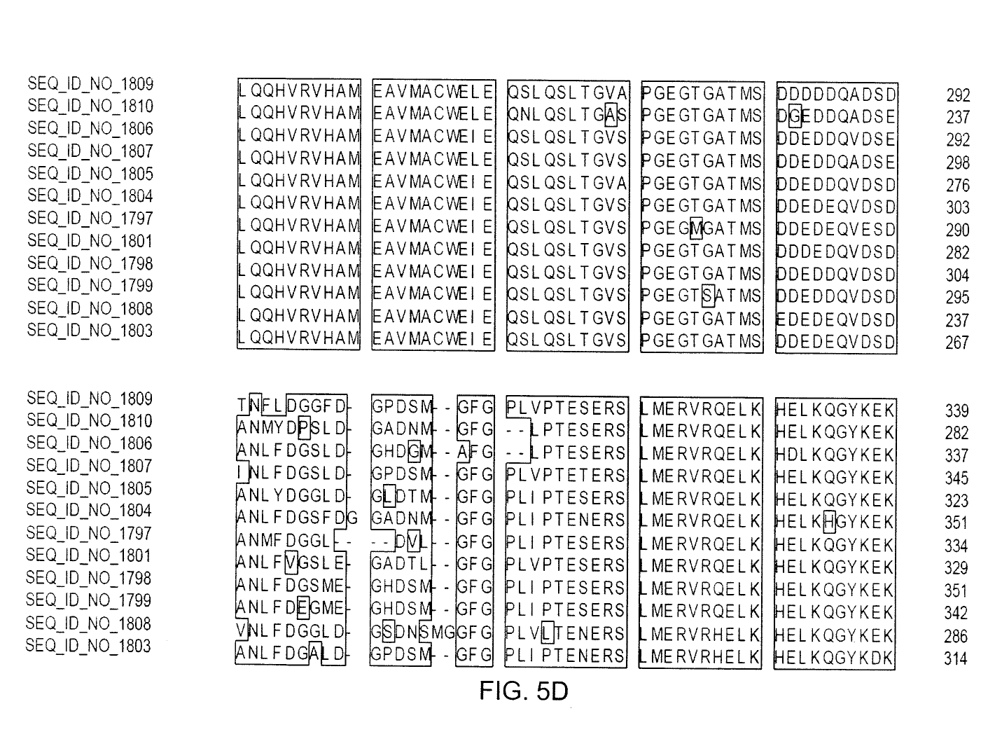

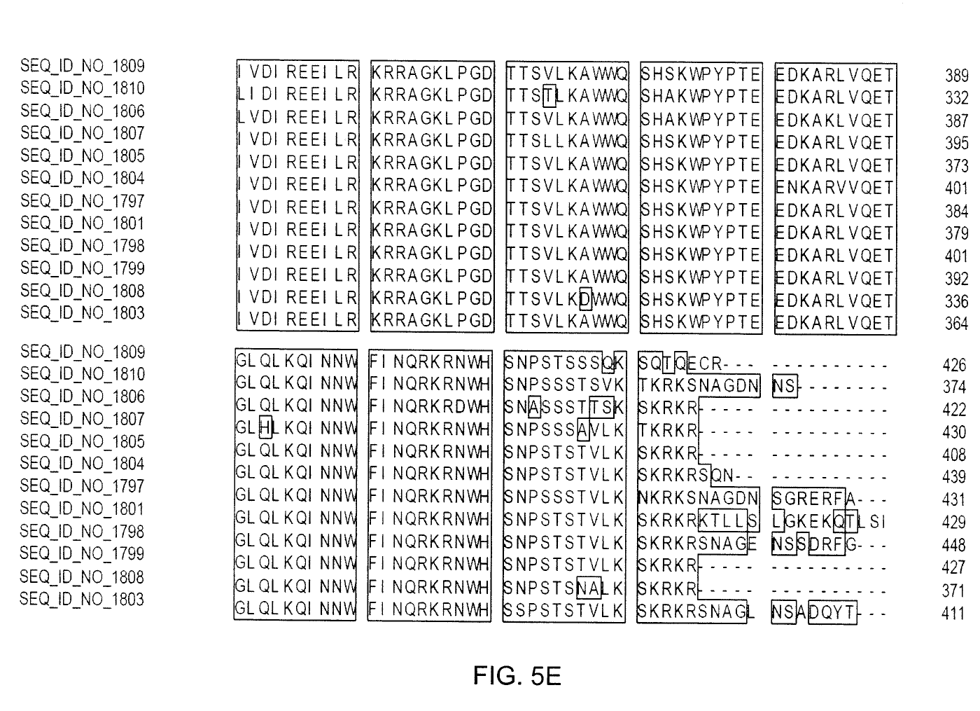

Examples of amino acid sequences of functional homologs of the polypeptide set forth in SEQ ID NO:1797 are provided in FIG. 5 and in the Sequence Listing. Such functional homologs include, for example GI:111038269 (SEQ ID NO:1798), GI:3023961 (SEQ ID NO:1799), CeresAnnot:1531214 (SEQ ID NO:1801), CeresClone:667754 (SEQ ID NO:1803), GI:132424655 (SEQ ID NO:1804), GI:167178664 (SEQ ID NO:1805), GI:3116212 (SEQ ID NO:1806), GI:225463014 (SEQ ID NO:1807), GI:132424659 (SEQ ID NO:1808), GI:6016226 (SEQ ID NO:1809), GI:221272018 (SEQ ID NO:1810), GI:15238743 (SEQ ID NO:1811), GI:79328660 (SEQ ID NO:1812), GI:1045044 (SEQ ID NO:1813), CeresAnnot:882143 (SEQ ID NO:1815), GI:21539535 (SEQ ID NO:1816), GI:9795158 (SEQ ID NO:1817), CeresAnnot:1508832 (SEQ ID NO:1819), CeresClone:1971046 (SEQ ID NO:1821), GI:163838594 (SEQ ID NO:1822), GI:168199347 (SEQ ID NO:1823), CeresClone:515966 (SEQ ID NO:1825), GI:1805617 (SEQ ID NO:1826), GI:167178668 (SEQ ID NO:1827), GI:168199344 (SEQ ID NO:1828), GI:157339759 (SEQ ID NO:1829), CeresAnnot:1531927 (SEQ ID NO:1831), CeresAnnot:1507861 (SEQ ID NO:1833), GI:15236649 (SEQ ID NO:1834), GI:11463943 (SEQ ID NO:1835), CeresClone:1848387 (SEQ ID NO:1837), CeresAnnot:8671843 (SEQ ID NO:1839), CeresAnnot:8743760 (SEQ ID NO:1841), GI:115469202 (SEQ ID NO:1842), CeresClone:1816333 (SEQ ID NO:1844), GI:162458143 (SEQ ID NO:1845), CeresClone:218346 (SEQ ID NO:1847), CeresClone:1834447 (SEQ ID NO:1849), GI:33333542 (SEQ ID NO:1850), CeresClone:106887 (SEQ ID NO:1852), GI:21618231 (SEQ ID NO:1853), GI:132424657 (SEQ ID NO:1854), GI:26451690 (SEQ ID NO:1855), GI:8493589 (SEQ ID NO:1856), GI:18407708 (SEQ ID NO:1857), GI:58011289 (SEQ ID NO:1858), GI:168040482 (SEQ ID NO:1859), CeresClone:1923949 (SEQ ID NO:1861), CeresAnnot:1478433 (SEQ ID NO:1863), CeresClone:1920037 (SEQ ID NO:1865), GI:225426739 (SEQ ID NO:1866), GI:14348597 (SEQ ID NO:1867), CeresClone:528991 (SEQ ID NO:1869), CeresAnnot:1491629 (SEQ ID NO:1871), GI:118481200 (SEQ ID NO:1872), CeresClone:298091 (SEQ ID NO:1874), CeresClone:558121 (SEQ ID NO:1876), GI:75306683 (SEQ ID NO:1877), CeresAnnot:8643472 (SEQ ID NO:1879), CeresClone:1824153 (SEQ ID NO:1881), GI:168026109 (SEQ ID NO:1882), GI:4098246 (SEQ ID NO:1883), or CeresClone:336323 (SEQ ID NO:1885). In some cases, a functional homolog of SEQ ID NO:1797 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:1797.

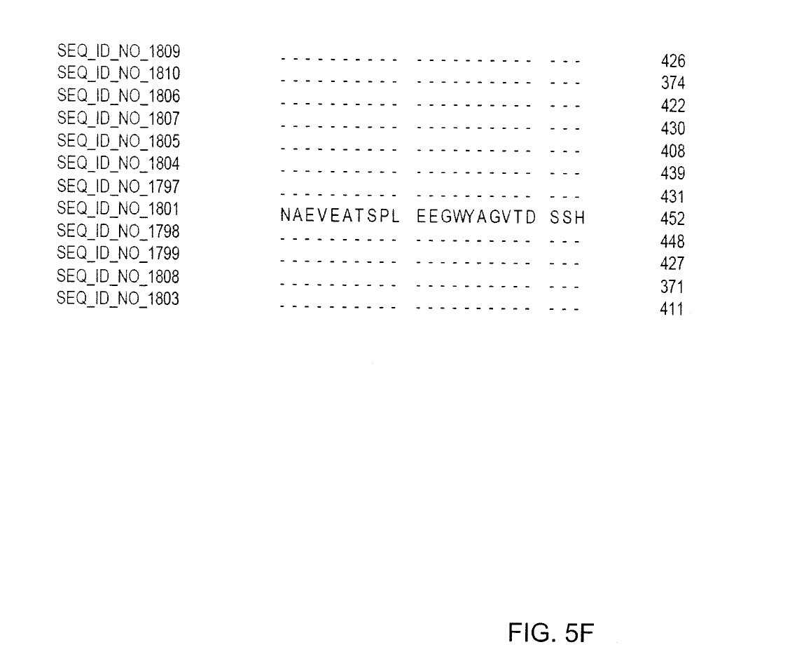

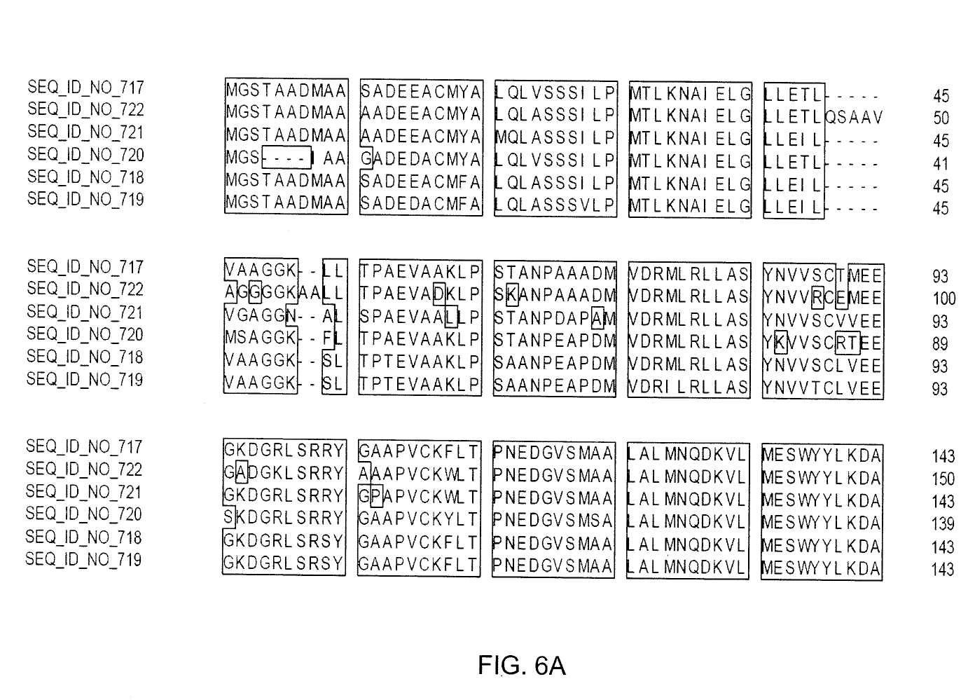

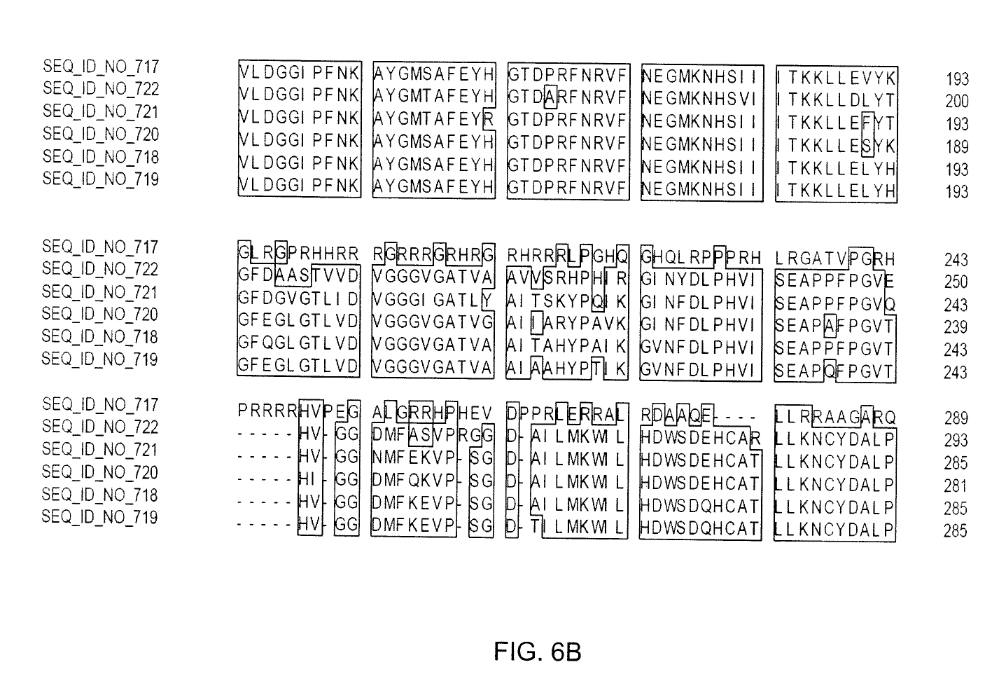

Examples of amino acid sequences of functional homologs of the polypeptide set forth in SEQ ID NO:717 are provided in FIG. 6 and in the Sequence Listing. Such functional homologs include, for example, GI:14578615 (SEQ ID NO:718), GI:4104220 (SEQ ID NO:719), GI:148337324 (SEQ ID NO:720), GI:151579841 (SEQ ID NO:721), GI:115474869 (SEQ ID NO:722), GI:30385246 (SEQ ID NO:723), CeresClone:1824196 (SEQ ID NO:725), GI:14578613 (SEQ ID NO:726), GI:14578611 (SEQ ID NO:727), GI:14578617 (SEQ ID NO:728), CeresClone:1453701 (SEQ ID NO:730), GI:77818928 (SEQ ID NO:731), GI:2388664 (SEQ ID NO:732), GI:145693796 (SEQ ID NO:733), GI:125560205 (SEQ ID NO:734), GI:125602246 (SEQ ID NO:735), CeresClone:1769444 (SEQ ID NO:737), CeresClone:1869486 (SEQ ID NO:739), CeresClone:1769168 (SEQ ID NO:741), CeresAnnot:8704591 (SEQ ID NO:743), GI:29839259 (SEQ ID NO:744), GI:18033964 (SEQ ID NO:745), GI:34398680 (SEQ ID NO:746), or GI:33641708 (SEQ ID NO:747). In some cases, a functional homolog of SEQ ID NO:717 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:717.

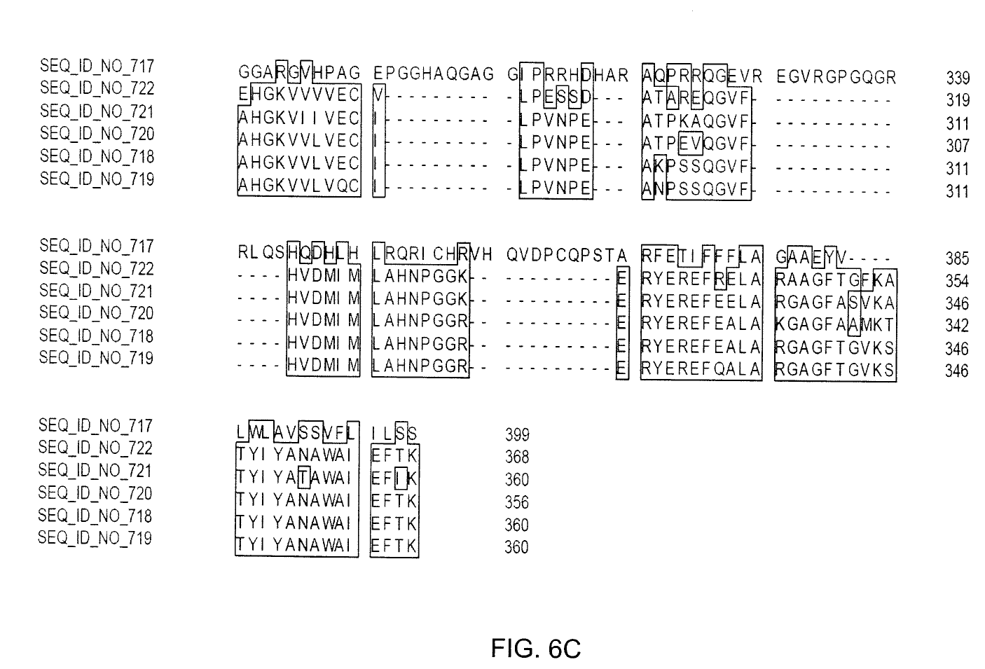

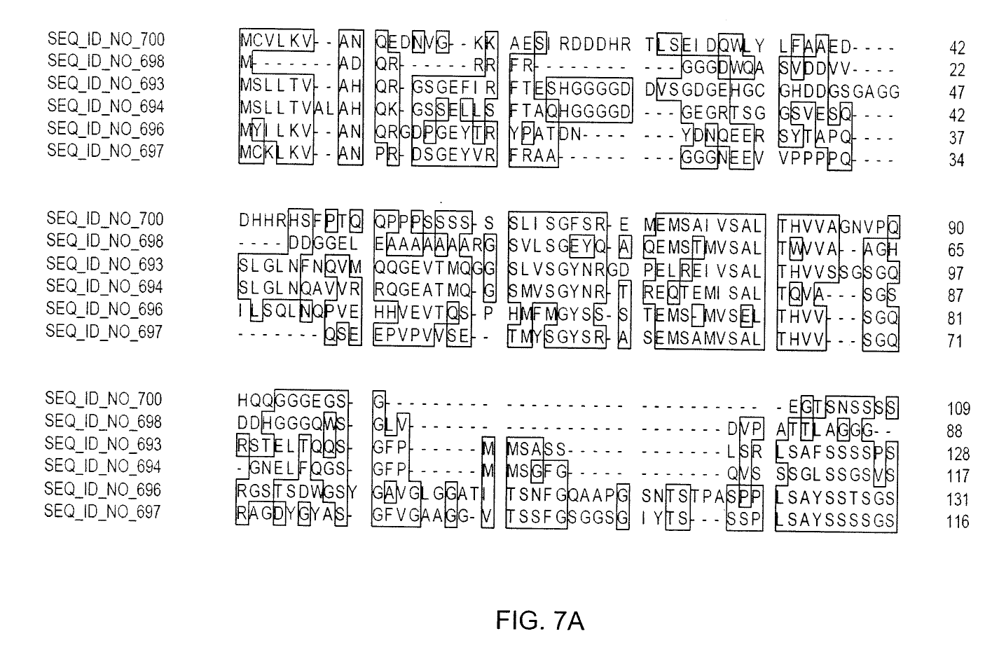

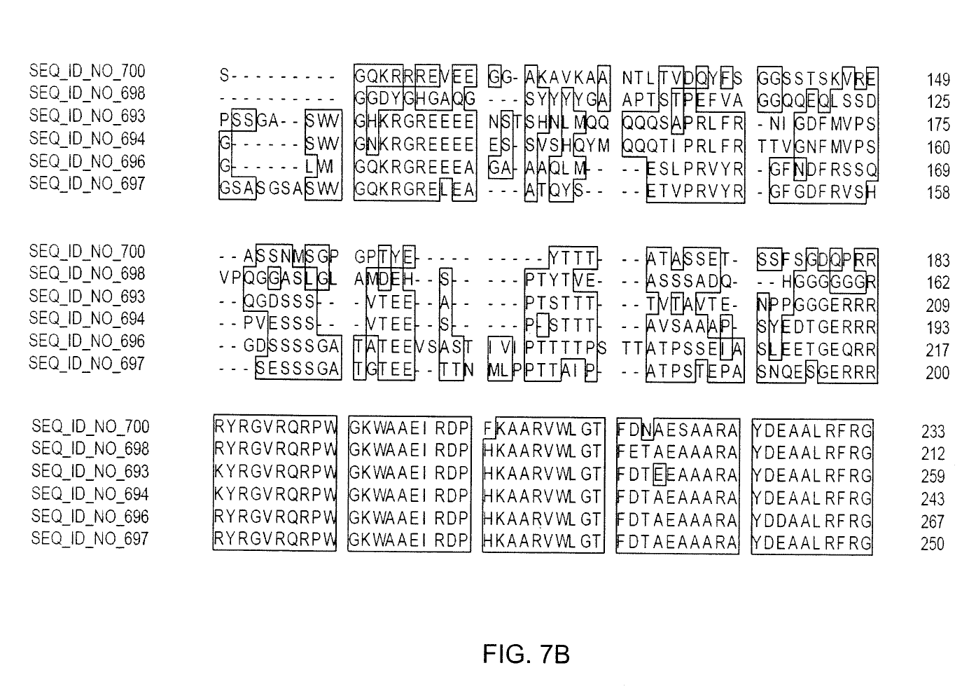

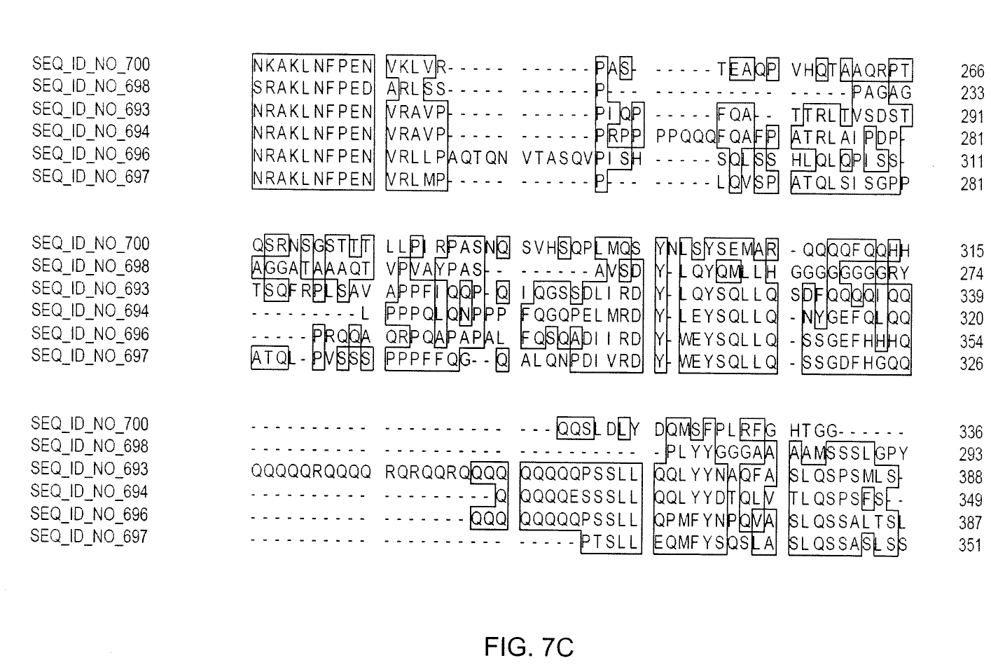

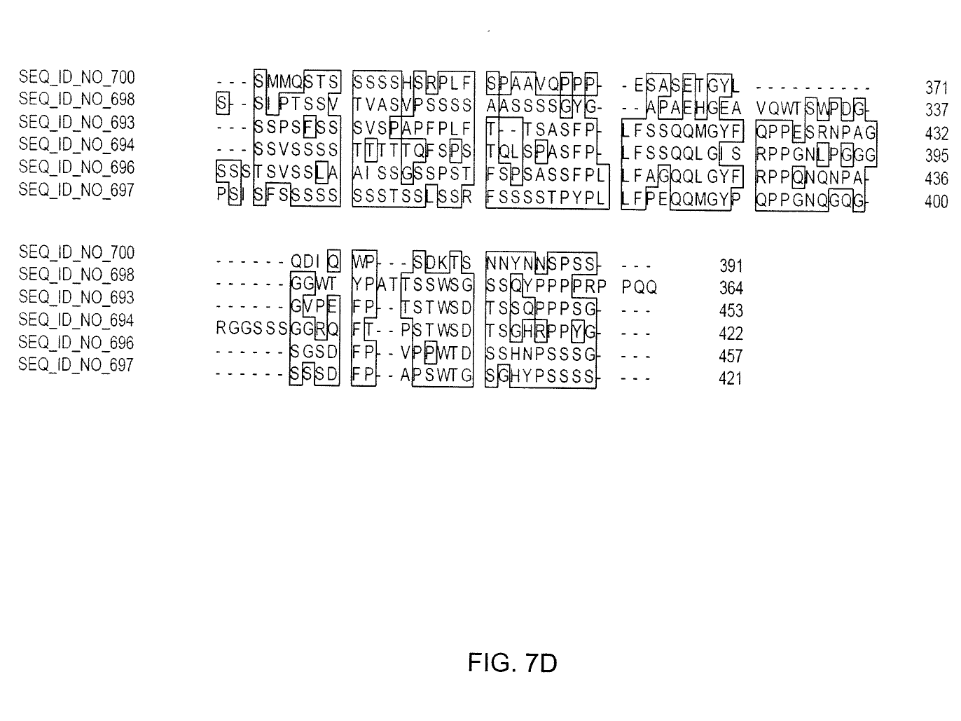

Examples of amino acid sequences of functional homologs of the polypeptide set forth in SEQ ID NO:693 are provided in FIG. 7 and in the Sequence Listing. Such functional homologs include, for example, GI:193237575 (SEQ ID NO:694), CeresAnnot:1443093 (SEQ ID NO:696), GI:147834261 (SEQ ID NO:697), GI:115464101 (SEQ ID NO:698), CeresAnnot:887551 (SEQ ID NO:700), CeresAnnot:1452324 (SEQ ID NO:702), CeresClone:1365932 (SEQ ID NO:704), GI:157352501 (SEQ ID NO:705), GI:225438481 (SEQ ID NO:706), GI:125533543 (SEQ ID NO:707), CeresAnnot:1451017 (SEQ ID NO:709), GI:115484369 (SEQ ID NO:710), CeresAnnot:1456607 (SEQ ID NO:712), CeresAnnot:8683331 (SEQ ID NO:714), or GI:115441277 (SEQ ID NO:715). In some cases, a functional homolog of SEQ ID NO:693 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:693.

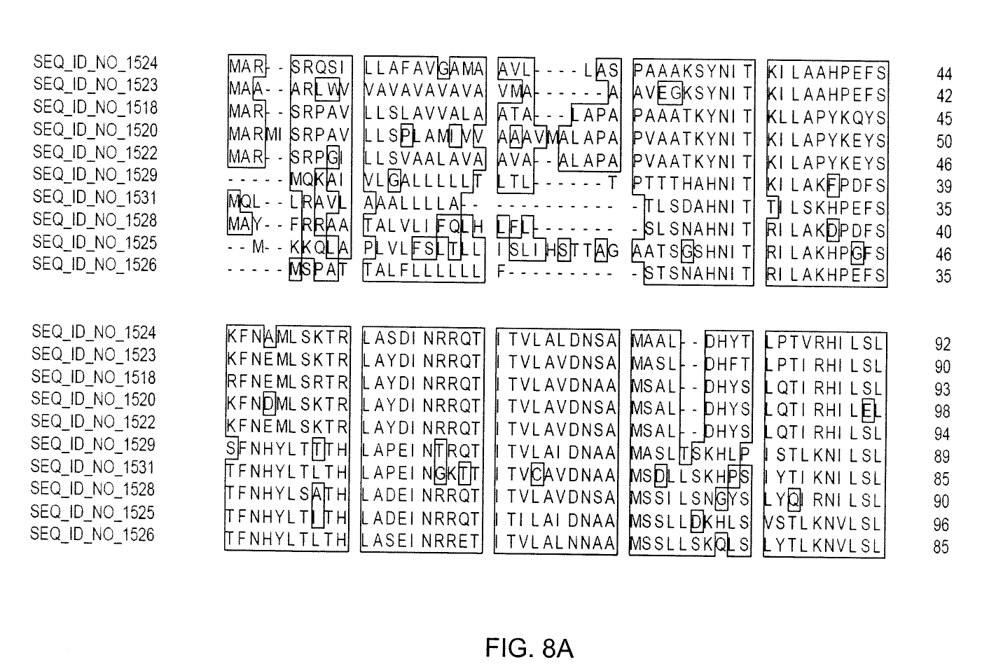

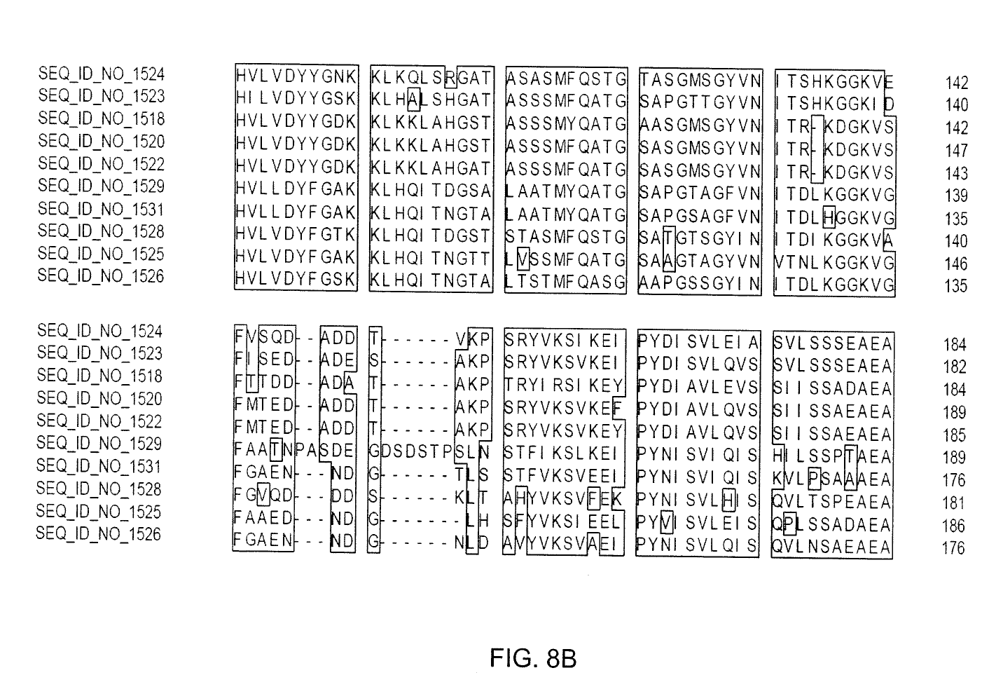

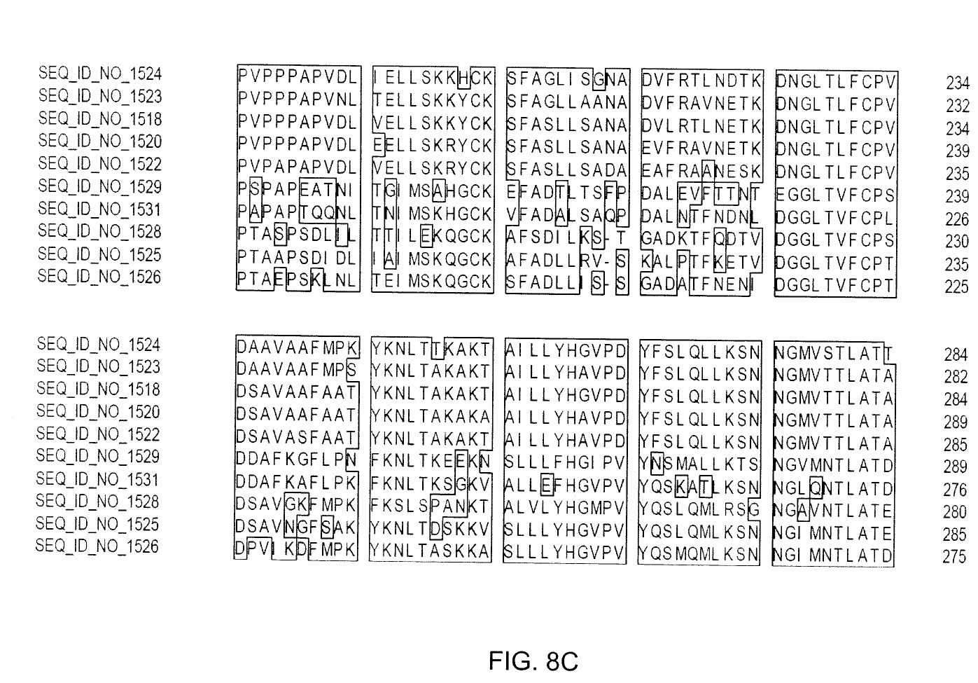

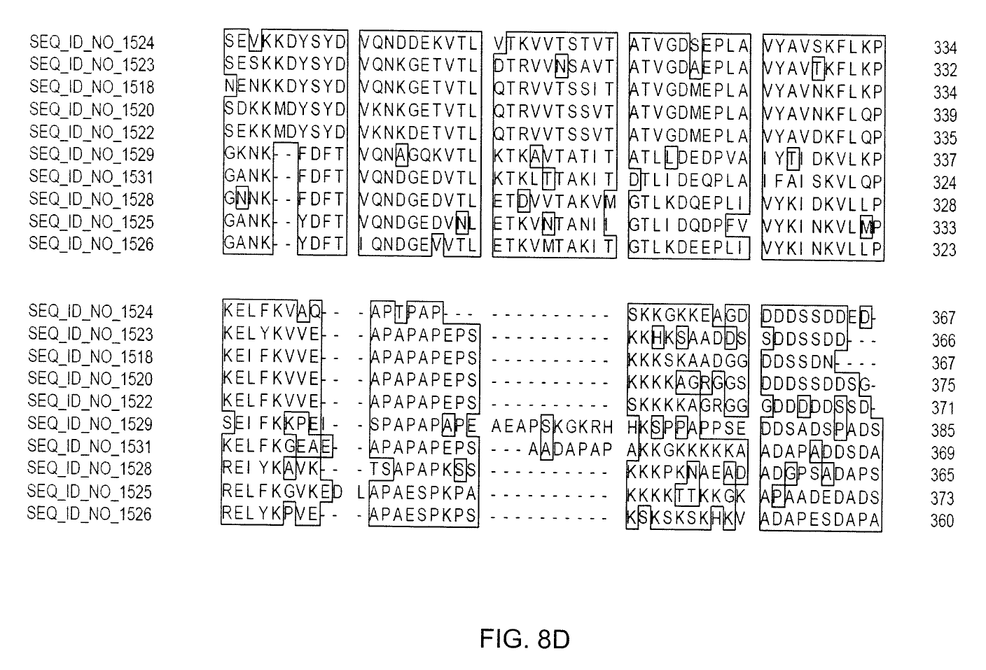

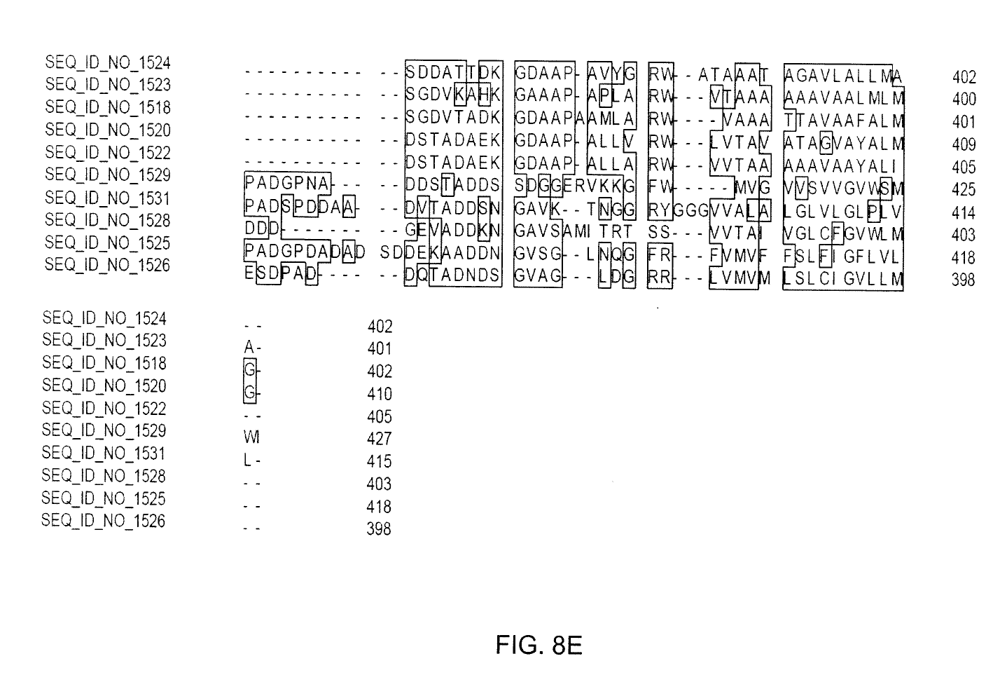

Examples of amino acid sequences of functional homologs of the polypeptide set forth in SEQ ID NO:1518 are provided in FIG. 8 and in the Sequence Listing. Such functional homologs include, for example, CeresClone:1671049 (SEQ ID NO:1520), CeresAnnot:8651870 (SEQ ID NO:1522), GI:115478142 (SEQ ID NO:1523), GI:115349940 (SEQ ID NO:1524), GI:84468390 (SEQ ID NO:1525), GI:157273672 (SEQ ID NO:1526), CeresClone:4620 (SEQ ID NO:1528), GI:84871616 (SEQ ID NO:1529), CeresClone:664500 (SEQ ID NO:1531), CeresClone:1995956 (SEQ ID NO:1533), GI:194706396 (SEQ ID NO:1534), GI:115475872 (SEQ ID NO:1535), CeresClone:700193 (SEQ ID NO:1537), GI:115349890 (SEQ ID NO:1538), CeresAnnot:8725185 (SEQ ID NO:1540), CeresClone:1409995 (SEQ ID NO:1542), CeresClone:1726596 (SEQ ID NO:1544), CeresClone:1790253 (SEQ ID NO:1546), GI:125602970 (SEQ ID NO:1547), CeresClone:1895671 (SEQ ID NO:1549), GI:13377778 (SEQ ID NO:1550), CeresClone:1929508 (SEQ ID NO:1552), CeresAnnot:1494487 (SEQ ID NO:1554), GI:147798736 (SEQ ID NO:1555), CeresAnnot:8461260 (SEQ ID NO:1557), GI:157273654 (SEQ ID NO:1558), CeresClone:1884727 (SEQ ID NO:1560), CeresAnnot:1466294 (SEQ ID NO:1562), GI:157273656 (SEQ ID NO:1563), GI:157351001 (SEQ ID NO:1564), GI:157353205 (SEQ ID NO:1565), or CeresClone:257067 (SEQ ID NO:1567). In some cases, a functional homolog of SEQ ID NO:1518 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:1518.

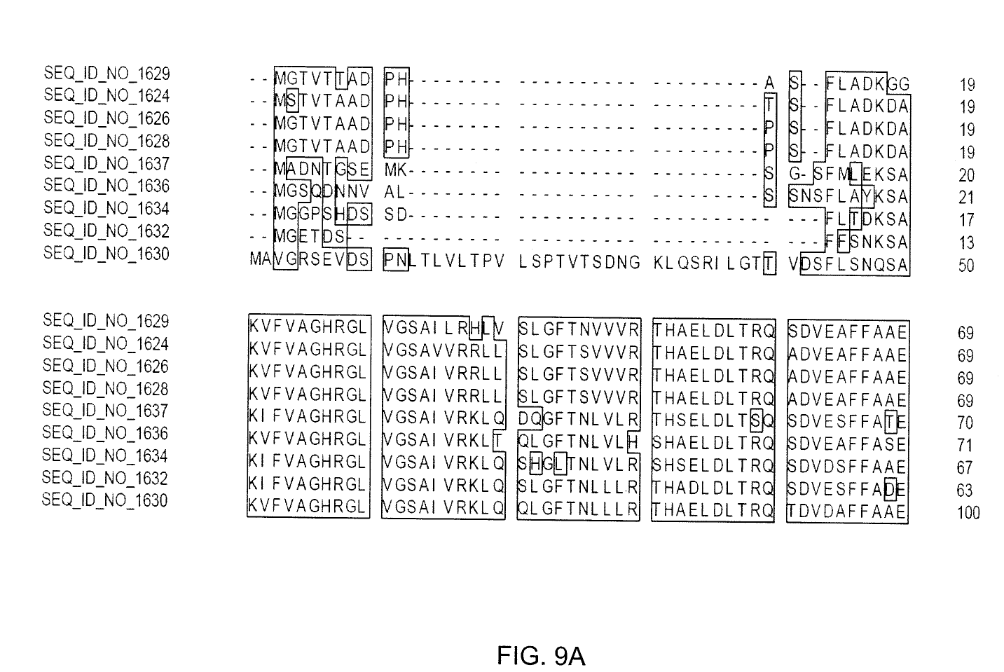

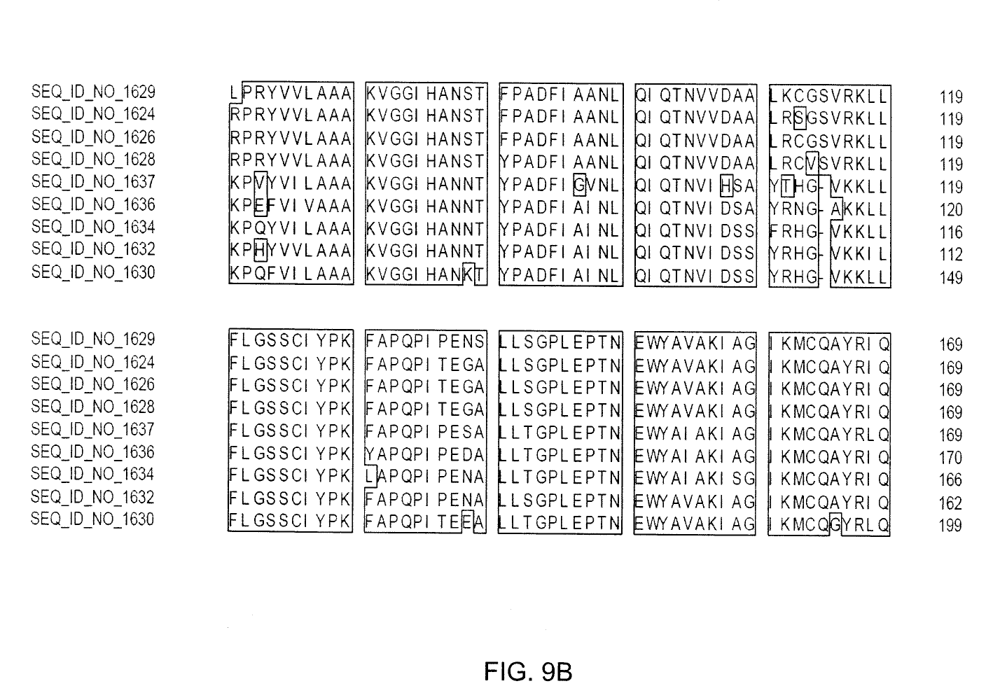

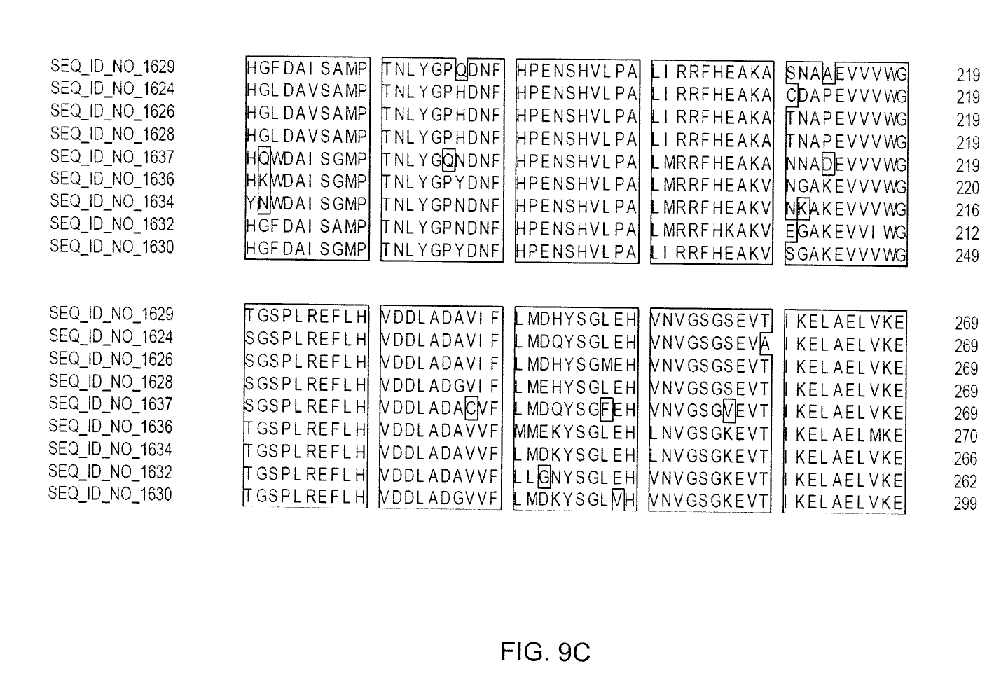

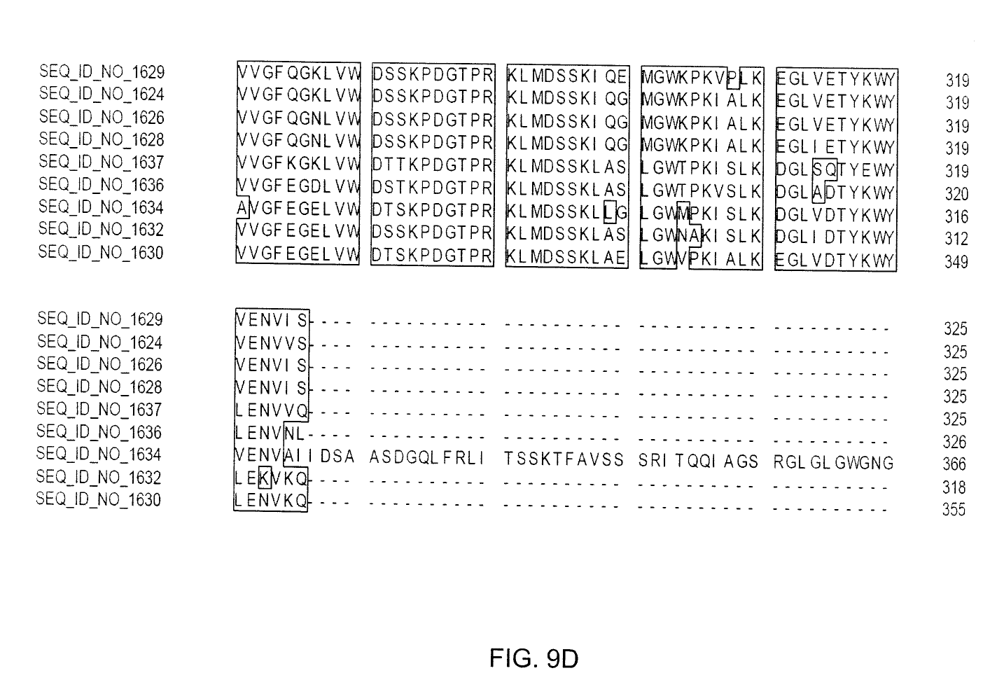

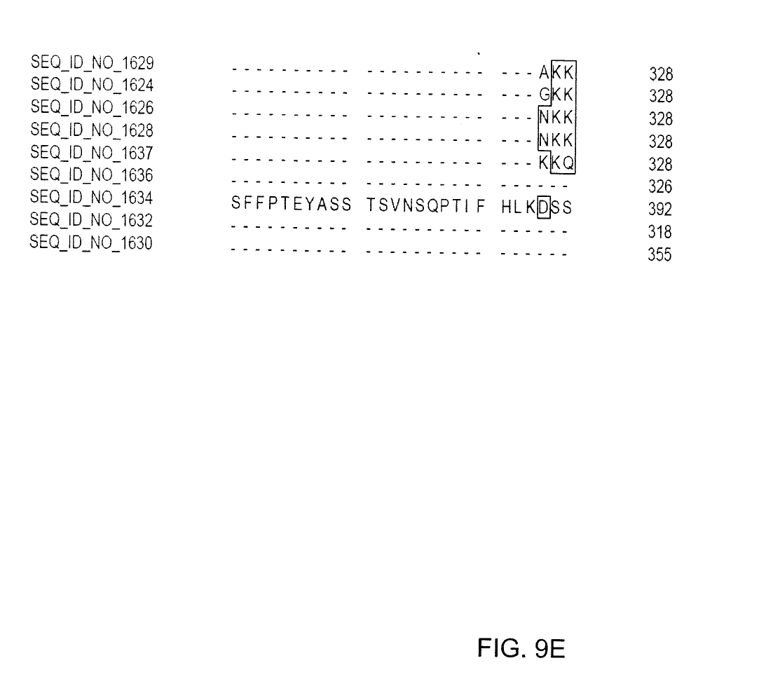

Examples of amino acid sequences of functional homologs of the polypeptide set forth in SEQ ID NO:1624 are provided in FIG. 9 and in the Sequence Listing. Such functional homologs include, for example, CeresClone:292606 (SEQ ID NO:1626), CeresAnnot:8743842 (SEQ ID NO:1628), GI:115469272 (SEQ ID NO:1629), GI:157343111 (SEQ ID NO:1630), CeresClone:1841546 (SEQ ID NO:1632), CeresAnnot:1478288 (SEQ ID NO:1634), CeresClone:463953 (SEQ ID NO:1636), GI:18394547 (SEQ ID NO:1637), CeresClone:2016499 (SEQ ID NO:1639), GI:125556307 (SEQ ID NO:1640), CeresClone:1996507 (SEQ ID NO:1642), CeresAnnot:1454854 (SEQ ID NO:1644), CeresClone:1393254 (SEQ ID NO:1646), GI:116788501 (SEQ ID NO:1647), GI:30698929 (SEQ ID NO:1648), GI:75098650 (SEQ ID NO:1649), GI:168058075 (SEQ ID NO:1650), CeresClone:19576 (SEQ ID NO:1652), CeresClone:1823703 (SEQ ID NO:1654), CeresAnnot:8743837 (SEQ ID NO:1656), GI:115469270 (SEQ ID NO:1657), or GI:125556305 (SEQ ID NO:1658). In some cases, a functional homolog of SEQ ID NO:1624 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:1624.

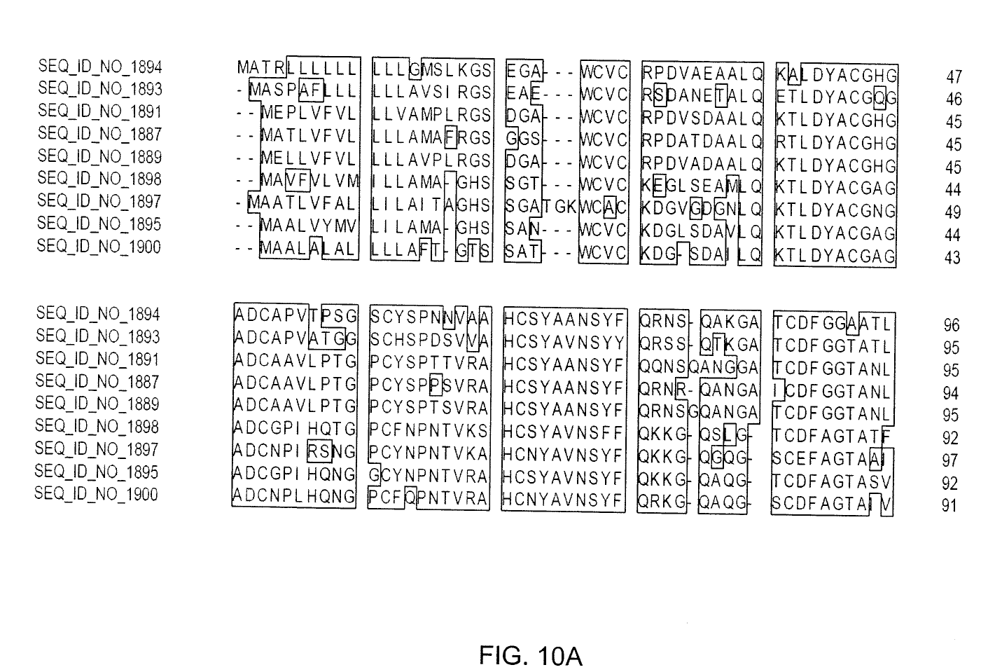

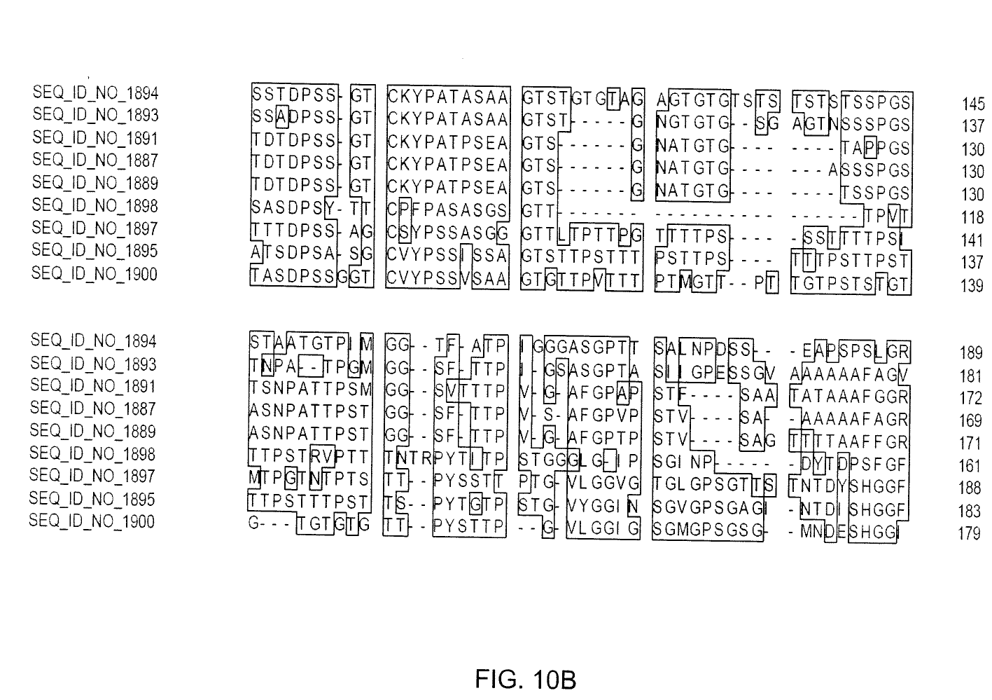

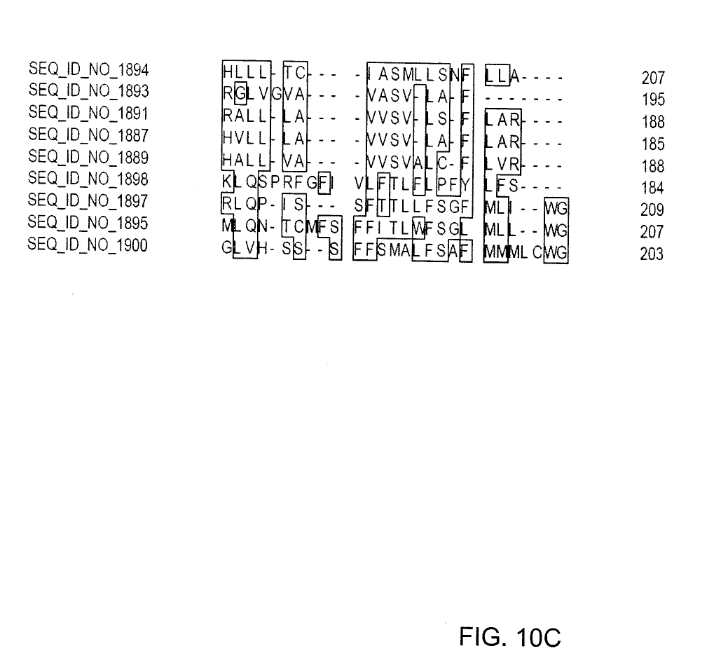

Examples of amino acid sequences of functional homologs of the polypeptide set forth in SEQ ID NO:1887 are provided in FIG. 10 and in the Sequence Listing. Such functional homologs include, for example, CeresClone:1724783 (SEQ ID NO:1889), CeresClone:1724784 (SEQ ID NO:1891), CeresClone:1724785 (SEQ ID NO:1893), CeresClone:1724786 (SEQ ID NO:1894), CeresClone:1724787 (SEQ ID NO:1895), CeresClone:1724788 (SEQ ID NO:1897), CeresClone:1724789 (SEQ ID NO:1898), CeresClone:1724790 (SEQ ID NO:1900), CeresClone:1724791 (SEQ ID NO:1902), CeresClone:1724792 (SEQ ID NO:1903), CeresClone:1724793 (SEQ ID NO:1904), CeresClone:1724794 (SEQ ID NO:1906), CeresClone:1724795 (SEQ ID NO:1908), CeresClone:1724796 (SEQ ID NO:1910), CeresClone:1724797 (SEQ ID NO:1912), CeresClone:1724798 (SEQ ID NO:1913), CeresClone:1724799 (SEQ ID NO:1915), CeresClone:1724800 (SEQ ID NO:1917), CeresClone:1724801 (SEQ ID NO:1918), CeresClone:1724802 (SEQ ID NO:1919), CeresClone:1724803 (SEQ ID NO:1920), CeresClone:1724804 (SEQ ID NO:1922), CeresClone:1724805 (SEQ ID NO:1924), CeresClone:1724806 (SEQ ID NO:1926), CeresClone:1724807 (SEQ ID NO:1928), CeresClone:1724808 (SEQ ID NO:1929), CeresClone:1724809 (SEQ ID NO:1931), CeresClone:1724810 (SEQ ID NO:1933), CeresClone:1724811 (SEQ ID NO:1935), CeresClone:1724812 (SEQ ID NO:1936), CeresClone:1724813 (SEQ ID NO:1938), CeresClone:1724814 (SEQ ID NO:1940), CeresClone:1724815 (SEQ ID NO:1942), or CeresClone:1724816 (SEQ ID NO:1944). In some cases, a functional homolog of SEQ ID NO:1887 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:1887.

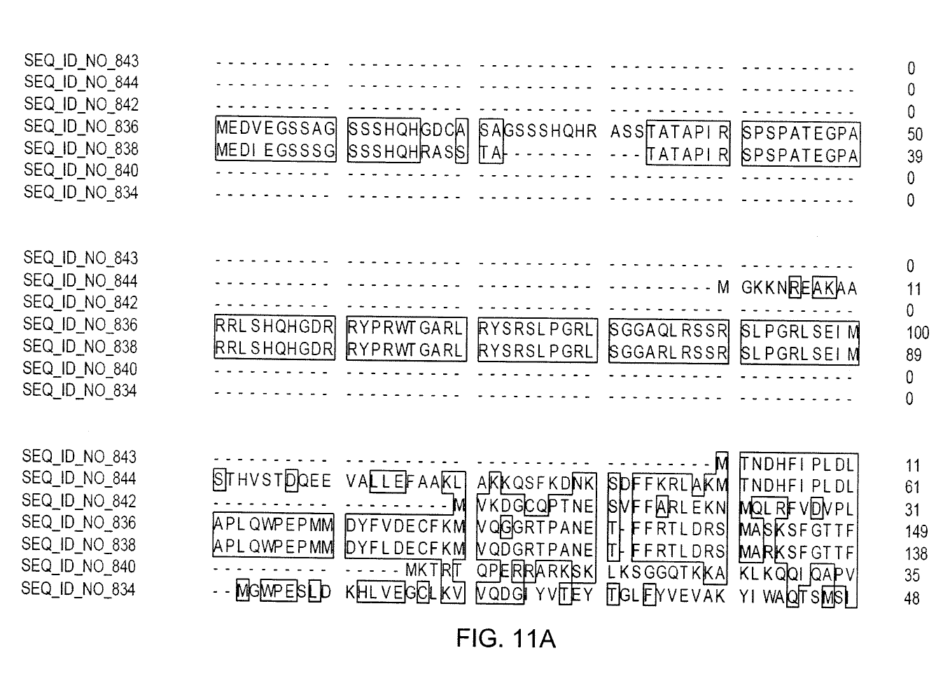

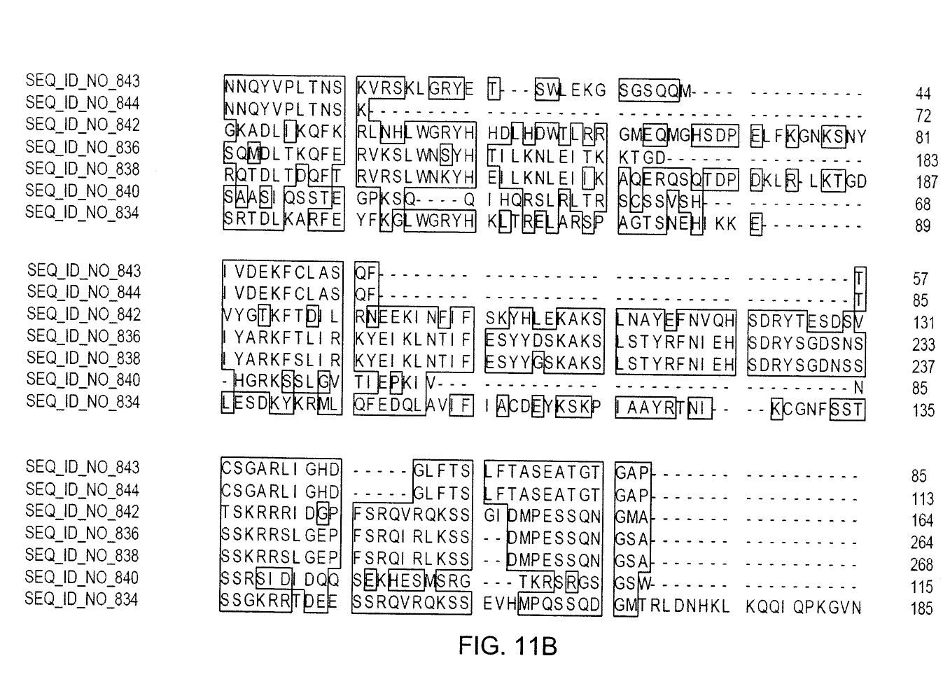

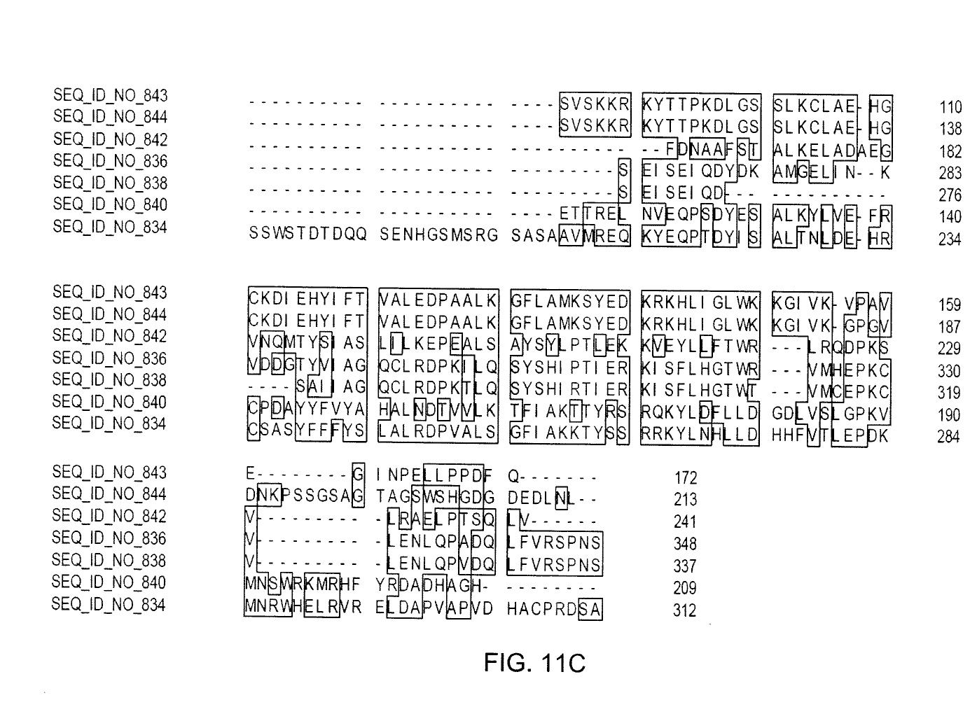

Examples of amino acid sequences of functional homologs of the polypeptide set forth in SEQ ID NO:834 are provided in FIG. 11 and in the Sequence Listing. Such functional homologs include, for example, CeresAnnot:8701141 (SEQ ID NO:836), CeresClone:917740 (SEQ ID NO:838), CeresClone:101143919 (SEQ ID NO:840), CeresClone:1858677 (SEQ ID NO:842), GI:125571017 (SEQ ID NO:843), or GI:53791578 (SEQ ID NO:844). In some cases, a functional homolog of SEQ ID NO:834 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:834.

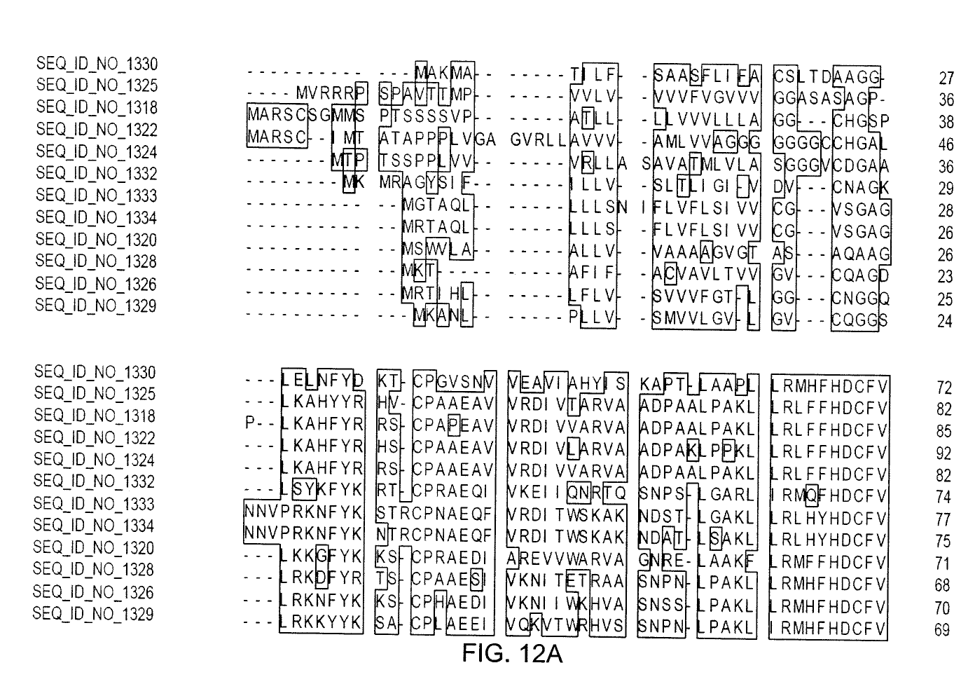

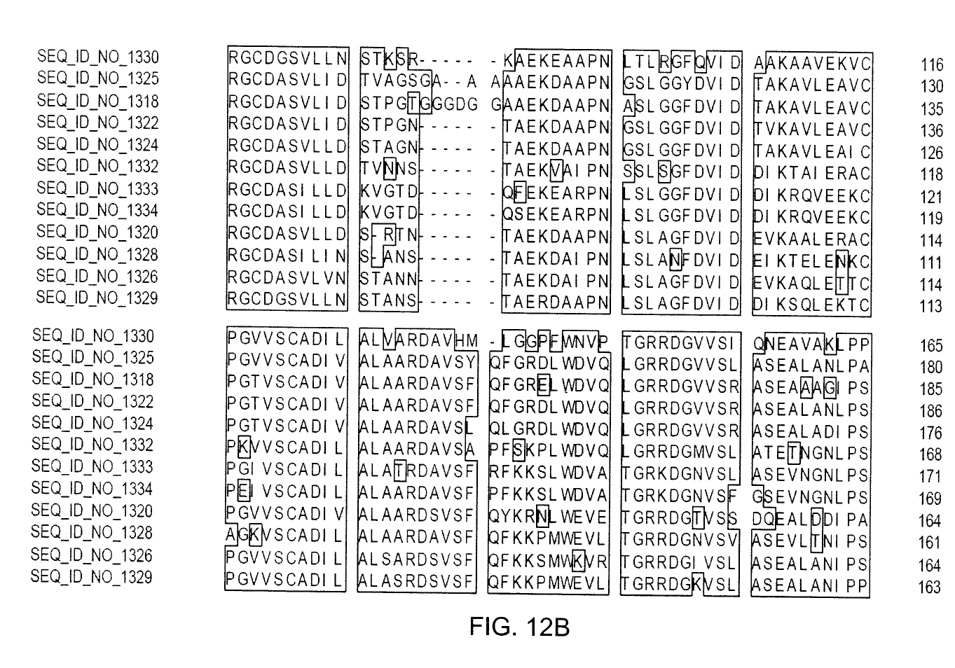

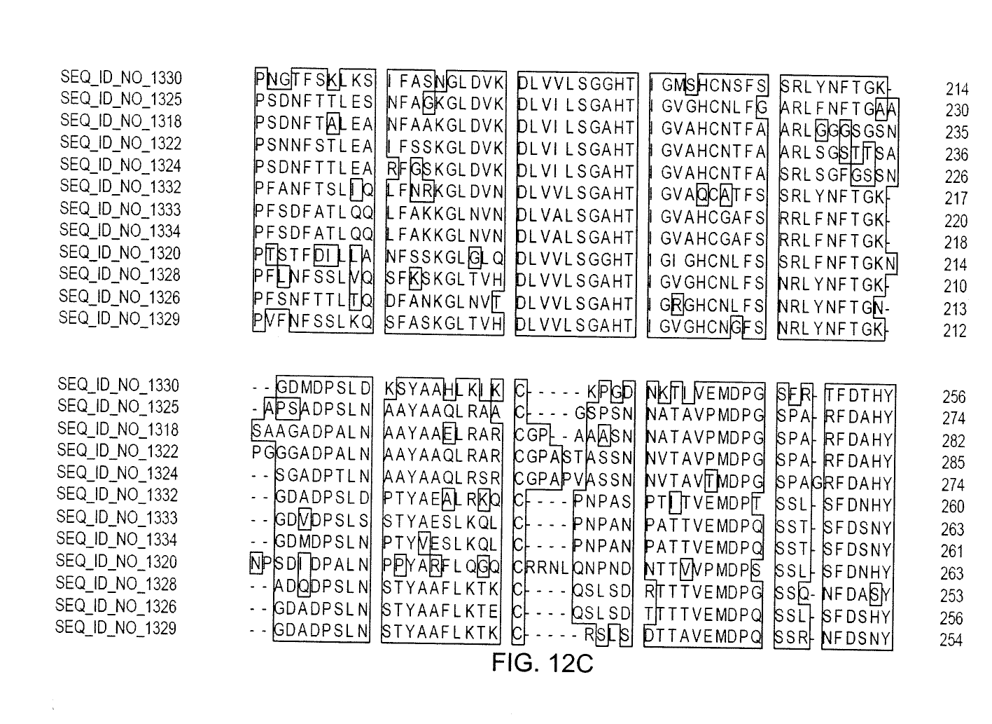

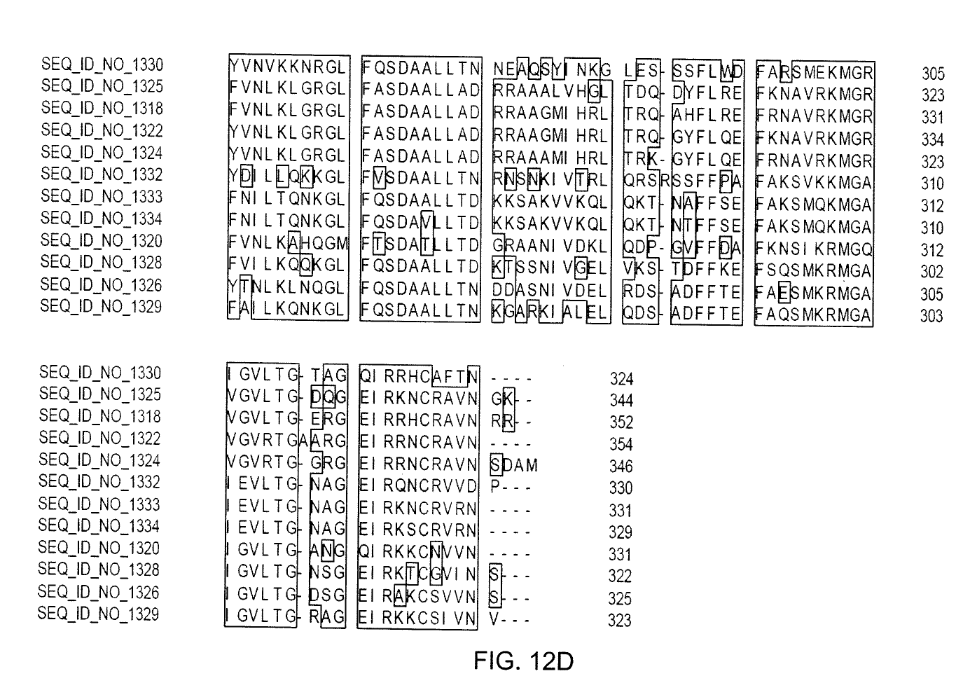

Examples of amino acid sequences of functional homologs of the polypeptide set forth in SEQ ID NO:1318 are provided in FIG. 12 and in the Sequence Listing. Such functional homologs include, for example, CeresClone:1816881 (SEQ ID NO:1320), CeresClone:100825990 (SEQ ID NO:1322), CeresAnnot:8722479 (SEQ ID NO:1324), GI:55701143 (SEQ ID NO:1325), GI:146335701 (SEQ ID NO:1326), CeresAnnot:1467253 (SEQ ID NO:1328), GI:225436825 (SEQ ID NO:1329), GI:116793602 (SEQ ID NO:1330), CeresClone:1922578 (SEQ ID NO:1332), GI:575605 (SEQ ID NO:1333), GI:575603 (SEQ ID NO:1334), CeresClone:1725430 (SEQ ID NO:1336), CeresClone:1402519 (SEQ ID NO:1338), GI:146289957 (SEQ ID NO:1339), GI:115436654 (SEQ ID NO:1340), GI:122726082 (SEQ ID NO:1341), GI:225432418 (SEQ ID NO:1342), GI:157336355 (SEQ ID NO:1343), GI:115469560 (SEQ ID NO:1344), GI:55701045 (SEQ ID NO:1345), CeresClone:1528781 (SEQ ID NO:1347), CeresAnnot:8632045 (SEQ ID NO:1349), GI:194701190 (SEQ ID NO:1350), CeresAnnot:8659657 (SEQ ID NO:1352), GI:125535203 (SEQ ID NO:1353), CeresClone:293854 (SEQ ID NO:1355), CeresClone:1559089 (SEQ ID NO:1357), GI:115486535 (SEQ ID NO:1358), GI:115467740 (SEQ ID NO:1359), GI:125555057 (SEQ ID NO:1360), GI:15225011 (SEQ ID NO:1361), CeresAnnot:8643832 (SEQ ID NO:1363), GI:118484065 (SEQ ID NO:1364), CeresAnnot:1488035 (SEQ ID NO:1366), GI:51970718 (SEQ ID NO:1367), CeresAnnot:8464351 (SEQ ID NO:1369), GI:115435890 (SEQ ID NO:1370), GI:115472505 (SEQ ID NO:1371), GI:125558613 (SEQ ID NO:1372), GI:225447326 (SEQ ID NO:1373), CeresClone:909358 (SEQ ID NO:1375), GI:115444399 (SEQ ID NO:1376), GI:125538188 (SEQ ID NO:1377), CeresAnnot:8457856 (SEQ ID NO:1379), GI:15220463 (SEQ ID NO:1380), GI:21536908 (SEQ ID NO:1381), GI:50261255 (SEQ ID NO:1382), CeresClone:757325 (SEQ ID NO:1384), CeresAnnot:8643830 (SEQ ID NO:1386), CeresAnnot:8744201 (SEQ ID NO:1388), GI:55700915 (SEQ ID NO:1389), CeresClone:1923915 (SEQ ID NO:1391), CeresClone:114862 (SEQ ID NO:1393), CeresAnnot:1509237 (SEQ ID NO:1395), CeresClone:100906383 (SEQ ID NO:1397), CeresClone:1796942 (SEQ ID NO:1399), CeresClone:1777201 (SEQ ID NO:1401), GI:54290444 (SEQ ID NO:1402), GI:147772815 (SEQ ID NO:1403), CeresClone:280031 (SEQ ID NO:1405), CeresAnnot:1456042 (SEQ ID NO:1407), CeresClone:39952 (SEQ ID NO:1409), GI:225447324 (SEQ ID NO:1410), CeresClone:1982544 (SEQ ID NO:1412), CeresAnnot:863218 (SEQ ID NO:1414), CeresAnnot:8733417 (SEQ ID NO:1416), CeresAnnot:8671598 (SEQ ID NO:1418), CeresClone:1004743 (SEQ ID NO:1420), GI:212274719 (SEQ ID NO:1421), GI:678547 (SEQ ID NO:1422), or CeresAnnot:1488034 (SEQ ID NO:1424). In some cases, a functional homolog of SEQ ID NO:1318 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:1318.

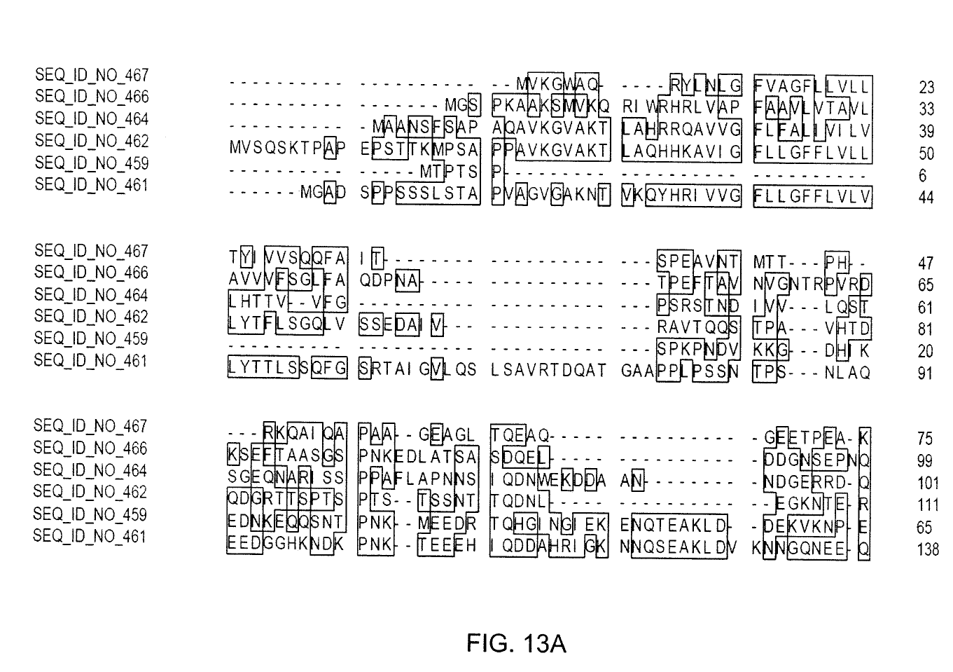

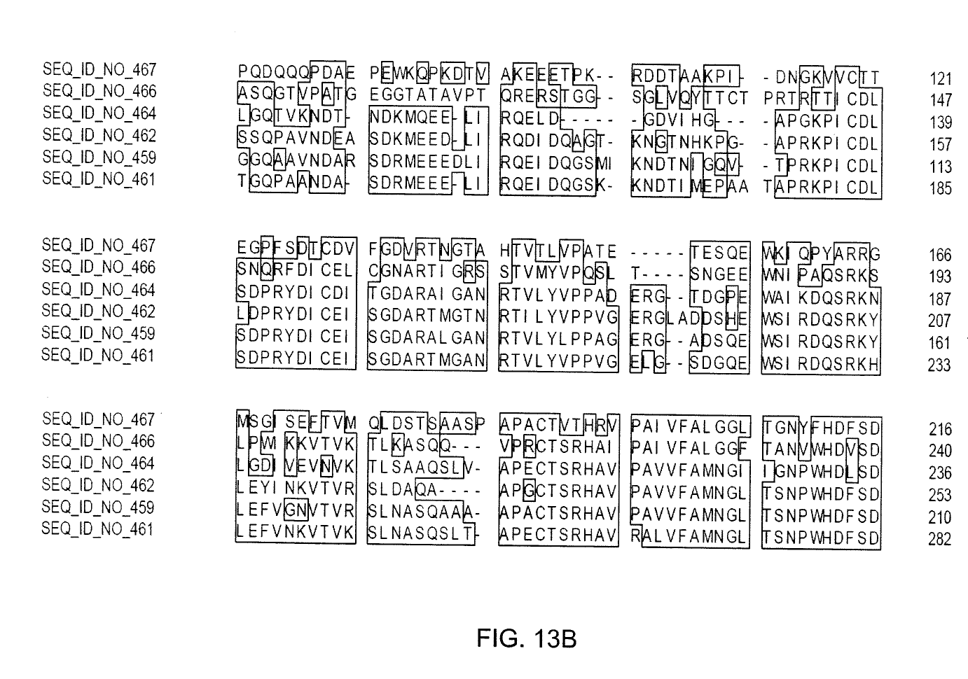

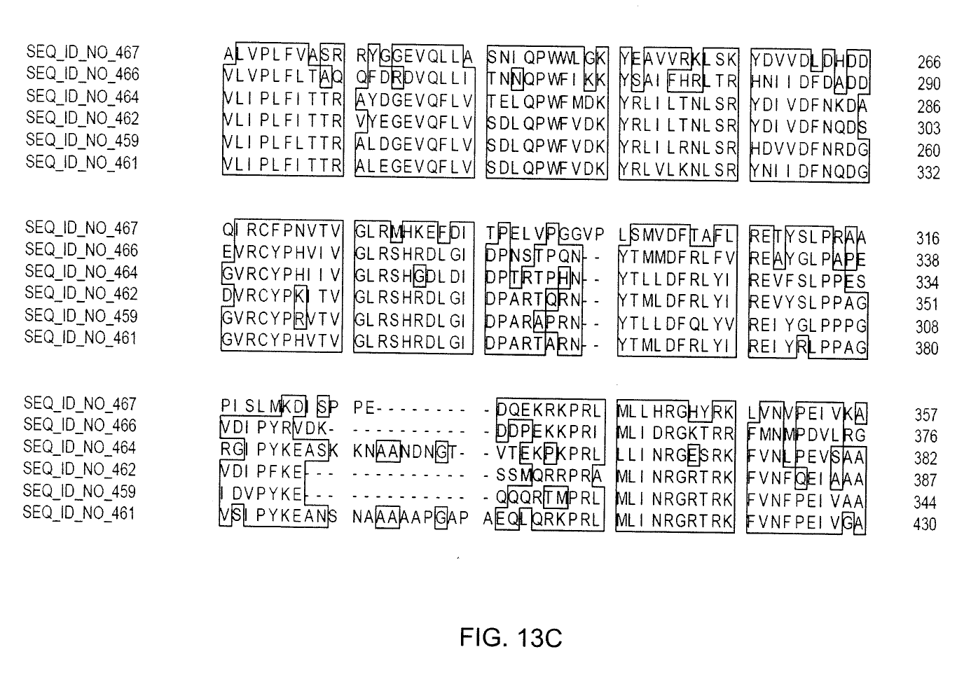

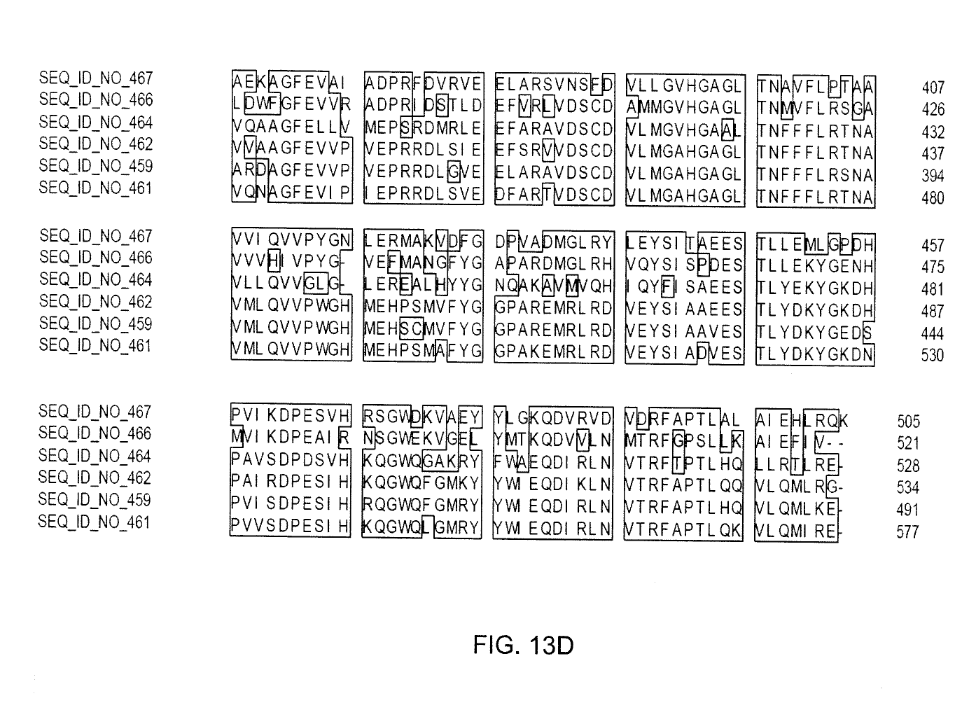

Examples of amino acid sequences of functional homologs of the polypeptide set forth in SEQ ID NO:459 are provided in FIG. 13 and in the Sequence Listing. Such functional homologs include, for example, CeresClone:219838 (SEQ ID NO:461), GI:115468072 (SEQ ID NO:462), CeresClone:819125 (SEQ ID NO:464), CeresAnnot:8678791 (SEQ ID NO:466), GI:55792425 (SEQ ID NO:467), GI:194702014 (SEQ ID NO:468), GI:125563028 (SEQ ID NO:469), GI:115445865 (SEQ ID NO:470), CeresClone:1784280 (SEQ ID NO:472), CeresAnnot:8681869 (SEQ ID NO:474), GI:31296712 (SEQ ID NO:475), CeresAnnot:8659077 (SEQ ID NO:477), CeresAnnot:8659066 (SEQ ID NO:479), GI:194690360 (SEQ ID NO:480), CeresClone:375289 (SEQ ID NO:482), GI:212274959 (SEQ ID NO:483), GI:115434208 (SEQ ID NO:484), GI:56409854 (SEQ ID NO:485), CeresClone:1748522 (SEQ ID NO:487), GI:115457236 (SEQ ID NO:488), GI:116317832 (SEQ ID NO:489), GI:115434210 (SEQ ID NO:490), GI:56409856 (SEQ ID NO:491), GI:125568815 (SEQ ID NO:492), GI:31296713 (SEQ ID NO:493), GI:125524186 (SEQ ID NO:494), GI:55792424 (SEQ ID NO:495), CeresAnnot:8659052 (SEQ ID NO:497), or CeresAnnot:8659059 (SEQ ID NO:499). In some cases, a functional homolog of SEQ ID NO:459 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:459.

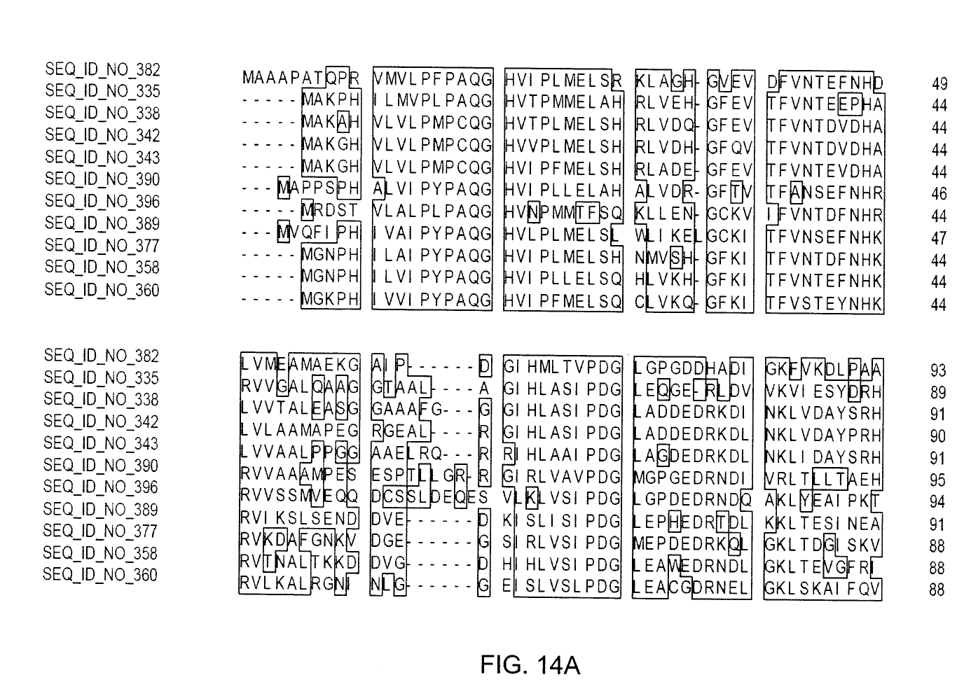

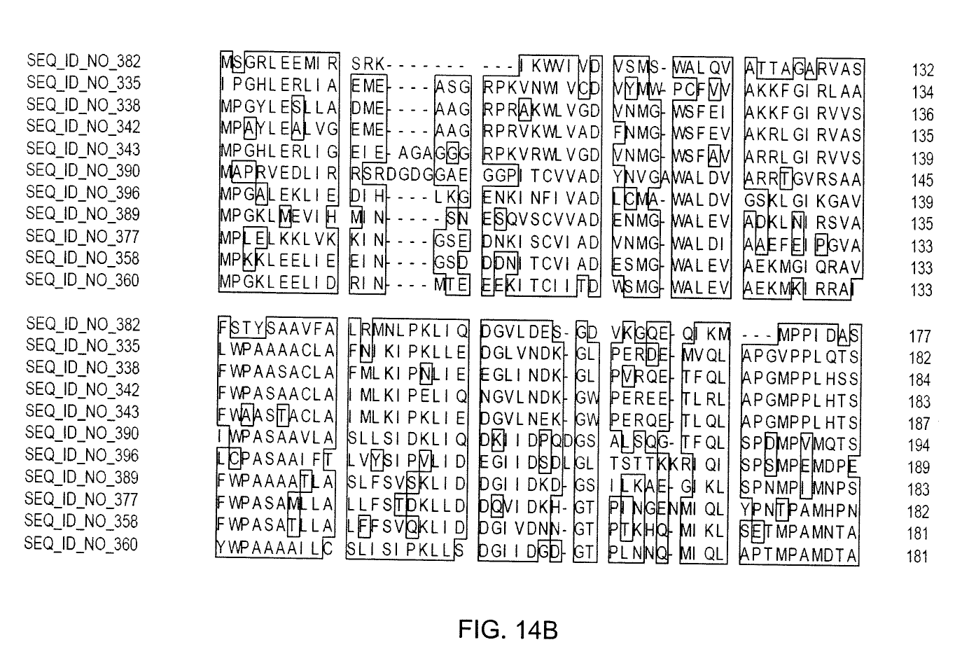

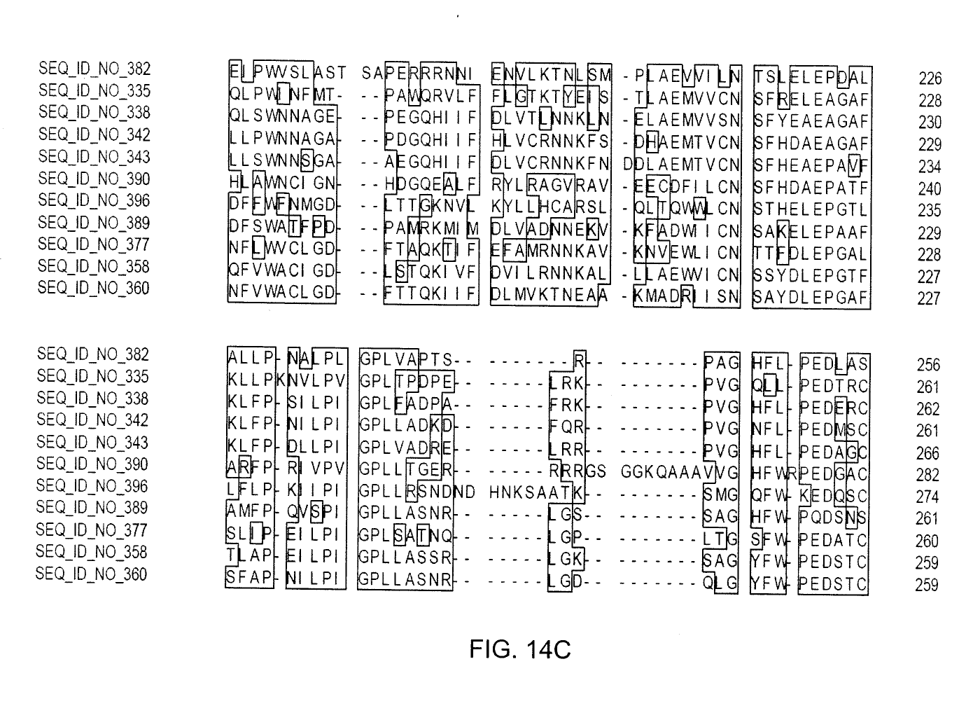

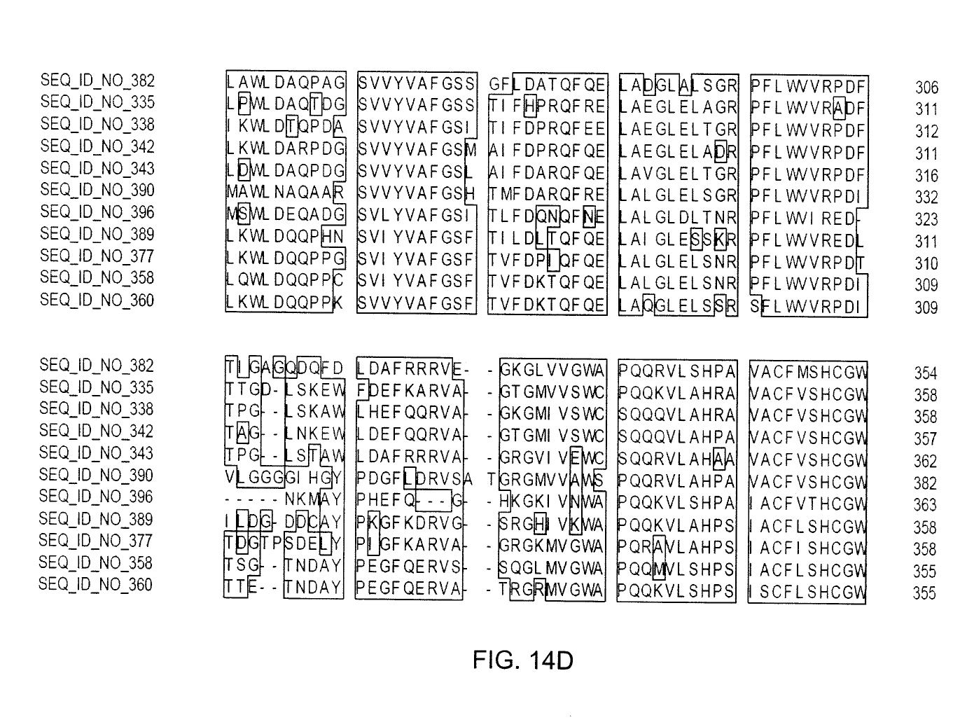

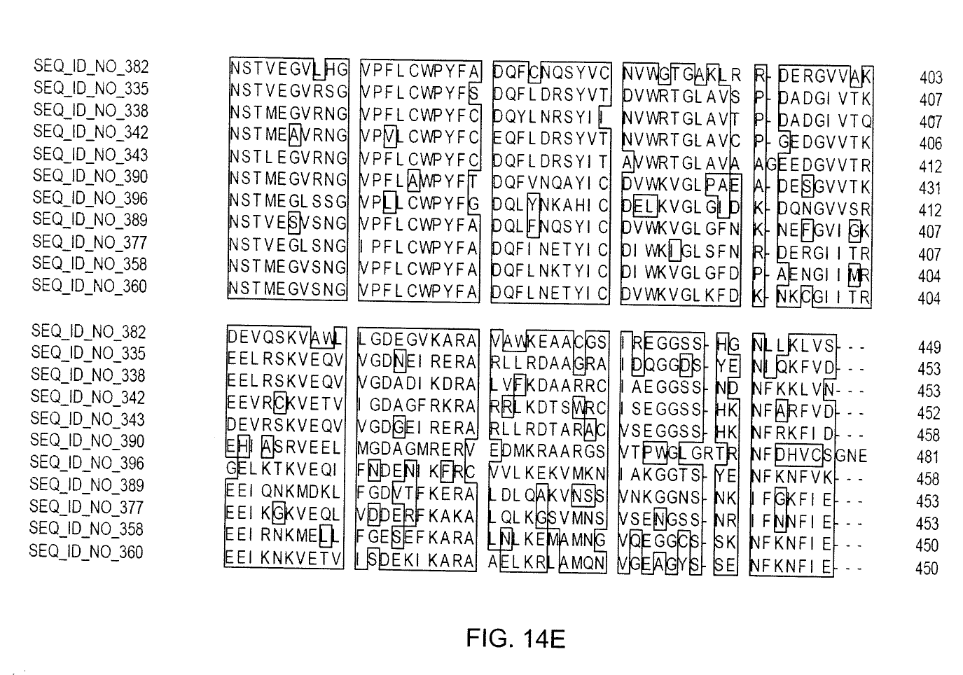

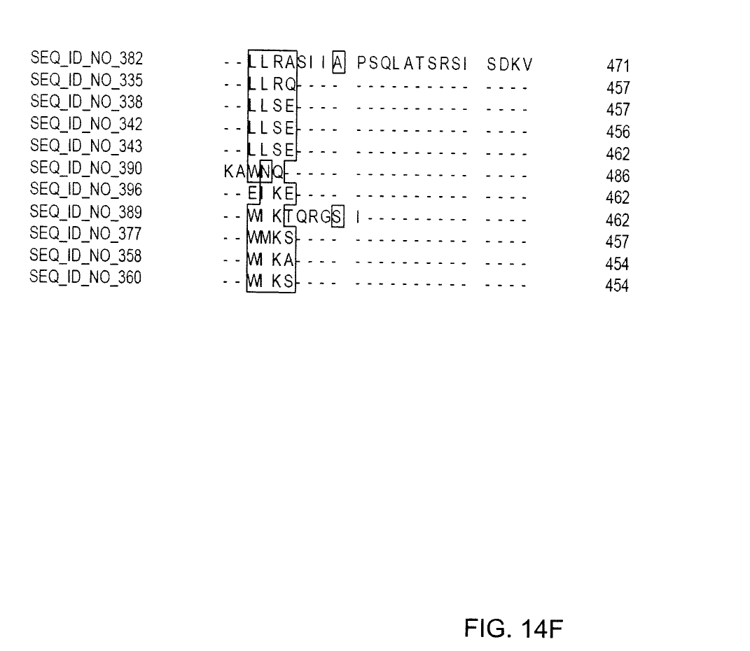

Examples of amino acid sequences of functional homologs of the polypeptide set forth in SEQ ID NO:335 are provided in FIG. 14 and in the Sequence Listing. Such functional homologs include, for example, CeresClone:1988535 (SEQ ID NO:337), GI:21326123 (SEQ ID NO:338), CeresAnnot:8632623 (SEQ ID NO:340), CeresClone:702826 (SEQ ID NO:342), GI:13236659 (SEQ ID NO:343), CeresAnnot:8632621 (SEQ ID NO:345), GI:115455443 (SEQ ID NO:346), GI:125545776 (SEQ ID NO:347), CeresAnnot:8632620 (SEQ ID NO:349), GI:115455447 (SEQ ID NO:350), GI:115455451 (SEQ ID NO:351), CeresAnnot:8632618 (SEQ ID NO:353), GI:13236658 (SEQ ID NO:354), GI:125587975 (SEQ ID NO:355), CeresClone:1776335 (SEQ ID NO:357), GI:157341250 (SEQ ID NO:358), CeresAnnot:1450062 (SEQ ID NO:360), CeresAnnot:8632619 (SEQ ID NO:362), GI:157342353 (SEQ ID NO:363), GI:21326128 (SEQ ID NO:364), GI:157341249 (SEQ ID NO:365), CeresAnnot:1450233 (SEQ ID NO:367), CeresAnnot:1476898 (SEQ ID NO:369), GI:147802898 (SEQ ID NO:370), GI:157342349 (SEQ ID NO:371), GI:157342357 (SEQ ID NO:372), GI:147822731 (SEQ ID NO:373), GI:157341247 (SEQ ID NO:374), GI:157342350 (SEQ ID NO:375), CeresClone:1933155 (SEQ ID NO:377), GI:157341834 (SEQ ID NO:378), GI:157341251 (SEQ ID NO:379), GI:157342355 (SEQ ID NO:380), CeresClone:678597 (SEQ ID NO:382), GI:157342346 (SEQ ID NO:383), GI:147782587 (SEQ ID NO:384), GI:21326126 (SEQ ID NO:385), GI:125531499 (SEQ ID NO:386), GI:22655755 (SEQ ID NO:387), CeresAnnot:8454605 (SEQ ID NO:389), GI:212721816 (SEQ ID NO:390), GI:157342345 (SEQ ID NO:391), CeresAnnot:8456560 (SEQ ID NO:393), CeresAnnot:8632034 (SEQ ID NO:395), GI:10953887 (SEQ ID NO:396), CeresClone:463302 (SEQ ID NO:398), CeresClone:337235 (SEQ ID NO:400), or WO2008034648-136421 (SEQ ID NO:401). In some cases, a functional homolog of SEQ ID NO:335 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:335.

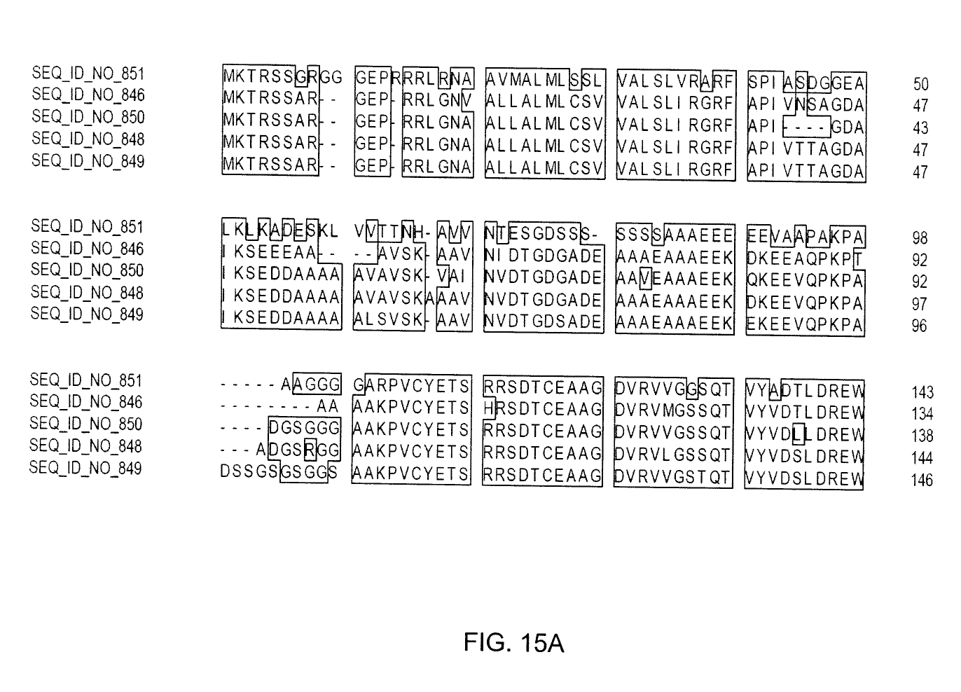

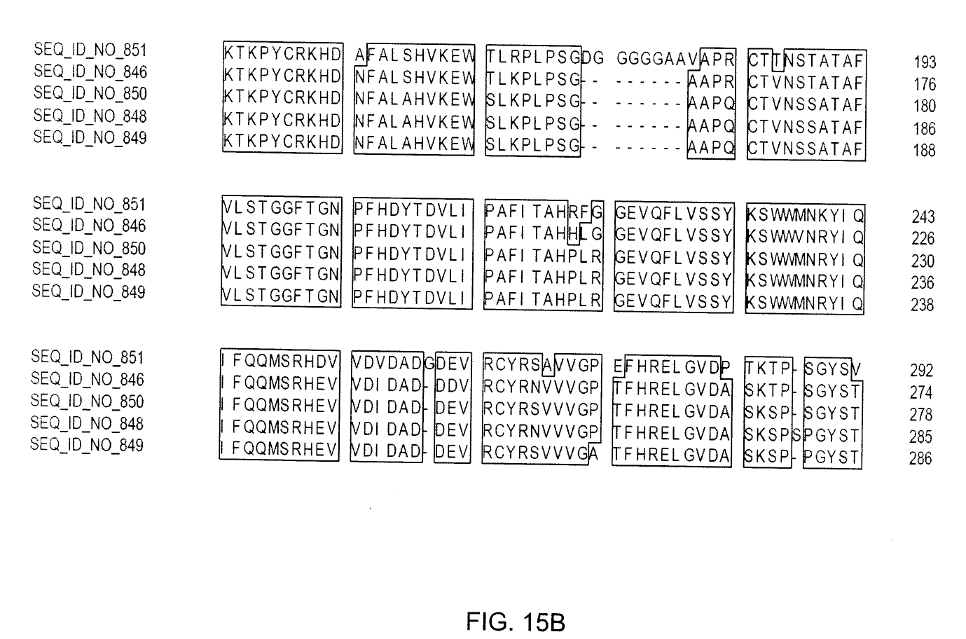

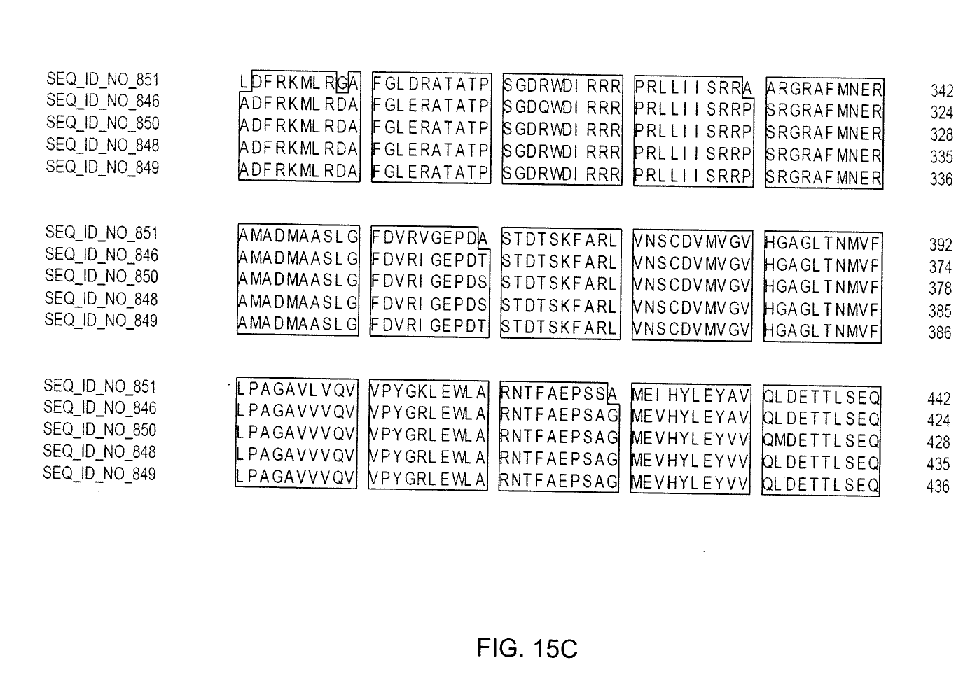

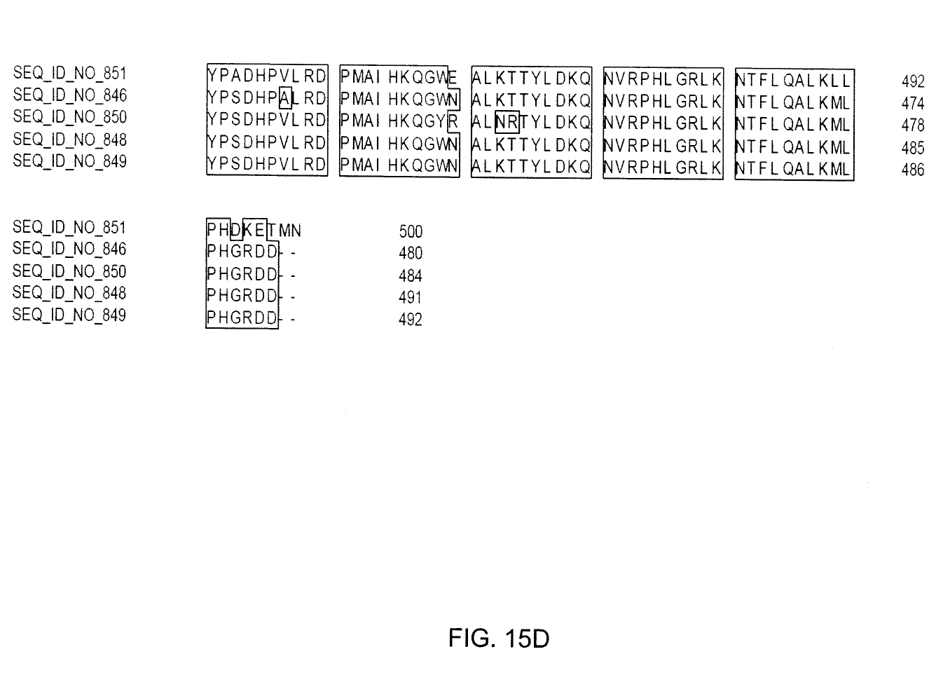

Examples of amino acid sequences of functional homologs of the polypeptide set forth in SEQ ID NO:846 are provided in FIG. 15 and in the Sequence Listing. Such functional homologs include, for example, CeresAnnot:8670072 (SEQ ID NO:848), GI:162460449 (SEQ ID NO:849), GI:56409862 (SEQ ID NO:850), GI:115482674 (SEQ ID NO:851), CeresClone:1770027 (SEQ ID NO:853), CeresClone:100815759 (SEQ ID NO:855), GI:46390169 (SEQ ID NO:856), GI:115445889 (SEQ ID NO:857), GI:125581917 (SEQ ID NO:858), CeresAnnot:8734902 (SEQ ID NO:860), GI:125539258 (SEQ ID NO:861), CeresClone:1726616 (SEQ ID NO:863), GI:115444075 (SEQ ID NO:864), CeresClone:825530 (SEQ ID NO:866), CeresAnnot:8671335 (SEQ ID NO:868), CeresClone:1795292 (SEQ ID NO:870), GI:125580725 (SEQ ID NO:871), CeresAnnot:8744577 (SEQ ID NO:873), GI:115469852 (SEQ ID NO:874), or CeresClone:845464 (SEQ ID NO:876). In some cases, a functional homolog of SEQ ID NO:846 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:846.

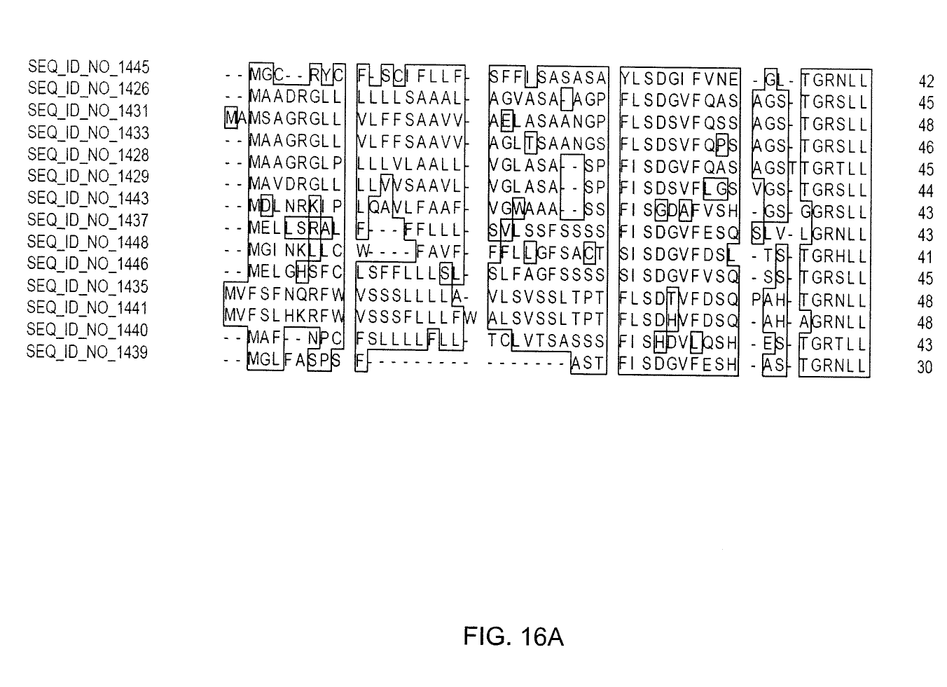

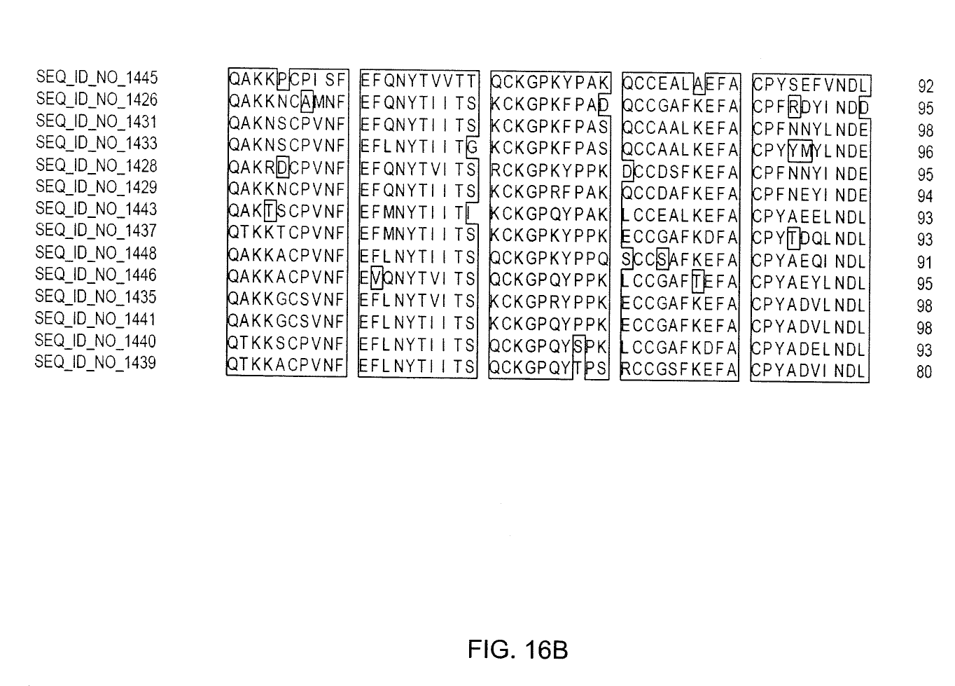

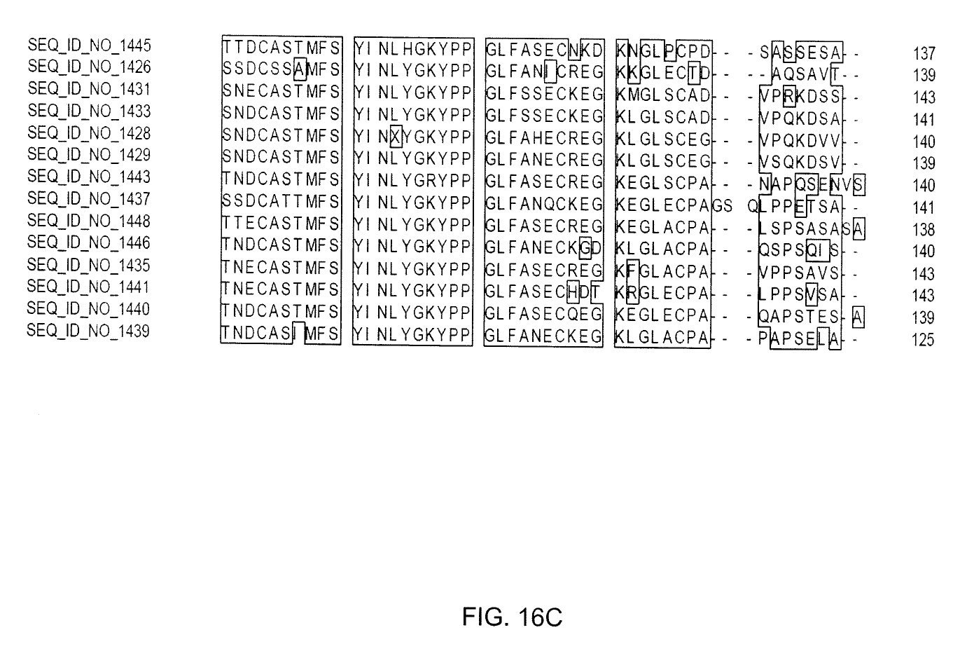

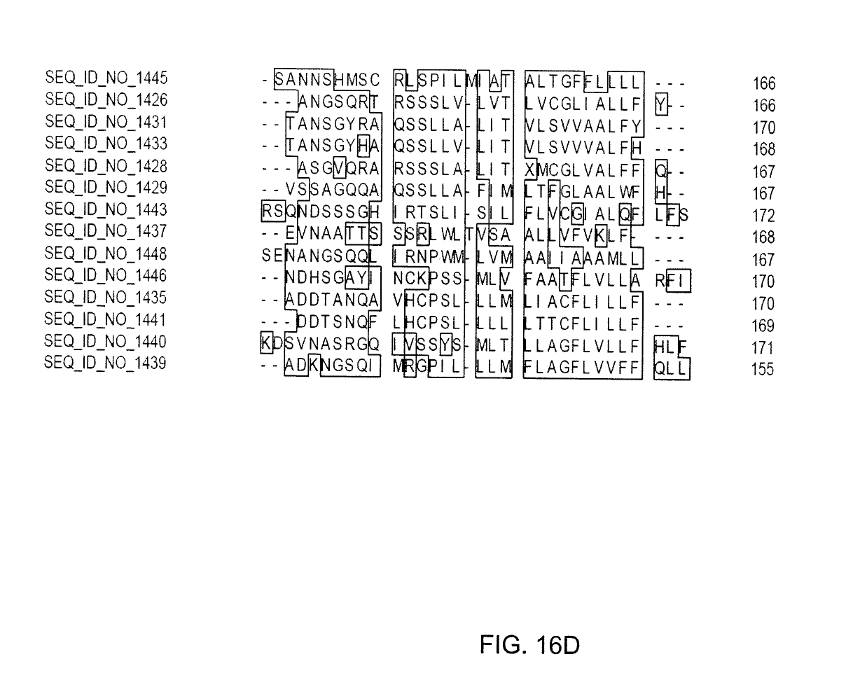

Examples of amino acid sequences of functional homologs of the polypeptide set forth in SEQ ID NO:1426 are provided in FIG. 16 and in the Sequence Listing. Such functional homologs include, for example, CeresClone:729979 (SEQ ID NO:1428), GI:115448365 (SEQ ID NO:1429), CeresClone:1451881 (SEQ ID NO:1431), CeresAnnot:8680955 (SEQ ID NO:1433), CeresClone:1047334 (SEQ ID NO:1435), CeresClone:6841 (SEQ ID NO:1437), CeresAnnot:1495176 (SEQ ID NO:1439), GI:225446345 (SEQ ID NO:1440), GI:3868853 (SEQ ID NO:1441), CeresClone:1727134 (SEQ ID NO:1443), CeresAnnot:8458866 (SEQ ID NO:1445), GI:124484379 (SEQ ID NO:1446), CeresClone:1970015 (SEQ ID NO:1448), CeresClone:1879623 (SEQ ID NO:1450), CeresClone:1994946 (SEQ ID NO:1452), CeresClone:101992420 (SEQ ID NO:1454), CeresClone:1790201 (SEQ ID NO:1456), GI:125540938 (SEQ ID NO:1457), CeresClone:1961617 (SEQ ID NO:1459), CeresClone:236422 (SEQ ID NO:1461), CeresClone:481087 (SEQ ID NO:1463), CeresClone:955288 (SEQ ID NO:1465), CeresClone:628828 (SEQ ID NO:1467), CeresClone:1939564 (SEQ ID NO:1469), CeresClone:100011399 (SEQ ID NO:1471), CeresAnnot:1480568 (SEQ ID NO:1473), CeresClone:1927778 (SEQ ID NO:1475), CeresClone:1446925 (SEQ ID NO:1477), or CeresClone:1727113 (SEQ ID NO:1479). In some cases, a functional homolog of SEQ ID NO:1426 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:1426.

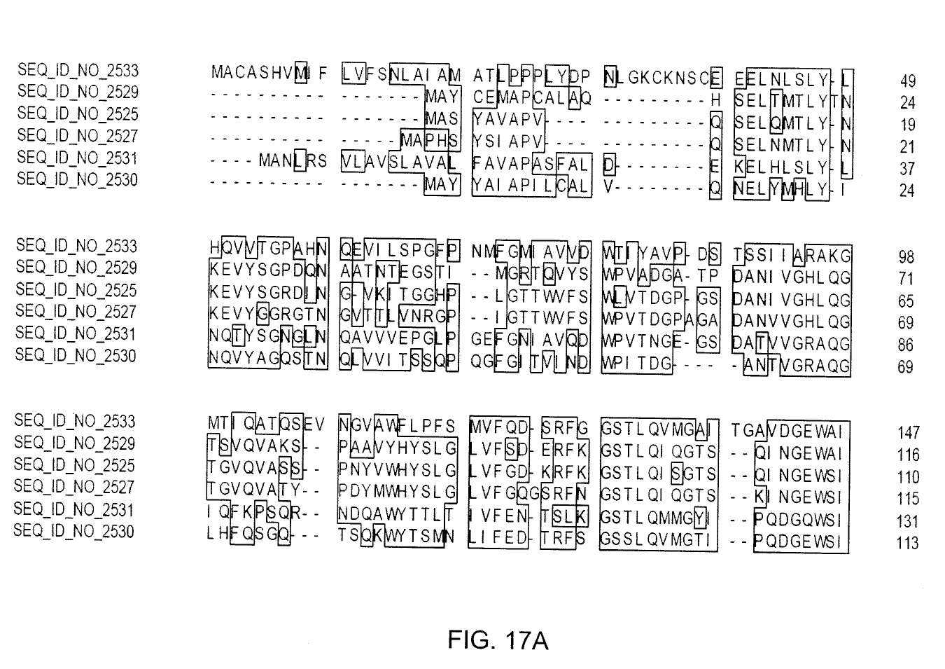

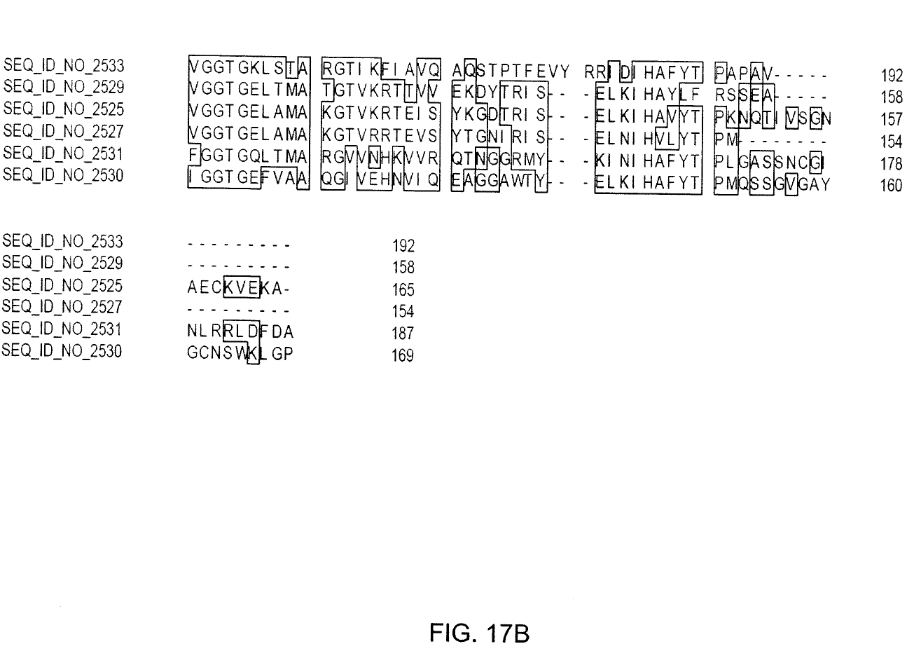

Examples of amino acid sequences of functional homologs of the polypeptide set forth in SEQ ID NO:2525 are provided in FIG. 17 and in the Sequence Listing. Such functional homologs include, for example, CeresClone:100892614 (SEQ ID NO:2527), CeresAnnot:8692947 (SEQ ID NO:2529), GI:53791709 (SEQ ID NO:2530), GI:55416171 (SEQ ID NO:2531), CeresClone:1019755 (SEQ ID NO:2533), CeresClone:1956225 (SEQ ID NO:2535), CeresClone:379196 (SEQ ID NO:2537), CeresClone:275753 (SEQ ID NO:2539), GI:125525841 (SEQ ID NO:2540), GI:37700483 (SEQ ID NO:2541), or GI:42454402 (SEQ ID NO:2542). In some cases, a functional homolog of SEQ ID NO:2525 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2525.

Examples of amino acid sequences of functional homologs of the polypeptide set forth in SEQ ID NO:1272 are provided in FIG. 18 and in the Sequence Listing. Such functional homologs include, for example, CeresAnnot:8726250 (SEQ ID NO:1274), CeresClone:899059 (SEQ ID NO:1276), CeresClone:945132 (SEQ ID NO:1278), GI:115462673 (SEQ ID NO:1279), CeresClone:16400 (SEQ ID NO:1281), CeresClone:1712201 (SEQ ID NO:1283), CeresAnnot:1524669 (SEQ ID NO:1285), CeresAnnot:8672987 (SEQ ID NO:1287), CeresClone:1434951 (SEQ ID NO:1289), CeresClone:299745 (SEQ ID NO:1291), CeresClone:323696 (SEQ ID NO:1293), GI:194695666 (SEQ ID NO:1294), CeresClone:1771257 (SEQ ID NO:1296), GI:115445433 (SEQ ID NO:1297), CeresAnnot:8667876 (SEQ ID NO:1299), GI:115438957 (SEQ ID NO:1300), CeresClone:1100814 (SEQ ID NO:1302), CeresClone:1029710 (SEQ ID NO:1304), CeresClone:969326 (SEQ ID NO:1306), CeresClone:100955392 (SEQ ID NO:1308), GI:225454450 (SEQ ID NO:1309), GI:116779724 (SEQ ID NO:1310), CeresAnnot:1447561 (SEQ ID NO:1312), GI:20149060 (SEQ ID NO:1313), GI:225462683 (SEQ ID NO:1314), or CeresClone:595099 (SEQ ID NO:1316). In some cases, a functional homolog of SEQ ID NO:1272 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:1272.

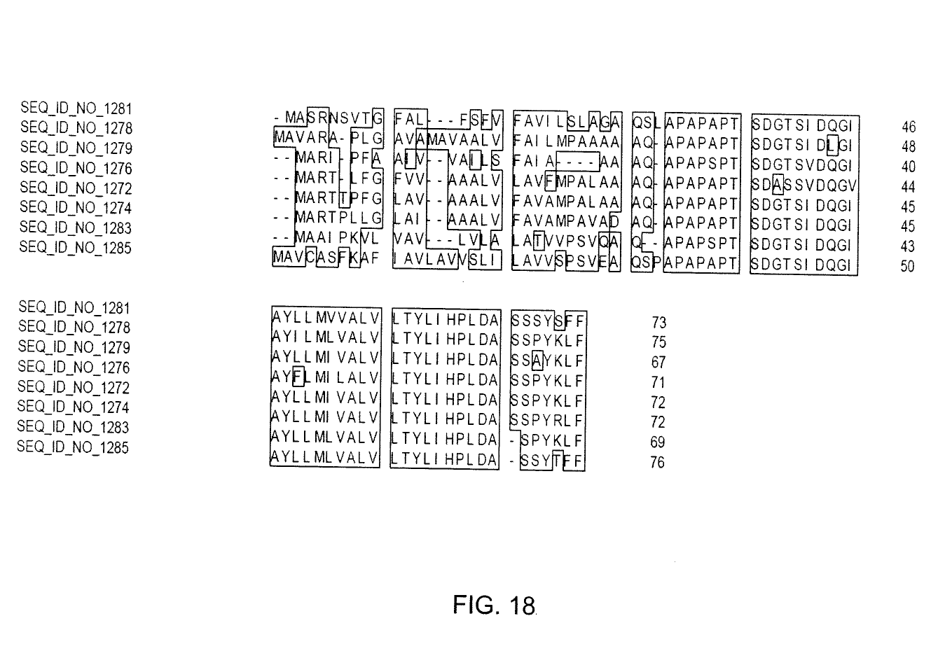

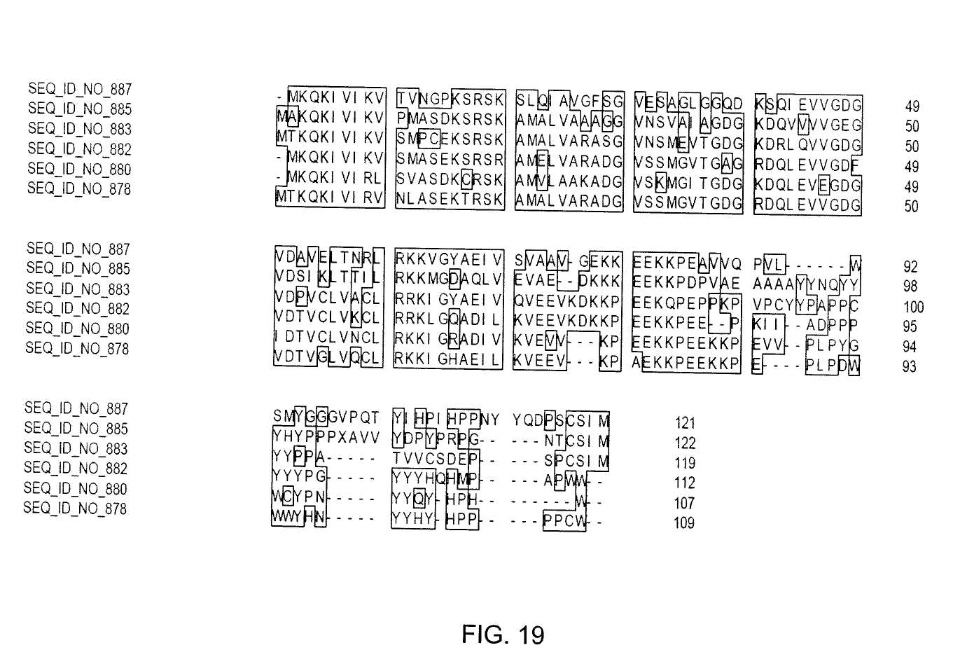

Examples of amino acid sequences of functional homologs of the polypeptide set forth in SEQ ID NO:878 are provided in FIG. 19 and in the Sequence Listing. Such functional homologs include, for example, CeresAnnot:8701398 (SEQ ID NO:880), CeresClone:2030878 (SEQ ID NO:882), GI:21741986 (SEQ ID NO:883), CeresClone:488555 (SEQ ID NO:885), CeresAnnot:1472210 (SEQ ID NO:887), CeresClone:1778664 (SEQ ID NO:889), GI:115458882 (SEQ ID NO:890), CeresAnnot:8701404 (SEQ ID NO:892), GI:115458830 (SEQ ID NO:893), CeresAnnot:8701387 (SEQ ID NO:895), GI:116310418 (SEQ ID NO:896), GI:46806258 (SEQ ID NO:897), CeresAnnot:8679943 (SEQ ID NO:899), CeresAnnot:8701391 (SEQ ID NO:901), GI:46806257 (SEQ ID NO:902), GI:125540058 (SEQ ID NO:903), CeresClone:1018979 (SEQ ID NO:905), CeresClone:1725423 (SEQ ID NO:907), GI:115446965 (SEQ ID NO:908), GI:125540059 (SEQ ID NO:909), GI:115458886 (SEQ ID NO:910), CeresClone:1955791 (SEQ ID NO:912), CeresClone:2032166 (SEQ ID NO:914), GI:125540060 (SEQ ID NO:915), GI:46806261 (SEQ ID NO:916), GI:115458884 (SEQ ID NO:917), CeresClone:100178733 (SEQ ID NO:919), CeresClone:351547 (SEQ ID NO:921), CeresClone:1906874 (SEQ ID NO:923), CeresClone:1885857 (SEQ ID NO:925), GI:38606532 (SEQ ID NO:926), CeresClone:1771513 (SEQ ID NO:928), CeresClone:1950928 (SEQ ID NO:930), CeresClone:273420 (SEQ ID NO:932), CeresAnnot:8701399 (SEQ ID NO:934), GI:194700536 (SEQ ID NO:935), GI:125540061 (SEQ ID NO:936), GI:115446971 (SEQ ID NO:937), CeresClone:1802499 (SEQ ID NO:939), CeresClone:1839543 (SEQ ID NO:941), CeresClone:1850157 (SEQ ID NO:943), or CeresClone:1471240 (SEQ ID NO:945). In some cases, a functional homolog of SEQ ID NO:878 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:878.

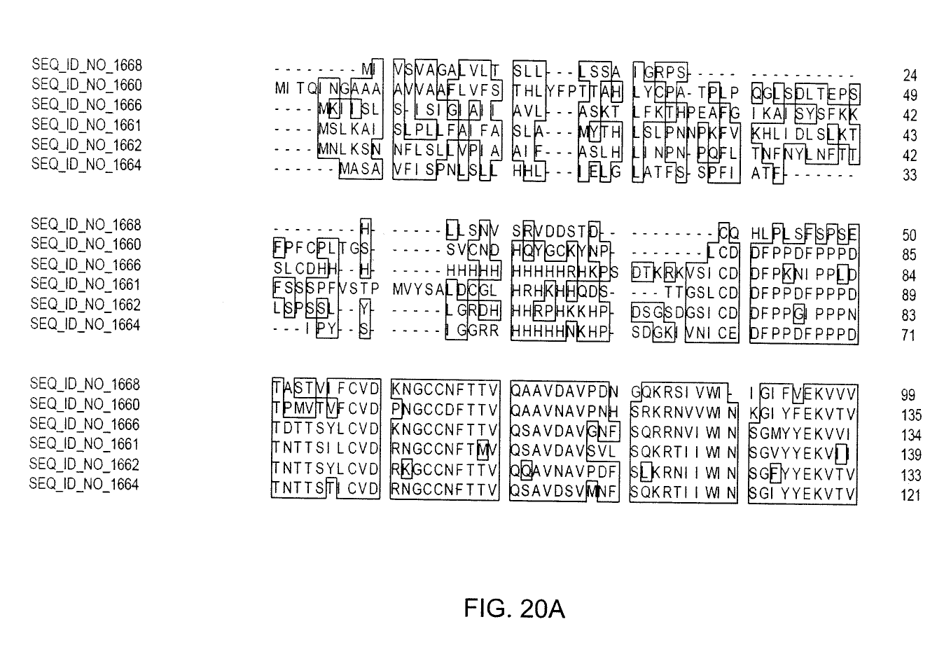

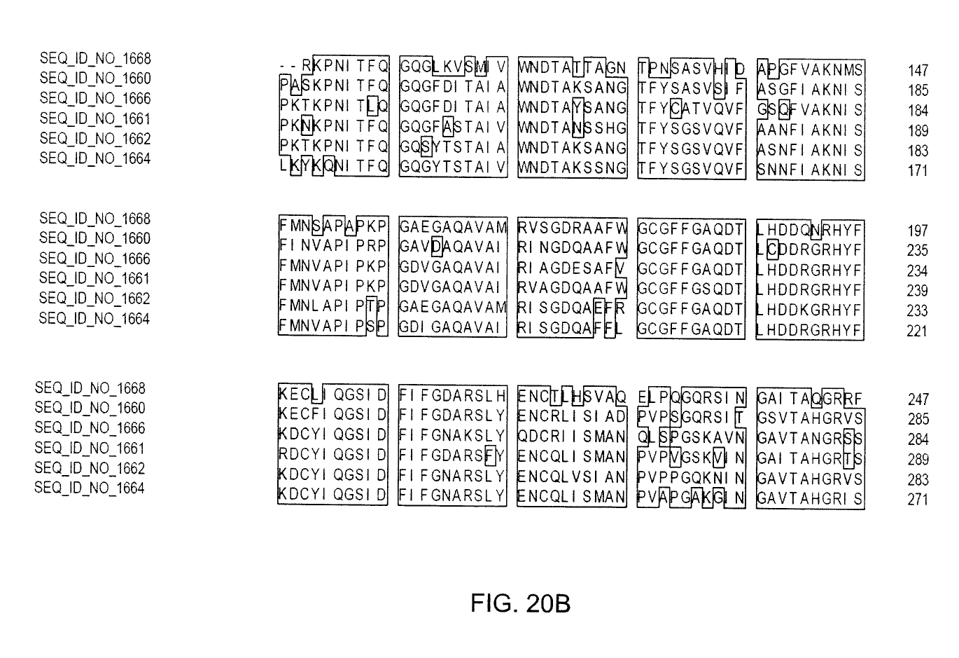

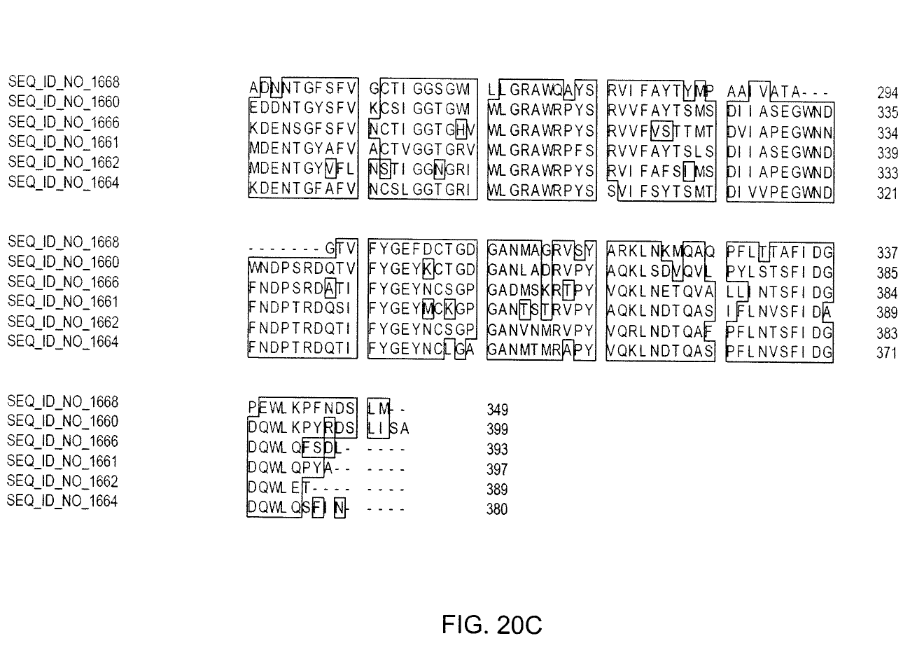

Examples of amino acid sequences of functional homologs of the polypeptide set forth in SEQ ID NO:1660 are provided in FIG. 20 and in the Sequence Listing. Such functional homologs include, for example, GI:157355547 (SEQ ID NO:1661), GI:124360659 (SEQ ID NO:1662), CeresAnnot:1527994 (SEQ ID NO:1664), CeresClone:1150649 (SEQ ID NO:1666), CeresAnnot:8681260 (SEQ ID NO:1668), GI:15220471 (SEQ ID NO:1669), GI:2388565 (SEQ ID NO:1670), GI:147825336 (SEQ ID NO:1671), GI:157356769 (SEQ ID NO:1672), CeresAnnot:1485042 (SEQ ID NO:1674), CeresAnnot:861154 (SEQ ID NO:1676), GI:27754550 (SEQ ID NO:1677), CeresAnnot:8647624 (SEQ ID NO:1679), GI:115480795 (SEQ ID NO:1680), GI:15241163 (SEQ ID NO:1681), GI:157342726 (SEQ ID NO:1682), GI:147776052 (SEQ ID NO:1683), GI:168034634 (SEQ ID NO:1684), CeresAnnot:1446775 (SEQ ID NO:1686), CeresClone:1926613 (SEQ ID NO:1688), GI:115464955 (SEQ ID NO:1689), CeresClone:1640838 (SEQ ID NO:1691), CeresClone:1829495 (SEQ ID NO:1693), CeresAnnot:8668633 (SEQ ID NO:1695), CeresClone:797976 (SEQ ID NO:1697), CeresClone:853161 (SEQ ID NO:1699), GI:168033534 (SEQ ID NO:1700), GI:115439873 (SEQ ID NO:1701), GI:116794391 (SEQ ID NO:1702), CeresClone:39717 (SEQ ID NO:1704), GI:15238111 (SEQ ID NO:1705), GI:157327785 (SEQ ID NO:1706), GI:38345478 (SEQ ID NO:1707), GI:9758778 (SEQ ID NO:1708), CeresClone:560813 (SEQ ID NO:1710), GI:89257516 (SEQ ID NO:1711), GI:168033536 (SEQ ID NO:1712), GI:167999009 (SEQ ID NO:1713), GI:168051768 (SEQ ID NO:1714), GI:89257486 (SEQ ID NO:1715), CeresAnnot:1484806 (SEQ ID NO:1717), CeresAnnot:8702233 (SEQ ID NO:1719), CeresAnnot:8463390 (SEQ ID NO:1721), CeresAnnot:1480738 (SEQ ID NO:1723), GI:168023639 (SEQ ID NO:1724), or GI:157342478 (SEQ ID NO:1725). In some cases, a functional homolog of SEQ ID NO:1660 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:1660.

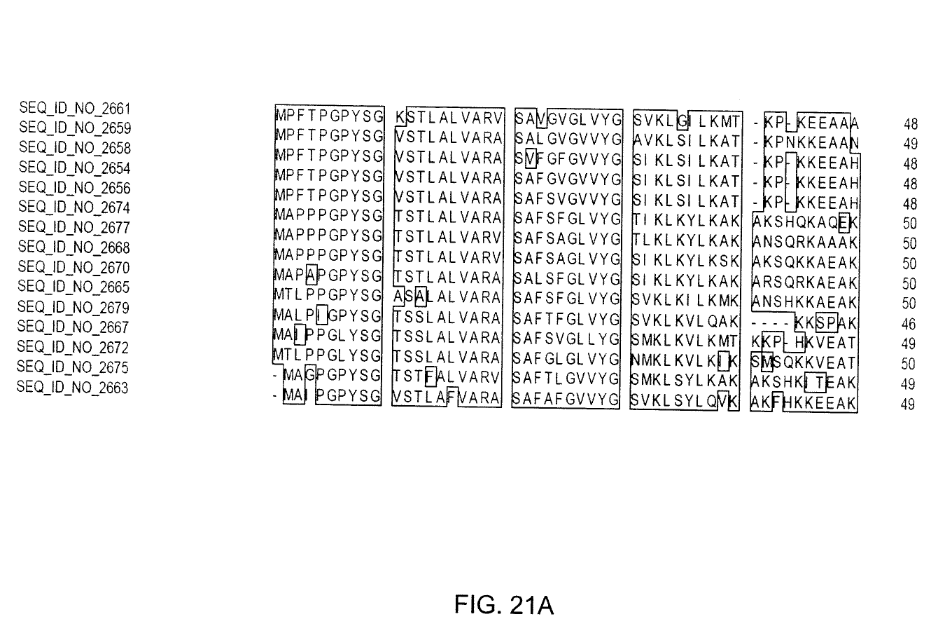

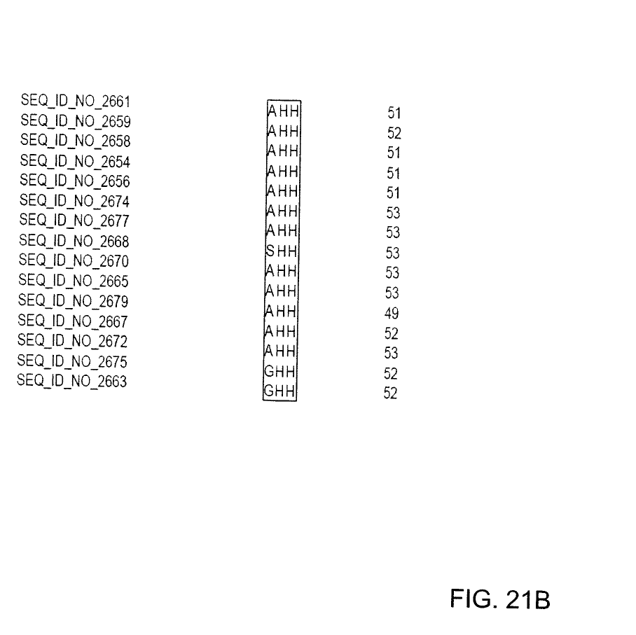

Examples of amino acid sequences of functional homologs of the polypeptide set forth in SEQ ID NO:2654 are provided in FIG. 21 and in the Sequence Listing. Such functional homologs include, for example, CeresAnnot:8701724 (SEQ ID NO:2656), CeresClone:1531656 (SEQ ID NO:2658), GI:115456900 (SEQ ID NO:2659), CeresClone:869980 (SEQ ID NO:2661), CeresClone:1739862 (SEQ ID NO:2663), CeresAnnot:1452831 (SEQ ID NO:2665), CeresClone:1123182 (SEQ ID NO:2667), GI:225427800 (SEQ ID NO:2668), CeresClone:100011527 (SEQ ID NO:2670), CeresClone:1347664 (SEQ ID NO:2672), CeresClone:679875 (SEQ ID NO:2674), GI:116780182 (SEQ ID NO:2675), CeresClone:1606594 (SEQ ID NO:2677), CeresClone:1628000 (SEQ ID NO:2679), CeresClone:1710212 (SEQ ID NO:2681), CeresClone:1649408 (SEQ ID NO:2683), CeresClone:1964117 (SEQ ID NO:2685), CeresClone:660530 (SEQ ID NO:2687), CeresClone:1330898 (SEQ ID NO:2689), CeresClone:1751352 (SEQ ID NO:2691), CeresClone:1068004 (SEQ ID NO:2693), CeresAnnot:1442596 (SEQ ID NO:2695), CeresClone:1898903 (SEQ ID NO:2697), CeresClone:266374 (SEQ ID NO:2699), CeresClone:120248 (SEQ ID NO:2701), CeresAnnot:1513988 (SEQ ID NO:2703), GI:118481824 (SEQ ID NO:2704), or CeresClone:872136 (SEQ ID NO:2706). In some cases, a functional homolog of SEQ ID NO:2654 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2654.

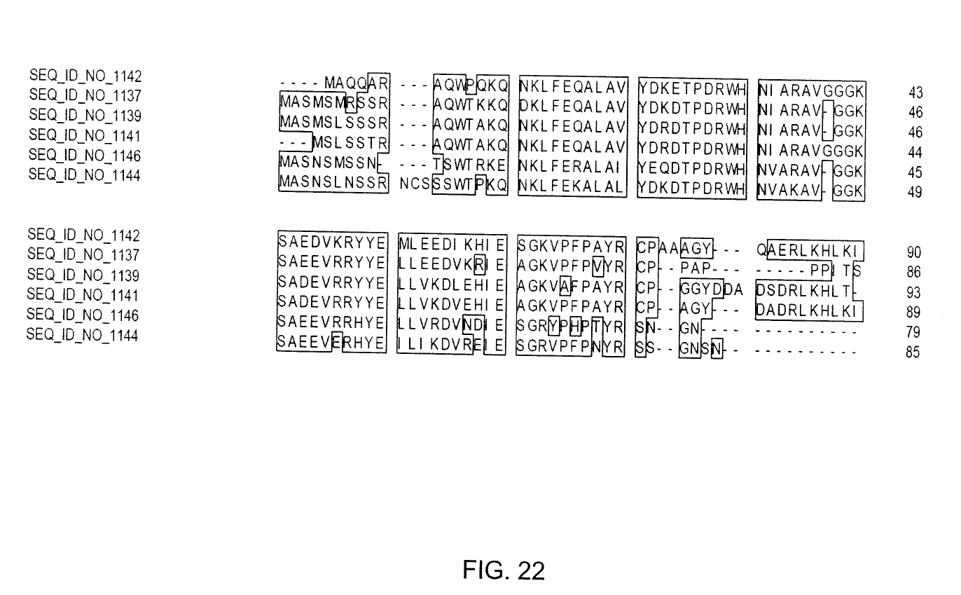

Examples of amino acid sequences of functional homologs of the polypeptide set forth in SEQ ID NO:1137 are provided in FIG. 22 and in the Sequence Listing. Such functional homologs include, for example, CeresClone:331755 (SEQ ID NO:1139), CeresAnnot:8667653 (SEQ ID NO:1141), GI:115465643 (SEQ ID NO:1142), CeresAnnot:1487827 (SEQ ID NO:1144), CeresClone:1069222 (SEQ ID NO:1146), CeresClone:1722230 (SEQ ID NO:1148), CeresClone:1763593 (SEQ ID NO:1150), CeresClone:1910072 (SEQ ID NO:1152), CeresClone:1761808 (SEQ ID NO:1154), CeresAnnot:8734209 (SEQ ID NO:1156), GI:125526974 (SEQ ID NO:1157), GI:115438765 (SEQ ID NO:1158), CeresClone:1523182 (SEQ ID NO:1160), CeresAnnot:1450327 (SEQ ID NO:1162), CeresClone:143475 (SEQ ID NO:1164), CeresAnnot:1514100 (SEQ ID NO:1166), CeresAnnot:1460832 (SEQ ID NO:1168), GI:147767321 (SEQ ID NO:1169), CeresClone:1723374 (SEQ ID NO:1171), CeresAnnot:1510450 (SEQ ID NO:1173), CeresAnnot:1457249 (SEQ ID NO:1175), GI:157335318 (SEQ ID NO:1176), GI:124360540 (SEQ ID NO:1177), CeresClone:100002959 (SEQ ID NO:1179), GI:147784500 (SEQ ID NO:1180), GI:157340812 (SEQ ID NO:1181), CeresClone:477814 (SEQ ID NO:1183), CeresClone:1785685 (SEQ ID NO:1185), CeresClone:1653552 (SEQ ID NO:1187), GI:110931736 (SEQ ID NO:1188), CeresClone:638126 (SEQ ID NO:1190), CeresAnnot:838426 (SEQ ID NO:1192), GI:145327247 (SEQ ID NO:1193), GI:124264312 (SEQ ID NO:1194), CeresClone:1847251 (SEQ ID NO:1196), CeresAnnot:543794 (SEQ ID NO:1198), CeresAnnot:850366 (SEQ ID NO:1200), GI:145356202 (SEQ ID NO:1201), CeresAnnot:8734211 (SEQ ID NO:1203), CeresClone:857342 (SEQ ID NO:1205), CeresAnnot:827713 (SEQ ID NO:1207), CeresClone:1113584 (SEQ ID NO:1209), GI:118137433 (SEQ ID NO:1210), CeresAnnot:1460824 (SEQ ID NO:1212), CeresAnnot:1503394 (SEQ ID NO:1214), CeresClone:1916884 (SEQ ID NO:1216), CeresClone:100068619 (SEQ ID NO:1218), GI:5091605 (SEQ ID NO:1219), CeresClone:1897493 (SEQ ID NO:1221), GI:125553458 (SEQ ID NO:1222), CeresAnnot:1495620 (SEQ ID NO:1224), CeresAnnot:1442707 (SEQ ID NO:1226), CeresAnnot:8461532 (SEQ ID NO:1228), CeresClone:100067803 (SEQ ID NO:1230), CeresAnnot:1679467 (SEQ ID NO:1232), CeresClone:41695 (SEQ ID NO:1234), GI:112292440 (SEQ ID NO:1235), GI:116830269 (SEQ ID NO:1236), CeresClone:1914387 (SEQ ID NO:1238), CeresAnnot:1445878 (SEQ ID NO:1240), GI:7981380 (SEQ ID NO:1241), CeresClone:1927753 (SEQ ID NO:1243), CeresAnnot:8456508 (SEQ ID NO:1245), CeresClone:1086604 (SEQ ID NO:1247), CeresClone:1787372 (SEQ ID NO:1249), CeresClone:2034321 (SEQ ID NO: 1251), GI:112292438 (SEQ ID NO:1252), GI:118489684 (SEQ ID NO:1253), CeresAnnot:8667941 (SEQ ID NO:1255), CeresClone:100965215 (SEQ ID NO:1257), CeresAnnot:8461540 (SEQ ID NO:1259), CeresAnnot:1457231 (SEQ ID NO:1261), CeresAnnot:1457230 (SEQ ID NO:1263), CeresAnnot:1510435 (SEQ ID NO:1265), GI:125553354 (SEQ ID NO:1266), CeresClone:343607 (SEQ ID NO:1268), or CeresClone:100865050 (SEQ ID NO:1270). In some cases, a functional homolog of SEQ ID NO:1137 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:1137.

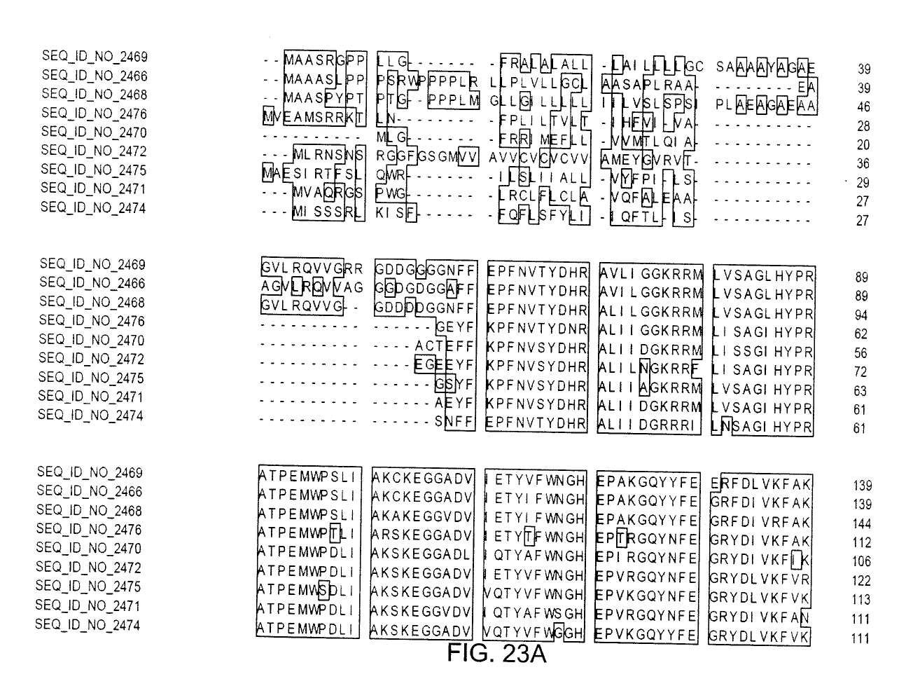

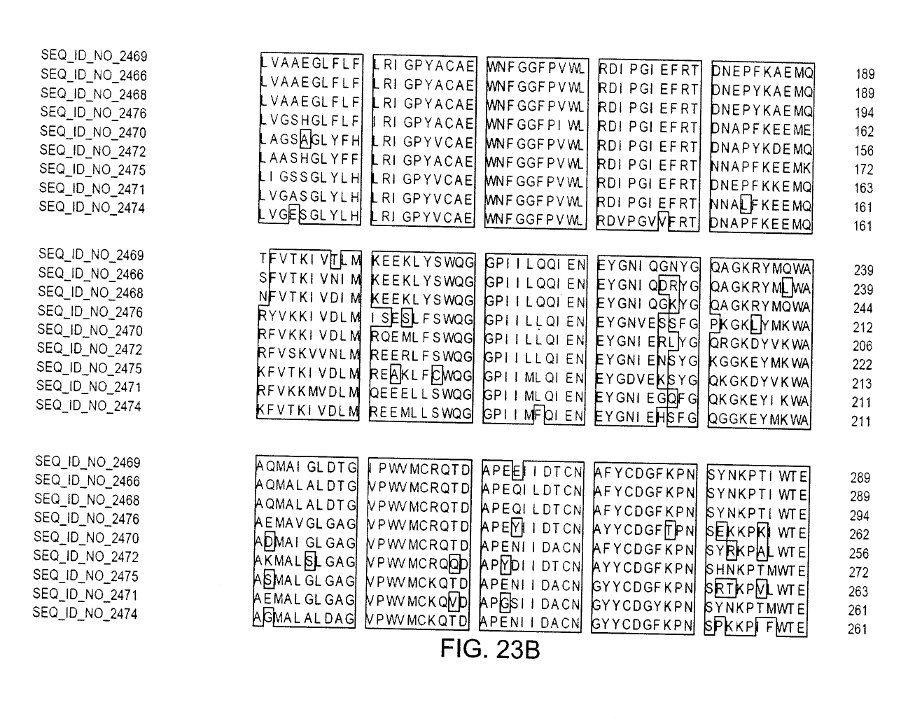

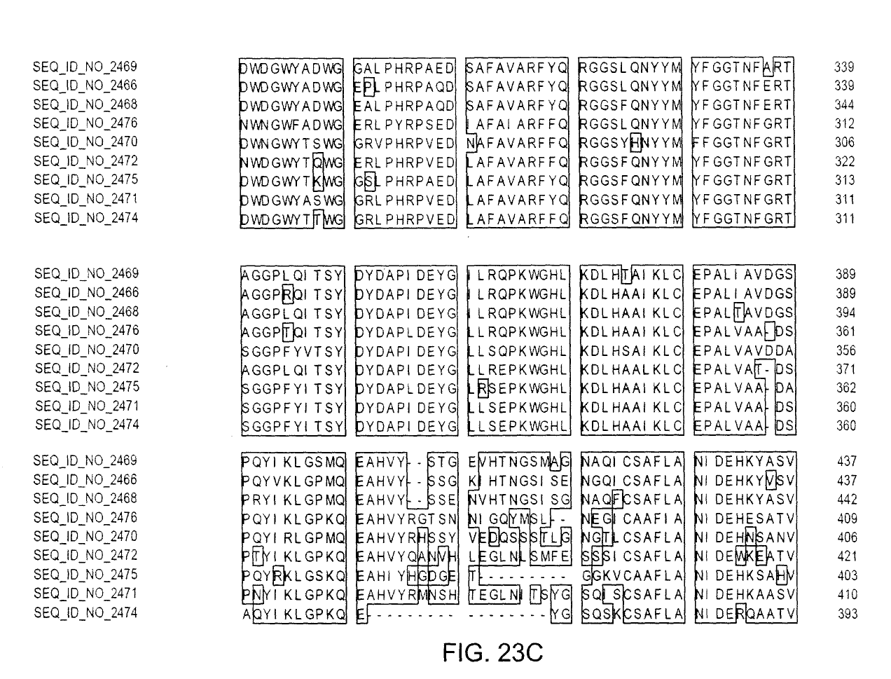

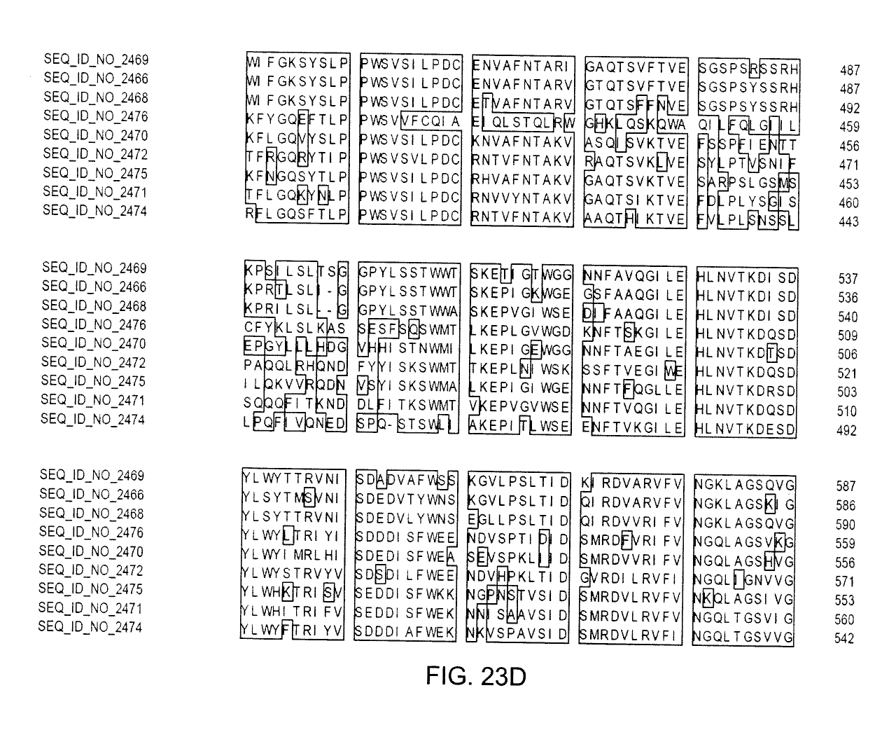

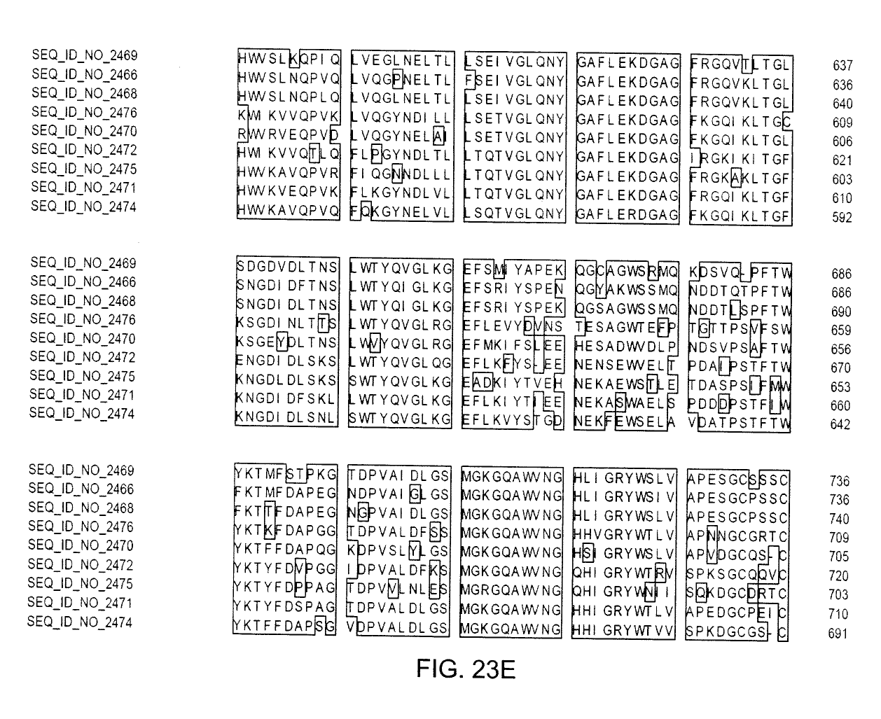

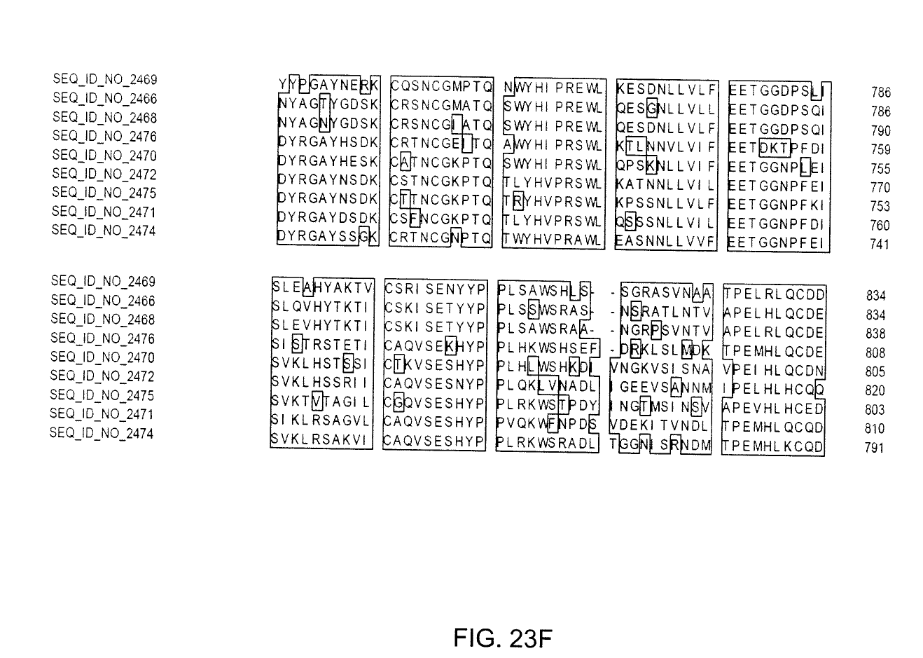

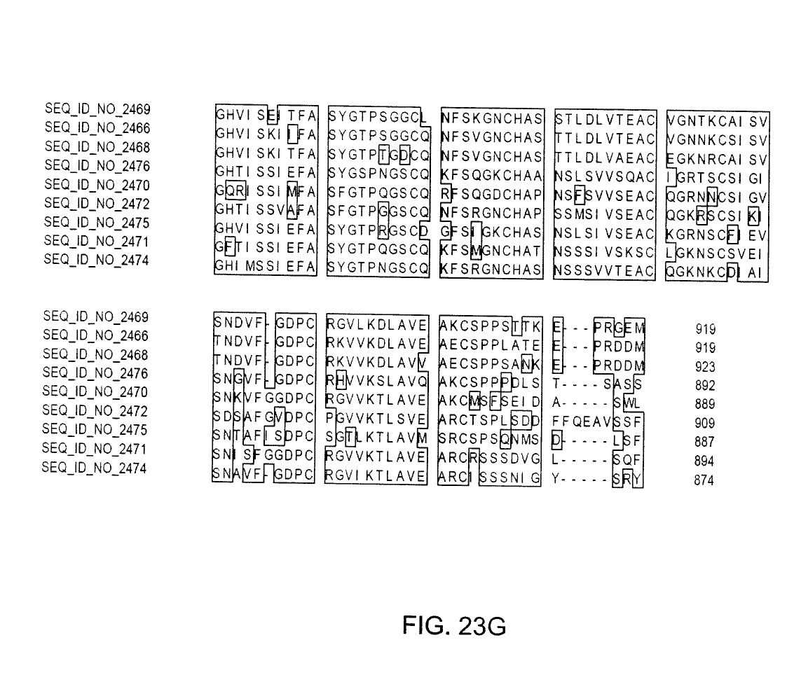

Examples of amino acid sequences of functional homologs of the polypeptide set forth in SEQ ID NO:2466 are provided in FIG. 23 and in the Sequence Listing. Such functional homologs include, for example, CeresAnnot:8715706 (SEQ ID NO:2468), GI:115488372 (SEQ ID NO:2469), GI:114217393 (SEQ ID NO:2470), GI:84579373 (SEQ ID NO:2471), GI:34148077 (SEQ ID NO:2472), CeresAnnot:1543402 (SEQ ID NO:2474), GI:18403090 (SEQ ID NO:2475), GI:7939617 (SEQ ID NO:2476), GI:108862584 (SEQ ID NO:2477), GI:61162194 (SEQ ID NO:2478), CeresClone:1152402 (SEQ ID NO:2480), CeresAnnot:1490015 (SEQ ID NO:2482), GI:168045621 (SEQ ID NO:2483), GI:116787095 (SEQ ID NO:2484), GI:6686888 (SEQ ID NO:2485), GI:56201401 (SEQ ID NO:2486), GI:4510395 (SEQ ID NO:2487), GI:115451981 (SEQ ID NO:2488), GI:125543160 (SEQ ID NO:2489), GI:14970841 (SEQ ID NO:2490), GI:125583741 (SEQ ID NO:2491), CeresAnnot:8642086 (SEQ ID NO:2493), GI:157337481 (SEQ ID NO:2494), GI:61162203 (SEQ ID NO:2495), GI:168008096 (SEQ ID NO:2496), GI:7939619 (SEQ ID NO:2497), GI:183238710 (SEQ ID NO:2498), GI:7939623 (SEQ ID NO:2499), GI:114217395 (SEQ ID NO:2500), CeresAnnot:1515371 (SEQ ID NO:2502), GI:157313304 (SEQ ID NO:2503), CeresAnnot:1462072 (SEQ ID NO:2505), GI:1352077 (SEQ ID NO:2506), GI:108707233 (SEQ ID NO:2507), CeresClone:1860044 (SEQ ID NO:2509), GI:15231354 (SEQ ID NO:2510), GI:20260596 (SEQ ID NO:2511), GI:14970839 (SEQ ID NO:2512), GI:157340015 (SEQ ID NO:2513), GI:118488890 (SEQ ID NO:2514), CeresAnnot:1540596 (SEQ ID NO:2516), GI:61162201 (SEQ ID NO:2517), GI:57232107 (SEQ ID NO:2518), GI:1168654 (SEQ ID NO:2519), CeresAnnot:1543613 (SEQ ID NO:2521), GI:115437888 (SEQ ID NO:2522), or GI:124360385 (SEQ ID NO:2523). In some cases, a functional homolog of SEQ ID NO:2466 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2466.

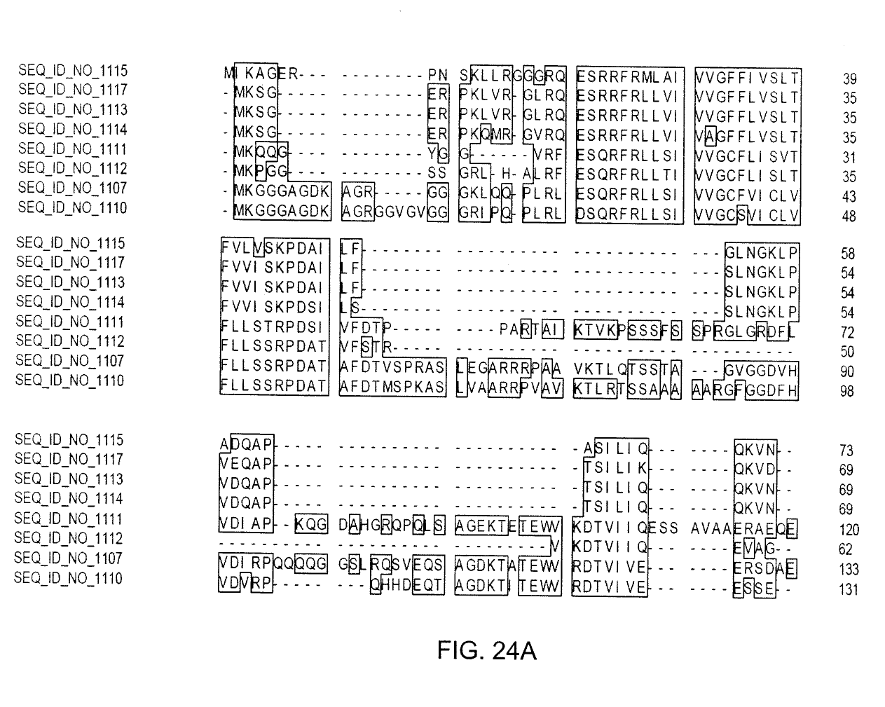

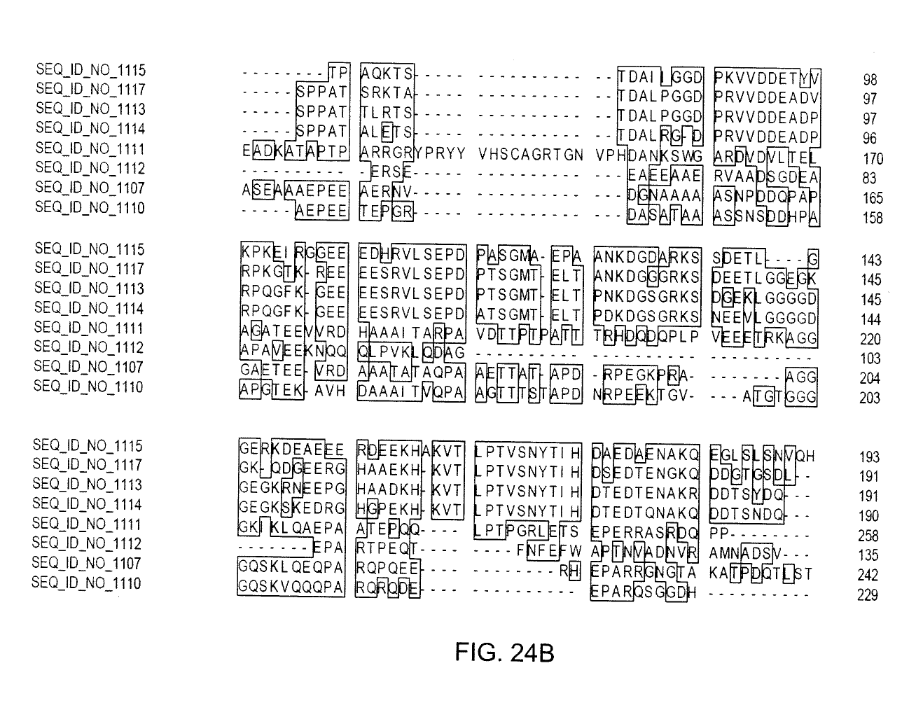

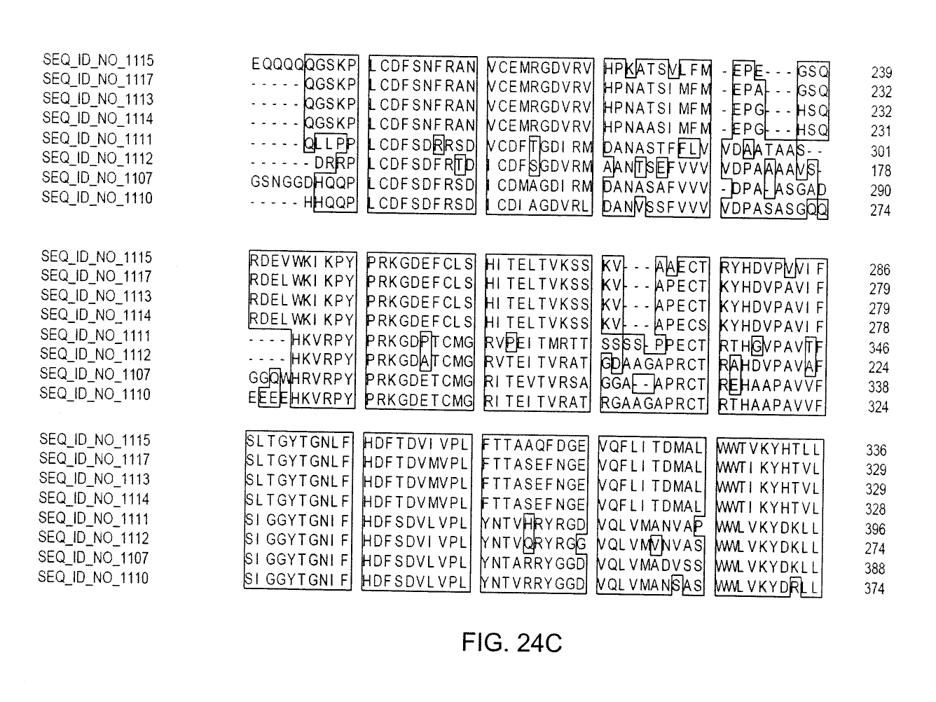

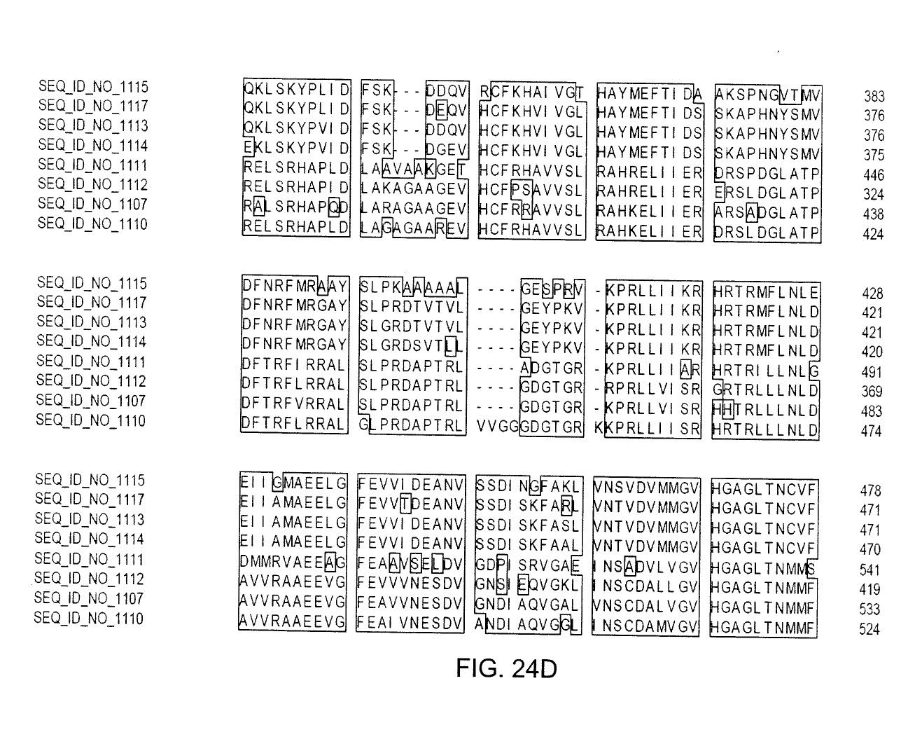

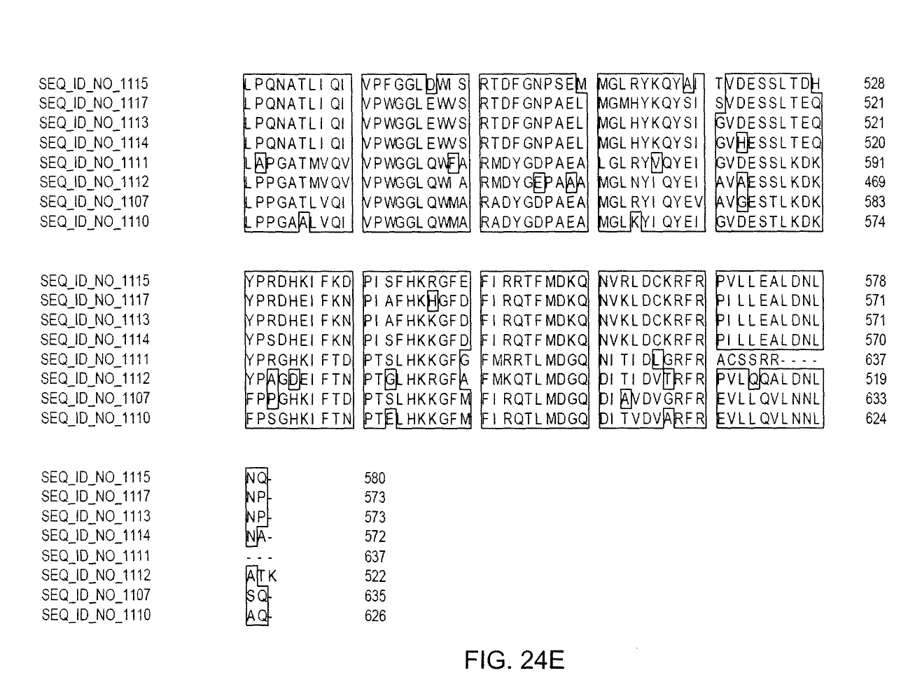

Examples of amino acid sequences of functional homologs of the polypeptide set forth in SEQ ID NO:1107 are provided in FIG. 24 and in the Sequence Listing. Such functional homologs include, for example, GI:56409848 (SEQ ID NO:1108), CeresAnnot:8659084 (SEQ ID NO:1110), GI:31339690 (SEQ ID NO:1111), GI:115434204 (SEQ ID NO:1112), GI:56409850 (SEQ ID NO:1113), GI:162460428 (SEQ ID NO:1114), GI:56409844 (SEQ ID NO:1115), CeresClone:1888614 (SEQ ID NO:1117), CeresAnnot:8740887 (SEQ ID NO:1119), GI:115453815 (SEQ ID NO:1120), GI:125544555 (SEQ ID NO:1121), GI:115445881 (SEQ ID NO:1122), CeresClone:1957829 (SEQ ID NO:1124), CeresAnnot:8674833 (SEQ ID NO:1126), CeresClone:914572 (SEQ ID NO:1128), CeresClone:343865 (SEQ ID NO:1130), CeresClone:322206 (SEQ ID NO:1132), GI:77554062 (SEQ ID NO:1133), or CeresAnnot:8659080 (SEQ ID NO:1135). In some cases, a functional homolog of SEQ ID NO:1107 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:1107.

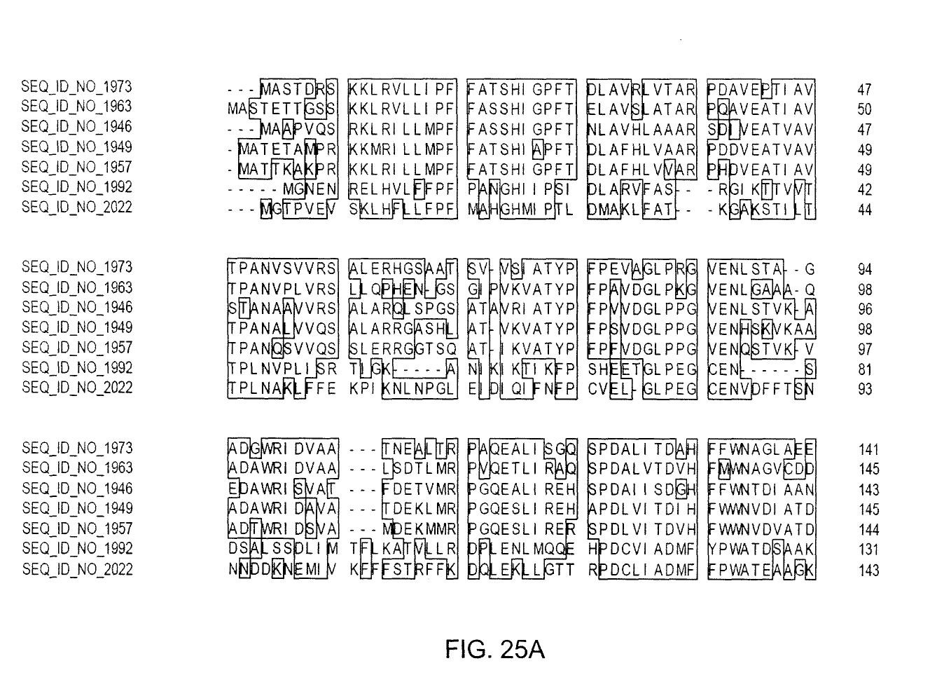

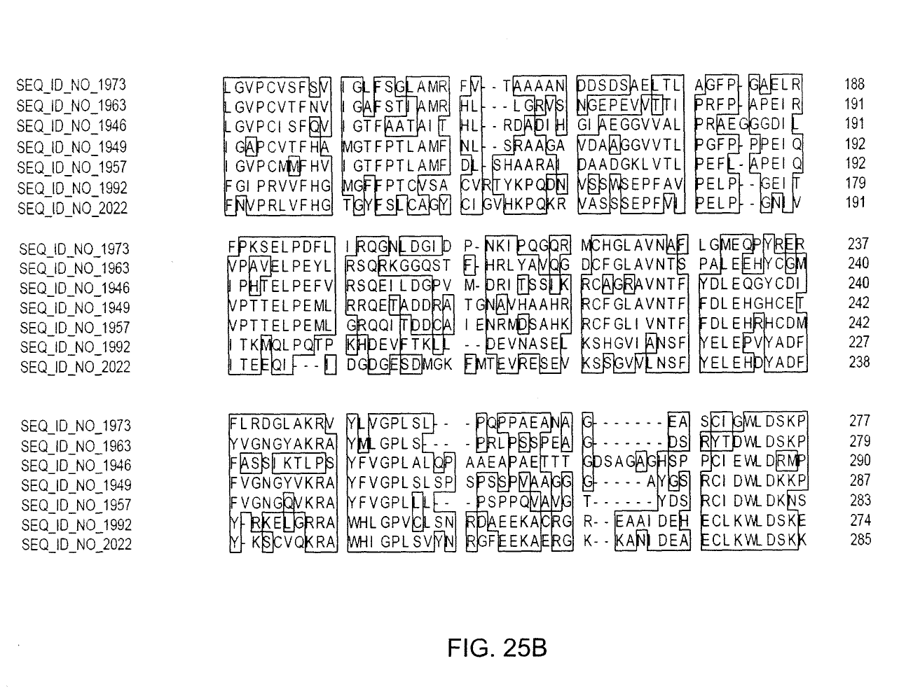

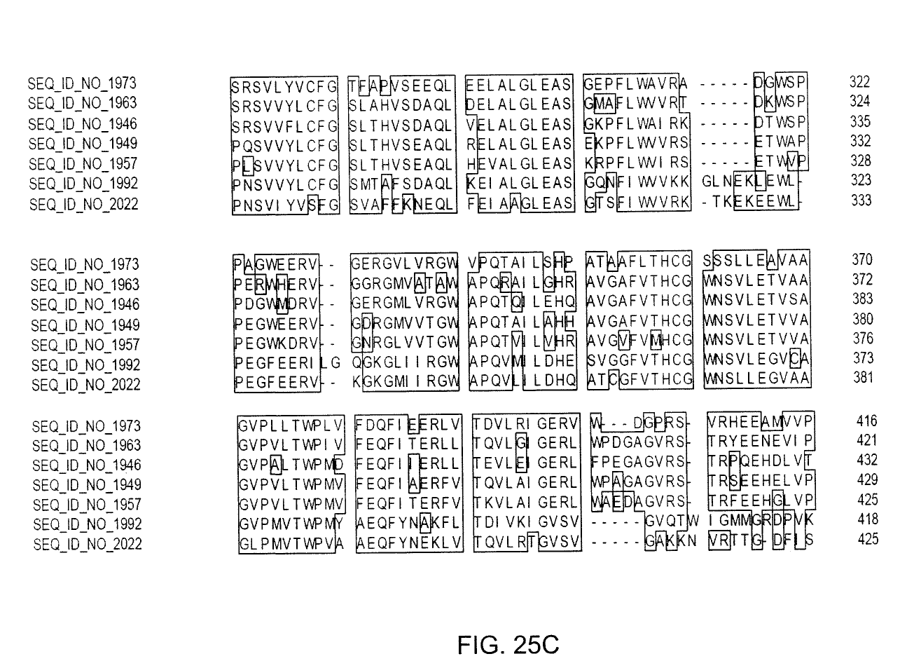

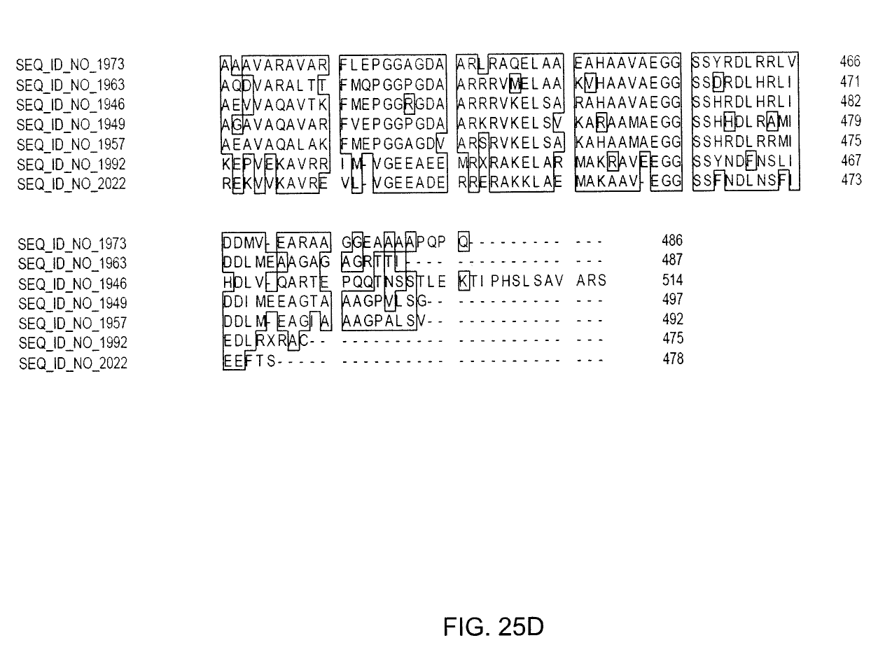

Examples of amino acid sequences of functional homologs of the polypeptide set forth in SEQ ID NO:1946 are provided in FIG. 25 and in the Sequence Listing. Such functional homologs include, for example, CeresClone:1910014 (SEQ ID NO:1948), GI:194697480 (SEQ ID NO:1949), CeresClone:2056605 (SEQ ID NO:1951), CeresClone:1914331 (SEQ ID NO:1953), CeresClone:1886911 (SEQ ID NO:1955), CeresAnnot:8733425 (SEQ ID NO:1957), CeresClone:1754974 (SEQ ID NO:1959), CeresClone:1988346 (SEQ ID NO:1961), CeresClone:918401 (SEQ ID NO:1963), CeresClone:353187 (SEQ ID NO:1965), CeresClone:1726495 (SEQ ID NO:1967), CeresAnnot:8733426 (SEQ ID NO:1969), CeresAnnot:8733429 (SEQ ID NO:1971), GI:194701962 (SEQ ID NO:1972), GI:115464717 (SEQ ID NO:1973), GI:125534461 (SEQ ID NO:1974), GI:115464719 (SEQ ID NO:1975), GI:115464721 (SEQ ID NO:1976), CeresClone:280182 (SEQ ID NO:1978), GI:115456097 (SEQ ID NO:1979), GI:125545696 (SEQ ID NO:1980), CeresAnnot:8632742 (SEQ ID NO:1982), GI:115455283 (SEQ ID NO:1983), CeresAnnot:8667668 (SEQ ID NO:1985), GI:115438785 (SEQ ID NO:1986), GI:125526997 (SEQ ID NO:1987), CeresAnnot:8667665 (SEQ ID NO:1989), GI:157335556 (SEQ ID NO:1990), CeresClone:464566 (SEQ ID NO:1992), CeresClone:678511 (SEQ ID NO:1994), CeresAnnot:1494030 (SEQ ID NO:1996), GI:115438196 (SEQ ID NO:1997), CeresAnnot:8667669 (SEQ ID NO:1999), GI:125571055 (SEQ ID NO:2000), CeresAnnot:1441129 (SEQ ID NO:2002), GI:125526697 (SEQ ID NO:2003), CeresClone:847251 (SEQ ID NO:2005), CeresClone:463175 (SEQ ID NO:2007), GI:125526698 (SEQ ID NO:2008), GI:147839909 (SEQ ID NO:2009), GI:157341038 (SEQ ID NO:2010), GI:50284482 (SEQ ID NO:2011), CeresClone:577260 (SEQ ID NO:2013), CeresAnnot:1461026 (SEQ ID NO:2015), GI:82880420 (SEQ ID NO:2016), GI:18418380 (SEQ ID NO:2017), GI:125524633 (SEQ ID NO:2018), GI:115481308 (SEQ ID NO:2019), CeresAnnot:8656592 (SEQ ID NO:2021), GI:2911049 (SEQ ID NO:2022), GI:125526993 (SEQ ID NO:2023), GI:125531288 (SEQ ID NO:2024), or CeresAnnot:1443816 (SEQ ID NO:2026). In some cases, a functional homolog of SEQ ID NO:1946 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:1946.

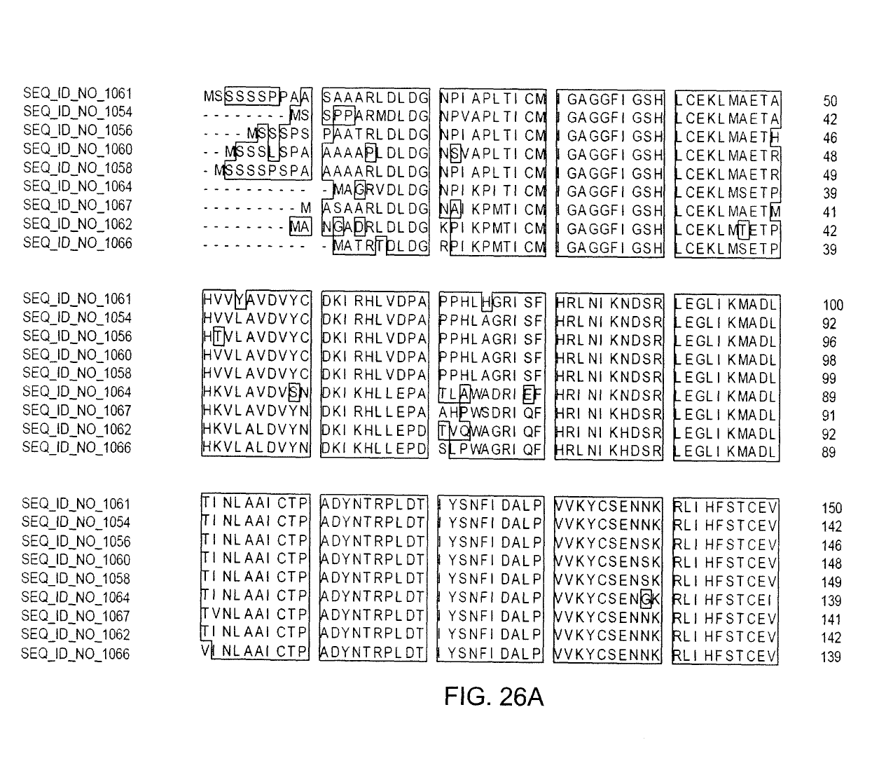

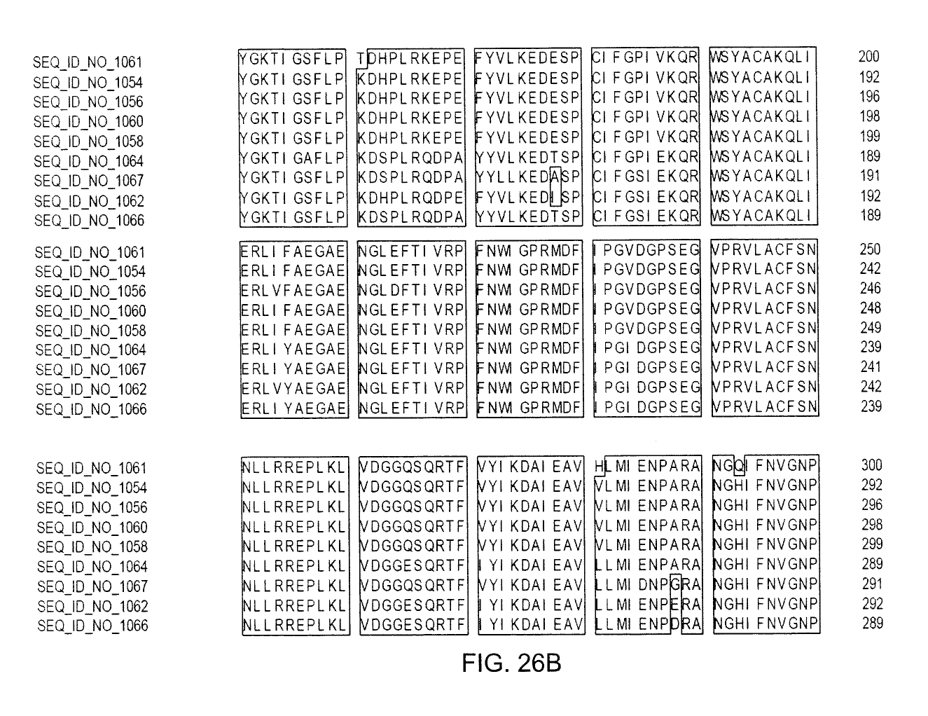

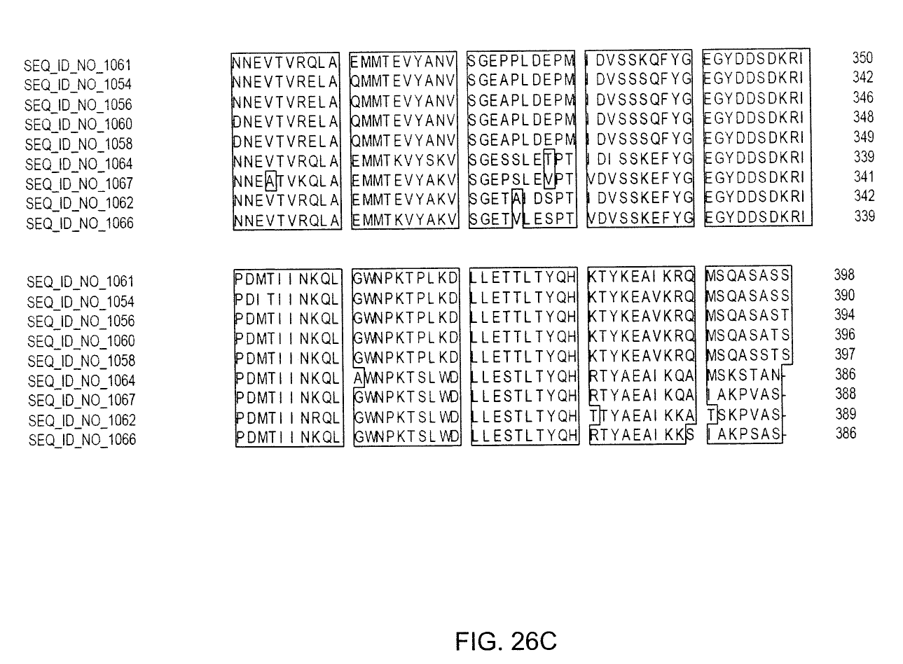

Examples of amino acid sequences of functional homologs of the polypeptide set forth in SEQ ID NO:1054 are provided in FIG. 26 and in the Sequence Listing. Such functional homologs include, for example, CeresClone:300029 (SEQ ID NO:1056), CeresAnnot:8670788 (SEQ ID NO:1058), CeresClone:243057 (SEQ ID NO:1060), GI:115442487 (SEQ ID NO:1061), GI:21594350 (SEQ ID NO:1062), CeresAnnot:8460479 (SEQ ID NO:1064), CeresClone:1948444 (SEQ ID NO:1066), GI:225465764 (SEQ ID NO:1067), CeresClone:1995643 (SEQ ID NO:1069), GI:162462229 (SEQ ID NO:1070), GI:125529268 (SEQ ID NO:1071), GI:194689614 (SEQ ID NO:1072), CeresAnnot:863641 (SEQ ID NO:1074), CeresClone:1969770 (SEQ ID NO:1076), GI:14596185 (SEQ ID NO:1077), GI:225436371 (SEQ ID NO:1078), GI:85718018 (SEQ ID NO:1079), CeresClone:28205 (SEQ ID NO:1081), CeresAnnot:1541782 (SEQ ID NO:1083), CeresClone:1936952 (SEQ ID NO:1085), CeresClone:1459647 (SEQ ID NO:1087), GI:37379419 (SEQ ID NO:1088), CeresAnnot:1460446 (SEQ ID NO:1090), CeresAnnot:1488364 (SEQ ID NO:1092), GI:45935133 (SEQ ID NO:1093), CeresClone:1874074 (SEQ ID NO:1095), GI:225464984 (SEQ ID NO:1096), GI:147771536 (SEQ ID NO:1097), CeresClone:6892 (SEQ ID NO:1099), CeresClone:1047104 (SEQ ID NO:1101), GI:168063797 (SEQ ID NO:1102), CeresClone:100063067 (SEQ ID NO:1104), or GI:85376636 (SEQ ID NO:1105). In some cases, a functional homolog of SEQ ID NO:1054 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:1054.

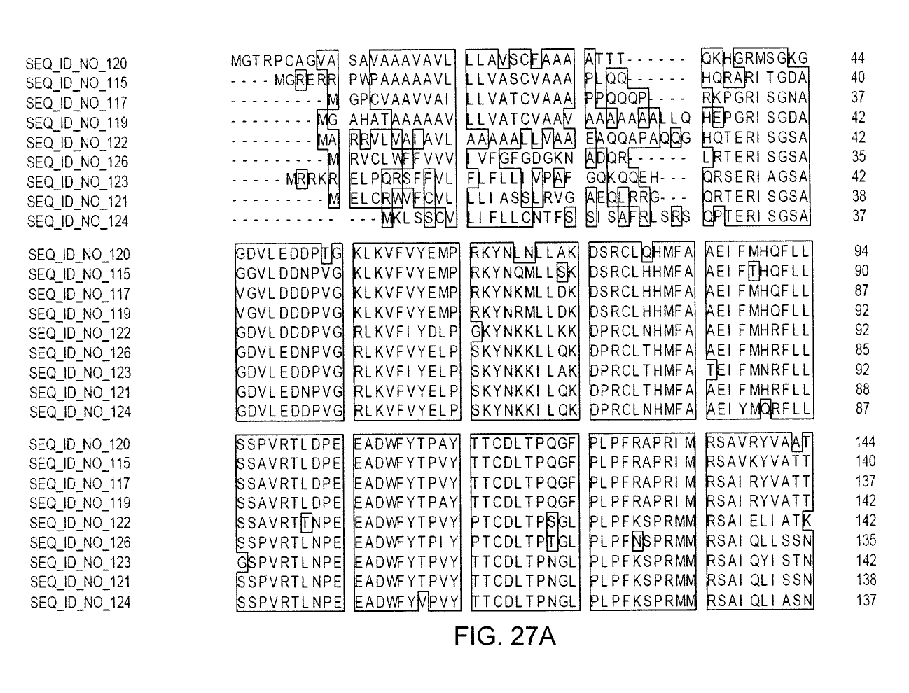

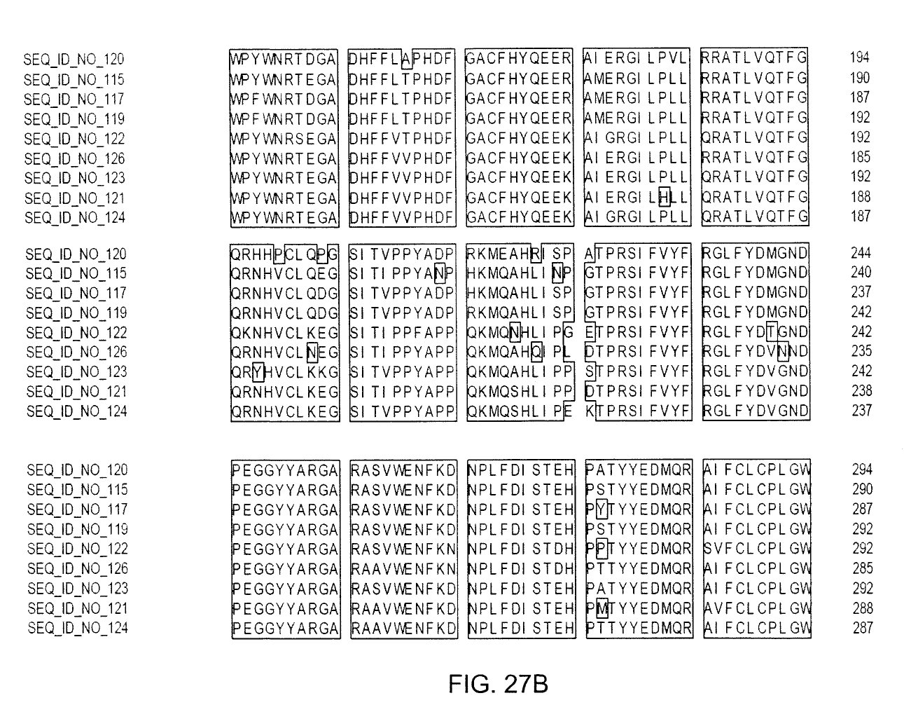

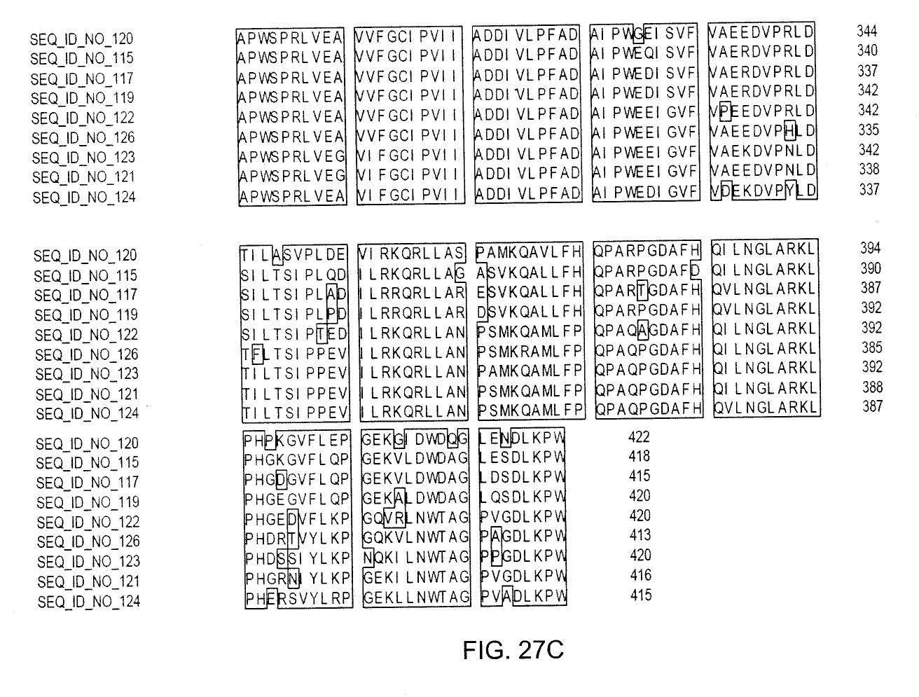

Examples of amino acid sequences of functional homologs of the polypeptide set forth in SEQ ID NO:115 are provided in FIG. 27 and in the Sequence Listing. Such functional homologs include, for example, CeresAnnot:8670354 (SEQ ID NO:117), CeresClone:363483 (SEQ ID NO:119), GI:115441965 (SEQ ID NO:120), GI:225456511 (SEQ ID NO:121), GI:150246869 (SEQ ID NO:122), GI:116793780 (SEQ ID NO:123), GI:18424516 (SEQ ID NO:124), CeresAnnot:1447833 (SEQ ID NO:126), CeresClone:1904977 (SEQ ID NO:128), GI:125528940 (SEQ ID NO:129), CeresAnnot:8670355 (SEQ ID NO:131), CeresClone:313262 (SEQ ID NO:133), CeresClone:1807519 (SEQ ID NO:135), CeresAnnot:8670356 (SEQ ID NO:137), CeresAnnot:8733886 (SEQ ID NO:139), GI:212276027 (SEQ ID NO:140), GI:115458146 (SEQ ID NO:141), GI:194708308 (SEQ ID NO:142), CeresClone:1471197 (SEQ ID NO:144), CeresClone:244622 (SEQ ID NO:146), GI:194700342 (SEQ ID NO:147), GI:194704652 (SEQ ID NO:148), GI:115441969 (SEQ ID NO:149), GI:49389081 (SEQ ID NO:150), CeresClone:1788372 (SEQ ID NO:152), CeresAnnot:8679199 (SEQ ID NO:154), GI:125582313 (SEQ ID NO:155), CeresAnnot:8699946 (SEQ ID NO:157), GI:212275360 (SEQ ID NO:158), CeresClone:246342 (SEQ ID NO:160), CeresClone:1875736 (SEQ ID NO:162), GI:115441967 (SEQ ID NO:163), CeresAnnot:8733887 (SEQ ID NO:165), CeresClone:2008277 (SEQ ID NO:167), CeresAnnot:1522137 (SEQ ID NO:169), CeresClone:280575 (SEQ ID NO:171), CeresAnnot:1522133 (SEQ ID NO:173), CeresAnnot:8460007 (SEQ ID NO:175), GI:194706890 (SEQ ID NO:176), CeresClone:34861 (SEQ ID NO:178), CeresClone:206413 (SEQ ID NO:180), CeresAnnot:1491074 (SEQ ID NO:182), GI:60657598 (SEQ ID NO:183), CeresAnnot:8733056 (SEQ ID NO:185), CeresAnnot:8458833 (SEQ ID NO:187), CeresClone:1818144 (SEQ ID NO:189), CeresAnnot:1468764 (SEQ ID NO:191), CeresAnnot:1468760 (SEQ ID NO:193), GI:115481310 (SEQ ID NO:194), GI:21671946 (SEQ ID NO:195), GI:18642697 (SEQ ID NO:196), GI:168000965 (SEQ ID NO:197), GI:23821292 (SEQ ID NO:198), GI:157359959 (SEQ ID NO:199), GI:125539672 (SEQ ID NO:200), GI:10176877 (SEQ ID NO:201), CeresClone:2013135 (SEQ ID NO:203), CeresClone:910275 (SEQ ID NO:205), CeresAnnot:1438255 (SEQ ID NO:207), GI:39725600 (SEQ ID NO:208), or GI:110288702 (SEQ ID NO:209). In some cases, a functional homolog of SEQ ID NO:115 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:115.

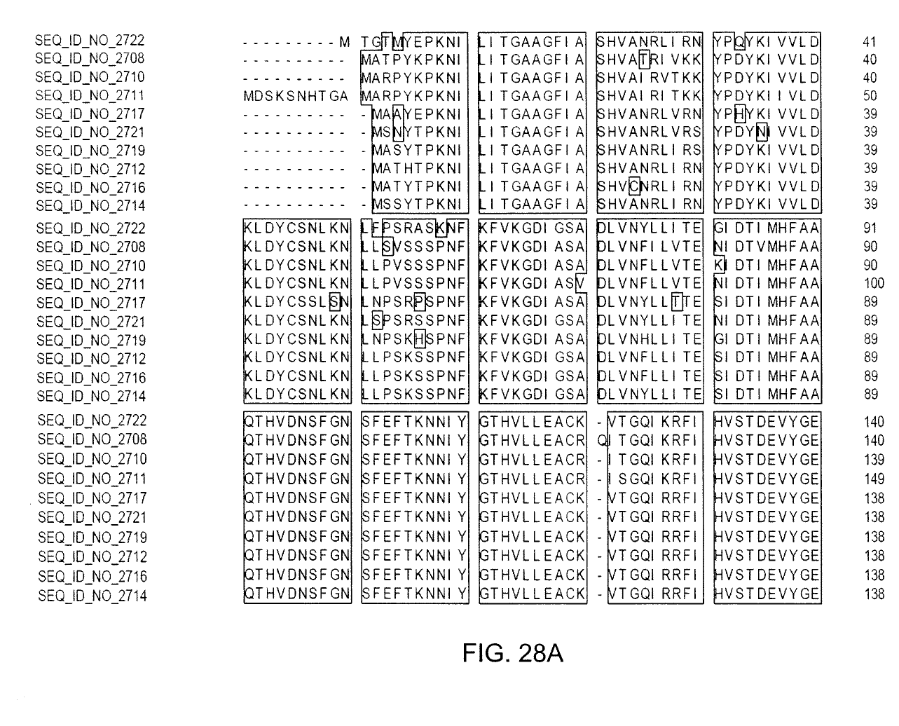

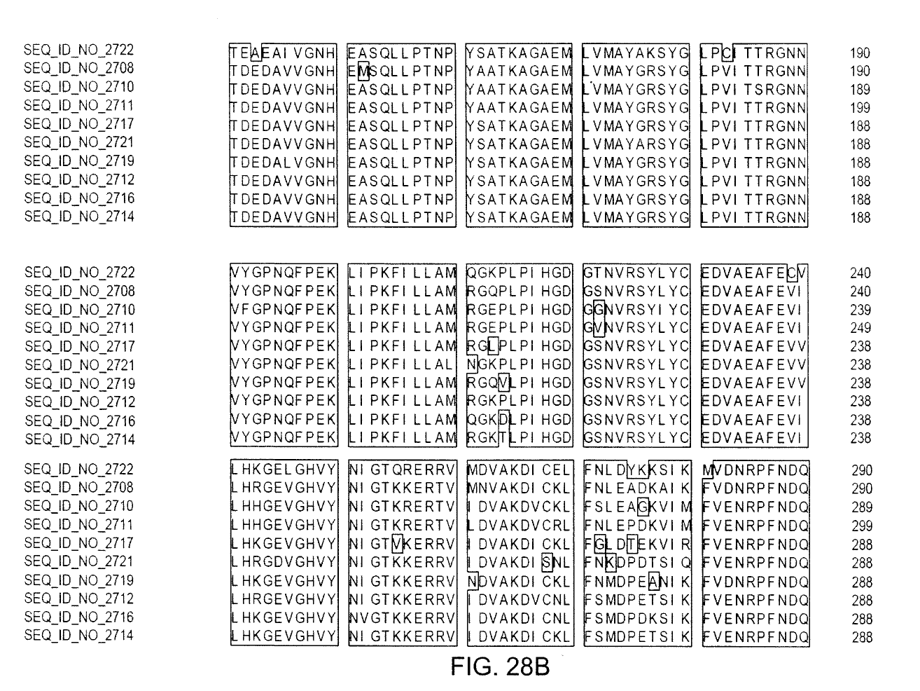

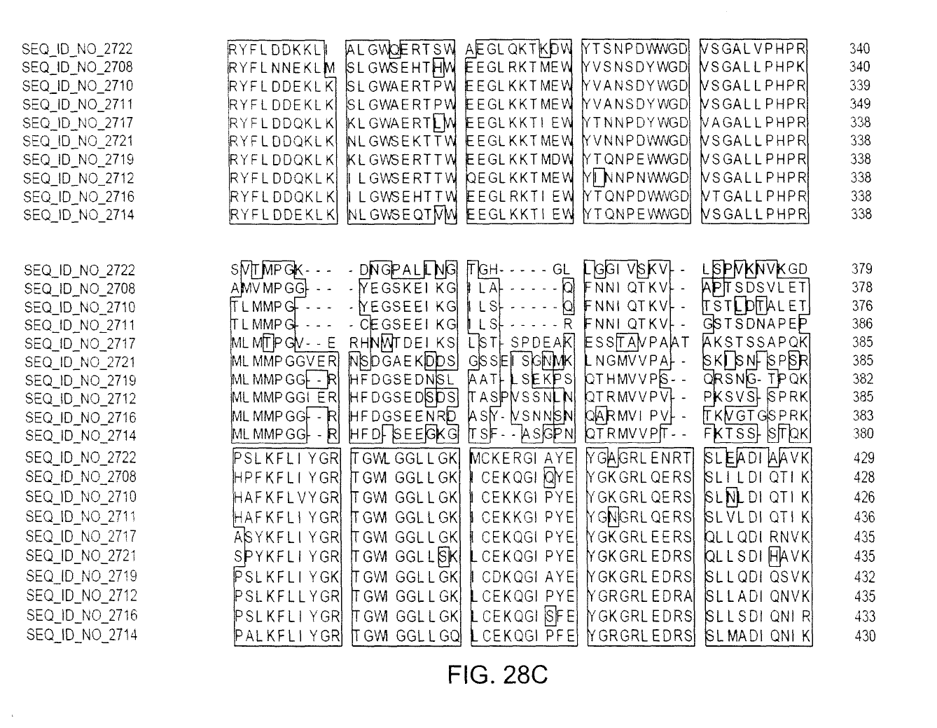

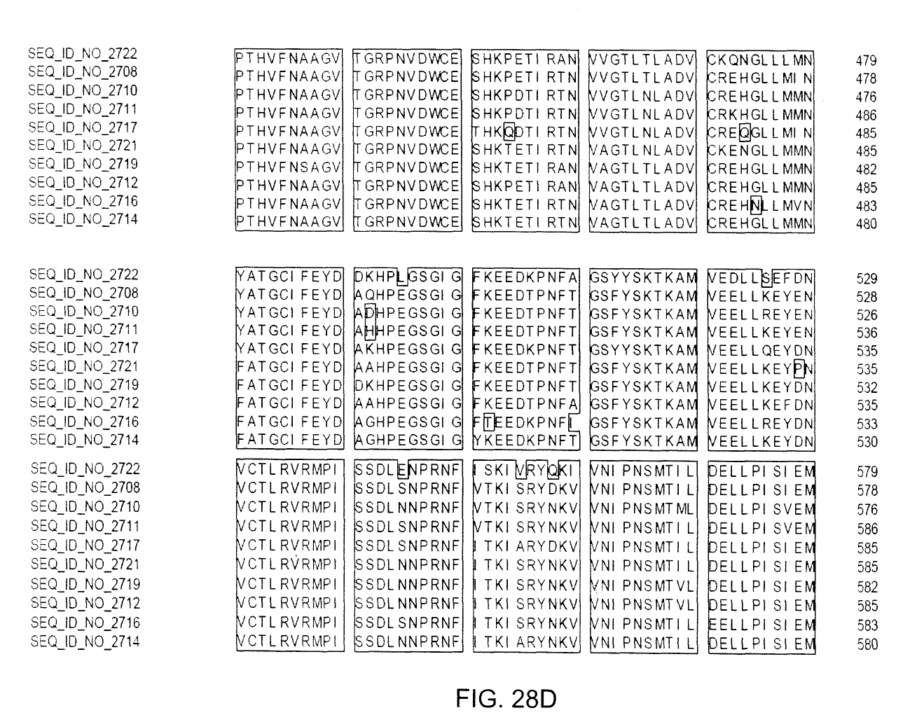

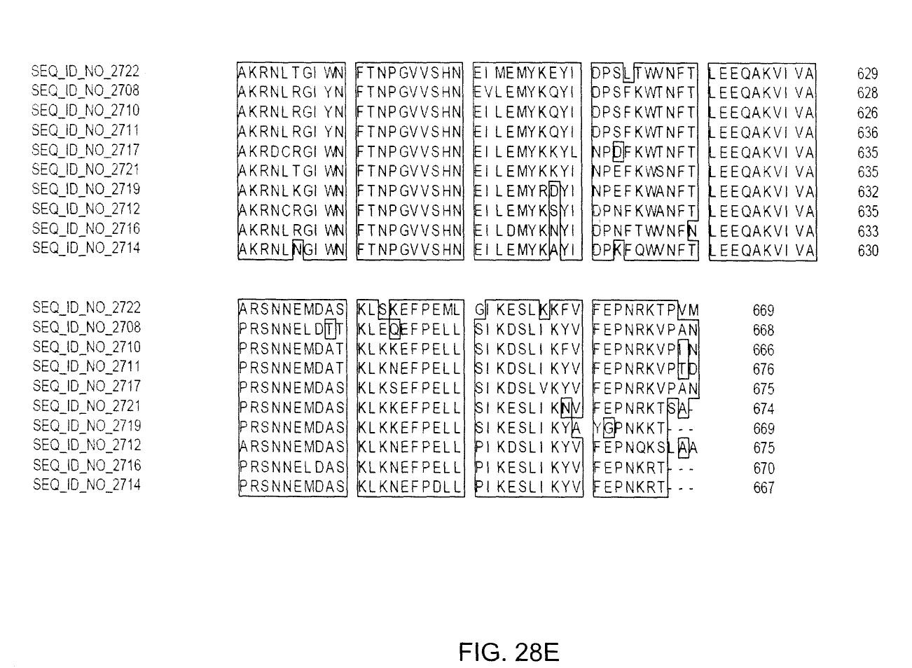

Examples of amino acid sequences of functional homologs of the polypeptide set forth in SEQ ID NO:2708 are provided in FIG. 28 and in the Sequence Listing. Such functional homologs include, for example, CeresAnnot:8726940 (SEQ ID NO:2710), GI:212274887 (SEQ ID NO:2711), GI:225461808 (SEQ ID NO:2712), CeresClone:1920439 (SEQ ID NO:2714), CeresAnnot:1482328 (SEQ ID NO:2716), GI:115452247 (SEQ ID NO:2717), CeresAnnot:869962 (SEQ ID NO:2719), CeresAnnot:8461116 (SEQ ID NO:2721), GI:168046471 (SEQ ID NO:2722), CeresClone:1792946 (SEQ ID NO:2724), CeresClone:351260 (SEQ ID NO:2726), CeresAnnot:8641839 (SEQ ID NO:2728), CeresClone:1951869 (SEQ ID NO:2730), CeresAnnot:8641817 (SEQ ID NO:2732), CeresClone:1920196 (SEQ ID NO:2734), GI:15231926 (SEQ ID NO:2735), GI:42562732 (SEQ ID NO:2736), CeresClone:1924612 (SEQ ID NO:2738), GI:168054569 (SEQ ID NO:2739), GI:168028758 (SEQ ID NO:2740), GI:225464343 (SEQ ID NO:2741), CeresAnnot:1480867 (SEQ ID NO:2743), CeresAnnot:8464234 (SEQ ID NO:2745), or GI:116056148 (SEQ ID NO:2746). In some cases, a functional homolog of SEQ ID NO:2708 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2708.

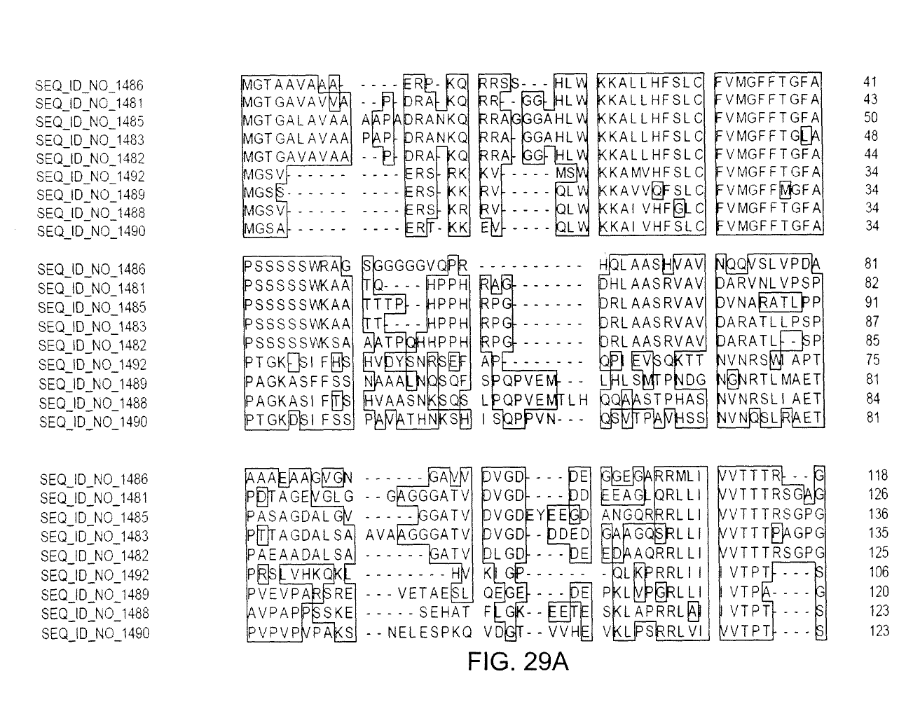

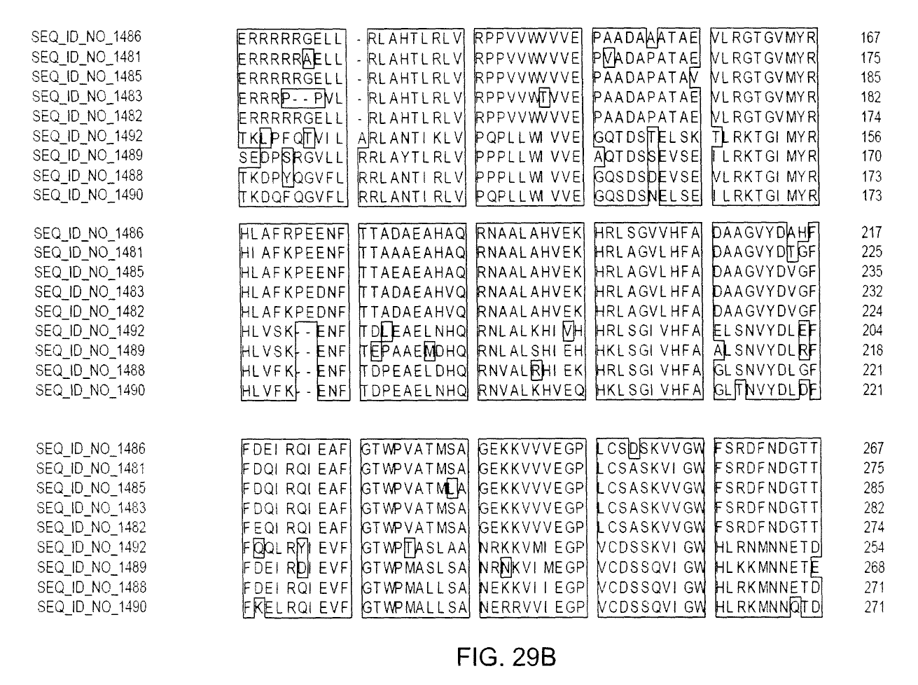

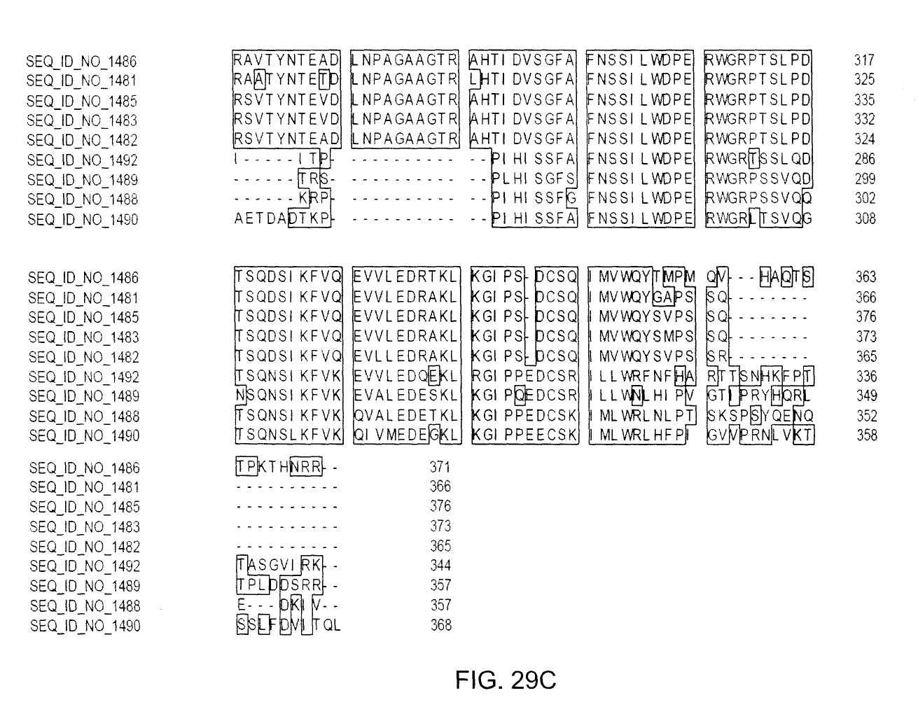

Examples of amino acid sequences of functional homologs of the polypeptide set forth in SEQ ID NO:1481 are provided in FIG. 29 and in the Sequence Listing. Such functional homologs include, for example, GI:194701352 (SEQ ID NO:1482), GI:63087724 (SEQ ID NO:1483), CeresAnnot:8724648 (SEQ ID NO:1485), GI:115461821 (SEQ ID NO:1486), CeresAnnot:1528801 (SEQ ID NO:1488), GI:225439900 (SEQ ID NO:1489), GI:33771692 (SEQ ID NO:1490), CeresClone:547127 (SEQ ID NO:1492), GI:162459701 (SEQ ID NO:1493), CeresAnnot:8641721 (SEQ ID NO:1495), CeresClone:1788804 (SEQ ID NO:1497), GI:115452359 (SEQ ID NO:1498), CeresClone:1819717 (SEQ ID NO:1500), CeresClone:1558521 (SEQ ID NO:1502), GI:108707574 (SEQ ID NO:1503), CeresClone:575502 (SEQ ID NO:1505), GI:150246871 (SEQ ID NO:1506), GI:60657596 (SEQ ID NO:1507), GI:145408198 (SEQ ID NO:1508), GI:63087726 (SEQ ID NO:1509), CeresAnnot:1538289 (SEQ ID NO:1511), GI:60657594 (SEQ ID NO:1512), CeresAnnot:1475351 (SEQ ID NO:1514), GI:61673388 (SEQ ID NO:1515), or GI:15228084 (SEQ ID NO:1516). In some cases, a functional homolog of SEQ ID NO:1481 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:1481.

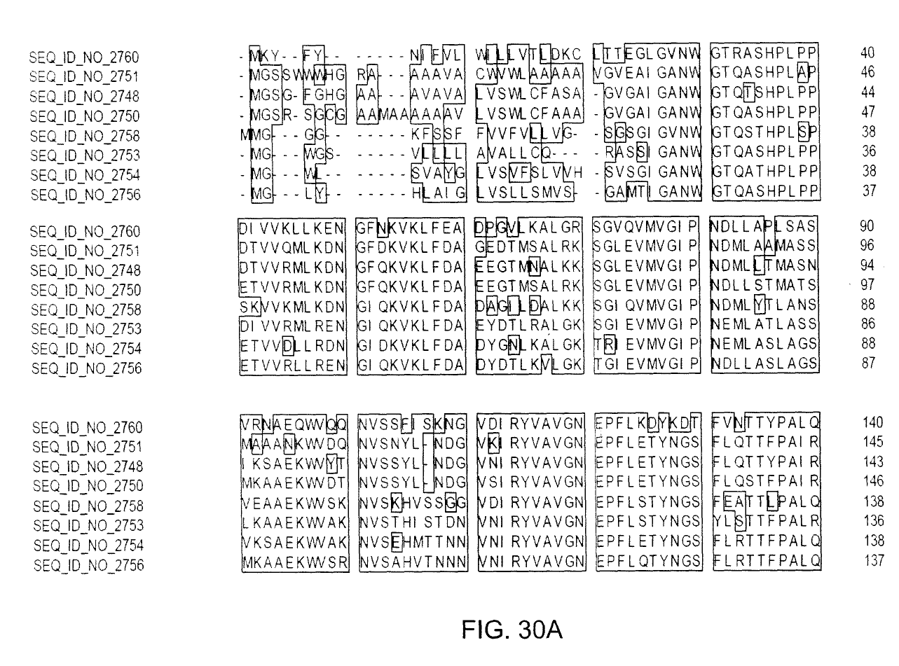

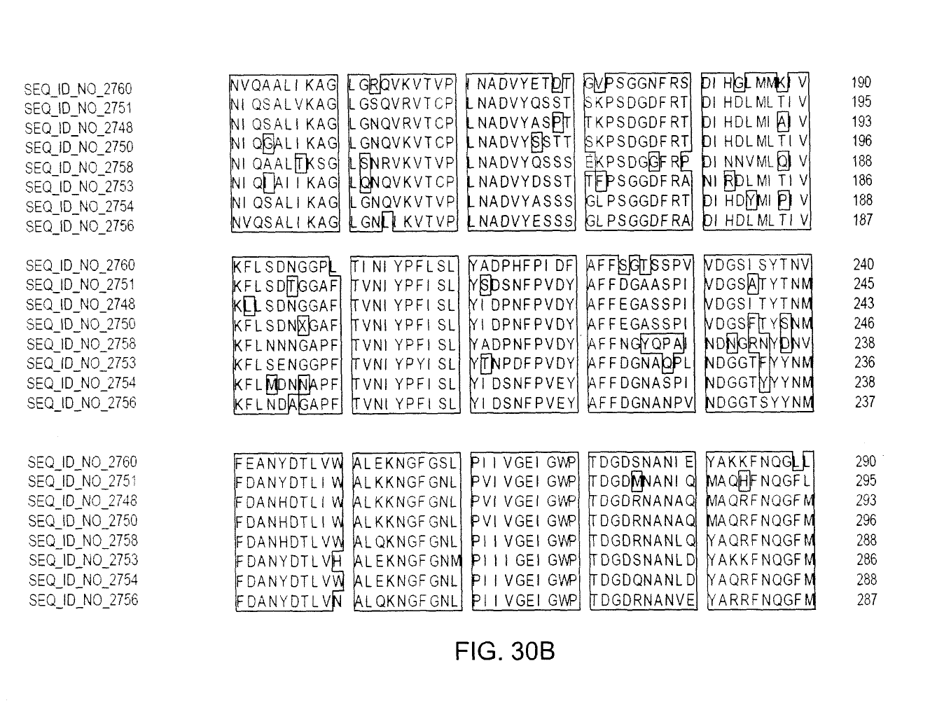

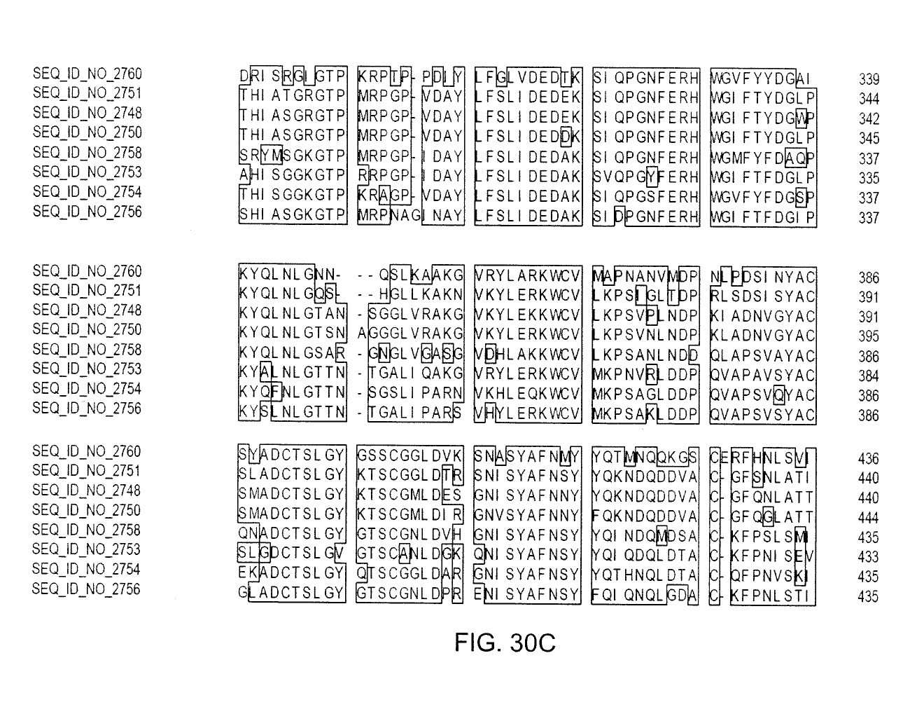

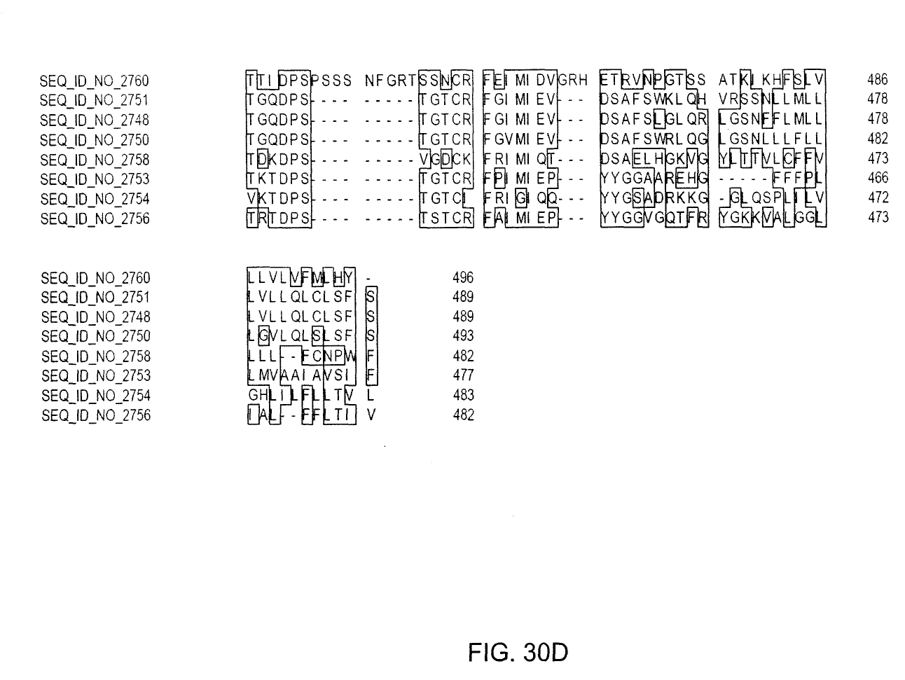

Examples of amino acid sequences of functional homologs of the polypeptide set forth in SEQ ID NO:2748 are provided in FIG. 30 and in the Sequence Listing. Such functional homologs include, for example, CeresClone:1790340 (SEQ ID NO:2748), CeresClone:327425 (SEQ ID NO:2750), GI:115444129 (SEQ ID NO:2751), CeresClone:1152436 (SEQ ID NO:2753), GI:157353536 (SEQ ID NO:2754), CeresAnnot:1482019 (SEQ ID NO:2756), CeresClone:651435 (SEQ ID NO:2758), CeresAnnot:8458215 (SEQ ID NO:2760), CeresClone:114877 (SEQ ID NO:2762), CeresClone:2007431 (SEQ ID NO:2764), CeresClone:1875367 (SEQ ID NO:2766), CeresClone:1882421 (SEQ ID NO:2768), CeresAnnot:1469752 (SEQ ID NO:2770), GI:115468738 (SEQ ID NO:2771), GI:46518473 (SEQ ID NO:2772), GI:15224778 (SEQ ID NO:2773), CeresClone:1810424 (SEQ ID NO:2775), CeresClone:1559140 (SEQ ID NO:2777), GI:16604491 (SEQ ID NO:2778), CeresClone:1795733 (SEQ ID NO:2780), GI:115475361 (SEQ ID NO:2781), GI:125560593 (SEQ ID NO:2782), CeresAnnot:1507103 (SEQ ID NO:2784), CeresAnnot:1453959 (SEQ ID NO:2786), CeresAnnot:1480967 (SEQ ID NO:2788), CeresAnnot:1476972 (SEQ ID NO:2790), GI:15235840 (SEQ ID NO:2791), GI:157342450 (SEQ ID NO:2792), CeresAnnot:1455089 (SEQ ID NO:2794), CeresClone:1581235 (SEQ ID NO:2796), GI:116789946 (SEQ ID NO:2797), CeresClone:790682 (SEQ ID NO:2799), CeresClone:1926644 (SEQ ID NO:2801), GI:125544205 (SEQ ID NO:2802), GI:30017525 (SEQ ID NO:2803), GI:225441645 (SEQ ID NO:2804), CeresClone:616565 (SEQ ID NO:2806), GI:225438519 (SEQ ID NO:2807), CeresAnnot:1523133 (SEQ ID NO:2809), GI:15242078 (SEQ ID NO:2810), GI:115454419 (SEQ ID NO:2811), CeresAnnot:1532670 (SEQ ID NO:2813), CeresClone:1788618 (SEQ ID NO:2815), GI:225445328 (SEQ ID NO:2816), CeresClone:1596827 (SEQ ID NO:2818), GI:124365582 (SEQ ID NO:2819), CeresClone:1786326 (SEQ ID NO:2821), GI:48374979 (SEQ ID NO:2822), GI:18483232 (SEQ ID NO:2823), CeresClone:1597530 (SEQ ID NO:2825), GI:37223500 (SEQ ID NO:2826), CeresAnnot:1479207 (SEQ ID NO:2828), CeresAnnot:8705326 (SEQ ID NO:2830), CeresAnnot:8458673 (SEQ ID NO:2832), GI:115470771 (SEQ ID NO:2833), CeresAnnot:1477929 (SEQ ID NO:2835), GI:194691368 (SEQ ID NO:2836), CeresAnnot:1456572 (SEQ ID NO:2838), CeresClone:940271 (SEQ ID NO:2840), CeresClone:1790079 (SEQ ID NO:2842), CeresAnnot:1509765 (SEQ ID NO:2844), GI:77551687 (SEQ ID NO:2845), GI:125577601 (SEQ ID NO:2846), GI:125534858 (SEQ ID NO:2847), CeresClone:1551402 (SEQ ID NO:2849), GI:42563478 (SEQ ID NO:2850), GI:125597725 (SEQ ID NO:2851), GI:15217739 (SEQ ID NO:2852), GI:15230097 (SEQ ID NO:2853), CeresClone:445269 (SEQ ID NO:2855), GI:15238768 (SEQ ID NO:2856), GI:115465107 (SEQ ID NO:2857), GI:5042412 (SEQ ID NO:2858), GI:194705838 (SEQ ID NO:2859), CeresAnnot:1533786 (SEQ ID NO:2861), GI:124360899 (SEQ ID NO:2862), GI:115475910 (SEQ ID NO:2863), GI:125561098 (SEQ ID NO:2864), CeresAnnot:887555 (SEQ ID NO:2866), GI:115478288 (SEQ ID NO:2867), GI:147845562 (SEQ ID NO:2868), GI:157329499 (SEQ ID NO:2869), GI:168046763 (SEQ ID NO:2870), CeresAnnot:1480316 (SEQ ID NO:2872), GI:168003834 (SEQ ID NO:2873), CeresAnnot:877895 (SEQ ID NO:2875), GI:125602994 (SEQ ID NO:2876), GI:89257617 (SEQ ID NO:2877), or GI:168041780 (SEQ ID NO:2878). In some cases, a functional homolog of SEQ ID NO:2748 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2748.

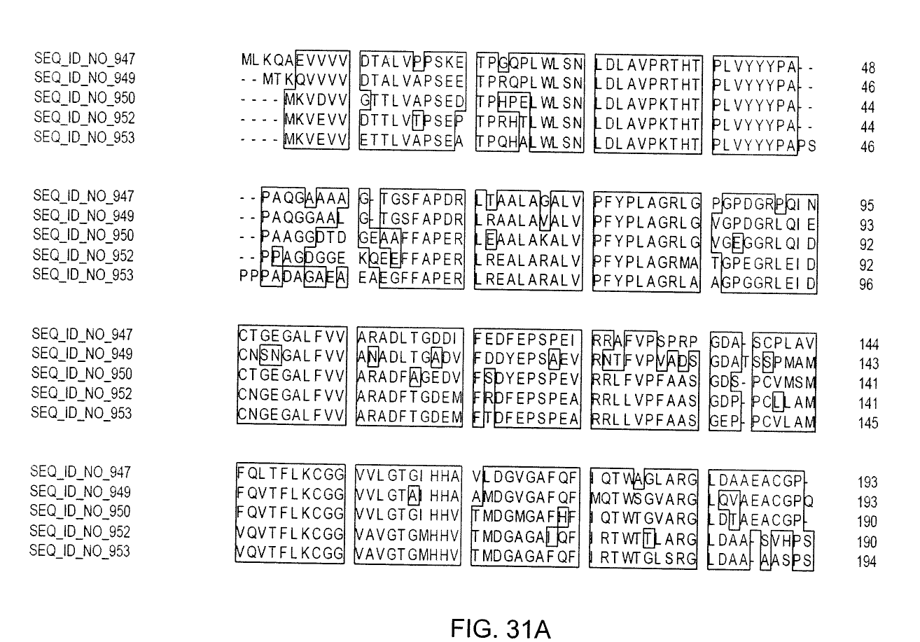

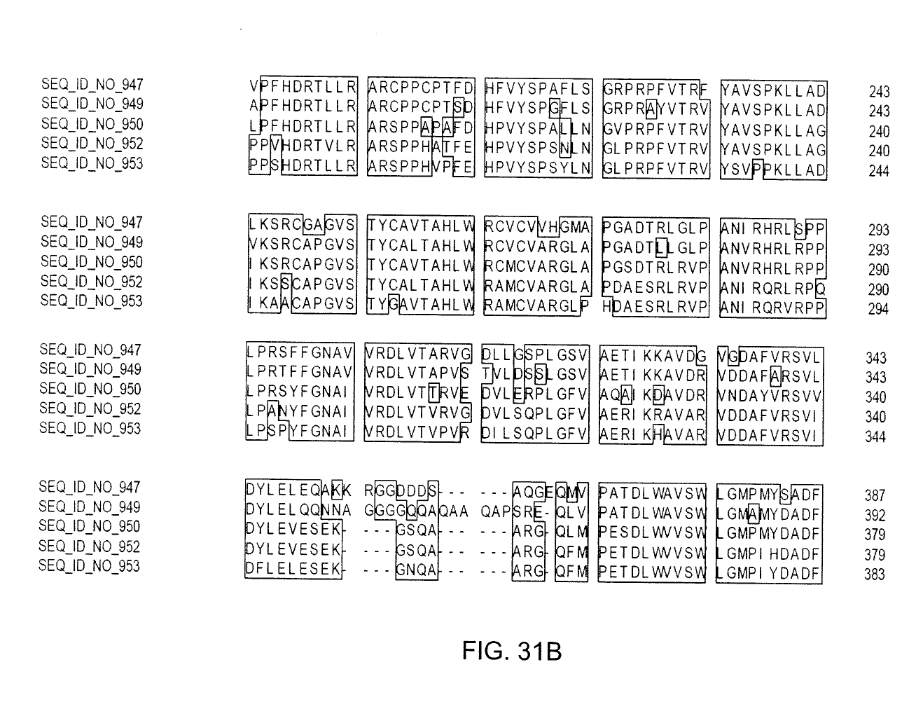

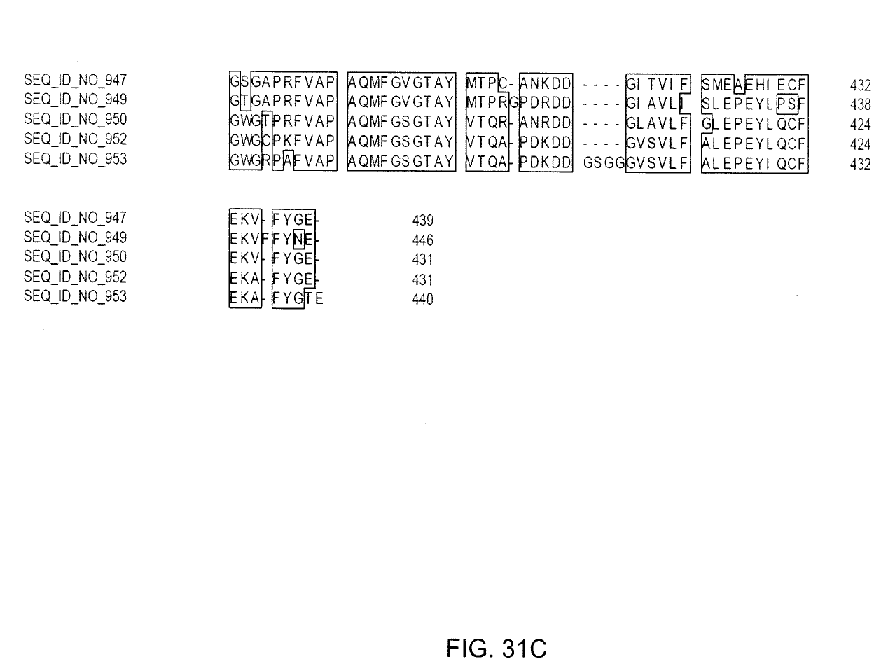

Examples of amino acid sequences of functional homologs of the polypeptide set forth in SEQ ID NO:947 are provided in FIG. 31 and in the Sequence Listing. Such functional homologs include, for example, CeresAnnot:8713285 (SEQ ID NO:949), GI:194706702 (SEQ ID NO:950), CeresClone:569098 (SEQ ID NO:952), GI:115479225 (SEQ ID NO:953), CeresClone:298496 (SEQ ID NO:955), CeresClone:1534877 (SEQ ID NO:957), CeresAnnot:8713281 (SEQ ID NO:959), GI:125563758 (SEQ ID NO:960), CeresClone:333204 (SEQ ID NO:962), CeresAnnot:8654155 (SEQ ID NO:964), GI:125554333 (SEQ ID NO:965), CeresAnnot:8658834 (SEQ ID NO:967), GI:115466798 (SEQ ID NO:968), CeresClone:1788581 (SEQ ID NO:970), CeresClone:245537 (SEQ ID NO:972), CeresAnnot:8682006 (SEQ ID NO:974), CeresClone:1032064 (SEQ ID NO:976), CeresClone:725011 (SEQ ID NO:978), CeresAnnot:8654153 (SEQ ID NO:980), GI:115477609 (SEQ ID NO:981), CeresClone:1903575 (SEQ ID NO:983), CeresClone:221247 (SEQ ID NO:985), GI:194701434 (SEQ ID NO:986), GI:125562404 (SEQ ID NO:987), CeresAnnot:8754033 (SEQ ID NO:989), CeresAnnot:8658583 (SEQ ID NO:991), CeresAnnot:8658570 (SEQ ID NO:993), CeresAnnot:8658589 (SEQ ID NO:995), CeresAnnot:8658592 (SEQ ID NO:997), GI:125554336 (SEQ ID NO:998), CeresAnnot:8658566 (SEQ ID NO:1000), CeresAnnot:8653415 (SEQ ID NO:1002), GI:125554335 (SEQ ID NO:1003), CeresAnnot:8735477 (SEQ ID NO:1005), CeresClone:1995174 (SEQ ID NO:1007), GI:194707128 (SEQ ID NO:1008), GI:115466808 (SEQ ID NO:1009), CeresAnnot:8701741 (SEQ ID NO:1011), CeresClone:788394 (SEQ ID NO:1013), GI:115459250 (SEQ ID NO:1014), CeresClone:764333 (SEQ ID NO:1016), CeresAnnot:8735476 (SEQ ID NO:1018), CeresAnnot:8735473 (SEQ ID NO:1020), CeresClone:465877 (SEQ ID NO:1022), GI:194700936 (SEQ ID NO:1023), GI:115484487 (SEQ ID NO:1024), GI:125576435 (SEQ ID NO:1025), GI:115447257 (SEQ ID NO:1026), GI:82568711 (SEQ ID NO:1027), CeresClone:786100 (SEQ ID NO:1029), CeresClone:1920450 (SEQ ID NO:1031), CeresClone:1920870 (SEQ ID NO:1033), GI:32400295 (SEQ ID NO:1034), CeresClone:1920088 (SEQ ID NO:1036), CeresClone:741711 (SEQ ID NO:1038), GI:32400293 (SEQ ID NO:1039), CeresClone:886187 (SEQ ID NO:1041), GI:115475267 (SEQ ID NO:1042), GI:6469032 (SEQ ID NO:1043), GI:73671233 (SEQ ID NO:1044), GI:70663907 (SEQ ID NO:1045), GI:212722240 (SEQ ID NO:1046), CeresClone:1691303 (SEQ ID NO:1048), GI:32400291 (SEQ ID NO:1049), GI:40644899 (SEQ ID NO:1050), GI:40253398 (SEQ ID NO:1051), or GI:139538848 (SEQ ID NO:1052). In some cases, a functional homolog of SEQ ID NO:947 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:947.

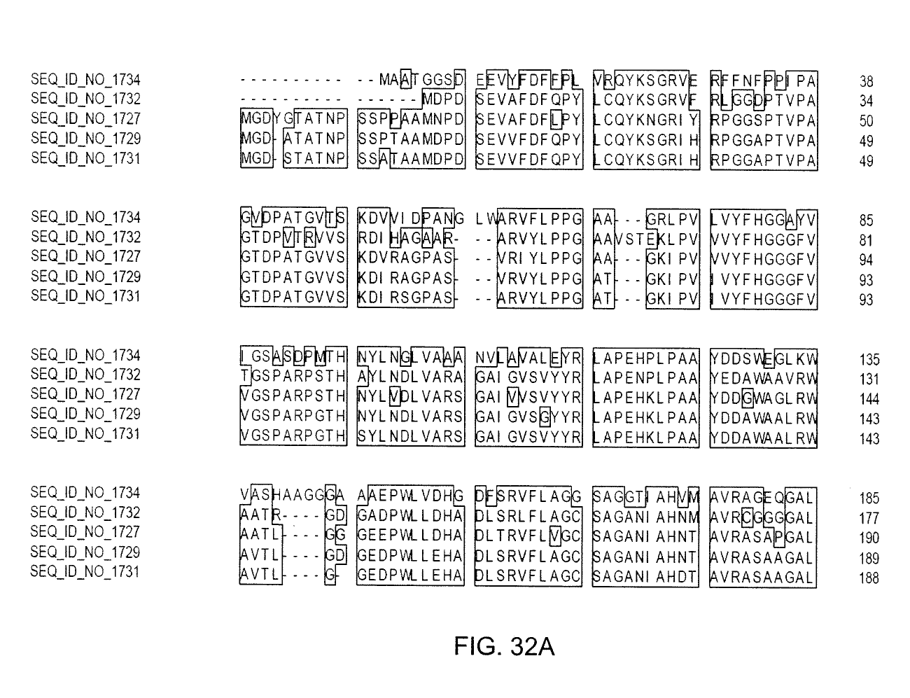

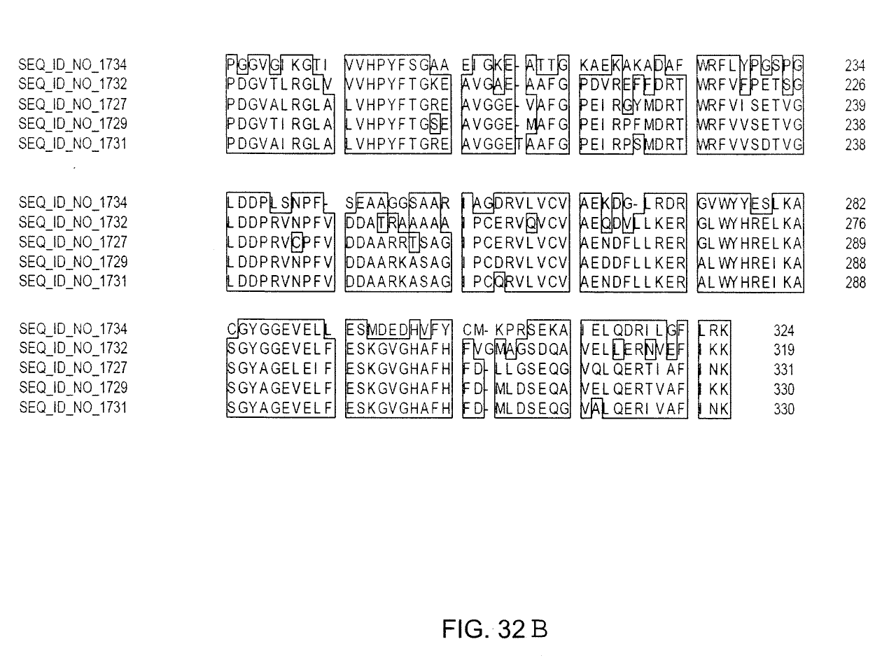

Examples of amino acid sequences of functional homologs of the polypeptide set forth in SEQ ID NO:1727 are provided in FIG. 32 and in the Sequence Listing. Such functional homologs include, for example, CeresAnnot:8735802 (SEQ ID NO:1729), CeresClone:292537 (SEQ ID NO:1731), GI:51090388 (SEQ ID NO:1732), CeresClone:732524 (SEQ ID NO:1734), GI:115467066 (SEQ ID NO:1735), CeresAnnot:8735801 (SEQ ID NO:1737), CeresClone:1807095 (SEQ ID NO:1739), CeresClone:2024952 (SEQ ID NO:1741), CeresClone:281110 (SEQ ID NO:1743), CeresAnnot:8735800 (SEQ ID NO:1745), GI:115479613 (SEQ ID NO:1746), GI:82697943 (SEQ ID NO:1747), CeresAnnot:8713623 (SEQ ID NO:1749), CeresClone:243558 (SEQ ID NO:1751), CeresClone:816193 (SEQ ID NO:1753), CeresAnnot:8654590 (SEQ ID NO:1755), CeresClone:1725434 (SEQ ID NO:1757), GI:125596482 (SEQ ID NO:1758), GI:115476880 (SEQ ID NO:1759), CeresClone:1814324 (SEQ ID NO:1761), GI:125554537 (SEQ ID NO:1762), CeresClone:1031198 (SEQ ID NO:1764), GI:115479609 (SEQ ID NO:1765), GI:116310075 (SEQ ID NO:1766), GI:115476872 (SEQ ID NO:1767), CeresClone:1989171 (SEQ ID NO:1769), GI:115479603 (SEQ ID NO:1770), CeresClone:100896047 (SEQ ID NO:1772), GI:42408206 (SEQ ID NO:1773), GI:125564014 (SEQ ID NO:1774), CeresClone:457826 (SEQ ID NO:1776), GI:157335490 (SEQ ID NO:1777), CeresClone:1017543 (SEQ ID NO:1779), CeresClone:219954 (SEQ ID NO:1781), CeresAnnot:8654584 (SEQ ID NO:1783), GI:115476870 (SEQ ID NO:1784), CeresClone:1446903 (SEQ ID NO:1786), CeresClone:1907709 (SEQ ID NO:1788), CeresAnnot:8722921 (SEQ ID NO:1790), CeresAnnot:8735798 (SEQ ID NO:1792), CeresAnnot:8713597 (SEQ ID NO:1794), or GI:225428761 (SEQ ID NO:1795). In some cases, a functional homolog of SEQ ID NO:1727 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:1727.

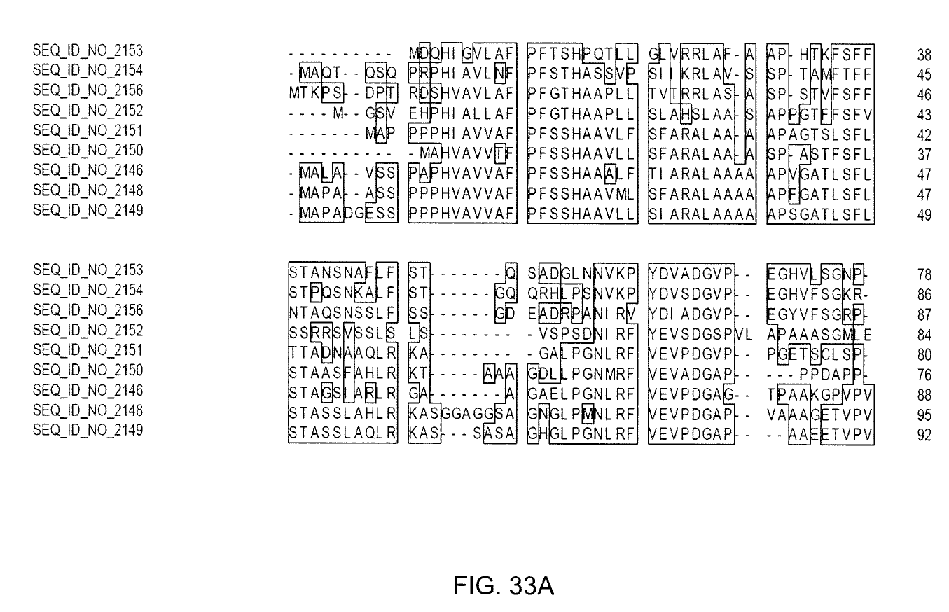

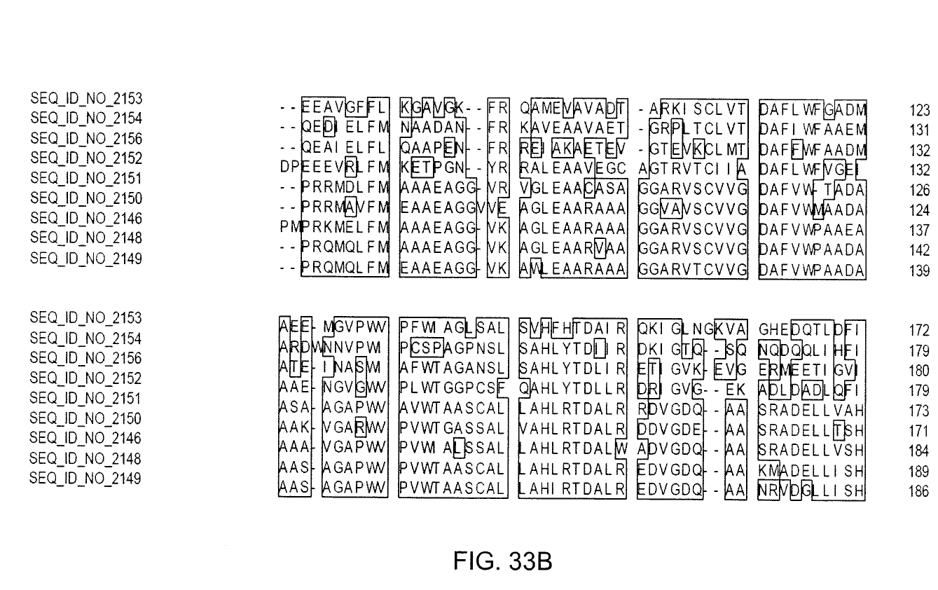

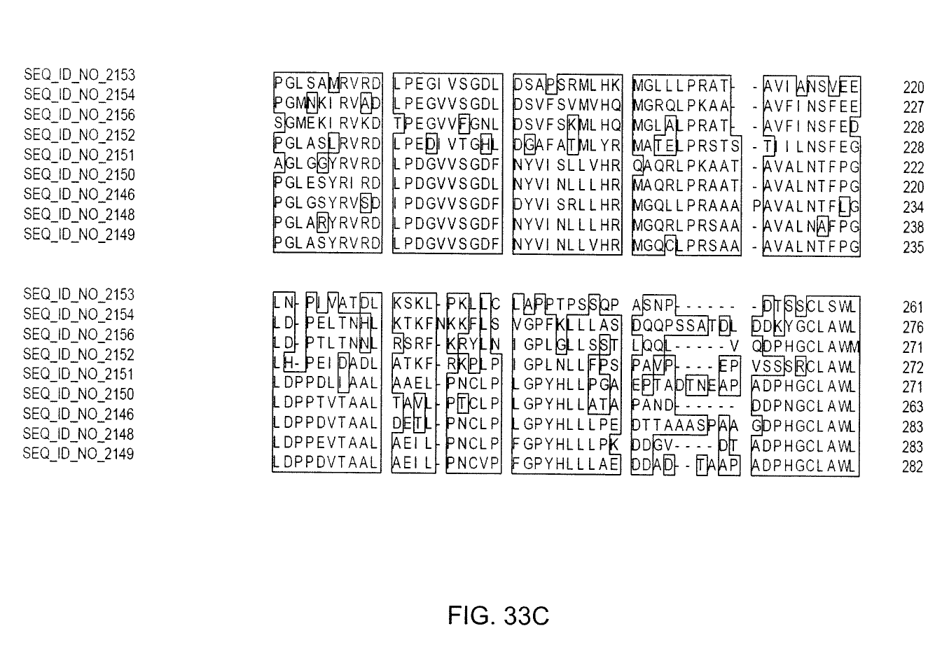

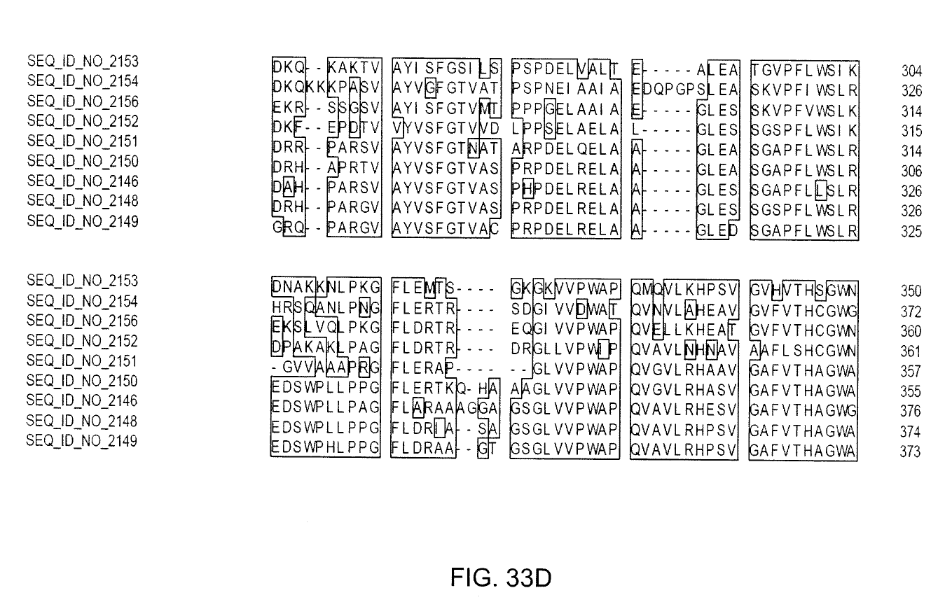

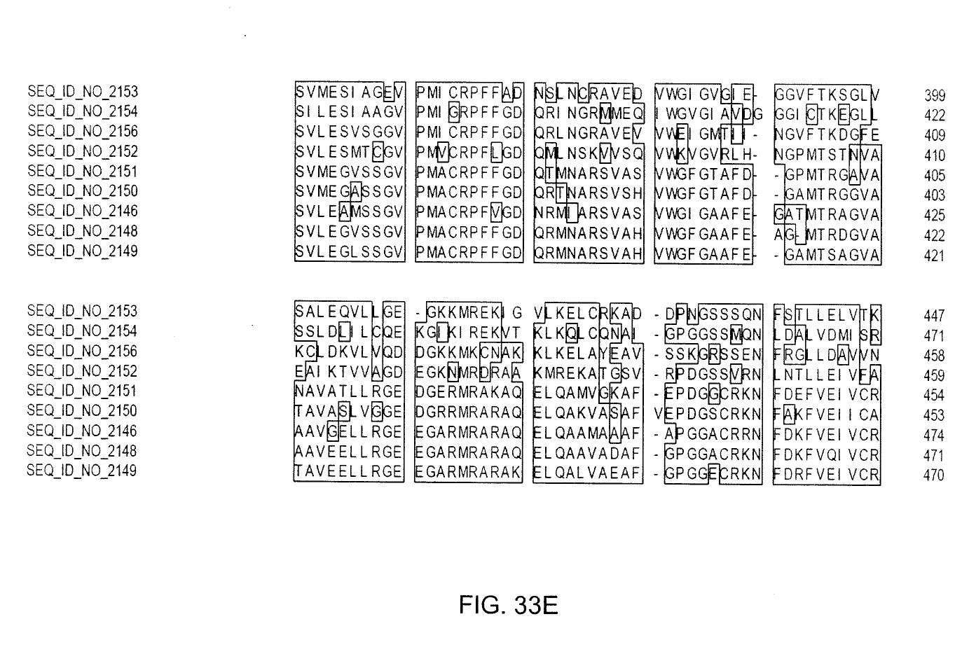

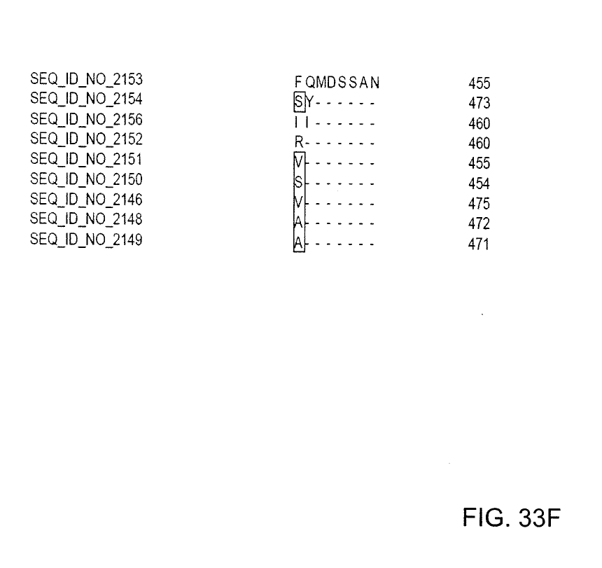

Examples of amino acid sequences of functional homologs of the polypeptide set forth in SEQ ID NO:2146 are provided in FIG. 33 and in the Sequence Listing. Such functional homologs include, for example, CeresAnnot:8735532 (SEQ ID NO:2148), GI:162463323 (SEQ ID NO:2149), GI:115466870 (SEQ ID NO:2150), GI:136746 (SEQ ID NO:2151), GI:57157447 (SEQ ID NO:2152), GI:225435965 (SEQ ID NO:2153), GI:77732554 (SEQ ID NO:2154), CeresAnnot:882862 (SEQ ID NO:2156), CeresClone:1769212 (SEQ ID NO:2158), GI:104303700 (SEQ ID NO:2159), GI:136745 (SEQ ID NO:2160), GI:104303696 (SEQ ID NO:2161), GI:104303698 (SEQ ID NO:2162), GI:55741074 (SEQ ID NO:2163), CeresClone:1900402 (SEQ ID NO:2165), CeresAnnot:8656644 (SEQ ID NO:2167), CeresClone:100743430 (SEQ ID NO:2169), GI:125557238 (SEQ ID NO:2170), GI:115470585 (SEQ ID NO:2171), GI:125554377 (SEQ ID NO:2172), GI:13620855 (SEQ ID NO:2173), GI:13620861 (SEQ ID NO:2174), GI:13620869 (SEQ ID NO:2175), GI:149350038 (SEQ ID NO:2176), GI:13620857 (SEQ ID NO:2177), GI:40645337 (SEQ ID NO:2178), GI:133874214 (SEQ ID NO:2179), CeresAnnot:1486671 (SEQ ID NO:2181), CeresClone:1851632 (SEQ ID NO:2183), GI:133874216 (SEQ ID NO:2184), CeresAnnot:1540082 (SEQ ID NO:2186), GI:51872677 (SEQ ID NO:2187), or GI:157343279 (SEQ ID NO:2188). In some cases, a functional homolog of SEQ ID NO:2146 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2146.

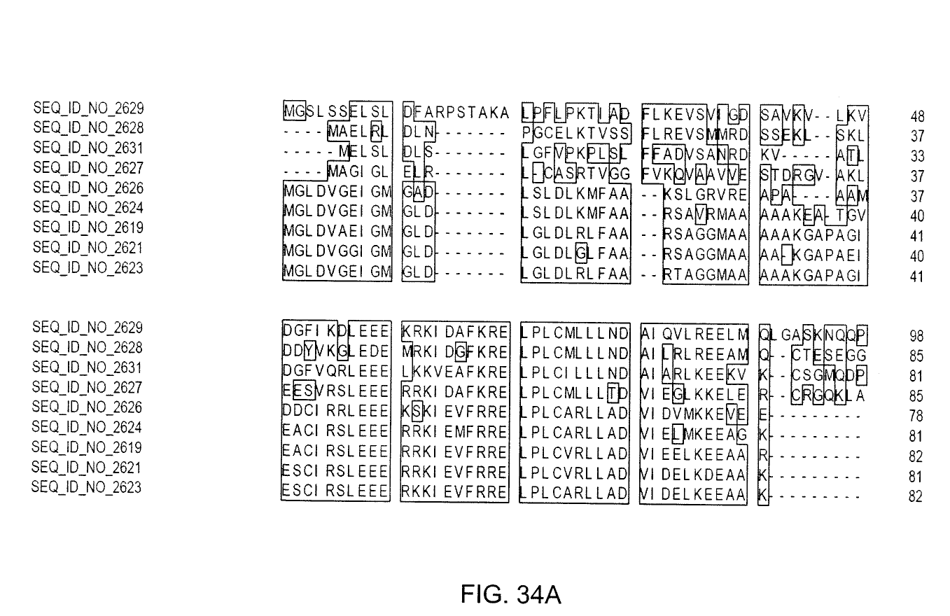

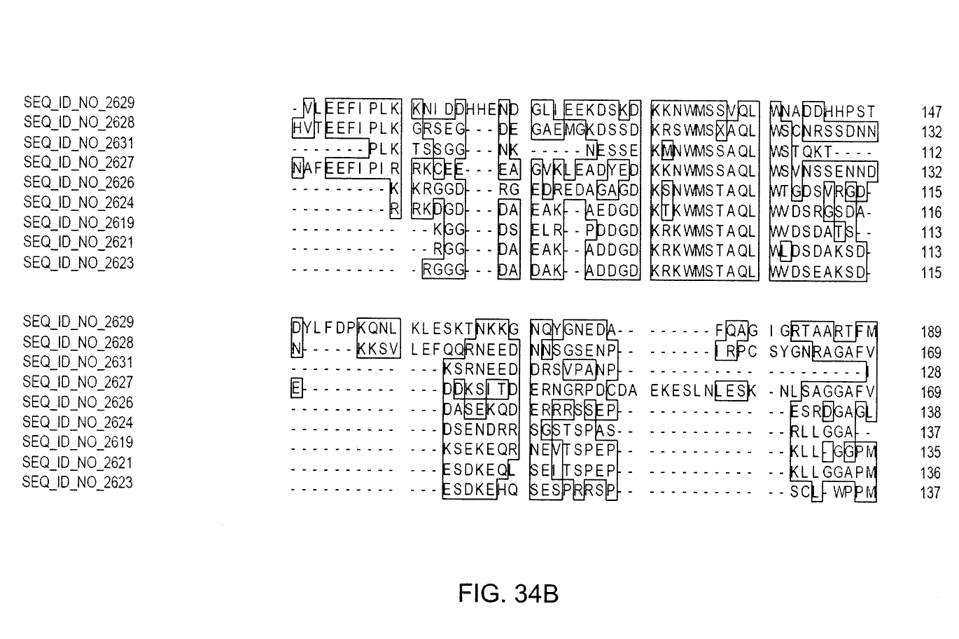

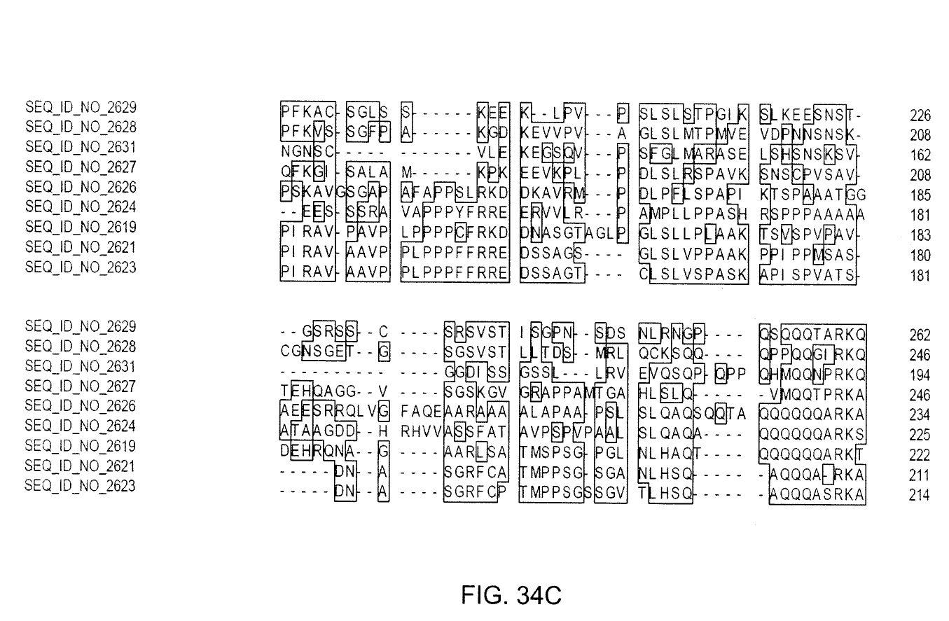

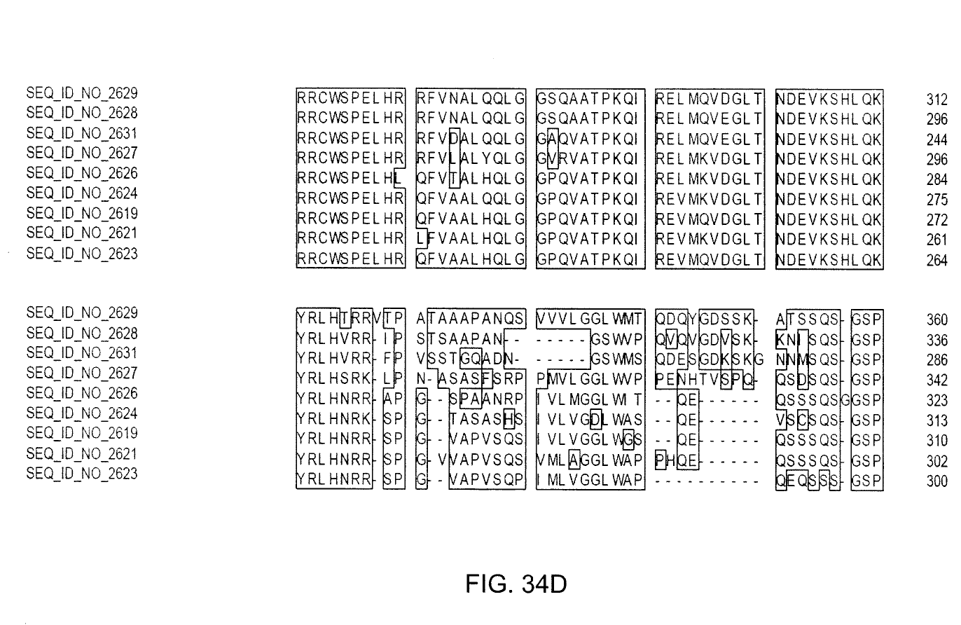

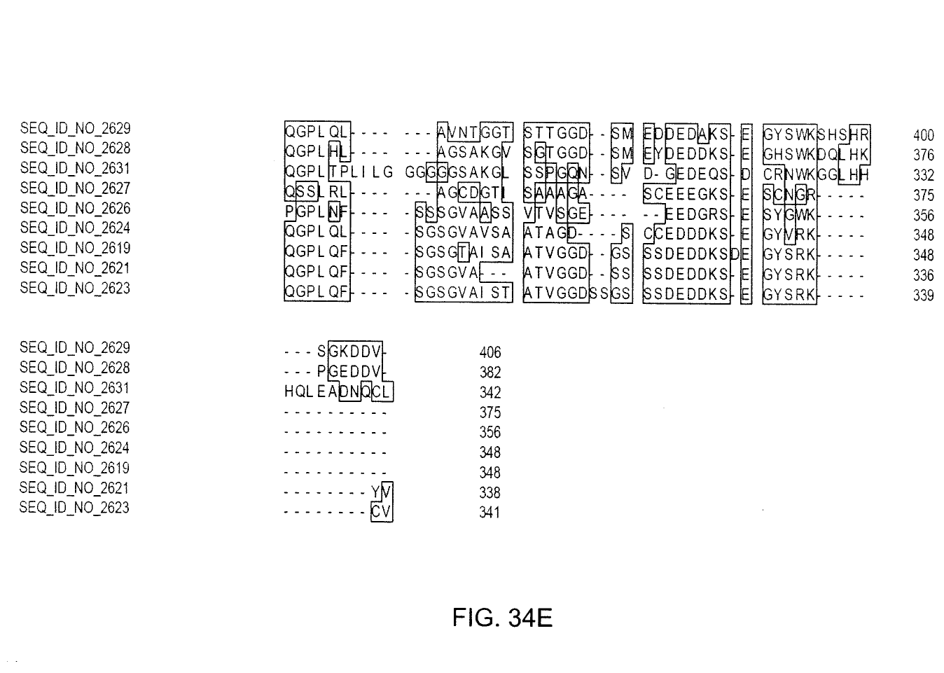

Examples of amino acid sequences of functional homologs of the polypeptide set forth in SEQ ID NO:2619 are provided in FIG. 34 and in the Sequence Listing. Such functional homologs include, for example, CeresClone:240112 (SEQ ID NO:2621), CeresAnnot:8632546 (SEQ ID NO:2623), GI:115455537 (SEQ ID NO:2624), CeresClone:630408 (SEQ ID NO:2626), GI:102139855 (SEQ ID NO:2627), GI:147860059 (SEQ ID NO:2628), GI:118486821 (SEQ ID NO:2629), CeresClone:1104471 (SEQ ID NO:2631), GI:125588020 (SEQ ID NO:2632), CeresClone:537690 (SEQ ID NO:2634), CeresClone:1828957 (SEQ ID NO:2636), CeresAnnot:8644036 (SEQ ID NO:2638), CeresClone:285434 (SEQ ID NO:2640), GI:115470343 (SEQ ID NO:2641), GI:125557053 (SEQ ID NO:2642), CeresClone:225486 (SEQ ID NO:2644), CeresAnnot:1733246 (SEQ ID NO:2646), GI:147858322 (SEQ ID NO:2647), GI:115489338 (SEQ ID NO:2648), GI:125537206 (SEQ ID NO:2649), GI:157340951 (SEQ ID NO:2650), or CeresClone:560820 (SEQ ID NO:2652). In some cases, a functional homolog of SEQ ID NO:2619 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2619.

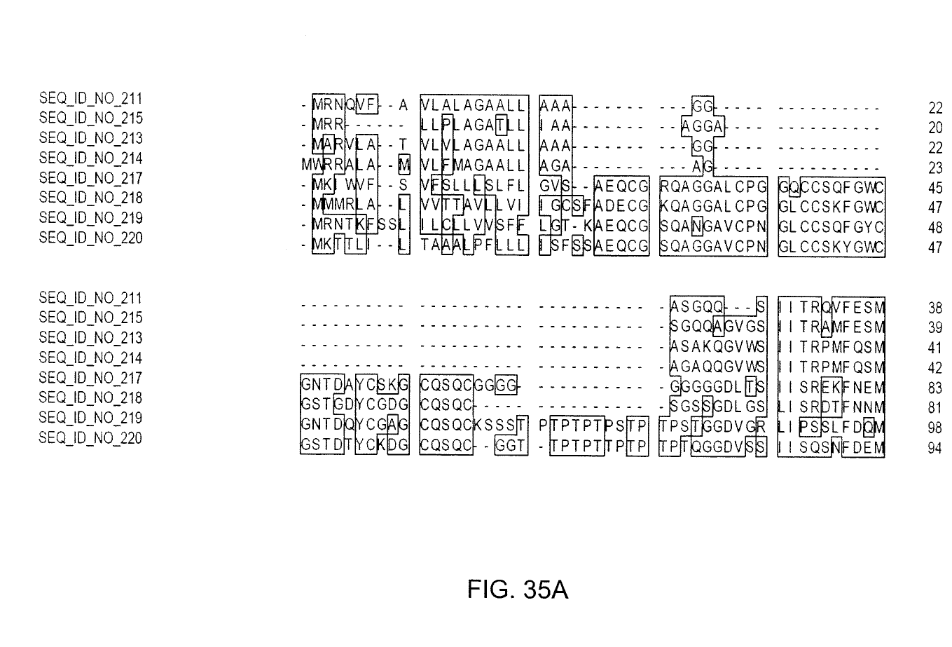

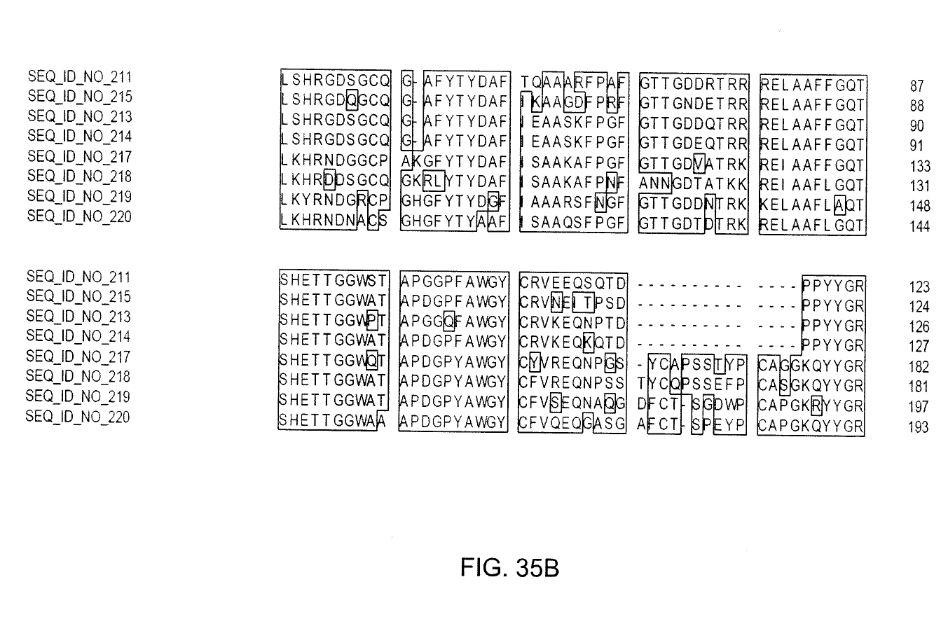

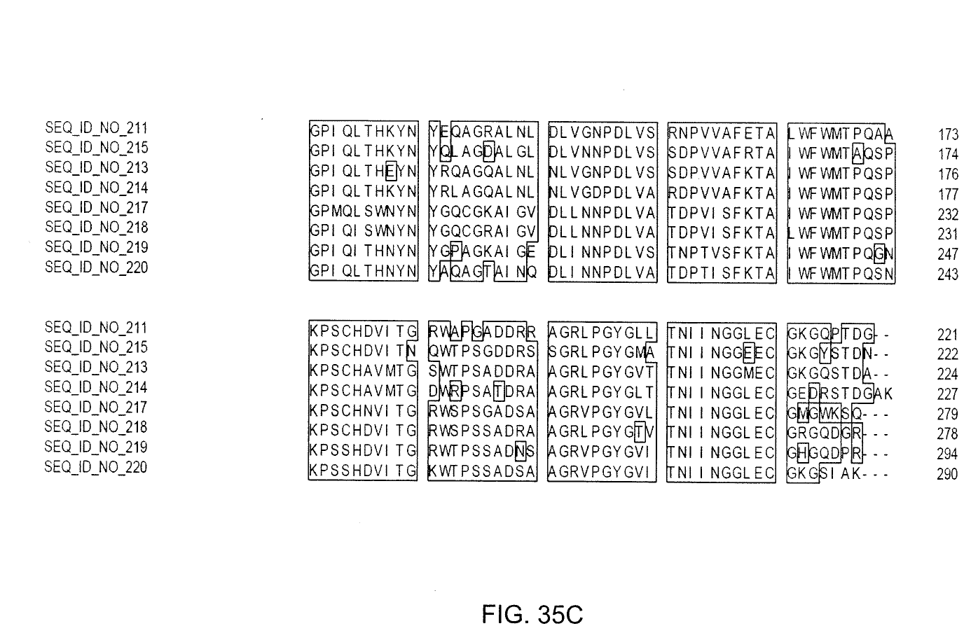

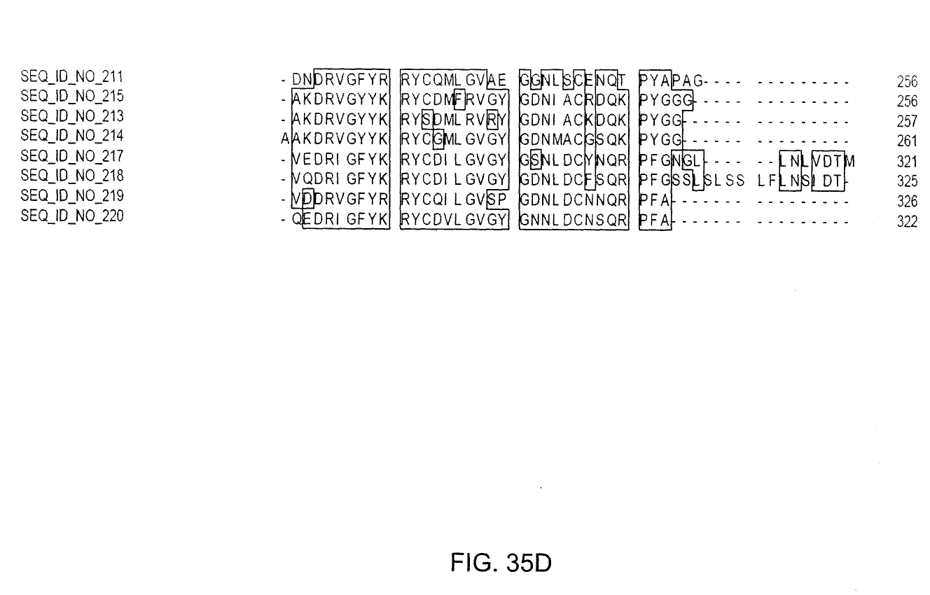

Examples of amino acid sequences of functional homologs of the polypeptide set forth in SEQ ID NO:211 are provided in FIG. 35 and in the Sequence Listing. Such functional homologs include, for example, CeresAnnot:8643337 (SEQ ID NO:213), GI:212275494 (SEQ ID NO:214), GI:115450541 (SEQ ID NO:215), CeresAnnot:1450642 (SEQ ID NO:217), GI:1781042 (SEQ ID NO:218), GI:33414050 (SEQ ID NO:219), GI:90287922 (SEQ ID NO:220), CeresClone:1672778 (SEQ ID NO:222), GI:1800141 (SEQ ID NO:223), GI:3892724 (SEQ ID NO:224), CeresClone:1319431 (SEQ ID NO:226), CeresClone:1074387 (SEQ ID NO:228), GI:544015 (SEQ ID NO:229), CeresAnnot:1513807 (SEQ ID NO:231), GI:190588358 (SEQ ID NO:232), GI:17932712 (SEQ ID NO:233), GI:4960049 (SEQ ID NO:234), GI:33414052 (SEQ ID NO:235), GI:30844174 (SEQ ID NO:236), GI:116349 (SEQ ID NO:237), GI:6573210 (SEQ ID NO:238), GI:116314 (SEQ ID NO:239), GI:584928 (SEQ ID NO:240), CeresClone:1851776 (SEQ ID NO:242), CeresClone:862325 (SEQ ID NO:244), GI:67867096 (SEQ ID NO:245), GI:116321 (SEQ ID NO:246), GI:11558417 (SEQ ID NO:247), GI:3126965 (SEQ ID NO:248), GI:10880381 (SEQ ID NO:249), GI:256133 (SEQ ID NO:250), GI:1705807 (SEQ ID NO:251), GI:30844172 (SEQ ID NO:252), CeresClone:705065 (SEQ ID NO:254), GI:38261495 (SEQ ID NO:255), GI:17932710 (SEQ ID NO:256), GI:125552257 (SEQ ID NO:257), GI:48237775 (SEQ ID NO:258), GI:38371990 (SEQ ID NO:259), GI:1705813 (SEQ ID NO:260), GI:1354129 (SEQ ID NO:261), GI:561873 (SEQ ID NO:262), GI:3201547 (SEQ ID NO:263), GI:89242716 (SEQ ID NO:264), GI:115470054 (SEQ ID NO:265), GI:17028128 (SEQ ID NO:266), GI:227845 (SEQ ID NO:267), GI:48237771 (SEQ ID NO:268), GI:115463755 (SEQ ID NO:269), GI:157328752 (SEQ ID NO:270), GI:118489654 (SEQ ID NO:271), GI:88659025 (SEQ ID NO:272), GI:147805840 (SEQ ID NO:273), GI:75309544 (SEQ ID NO:274), GI:89275293 (SEQ ID NO:275), GI:407472 (SEQ ID NO:276), GI:10047221 (SEQ ID NO:277), GI:115463753 (SEQ ID NO:278), GI:169990894 (SEQ ID NO:279), CeresClone:2022546 (SEQ ID NO:281), or GI:116786735 (SEQ ID NO:282) In some cases, a functional homolog of SEQ ID NO:211 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:211.

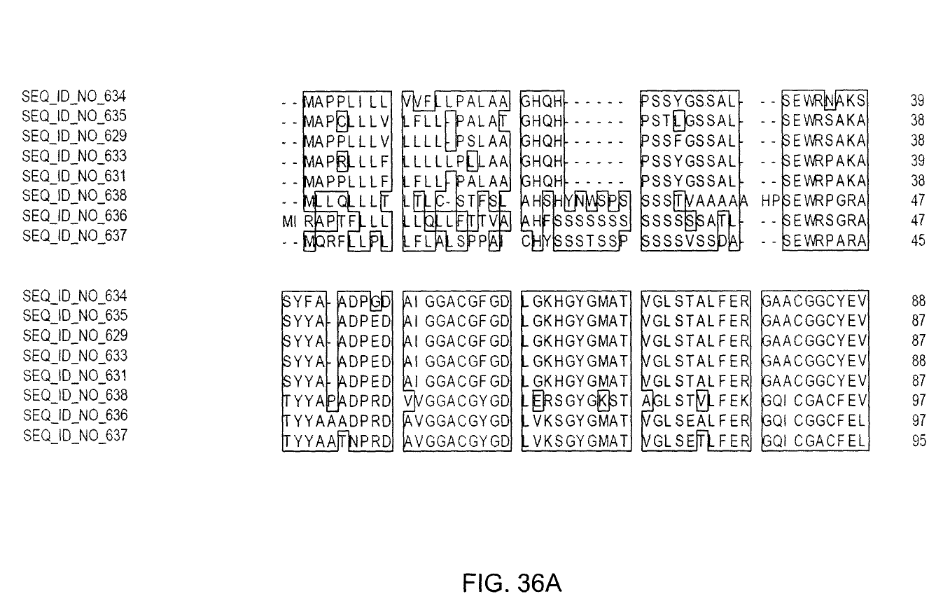

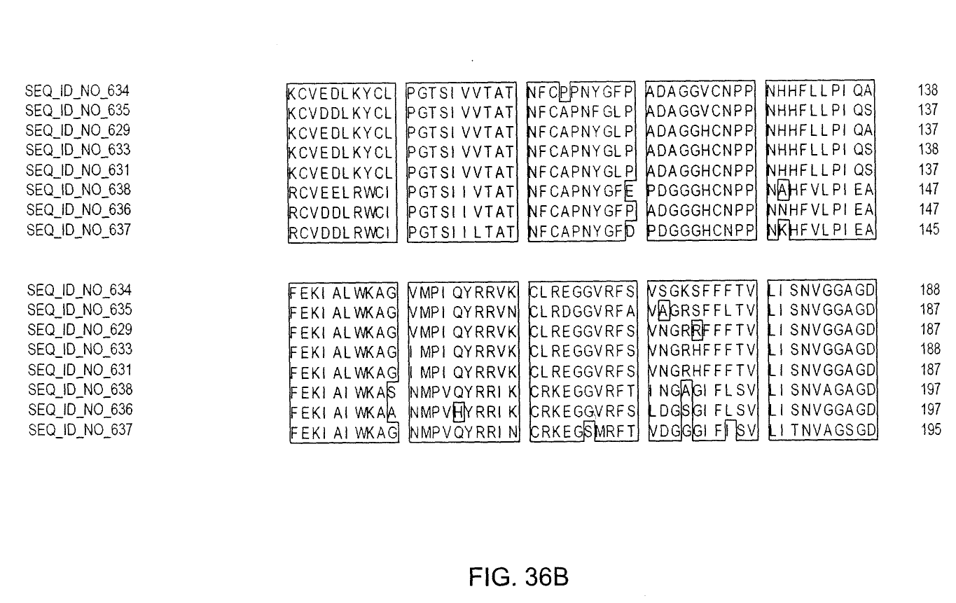

Examples of amino acid sequences of functional homologs of the polypeptide set forth in SEQ ID NO:629 are provided in FIG. 36 and in the Sequence Listing. Such functional homologs include, for example, CeresAnnot:8702536 (SEQ ID NO:631), CeresClone:222556 (SEQ ID NO:633), GI:44894790 (SEQ ID NO:634), GI:115460126 (SEQ ID NO:635), GI:157347738 (SEQ ID NO:636), GI:18396479 (SEQ ID NO:637), GI:4138912 (SEQ ID NO:638), CeresClone:1722246 (SEQ ID NO:640), CeresClone:100892266 (SEQ ID NO:642), CeresClone:683350 (SEQ ID NO:644), CeresClone:659599 (SEQ ID NO:646), CeresClone:1793544 (SEQ ID NO:648), CeresAnnot:1449519 (SEQ ID NO:650), GI:6714416 (SEQ ID NO:651), CeresAnnot:1487289 (SEQ ID NO:653), GI:29467505 (SEQ ID NO:654), GI:147712812 (SEQ ID NO:655), GI:29467507 (SEQ ID NO:656), GI:29466641 (SEQ ID NO:657), GI:17484121 (SEQ ID NO:658), GI:10180019 (SEQ ID NO:659), GI:38046730 (SEQ ID NO:660), CeresAnnot:1464450 (SEQ ID NO:662), GI:225464253 (SEQ ID NO:663), GI:20502788 (SEQ ID NO:664), GI:6942322 (SEQ ID NO:665), GI:1040875 (SEQ ID NO:666), or GI:167860796 (SEQ ID NO:667). In some cases, a functional homolog of SEQ ID NO:629 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:629.

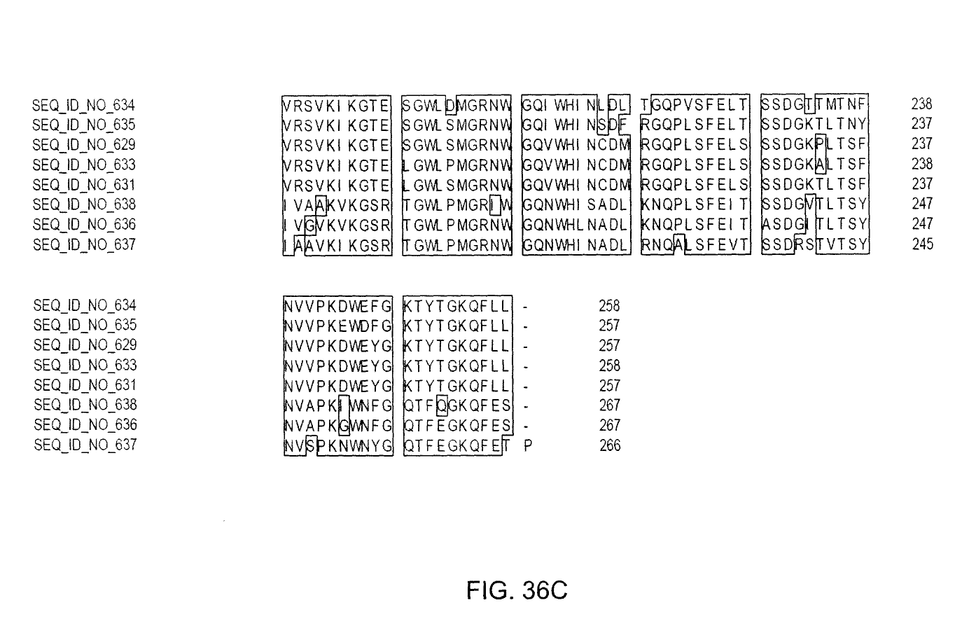

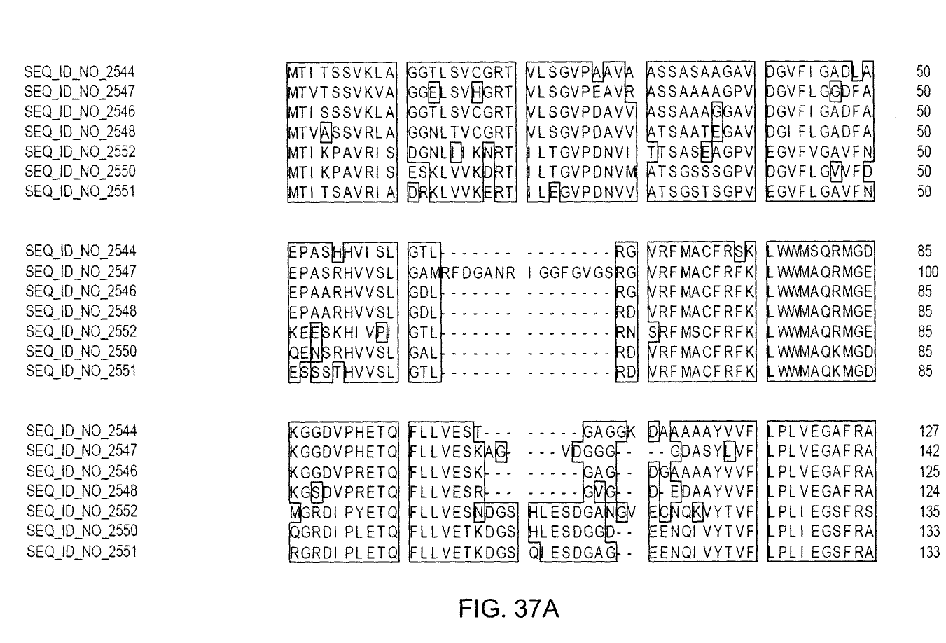

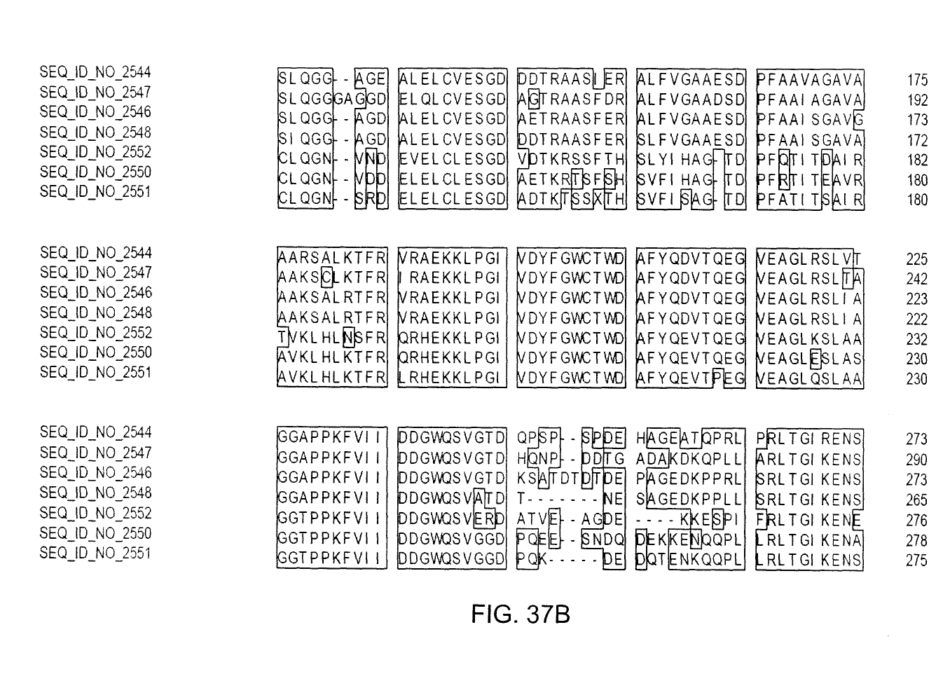

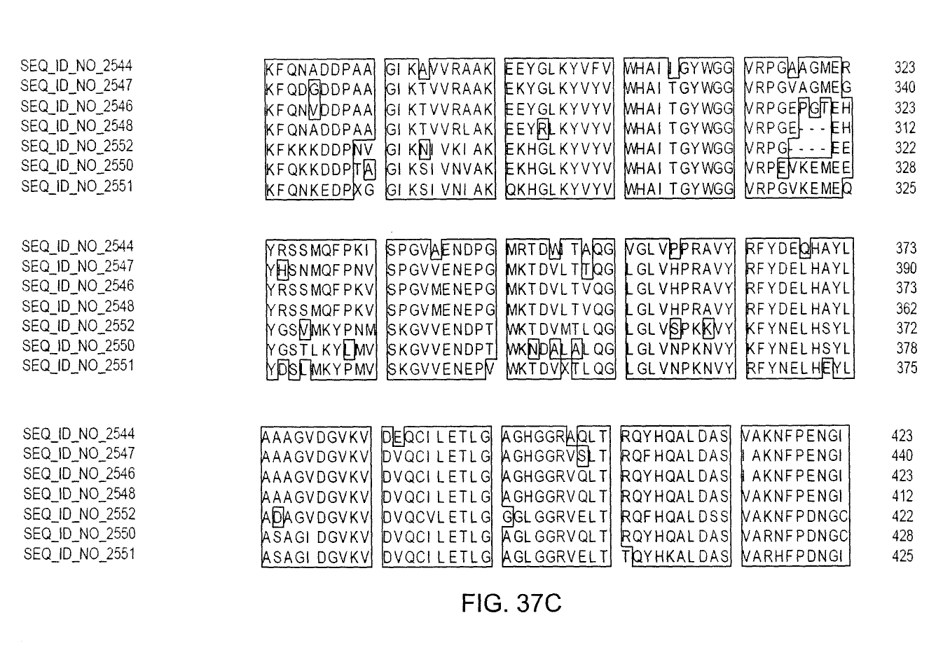

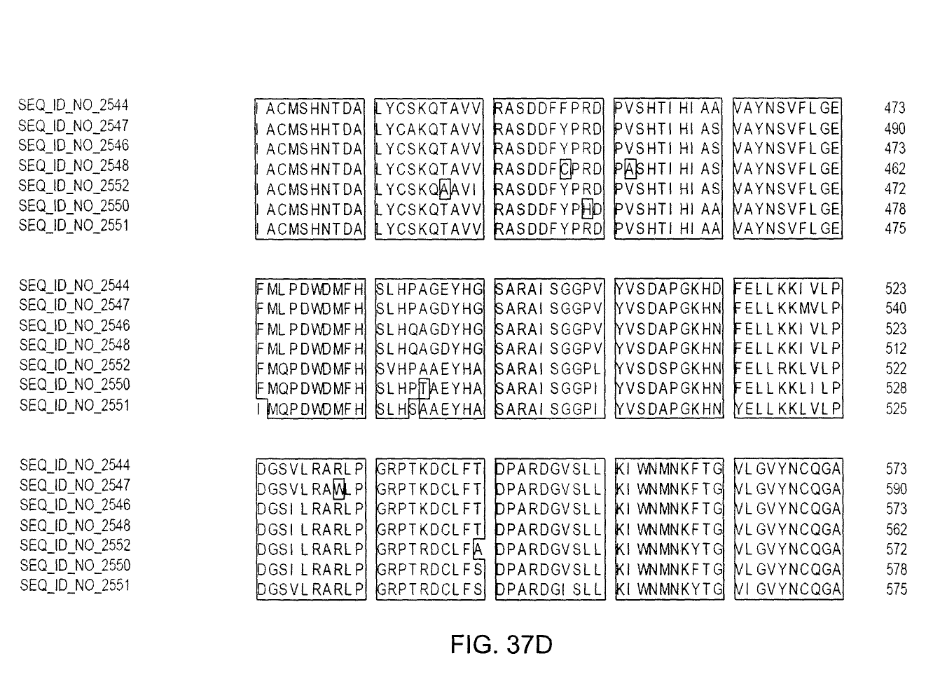

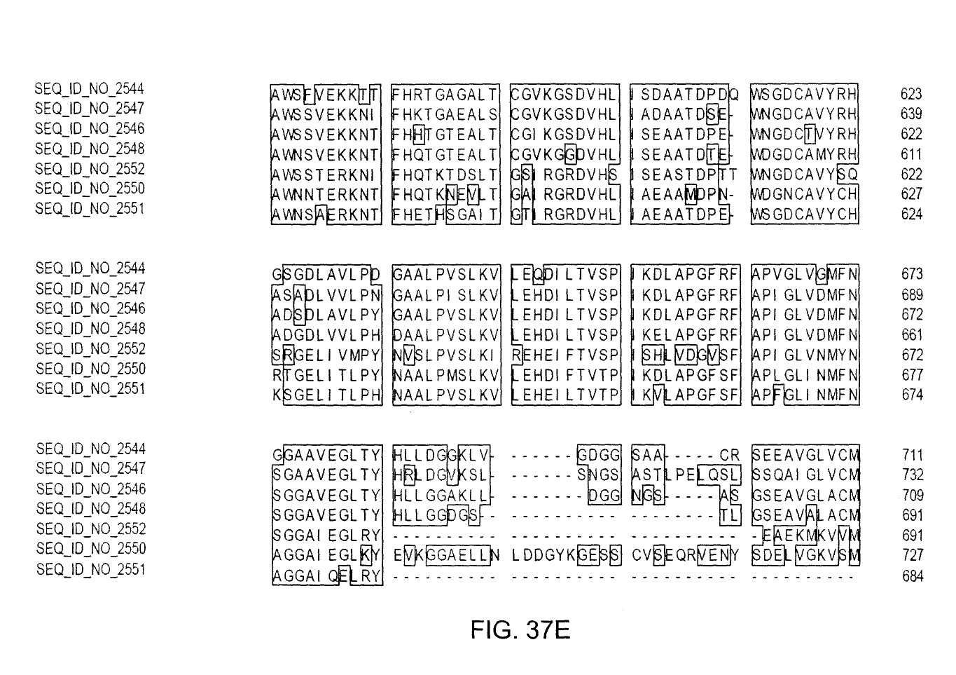

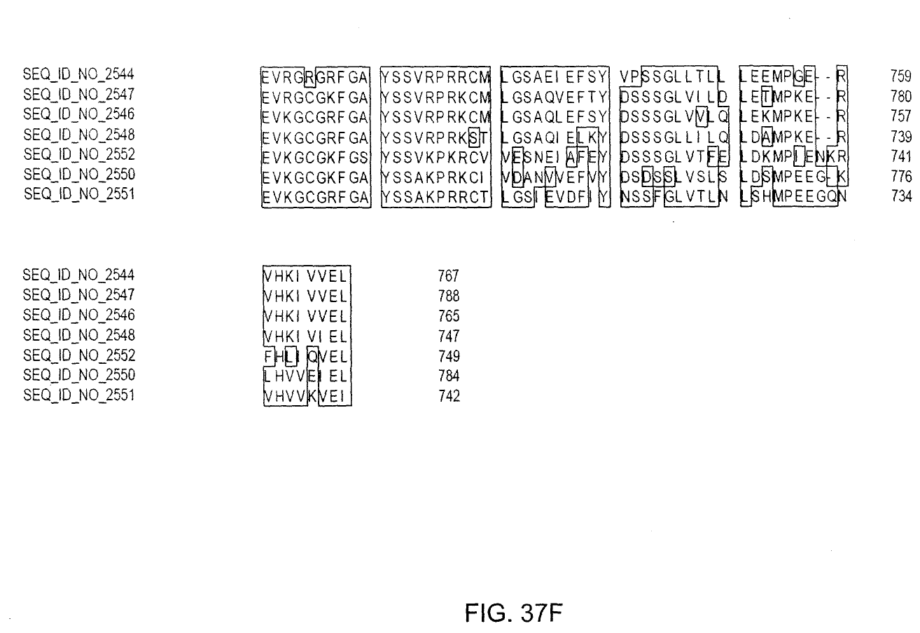

Examples of amino acid sequences of functional homologs of the polypeptide set forth in SEQ ID NO:2544 are provided in FIG. 37 and in the Sequence Listing. Such functional homologs include, for example, CeresClone:1806608 (SEQ ID NO:2544), CeresAnnot:8735307 (SEQ ID NO:2546), GI:115466686 (SEQ ID NO:2547), GI:162462869 (SEQ ID NO:2548), CeresAnnot:1478531 (SEQ ID NO:2550), GI:147865342 (SEQ ID NO:2551), GI:15241300 (SEQ ID NO:2552), CeresAnnot:1453769 (SEQ ID NO:2554), CeresAnnot:1532021 (SEQ ID NO:2556), GI:157343006 (SEQ ID NO:2557), GI:50540754 (SEQ ID NO:2558), GI:125546144 (SEQ ID NO:2559), CeresAnnot:8632040 (SEQ ID NO:2561), GI:167100 (SEQ ID NO:2562), CeresClone:1807161 (SEQ ID NO:2564), GI:157348674 (SEQ ID NO:2565), CeresAnnot:8645125 (SEQ ID NO:2567), CeresClone:1533376 (SEQ ID NO:2569), CeresAnnot:1453705 (SEQ ID NO:2571), GI:125557655 (SEQ ID NO:2572), GI:29838631 (SEQ ID NO:2573), GI:115471135 (SEQ ID NO:2574), GI:488787 (SEQ ID NO:2575), GI:91075914 (SEQ ID NO:2576), CeresAnnot:1474940 (SEQ ID NO:2578), CeresAnnot:875520 (SEQ ID NO:2580), GI:7242785 (SEQ ID NO:2581), CeresAnnot:1528375 (SEQ ID NO:2583), GI:148925503 (SEQ ID NO:2584), GI:42408863 (SEQ ID NO:2585), CeresAnnot:1480477 (SEQ ID NO:2587), GI:23452226 (SEQ ID NO:2588), GI:29838629 (SEQ ID NO:2589), GI:157353289 (SEQ ID NO:2590), CeresClone:927786 (SEQ ID NO:2592), GI:87128422 (SEQ ID NO:2593), GI:168002098 (SEQ ID NO:2594), GI:15222768 (SEQ ID NO:2595), or CeresClone:1768954 (SEQ ID NO:2597). In some cases, a functional homolog of SEQ ID NO:2544 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2544.

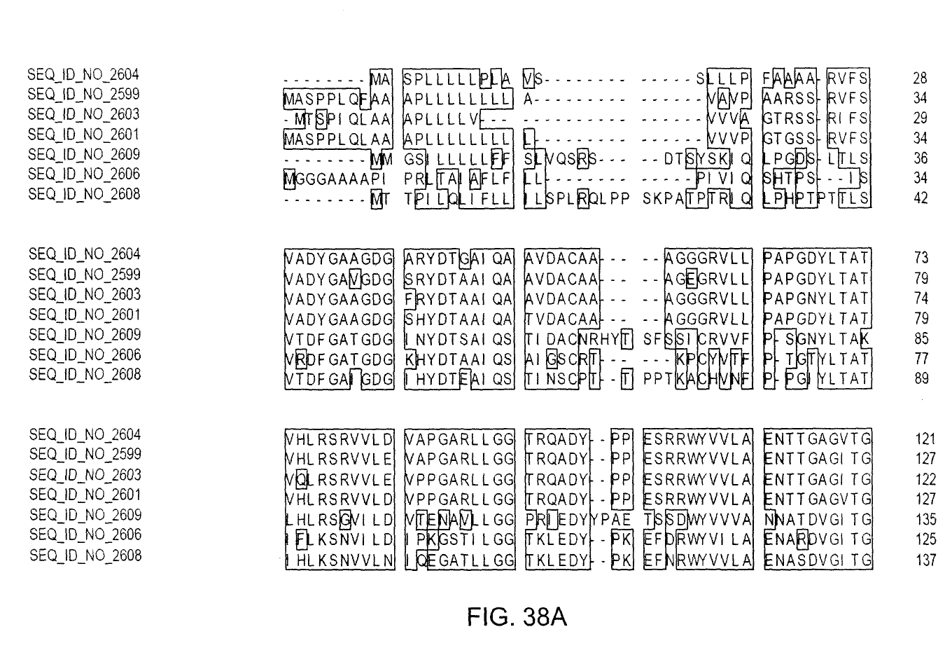

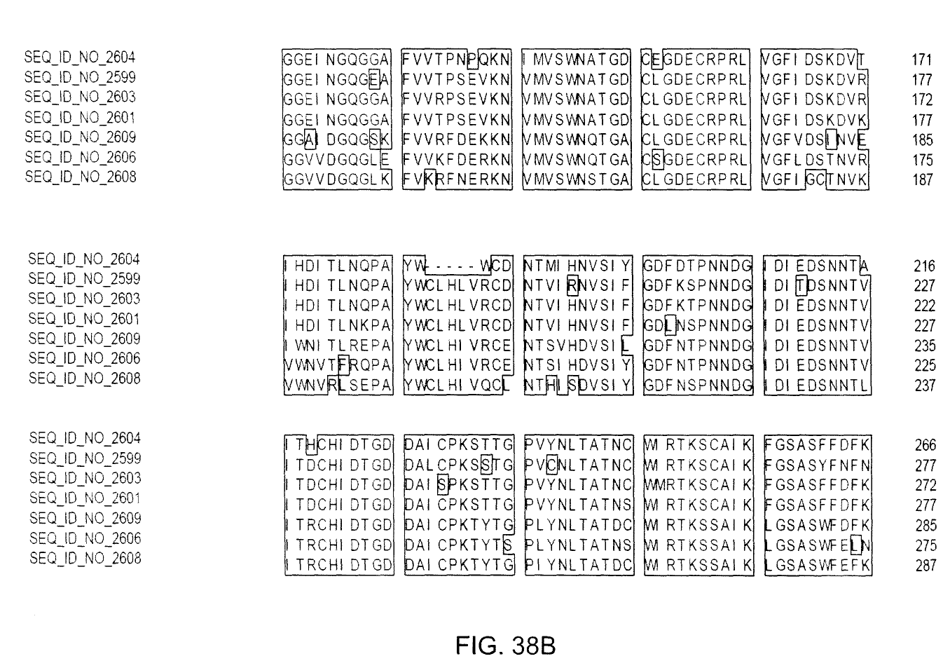

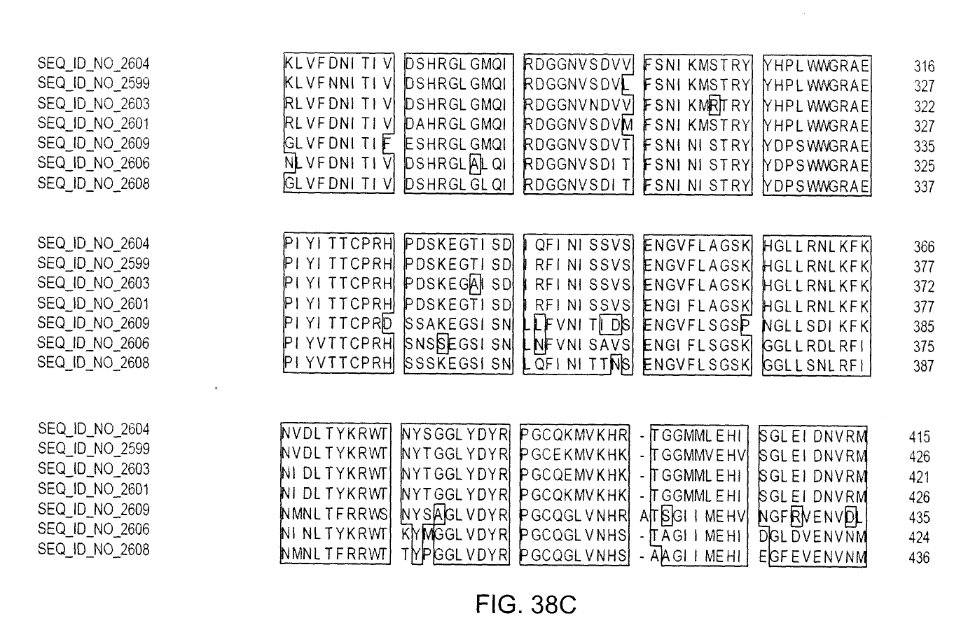

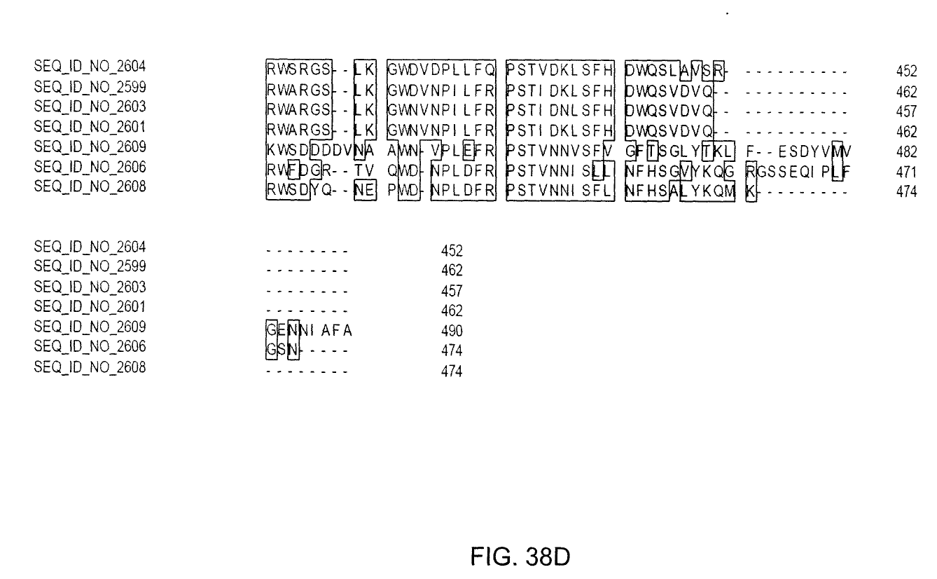

Examples of amino acid sequences of functional homologs of the polypeptide set forth in SEQ ID NO:2599 are provided in FIG. 38 and in the Sequence Listing. Such functional homologs include, for example, CeresAnnot:8654883 (SEQ ID NO:2601), CeresClone:236513 (SEQ ID NO:2603), GI:115479865 (SEQ ID NO:2604), CeresClone:1857333 (SEQ ID NO:2606), CeresAnnot:1528328 (SEQ ID NO:2608), GI:18410809 (SEQ ID NO:2609), CeresClone:1905277 (SEQ ID NO:2611), GI:194697548 (SEQ ID NO:2612), CeresAnnot:1474898 (SEQ ID NO:2614), GI:168063573 (SEQ ID NO:2615), GI:157348638 (SEQ ID NO:2616), or GI:17473561 (SEQ ID NO:2617). In some cases, a functional homolog of SEQ ID NO:2599 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2599.

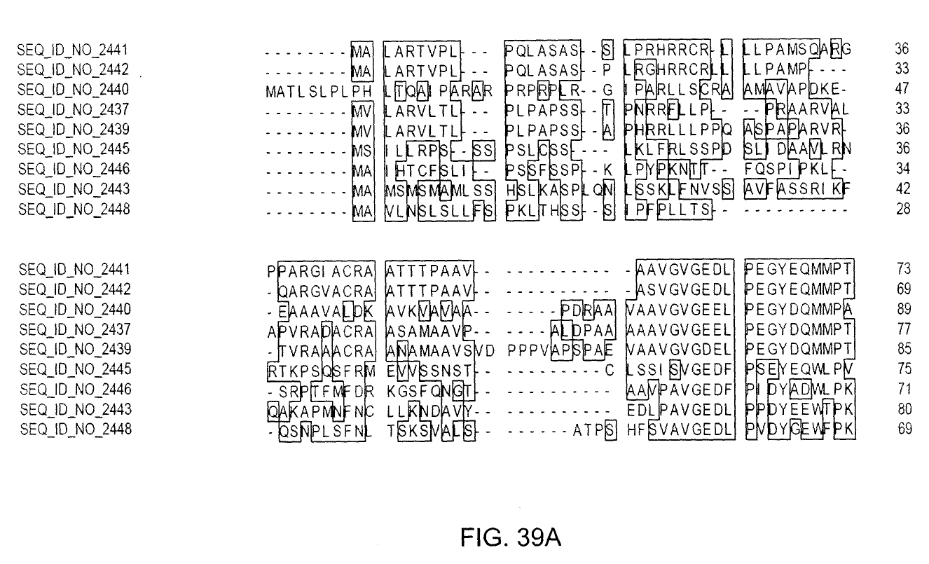

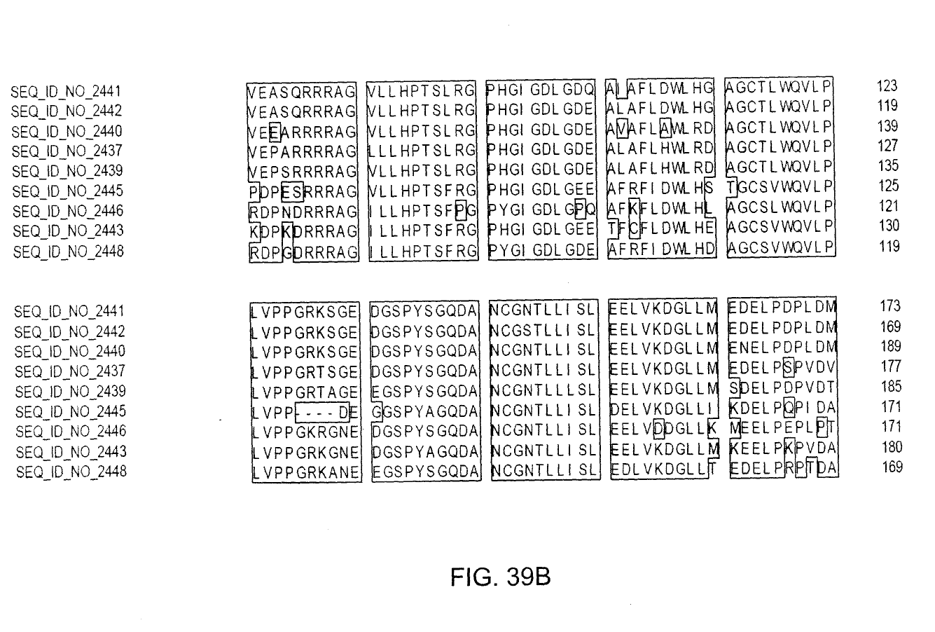

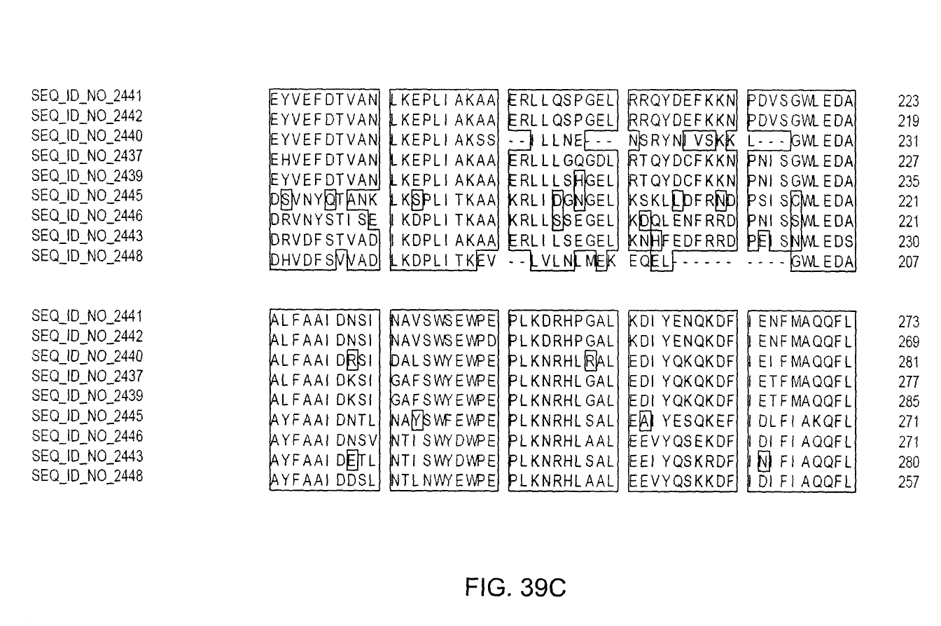

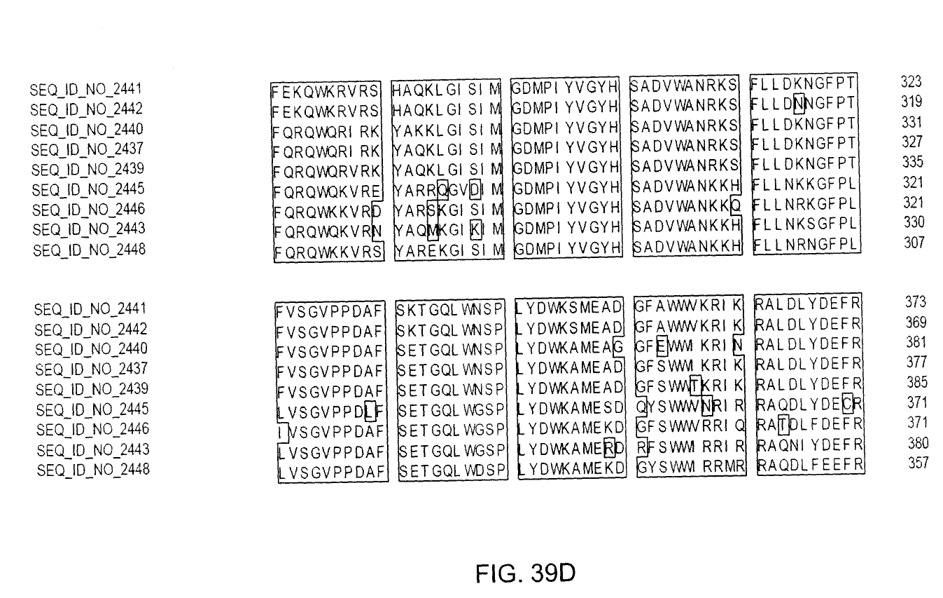

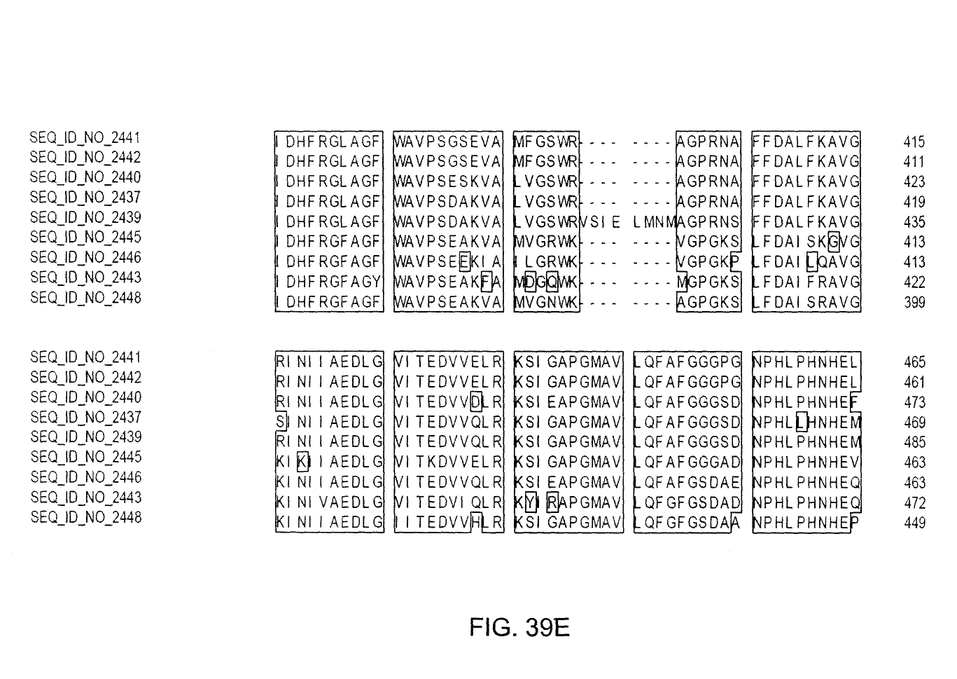

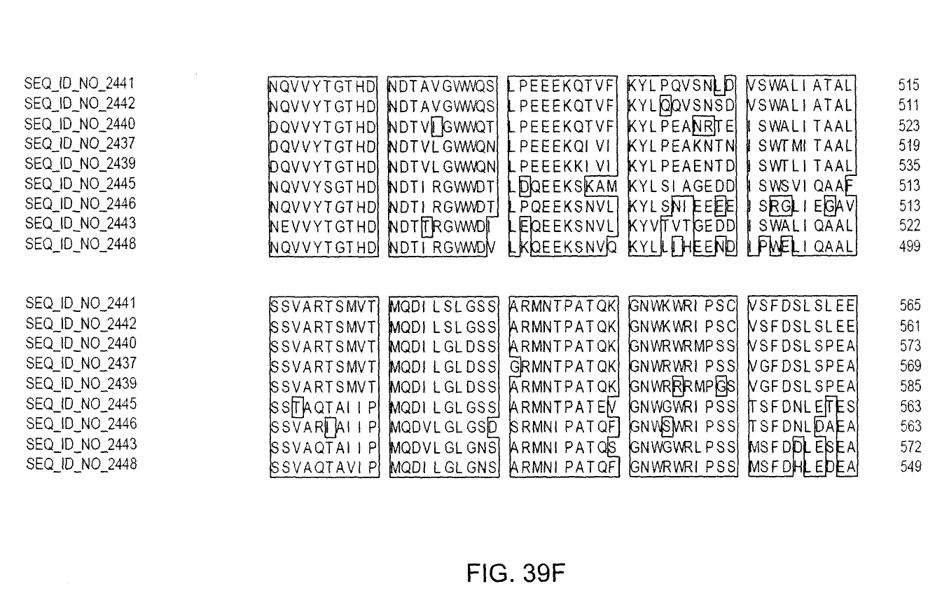

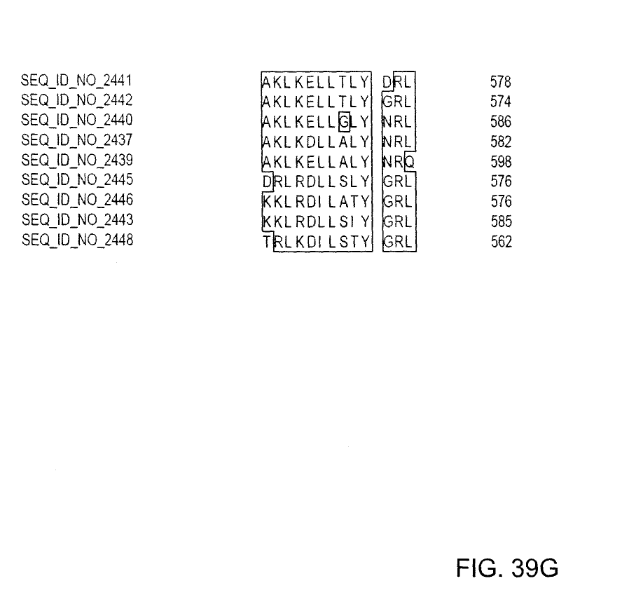

Examples of amino acid sequences of functional homologs of the polypeptide set forth in SEQ ID NO:2437 are provided in FIG. 39 and in the Sequence Listing. Such functional homologs include, for example, CeresAnnot:8656832 (SEQ ID NO:2439), GI:22093785 (SEQ ID NO:2440), GI:71084353 (SEQ ID NO:2441), GI:71084351 (SEQ ID NO:2442), GI:157345040 (SEQ ID NO:2443), CeresAnnot:575801 (SEQ ID NO:2445), GI:544184 (SEQ ID NO:2446), CeresAnnot:1509727 (SEQ ID NO:2448), GI:115473487 (SEQ ID NO:2449), CeresClone:1858312 (SEQ ID NO:2451), GI:99031627 (SEQ ID NO:2452), CeresClone:1190476 (SEQ ID NO:2454), CeresAnnot:1456535 (SEQ ID NO:2456), GI:168016526 (SEQ ID NO:2457), GI:168034311 (SEQ ID NO:2458), GI:168001012 (SEQ ID NO:2459), GI:11095335 (SEQ ID NO:2460), GI:159489864 (SEQ ID NO:2461), GI:145342068 (SEQ ID NO:2462), GI:116061732 (SEQ ID NO:2463), or GI:46360132 (SEQ ID NO:2464). In some cases, a functional homolog of SEQ ID NO:2437 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2437.

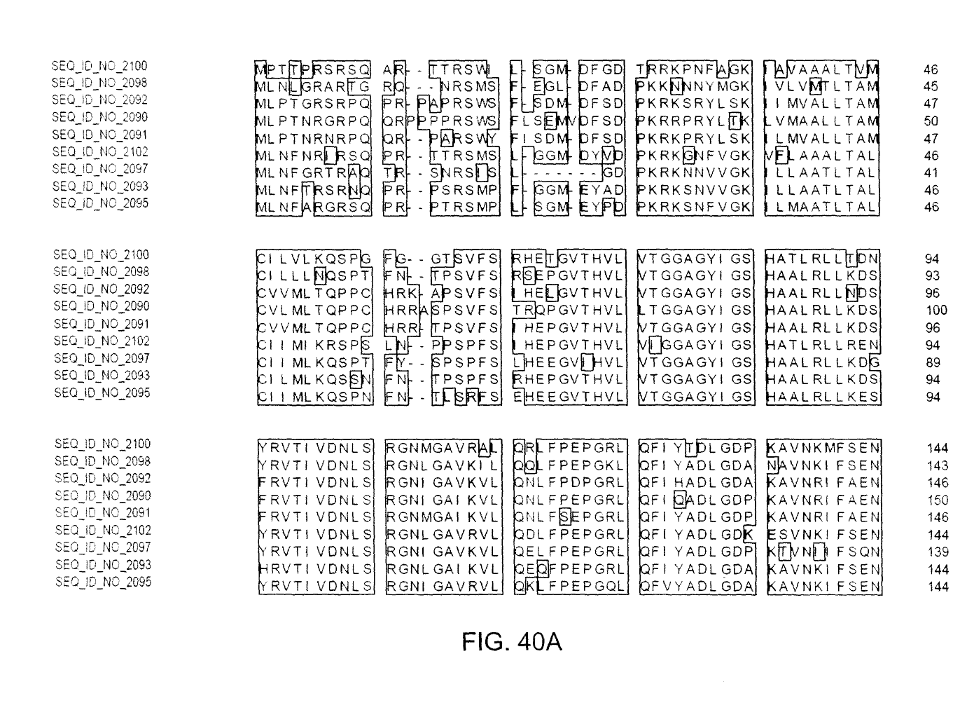

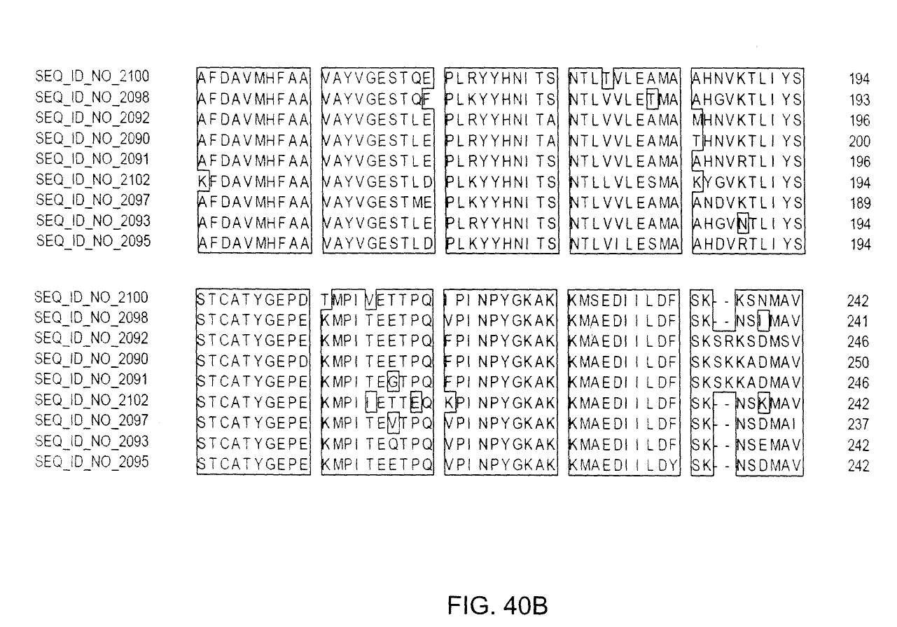

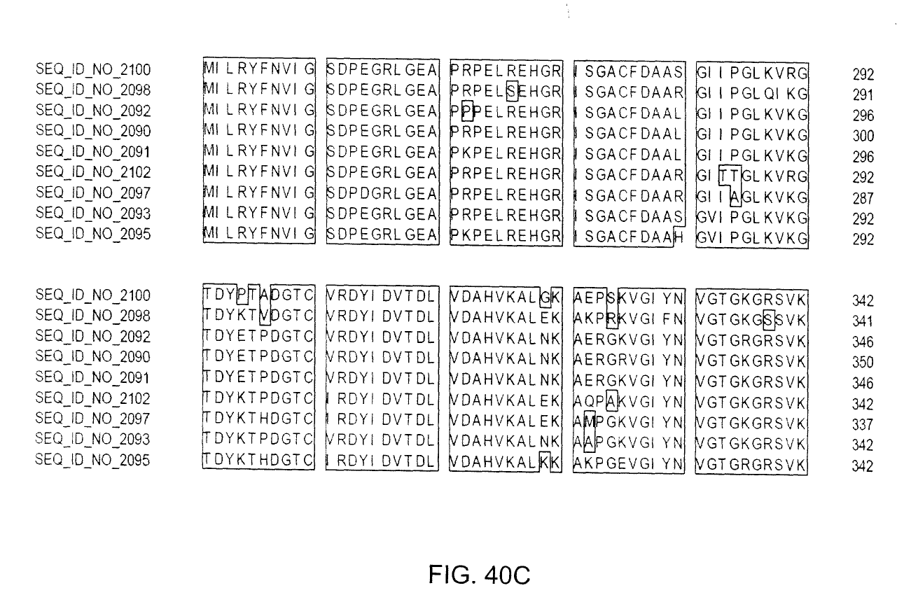

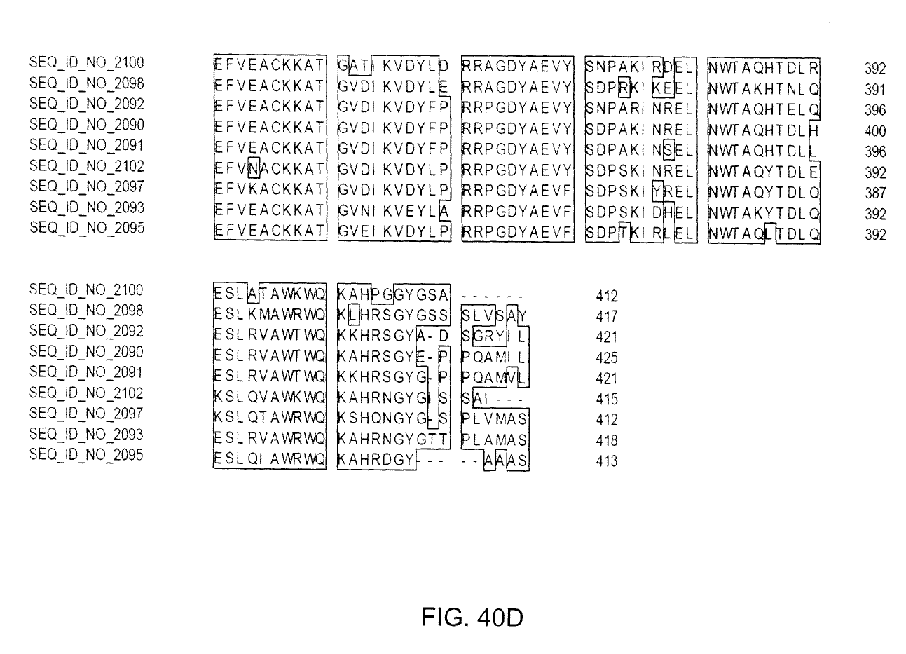

Examples of amino acid sequences of functional homologs of the polypeptide set forth in SEQ ID NO:2090 are provided in FIG. 40 and in the Sequence Listing. Such functional homologs include, for example, GI:115470521 (SEQ ID NO:2091), GI:84873492 (SEQ ID NO:2092), GI:157351651 (SEQ ID NO:2093), CeresClone:1971052 (SEQ ID NO:2095), CeresAnnot:1520551 (SEQ ID NO:2097), GI:93172441 (SEQ ID NO:2098), CeresClone:913922 (SEQ ID NO:2100), CeresClone:1659137 (SEQ ID NO:2102), CeresClone:2010819 (SEQ ID NO:2104), GI:25553524 (SEQ ID NO:2105), GI:27260937 (SEQ ID NO:2106), CeresClone:1754251 (SEQ ID NO:2108), CeresAnnot:8641586 (SEQ ID NO:2110), CeresClone:1985329 (SEQ ID NO:2112), GI:22329866 (SEQ ID NO:2113), CeresAnnot:8702875 (SEQ ID NO:2115), CeresAnnot:1495011 (SEQ ID NO:2117), GI:24417398 (SEQ ID NO:2118), GI:115474641 (SEQ ID NO:2119), GI:42408459 (SEQ ID NO:2120), GI:125560032 (SEQ ID NO:2121), GI:145324086 (SEQ ID NO:2122), CeresClone:522841 (SEQ ID NO:2124), CeresAnnot:1467188 (SEQ ID NO:2126), CeresClone:1396123 (SEQ ID NO:2128), GI:82592627 (SEQ ID NO:2129), GI:84873496 (SEQ ID NO:2130), CeresAnnot:1442125 (SEQ ID NO:2132), GI:157352102 (SEQ ID NO:2133), CeresAnnot:1453370 (SEQ ID NO:2135), GI:115460580 (SEQ ID NO:2136), GI:90399327 (SEQ ID NO:2137), GI:15241503 (SEQ ID NO:2138), CeresAnnot:1528021 (SEQ ID NO:2140), GI:168042311 (SEQ ID NO:2141), GI:168013276 (SEQ ID NO:2142), GI:168021237 (SEQ ID NO:2143), or GI:168056693 (SEQ ID NO:2144). In some cases, a functional homolog of SEQ ID NO:2090 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2090.

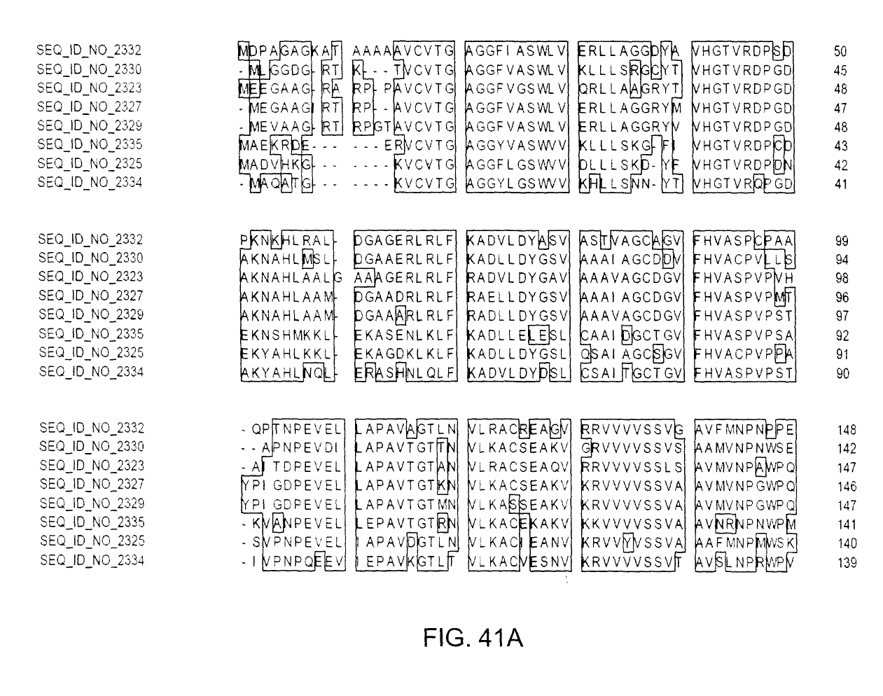

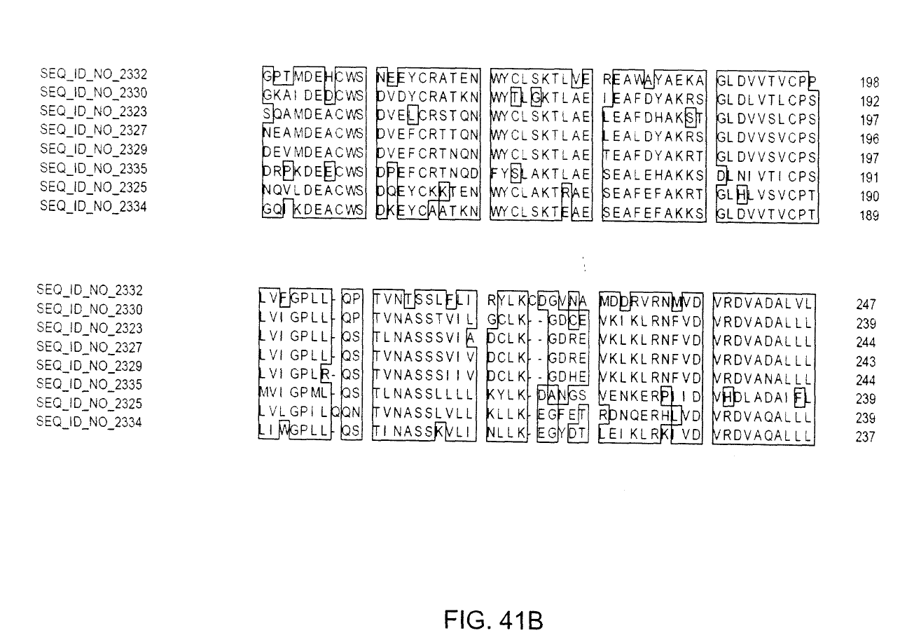

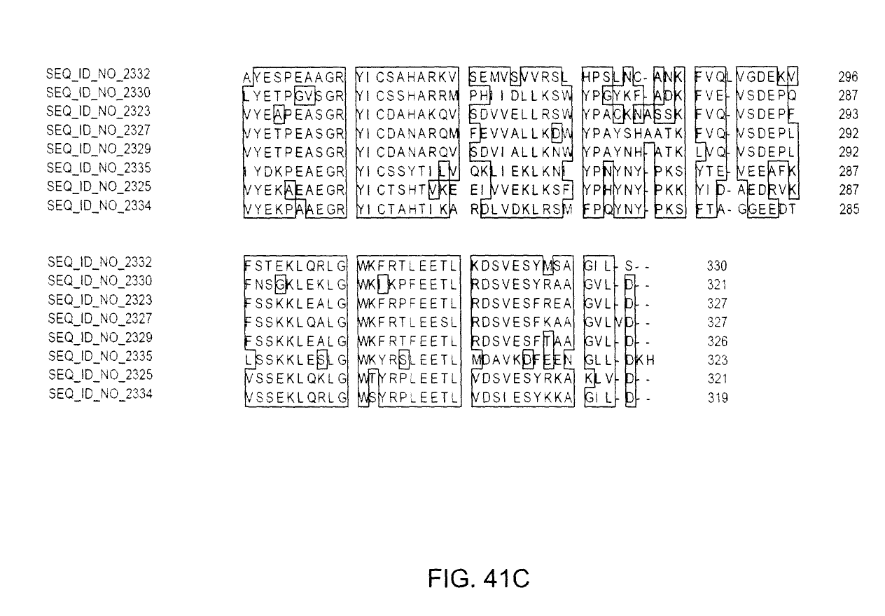

Examples of amino acid sequences of functional homologs of the polypeptide set forth in SEQ ID NO:2323 are provided in FIG. 41 and in the Sequence Listing. Such functional homologs include, for example, CeresAnnot:860791 (SEQ ID NO:2325), CeresClone:100899726 (SEQ ID NO:2327), CeresAnnot:8743558 (SEQ ID NO:2329), GI:115469012 (SEQ ID NO:2330), CeresClone:696103 (SEQ ID NO:2332), CeresClone:1930079 (SEQ ID NO:2334), GI:157350602 (SEQ ID NO:2335), CeresAnnot:8743563 (SEQ ID NO:2337), CeresAnnot:8743566 (SEQ ID NO:2339), CeresClone:677424 (SEQ ID NO:2341), CeresAnnot:8743556 (SEQ ID NO:2343), CeresAnnot:8690999 (SEQ ID NO:2345), GI:115469010 (SEQ ID NO:2346), GI:125556115 (SEQ ID NO:2347), GI:212721784 (SEQ ID NO:2348), CeresAnnot:8704900 (SEQ ID NO:2350), CeresAnnot:8743557 (SEQ ID NO:2352), CeresClone:229676 (SEQ ID NO:2354), CeresClone:336720 (SEQ ID NO:2356), GI:194707432 (SEQ ID NO:2357), GI:15226135 (SEQ ID NO:2358), CeresAnnot:8462178 (SEQ ID NO:2360), GI:157358613 (SEQ ID NO:2361), GI:125560374 (SEQ ID NO:2362), CeresClone:477792 (SEQ ID NO:2364), CeresClone:1824941 (SEQ ID NO:2366), GI:157344669 (SEQ ID NO:2367), CeresAnnot:1506214 (SEQ ID NO:2369), CeresAnnot:1442360 (SEQ ID NO:2371), CeresClone:1790816 (SEQ ID NO:2373), CeresClone:1536779 (SEQ ID NO:2375), GI:157344658 (SEQ ID NO:2376), CeresClone:303165 (SEQ ID NO:2378), CeresClone:2008188 (SEQ ID NO:2380), GI:145337634 (SEQ ID NO:2381), CeresClone:569938 (SEQ ID NO:2383), GI:12323980 (SEQ ID NO:2384), CeresAnnot:1442362 (SEQ ID NO:2386), CeresClone:726598 (SEQ ID NO:2388), CeresClone:1814046 (SEQ ID NO:2390), GI:6554472 (SEQ ID NO:2391), GI:116780880 (SEQ ID NO:2392), CeresClone:221950 (SEQ ID NO:2394), CeresAnnot:8654926 (SEQ ID NO:2396), GI:116786601 (SEQ ID NO:2397), CeresAnnot:8654929 (SEQ ID NO:2399), CeresAnnot:8654924 (SEQ ID NO:2401), GI:3341511 (SEQ ID NO:2402), GI:104162062 (SEQ ID NO:2403), CeresClone:1895447 (SEQ ID NO:2405), CeresAnnot:8643098 (SEQ ID NO:2407), GI:25140432 (SEQ ID NO:2408), GI:65306612 (SEQ ID NO:2409), CeresClone:238838 (SEQ ID NO:2411), CeresAnnot:1490817 (SEQ ID NO:2413), CeresAnnot:1448084 (SEQ ID NO:2415), CeresAnnot:8713261 (SEQ ID NO:2417), GI:9964087 (SEQ ID NO:2418), GI:65306614 (SEQ ID NO:2419), CeresClone:1993306 (SEQ ID NO:2421), GI:3242328 (SEQ ID NO:2422), GI:9998901 (SEQ ID NO:2423), GI:194702744 (SEQ ID NO:2424), GI:2058311 (SEQ ID NO:2425), CeresAnnot:1490808 (SEQ ID NO:2427), GI:10304406 (SEQ ID NO:2428), GI:190710713 (SEQ ID NO:2429), GI:50345920 (SEQ ID NO:2430), CeresClone:2022068 (SEQ ID NO:2432), CeresAnnot:8743567 (SEQ ID NO:2434), or GI:212720841 (SEQ ID NO:2435). In some cases, a functional homolog of SEQ ID NO:2323 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2323

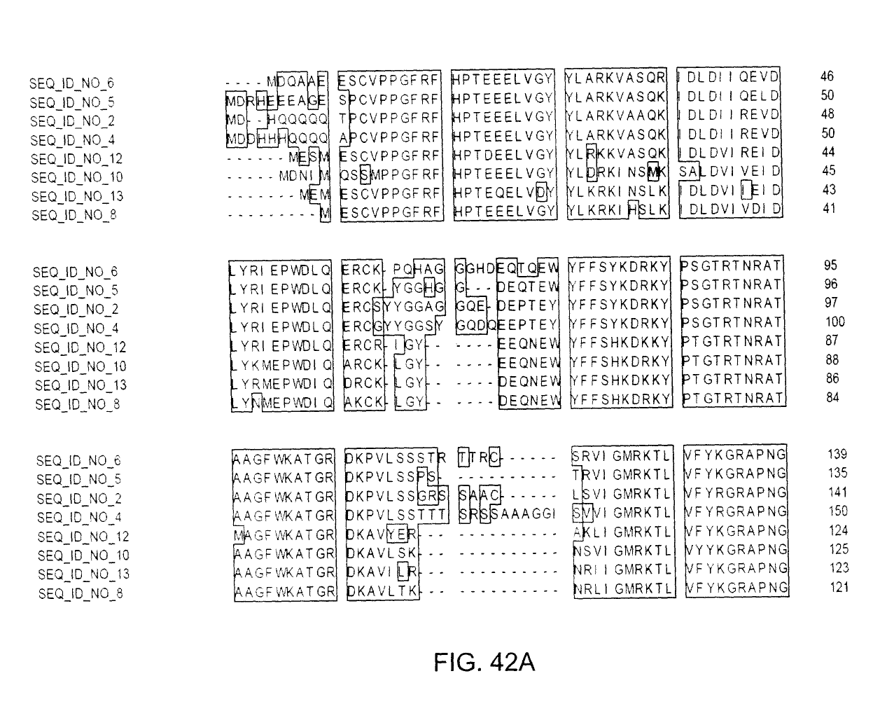

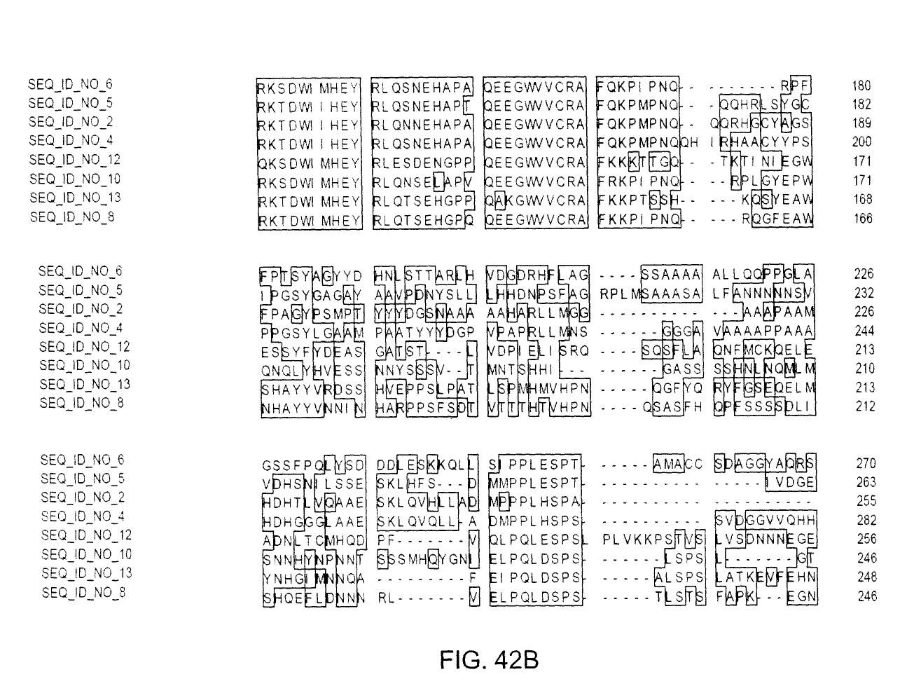

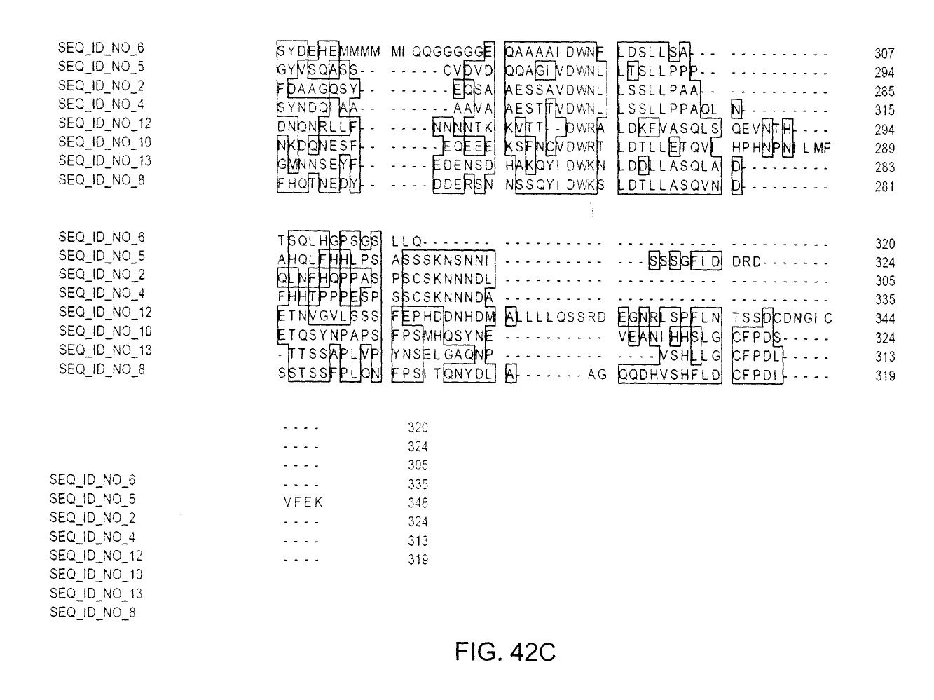

Examples of amino acid sequences of functional homologs of the polypeptide set forth in SEQ ID NO:2 are provided in FIG. 42 and in the Sequence Listing. Such functional homologs include, for example, CeresAnnot:8703827 (SEQ ID NO:4), GI:115474357 (SEQ ID NO:5), GI:212723110 (SEQ ID NO:6), CeresAnnot:1540152 (SEQ ID NO:8), CeresClone:151034 (SEQ ID NO:10), CeresClone:546186 (SEQ ID NO:12), GI:157352570 (SEQ ID NO:13), CeresClone:1707861 (SEQ ID NO:15), CeresAnnot:8703721 (SEQ ID NO:17), GI:125559843 (SEQ ID NO:18), CeresAnnot:1533215 (SEQ ID NO:20), CeresAnnot:8643427 (SEQ ID NO:22), CeresAnnot:8734475 (SEQ ID NO:24), GI:115483104 (SEQ ID NO:25), GI:115465900 (SEQ ID NO:26), GI:115450461 (SEQ ID NO:27), CeresClone:1579097 (SEQ ID NO:29), GI:38605889 (SEQ ID NO:30), CeresAnnot:849986 (SEQ ID NO:32), CeresAnnot:1486736 (SEQ ID NO:34), GI:125583042 (SEQ ID NO:35), CeresAnnot:827270 (SEQ ID NO:37), GI:157354896 (SEQ ID NO:38), GI:73759951 (SEQ ID NO:39), CeresClone:1848312 (SEQ ID NO:41), CeresAnnot:8702071 (SEQ ID NO:43), CeresAnnot:1692437 (SEQ ID NO:45), CeresAnnot:8681598 (SEQ ID NO:47), CeresAnnot:857774 (SEQ ID NO:49), CeresClone:1944387 (SEQ ID NO:51), CeresAnnot:832306 (SEQ ID NO:53), CeresClone:528201 (SEQ ID NO:55), GI:78499704 (SEQ ID NO:56), GI:194704142 (SEQ ID NO:57), CeresAnnot:836923 (SEQ ID NO:59), GI:115445339 (SEQ ID NO:60), CeresAnnot:8464728 (SEQ ID NO:62), GI:33321040 (SEQ ID NO:63), GI:157360723 (SEQ ID NO:64), GI:10086508 (SEQ ID NO:65), CeresClone:1877937 (SEQ ID NO:67), CeresAnnot:1533211 (SEQ ID NO:69), GI:124360182 (SEQ ID NO:70), GI:194700068 (SEQ ID NO:71), CeresAnnot:1487753 (SEQ ID NO:73), GI:32487489 (SEQ ID NO:74), GI:90399258 (SEQ ID NO:75), or CeresClone:630591 (SEQ ID NO:77). In some cases, a functional homolog of SEQ ID NO:2 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2.

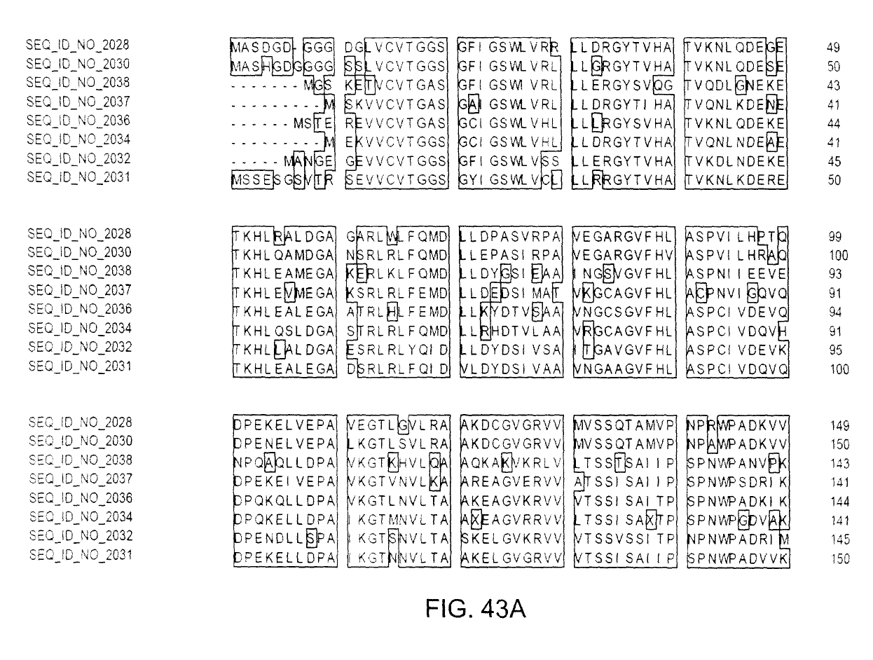

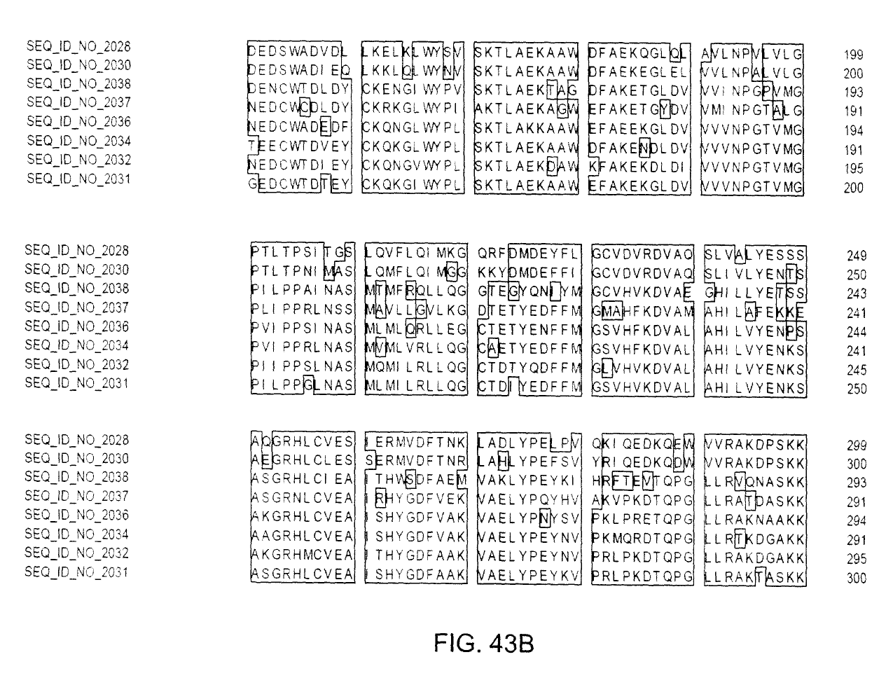

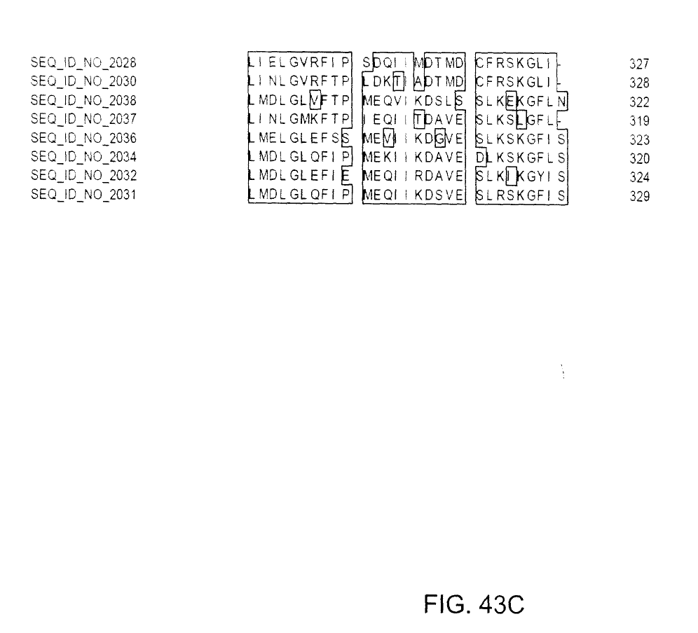

Examples of amino acid sequences of functional homologs of the polypeptide set forth in SEQ ID NO:2028 are provided in FIG. 43 and in the Sequence Listing. Such functional homologs include, for example, CeresAnnot:8669366 (SEQ ID NO:2030), GI:157337055 (SEQ ID NO:2031), GI:47900734 (SEQ ID NO:2032), CeresClone:626124 (SEQ ID NO:2034), CeresClone:1411238 (SEQ ID NO:2036), GI:68159360 (SEQ ID NO:2037), GI:116786528 (SEQ ID NO:2038), CeresAnnot:8669367 (SEQ ID NO:2040), GI:56202090 (SEQ ID NO:2041), GI:115440809 (SEQ ID NO:2042), GI:76573313 (SEQ ID NO:2043), GI:15237678 (SEQ ID NO:2044), CeresClone:30064 (SEQ ID NO:2046), GI:115456211 (SEQ ID NO:2047), GI:125588404 (SEQ ID NO:2048), GI:166080305 (SEQ ID NO:2049), CeresClone:931114 (SEQ ID NO:2051), CeresClone:257394 (SEQ ID NO:2053), CeresClone:339107 (SEQ ID NO:2055), GI:212275195 (SEQ ID NO:2056), CeresClone:1986400 (SEQ ID NO:2058), CeresAnnot:8631946 (SEQ ID NO:2060), CeresAnnot:8631947 (SEQ ID NO:2062), CeresClone:100845730 (SEQ ID NO:2064), CeresAnnot:1543142 (SEQ ID NO:2066), GI:15226955 (SEQ ID NO:2067), CeresClone:114042 (SEQ ID NO:2069), GI:157354338 (SEQ ID NO:2070), CeresClone:625502 (SEQ ID NO:2072), GI:2981475 (SEQ ID NO:2073), GI:17978649 (SEQ ID NO:2074), GI:115476626 (SEQ ID NO:2075), GI:170286897 (SEQ ID NO:2076), GI:162458118 (SEQ ID NO:2077), GI:28544965 (SEQ ID NO:2078), GI:28544959 (SEQ ID NO:2079), CeresClone:2023103 (SEQ ID NO:2081), CeresAnnot:1514866 (SEQ ID NO:2083), CeresClone:1724312 (SEQ ID NO:2085), CeresClone:1850477 (SEQ ID NO:2087), or GI:25140434 (SEQ ID NO:2088). In some cases, a functional homolog of SEQ ID NO:2028 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2028.

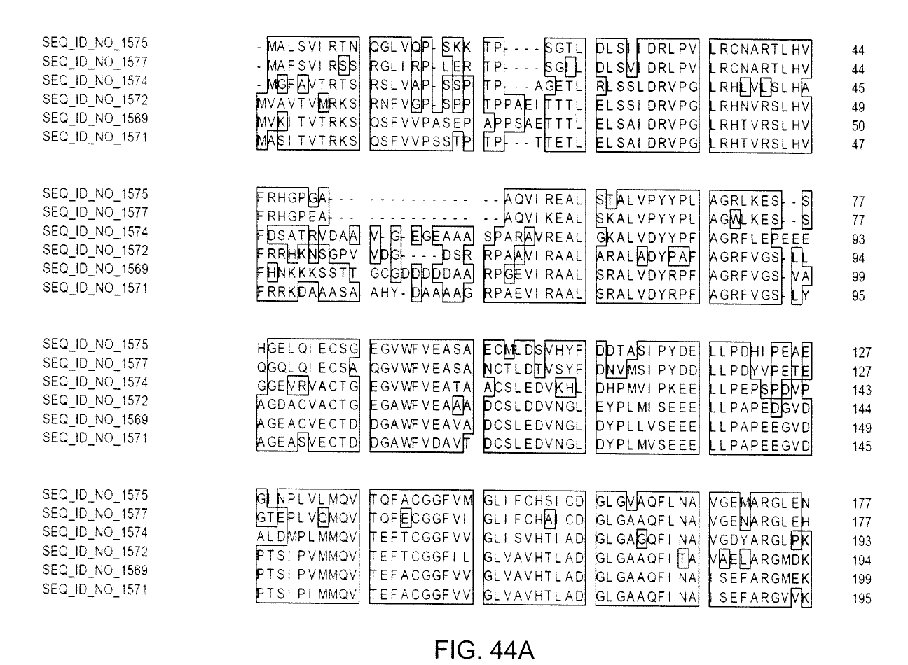

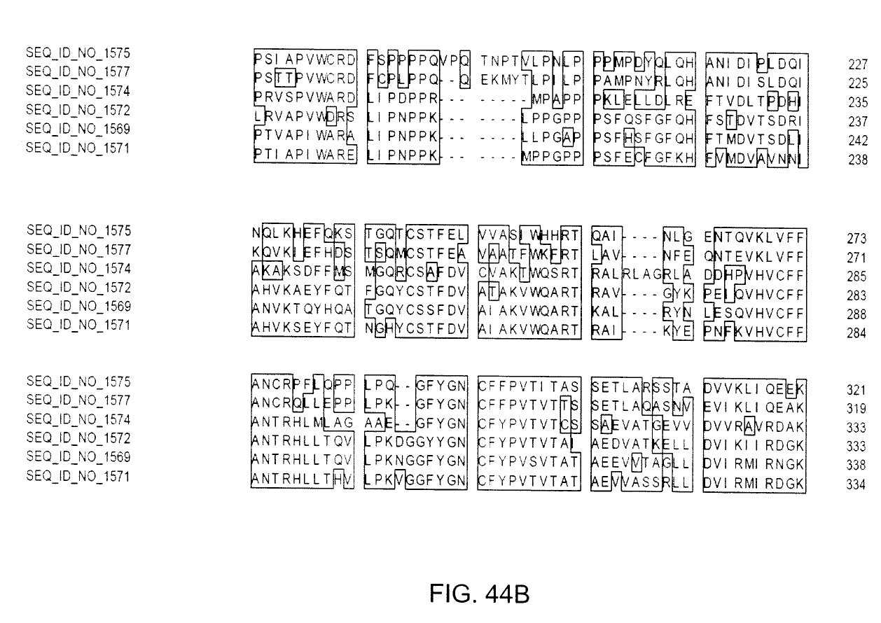

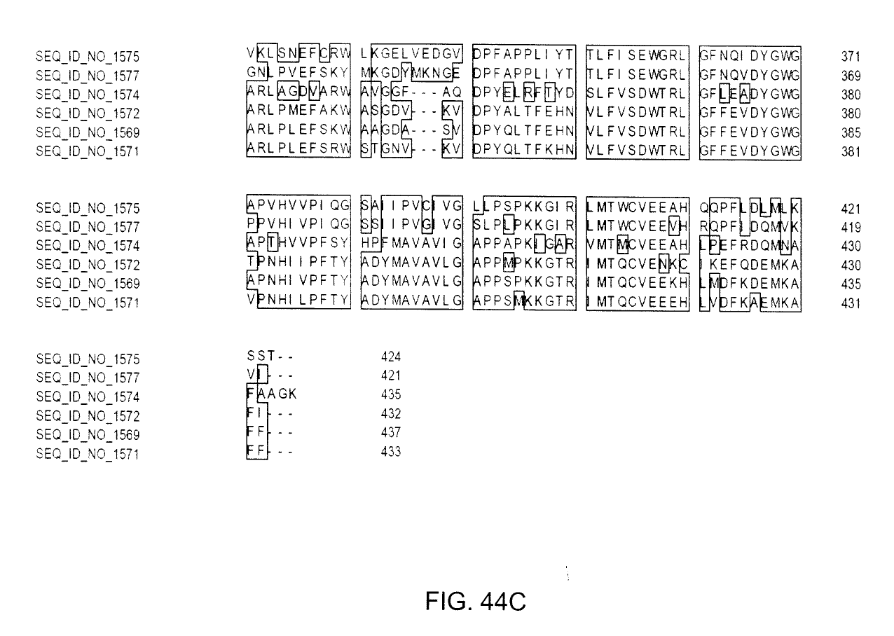

Examples of amino acid sequences of functional homologs of the polypeptide set forth in SEQ ID NO:1569 are provided in FIG. 44 and in the Sequence Listing. Such functional homologs include, for example, CeresClone:347043 (SEQ ID NO:1571), GI:115462965 (SEQ ID NO:1572), CeresClone:615256 (SEQ ID NO:1574), GI:225437789 (SEQ ID NO:1575), CeresAnnot:1471495 (SEQ ID NO:1577), GI:115436066 (SEQ ID NO:1578), CeresClone:1792947 (SEQ ID NO:1580), GI:115461963 (SEQ ID NO:1581), CeresClone:1804757 (SEQ ID NO:1583), GI:194707276 (SEQ ID NO:1584), CeresClone:1993287 (SEQ ID NO:1586), CeresClone:1791289 (SEQ ID NO:1588), GI:125597752 (SEQ ID NO:1589), CeresClone:346820 (SEQ ID NO:1591), CeresClone:1991829 (SEQ ID NO:1593), GI:125555936 (SEQ ID NO:1594), GI:115434876 (SEQ ID NO:1595), GI:115462409 (SEQ ID NO:1596), CeresClone:1790661 (SEQ ID NO:1598), GI:125551047 (SEQ ID NO:1599), GI:125524662 (SEQ ID NO:1600), CeresClone:1844794 (SEQ ID NO:1602), GI:151175361 (SEQ ID NO:1603), GI:151175359 (SEQ ID NO:1604), GI:150246875 (SEQ ID NO:1605), CeresClone:1811754 (SEQ ID NO:1607), CeresClone:334953 (SEQ ID NO:1609), CeresClone:1780550 (SEQ ID NO:1611), GI:115434962 (SEQ ID NO:1612), GI:125569315 (SEQ ID NO:1613), CeresClone:1973047 (SEQ ID NO:1615), GI:151175355 (SEQ ID NO:1616), CeresClone:283413 (SEQ ID NO:1618), GI:151175357 (SEQ ID NO:1619), CeresClone:1978685 (SEQ ID NO:1621), or GI:15228696 (SEQ ID NO:1622). In some cases, a functional homolog of SEQ ID NO:1569 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:1569.

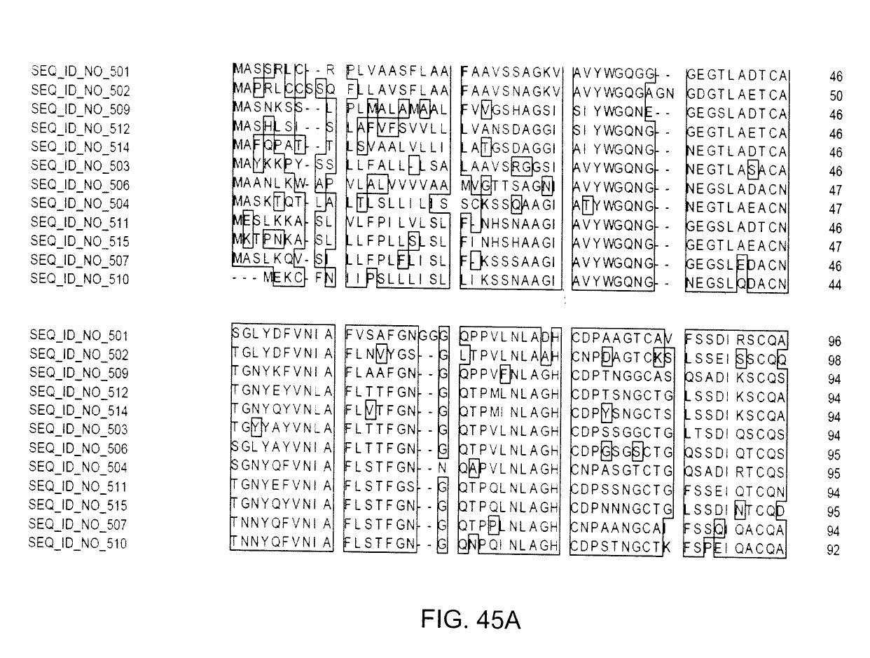

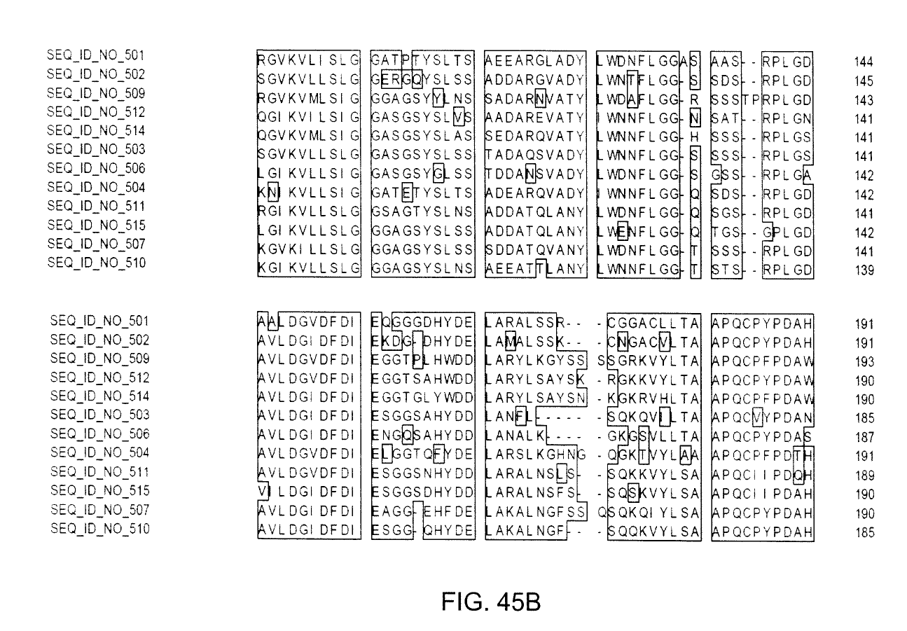

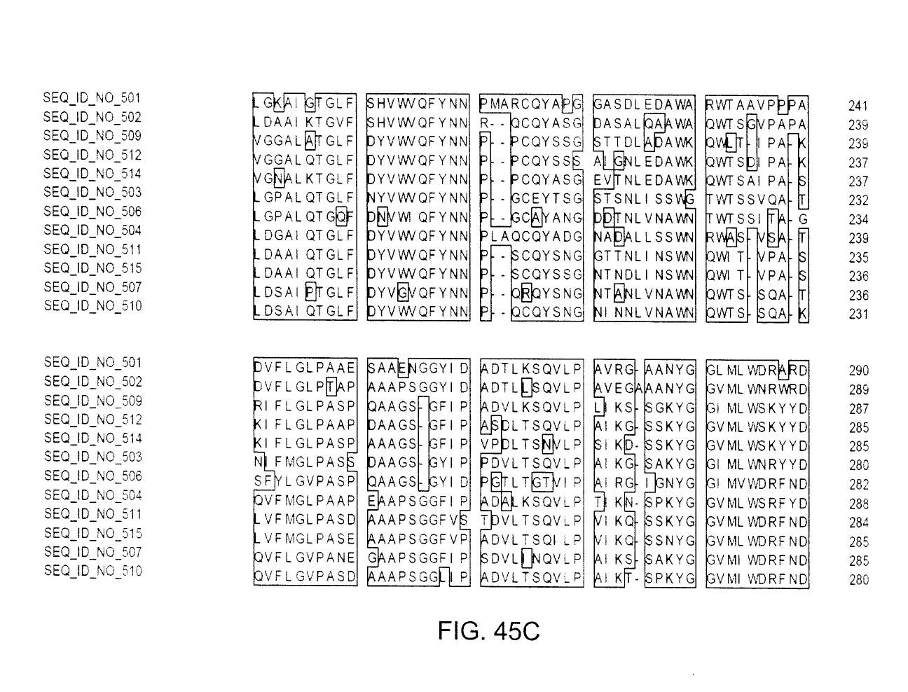

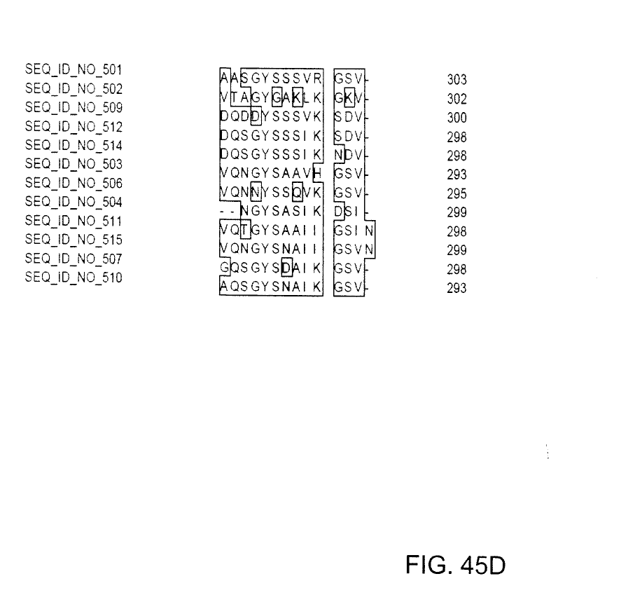

Examples of amino acid sequences of functional homologs of the polypeptide set forth in SEQ ID NO:501 are provided in FIG. 45 and in the Sequence Listing. Such functional homologs include, for example, GI:115439265 (SEQ ID NO:502), GI:189095136 (SEQ ID NO:503), GI:83853955 (SEQ ID NO:504), CeresClone:100882327 (SEQ ID NO:506), GI:13560118 (SEQ ID NO:507), CeresAnnot:8646122 (SEQ ID NO:509), GI:544006 (SEQ ID NO:510), GI:762879 (SEQ ID NO:511), GI:106647236 (SEQ ID NO:512), CeresAnnot:1471221 (SEQ ID NO:514), GI:4835582 (SEQ ID NO:515), GI:27817953 (SEQ ID NO:516), GI:10954033 (SEQ ID NO:517), GI:125557999 (SEQ ID NO:518), CeresAnnot:8646117 (SEQ ID NO:520), CeresAnnot:8646118 (SEQ ID NO:522), CeresAnnot:1467383 (SEQ ID NO:524), CeresAnnot:8646120 (SEQ ID NO:526), CeresAnnot:8667904 (SEQ ID NO:528), CeresAnnot:1467381 (SEQ ID NO:530), GI:115438985 (SEQ ID NO:531), GI:162462924 (SEQ ID NO:532), GI:33562677 (SEQ ID NO:533), GI:124294787 (SEQ ID NO:534), GI:116330 (SEQ ID NO:535), GI:4530607 (SEQ ID NO:536), GI:157349836 (SEQ ID NO:537), GI:75708015 (SEQ ID NO:538), GI:20975280 (SEQ ID NO:539), GI:157349835 (SEQ ID NO:540), CeresAnnot:1497300 (SEQ ID NO:542), GI:28971736 (SEQ ID NO:543), CeresClone:562682 (SEQ ID NO:545), GI:4835584 (SEQ ID NO:546), GI:157360148 (SEQ ID NO:547), GI:197253305 (SEQ ID NO:548), CeresAnnot:1483301 (SEQ ID NO:550), GI:147791187 (SEQ ID NO:551), CeresAnnot:1483300 (SEQ ID NO:553), GI:4689374 (SEQ ID NO:554), GI:157349837 (SEQ ID NO:555), CeresAnnot:1498645 (SEQ ID NO:557), CeresAnnot:1483298 (SEQ ID NO:559), GI:157349845 (SEQ ID NO:560), CeresAnnot:1471220 (SEQ ID NO:562), GI:3451147 (SEQ ID NO:563), CeresClone:536756 (SEQ ID NO:565), GI:15553476 (SEQ ID NO:566), CeresClone:630327 (SEQ ID NO:568), CeresAnnot:8669695 (SEQ ID NO:570), CeresAnnot:1471240 (SEQ ID NO:572), GI:20981684 (SEQ ID NO:573), CeresAnnot:1483302 (SEQ ID NO:575), GI:1705812 (SEQ ID NO:576), GI:119393870 (SEQ ID NO:577), CeresClone:631157 (SEQ ID NO:579), GI:33562679 (SEQ ID NO:580), GI:33323055 (SEQ ID NO:581), GI:125528455 (SEQ ID NO:582), GI:147805131 (SEQ ID NO:583), GI:147860736 (SEQ ID NO:584), GI:115441185 (SEQ ID NO:585), GI:10880383 (SEQ ID NO:586), GI:125572708 (SEQ ID NO:587), CeresAnnot:1467380 (SEQ ID NO:589), GI:2425170 (SEQ ID NO:590), GI:116328 (SEQ ID NO:591), GI:116332 (SEQ ID NO:592), GI:125570070 (SEQ ID NO:593), CeresAnnot:8643742 (SEQ ID NO:595), GI:11967922 (SEQ ID NO:596), GI:7595839 (SEQ ID NO:597), GI:2342459 (SEQ ID NO:598), GI:166664 (SEQ ID NO:599), CeresClone:1825429 (SEQ ID NO:601), GI:2342461 (SEQ ID NO:602), CeresAnnot:8669693 (SEQ ID NO:604), GI:2342453 (SEQ ID NO:605), CeresAnnot:8669692 (SEQ ID NO:607), CeresAnnot:570753 (SEQ ID NO:609), GI:2342443 (SEQ ID NO:610), GI:2342433 (SEQ ID NO:611), GI:22775364 (SEQ ID NO:612), GI:115441183 (SEQ ID NO:613), GI:22775368 (SEQ ID NO:614), GI:22775376 (SEQ ID NO:615), GI:22775370 (SEQ ID NO:616), GI:22775382 (SEQ ID NO:617), GI:22775358 (SEQ ID NO:618), GI:125528454 (SEQ ID NO:619), GI:2342439 (SEQ ID NO:620), GI:2342457 (SEQ ID NO:621), GI:22775380 (SEQ ID NO:622), GI:2342465 (SEQ ID NO:623), GI:2853142 (SEQ ID NO:624), GI:116337 (SEQ ID NO:625), GI:167540 (SEQ ID NO:626), or GI:28848952 (SEQ ID NO:627). In some cases, a functional homolog of SEQ ID NO:501 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:501.

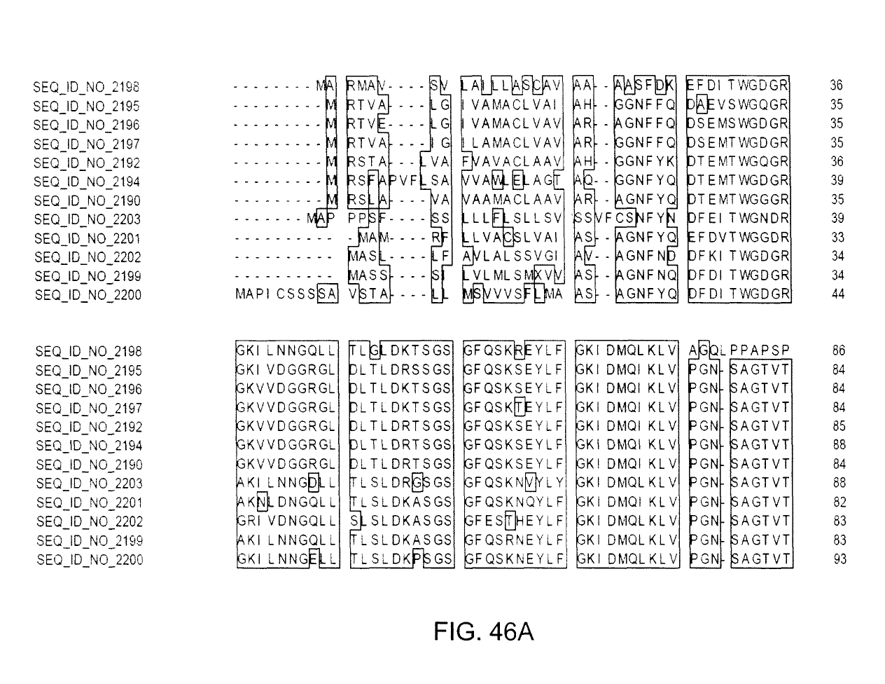

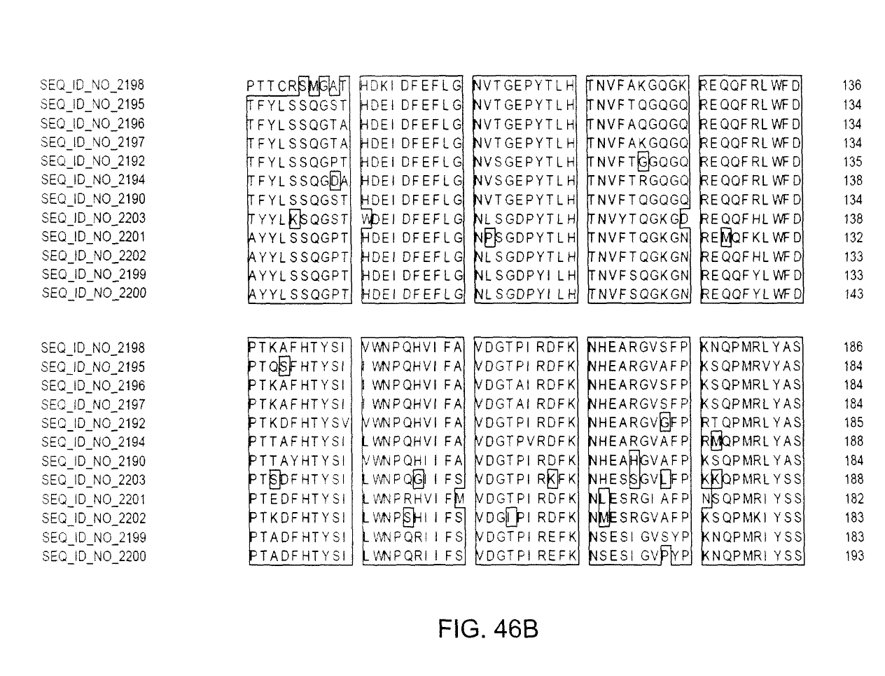

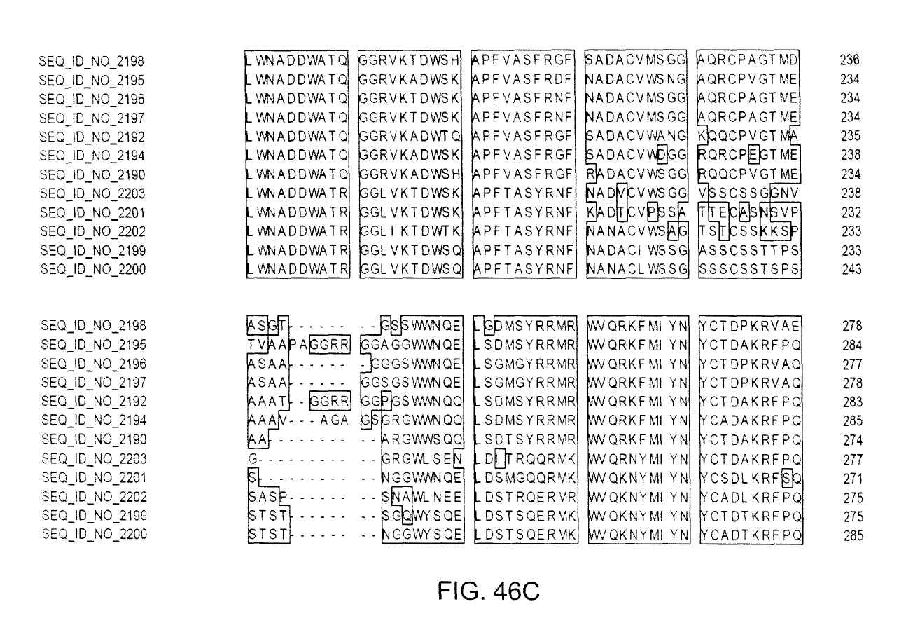

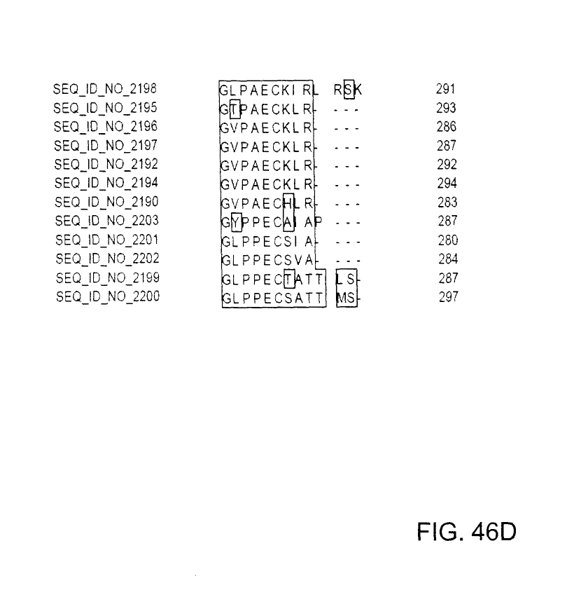

Examples of amino acid sequences of functional homologs of the polypeptide set forth in SEQ ID NO:2190 are provided in FIG. 46 and in the Sequence Listing. Such functional homologs include, for example, CeresAnnot:8744420 (SEQ ID NO:2192), CeresClone:331385 (SEQ ID NO:2194), GI:115469712 (SEQ ID NO:2195), GI:1890577 (SEQ ID NO:2196), GI:51039064 (SEQ ID NO:2197), GI:14330332 (SEQ ID NO:2198), GI:147854712 (SEQ ID NO:2199), GI:157352236 (SEQ ID NO:2200), GI:118722746 (SEQ ID NO:2201), GI:8886867 (SEQ ID NO:2202), GI:115334952 (SEQ ID NO:2203), CeresClone:1789502 (SEQ ID NO:2205), CeresClone:1805428 (SEQ ID NO:2207), CeresClone:1724099 (SEQ ID NO:2209), CeresClone:1724817 (SEQ ID NO:2211), CeresClone:1804995 (SEQ ID NO:2213), CeresClone:1446366 (SEQ ID NO:2215), CeresClone:1054422 (SEQ ID NO:2217), CeresClone:263803 (SEQ ID NO:2219), CeresClone:1821034 (SEQ ID NO:2221), CeresClone:1806021 (SEQ ID NO:2223), CeresClone:1727689 (SEQ ID NO:2225), GI:115469720 (SEQ ID NO:2226), CeresAnnot:8744425 (SEQ ID NO:2228), GI:212275237 (SEQ ID NO:2229), CeresClone:1724271 (SEQ ID NO:2231), CeresClone:247073 (SEQ ID NO:2233), CeresClone:1020658 (SEQ ID NO:2235), GI:1890575 (SEQ ID NO:2236), GI:225446111 (SEQ ID NO:2237), GI:225446115 (SEQ ID NO:2238), GI:147854714 (SEQ ID NO:2239), GI:68532877 (SEQ ID NO:2240), GI:147779866 (SEQ ID NO:2241), CeresClone:100062911 (SEQ ID NO:2243), GI:225446117 (SEQ ID NO:2244), CeresClone:1832719 (SEQ ID NO:2246), CeresClone:1793297 (SEQ ID NO:2248), CeresClone:1848637 (SEQ ID NO:2250), GI:225446103 (SEQ ID NO:2251), CeresAnnot:1362908 (SEQ ID NO:2253), CeresClone:100064069 (SEQ ID NO:2255), CeresAnnot:1469128 (SEQ ID NO:2257), CeresClone:656868 (SEQ ID NO:2259), CeresClone:1793334 (SEQ ID NO:2261), GI:29500891 (SEQ ID NO:2262), CeresClone:1895226 (SEQ ID NO:2264), GI:8886865 (SEQ ID NO:2265), CeresAnnot:878947 (SEQ ID NO:2267), CeresClone:1045431 (SEQ ID NO:2269), GI:22947852 (SEQ ID NO:2270), CeresClone:1855067 (SEQ ID NO:2272), GI:17064792 (SEQ ID NO:2273), CeresClone:662227 (SEQ ID NO:2275), GI:225446109 (SEQ ID NO:2276), CeresClone:522574 (SEQ ID NO:2278), GI:115334954 (SEQ ID NO:2279), CeresClone:581426 (SEQ ID NO:2281), GI:124109191 (SEQ ID NO:2282), CeresAnnot:1471882 (SEQ ID NO:2284), GI:34809190 (SEQ ID NO:2285), GI:29500893 (SEQ ID NO:2286), CeresAnnot:1452398 (SEQ ID NO:2288), GI:124109199 (SEQ ID NO:2289), CeresAnnot:1478206 (SEQ ID NO:2291), CeresAnnot:1445599 (SEQ ID NO:2293), CeresAnnot:1452397 (SEQ ID NO:2295), GI:19911573 (SEQ ID NO:2296), GI:124109181 (SEQ ID NO:2297), GI:22327914 (SEQ ID NO:2298), GI:42795468 (SEQ ID NO:2299), GI:42795462 (SEQ ID NO:2300), CeresAnnot:1466060 (SEQ ID NO:2302), CeresAnnot:8461207 (SEQ ID NO:2304), CeresAnnot:1506985 (SEQ ID NO:2306), GI:3901012 (SEQ ID NO:2307), CeresAnnot:1443040 (SEQ ID NO:2309), GI:90811697 (SEQ ID NO:2310), CeresAnnot:1443041 (SEQ ID NO:2312), GI:157358970 (SEQ ID NO:2313), GI:90656516 (SEQ ID NO:2314), GI:577066 (SEQ ID NO:2315), GI:90656520 (SEQ ID NO:2316), GI:88683124 (SEQ ID NO:2317), GI:90656518 (SEQ ID NO:2318), CeresAnnot:1482565 (SEQ ID NO:2320), or GI:15238891 (SEQ ID NO:2321). In some cases, a functional homolog of SEQ ID NO:2190 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2190.

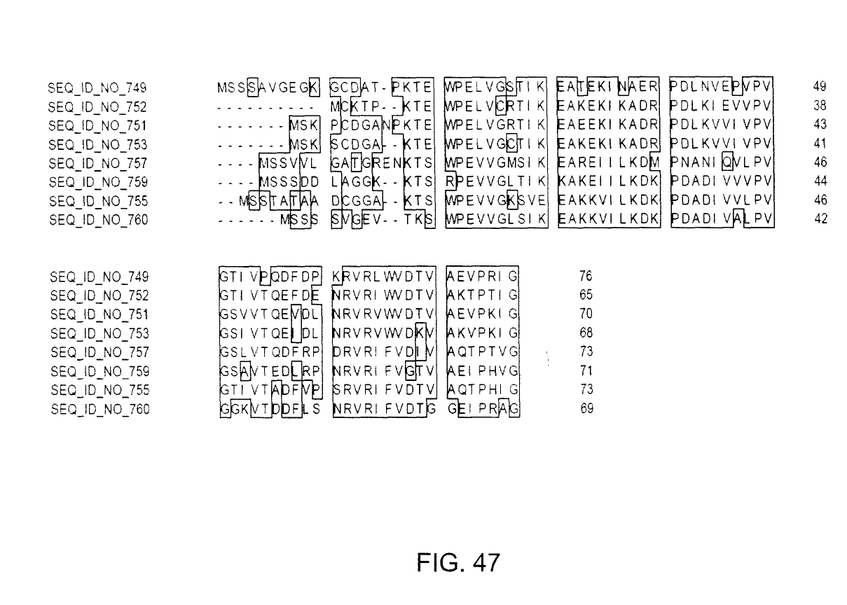

Examples of amino acid sequences of functional homologs of the polypeptide set forth in SEQ ID NO:749 are provided in FIG. 47 and in the Sequence Listing. Such functional homologs include, for example, CeresClone:1955766 (SEQ ID NO:749), CeresClone:570179 (SEQ ID NO:751), GI:54290293 (SEQ ID NO:752), GI:1617121 (SEQ ID NO:753), CeresClone:896724 (SEQ ID NO:757), CeresClone:607452 (SEQ ID NO:759), GI:37904392 (SEQ ID NO:760), CeresClone:1870473 (SEQ ID NO:762), CeresClone:2026564 (SEQ ID NO:764), CeresClone:2004365 (SEQ ID NO:766), CeresClone:2020677 (SEQ ID NO:768), CeresClone:2039538 (SEQ ID NO:770), CeresClone:844611 (SEQ ID NO:772), GI:125526847 (SEQ ID NO:773), CeresClone:597887 (SEQ ID NO:775), GI:58396949 (SEQ ID NO:776), CeresClone:684778 (SEQ ID NO:778), CeresClone:699511 (SEQ ID NO:780), CeresClone:1803377 (SEQ ID NO:782), CeresClone:1888961 (SEQ ID NO:784), CeresClone:897331 (SEQ ID NO:786), CeresClone:617775 (SEQ ID NO:788), GI:20513866 (SEQ ID NO:789), CeresAnnot:8724387 (SEQ ID NO:791), CeresClone:1804405 (SEQ ID NO:793), GI:48093396 (SEQ ID NO:794), GI:108862602 (SEQ ID NO:795), GI:115488400 (SEQ ID NO:796), CeresClone:759663 (SEQ ID NO:798), CeresClone:1801827 (SEQ ID NO:800), GI:48093418 (SEQ ID NO:801), GI:48093360 (SEQ ID NO:802), CeresClone:1457620 (SEQ ID NO:804), GI:48093370 (SEQ ID NO:805), CeresClone:639183 (SEQ ID NO:807), CeresClone:1453564 (SEQ ID NO:809), CeresClone:1531954 (SEQ ID NO:811), CeresClone:1460371 (SEQ ID NO:813), CeresClone:1627479 (SEQ ID NO:815), CeresClone:992630 (SEQ ID NO:817), CeresClone:685480 (SEQ ID NO:819), GI:75994159 (SEQ ID NO:820), CeresAnnot:8724380 (SEQ ID NO:822), GI:48093378 (SEQ ID NO:823), GI:75994143 (SEQ ID NO:824), GI:75994153 (SEQ ID NO:825), CeresAnnot:8724381 (SEQ ID NO:827), GI:75994157 (SEQ ID NO:828), CeresClone:730301 (SEQ ID NO:830), or CeresAnnot:8724388 (SEQ ID NO:832). In some cases, a functional homolog of SEQ ID NO:749 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:749.

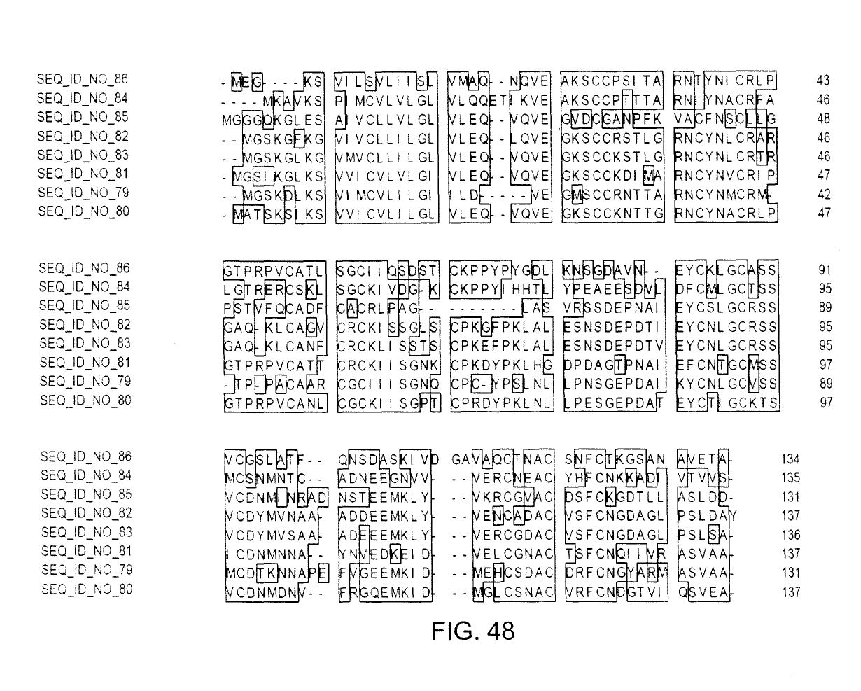

Examples of amino acid sequences of functional homologs of the polypeptide set forth in SEQ ID NO:79 are provided in FIG. 48 and in the Sequence Listing. Such functional homologs include, for example, GI:246215 (SEQ ID NO:80), GI:21069037 (SEQ ID NO:81), GI:4007850 (SEQ ID NO:82), GI:4007745 (SEQ ID NO:83), GI:54290571 (SEQ ID NO:84), GI:1052551 (SEQ ID NO:85), GI:802168 (SEQ ID NO:86), CeresClone:2047875 (SEQ ID NO:88), CeresClone:2024838 (SEQ ID NO:90), GI:246217 (SEQ ID NO:91), GI:1351242 (SEQ ID NO:92), GI:2507457 (SEQ ID NO:93), GI:44888362 (SEQ ID NO:94), GI:225958 (SEQ ID NO:95), GI:44888508 (SEQ ID NO:96), GI:401185 (SEQ ID NO:97), GI:21069039 (SEQ ID NO:98), GI:401184 (SEQ ID NO:99), GI:514332 (SEQ ID NO:100), GI:1304262 (SEQ ID NO:101), GI:21069045 (SEQ ID NO:102), GI:225008 (SEQ ID NO:103), GI:21069041 (SEQ ID NO:104), GI:2851607 (SEQ ID NO:105), GI:135788 (SEQ ID NO:106), GI:21069043 (SEQ ID NO:107), GI:4007846 (SEQ ID NO:108), GI:115468270 (SEQ ID NO:109), GI:125555528 (SEQ ID NO:110), GI:115468266 (SEQ ID NO:111), GI:54291433 (SEQ ID NO:112), or GI:1052819 (SEQ ID NO:113). In some cases, a functional homolog of SEQ ID NO:79 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:79.

Examples of amino acid sequences of functional homologs of the polypeptide set forth in SEQ ID NO:284 are provided in FIG. 49 and in the Sequence Listing. Such functional homologs include, for example, CeresAnnot:8679595 (SEQ ID NO:286), GI:115446685 (SEQ ID NO:287), GI:225447803 (SEQ ID NO:288), GI:30698042 (SEQ ID NO:289), CeresClone:611438 (SEQ ID NO:291), CeresClone:1953842 (SEQ ID NO:293), GI:38344065 (SEQ ID NO:294), GI:115458512 (SEQ ID NO:295), CeresAnnot:8700901 (SEQ ID NO:297), CeresAnnot:1446112 (SEQ ID NO:299), GI:157359819 (SEQ ID NO:300), CeresAnnot:1439208 (SEQ ID NO:302), CeresAnnot:1450728 (SEQ ID NO:304), CeresAnnot:1456235 (SEQ ID NO:306), GI:116310781 (SEQ ID NO:307), or GI:187940289 (SEQ ID NO:308). In some cases, a functional homolog of SEQ ID NO:284 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:284.

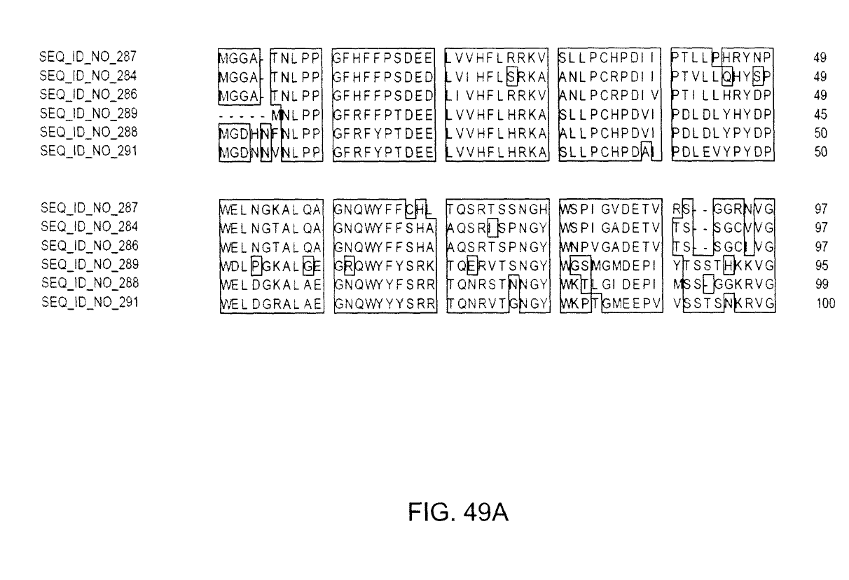

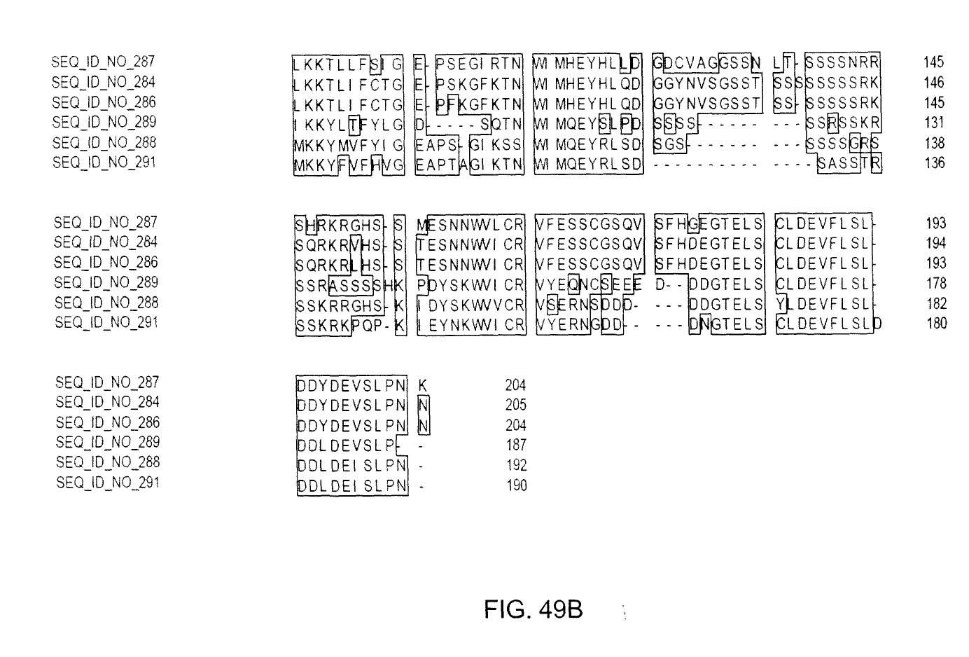

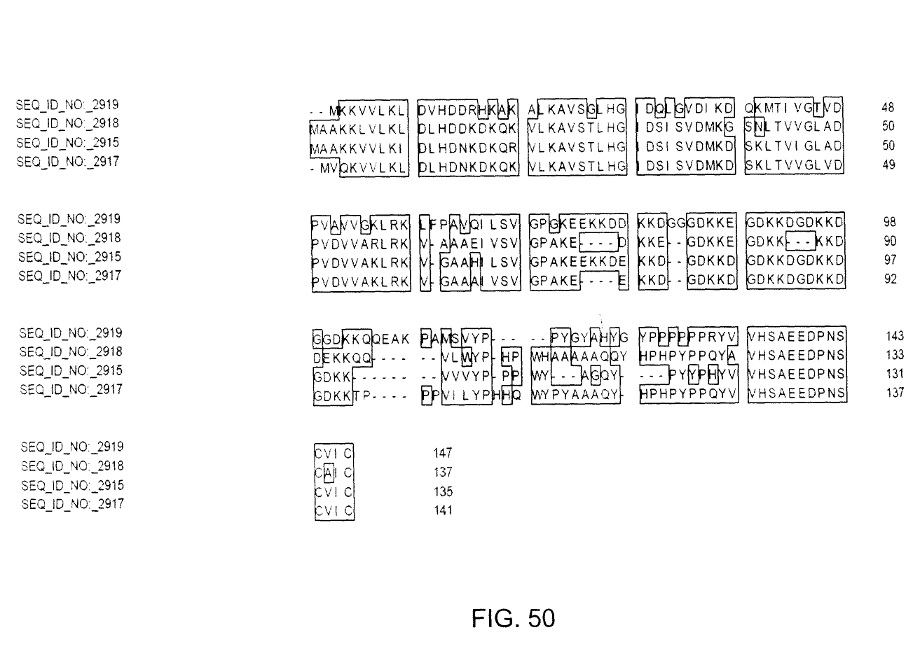

Examples of amino acid sequences of functional homologs for the polypeptide set forth in SEQ ID NO:2915 are provided in FIG. 50 and in the Sequence Listing. Such functional homologs include, for example, CeresAnnot:8703443 (SEQ ID NO:2917), GI:194702514 (SEQ ID NO:2918), CeresClone:699934 (SEQ ID NO:2919), CeresClone:1999925 (SEQ ID NO:2920), CeresClone:1830084 (SEQ ID NO:2921), CeresClone:1822001 (SEQ ID NO:2922), CeresClone:326580 (SEQ ID NO:2923), or GI:32488374 (SEQ ID NO:2924). In some cases, a functional homolog of SEQ ID NO:2915 has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2915.

The identification of conserved regions in a biomass-modulating polypeptide facilitates production of variants of biomass-modulating polypeptides. Variants of biomass-modulating polypeptides typically have 10 or fewer conservative amino acid substitutions within the primary amino acid sequence, e.g., 7 or fewer conservative amino acid substitutions, 5 or fewer conservative amino acid substitutions, or between 1 and 5 conservative substitutions. A useful variant polypeptide can be constructed based on one of the alignments set forth in FIG. 1, FIG. 2, FIG. 3, FIG. 4, FIG. 5, FIG. 6, FIG. 7, FIG. 8, FIG. 9, FIG. 10, FIG. 11, FIG. 12, FIG. 13, FIG. 14, FIG. 15, FIG. 16, FIG. 17, FIG. 18, FIG. 19, FIG. 20, FIG. 21, FIG. 22, FIG. 23, FIG. 24, FIG. 25, FIG. 26, FIG. 27, FIG. 28, FIG. 29, FIG. 30, FIG. 31, FIG. 32, FIG. 33, FIG. 34, FIG. 35, FIG. 36, FIG. 37, FIG. 38, FIG. 39, FIG. 40, FIG. 41, FIG. 42, FIG. 43, FIG. 44, FIG. 45, FIG. 46, FIG. 47, FIG. 48, FIG. 49, or FIG. 50 and/or homologs identified in the Sequence Listing. Such a polypeptide includes the conserved regions, arranged in the order depicted in the Figure from amino-terminal end to carboxy-terminal end. Such a polypeptide may also include zero, one, or more than one amino acid in positions marked by dashes. When no amino acids are present at positions marked by dashes, the length of such a polypeptide is the sum of the amino acid residues in all conserved regions. When amino acids are present at a position marked by dashes, such a polypeptide has a length that is the sum of the amino acid residues in all conserved regions and all dashes.

C. Functional Homologs Identified by HMMER

In some embodiments, useful biomass-modulating polypeptides include those that fit a Hidden Markov Model based on the polypeptides set forth in any one of FIGS. 1-50. A Hidden Markov Model (HMM) is a statistical model of a consensus sequence for a group of functional homologs. See, Durbin et al., Biological Sequence Analysis: Probabilistic Models of Proteins and Nucleic Acids, Cambridge University Press, Cambridge, UK (1998). An HMM is generated by the program HMMER 2.3.2 with default program parameters, using the sequences of the group of functional homologs as input. The multiple sequence alignment is generated by ProbCons (Do et al., Genome Res., 15(2):330-40 (2005)) version 1.11 using a set of default parameters: -c, —consistency REPS of 2; -ir, —iterative-refinement REPS of 100; -pre, —pre-training REPS of 0. ProbCons is a public domain software program provided by Stanford University.

The default parameters for building an HMM (hmmbuild) are as follows: the default “architecture prior” (archpri) used by MAP architecture construction is 0.85, and the default cutoff threshold (idlevel) used to determine the effective sequence number is 0.62. HMMER 2.3.2 was released Oct. 3, 2003 under a GNU general public license, and is available from various sources on the World Wide Web such as hmmer.janelia.org; hmmer wust1.edu; and fr.com/hmmer232/. Hmmbuild outputs the model as a text file.

The HMM for a group of functional homologs can be used to determine the likelihood that a candidate biomass-modulating polypeptide sequence is a better fit to that particular HMM than to a null HMM generated using a group of sequences that are not structurally or functionally related. The likelihood that a candidate polypeptide sequence is a better fit to an HMM than to a null HMM is indicated by the HMM bit score, a number generated when the candidate sequence is fitted to the HMM profile using the HMMER hmmsearch program. The following default parameters are used when running hmmsearch: the default E-value cutoff (E) is 10.0, the default bit score cutoff (T) is negative infinity, the default number of sequences in a database (Z) is the real number of sequences in the database, the default E-value cutoff for the per-domain ranked hit list (domE) is infinity, and the default bit score cutoff for the per-domain ranked hit list (domT) is negative infinity. A high HMM bit score indicates a greater likelihood that the candidate sequence carries out one or more of the biochemical or physiological function(s) of the polypeptides used to generate the HMM. A high HMM bit score is at least 20, and often is higher. Slight variations in the HMM bit score of a particular sequence can occur due to factors such as the order in which sequences are processed for alignment by multiple sequence alignment algorithms such as the ProbCons program. Nevertheless, such HMM bit score variation is minor.

The biomass-modulating polypeptides discussed below fit the indicated HMM with an HMM bit score greater than 93 (e.g., greater than 95, 96, 97, 98, 99, 100, 105, 110, 120, 130, 150, 170, 200, 300, 500, 1000, 1500, or 2000). In some embodiments, the HMM bit score of a biomass-modulating polypeptide discussed below is about 50%, 60%, 70%, 80%, 90%, or 95% of the HMM bit score of a functional homolog provided in the Sequence Listing of this application. In some embodiments, a biomass-modulating polypeptide discussed below fits the indicated HMM with an HMM bit score greater than 93, and has a domain indicative of a biomass-modulating polypeptide. In some embodiments, a biomass-modulating polypeptide discussed below fits the indicated HMM with an HMM bit score greater than 93, and has 65% or greater sequence identity (e.g., 75%, 80%, 85%, 90%, 95%, or 100% sequence identity) to an amino acid sequence shown in any one of FIGS. 1-50.

Examples of polypeptides are shown in the sequence listing that have HMM bit scores greater than 915 when fitted to an HMM generated from the amino acid sequences set forth in FIG. 1 and are identified in the Sequence Listing of this application. Such polypeptides include, for example, SEQ ID NOs: 2880, 2882, 2883, 2885, 2886, 2888, or 2890.

Examples of polypeptides are shown in the sequence listing that have HMM bit scores greater than 1149 when fitted to an HMM generated from the amino acid sequences set forth in FIG. 2 and are identified in the Sequence Listing of this application. Such polypeptides include, for example, SEQ ID NOs: 310, 311, 313, 315, 316, or 317.

Examples of polypeptides are shown in the sequence listing that have HMM bit scores greater than 618 when fitted to an HMM generated from the amino acid sequences set forth in FIG. 3 and are identified in the Sequence Listing of this application. Such polypeptides include, for example, SEQ ID NOs: 403, 404, 406, 408, 409, 410, 411, 412, or 414.

Examples of polypeptides are shown in the sequence listing that have HMM bit scores greater than 324 when fitted to an HMM generated from the amino acid sequences set forth in FIG. 4 and are identified in the Sequence Listing of this application. Such polypeptides include, for example, SEQ ID NOs: 669, 671, 672, 674, 676, or 677.

Examples of polypeptides are shown in the sequence listing that have HMM bit scores greater than 752 when fitted to an HMM generated from the amino acid sequences set forth in FIG. 5 and are identified in the Sequence Listing of this application. Such polypeptides include, for example, SEQ ID NOs: 1797, 1798, 1799, 1801, 1803, 1804, 1805, 1806, 1807, 1808, 1809, or 1810.

Examples of polypeptides are shown in the sequence listing that have HMM bit scores greater than 866 when fitted to an HMM generated from the amino acid sequences set forth in FIG. 6 and are identified in the Sequence Listing of this application. Such polypeptides include, for example, SEQ ID NOs: 717, 718, 719, 720, 721, or 722.

Examples of polypeptides are shown in the sequence listing that have HMM bit scores greater than 703 when fitted to an HMM generated from the amino acid sequences set forth in FIG. 7 and are identified in the Sequence Listing of this application. Such polypeptides include, for example, SEQ ID NOs: 693, 694, 696, 697, 698, or 700.

Examples of polypeptides are shown in the sequence listing that have HMM bit scores greater than 862 when fitted to an HMM generated from the amino acid sequences set forth in FIG. 8 and are identified in the Sequence Listing of this application. Such polypeptides include, for example, SEQ ID NOs: 1518, 1520, 1522, 1523, 1524, 1525, 1526, 1528, 1529, or 1531.

Examples of polypeptides are shown in the sequence listing that have HMM bit scores greater than 883 when fitted to an HMM generated from the amino acid sequences set forth in FIG. 9 and are identified in the Sequence Listing of this application. Such polypeptides include, for example, SEQ ID NOs: 1624, 1626, 1628, 1629, 1630, 1632, 1634, 1636, or 1637.

Examples of polypeptides are shown in the sequence listing that have HMM bit scores greater than 393 when fitted to an HMM generated from the amino acid sequences set forth in FIG. 10 and are identified in the Sequence Listing of this application. Such polypeptides include, for example, SEQ ID NOs: 1887, 1889, 1891, 1893, 1894, 1895, 1897, 1898, or 1900.

Examples of polypeptides are shown in the sequence listing that have HMM bit scores greater than 232 when fitted to an HMM generated from the amino acid sequences set forth in FIG. 11 and are identified in the Sequence Listing of this application. Such polypeptides include, for example, SEQ ID NOs: 834, 836, 838, 840, 842, 843, or 844.

Examples of polypeptides are shown in the sequence listing that have HMM bit scores greater than 815 when fitted to an HMM generated from the amino acid sequences set forth in FIG. 12 and are identified in the Sequence Listing of this application. Such polypeptides include, for example, SEQ ID NOs: 1318, 1320, 1322, 1324, 1325, 1326, 1328, 1329, 1330, 1332, 1333, 1334.

Examples of polypeptides are shown in the sequence listing that have HMM bit scores greater than 1251 when fitted to an HMM generated from the amino acid sequences set forth in FIG. 13 and are identified in the Sequence Listing of this application. Such polypeptides include, for example, SEQ ID NOs: 459, 461, 462, 464, 466, or 467.

Examples of polypeptides are shown in the sequence listing that have HMM bit scores greater than 1066 when fitted to an HMM generated from the amino acid sequences set forth in FIG. 14 and are identified in the Sequence Listing of this application. Such polypeptides include, for example, SEQ ID NOs: 335, 338, 342, 343, 358, 360, 377, 382, 389, 390, or 396.

Examples of polypeptides are shown in the sequence listing that have HMM bit scores greater than 1398 when fitted to an HMM generated from the amino acid sequences set forth in FIG. 15 and are identified in the Sequence Listing of this application. Such polypeptides include, for example, SEQ ID NOs: 846, 848, 849, 850, or 851.

Examples of polypeptides are shown in the sequence listing that have HMM bit scores greater than 370 when fitted to an HMM generated from the amino acid sequences set forth in FIG. 16 and are identified in the Sequence Listing of this application. Such polypeptides include, for example, SEQ ID NOs: 1426, 1428, 1429, 1431, 1433, 1435, 1437, 1439, 1440, 1441, 1443, 1445, 1446, or 1448.

Examples of polypeptides are shown in the sequence listing that have HMM bit scores greater than 328 when fitted to an HMM generated from the amino acid sequences set forth in FIG. 17 and are identified in the Sequence Listing of this application. Such polypeptides include, for example, SEQ ID NOs: 2525, 2527, 2529, 2530, 2531, or 2533.

Examples of polypeptides are shown in the sequence listing that have HMM bit scores greater than 133 when fitted to an HMM generated from the amino acid sequences set forth in FIG. 18 and are identified in the Sequence Listing of this application. Such polypeptides include, for example, SEQ ID NOs: 1272, 1274, 1276, 1278, 1279, 1281, 1283, or 1285.

Examples of polypeptides are shown in the sequence listing that have HMM bit scores greater than 265 when fitted to an HMM generated from the amino acid sequences set forth in FIG. 19 and are identified in the Sequence Listing of this application. Such polypeptides include, for example, SEQ ID NOs: 878, 880, 882, 883, 885, or 887.

Examples of polypeptides are shown in the sequence listing that have HMM bit scores greater than 843 when fitted to an HMM generated from the amino acid sequences set forth in FIG. 20 and are identified in the Sequence Listing of this application. Such polypeptides include, for example, SEQ ID NOs: 1660, 1661, 1662, 1664, 1666, or 1668.

Examples of polypeptides are shown in the sequence listing that have HMM bit scores greater than 93 when fitted to an HMM generated from the amino acid sequences set forth in FIG. 21 and are identified in the Sequence Listing of this application. Such polypeptides include, for example, SEQ ID NOs: 2654, 2656, 2658, 2659, 2661, 2663, 2665, 2667, 2668, 2670, 2672, 2674, 2675, 2677, or 2679.

Examples of polypeptides are shown in the sequence listing that have HMM bit scores greater than 175 when fitted to an HMM generated from the amino acid sequences set forth in FIG. 22 and are identified in the Sequence Listing of this application. Such polypeptides include, for example, SEQ ID NOs: 1137, 1139, 1141, 1142, 1144, or 1146.

Examples of polypeptides are shown in the sequence listing that have HMM bit scores greater than 2105 when fitted to an HMM generated from the amino acid sequences set forth in FIG. 23 and are identified in the Sequence Listing of this application. Such polypeptides include, for example, SEQ ID NOs: 2466, 2468, 2469, 2470, 2471, 2472, 2474, 2475, or 2476.

Examples of polypeptides are shown in the sequence listing that have HMM bit scores greater than 1213 when fitted to an HMM generated from the amino acid sequences set forth in FIG. 24 and are identified in the Sequence Listing of this application. Such polypeptides include, for example, SEQ ID NOs: 1107, 1110, 1111, 1112, 1113, 1114, 1115, or 1117.

Examples of polypeptides are shown in the sequence listing that have HMM bit scores greater than 1174 when fitted to an HMM generated from the amino acid sequences set forth in FIG. 25 and are identified in the Sequence Listing of this application. Such polypeptides include, for example, SEQ ID NOs: 1946, 1949, 1957, 1963, 1973, 1992, or 2022.

Examples of polypeptides are shown in the sequence listing that have HMM bit scores greater than 1138 when fitted to an HMM generated from the amino acid sequences set forth in FIG. 26 and are identified in the Sequence Listing of this application. Such polypeptides include, for example, SEQ ID NOs: 1054, 1056, 1058, 1060, 1061, 1062, 1064, 1066, or 1067.

Examples of polypeptides are shown in the sequence listing that have HMM bit scores greater than 1126 when fitted to an HMM generated from the amino acid sequences set forth in FIG. 27 and are identified in the Sequence Listing of this application. Such polypeptides include, for example, SEQ ID NOs: 115, 117, 119, 120, 121, 122, 123, 124, or 126.

Examples of polypeptides are shown in the sequence listing that have HMM bit scores greater than 1812 when fitted to an HMM generated from the amino acid sequences set forth in FIG. 28 and are identified in the Sequence Listing of this application. Such polypeptides include, for example, SEQ ID NOs: 2708, 2710, 2711, 2712, 2714, 2716, 2717, 2719, 2721, or 2722.

Examples of polypeptides are shown in the sequence listing that have HMM bit scores greater than 824 when fitted to an HMM generated from the amino acid sequences set forth in FIG. 29 and are identified in the Sequence Listing of this application. Such polypeptides include, for example, SEQ ID NOs: 1481, 1482, 1483, 1485, 1486, 1488, 1489, 1490, or 1492.

Examples of polypeptides are shown in the sequence listing that have HMM bit scores greater than 1240 when fitted to an HMM generated from the amino acid sequences set forth in FIG. 30 and are identified in the Sequence Listing of this application. Such polypeptides include, for example, SEQ ID NOs: 2748, 2750, 2751, 2753, 2754, 2756, 2758, or 2760.

Examples of polypeptides are shown in the sequence listing that have HMM bit scores greater than 1078 when fitted to an HMM generated from the amino acid sequences set forth in FIG. 31 and are identified in the Sequence Listing of this application. Such polypeptides include, for example, SEQ ID NOs: 947, 949, 950, 952, or 953.

Examples of polypeptides are shown in the sequence listing that have HMM bit scores greater than 873 when fitted to an HMM generated from the amino acid sequences set forth in FIG. 32 and are identified in the Sequence Listing of this application. Such polypeptides include, for example, SEQ ID NOs: 1727, 1729, 1731, 1732, or 1734.

Examples of polypeptides are shown in the sequence listing that have HMM bit scores greater than 1102 when fitted to an HMM generated from the amino acid sequences set forth in FIG. 33 and are identified in the Sequence Listing of this application. Such polypeptides include, for example, SEQ ID NOs: 2146, 2148, 2149, 2150, 2151, 2152, 2153, 2154, or 2156.

Examples of polypeptides are shown in the sequence listing that have HMM bit scores greater than 754 when fitted to an HMM generated from the amino acid sequences set forth in FIG. 34 and are identified in the Sequence Listing of this application. Such polypeptides include, for example, SEQ ID NOs: 2619, 2621, 2623, 2624, 2626, 2627, 2628, 2629, or 2631.

Examples of polypeptides are shown in the sequence listing that have HMM bit scores greater than 539 when fitted to an HMM generated from the amino acid sequences set forth in FIG. 35 and are identified in the Sequence Listing of this application. Such polypeptides include, for example, SEQ ID NOs: 211, 213, 214, 215, 217, 218, 219, or 220.

Examples of polypeptides are shown in the sequence listing that have HMM bit scores greater than 657 when fitted to an HMM generated from the amino acid sequences set forth in FIG. 36 and are identified in the Sequence Listing of this application. Such polypeptides include, for example, SEQ ID NOs: 629, 631, 633, 634, 635, 636, 637, or 638.

Examples of polypeptides are shown in the sequence listing that have HMM bit scores greater than 1924 when fitted to an HMM generated from the amino acid sequences set forth in FIG. 37 and are identified in the Sequence Listing of this application. Such polypeptides include, for example, SEQ ID NOs: 2544, 2546, 2547, 2548, 2550, 2551, or 2552.

Examples of polypeptides are shown in the sequence listing that have HMM bit scores greater than 1104 when fitted to an HMM generated from the amino acid sequences set forth in FIG. 38 and are identified in the Sequence Listing of this application. Such polypeptides include, for example, SEQ ID NOs: 2599, 2601, 2603, 2604, 2606, 2608, or 2609.

Examples of polypeptides are shown in the sequence listing that have HMM bit scores greater than 1347 when fitted to an HMM generated from the amino acid sequences set forth in FIG. 39 and are identified in the Sequence Listing of this application. Such polypeptides include, for example, SEQ ID NOs: 2437, 2439, 2440, 2441, 2442, 2443, 2445, 2446, or 2448.

Examples of polypeptides are shown in the sequence listing that have HMM bit scores greater than 1096 when fitted to an HMM generated from the amino acid sequences set forth in FIG. 40 and are identified in the Sequence Listing of this application. Such polypeptides include, for example, SEQ ID NOs: 2090, 2091, 2092, 2093, 2095, 2097, 2098, 2100, or 2102.

Examples of polypeptides are shown in the sequence listing that have HMM bit scores greater than 851 when fitted to an HMM generated from the amino acid sequences set forth in FIG. 41 and are identified in the Sequence Listing of this application. Such polypeptides include, for example, SEQ ID NOs: 2323, 2325, 2327, 2329, 2330, 2332, 2334, or 2335.

Examples of polypeptides are shown in the sequence listing that have HMM bit scores greater than 761 when fitted to an HMM generated from the amino acid sequences set forth in FIG. 42 and are identified in the Sequence Listing of this application. Such polypeptides include, for example, SEQ ID NOs: 2, 4, 5, 6, 8, 10, 12, or 13.

Examples of polypeptides are shown in the sequence listing that have HMM bit scores greater than 815 when fitted to an HMM generated from the amino acid sequences set forth in FIG. 43 and are identified in the Sequence Listing of this application. Such polypeptides include, for example, SEQ ID NOs: 2028, 2030, 2031, 2032, 2034, 2036, 2037, or 2038.

Examples of polypeptides are shown in the sequence listing that have HMM bit scores greater than 1121 when fitted to an HMM generated from the amino acid sequences set forth in FIG. 44 and are identified in the Sequence Listing of this application. Such polypeptides include, for example, SEQ ID NOs: 1569, 1571, 1572, 1574, 1575, or 1577.

Examples of polypeptides are shown in the sequence listing that have HMM bit scores greater than 681 when fitted to an HMM generated from the amino acid sequences set forth in FIG. 45 and are identified in the Sequence Listing of this application. Such polypeptides include, for example, SEQ ID NOs: 501, 502, 503, 504, 506, 507, 509, 510, 511, 512, 514, or 515.

Examples of polypeptides are shown in the sequence listing that have HMM bit scores greater than 677 when fitted to an HMM generated from the amino acid sequences set forth in FIG. 46 and are identified in the Sequence Listing of this application. Such polypeptides include, for example, SEQ ID NOs: 2190, 2192, 2194, 2195, 2196, 2197, 2198, 2199, 2200, 2201, 2202, or 2203.

Examples of polypeptides are shown in the sequence listing that have HMM bit scores greater than 146 when fitted to an HMM generated from the amino acid sequences set forth in FIG. 47 and are identified in the Sequence Listing of this application. Such polypeptides include, for example, SEQ ID NOs: 749, 751, 752, 753, 755, 757, 759, or 760.

Examples of polypeptides are shown in the sequence listing that have HMM bit scores greater than 286 when fitted to an HMM generated from the amino acid sequences set forth in FIG. 48 and are identified in the Sequence Listing of this application. Such polypeptides include, for example, SEQ ID NOs: 79, 80, 81, 82, 83, 84, 85, or 86.

Examples of polypeptides are shown in the sequence listing that have HMM bit scores greater than 493 when fitted to an HMM generated from the amino acid sequences set forth in FIG. 49 and are identified in the Sequence Listing of this application. Such polypeptides include, for example, SEQ ID NOs: 284, 286, 287, 288, 289, or 291.

Examples of polypeptides are shown in the sequence listing that have HMM bit scores greater than 316 when fitted to an HMM generated from the amino acid sequences set forth in FIG. 50 and are identified in the Sequence Listing of this application. Such polypeptides include, for example, SEQ ID NOs: 2915, 2917, 2918, or 2919.

D. Percent Identity

In some embodiments, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to one of the amino acid sequences set forth in SEQ ID NOs: 2, 4, 5, 6, 8, 10, 12, 13, 15, 17, 18, 20, 22, 24, 25, 26, 27, 29, 30, 32, 34, 35, 37, 38, 39, 41, 43, 45, 47, 49, 51, 53, 55, 56, 57, 59, 60, 62, 63, 64, 65, 67, 69, 70, 71, 73, 74, 75, 77, 79, 80, 81, 82, 83, 84, 85, 86, 88, 90, 91, 92, 93, 94, 95, 96, 97, 98, 99, 100, 101, 102, 100, 104, 105, 106, 107, 108, 109, 110, 111, 112, 113, 115, 117, 119, 120, 121, 122, 123, 124, 126, 128, 129, 131, 133, 135, 137, 139, 140, 141, 142, 144, 146, 147, 148, 149, 150, 152, 154, 155, 157, 158, 160, 162, 163, 165, 167, 169, 171, 173, 175, 176, 178, 180, 182, 183, 185, 187, 189, 191, 193, 194, 195, 196, 197, 198, 199, 200, 201, 203, 205, 207, 208, 209, 211, 213, 214, 215, 217, 218, 219, 220, 222, 223, 224, 226, 228, 229, 231, 232, 233, 234, 235, 236, 237, 238, 239, 240, 242, 244, 245, 246, 247, 248, 249, 250, 251, 252, 254, 255, 256, 257, 258, 259, 260, 261, 262, 263, 264, 265, 266, 267, 268, 269, 270, 271, 272, 273, 274, 275, 276, 277, 278, 279, 281, 282, 284, 286, 287, 288, 289, 291, 293, 294, 295, 297, 299, 300, 302, 304, 306, 307, 308, 310, 311, 313, 315, 316, 317, 318, 319, 320, 322, 324, 326, 328, 330, 331, 333, 335, 337, 338, 340, 342, 343, 345, 346, 347, 349, 350, 351, 353, 354, 355, 357, 358, 360, 362, 363, 364, 365, 367, 369, 370, 371, 372, 373, 374, 375, 377, 378, 379, 380, 382, 383, 384, 385, 386, 387, 389, 390, 391, 393, 395, 396, 398, 400, 401, 403, 404, 406, 408, 409, 410, 411, 412, 414, 415, 417, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 456, 457, 459, 461, 462, 464, 466, 467, 468, 469, 470, 472, 474, 475, 477, 479, 480, 482, 483, 484, 485, 487, 488, 489, 490, 491, 492, 493, 494, 495, 497, 499, 501, 502, 503, 504, 506, 507, 509, 510, 511, 512, 514, 515, 516, 517, 518, 520, 522, 524, 526, 528, 530, 531, 532, 533, 534, 535, 536, 537, 538, 539, 540, 542, 543, 545, 546, 547, 548, 550, 551, 553, 554, 555, 557, 559, 560, 562, 563, 565, 566, 568, 570, 572, 573, 575, 576, 577, 579, 580, 581, 582, 583, 584, 585, 586, 587, 589, 590, 591, 592, 593, 595, 596, 597, 598, 599, 601, 602, 604, 605, 607, 609, 610, 611, 612, 613, 614, 615, 616, 617, 618, 619, 620, 621, 622, 623, 624, 625, 626, 627, 629, 631, 633, 634, 635, 36, 637, 638, 640, 642, 644, 646, 648, 650, 651, 653, 654, 655, 656, 657, 658, 659, 660, 662, 663, 664, 665, 666, 667, 669, 671, 672, 674, 676, 677, 679, 680, 682, 684, 685, 687, 689, 690, 691, 693, 694, 696, 697, 698, 00, 702, 704, 705, 706, 707, 709, 710, 712, 714, 715, 717, 718, 719, 720, 721, 722, 723, 725, 726, 727, 728, 730, 731, 732, 733, 734, 735, 737, 739, 741, 743, 744, 745, 746, 747, 749, 751, 752, 753, 755, 757, 759, 760, 762, 764, 766, 768, 770, 772, 773, 775, 776, 778, 780, 782, 784, 786, 788, 789, 791, 793, 794, 795, 796, 798, 800, 801, 802, 804, 805, 807, 809, 811, 813, 815, 817, 819, 820, 822, 823, 824, 825, 827, 828, 830, 832, 834, 836, 838, 840, 842, 843, 844, 846, 848, 849, 850, 851, 853, 855, 856, 857, 858, 860, 861, 863, 864, 866, 868, 870, 871, 873, 874, 876, 878, 880, 882, 883, 885, 887, 889, 890, 892, 893, 895, 896, 897, 899, 901, 902, 903, 905, 907, 908, 909, 910, 912, 914, 915, 916, 917, 919, 921, 923, 925, 926, 928, 930, 932, 934, 935, 936, 937, 939, 941, 943, 945, 947, 949, 950, 952, 953, 955, 957, 959, 960, 962, 964, 965, 967, 968, 970, 972, 974, 976, 978, 980, 981, 983, 985, 86, 87, 989, 991, 993, 995, 997, 998, 1000, 1002, 1003, 1005, 1007, 1008, 1009, 1011, 1013, 1014, 1016, 1018, 1020, 1022, 1023, 1024, 1025, 1026, 1027, 1029, 1001, 1003, 1004, 1006, 1008, 1009, 1041, 1042, 1043, 1044, 1045, 1046, 1048, 1049, 1050, 1051, 1052, 1054, 1056, 1058, 1060, 1061, 1062, 1064, 1066, 1067, 1069, 1070, 1071, 1072, 1074, 1076, 1077, 1078, 1079, 1081, 1083, 1085, 1087, 1088, 1090, 1092, 1093, 1095, 1096, 1097, 1099, 1101, 1102, 1104, 1105, 1107, 1108, 1110, 1111, 1112, 1113, 1114, 1115, 1117, 1119, 1120, 1121, 1122, 1124, 1126, 1128, 1130, 1132, 1133, 1135, 1137, 1139, 1141, 1142, 1144, 1146, 1148, 1150, 1152, 1154, 1156, 1157, 1158, 1160, 1162, 1164, 1166, 1168, 1169, 1171, 1173, 1175, 1176, 1177, 1179, 1180, 1181, 1183, 1185, 1187, 1188, 1190, 1192, 1193, 1194, 1196, 1198, 1200, 1201, 1203, 1205, 1207, 1209, 1210, 1212, 1214, 1216, 1218, 1219, 1221, 1222, 1224, 1226, 1228, 1230, 1232, 1234, 1235, 1236, 1238, 1240, 1241, 1243, 1245, 1247, 1249, 1251, 1252, 1253, 1255, 1257, 1259, 1261, 1263, 1265, 1266, 1268, 1270, 1272, 1274, 1276, 1278, 1279, 1281, 1283, 1285, 1287, 1289, 1291, 1293, 1294, 1296, 1297, 1299, 1300, 1302, 1304, 1306, 1308, 1309, 1310, 1312, 1313, 1314, 1316, 1318, 1320, 1322, 1324, 1325, 1326, 1328, 1329, 1330, 1332, 1333, 1334, 1336, 1338, 1339, 1340, 1341, 1342, 1343, 1344, 1345, 1347, 1349, 1350, 1352, 1353, 1355, 1357, 1358, 1359, 1360, 1361, 1363, 1364, 1366, 1367, 1369, 1370, 1371, 1372, 1373, 1375, 1376, 1377, 1379, 1380, 1381, 1382, 1384, 1386, 1388, 1389, 1391, 1393, 1395, 1397, 1399, 1401, 1402, 1403, 1405, 1407, 1409, 1410, 1412, 1414, 1416, 1418, 1420, 1421, 1422, 1424, 1426, 1428, 1429, 1431, 1433, 1435, 1437, 1439, 1440, 1441, 1443, 1445, 1446, 1448, 1450, 1452, 1454, 1456, 1457, 1459, 1461, 1463, 1465, 1467, 1469, 1471, 1473, 1475, 1477, 1479, 1481, 1482, 1483, 1485, 1486, 1488, 1489, 1490, 1492, 1493, 1495, 1497, 1498, 1500, 1502, 1503, 1505, 1506, 1507, 1508, 1509, 1511, 1512, 1514, 1515, 1516, 1518, 1520, 1522, 1523, 1524, 1525, 1526, 1528, 1529, 1531, 1533, 1534, 1535, 1537, 1538, 1540, 1542, 1544, 1546, 1547, 1549, 1550, 1552, 1554, 1555, 1557, 1558, 1560, 1562, 1563, 1564, 1565, 1568, 1569, 1571, 1572, 1574, 1575, 1577, 1578, 1580, 1581, 1583, 1584, 1586, 1588, 1589, 1591, 1593, 1594, 1595, 1596, 1598, 1599, 1600, 1602, 1603, 1604, 1605, 1607, 1609, 1611, 1612, 1613, 1615, 1616, 1618, 1619, 1621, 1622, 1624, 1626, 1628, 1629, 1630, 1632, 1634, 1636, 1637, 1639, 1640, 1642, 1644, 1646, 1647, 1648, 1649, 1650, 1652, 1654, 1656, 1657, 1658, 1660, 1661, 1662, 1664, 1666, 1668, 1669, 1670, 1671, 1672, 1674, 1676, 1677, 1679, 1680, 1681, 1682, 1683, 1684, 1686, 1688, 1689, 1691, 1693, 1695, 1697, 1699, 1700, 1701, 1702, 1704, 1705, 1706, 1707, 1708, 1710, 1711, 1712, 1713, 1714, 1715, 1717, 1719, 1721, 1723, 1724, 1725, 1727, 1729, 1731, 1732, 1734, 1735, 1737, 1739, 1741, 1743, 1745, 1746, 1747, 1749, 1751, 1753, 1755, 1757, 1758, 1759, 1761, 1762, 1764, 1765, 1766, 1767, 1769, 1770, 1772, 1773, 1774, 1776, 1777, 1779, 1781, 1783, 1784, 1786, 1788, 1790, 1792, 1794, 1795, 1797, 798, 1799, 1801, 1803, 1804, 1805, 1806, 1807, 1808, 1809, 1810, 1811, 1812, 1813, 1815, 1816, 1817, 1819, 1821, 1822, 1823, 1825, 1826, 1827, 1828, 1829, 1831, 1833, 1834, 1835, 1837, 1839, 1841, 1842, 1844, 1845, 1847, 1849, 1850, 1852, 1853, 1854, 1855, 1856, 1857, 1858, 1859, 1861, 1863, 1865, 1866, 1867, 1869, 1871, 1872, 1874, 1876, 1877, 1879, 1881, 1882, 1883, 1885, 1887, 1889, 1891, 1893, 1894, 1895, 1897, 1898, 1900, 1902, 1903, 1904, 1906, 1908, 1910, 1912, 1913, 1915, 1917, 1918, 1919, 1920, 1922, 1924, 1926, 1928, 1929, 1931, 1933, 1935, 1936, 1938, 1940, 1942, 1944, 1946, 1948, 1949, 1951, 1953, 1955, 1957, 1959, 1961, 1963, 1965, 1967, 1969, 1971, 1972, 1973, 1974, 1975, 1976, 1978, 1979, 1980, 1982, 1983, 1985, 1986, 1987, 1989, 1990, 1992, 1994, 1996, 1997, 1999, 2000, 2002, 2003, 2005, 2007, 2008, 2009, 2010, 2011, 2013, 2015, 2016, 2017, 2018, 2019, 2021, 2022, 2023, 2024, 2026, 2028, 2030, 2031, 2032, 2034, 2036, 2037, 2038, 2040, 2041, 2042, 2043, 2044, 2046, 2047, 2048, 2049, 2051, 2053, 2055, 2056, 2058, 2060, 2062, 2064, 2066, 2067, 2069, 2070, 2072, 2073, 2074, 2075, 2076, 2077, 2078, 2079, 2081, 2083, 2085, 2087, 2088, 2090, 2091, 2092, 2093, 2095, 2097, 2098, 2100, 2102, 2104, 2105, 2106, 2108, 2110, 2112, 2113, 2115, 2117, 2118, 2119, 2120, 2121, 2122, 2124, 2126, 2128, 2129, 2130, 2132, 2133, 2135, 2136, 2137, 2138, 2140, 2141, 2142, 2143, 2144, 2146, 2148, 2149, 2150, 2151, 2152, 2153, 2154, 2156, 2158, 2159, 2160, 2161, 2162, 2163, 2165, 2167, 2169, 2170, 2171, 2172, 2173, 2174, 2175, 2176, 2177, 2178, 2179, 2181, 2183, 2184, 2186, 2187, 2188, 2190, 2192, 2194, 2195, 2196, 2197, 2198, 2199, 2200, 2201, 2202, 2203, 2205, 2207, 2209, 2211, 2213, 2215, 2217, 2219, 2221, 2223, 2225, 2226, 2228, 2229, 2231, 2233, 2235, 2236, 2237, 2238, 2239, 2240, 2241, 2243, 2244, 2246, 2248, 2250, 2251, 2253, 2255, 2257, 2259, 2261, 2262, 2264, 2265, 2267, 2269, 2270, 2272, 2273, 2275, 2276, 2278, 2279, 2281, 2282, 2284, 2285, 2286, 2288, 2289, 2291, 2293, 2295, 2296, 2297, 2298, 2299, 2300, 2302, 2304, 2306, 2307, 2309, 2310, 2312, 2313, 2314, 2315, 2316, 2317, 2318, 2320, 2321, 2323, 2325, 2327, 2329, 2330, 2332, 2334, 2335, 2337, 2339, 2341, 2343, 2345, 2346, 2347, 2348, 2350, 2352, 2354, 2356, 2357, 2358, 2360, 2361, 2362, 2364, 2366, 2367, 2369, 2371, 2373, 2375, 2376, 2378, 2380, 2381, 2383, 2384, 2386, 2388, 2390, 2391, 2392, 2394, 2396, 2397, 2399, 2401, 2402, 2403, 2405, 2407, 2408, 2409, 2411, 2413, 2415, 2417, 2418, 2419, 2421, 2422, 2423, 2424, 2425, 2427, 2428, 2429, 2430, 2432, 2434, 2435, 2437, 2439, 2440, 2441, 2442, 2443, 2445, 2446, 2448, 2449, 2451, 2452, 2454, 2456, 2457, 2458, 2459, 2460, 2461, 2462, 2463, 2464, 2466, 2468, 2469, 2470, 2471, 2472, 2474, 2475, 2476, 2477, 2478, 2480, 2482, 2483, 2484, 2485, 2486, 2487, 2488, 2489, 2490, 2491, 2493, 2494, 2495, 2496, 2497, 2498, 2499, 2500, 2502, 2503, 2505, 2506, 2507, 2509, 2510, 2511, 2512, 2513, 2514, 2516, 2517, 2518, 2519, 2521, 2522, 2523, 2525, 2527, 2529, 2530, 2531, 2533, 2535, 2537, 2539, 2540, 2541, 2542, 2544, 2546, 2547, 2548, 2550, 2551, 2552, 2554, 2556, 2557, 2558, 2559, 2561, 2562, 2564, 2565, 2567, 2569, 2571, 2572, 2573, 2574, 2575, 2576, 2578, 2580, 2581, 2583, 2584, 2585, 2587, 2588, 2589, 2590, 2592, 2593, 2594, 2595, 2597, 2599, 2601, 2603, 2604, 2606, 2608, 2609, 2611, 2612, 2614, 2615, 2616, 2617, 2619, 2621, 2623, 2624, 2626, 2627, 2628, 2629, 2631, 2632, 2634, 2636, 2638, 2640, 2641, 2642, 2644, 2646, 2647, 2648, 2649, 2650, 2652, 2654, 2656, 2658, 2659, 2661, 2663, 2665, 2667, 2668, 2670, 2672, 2674, 2675, 2677, 2679, 2681, 2683, 2685, 2687, 2689, 2691, 2693, 2695, 2697, 2699, 2701, 2703, 2704, 2706, 2708, 2710, 2711, 2712, 2714, 2716, 2717, 2719, 2721, 2722, 2724, 2726, 2728, 2730, 2732, 2734, 2735, 2736, 2738, 2739, 2740, 2741, 2743, 2745, 2746, 2748, 2750, 2751, 2753, 2754, 2756, 2758, 2760, 2762, 2764, 2766, 2768, 2770, 2771, 2772, 2773, 2775, 2777, 2778, 2780, 2781, 2782, 2784, 2786, 2788, 2790, 2791, 2792, 2794, 2796, 2797, 2799, 2801, 2802, 2803, 2804, 2806, 2807, 2809, 2810, 2811, 2813, 2815, 2816, 2818, 2819, 2821, 2822, 2823, 2825, 2826, 2828, 2830, 2832, 2833, 2835, 2836, 2838, 2840, 2842, 2844, 2845, 2846, 2847, 2849, 2850, 2851, 2852, 2853, 2855, 2856, 2857, 2858, 2859, 2861, 2862, 2863, 2864, 2866, 2867, 2868, 2869, 2870, 2872, 2873, 2875, 2876, 2877, 2878, 2880, 2882, 2883, 2885, 2886, 2888, 2890, 2891, 2892, 2893, 2894, 2896, 2898, 2900, 2902, 2903, 2904, 2906, 2907, 2908, 2909, 2910, 2911, 2915, 2917, 2918, 2919, 2920, 2921, 2922, 2923, 2924, or 2926. Polypeptides having such a percent sequence identity often have a domain indicative of a biomass-modulating polypeptide and/or have an HMM bit score that is greater than 93, as discussed above. Amino acid sequences of biomass-modulating polypeptides having at least 80% sequence identity to one of the amino acid sequences set forth in SEQ ID NOs: 2, 4, 5, 6, 8, 10, 12, 13, 15, 17, 18, 20, 22, 24, 25, 26, 27, 29, 30, 32, 34, 35, 37, 38, 39, 41, 43, 45, 47, 49, 51, 53, 55, 56, 57, 59, 60, 62, 63, 64, 65, 67, 69, 70, 71, 73, 74, 75, 77, 79, 80, 81, 82, 83, 84, 85, 86, 88, 90, 91, 92, 93, 94, 95, 96, 97, 98, 99, 100, 101, 102, 100, 104, 105, 106, 107, 108, 109, 110, 111, 112, 113, 115, 117, 119, 120, 121, 122, 123, 124, 126, 128, 129, 131, 133, 135, 137, 139, 140, 141, 142, 144, 146, 147, 148, 149, 150, 152, 154, 155, 157, 158, 160, 162, 163, 165, 167, 169, 171, 173, 175, 176, 178, 180, 182, 183, 185, 187, 189, 191, 193, 194, 195, 196, 197, 198, 199, 200, 201, 203, 205, 207, 208, 209, 211, 213, 214, 215, 217, 218, 219, 220, 222, 223, 224, 226, 228, 229, 231, 232, 233, 234, 235, 236, 237, 238, 239, 240, 242, 244, 245, 246, 247, 248, 249, 250, 251, 252, 254, 255, 256, 257, 258, 259, 260, 261, 262, 263, 264, 265, 266, 267, 268, 269, 270, 271, 272, 273, 274, 275, 276, 277, 278, 279, 281, 282, 284, 286, 287, 288, 289, 291, 293, 294, 295, 297, 299, 300, 302, 304, 306, 307, 308, 310, 311, 313, 315, 316, 317, 318, 319, 320, 322, 324, 326, 328, 330, 331, 333, 335, 337, 338, 340, 342, 343, 345, 346, 347, 349, 350, 351, 353, 354, 355, 357, 358, 360, 362, 363, 364, 365, 367, 369, 370, 371, 372, 373, 374, 375, 377, 378, 379, 380, 382, 383, 384, 385, 386, 387, 389, 390, 391, 393, 395, 396, 398, 400, 401, 403, 404, 406, 408, 409, 410, 411, 412, 414, 415, 417, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 456, 457, 459, 461, 462, 464, 466, 467, 468, 469, 470, 472, 474, 475, 477, 479, 480, 482, 483, 484, 485, 487, 488, 489, 490, 491, 492, 493, 494, 495, 497, 499, 501, 502, 503, 504, 506, 507, 509, 510, 511, 512, 514, 515, 516, 517, 518, 520, 522, 524, 526, 528, 530, 531, 532, 533, 534, 535, 536, 537, 538, 539, 540, 542, 543, 545, 546, 547, 548, 550, 551, 553, 554, 555, 557, 559, 560, 562, 563, 565, 566, 568, 570, 572, 573, 575, 576, 577, 579, 580, 581, 582, 583, 584, 585, 586, 587, 589, 590, 591, 592, 593, 595, 596, 597, 598, 599, 601, 602, 604, 605, 607, 609, 610, 611, 612, 613, 614, 615, 616, 617, 618, 619, 620, 621, 622, 623, 624, 625, 626, 627, 629, 631, 633, 634, 635, 36, 637, 638, 640, 642, 644, 646, 648, 650, 651, 653, 654, 655, 656, 657, 658, 659, 660, 662, 663, 664, 665, 666, 667, 669, 671, 672, 674, 676, 677, 679, 680, 682, 684, 685, 687, 689, 690, 691, 693, 694, 696, 697, 698, 00, 702, 704, 705, 706, 707, 709, 710, 712, 714, 715, 717, 718, 719, 720, 721, 722, 723, 725, 726, 727, 728, 730, 731, 732, 733, 734, 735, 737, 739, 741, 743, 744, 745, 746, 747, 749, 751, 752, 753, 755, 757, 759, 760, 762, 764, 766, 768, 770, 772, 773, 775, 776, 778, 780, 782, 784, 786, 788, 789, 791, 793, 794, 795, 796, 798, 800, 801, 802, 804, 805, 807, 809, 811, 813, 815, 817, 819, 820, 822, 823, 824, 825, 827, 828, 830, 832, 834, 836, 838, 840, 842, 843, 844, 846, 848, 849, 850, 851, 853, 855, 856, 857, 858, 860, 861, 863, 864, 866, 868, 870, 871, 873, 874, 876, 878, 880, 882, 883, 885, 887, 889, 890, 892, 893, 895, 896, 897, 899, 901, 902, 903, 905, 907, 908, 909, 910, 912, 914, 915, 916, 917, 919, 921, 923, 925, 926, 928, 930, 932, 934, 935, 936, 937, 939, 941, 943, 945, 947, 949, 950, 952, 953, 955, 957, 959, 960, 962, 964, 965, 967, 968, 970, 972, 974, 976, 978, 980, 981, 983, 985, 86, 87, 989, 991, 993, 995, 997, 998, 1000, 1002, 1003, 1005, 1007, 1008, 1009, 1011, 1013, 1014, 1016, 1018, 1020, 1022, 1023, 1024, 1025, 1026, 1027, 1029, 1001, 1003, 1004, 1006, 1008, 1009, 1041, 1042, 1043, 1044, 1045, 1046, 1048, 1049, 1050, 1051, 1052, 1054, 1056, 1058, 1060, 1061, 1062, 1064, 1066, 1067, 1069, 1070, 1071, 1072, 1074, 1076, 1077, 1078, 1079, 1081, 1083, 1085, 1087, 1088, 1090, 1092, 1093, 1095, 1096, 1097, 1099, 1101, 1102, 1104, 1105, 1107, 1108, 1110, 1111, 1112, 1113, 1114, 1115, 1117, 1119, 1120, 1121, 1122, 1124, 1126, 1128, 1130, 1132, 1133, 1135, 1137, 1139, 1141, 1142, 1144, 1146, 1148, 1150, 1152, 1154, 1156, 1157, 1158, 1160, 1162, 1164, 1166, 1168, 1169, 1171, 1173, 1175, 1176, 1177, 1179, 1180, 1181, 1183, 1185, 1187, 1188, 1190, 1192, 1193, 1194, 1196, 1198, 1200, 1201, 1203, 1205, 1207, 1209, 1210, 1212, 1214, 1216, 1218, 1219, 1221, 1222, 1224, 1226, 1228, 1230, 1232, 1234, 1235, 1236, 1238, 1240, 1241, 1243, 1245, 1247, 1249, 1251, 1252, 1253, 1255, 1257, 1259, 1261, 1263, 1265, 1266, 1268, 1270, 1272, 1274, 1276, 1278, 1279, 1281, 1283, 1285, 1287, 1289, 1291, 1293, 1294, 1296, 1297, 1299, 1300, 1302, 1304, 1306, 1308, 1309, 1310, 1312, 1313, 1314, 1316, 1318, 1320, 1322, 1324, 1325, 1326, 1328, 1329, 1330, 1332, 1333, 1334, 1336, 1338, 1339, 1340, 1341, 1342, 1343, 1344, 1345, 1347, 1349, 1350, 1352, 1353, 1355, 1357, 1358, 1359, 1360, 1361, 1363, 1364, 1366, 1367, 1369, 1370, 1371, 1372, 1373, 1375, 1376, 1377, 1379, 1380, 1381, 1382, 1384, 1386, 1388, 1389, 1391, 1393, 1395, 1397, 1399, 1401, 1402, 1403, 1405, 1407, 1409, 1410, 1412, 1414, 1416, 1418, 1420, 1421, 1422, 1424, 1426, 1428, 1429, 1431, 1433, 1435, 1437, 1439, 1440, 1441, 1443, 1445, 1446, 1448, 1450, 1452, 1454, 1456, 1457, 1459, 1461, 1463, 1465, 1467, 1469, 1471, 1473, 1475, 1477, 1479, 1481, 1482, 1483, 1485, 1486, 1488, 1489, 1490, 1492, 1493, 1495, 1497, 1498, 1500, 1502, 1503, 1505, 1506, 1507, 1508, 1509, 1511, 1512, 1514, 1515, 1516, 1518, 1520, 1522, 1523, 1524, 1525, 1526, 1528, 1529, 1531, 1533, 1534, 1535, 1537, 1538, 1540, 1542, 1544, 1546, 1547, 1549, 1550, 1552, 1554, 1555, 1557, 1558, 1560, 1562, 1563, 1564, 1565, 1568, 1569, 1571, 1572, 1574, 1575, 1577, 1578, 1580, 1581, 1583, 1584, 1586, 1588, 1589, 1591, 1593, 1594, 1595, 1596, 1598, 1599, 1600, 1602, 1603, 1604, 1605, 1607, 1609, 1611, 1612, 1613, 1615, 1616, 1618, 1619, 1621, 1622, 1624, 1626, 1628, 1629, 1630, 1632, 1634, 1636, 1637, 1639, 1640, 1642, 1644, 1646, 1647, 1648, 1649, 1650, 1652, 1654, 1656, 1657, 1658, 1660, 1661, 1662, 1664, 1666, 1668, 1669, 1670, 1671, 1672, 1674, 1676, 1677, 1679, 1680, 1681, 1682, 1683, 1684, 1686, 1688, 1689, 1691, 1693, 1695, 1697, 1699, 1700, 1701, 1702, 1704, 1705, 1706, 1707, 1708, 1710, 1711, 1712, 1713, 1714, 1715, 1717, 1719, 1721, 1723, 1724, 1725, 1727, 1729, 1731, 1732, 1734, 1735, 1737, 1739, 1741, 1743, 1745, 1746, 1747, 1749, 1751, 1753, 1755, 1757, 1758, 1759, 1761, 1762, 1764, 1765, 1766, 1767, 1769, 1770, 1772, 1773, 1774, 1776, 1777, 1779, 1781, 1783, 1784, 1786, 1788, 1790, 1792, 1794, 1795, 1797, 798, 1799, 1801, 1803, 1804, 1805, 1806, 1807, 1808, 1809, 1810, 1811, 1812, 1813, 1815, 1816, 1817, 1819, 1821, 1822, 1823, 1825, 1826, 1827, 1828, 1829, 1831, 1833, 1834, 1835, 1837, 1839, 1841, 1842, 1844, 1845, 1847, 1849, 1850, 1852, 1853, 1854, 1855, 1856, 1857, 1858, 1859, 1861, 1863, 1865, 1866, 1867, 1869, 1871, 1872, 1874, 1876, 1877, 1879, 1881, 1882, 1883, 1885, 1887, 1889, 1891, 1893, 1894, 1895, 1897, 1898, 1900, 1902, 1903, 1904, 1906, 1908, 1910, 1912, 1913, 1915, 1917, 1918, 1919, 1920, 1922, 1924, 1926, 1928, 1929, 1931, 1933, 1935, 1936, 1938, 1940, 1942, 1944, 1946, 1948, 1949, 1951, 1953, 1955, 1957, 1959, 1961, 1963, 1965, 1967, 1969, 1971, 1972, 1973, 1974, 1975, 1976, 1978, 1979, 1980, 1982, 1983, 1985, 1986, 1987, 1989, 1990, 1992, 1994, 1996, 1997, 1999, 2000, 2002, 2003, 2005, 2007, 2008, 2009, 2010, 2011, 2013, 2015, 2016, 2017, 2018, 2019, 2021, 2022, 2023, 2024, 2026, 2028, 2030, 2031, 2032, 2034, 2036, 2037, 2038, 2040, 2041, 2042, 2043, 2044, 2046, 2047, 2048, 2049, 2051, 2053, 2055, 2056, 2058, 2060, 2062, 2064, 2066, 2067, 2069, 2070, 2072, 2073, 2074, 2075, 2076, 2077, 2078, 2079, 2081, 2083, 2085, 2087, 2088, 2090, 2091, 2092, 2093, 2095, 2097, 2098, 2100, 2102, 2104, 2105, 2106, 2108, 2110, 2112, 2113, 2115, 2117, 2118, 2119, 2120, 2121, 2122, 2124, 2126, 2128, 2129, 2130, 2132, 2133, 2135, 2136, 2137, 2138, 2140, 2141, 2142, 2143, 2144, 2146, 2148, 2149, 2150, 2151, 2152, 2153, 2154, 2156, 2158, 2159, 2160, 2161, 2162, 2163, 2165, 2167, 2169, 2170, 2171, 2172, 2173, 2174, 2175, 2176, 2177, 2178, 2179, 2181, 2183, 2184, 2186, 2187, 2188, 2190, 2192, 2194, 2195, 2196, 2197, 2198, 2199, 2200, 2201, 2202, 2203, 2205, 2207, 2209, 2211, 2213, 2215, 2217, 2219, 2221, 2223, 2225, 2226, 2228, 2229, 2231, 2233, 2235, 2236, 2237, 2238, 2239, 2240, 2241, 2243, 2244, 2246, 2248, 2250, 2251, 2253, 2255, 2257, 2259, 2261, 2262, 2264, 2265, 2267, 2269, 2270, 2272, 2273, 2275, 2276, 2278, 2279, 2281, 2282, 2284, 2285, 2286, 2288, 2289, 2291, 2293, 2295, 2296, 2297, 2298, 2299, 2300, 2302, 2304, 2306, 2307, 2309, 2310, 2312, 2313, 2314, 2315, 2316, 2317, 2318, 2320, 2321, 2323, 2325, 2327, 2329, 2330, 2332, 2334, 2335, 2337, 2339, 2341, 2343, 2345, 2346, 2347, 2348, 2350, 2352, 2354, 2356, 2357, 2358, 2360, 2361, 2362, 2364, 2366, 2367, 2369, 2371, 2373, 2375, 2376, 2378, 2380, 2381, 2383, 2384, 2386, 2388, 2390, 2391, 2392, 2394, 2396, 2397, 2399, 2401, 2402, 2403, 2405, 2407, 2408, 2409, 2411, 2413, 2415, 2417, 2418, 2419, 2421, 2422, 2423, 2424, 2425, 2427, 2428, 2429, 2430, 2432, 2434, 2435, 2437, 2439, 2440, 2441, 2442, 2443, 2445, 2446, 2448, 2449, 2451, 2452, 2454, 2456, 2457, 2458, 2459, 2460, 2461, 2462, 2463, 2464, 2466, 2468, 2469, 2470, 2471, 2472, 2474, 2475, 2476, 2477, 2478, 2480, 2482, 2483, 2484, 2485, 2486, 2487, 2488, 2489, 2490, 2491, 2493, 2494, 2495, 2496, 2497, 2498, 2499, 2500, 2502, 2503, 2505, 2506, 2507, 2509, 2510, 2511, 2512, 2513, 2514, 2516, 2517, 2518, 2519, 2521, 2522, 2523, 2525, 2527, 2529, 2530, 2531, 2533, 2535, 2537, 2539, 2540, 2541, 2542, 2544, 2546, 2547, 2548, 2550, 2551, 2552, 2554, 2556, 2557, 2558, 2559, 2561, 2562, 2564, 2565, 2567, 2569, 2571, 2572, 2573, 2574, 2575, 2576, 2578, 2580, 2581, 2583, 2584, 2585, 2587, 2588, 2589, 2590, 2592, 2593, 2594, 2595, 2597, 2599, 2601, 2603, 2604, 2606, 2608, 2609, 2611, 2612, 2614, 2615, 2616, 2617, 2619, 2621, 2623, 2624, 2626, 2627, 2628, 2629, 2631, 2632, 2634, 2636, 2638, 2640, 2641, 2642, 2644, 2646, 2647, 2648, 2649, 2650, 2652, 2654, 2656, 2658, 2659, 2661, 2663, 2665, 2667, 2668, 2670, 2672, 2674, 2675, 2677, 2679, 2681, 2683, 2685, 2687, 2689, 2691, 2693, 2695, 2697, 2699, 2701, 2703, 2704, 2706, 2708, 2710, 2711, 2712, 2714, 2716, 2717, 2719, 2721, 2722, 2724, 2726, 2728, 2730, 2732, 2734, 2735, 2736, 2738, 2739, 2740, 2741, 2743, 2745, 2746, 2748, 2750, 2751, 2753, 2754, 2756, 2758, 2760, 2762, 2764, 2766, 2768, 2770, 2771, 2772, 2773, 2775, 2777, 2778, 2780, 2781, 2782, 2784, 2786, 2788, 2790, 2791, 2792, 2794, 2796, 2797, 2799, 2801, 2802, 2803, 2804, 2806, 2807, 2809, 2810, 2811, 2813, 2815, 2816, 2818, 2819, 2821, 2822, 2823, 2825, 2826, 2828, 2830, 2832, 2833, 2835, 2836, 2838, 2840, 2842, 2844, 2845, 2846, 2847, 2849, 2850, 2851, 2852, 2853, 2855, 2856, 2857, 2858, 2859, 2861, 2862, 2863, 2864, 2866, 2867, 2868, 2869, 2870, 2872, 2873, 2875, 2876, 2877, 2878, 2880, 2882, 2883, 2885, 2886, 2888, 2890, 2891, 2892, 2893, 2894, 2896, 2898, 2900, 2902, 2903, 2904, 2906, 2907, 2908, 2909, 2910, 2911, 2915, 2917, 2918, 2919, 2920, 2921, 2922, 2923, 2924, or 2926 are provided in FIGS. 1-50 and in the Sequence Listing.

“Percent sequence identity” refers to the degree of sequence identity between any given reference sequence, e.g., SEQ ID NO: 1, and a candidate biomass-modulating sequence. A candidate sequence typically has a length that is from 80 percent to 200 percent of the length of the reference sequence, e.g., 82, 85, 87, 89, 90, 93, 95, 97, 99, 100, 105, 110, 115, 120, 130, 140, 150, 160, 170, 180, 190, or 200 percent of the length of the reference sequence. A percent identity for any candidate nucleic acid or polypeptide relative to a reference nucleic acid or polypeptide can be determined as follows. A reference sequence (e.g., a nucleic acid sequence or an amino acid sequence) is aligned to one or more candidate sequences using the computer program ClustalW (version 1.83, default parameters), which allows alignments of nucleic acid or polypeptide sequences to be carried out across their entire length (global alignment). Chenna et al., Nucleic Acids Res., 31(13):3497-500 (2003).

ClustalW calculates the best match between a reference and one or more candidate sequences, and aligns them so that identities, similarities and differences can be determined. Gaps of one or more residues can be inserted into a reference sequence, a candidate sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The ClustalW output is a sequence alignment that reflects the relationship between sequences. ClustalW can be run, for example, at the Baylor College of Medicine Search Launcher site on the World Wide Web (searchlauncher.bcm.tmc.edu/multi-align/multi-align.html) and at the European Bioinformatics Institute site on the World Wide Web (ebi.ac.uk/clustalw).

To determine percent identity of a candidate nucleic acid or amino acid sequence to a reference sequence, the sequences are aligned using ClustalW, the number of identical matches in the alignment is divided by the length of the reference sequence, and the result is multiplied by 100. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2880 Amino acid sequences of polypeptides having greater than 45% sequence identity to the polypeptide set forth in SEQ ID NO:2880 are provided in FIG. 1 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:310 Amino acid sequences of polypeptides having greater than 45% sequence identity to the polypeptide set forth in SEQ ID NO:310 are provided in FIG. 2 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:403 Amino acid sequences of polypeptides having greater than 45% sequence identity to the polypeptide set forth in SEQ ID NO:403 are provided in FIG. 3 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:669 Amino acid sequences of polypeptides having greater than 45% sequence identity to the polypeptide set forth in SEQ ID NO:669 are provided in FIG. 4 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:1797 Amino acid sequences of polypeptides having greater than 45% sequence identity to the polypeptide set forth in SEQ ID NO:1797 are provided in FIG. 5 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:717 Amino acid sequences of polypeptides having greater than 45% sequence identity to the polypeptide set forth in SEQ ID NO:717 are provided in FIG. 6 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:693 Amino acid sequences of polypeptides having greater than 45% sequence identity to the polypeptide set forth in SEQ ID NO:693 are provided in FIG. 7 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:1518 Amino acid sequences of polypeptides having greater than 45% sequence identity to the polypeptide set forth in SEQ ID NO:1518 are provided in FIG. 8 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:1624 Amino acid sequences of polypeptides having greater than 45% sequence identity to the polypeptide set forth in SEQ ID NO:1624 are provided in FIG. 9 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:1887 Amino acid sequences of polypeptides having greater than 45% sequence identity to the polypeptide set forth in SEQ ID NO:1887 are provided in FIG. 10 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:834 Amino acid sequences of polypeptides having greater than 45% sequence identity to the polypeptide set forth in SEQ ID NO:834 are provided in FIG. 11 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:1318 Amino acid sequences of polypeptides having greater than 45% sequence identity to the polypeptide set forth in SEQ ID NO:1318 are provided in FIG. 12 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:459 Amino acid sequences of polypeptides having greater than 45% sequence identity to the polypeptide set forth in SEQ ID NO:459 are provided in FIG. 13 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:335 Amino acid sequences of polypeptides having greater than 45% sequence identity to the polypeptide set forth in SEQ ID NO:335 are provided in FIG. 14 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:846 Amino acid sequences of polypeptides having greater than 45% sequence identity to the polypeptide set forth in SEQ ID NO:846 are provided in FIG. 15 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:1426 Amino acid sequences of polypeptides having greater than 45% sequence identity to the polypeptide set forth in SEQ ID NO: 1426 are provided in FIG. 16 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2525 Amino acid sequences of polypeptides having greater than 45% sequence identity to the polypeptide set forth in SEQ ID NO:2525 are provided in FIG. 17 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:1272 Amino acid sequences of polypeptides having greater than 45% sequence identity to the polypeptide set forth in SEQ ID NO:1272 are provided in FIG. 18 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:878 Amino acid sequences of polypeptides having greater than 45% sequence identity to the polypeptide set forth in SEQ ID NO:878 are provided in FIG. 19 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:1660 Amino acid sequences of polypeptides having greater than 45% sequence identity to the polypeptide set forth in SEQ ID NO:1660 are provided in FIG. 20 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2654 Amino acid sequences of polypeptides having greater than 45% sequence identity to the polypeptide set forth in SEQ ID NO:2654 are provided in FIG. 21 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:1137 Amino acid sequences of polypeptides having greater than 45% sequence identity to the polypeptide set forth in SEQ ID NO:1137 are provided in FIG. 22 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2466 Amino acid sequences of polypeptides having greater than 45% sequence identity to the polypeptide set forth in SEQ ID NO:2466 are provided in FIG. 23 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:1107 Amino acid sequences of polypeptides having greater than 45% sequence identity to the polypeptide set forth in SEQ ID NO:1107 are provided in FIG. 24 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:1946 Amino acid sequences of polypeptides having greater than 45% sequence identity to the polypeptide set forth in SEQ ID NO:1946 are provided in FIG. 25 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:1054 Amino acid sequences of polypeptides having greater than 45% sequence identity to the polypeptide set forth in SEQ ID NO:1054 are provided in FIG. 26 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:115 Amino acid sequences of polypeptides having greater than 45% sequence identity to the polypeptide set forth in SEQ ID NO:115 are provided in FIG. 27 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2708 Amino acid sequences of polypeptides having greater than 45% sequence identity to the polypeptide set forth in SEQ ID NO:2708 are provided in FIG. 28 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:1481 Amino acid sequences of polypeptides having greater than 45% sequence identity to the polypeptide set forth in SEQ ID NO:1481 are provided in FIG. 29 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2748 Amino acid sequences of polypeptides having greater than 45% sequence identity to the polypeptide set forth in SEQ ID NO:2748 are provided in FIG. 30 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:947 Amino acid sequences of polypeptides having greater than 45% sequence identity to the polypeptide set forth in SEQ ID NO:947 are provided in FIG. 31 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:1727 Amino acid sequences of polypeptides having greater than 45% sequence identity to the polypeptide set forth in SEQ ID NO:1727 are provided in FIG. 32 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2146 Amino acid sequences of polypeptides having greater than 45% sequence identity to the polypeptide set forth in SEQ ID NO:2146 are provided in FIG. 33 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2619 Amino acid sequences of polypeptides having greater than 45% sequence identity to the polypeptide set forth in SEQ ID NO:2619 are provided in FIG. 34 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:211 Amino acid sequences of polypeptides having greater than 45% sequence identity to the polypeptide set forth in SEQ ID NO:211 are provided in FIG. 35 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:629 Amino acid sequences of polypeptides having greater than 45% sequence identity to the polypeptide set forth in SEQ ID NO:629 are provided in FIG. 36 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2544 Amino acid sequences of polypeptides having greater than 45% sequence identity to the polypeptide set forth in SEQ ID NO:2544 are provided in FIG. 37 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2599 Amino acid sequences of polypeptides having greater than 45% sequence identity to the polypeptide set forth in SEQ ID NO:2599 are provided in FIG. 38 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2437 Amino acid sequences of polypeptides having greater than 45% sequence identity to the polypeptide set forth in SEQ ID NO:2437 are provided in FIG. 39 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2090 Amino acid sequences of polypeptides having greater than 45% sequence identity to the polypeptide set forth in SEQ ID NO:2090 are provided in FIG. 40 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2323 Amino acid sequences of polypeptides having greater than 45% sequence identity to the polypeptide set forth in SEQ ID NO:2323 are provided in FIG. 41 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2. Amino acid sequences of polypeptides having greater than 45% sequence identity to the polypeptide set forth in SEQ ID NO:2 are provided in FIG. 42 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2028 Amino acid sequences of polypeptides having greater than 45% sequence identity to the polypeptide set forth in SEQ ID NO:2028 are provided in FIG. 43 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:1569 Amino acid sequences of polypeptides having greater than 45% sequence identity to the polypeptide set forth in SEQ ID NO:1569 are provided in FIG. 44 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:501 Amino acid sequences of polypeptides having greater than 45% sequence identity to the polypeptide set forth in SEQ ID NO:501 are provided in FIG. 45 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:2190 Amino acid sequences of polypeptides having greater than 45% sequence identity to the polypeptide set forth in SEQ ID NO:2190 are provided in FIG. 46 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:749 Amino acid sequences of polypeptides having greater than 45% sequence identity to the polypeptide set forth in SEQ ID NO:749 are provided in FIG. 47 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:79 Amino acid sequences of polypeptides having greater than 45% sequence identity to the polypeptide set forth in SEQ ID NO:79 are provided in FIG. 48 and in the Sequence Listing.

In some cases, a biomass-modulating polypeptide has an amino acid sequence with at least 45% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:284 Amino acid sequences of polypeptides having greater than 45% sequence identity to the polypeptide set forth in SEQ ID NO:284 are provided in FIG. 49 and in the Sequence Listing.

E. Other Sequences

It should be appreciated that a biomass-modulating polypeptide can include additional amino acids that are not involved in biomass modulation, and thus such a polypeptide can be longer than would otherwise be the case. For example, a biomass-modulating polypeptide can include a purification tag, a chloroplast transit peptide, a mitochondrial transit peptide, an amyloplast peptide, or a leader sequence added to the amino or carboxy terminus In some embodiments, a biomass-modulating polypeptide includes an amino acid sequence that functions as a reporter, e.g., a green fluorescent protein or yellow fluorescent protein.

### III. Nucleic Acids

Nucleic acids described herein include nucleic acids that are effective to modulate biomass levels when transcribed in a plant or plant cell. Such nucleic acids include, without limitation, those that encode a biomass-modulating polypeptide and those that can be used to inhibit expression of a biomass-modulating polypeptide via a nucleic acid based method.

A. Nucleic Acids Encoding Biomass-Modulating Polypeptides

Nucleic acids encoding biomass-modulating polypeptides are described herein. Examples of such nucleic acids include SEQ ID NOs: 1, 3, 7, 9, 11, 14, 16, 19, 21, 23, 28, 31, 33, 36, 40, 42, 44, 46, 48, 50, 52, 54, 58, 61, 66, 68, 72, 76, 78, 87, 89, 114, 116, 118, 125, 127, 130, 132, 134, 136, 138, 143, 145, 151, 153, 156, 159, 161, 164, 166, 168, 170, 172, 174, 177, 179, 181, 184, 186, 188, 190, 192, 202, 204, 206, 210, 212, 216, 221, 225, 227, 230, 241, 243, 253, 280, 283, 285, 290, 292, 296, 298, 301, 303, 305, 309, 312, 314, 321, 323, 325, 327, 329, 332, 334, 336, 339, 341, 344, 348, 352, 356, 359, 361, 366, 368, 376, 381, 388, 392, 394, 397, 399, 402, 405, 407, 413, 416, 418, 455, 458, 460, 463, 465, 471, 473, 476, 478, 481, 486, 496, 498, 500, 505, 508, 513, 519, 521, 523, 525, 527, 529, 541, 544, 549, 552, 556, 558, 561, 564, 567, 569, 571, 574, 578, 588, 594, 600, 603, 606, 608, 628, 630, 632, 639, 641, 643, 645, 647, 649, 652, 661, 668, 670, 673, 675, 678, 681, 683, 686, 688, 692, 695, 699, 701, 703, 708, 711, 713, 716, 724, 729, 736, 738, 740, 742, 748, 750, 754, 756, 758, 761, 763, 765, 767, 769, 771, 774, 777, 779, 781, 783, 785, 787, 790, 792, 797, 799, 803, 806, 808, 810, 812, 814, 816, 818, 821, 826, 829, 831, 833, 835, 837, 839, 841, 845, 847, 852, 854, 859, 862, 865, 867, 869, 872, 875, 877, 879, 881, 884, 886, 888, 891, 894, 898, 900, 904, 906, 911, 913, 918, 920, 922, 924, 927, 929, 931, 933, 938, 940, 942, 944, 946, 948, 951, 954, 956, 958, 961, 963, 966, 969, 971, 973, 975, 977, 979, 982, 984, 988, 990, 992, 994, 996, 999, 1001, 1004, 1006, 1010, 1012, 1015, 1017, 1019, 1021, 1028, 1000, 1002, 1005, 1007, 1040, 1047, 1053, 1055, 1057, 1059, 1063, 1065, 1068, 1073, 1075, 1080, 1082, 1084, 1086, 1089, 1091, 1094, 1098, 1100, 1100, 1106, 1109, 1116, 1118, 1123, 1125, 1127, 1129, 1131, 1134, 1136, 1138, 1140, 1143, 1145, 1147, 1149, 1151, 1153, 1155, 1159, 1161, 1163, 1165, 1167, 1170, 1172, 1174, 1178, 1182, 1184, 1186, 1189, 1191, 1195, 1197, 1199, 1202, 1204, 1206, 1208, 1211, 1213, 1215, 1217, 1220, 1223, 1225, 1227, 1229, 1231, 1233, 1237, 1239, 1242, 1244, 1246, 1248, 1250, 1254, 1256, 1258, 1260, 1262, 1264, 1267, 1269, 1271, 1273, 1275, 1277, 1280, 1282, 1284, 1286, 1288, 1290, 1292, 1295, 1298, 1301, 1303, 1305, 1307, 1311, 1315, 1317, 1319, 1321, 1323, 1327, 1331, 1335, 1337, 1346, 1348, 1351, 1354, 1356, 1362, 1365, 1368, 1374, 1378, 1383, 1385, 1387, 1390, 1392, 1394, 1396, 1398, 1400, 1404, 1406, 1408, 1411, 1413, 1415, 1417, 1419, 1423, 1425, 1427, 1430, 1432, 1434, 1436, 1438, 1442, 1444, 1447, 1449, 1451, 1453, 1455, 1458, 1460, 1462, 1464, 1466, 1468, 1470, 1472, 1474, 1476, 1478, 1480, 1484, 1487, 1491, 1494, 1496, 1499, 1501, 1504, 1510, 1513, 1517, 1519, 1521, 1527, 1530, 1532, 1536, 1539, 1541, 1543, 1545, 1548, 1551, 1553, 1556, 1559, 1561, 1566, 1568, 1570, 1573, 1576, 1579, 1582, 1585, 1587, 1590, 1592, 1597, 1601, 1606, 1608, 1610, 1614, 1617, 1620, 1623, 1625, 1627, 1631, 1633, 1635, 1638, 1641, 1643, 1645, 1651, 1653, 1655, 1659, 1663, 1665, 1667, 1673, 1675, 1678, 1685, 1687, 1690, 1692, 1694, 1696, 1698, 1703, 1709, 1716, 1718, 1720, 1722, 1726, 1728, 1730, 1733, 1736, 1738, 1740, 1742, 1744, 1748, 1750, 1752, 1754, 1756, 1760, 1763, 1768, 1771, 1775, 1778, 1780, 1782, 1785, 1787, 1789, 1791, 1793, 1796, 1800, 1802, 1814, 1818, 1820, 1824, 1830, 1832, 1836, 1838, 1840, 1843, 1846, 1848, 1851, 1860, 1862, 1864, 1868, 1870, 1873, 1875, 1878, 1880, 1884, 1886, 1888, 1890, 1892, 1896, 1899, 1901, 1905, 1907, 1909, 1911, 1914, 1916, 1921, 1923, 1925, 1927, 1930, 1932, 1934, 1937, 1939, 1941, 1943, 1945, 1947, 1950, 1952, 1954, 1956, 1958, 1960, 1962, 1964, 1966, 1968, 1970, 1977, 1981, 1984, 1988, 1991, 1993, 1995, 1998, 2001, 2004, 2006, 2012, 2014, 2020, 2025, 2027, 2029, 2033, 2035, 2039, 2045, 2050, 2052, 2054, 2057, 2059, 2061, 2063, 2065, 2068, 2071, 2080, 2082, 2084, 2086, 2089, 2094, 2096, 2099, 2101, 2100, 2107, 2109, 2111, 2114, 2116, 2123, 2125, 2127, 2131, 2134, 2139, 2145, 2147, 2155, 2157, 2164, 2166, 2168, 2180, 2182, 2185, 2189, 2191, 2193, 2204, 2206, 2208, 2210, 2212, 2214, 2216, 2218, 2220, 2222, 2224, 2227, 2230, 2232, 2234, 2242, 2245, 2247, 2249, 2252, 2254, 2256, 2258, 2260, 2263, 2266, 2268, 2271, 2274, 2277, 2280, 2283, 2287, 2290, 2292, 2294, 2301, 2303, 2305, 2308, 2311, 2319, 2322, 2324, 2326, 2328, 2331, 2333, 2336, 2338, 2340, 2342, 2344, 2349, 2351, 2353, 2355, 2359, 2363, 2365, 2368, 2370, 2372, 2374, 2377, 2379, 2382, 2385, 2387, 2389, 2393, 2395, 2398, 2400, 2404, 2406, 2410, 2412, 2414, 2416, 2420, 2426, 2431, 2433, 2436, 2438, 2444, 2447, 2450, 2453, 2455, 2465, 2467, 2473, 2479, 2481, 2492, 2501, 2504, 2508, 2515, 2520, 2524, 2526, 2528, 2532, 2534, 2536, 2538, 2543, 2545, 2549, 2553, 2555, 2560, 2563, 2566, 2568, 2570, 2577, 2579, 2582, 2586, 2591, 2596, 2598, 2600, 2602, 2605, 2607, 2610, 2613, 2618, 2620, 2622, 2625, 2630, 2633, 2635, 2637, 2639, 2643, 2645, 2651, 2653, 2655, 2657, 2660, 2662, 2664, 2666, 2669, 2671, 2673, 2676, 2678, 2680, 2682, 2684, 2686, 2688, 2690, 2692, 2694, 2696, 2698, 2700, 2702, 2705, 2707, 2709, 2713, 2715, 2718, 2720, 2723, 2725, 2727, 2729, 2731, 2733, 2737, 2742, 2744, 2747, 2749, 2752, 2755, 2757, 2759, 2761, 2763, 2765, 2767, 2769, 2774, 2776, 2779, 2783, 2785, 2787, 2789, 2793, 2795, 2798, 2800, 2805, 2808, 2812, 2814, 2817, 2820, 2824, 2827, 2829, 2831, 2834, 2837, 2839, 2841, 2843, 2848, 2854, 2860, 2865, 2871, 2874, 2879, 2881, 2884, 2887, 2889, 2895, 2897, 2899, 2901, 2905, 2912, 2914, 2916, or 2925 as described in more detail below. A nucleic acid also can be a fragment that is at least 40% (e.g., at least 45, 50, 55, 60, 65, 70, 75, 80, 85, 90, 95, or 99%) of the length of the full-length nucleic acid set forth in SEQ ID NOs: 1, 3, 7, 9, 11, 14, 16, 19, 21, 23, 28, 31, 33, 36, 40, 42, 44, 46, 48, 50, 52, 54, 58, 61, 66, 68, 72, 76, 78, 87, 89, 114, 116, 118, 125, 127, 130, 132, 134, 136, 138, 143, 145, 151, 153, 156, 159, 161, 164, 166, 168, 170, 172, 174, 177, 179, 181, 184, 186, 188, 190, 192, 202, 204, 206, 210, 212, 216, 221, 225, 227, 230, 241, 243, 253, 280, 283, 285, 290, 292, 296, 298, 301, 303, 305, 309, 312, 314, 321, 323, 325, 327, 329, 332, 334, 336, 339, 341, 344, 348, 352, 356, 359, 361, 366, 368, 376, 381, 388, 392, 394, 397, 399, 402, 405, 407, 413, 416, 418, 455, 458, 460, 463, 465, 471, 473, 476, 478, 481, 486, 496, 498, 500, 505, 508, 513, 519, 521, 523, 525, 527, 529, 541, 544, 549, 552, 556, 558, 561, 564, 567, 569, 571, 574, 578, 588, 594, 600, 603, 606, 608, 628, 630, 632, 639, 641, 643, 645, 647, 649, 652, 661, 668, 670, 673, 675, 678, 681, 683, 686, 688, 692, 695, 699, 701, 703, 708, 711, 713, 716, 724, 729, 736, 738, 740, 742, 748, 750, 754, 756, 758, 761, 763, 765, 767, 769, 771, 774, 777, 779, 781, 783, 785, 787, 790, 792, 797, 799, 803, 806, 808, 810, 812, 814, 816, 818, 821, 826, 829, 831, 833, 835, 837, 839, 841, 845, 847, 852, 854, 859, 862, 865, 867, 869, 872, 875, 877, 879, 881, 884, 886, 888, 891, 894, 898, 900, 904, 906, 911, 913, 918, 920, 922, 924, 927, 929, 931, 933, 938, 940, 942, 944, 946, 948, 951, 954, 956, 958, 961, 963, 966, 969, 971, 973, 975, 977, 979, 982, 984, 988, 990, 992, 994, 996, 999, 1001, 1004, 1006, 1010, 1012, 1015, 1017, 1019, 1021, 1028, 1000, 1002, 1005, 1007, 1040, 1047, 1053, 1055, 1057, 1059, 1063, 1065, 1068, 1073, 1075, 1080, 1082, 1084, 1086, 1089, 1091, 1094, 1098, 1100, 1100, 1106, 1109, 1116, 1118, 1123, 1125, 1127, 1129, 1131, 1134, 1136, 1138, 1140, 1143, 1145, 1147, 1149, 1151, 1153, 1155, 1159, 1161, 1163, 1165, 1167, 1170, 1172, 1174, 1178, 1182, 1184, 1186, 1189, 1191, 1195, 1197, 1199, 1202, 1204, 1206, 1208, 1211, 1213, 1215, 1217, 1220, 1223, 1225, 1227, 1229, 1231, 1233, 1237, 1239, 1242, 1244, 1246, 1248, 1250, 1254, 1256, 1258, 1260, 1262, 1264, 1267, 1269, 1271, 1273, 1275, 1277, 1280, 1282, 1284, 1286, 1288, 1290, 1292, 1295, 1298, 1301, 1303, 1305, 1307, 1311, 1315, 1317, 1319, 1321, 1323, 1327, 1331, 1335, 1337, 1346, 1348, 1351, 1354, 1356, 1362, 1365, 1368, 1374, 1378, 1383, 1385, 1387, 1390, 1392, 1394, 1396, 1398, 1400, 1404, 1406, 1408, 1411, 1413, 1415, 1417, 1419, 1423, 1425, 1427, 1430, 1432, 1434, 1436, 1438, 1442, 1444, 1447, 1449, 1451, 1453, 1455, 1458, 1460, 1462, 1464, 1466, 1468, 1470, 1472, 1474, 1476, 1478, 1480, 1484, 1487, 1491, 1494, 1496, 1499, 1501, 1504, 1510, 1513, 1517, 1519, 1521, 1527, 1530, 1532, 1536, 1539, 1541, 1543, 1545, 1548, 1551, 1553, 1556, 1559, 1561, 1566, 1568, 1570, 1573, 1576, 1579, 1582, 1585, 1587, 1590, 1592, 1597, 1601, 1606, 1608, 1610, 1614, 1617, 1620, 1623, 1625, 1627, 1631, 1633, 1635, 1638, 1641, 1643, 1645, 1651, 1653, 1655, 1659, 1663, 1665, 1667, 1673, 1675, 1678, 1685, 1687, 1690, 1692, 1694, 1696, 1698, 1703, 1709, 1716, 1718, 1720, 1722, 1726, 1728, 1730, 1733, 1736, 1738, 1740, 1742, 1744, 1748, 1750, 1752, 1754, 1756, 1760, 1763, 1768, 1771, 1775, 1778, 1780, 1782, 1785, 1787, 1789, 1791, 1793, 1796, 1800, 1802, 1814, 1818, 1820, 1824, 1830, 1832, 1836, 1838, 1840, 1843, 1846, 1848, 1851, 1860, 1862, 1864, 1868, 1870, 1873, 1875, 1878, 1880, 1884, 1886, 1888, 1890, 1892, 1896, 1899, 1901, 1905, 1907, 1909, 1911, 1914, 1916, 1921, 1923, 1925, 1927, 1930, 1932, 1934, 1937, 1939, 1941, 1943, 1945, 1947, 1950, 1952, 1954, 1956, 1958, 1960, 1962, 1964, 1966, 1968, 1970, 1977, 1981, 1984, 1988, 1991, 1993, 1995, 1998, 2001, 2004, 2006, 2012, 2014, 2020, 2025, 2027, 2029, 2033, 2035, 2039, 2045, 2050, 2052, 2054, 2057, 2059, 2061, 2063, 2065, 2068, 2071, 2080, 2082, 2084, 2086, 2089, 2094, 2096, 2099, 2101, 2100, 2107, 2109, 2111, 2114, 2116, 2123, 2125, 2127, 2131, 2134, 2139, 2145, 2147, 2155, 2157, 2164, 2166, 2168, 2180, 2182, 2185, 2189, 2191, 2193, 2204, 2206, 2208, 2210, 2212, 2214, 2216, 2218, 2220, 2222, 2224, 2227, 2230, 2232, 2234, 2242, 2245, 2247, 2249, 2252, 2254, 2256, 2258, 2260, 2263, 2266, 2268, 2271, 2274, 2277, 2280, 2283, 2287, 2290, 2292, 2294, 2301, 2303, 2305, 2308, 2311, 2319, 2322, 2324, 2326, 2328, 2331, 2333, 2336, 2338, 2340, 2342, 2344, 2349, 2351, 2353, 2355, 2359, 2363, 2365, 2368, 2370, 2372, 2374, 2377, 2379, 2382, 2385, 2387, 2389, 2393, 2395, 2398, 2400, 2404, 2406, 2410, 2412, 2414, 2416, 2420, 2426, 2431, 2433, 2436, 2438, 2444, 2447, 2450, 2453, 2455, 2465, 2467, 2473, 2479, 2481, 2492, 2501, 2504, 2508, 2515, 2520, 2524, 2526, 2528, 2532, 2534, 2536, 2538, 2543, 2545, 2549, 2553, 2555, 2560, 2563, 2566, 2568, 2570, 2577, 2579, 2582, 2586, 2591, 2596, 2598, 2600, 2602, 2605, 2607, 2610, 2613, 2618, 2620, 2622, 2625, 2630, 2633, 2635, 2637, 2639, 2643, 2645, 2651, 2653, 2655, 2657, 2660, 2662, 2664, 2666, 2669, 2671, 2673, 2676, 2678, 2680, 2682, 2684, 2686, 2688, 2690, 2692, 2694, 2696, 2698, 2700, 2702, 2705, 2707, 2709, 2713, 2715, 2718, 2720, 2723, 2725, 2727, 2729, 2731, 2733, 2737, 2742, 2744, 2747, 2749, 2752, 2755, 2757, 2759, 2761, 2763, 2765, 2767, 2769, 2774, 2776, 2779, 2783, 2785, 2787, 2789, 2793, 2795, 2798, 2800, 2805, 2808, 2812, 2814, 2817, 2820, 2824, 2827, 2829, 2831, 2834, 2837, 2839, 2841, 2843, 2848, 2854, 2860, 2865, 2871, 2874, 2879, 2881, 2884, 2887, 2889, 2895, 2897, 2899, 2901, 2905, 2912, 2914, 2916, or 2925.

A biomass-modulating nucleic acid can comprise the nucleotide sequence set forth in SEQ ID NO:2879. Alternatively, a biomass-modulating nucleic acid can be a variant of the nucleic acid having the nucleotide sequence set forth in SEQ ID NO:2879. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:2879.

A biomass-modulating nucleic acid can comprise the nucleotide sequence set forth in SEQ ID NO:309. Alternatively, a biomass-modulating nucleic acid can be a variant of the nucleic acid having the nucleotide sequence set forth in SEQ ID NO:309. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO: 309.

A biomass-modulating nucleic acid can comprise the nucleotide sequence set forth in SEQ ID NO:402. Alternatively, a biomass-modulating nucleic acid can be a variant of the nucleic acid having the nucleotide sequence set forth in SEQ ID NO:402. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:402.

A biomass-modulating nucleic acid can comprise the nucleotide sequence set forth in SEQ ID NO:668. Alternatively, a biomass-modulating nucleic acid can be a variant of the nucleic acid having the nucleotide sequence set forth in SEQ ID NO:668. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:668.

A biomass-modulating nucleic acid can comprise the nucleotide sequence set forth in SEQ ID NO:1796. Alternatively, a biomass-modulating nucleic acid can be a variant of the nucleic acid having the nucleotide sequence set forth in SEQ ID NO:1796. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:1796.

A biomass-modulating nucleic acid can comprise the nucleotide sequence set forth in SEQ ID NO:716. Alternatively, a biomass-modulating nucleic acid can be a variant of the nucleic acid having the nucleotide sequence set forth in SEQ ID NO:716. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:716.

A biomass-modulating nucleic acid can comprise the nucleotide sequence set forth in SEQ ID NO:692. Alternatively, a biomass-modulating nucleic acid can be a variant of the nucleic acid having the nucleotide sequence set forth in SEQ ID NO:692. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:692.

A biomass-modulating nucleic acid can comprise the nucleotide sequence set forth in SEQ ID NO:1517. Alternatively, a biomass-modulating nucleic acid can be a variant of the nucleic acid having the nucleotide sequence set forth in SEQ ID NO:1517. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:1517.

A biomass-modulating nucleic acid can comprise the nucleotide sequence set forth in SEQ ID NO:1623. Alternatively, a biomass-modulating nucleic acid can be a variant of the nucleic acid having the nucleotide sequence set forth in SEQ ID NO:1623. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:1623.

A biomass-modulating nucleic acid can comprise the nucleotide sequence set forth in SEQ ID NO:1886. Alternatively, a biomass-modulating nucleic acid can be a variant of the nucleic acid having the nucleotide sequence set forth in SEQ ID NO:1886. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:1886.

A biomass-modulating nucleic acid can comprise the nucleotide sequence set forth in SEQ ID NO:833. Alternatively, a biomass-modulating nucleic acid can be a variant of the nucleic acid having the nucleotide sequence set forth in SEQ ID NO:833. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:833.

A biomass-modulating nucleic acid can comprise the nucleotide sequence set forth in SEQ ID NO:1317. Alternatively, a biomass-modulating nucleic acid can be a variant of the nucleic acid having the nucleotide sequence set forth in SEQ ID NO:1317. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:1317.

A biomass-modulating nucleic acid can comprise the nucleotide sequence set forth in SEQ ID NO:458. Alternatively, a biomass-modulating nucleic acid can be a variant of the nucleic acid having the nucleotide sequence set forth in SEQ ID NO:458. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:458.

A biomass-modulating nucleic acid can comprise the nucleotide sequence set forth in SEQ ID NO:334. Alternatively, a biomass-modulating nucleic acid can be a variant of the nucleic acid having the nucleotide sequence set forth in SEQ ID NO:334. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:334.

A biomass-modulating nucleic acid can comprise the nucleotide sequence set forth in SEQ ID NO:845. Alternatively, a biomass-modulating nucleic acid can be a variant of the nucleic acid having the nucleotide sequence set forth in SEQ ID NO:845. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:845.

A biomass-modulating nucleic acid can comprise the nucleotide sequence set forth in SEQ ID NO:1425. Alternatively, a biomass-modulating nucleic acid can be a variant of the nucleic acid having the nucleotide sequence set forth in SEQ ID NO:1425. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:1425.

A biomass-modulating nucleic acid can comprise the nucleotide sequence set forth in SEQ ID NO:2524. Alternatively, a biomass-modulating nucleic acid can be a variant of the nucleic acid having the nucleotide sequence set forth in SEQ ID NO:2524. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:2524.

A biomass-modulating nucleic acid can comprise the nucleotide sequence set forth in SEQ ID NO:1271. Alternatively, a biomass-modulating nucleic acid can be a variant of the nucleic acid having the nucleotide sequence set forth in SEQ ID NO:1271. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:1271.

A biomass-modulating nucleic acid can comprise the nucleotide sequence set forth in SEQ ID NO:877. Alternatively, a biomass-modulating nucleic acid can be a variant of the nucleic acid having the nucleotide sequence set forth in SEQ ID NO:877. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:877.

A biomass-modulating nucleic acid can comprise the nucleotide sequence set forth in SEQ ID NO:1659. Alternatively, a biomass-modulating nucleic acid can be a variant of the nucleic acid having the nucleotide sequence set forth in SEQ ID NO:1659. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:1659.

A biomass-modulating nucleic acid can comprise the nucleotide sequence set forth in SEQ ID NO:2653. Alternatively, a biomass-modulating nucleic acid can be a variant of the nucleic acid having the nucleotide sequence set forth in SEQ ID NO:2653. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:2653.

A biomass-modulating nucleic acid can comprise the nucleotide sequence set forth in SEQ ID NO:1138. Alternatively, a biomass-modulating nucleic acid can be a variant of the nucleic acid having the nucleotide sequence set forth in SEQ ID NO:1136. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:1136.

A biomass-modulating nucleic acid can comprise the nucleotide sequence set forth in SEQ ID NO:2665. Alternatively, a biomass-modulating nucleic acid can be a variant of the nucleic acid having the nucleotide sequence set forth in SEQ ID NO:2665. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:2665.

A biomass-modulating nucleic acid can comprise the nucleotide sequence set forth in SEQ ID NO:1106. Alternatively, a biomass-modulating nucleic acid can be a variant of the nucleic acid having the nucleotide sequence set forth in SEQ ID NO:1106. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:1106.

A biomass-modulating nucleic acid can comprise the nucleotide sequence set forth in SEQ ID NO:1945. Alternatively, a biomass-modulating nucleic acid can be a variant of the nucleic acid having the nucleotide sequence set forth in SEQ ID NO:1945. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:1945.

A biomass-modulating nucleic acid can comprise the nucleotide sequence set forth in SEQ ID NO:1053. Alternatively, a biomass-modulating nucleic acid can be a variant of the nucleic acid having the nucleotide sequence set forth in SEQ ID NO:1053. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:1053.

A biomass-modulating nucleic acid can comprise the nucleotide sequence set forth in SEQ ID NO:114. Alternatively, a biomass-modulating nucleic acid can be a variant of the nucleic acid having the nucleotide sequence set forth in SEQ ID NO:114. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:114.

A biomass-modulating nucleic acid can comprise the nucleotide sequence set forth in SEQ ID NO:2707. Alternatively, a biomass-modulating nucleic acid can be a variant of the nucleic acid having the nucleotide sequence set forth in SEQ ID NO:2707. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:2707.

A biomass-modulating nucleic acid can comprise the nucleotide sequence set forth in SEQ ID NO:1480. Alternatively, a biomass-modulating nucleic acid can be a variant of the nucleic acid having the nucleotide sequence set forth in SEQ ID NO:1480. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:1480.

A biomass-modulating nucleic acid can comprise the nucleotide sequence set forth in SEQ ID NO:2747. Alternatively, a biomass-modulating nucleic acid can be a variant of the nucleic acid having the nucleotide sequence set forth in SEQ ID NO:2747. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:2747.

A biomass-modulating nucleic acid can comprise the nucleotide sequence set forth in SEQ ID NO:946. Alternatively, a biomass-modulating nucleic acid can be a variant of the nucleic acid having the nucleotide sequence set forth in SEQ ID NO:946. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:946.

A biomass-modulating nucleic acid can comprise the nucleotide sequence set forth in SEQ ID NO:1726. Alternatively, a biomass-modulating nucleic acid can be a variant of the nucleic acid having the nucleotide sequence set forth in SEQ ID NO:1726. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:1726.

A biomass-modulating nucleic acid can comprise the nucleotide sequence set forth in SEQ ID NO:2145. Alternatively, a biomass-modulating nucleic acid can be a variant of the nucleic acid having the nucleotide sequence set forth in SEQ ID NO:2145. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:2145.

A biomass-modulating nucleic acid can comprise the nucleotide sequence set forth in SEQ ID NO:2618. Alternatively, a biomass-modulating nucleic acid can be a variant of the nucleic acid having the nucleotide sequence set forth in SEQ ID NO:2618. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:2618.

A biomass-modulating nucleic acid can comprise the nucleotide sequence set forth in SEQ ID NO:210. Alternatively, a biomass-modulating nucleic acid can be a variant of the nucleic acid having the nucleotide sequence set forth in SEQ ID NO:210. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:210.

A biomass-modulating nucleic acid can comprise the nucleotide sequence set forth in SEQ ID NO:628. Alternatively, a biomass-modulating nucleic acid can be a variant of the nucleic acid having the nucleotide sequence set forth in SEQ ID NO:628. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:628.

A biomass-modulating nucleic acid can comprise the nucleotide sequence set forth in SEQ ID NO:2543. Alternatively, a biomass-modulating nucleic acid can be a variant of the nucleic acid having the nucleotide sequence set forth in SEQ ID NO:2543. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:2543.

A biomass-modulating nucleic acid can comprise the nucleotide sequence set forth in SEQ ID NO:2598. Alternatively, a biomass-modulating nucleic acid can be a variant of the nucleic acid having the nucleotide sequence set forth in SEQ ID NO:2598. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:2598.

A biomass-modulating nucleic acid can comprise the nucleotide sequence set forth in SEQ ID NO:2436. Alternatively, a biomass-modulating nucleic acid can be a variant of the nucleic acid having the nucleotide sequence set forth in SEQ ID NO:2436. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:2436.

A biomass-modulating nucleic acid can comprise the nucleotide sequence set forth in SEQ ID NO:2089. Alternatively, a biomass-modulating nucleic acid can be a variant of the nucleic acid having the nucleotide sequence set forth in SEQ ID NO:2089. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:2089.

A biomass-modulating nucleic acid can comprise the nucleotide sequence set forth in SEQ ID NO:2322. Alternatively, a biomass-modulating nucleic acid can be a variant of the nucleic acid having the nucleotide sequence set forth in SEQ ID NO:2322. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:2322.

A biomass-modulating nucleic acid can comprise the nucleotide sequence set forth in SEQ ID NO:1. Alternatively, a biomass-modulating nucleic acid can be a variant of the nucleic acid having the nucleotide sequence set forth in SEQ ID NO:1. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:1.

A biomass-modulating nucleic acid can comprise the nucleotide sequence set forth in SEQ ID NO:2027. Alternatively, a biomass-modulating nucleic acid can be a variant of the nucleic acid having the nucleotide sequence set forth in SEQ ID NO:2027. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:2027.

A biomass-modulating nucleic acid can comprise the nucleotide sequence set forth in SEQ ID NO:1568. Alternatively, a biomass-modulating nucleic acid can be a variant of the nucleic acid having the nucleotide sequence set forth in SEQ ID NO:1568. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:1568.

A biomass-modulating nucleic acid can comprise the nucleotide sequence set forth in SEQ ID NO:500. Alternatively, a biomass-modulating nucleic acid can be a variant of the nucleic acid having the nucleotide sequence set forth in SEQ ID NO:500. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:500.

A biomass-modulating nucleic acid can comprise the nucleotide sequence set forth in SEQ ID NO:2189. Alternatively, a biomass-modulating nucleic acid can be a variant of the nucleic acid having the nucleotide sequence set forth in SEQ ID NO:2189. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:2189.

A biomass-modulating nucleic acid can comprise the nucleotide sequence set forth in SEQ ID NO:748. Alternatively, a biomass-modulating nucleic acid can be a variant of the nucleic acid having the nucleotide sequence set forth in SEQ ID NO:748. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:748.

A biomass-modulating nucleic acid can comprise the nucleotide sequence set forth in SEQ ID NO:78. Alternatively, a biomass-modulating nucleic acid can be a variant of the nucleic acid having the nucleotide sequence set forth in SEQ ID NO:78. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:78.

A biomass-modulating nucleic acid can comprise the nucleotide sequence set forth in SEQ ID NO:283. Alternatively, a biomass-modulating nucleic acid can be a variant of the nucleic acid having the nucleotide sequence set forth in SEQ ID NO:283. For example, a biomass-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:283.

Isolated nucleic acid molecules can be produced by standard techniques. For example, polymerase chain reaction (PCR) techniques can be used to obtain an isolated nucleic acid containing a nucleotide sequence described herein. PCR can be used to amplify specific sequences from DNA as well as RNA, including sequences from total genomic DNA or total cellular RNA. Various PCR methods are described, for example, in PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid. Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids of the invention also can be obtained by mutagenesis of, e.g., a naturally occurring DNA.

B. Use of Nucleic Acids to Modulate Expression of Polypeptides

i. Expression of a Biomass-Modulating Polypeptide

A nucleic acid encoding one of the biomass-modulating polypeptides described herein can be used to express the polypeptide in a plant species of interest, typically by transforming a plant cell with a nucleic acid having the coding sequence for the polypeptide operably linked in sense orientation to one or more regulatory regions. It will be appreciated that because of the degeneracy of the genetic code, a number of nucleic acids can encode a particular biomass-modulating polypeptide; i.e., for many amino acids, there is more than one nucleotide triplet that serves as the codon for the amino acid. Thus, codons in the coding sequence for a given biomass-modulating polypeptide can be modified such that optimal expression in a particular plant species is obtained, using appropriate codon bias tables for that species.

In some cases, expression of a biomass-modulating polypeptide inhibits one or more functions of an endogenous polypeptide. For example, a nucleic acid that encodes a dominant negative polypeptide can be used to inhibit protein function. A dominant negative polypeptide typically is mutated or truncated relative to an endogenous wild type polypeptide, and its presence in a cell inhibits one or more functions of the wild type polypeptide in that cell, i.e., the dominant negative polypeptide is genetically dominant and confers a loss of function. The mechanism by which a dominant negative polypeptide confers such a phenotype can vary but often involves a protein-protein interaction or a protein-DNA interaction. For example, a dominant negative polypeptide can be an enzyme that is truncated relative to a native wild type enzyme, such that the truncated polypeptide retains domains involved in binding a first protein but lacks domains involved in binding a second protein. The truncated polypeptide is thus unable to properly modulate the activity of the second protein. See, e.g., US 2007/0056058. As another example, a point mutation that results in a non-conservative amino acid substitution in a catalytic domain can result in a dominant negative polypeptide. See, e.g., US 2005/032221. As another example, a dominant negative polypeptide can be a transcription factor that is truncated relative to a native wild type transcription factor, such that the truncated polypeptide retains the DNA binding domain(s) but lacks the activation domain(s). Such a truncated polypeptide can inhibit the wild type transcription factor from binding DNA, thereby inhibiting transcription activation.

ii. Inhibition of Expression of a Biomass-Modulating Polypeptide

Polynucleotides and recombinant constructs described herein can be used to inhibit expression of a biomass-modulating polypeptide in a plant species of interest. See, e.g., Matzke and Birchler, Nature Reviews Genetics 6:24-35 (2005); Akashi et al., Nature Reviews Mol. Cell. Biology 6:413-422 (2005); Mittal, Nature Reviews Genetics 5:355-365 (2004); and Nature Reviews RNA interference collection, Oct. 2005 on the World Wide Web at nature.com/reviews/focus/mai. A number of nucleic acid based methods, including antisense RNA, ribozyme directed RNA cleavage, post-transcriptional gene silencing (PTGS), e.g., RNA interference (RNAi), and transcriptional gene silencing (TGS) are known to inhibit gene expression in plants. Suitable polynucleotides include full-length nucleic acids encoding biomass-modulating polypeptides or fragments of such full-length nucleic acids. In some embodiments, a complement of the full-length nucleic acid or a fragment thereof can be used. Typically, a fragment is at least 10 nucleotides, e.g., at least 12, 13, 14, 15, 16, 17, 18, 19, 20, 21, 22, 23, 24, 25, 26, 27, 30, 35, 40, 50, 80, 100, 200, 500 nucleotides or more. Generally, higher homology can be used to compensate for the use of a shorter sequence.

Antisense technology is one well-known method. In this method, a nucleic acid of a gene to be repressed is cloned and operably linked to a regulatory region and a transcription termination sequence so that the antisense strand of RNA is transcribed. The recombinant construct is then transformed into plants, as described herein, and the antisense strand of RNA is produced. The nucleic acid need not be the entire sequence of the gene to be repressed, but typically will be substantially complementary to at least a portion of the sense strand of the gene to be repressed.

In another method, a nucleic acid can be transcribed into a ribozyme, or catalytic RNA, that affects expression of an mRNA. See, U.S. Pat. No. 6,423,885. Ribozymes can be designed to specifically pair with virtually any target RNA and cleave the phosphodiester backbone at a specific location, thereby functionally inactivating the target RNA. Heterologous nucleic acids can encode ribozymes designed to cleave particular mRNA transcripts, thus preventing expression of a polypeptide. Hammerhead ribozymes are useful for destroying particular mRNAs, although various ribozymes that cleave mRNA at site-specific recognition sequences can be used. Hammerhead ribozymes cleave mRNAs at locations dictated by flanking regions that form complementary base pairs with the target mRNA. The sole requirement is that the target RNA contains a 5′-UG-3′ nucleotide sequence. The construction and production of hammerhead ribozymes is known in the art. See, for example, U.S. Pat. No. 5,254,678 and WO 02/46449 and references cited therein. Hammerhead ribozyme sequences can be embedded in a stable RNA such as a transfer RNA (tRNA) to increase cleavage efficiency in vivo. Perriman et al., Proc. Natl. Acad. Sci. USA, 92(13):6175-6179 (1995); de Feyter and Gaudron, Methods in Molecular Biology, Vol. 74, Chapter 43, “Expressing Ribozymes in Plants”, Edited by Turner, P.C., Humana Press Inc., Totowa, N.J. RNA endoribonucleases which have been described, such as the one that occurs naturally in Tetrahymena thermophila, can be useful. See, for example, U.S. Pat. Nos. 4,987,071 and 6,423,885.

PTGS, e.g., RNAi, can also be used to inhibit the expression of a gene. For example, a construct can be prepared that includes a sequence that is transcribed into an RNA that can anneal to itself, e.g., a double stranded RNA having a stem-loop structure. In some embodiments, one strand of the stem portion of a double stranded RNA comprises a sequence that is similar or identical to the sense coding sequence or a fragment thereof of a biomass-modulating polypeptide, and that is from about 10 nucleotides to about 2,500 nucleotides in length. The length of the sequence that is similar or identical to the sense coding sequence can be from 10 nucleotides to 500 nucleotides, from 15 nucleotides to 300 nucleotides, from 20 nucleotides to 100 nucleotides, or from 25 nucleotides to 100 nucleotides. The other strand of the stem portion of a double stranded RNA comprises a sequence that is similar or identical to the antisense strand or a fragment thereof of the coding sequence of the biomass-modulating polypeptide, and can have a length that is shorter, the same as, or longer than the corresponding length of the sense sequence. In some cases, one strand of the stem portion of a double stranded RNA comprises a sequence that is similar or identical to the 3′ or 5′ untranslated region, or a fragment thereof, of an mRNA encoding a biomass-modulating polypeptide, and the other strand of the stem portion of the double stranded RNA comprises a sequence that is similar or identical to the sequence that is complementary to the 3′ or 5′ untranslated region, respectively, or a fragment thereof, of the mRNA encoding the biomass-modulating polypeptide. In other embodiments, one strand of the stem portion of a double stranded RNA comprises a sequence that is similar or identical to the sequence of an intron, or a fragment thereof, in the pre-mRNA encoding a biomass-modulating polypeptide, and the other strand of the stem portion comprises a sequence that is similar or identical to the sequence that is complementary to the sequence of the intron, or a fragment thereof, in the pre-mRNA.

The loop portion of a double stranded RNA can be from 3 nucleotides to 5,000 nucleotides, e.g., from 3 nucleotides to 25 nucleotides, from 15 nucleotides to 1,000 nucleotides, from 20 nucleotides to 500 nucleotides, or from 25 nucleotides to 200 nucleotides. The loop portion of the RNA can include an intron or a fragment thereof. A double stranded RNA can have zero, one, two, three, four, five, six, seven, eight, nine, ten, or more stem-loop structures.

A construct including a sequence that is operably linked to a regulatory region and a transcription termination sequence, and that is transcribed into an RNA that can form a double stranded RNA, is transformed into plants as described herein. Methods for using RNAi to inhibit the expression of a gene are known to those of skill in the art. See, e.g., U.S. Pat. Nos. 5,034,323; 6,326,527; 6,452,067; 6,573,099; 6,753,139; and 6,777,588. See also WO 97/01952; WO 98/53083; WO 99/32619; WO 98/36083; and U.S. Patent Publications 20030175965, 20030175783, 20040214330, and 20030180945.

Constructs containing regulatory regions operably linked to nucleic acid molecules in sense orientation can also be used to inhibit the expression of a gene. The transcription product can be similar or identical to the sense coding sequence, or a fragment thereof, of a biomass-modulating polypeptide. The transcription product also can be unpolyadenylated, lack a 5′ cap structure, or contain an unspliceable intron. Methods of inhibiting gene expression using a full-length cDNA as well as a partial cDNA sequence are known in the art. See, e.g., U.S. Pat. No. 5,231,020.

In some embodiments, a construct containing a nucleic acid having at least one strand that is a template for both sense and antisense sequences that are complementary to each other is used to inhibit the expression of a gene. The sense and antisense sequences can be part of a larger nucleic acid molecule or can be part of separate nucleic acid molecules having sequences that are not complementary. The sense or antisense sequence can be a sequence that is identical or complementary to the sequence of an mRNA, the 3′ or 5′ untranslated region of an mRNA, or an intron in a pre-mRNA encoding a biomass-modulating polypeptide, or a fragment of such sequences. In some embodiments, the sense or antisense sequence is identical or complementary to a sequence of the regulatory region that drives transcription of the gene encoding a biomass-modulating polypeptide. In each case, the sense sequence is the sequence that is complementary to the antisense sequence.

The sense and antisense sequences can be a length greater than about 10 nucleotides (e.g., 12, 13, 14, 15, 16, 17, 18, 19, 20, 21, 22, 23, 24, 25, 26, 27, 28, 29, 30, or more nucleotides). For example, an antisense sequence can be 21 or 22 nucleotides in length. Typically, the sense and antisense sequences range in length from about 15 nucleotides to about 30 nucleotides, e.g., from about 18 nucleotides to about 28 nucleotides, or from about 21 nucleotides to about 25 nucleotides.

In some embodiments, an antisense sequence is a sequence complementary to an mRNA sequence, or a fragment thereof, encoding a biomass-modulating polypeptide described herein. The sense sequence complementary to the antisense sequence can be a sequence present within the mRNA of the biomass-modulating polypeptide. Typically, sense and antisense sequences are designed to correspond to a 15-30 nucleotide sequence of a target mRNA such that the level of that target mRNA is reduced.

In some embodiments, a construct containing a nucleic acid having at least one strand that is a template for more than one sense sequence (e.g., 2, 3, 4, 5, 6, 7, 8, 9, 10 or more sense sequences) can be used to inhibit the expression of a gene. Likewise, a construct containing a nucleic acid having at least one strand that is a template for more than one antisense sequence (e.g., 2, 3, 4, 5, 6, 7, 8, 9, 10 or more antisense sequences) can be used to inhibit the expression of a gene. For example, a construct can contain a nucleic acid having at least one strand that is a template for two sense sequences and two antisense sequences. The multiple sense sequences can be identical or different, and the multiple antisense sequences can be identical or different. For example, a construct can have a nucleic acid having one strand that is a template for two identical sense sequences and two identical antisense sequences that are complementary to the two identical sense sequences. Alternatively, an isolated nucleic acid can have one strand that is a template for (1) two identical sense sequences 20 nucleotides in length, (2) one antisense sequence that is complementary to the two identical sense sequences 20 nucleotides in length, (3) a sense sequence 30 nucleotides in length, and (4) three identical antisense sequences that are complementary to the sense sequence 30 nucleotides in length. The constructs provided herein can be designed to have a suitable arrangement of sense and antisense sequences. For example, two identical sense sequences can be followed by two identical antisense sequences or can be positioned between two identical antisense sequences.

A nucleic acid having at least one strand that is a template for one or more sense and/or antisense sequences can be operably linked to a regulatory region to drive transcription of an RNA molecule containing the sense and/or antisense sequence(s). In addition, such a nucleic acid can be operably linked to a transcription terminator sequence, such as the terminator of the nopaline synthase (nos) gene. In some cases, two regulatory regions can direct transcription of two transcripts: one from the top strand, and one from the bottom strand. See, for example, Yan et al., Plant Physiol., 141:1508-1518 (2006). The two regulatory regions can be the same or different. The two transcripts can form double-stranded RNA molecules that induce degradation of the target RNA. In some cases, a nucleic acid can be positioned within a T-DNA or plant-derived transfer DNA (P-DNA) such that the left and right T-DNA border sequences or the left and right border-like sequences of the P-DNA flank, or are on either side of, the nucleic acid. See, US 2006/0265788. The nucleic acid sequence between the two regulatory regions can be from about 15 to about 300 nucleotides in length. In some embodiments, the nucleic acid sequence between the two regulatory regions is from about 15 to about 200 nucleotides in length, from about 15 to about 100 nucleotides in length, from about 15 to about 50 nucleotides in length, from about 18 to about 50 nucleotides in length, from about 18 to about 40 nucleotides in length, from about 18 to about 30 nucleotides in length, or from about 18 to about 25 nucleotides in length.

In some nucleic-acid based methods for inhibition of gene expression in plants, a suitable nucleic acid can be a nucleic acid analog. Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine, and 5-methyl-2′-deoxycytidine and 5-bromo-2′-deoxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six-membered morpholino ring, or peptide nucleic acids, in which the deoxyphosphate backbone is replaced by a pseudopeptide backbone and the four bases are retained. See, for example, Summerton and Weller, Antisense Nucleic Acid Drug Dev., 7:187-195 (1997); Hyrup et al., Bioorgan. Med. Chem., 4:5-23 (1996). In addition, the deoxyphosphate backbone can be replaced with, for example, a phosphorothioate or phosphorodithioate backbone, a phosphoroamidite, or an alkyl phosphotriester backbone.

C. Constructs/Vectors

Recombinant constructs provided herein can be used to transform plants or plant cells in order to modulate biomass levels. A recombinant nucleic acid construct can comprise a nucleic acid encoding a biomass-modulating polypeptide as described herein, operably linked to a regulatory region suitable for expressing the biomass-modulating polypeptide in the plant or cell. Thus, a nucleic acid can comprise a coding sequence that encodes a biomass-modulating polypeptides as set forth in SEQ ID NOs: 2, 4, 5, 6, 8, 10, 12, 13, 15, 17, 18, 20, 22, 24, 25, 26, 27, 29, 30, 32, 34, 35, 37, 38, 39, 41, 43, 45, 47, 49, 51, 53, 55, 56, 57, 59, 60, 62, 63, 64, 65, 67, 69, 70, 71, 73, 74, 75, 77, 79, 80, 81, 82, 83, 84, 85, 86, 88, 90, 91, 92, 93, 94, 95, 96, 97, 98, 99, 100, 101, 102, 100, 104, 105, 106, 107, 108, 109, 110, 111, 112, 113, 115, 117, 119, 120, 121, 122, 123, 124, 126, 128, 129, 131, 133, 135, 137, 139, 140, 141, 142, 144, 146, 147, 148, 149, 150, 152, 154, 155, 157, 158, 160, 162, 163, 165, 167, 169, 171, 173, 175, 176, 178, 180, 182, 183, 185, 187, 189, 191, 193, 194, 195, 196, 197, 198, 199, 200, 201, 203, 205, 207, 208, 209, 211, 213, 214, 215, 217, 218, 219, 220, 222, 223, 224, 226, 228, 229, 231, 232, 233, 234, 235, 236, 237, 238, 239, 240, 242, 244, 245, 246, 247, 248, 249, 250, 251, 252, 254, 255, 256, 257, 258, 259, 260, 261, 262, 263, 264, 265, 266, 267, 268, 269, 270, 271, 272, 273, 274, 275, 276, 277, 278, 279, 281, 282, 284, 286, 287, 288, 289, 291, 293, 294, 295, 297, 299, 300, 302, 304, 306, 307, 308, 310, 311, 313, 315, 316, 317, 318, 319, 320, 322, 324, 326, 328, 330, 331, 333, 335, 337, 338, 340, 342, 343, 345, 346, 347, 349, 350, 351, 353, 354, 355, 357, 358, 360, 362, 363, 364, 365, 367, 369, 370, 371, 372, 373, 374, 375, 377, 378, 379, 380, 382, 383, 384, 385, 386, 387, 389, 390, 391, 393, 395, 396, 398, 400, 401, 403, 404, 406, 408, 409, 410, 411, 412, 414, 415, 417, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 456, 457, 459, 461, 462, 464, 466, 467, 468, 469, 470, 472, 474, 475, 477, 479, 480, 482, 483, 484, 485, 487, 488, 489, 490, 491, 492, 493, 494, 495, 497, 499, 501, 502, 503, 504, 506, 507, 509, 510, 511, 512, 514, 515, 516, 517, 518, 520, 522, 524, 526, 528, 530, 531, 532, 533, 534, 535, 536, 537, 538, 539, 540, 542, 543, 545, 546, 547, 548, 550, 551, 553, 554, 555, 557, 559, 560, 562, 563, 565, 566, 568, 570, 572, 573, 575, 576, 577, 579, 580, 581, 582, 583, 584, 585, 586, 587, 589, 590, 591, 592, 593, 595, 596, 597, 598, 599, 601, 602, 604, 605, 607, 609, 610, 611, 612, 613, 614, 615, 616, 617, 618, 619, 620, 621, 622, 623, 624, 625, 626, 627, 629, 631, 633, 634, 635, 36, 637, 638, 640, 642, 644, 646, 648, 650, 651, 653, 654, 655, 656, 657, 658, 659, 660, 662, 663, 664, 665, 666, 667, 669, 671, 672, 674, 676, 677, 679, 680, 682, 684, 685, 687, 689, 690, 691, 693, 694, 696, 697, 698, 00, 702, 704, 705, 706, 707, 709, 710, 712, 714, 715, 717, 718, 719, 720, 721, 722, 723, 725, 726, 727, 728, 730, 731, 732, 733, 734, 735, 737, 739, 741, 743, 744, 745, 746, 747, 749, 751, 752, 753, 755, 757, 759, 760, 762, 764, 766, 768, 770, 772, 773, 775, 776, 778, 780, 782, 784, 786, 788, 789, 791, 793, 794, 795, 796, 798, 800, 801, 802, 804, 805, 807, 809, 811, 813, 815, 817, 819, 820, 822, 823, 824, 825, 827, 828, 830, 832, 834, 836, 838, 840, 842, 843, 844, 846, 848, 849, 850, 851, 853, 855, 856, 857, 858, 860, 861, 863, 864, 866, 868, 870, 871, 873, 874, 876, 878, 880, 882, 883, 885, 887, 889, 890, 892, 893, 895, 896, 897, 899, 901, 902, 903, 905, 907, 908, 909, 910, 912, 914, 915, 916, 917, 919, 921, 923, 925, 926, 928, 930, 932, 934, 935, 936, 937, 939, 941, 943, 945, 947, 949, 950, 952, 953, 955, 957, 959, 960, 962, 964, 965, 967, 968, 970, 972, 974, 976, 978, 980, 981, 983, 985, 86, 87, 989, 991, 993, 995, 997, 998, 1000, 1002, 1003, 1005, 1007, 1008, 1009, 1011, 1013, 1014, 1016, 1018, 1020, 1022, 1023, 1024, 1025, 1026, 1027, 1029, 1001, 1033, 1034, 1036, 1038, 1039, 1041, 1042, 1043, 1044, 1045, 1046, 1048, 1049, 1050, 1051, 1052, 1054, 1056, 1058, 1060, 1061, 1062, 1064, 1066, 1067, 1069, 1070, 1071, 1072, 1074, 1076, 1077, 1078, 1079, 1081, 1083, 1085, 1087, 1088, 1090, 1092, 1093, 1095, 1096, 1097, 1099, 1101, 1102, 1104, 1105, 1107, 1108, 1110, 1111, 1112, 1113, 1114, 1115, 1117, 1119, 1120, 1121, 1122, 1124, 1126, 1128, 1130, 1132, 1133, 1135, 1137, 1139, 1141, 1142, 1144, 1146, 1148, 1150, 1152, 1154, 1156, 1157, 1158, 1160, 1162, 1164, 1166, 1168, 1169, 1171, 1173, 1175, 1176, 1177, 1179, 1180, 1181, 1183, 1185, 1187, 1188, 1190, 1192, 1193, 1194, 1196, 1198, 1200, 1201, 1203, 1205, 1207, 1209, 1210, 1212, 1214, 1216, 1218, 1219, 1221, 1222, 1224, 1226, 1228, 1230, 1232, 1234, 1235, 1236, 1238, 1240, 1241, 1243, 1245, 1247, 1249, 1251, 1252, 1253, 1255, 1257, 1259, 1261, 1263, 1265, 1266, 1268, 1270, 1272, 1274, 1276, 1278, 1279, 1281, 1283, 1285, 1287, 1289, 1291, 1293, 1294, 1296, 1297, 1299, 1300, 1302, 1304, 1306, 1308, 1309, 1310, 1312, 1313, 1314, 1316, 1318, 1320, 1322, 1324, 1325, 1326, 1328, 1329, 1330, 1332, 1333, 1334, 1336, 1338, 1339, 1340, 1341, 1342, 1343, 1344, 1345, 1347, 1349, 1350, 1352, 1353, 1355, 1357, 1358, 1359, 1360, 1361, 1363, 1364, 1366, 1367, 1369, 1370, 1371, 1372, 1373, 1375, 1376, 1377, 1379, 1380, 1381, 1382, 1384, 1386, 1388, 1389, 1391, 1393, 1395, 1397, 1399, 1401, 1402, 1403, 1405, 1407, 1409, 1410, 1412, 1414, 1416, 1418, 1420, 1421, 1422, 1424, 1426, 1428, 1429, 1431, 1433, 1435, 1437, 1439, 1440, 1441, 1443, 1445, 1446, 1448, 1450, 1452, 1454, 1456, 1457, 1459, 1461, 1463, 1465, 1467, 1469, 1471, 1473, 1475, 1477, 1479, 1481, 1482, 1483, 1485, 1486, 1488, 1489, 1490, 1492, 1493, 1495, 1497, 1498, 1500, 1502, 1503, 1505, 1506, 1507, 1508, 1509, 1511, 1512, 1514, 1515, 1516, 1518, 1520, 1522, 1523, 1524, 1525, 1526, 1528, 1529, 1531, 1533, 1534, 1535, 1537, 1538, 1540, 1542, 1544, 1546, 1547, 1549, 1550, 1552, 1554, 1555, 1557, 1558, 1560, 1562, 1563, 1564, 1565, 1568, 1569, 1571, 1572, 1574, 1575, 1577, 1578, 1580, 1581, 1583, 1584, 1586, 1588, 1589, 1591, 1593, 1594, 1595, 1596, 1598, 1599, 1600, 1602, 1603, 1604, 1605, 1607, 1609, 1611, 1612, 1613, 1615, 1616, 1618, 1619, 1621, 1622, 1624, 1626, 1628, 1629, 1630, 1632, 1634, 1636, 1637, 1639, 1640, 1642, 1644, 1646, 1647, 1648, 1649, 1650, 1652, 1654, 1656, 1657, 1658, 1660, 1661, 1662, 1664, 1666, 1668, 1669, 1670, 1671, 1672, 1674, 1676, 1677, 1679, 1680, 1681, 1682, 1683, 1684, 1686, 1688, 1689, 1691, 1693, 1695, 1697, 1699, 1700, 1701, 1702, 1704, 1705, 1706, 1707, 1708, 1710, 1711, 1712, 1713, 1714, 1715, 1717, 1719, 1721, 1723, 1724, 1725, 1727, 1729, 1731, 1732, 1734, 1735, 1737, 1739, 1741, 1743, 1745, 1746, 1747, 1749, 1751, 1753, 1755, 1757, 1758, 1759, 1761, 1762, 1764, 1765, 1766, 1767, 1769, 1770, 1772, 1773, 1774, 1776, 1777, 1779, 1781, 1783, 1784, 1786, 1788, 1790, 1792, 1794, 1795, 1797, 798, 1799, 1801, 1803, 1804, 1805, 1806, 1807, 1808, 1809, 1810, 1811, 1812, 1813, 1815, 1816, 1817, 1819, 1821, 1822, 1823, 1825, 1826, 1827, 1828, 1829, 1831, 1833, 1834, 1835, 1837, 1839, 1841, 1842, 1844, 1845, 1847, 1849, 1850, 1852, 1853, 1854, 1855, 1856, 1857, 1858, 1859, 1861, 1863, 1865, 1866, 1867, 1869, 1871, 1872, 1874, 1876, 1877, 1879, 1881, 1882, 1883, 1885, 1887, 1889, 1891, 1893, 1894, 1895, 1897, 1898, 1900, 1902, 1903, 1904, 1906, 1908, 1910, 1912, 1913, 1915, 1917, 1918, 1919, 1920, 1922, 1924, 1926, 1928, 1929, 1931, 1933, 1935, 1936, 1938, 1940, 1942, 1944, 1946, 1948, 1949, 1951, 1953, 1955, 1957, 1959, 1961, 1963, 1965, 1967, 1969, 1971, 1972, 1973, 1974, 1975, 1976, 1978, 1979, 1980, 1982, 1983, 1985, 1986, 1987, 1989, 1990, 1992, 1994, 1996, 1997, 1999, 2000, 2002, 2003, 2005, 2007, 2008, 2009, 2010, 2011, 2013, 2015, 2016, 2017, 2018, 2019, 2021, 2022, 2023, 2024, 2026, 2028, 2030, 2031, 2032, 2034, 2036, 2037, 2038, 2040, 2041, 2042, 2043, 2044, 2046, 2047, 2048, 2049, 2051, 2053, 2055, 2056, 2058, 2060, 2062, 2064, 2066, 2067, 2069, 2070, 2072, 2073, 2074, 2075, 2076, 2077, 2078, 2079, 2081, 2083, 2085, 2087, 2088, 2090, 2091, 2092, 2093, 2095, 2097, 2098, 2100, 2102, 2104, 2105, 2106, 2108, 2110, 2112, 2113, 2115, 2117, 2118, 2119, 2120, 2121, 2122, 2124, 2126, 2128, 2129, 2130, 2132, 2133, 2135, 2136, 2137, 2138, 2140, 2141, 2142, 2143, 2144, 2146, 2148, 2149, 2150, 2151, 2152, 2153, 2154, 2156, 2158, 2159, 2160, 2161, 2162, 2163, 2165, 2167, 2169, 2170, 2171, 2172, 2173, 2174, 2175, 2176, 2177, 2178, 2179, 2181, 2183, 2184, 2186, 2187, 2188, 2190, 2192, 2194, 2195, 2196, 2197, 2198, 2199, 2200, 2201, 2202, 2203, 2205, 2207, 2209, 2211, 2213, 2215, 2217, 2219, 2221, 2223, 2225, 2226, 2228, 2229, 2231, 2233, 2235, 2236, 2237, 2238, 2239, 2240, 2241, 2243, 2244, 2246, 2248, 2250, 2251, 2253, 2255, 2257, 2259, 2261, 2262, 2264, 2265, 2267, 2269, 2270, 2272, 2273, 2275, 2276, 2278, 2279, 2281, 2282, 2284, 2285, 2286, 2288, 2289, 2291, 2293, 2295, 2296, 2297, 2298, 2299, 2300, 2302, 2304, 2306, 2307, 2309, 2310, 2312, 2313, 2314, 2315, 2316, 2317, 2318, 2320, 2321, 2323, 2325, 2327, 2329, 2330, 2332, 2334, 2335, 2337, 2339, 2341, 2343, 2345, 2346, 2347, 2348, 2350, 2352, 2354, 2356, 2357, 2358, 2360, 2361, 2362, 2364, 2366, 2367, 2369, 2371, 2373, 2375, 2376, 2378, 2380, 2381, 2383, 2384, 2386, 2388, 2390, 2391, 2392, 2394, 2396, 2397, 2399, 2401, 2402, 2403, 2405, 2407, 2408, 2409, 2411, 2413, 2415, 2417, 2418, 2419, 2421, 2422, 2423, 2424, 2425, 2427, 2428, 2429, 2430, 2432, 2434, 2435, 2437, 2439, 2440, 2441, 2442, 2443, 2445, 2446, 2448, 2449, 2451, 2452, 2454, 2456, 2457, 2458, 2459, 2460, 2461, 2462, 2463, 2464, 2466, 2468, 2469, 2470, 2471, 2472, 2474, 2475, 2476, 2477, 2478, 2480, 2482, 2483, 2484, 2485, 2486, 2487, 2488, 2489, 2490, 2491, 2493, 2494, 2495, 2496, 2497, 2498, 2499, 2500, 2502, 2503, 2505, 2506, 2507, 2509, 2510, 2511, 2512, 2513, 2514, 2516, 2517, 2518, 2519, 2521, 2522, 2523, 2525, 2527, 2529, 2530, 2531, 2533, 2535, 2537, 2539, 2540, 2541, 2542, 2544, 2546, 2547, 2548, 2550, 2551, 2552, 2554, 2556, 2557, 2558, 2559, 2561, 2562, 2564, 2565, 2567, 2569, 2571, 2572, 2573, 2574, 2575, 2576, 2578, 2580, 2581, 2583, 2584, 2585, 2587, 2588, 2589, 2590, 2592, 2593, 2594, 2595, 2597, 2599, 2601, 2603, 2604, 2606, 2608, 2609, 2611, 2612, 2614, 2615, 2616, 2617, 2619, 2621, 2623, 2624, 2626, 2627, 2628, 2629, 2631, 2632, 2634, 2636, 2638, 2640, 2641, 2642, 2644, 2646, 2647, 2648, 2649, 2650, 2652, 2654, 2656, 2658, 2659, 2661, 2663, 2665, 2667, 2668, 2670, 2672, 2674, 2675, 2677, 2679, 2681, 2683, 2685, 2687, 2689, 2691, 2693, 2695, 2697, 2699, 2701, 2703, 2704, 2706, 2708, 2710, 2711, 2712, 2714, 2716, 2717, 2719, 2721, 2722, 2724, 2726, 2728, 2730, 2732, 2734, 2735, 2736, 2738, 2739, 2740, 2741, 2743, 2745, 2746, 2748, 2750, 2751, 2753, 2754, 2756, 2758, 2760, 2762, 2764, 2766, 2768, 2770, 2771, 2772, 2773, 2775, 2777, 2778, 2780, 2781, 2782, 2784, 2786, 2788, 2790, 2791, 2792, 2794, 2796, 2797, 2799, 2801, 2802, 2803, 2804, 2806, 2807, 2809, 2810, 2811, 2813, 2815, 2816, 2818, 2819, 2821, 2822, 2823, 2825, 2826, 2828, 2830, 2832, 2833, 2835, 2836, 2838, 2840, 2842, 2844, 2845, 2846, 2847, 2849, 2850, 2851, 2852, 2853, 2855, 2856, 2857, 2858, 2859, 2861, 2862, 2863, 2864, 2866, 2867, 2868, 2869, 2870, 2872, 2873, 2875, 2876, 2877, 2878, 2880, 2882, 2883, 2885, 2886, 2888, 2890, 2891, 2892, 2893, 2894, 2896, 2898, 2900, 2902, 2903, 2904, 2906, 2907, 2908, 2909, 2910, 2911, 2915, 2917, 2918, 2919, 2920, 2921, 2922, 2923, 2924, or 2926. Examples of nucleic acids encoding biomass-modulating polypeptides are set forth in SEQ ID NO: 1, 3, 7, 9, 11, 14, 16, 19, 21, 23, 28, 31, 33, 36, 40, 42, 44, 46, 48, 50, 52, 54, 58, 61, 66, 68, 72, 76, 78, 87, 89, 114, 116, 118, 125, 127, 130, 132, 134, 136, 138, 143, 145, 151, 153, 156, 159, 161, 164, 166, 168, 170, 172, 174, 177, 179, 181, 184, 186, 188, 190, 192, 202, 204, 206, 210, 212, 216, 221, 225, 227, 230, 241, 243, 253, 280, 283, 285, 290, 292, 296, 298, 301, 303, 305, 309, 312, 314, 321, 323, 325, 327, 329, 332, 334, 336, 339, 341, 344, 348, 352, 356, 359, 361, 366, 368, 376, 381, 388, 392, 394, 397, 399, 402, 405, 407, 413, 416, 418, 455, 458, 460, 463, 465, 471, 473, 476, 478, 481, 486, 496, 498, 500, 505, 508, 513, 519, 521, 523, 525, 527, 529, 541, 544, 549, 552, 556, 558, 561, 564, 567, 569, 571, 574, 578, 588, 594, 600, 603, 606, 608, 628, 630, 632, 639, 641, 643, 645, 647, 649, 652, 661, 668, 670, 673, 675, 678, 681, 683, 686, 688, 692, 695, 699, 701, 703, 708, 711, 713, 716, 724, 729, 736, 738, 740, 742, 748, 750, 754, 756, 758, 761, 763, 765, 767, 769, 771, 774, 777, 779, 781, 783, 785, 787, 790, 792, 797, 799, 803, 806, 808, 810, 812, 814, 816, 818, 821, 826, 829, 831, 833, 835, 837, 839, 841, 845, 847, 852, 854, 859, 862, 865, 867, 869, 872, 875, 877, 879, 881, 884, 886, 888, 891, 894, 898, 900, 904, 906, 911, 913, 918, 920, 922, 924, 927, 929, 931, 933, 938, 940, 942, 944, 946, 948, 951, 954, 956, 958, 961, 963, 966, 969, 971, 973, 975, 977, 979, 982, 984, 988, 990, 992, 994, 996, 999, 1001, 1004, 1006, 1010, 1012, 1015, 1017, 1019, 1021, 1028, 1030, 1032, 1035, 1037, 1040, 1047, 1053, 1055, 1057, 1059, 1063, 1065, 1068, 1073, 1075, 1080, 1082, 1084, 1086, 1089, 1091, 1094, 1098, 1100, 1103, 1106, 1109, 1116, 1118, 1123, 1125, 1127, 1129, 1131, 1134, 1136, 1138, 1140, 1143, 1145, 1147, 1149, 1151, 1153, 1155, 1159, 1161, 1163, 1165, 1167, 1170, 1172, 1174, 1178, 1182, 1184, 1186, 1189, 1191, 1195, 1197, 1199, 1202, 1204, 1206, 1208, 1211, 1213, 1215, 1217, 1220, 1223, 1225, 1227, 1229, 1231, 1233, 1237, 1239, 1242, 1244, 1246, 1248, 1250, 1254, 1256, 1258, 1260, 1262, 1264, 1267, 1269, 1271, 1273, 1275, 1277, 1280, 1282, 1284, 1286, 1288, 1290, 1292, 1295, 1298, 1301, 1303, 1305, 1307, 1311, 1315, 1317, 1319, 1321, 1323, 1327, 1331, 1335, 1337, 1346, 1348, 1351, 1354, 1356, 1362, 1365, 1368, 1374, 1378, 1383, 1385, 1387, 1390, 1392, 1394, 1396, 1398, 1400, 1404, 1406, 1408, 1411, 1413, 1415, 1417, 1419, 1423, 1425, 1427, 1430, 1432, 1434, 1436, 1438, 1442, 1444, 1447, 1449, 1451, 1453, 1455, 1458, 1460, 1462, 1464, 1466, 1468, 1470, 1472, 1474, 1476, 1478, 1480, 1484, 1487, 1491, 1494, 1496, 1499, 1501, 1504, 1510, 1513, 1517, 1519, 1521, 1527, 1530, 1532, 1536, 1539, 1541, 1543, 1545, 1548, 1551, 1553, 1556, 1559, 1561, 1566, 1568, 1570, 1573, 1576, 1579, 1582, 1585, 1587, 1590, 1592, 1597, 1601, 1606, 1608, 1610, 1614, 1617, 1620, 1623, 1625, 1627, 1631, 1633, 1635, 1638, 1641, 1643, 1645, 1651, 1653, 1655, 1659, 1663, 1665, 1667, 1673, 1675, 1678, 1685, 1687, 1690, 1692, 1694, 1696, 1698, 1703, 1709, 1716, 1718, 1720, 1722, 1726, 1728, 1730, 1733, 1736, 1738, 1740, 1742, 1744, 1748, 1750, 1752, 1754, 1756, 1760, 1763, 1768, 1771, 1775, 1778, 1780, 1782, 1785, 1787, 1789, 1791, 1793, 1796, 1800, 1802, 1814, 1818, 1820, 1824, 1830, 1832, 1836, 1838, 1840, 1843, 1846, 1848, 1851, 1860, 1862, 1864, 1868, 1870, 1873, 1875, 1878, 1880, 1884, 1886, 1888, 1890, 1892, 1896, 1899, 1901, 1905, 1907, 1909, 1911, 1914, 1916, 1921, 1923, 1925, 1927, 1930, 1932, 1934, 1937, 1939, 1941, 1943, 1945, 1947, 1950, 1952, 1954, 1956, 1958, 1960, 1962, 1964, 1966, 1968, 1970, 1977, 1981, 1984, 1988, 1991, 1993, 1995, 1998, 2001, 2004, 2006, 2012, 2014, 2020, 2025, 2027, 2029, 2033, 2035, 2039, 2045, 2050, 2052, 2054, 2057, 2059, 2061, 2063, 2065, 2068, 2071, 2080, 2082, 2084, 2086, 2089, 2094, 2096, 2099, 2101, 2103, 2107, 2109, 2111, 2114, 2116, 2123, 2125, 2127, 2131, 2134, 2139, 2145, 2147, 2155, 2157, 2164, 2166, 2168, 2180, 2182, 2185, 2189, 2191, 2193, 2204, 2206, 2208, 2210, 2212, 2214, 2216, 2218, 2220, 2222, 2224, 2227, 2230, 2232, 2234, 2242, 2245, 2247, 2249, 2252, 2254, 2256, 2258, 2260, 2263, 2266, 2268, 2271, 2274, 2277, 2280, 2283, 2287, 2290, 2292, 2294, 2301, 2303, 2305, 2308, 2311, 2319, 2322, 2324, 2326, 2328, 2331, 2333, 2336, 2338, 2340, 2342, 2344, 2349, 2351, 2353, 2355, 2359, 2363, 2365, 2368, 2370, 2372, 2374, 2377, 2379, 2382, 2385, 2387, 2389, 2393, 2395, 2398, 2400, 2404, 2406, 2410, 2412, 2414, 2416, 2420, 2426, 2431, 2433, 2436, 2438, 2444, 2447, 2450, 2453, 2455, 2465, 2467, 2473, 2479, 2481, 2492, 2501, 2504, 2508, 2515, 2520, 2524, 2526, 2528, 2532, 2534, 2536, 2538, 2543, 2545, 2549, 2553, 2555, 2560, 2563, 2566, 2568, 2570, 2577, 2579, 2582, 2586, 2591, 2596, 2598, 2600, 2602, 2605, 2607, 2610, 2613, 2618, 2620, 2622, 2625, 2630, 2633, 2635, 2637, 2639, 2643, 2645, 2651, 2653, 2655, 2657, 2660, 2662, 2664, 2666, 2669, 2671, 2673, 2676, 2678, 2680, 2682, 2684, 2686, 2688, 2690, 2692, 2694, 2696, 2698, 2700, 2702, 2705, 2707, 2709, 2713, 2715, 2718, 2720, 2723, 2725, 2727, 2729, 2731, 2733, 2737, 2742, 2744, 2747, 2749, 2752, 2755, 2757, 2759, 2761, 2763, 2765, 2767, 2769, 2774, 2776, 2779, 2783, 2785, 2787, 2789, 2793, 2795, 2798, 2800, 2805, 2808, 2812, 2814, 2817, 2820, 2824, 2827, 2829, 2831, 2834, 2837, 2839, 2841, 2843, 2848, 2854, 2860, 2865, 2871, 2874, 2879, 2881, 2884, 2887, 2889, 2895, 2897, 2899, 2901, 2905, 2912, 2914, 2916, or 2925, or in the Sequence Listing. The biomass-modulating polypeptide encoded by a recombinant nucleic acid can be a native biomass-modulating polypeptide, or can be heterologous to the cell. In some cases, the recombinant construct contains a nucleic acid that inhibits expression of a biomass-modulating polypeptide, operably linked to a regulatory region. Examples of suitable regulatory regions are described in the section entitled “Regulatory Regions.”

Vectors containing recombinant nucleic acid constructs such as those described herein also are provided. Suitable vector backbones include, for example, those routinely used in the art such as plasmids, viruses, artificial chromosomes, BACs, YACs, or PACs. Suitable expression vectors include, without limitation, plasmids and viral vectors derived from, for example, bacteriophage, baculoviruses, and retroviruses. Numerous vectors and expression systems are commercially available from such corporations as Novagen® (Madison, Wis.), Clontech® (Palo Alto, Calif.), Stratagene® (La Jolla, Calif.), and Invitrogen/Life Technologies® (Carlsbad, Calif.).

The vectors provided herein also can include, for example, origins of replication, scaffold attachment regions (SARs), and/or markers. A marker gene can confer a selectable phenotype on a plant cell. For example, a marker can confer biocide resistance, such as resistance to an antibiotic (e.g., kanamycin, G418, bleomycin, or hygromycin), or an herbicide (e.g., glyphosate, chlorsulfuron or phosphinothricin). In addition, an expression vector can include a tag sequence designed to facilitate manipulation or detection (e.g., purification or localization) of the expressed polypeptide. Tag sequences, such as luciferase, β-glucuronidase (GUS), green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

D. Regulatory Regions

The choice of regulatory regions to be included in a recombinant construct depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning regulatory regions relative to the coding sequence. Transcription of a nucleic acid can be modulated in a similar manner.

Some suitable regulatory regions initiate transcription only, or predominantly, in certain cell types. Methods for identifying and characterizing regulatory regions in plant genomic DNA are known, including, for example, those described in the following references: Jordano et al., Plant Cell, 1:855-866 (1989); Bustos et al., Plant Cell, 1:839-854 (1989); Green et al., EMBO J., 7:4035-4044 (1988); Meier et al., Plant Cell, 3:309-316 (1991); and Zhang et al., Plant Physiology, 110:1069-1079 (1996).

Examples of various classes of regulatory regions are described below. Some of the regulatory regions indicated below as well as additional regulatory regions are described in more detail in U.S. patent application Ser. Nos. 60/505,689; 60/518,075; 60/544,771; 60/558,869; 60/583,691; 60/619,181; 60/637,140; 60/757,544; 60/776,307; 10/957,569; 11/058,689; 11/172,703; 11/208,308; 11/274,890; 60/583,609; 60/612,891; 11/097,589; 11/233,726; 11/408,791; 11/414,142; 10/950,321; 11/360,017; PCT/US05/011105; PCT/US05/23639; PCT/US05/034308; PCT/US05/034343; and PCT/US06/038236; PCT/US06/040572; and PCT/US07/62762.

For example, the sequences of regulatory regions p326, YP0144, YP0190, p13879, YP0050, p32449, 21876, YP0158, YP0214, YP0380, PT0848, PT0633, YP0128, YP0275, PT0660, PT0683, PT0758, PT0613, PT0672, PT0688, PT0837, YP0092, PT0676, PT0708, YP0396, YP0007, YP0111, YP0103, YP0028, YP0121, YP0008, YP0039, YP0115, YP0119, YP0120, YP0374, YP0101, YP0102, YP0110, YP0117, YP0137, YP0285, YP0212, YP0097, YP0107, YP0088, YP0143, YP0156, PT0650, PT0695, PT0723, PT0838, PT0879, PT0740, PT0535, PT0668, PT0886, PT0585, YP0381, YP0337, PT0710, YP0356, YP0385, YP0384, YP0286, YP0377, PD1367, PT0863, PT0829, PT0665, PT0678, YP0086, YP0188, YP0263, PT0743 and YP0096 are set forth in the sequence listing of PCT/US06/040572; the sequence of regulatory region PT0625 is set forth in the sequence listing of PCT/US05/034343; the sequences of regulatory regions PT0623, YP0388, YP0087, YP0093, YP0108, YP0022 and YP0080 are set forth in the sequence listing of U.S. patent application Ser. No. 11/172,703; the sequence of regulatory region PR0924 is set forth in the sequence listing of PCT/US07/62762; and the sequences of regulatory regions p530c10, pOsFIE2-2, pOsMEA, pOsYp102, and pOsYp285 are set forth in the sequence listing of PCT/US06/038236.

It will be appreciated that a regulatory region may meet criteria for one classification based on its activity in one plant species, and yet meet criteria for a different classification based on its activity in another plant species.

i. Broadly Expressing Promoters

A promoter can be “broadly expressing” when it promotes transcription in all or most tissues, in more than one, but not necessarily in all, cell types within all tissues. For example, a broadly expressing promoter can promote transcription of an operably linked sequence in one or more of the shoot, shoot tip (apex), and leaves, but weakly or not at all in tissues such as roots or stems. As another example, a broadly expressing promoter can promote transcription of an operably linked sequence in one or more of the stem, shoot, shoot tip (apex), and leaves, but can promote transcription weakly or not at all in tissues such as reproductive tissues of flowers and developing seeds. Non-limiting examples of broadly expressing promoters that can be included in the nucleic acid constructs provided herein include the p326, YP0144, YP0190, p13879, YP0050, p32449, 21876, YP0158, YP0214, YP0380, PT0848, and PT0633 promoters. Additional examples include the cauliflower mosaic virus (CaMV) 35S promoter, the mannopine synthase (MAS) promoter, the 1′ or 2′ promoters derived from T-DNA of Agrobacterium tumefaciens, the figwort mosaic virus 34S promoter, actin promoters such as the rice actin promoter, and ubiquitin promoters such as the maize ubiquitin-1 promoter. In some cases, the CaMV 35S promoter is excluded from the category of broadly expressing promoters.

ii. Root Promoters

Root-active promoters confer transcription in root tissue, e.g., root endodermis, root epidermis, or root vascular tissues. In some embodiments, root-active promoters are root-preferential promoters, i.e., confer transcription only or predominantly in root tissue. Root-preferential promoters include the YP0128, YP0275, PT0625, PT0660, PT0683, and PT0758 promoters. Other root-preferential promoters include the PT0613, PT0672, PT0688, and PT0837 promoters, which drive transcription primarily in root tissue and to a lesser extent in ovules and/or seeds. Other examples of root-preferential promoters include the root-specific subdomains of the CaMV 35S promoter (Lam et al., Proc. Natl. Acad. Sci. USA, 86:7890-7894 (1989)), root cell specific promoters reported by Conkling et al., Plant Physiol., 93:1203-1211 (1990), and the tobacco RD2 promoter.

iii. Maturing Endosperm Promoters

In some embodiments, promoters that drive transcription in maturing endosperm can be useful. Transcription from a maturing endosperm promoter typically begins after fertilization and occurs primarily in endosperm tissue during seed development and is typically highest during the cellularization phase. Most suitable are promoters that are active predominantly in maturing endosperm, although promoters that are also active in other tissues can sometimes be used. Non-limiting examples of maturing endosperm promoters that can be included in the nucleic acid constructs provided herein include the napin promoter, the Arcelin-5 promoter, the phaseolin promoter (Bustos et al., Plant Cell, 1(9):839-853 (1989)), the soybean trypsin inhibitor promoter (Riggs et al., Plant Cell, 1(6):609-621 (1989)), the ACP promoter (Baerson et al., Plant Mol. Biol., 22(2):255-267 (1993)), the stearoyl-ACP desaturase promoter (Slocombe et al., Plant Physiol., 104(4):167-176 (1994)), the soybean α′ subunit of β-conglycinin promoter (Chen et al., Proc. Natl. Acad. Sci. USA, 83:8560-8564 (1986)), the oleosin promoter (Hong et al., Plant Mol. Biol., 34(3):549-555 (1997)), and zein promoters, such as the 15 kD zein promoter, the 16 kD zein promoter, 19 kD zein promoter, 22 kD zein promoter and 27 kD zein promoter. Also suitable are the Osgt-1 promoter from the rice glutelin-1 gene (Zheng et al., Mol. Cell. Biol., 13:5829-5842 (1993)), the beta-amylase promoter, and the barley hordein promoter. Other maturing endosperm promoters include the YP0092, PT0676, and PT0708 promoters.

iv. Ovary Tissue Promoters

Promoters that are active in ovary tissues such as the ovule wall and mesocarp can also be useful, e.g., a polygalacturonidase promoter, the banana TRX promoter, the melon actin promoter, YP0396, and PT0623. Examples of promoters that are active primarily in ovules include YP0007, YP0111, YP0092, YP0103, YP0028, YP0121, YP0008, YP0039, YP0115, YP0119, YP0120, and YP0374.

v. Embryo Sac/Early Endosperm Promoters

To achieve expression in embryo sac/early endosperm, regulatory regions can be used that are active in polar nuclei and/or the central cell, or in precursors to polar nuclei, but not in egg cells or precursors to egg cells. Most suitable are promoters that drive expression only or predominantly in polar nuclei or precursors thereto and/or the central cell. A pattern of transcription that extends from polar nuclei into early endosperm development can also be found with embryo sac/early endosperm-preferential promoters, although transcription typically decreases significantly in later endosperm development during and after the cellularization phase. Expression in the zygote or developing embryo typically is not present with embryo sac/early endosperm promoters.

Promoters that may be suitable include those derived from the following genes: Arabidopsis viviparous-1 (see, GenBank No. U93215); Arabidopsis atmycl (see, Urao, Plant Mol. Biol., 32:571-57 (1996); Conceicao, Plant, 5:493-505 (1994)); Arabidopsis FIE (GenBank No. AF129516); Arabidopsis MEA; Arabidopsis FIS2 (GenBank No. AF096096); and FIE 1.1 (U.S. Pat. No. 6,906,244). Other promoters that may be suitable include those derived from the following genes: maize MAC1 (see, Sheridan, Genetics, 142:1009-1020 (1996)); maize Cat3 (see, GenBank No. L05934; Abler, Plant Mol. Biol., 22:10131-1038 (1993)). Other promoters include the following Arabidopsis promoters: YP0039, YP0101, YP0102, YP0110, YP0117, YP0119, YP0137, DME, YP0285, and YP0212. Other promoters that may be useful include the following rice promoters: p530c10, pOsFIE2-2, pOsMEA, pOsYp102, and pOsYp285.

vi. Embryo Promoters

Regulatory regions that preferentially drive transcription in zygotic cells following fertilization can provide embryo-preferential expression. Most suitable are promoters that preferentially drive transcription in early stage embryos prior to the heart stage, but expression in late stage and maturing embryos is also suitable. Embryo-preferential promoters include the barley lipid transfer protein (Ltp1) promoter (Plant Cell Rep 20:647-654 (2001)), YP0097, YP0107, YP0088, YP0143, YP0156, PT0650, PT0695, PT0723, PT0838, PT0879, and PT0740.

vii. Photosynthetic Tissue Promoters

Promoters active in photosynthetic tissue confer transcription in green tissues such as leaves and stems. Most suitable are promoters that drive expression only or predominantly in such tissues. Examples of such promoters include the ribulose-1,5-bisphosphate carboxylase (RbcS) promoters such as the RbcS promoter from eastern larch (Larix laricina), the pine cab6 promoter (Yamamoto et al., Plant Cell Physiol., 35:773-778 (1994)), the Cab-1 promoter from wheat (Fejes et al., Plant Mol. Biol., 15:921-932 (1990)), the CAB-1 promoter from spinach (Lubberstedt et al., Plant Physiol., 104:997-1006 (1994)), the cab1R promoter from rice (Luan et al., Plant Cell, 4:971-981 (1992)), the pyruvate orthophosphate dikinase (PPDK) promoter from corn (Matsuoka et al., Proc. Natl. Acad. Sci. USA, 90:9586-9590 (1993)), the tobacco Lhcb1*2 promoter (Cerdan et al., Plant Mol. Biol., 33:245-255 (1997)), the Arabidopsis thaliana SUC2 sucrose-H+ symporter promoter (Truernit et al., Planta, 196:564-570 (1995)), and thylakoid membrane protein promoters from spinach (psaD, psaF, psaE, PC, FNR, atpC, atpD, cab, rbcS). Other photosynthetic tissue promoters include PT0535, PT0668, PT0886, YP0144, YP0380 and PT0585.

viii. Vascular Tissue Promoters

Examples of promoters that have high or preferential activity in vascular bundles include YP0087, YP0093, YP0108, YP0022, and YP0080. Other vascular tissue-preferential promoters include the glycine-rich cell wall protein GRP 1.8 promoter (Keller and Baumgartner, Plant Cell, 3(10):1051-1061 (1991)), the Commelina yellow mottle virus (CoYMV) promoter (Medberry et al., Plant Cell, 4(2):185-192 (1992)), and the rice tungro bacilliform virus (RTBV) promoter (Dai et al., Proc. Natl. Acad. Sci. USA, 101(2):687-692 (2004)).

ix. Inducible Promoters

Inducible promoters confer transcription in response to external stimuli such as chemical agents or environmental stimuli. For example, inducible promoters can confer transcription in response to hormones such as giberellic acid or ethylene, or in response to light or drought. Examples of drought-inducible promoters include YP0380, PT0848, YP0381, YP0337, PT0633, YP0374, PT0710, YP0356, YP0385, YP0396, YP0388, YP0384, PT0688, YP0286, YP0377, PD1367, and PD0901. Examples of nitrogen-inducible promoters include PT0863, PT0829, PT0665, and PT0886. Examples of shade-inducible promoters include PR0924 and PT0678. An example of a promoter induced by salt is rd29A (Kasuga et al. (1999) Nature Biotech 17: 287-291).

x. Basal Promoters

A basal promoter is the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

xi. Stem Promoters

A stem promoter may be specific to one or more stem tissues or specific to stem and other plant parts. Stem promoters may have high or preferential activity in, for example, epidermis and cortex, vascular cambium, procambium, or xylem. Examples of stem promoters include YP0018 which is disclosed in US20060015970 and CryIA(b) and CryIA(c) (Braga et al. 2003, Journal of New Seeds 5:209-221).

xii. Reproductive Tissue Promoters

Reproductive tissue promoters are regulatory sequences that drive expression primarily in, but are not necessarily exclusive to, tissues that are required for plant sexual reproduction. These tissues include, but are not limited to, inflorescence meristem, floral meristem, floral organs, and cells of the gametophyte. Examples of promoters that express in reproductive tissues include PD3720 in PCT/US2009/038792.

xiii. Other Promoters

Other classes of promoters include, but are not limited to, shoot-preferential, callus-preferential, trichome cell-preferential, guard cell-preferential such as PT0678, tuber-preferential, parenchyma cell-preferential, and senescence-preferential promoters. Promoters designated YP0086, YP0188, YP0263, PT0758, PT0743, PT0829, YP0119, and YP0096, as described in the above-referenced patent applications, may also be useful.

xiv. Other Regulatory Regions

A 5′ untranslated region (UTR) can be included in nucleic acid constructs described herein. A 5′ UTR is transcribed, but is not translated, and lies between the start site of the transcript and the translation initiation codon and may include the +1 nucleotide. A 3′ UTR can be positioned between the translation termination codon and the end of the transcript. UTRs can have particular functions such as increasing mRNA stability or attenuating translation. Examples of 3′ UTRs include, but are not limited to, polyadenylation signals and transcription termination sequences, e.g., a nopaline synthase termination sequence.

It will be understood that more than one regulatory region may be present in a recombinant polynucleotide, e.g., introns, enhancers, upstream activation regions, transcription terminators, and inducible elements. Thus, for example, more than one regulatory region can be operably linked to the sequence of a polynucleotide encoding a biomass-modulating polypeptide.

Regulatory regions, such as promoters for endogenous genes, can be obtained by chemical synthesis or by subcloning from a genomic DNA that includes such a regulatory region. A nucleic acid comprising such a regulatory region can also include flanking sequences that contain restriction enzyme sites that facilitate subsequent manipulation.

### IV. Transgenic Plants and Plant Cells

A. Transformation

The invention also features transgenic plant cells and plants comprising at least one recombinant nucleic acid construct described herein. A plant or plant cell can be transformed by having a construct integrated into its genome, i.e., can be stably transformed. Stably transformed cells typically retain the introduced nucleic acid with each cell division. A plant or plant cell can also be transiently transformed such that the construct is not integrated into its genome. Transiently transformed cells typically lose all or some portion of the introduced nucleic acid construct with each cell division such that the introduced nucleic acid cannot be detected in daughter cells after a sufficient number of cell divisions. Both transiently transformed and stably transformed transgenic plants and plant cells can be useful in the methods described herein.

Transgenic plant cells used in methods described herein can constitute part or all of a whole plant. Such plants can be grown in a manner suitable for the species under consideration, either in a growth chamber, a greenhouse, or in a field. Transgenic plants can be bred as desired for a particular purpose, e.g., to introduce a recombinant nucleic acid into other lines, to transfer a recombinant nucleic acid to other species, or for further selection of other desirable traits. Alternatively, transgenic plants can be propagated vegetatively for those species amenable to such techniques. As used herein, a transgenic plant also refers to progeny of an initial transgenic plant provided the progeny inherits the transgene. Seeds produced by a transgenic plant can be grown and then selfed (or outcrossed and selfed) to obtain seeds homozygous for the nucleic acid construct.

Transgenic plants can be grown in suspension culture, or tissue or organ culture. For the purposes of this invention, solid and/or liquid tissue culture techniques can be used. When using solid medium, transgenic plant cells can be placed directly onto the medium or can be placed onto a filter that is then placed in contact with the medium. When using liquid medium, transgenic plant cells can be placed onto a flotation device, e.g., a porous membrane that contacts the liquid medium. A solid medium can be, for example, Murashige and Skoog (MS) medium containing agar and a suitable concentration of an auxin, e.g., 2,4-dichlorophenoxyacetic acid (2,4-D), and a suitable concentration of a cytokinin, e.g., kinetin.

When transiently transformed plant cells are used, a reporter sequence encoding a reporter polypeptide having a reporter activity can be included in the transformation procedure and an assay for reporter activity or expression can be performed at a suitable time after transformation. A suitable time for conducting the assay typically is about 1-21 days after transformation, e.g., about 1-14 days, about 1-7 days, or about 1-3 days. The use of transient assays is particularly convenient for rapid analysis in different species, or to confirm expression of a heterologous biomass-modulating polypeptide whose expression has not previously been confirmed in particular recipient cells.

Techniques for introducing nucleic acids into monocotyledonous and dicotyledonous plants are known in the art, and include, without limitation, Agrobacterium-mediated transformation, viral vector-mediated transformation, electroporation and particle gun transformation, e.g., U.S. Pat. Nos. 5,538,880; 5,204,253; 6,329,571 and 6,013,863. If a cell or cultured tissue is used as the recipient tissue for transformation, plants can be regenerated from transformed cultures if desired, by techniques known to those skilled in the art.

B. Screening/Selection

A population of transgenic plants can be screened and/or selected for those members of the population that have a trait or phenotype conferred by expression of the transgene. For example, a population of progeny of a single transformation event can be screened for those plants having a desired level of expression of a biomass-modulating polypeptide or nucleic acid. Physical and biochemical methods can be used to identify expression levels. These include Southern analysis or PCR amplification for detection of a polynucleotide; Northern blots, S1 RNase protection, primer-extension, or RT-PCR amplification for detecting RNA transcripts; enzymatic assays for detecting enzyme or ribozyme activity of polypeptides and polynucleotides; and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are known. As an alternative, a population of plants comprising independent transformation events can be screened for those plants having a desired trait, such as a modulated level of biomass. Selection and/or screening can be carried out over one or more generations, and/or in more than one geographic location. In some cases, transgenic plants can be grown and selected under conditions which induce a desired phenotype or are otherwise necessary to produce a desired phenotype in a transgenic plant. In addition, selection and/or screening can be applied during a particular developmental stage in which the phenotype is expected to be exhibited by the plant. Selection and/or screening can be carried out to choose those transgenic plants having a statistically significant difference in a biomass level relative to a control plant that lacks the transgene. Selected or screened transgenic plants have an altered phenotype as compared to a corresponding control plant, as described in the “Transgenic Plant Phenotypes” section herein.

C. Plant Species

The polynucleotides and vectors described herein can be used to transform a number of monocotyledonous and dicotyledonous plants and plant cell systems, including species from one of the following families: Acanthaceae, Alliaceae, Alstroemeriaceae, Amaryllidaceae, Apocynaceae, Arecaceae, Asteraceae, Berberidaceae, Bixaceae, Brassicaceae, Bromeliaceae, Cannabaceae, Caryophyllaceae, Cephalotaxaceae, Chenopodiaceae, Colchicaceae, Cucurbitaceae, Dioscoreaceae, Ephedraceae, Erythroxylaceae, Euphorbiaceae, Fabaceae, Lamiaceae, Linaceae, Lycopodiaceae, Malvaceae, Melanthiaceae, Musaceae, Myrtaceae, Nyssaceae, Papaveraceae, Pinaceae, Plantaginaceae, Poaceae, Rosaceae, Rubiaceae, Salicaceae, Sapindaceae, Solanaceae, Taxaceae, Theaceae, or Vitaceae.

Suitable species may include members of the genus Abelmoschus, Abies, Acer, Agrostis, Allium, Alstroemeria, Ananas, Andrographis, Andropogon, Artemisia, Arundo, Atropa, Berberis, Beta, Bixa, Brassica, Calendula, Camellia, Camptotheca, Cannabis, Capsicum, Carthamus, Catharanthus, Cephalotaxus, Chrysanthemum, Cinchona, Citrullus, Coffea, Colchicum, Coleus, Cucumis, Cucurbita, Cynodon, Datura, Dianthus, Digitalis, Dioscorea, Elaeis, Ephedra, Erianthus, Erythroxylum, Eucalyptus, Festuca, Fragaria, Galanthus, Glycine, Gossypium, Helianthus, Hevea, Hordeum, Hyoscyamus, Jatropha, Lactuca, Linum, Lolium, Lupinus, Lycopersicon, Lycopodium, Manihot, Medicago, Mentha, Miscanthus, Musa, Nicotiana, Oryza, Panicum, Papaver, Parthenium, Pennisetum, Petunia, Phalaris, Phleum, Pinus, Poa, Poinsettia, Populus, Rauwolfia, Ricinus, Rosa, Saccharum, Salix, Sanguinaria, Scopolia, Secale, Solanum, Sorghum, Spartina, Spinacea, Tanacetum, Taxus, Theobroma, Triticosecale, Triticum, Uniola, Veratrum, Vinca, Vitis, and Zea.

Suitable species include Panicum spp., Sorghum spp., Miscanthus spp., Saccharum spp., Erianthus spp., Populus spp., Andropogon gerardii (big bluestem), Pennisetum purpureum (elephant grass), Phalaris arundinacea (reed canarygrass), Cynodon dactylon (bermudagrass), Festuca arundinacea (tall fescue), Spartina pectinata (prairie cord-grass), Medicago sativa (alfalfa), Arundo donax (giant reed), Secale cereale (rye), Salix spp. (willow), Eucalyptus spp. (eucalyptus), Triticosecale (triticum—wheat×rye) and bamboo.

Suitable species also include Helianthus annuus (sunflower), Carthamus tinctorius (safflower), Jatropha curcas (jatropha), Ricinus communis (castor), Elaeis guineensis (palm), Linum usitatissimum (flax), and Brassica juncea.

Suitable species also include Beta vulgaris (sugarbeet), and Manihot esculenta (cassava)

Suitable species also include Lycopersicon esculentum (tomato), Lactuca sativa (lettuce), Musa paradisiaca (banana), Solanum tuberosum (potato), Brassica oleracea (broccoli, cauliflower, Brussels sprouts), Camellia sinensis (tea), Fragaria ananassa (strawberry), Theobroma cacao (cocoa), Coffea arabica (coffee), Vitis vinifera (grape), Ananas comosus (pineapple), Capsicum annum (hot & sweet pepper), Allium cepa (onion), Cucumis melo (melon), Cucumis sativus (cucumber), Cucurbita maxima (squash), Cucurbita moschata (squash), Spinacea oleracea (spinach), Citrullus lanatus (watermelon), Abelmoschus esculentus (okra), and Solanum melongena (eggplant).

Suitable species also include Papaver somniferum (opium poppy), Papaver orientale, Taxus baccata, Taxus brevifolia, Artemisia annua, Cannabis sativa, Camptotheca acuminate, Catharanthus roseus, Vinca rosea, Cinchona officinalis, Colchicum autumnale, Veratrum californica, Digitalis lanata, Digitalis purpurea, Dioscorea spp., Andrographis paniculata, Atropa belladonna, Datura stomonium, Berberis spp., Cephalotaxus spp., Ephedra sinica, Ephedra spp., Erythroxylum coca, Galanthus wornorii, Scopolia spp., Lycopodium serratum (Huperzia serrata), Lycopodium spp., Rauwolfia serpentina, Rauwolfia spp., Sanguinaria canadensis, Hyoscyamus spp., Calendula officinalis, Chrysanthemum parthenium, Coleus forskohlii, and Tanacetum parthenium.

Suitable species also include Parthenium argentatum (guayule), Hevea spp. (rubber), Mentha spicata (mint), Mentha piperita (mint), Bixa orellana, and Alstroemeria spp.

Suitable species also include Rosa spp. (rose), Dianthus caryophyllus (carnation), Petunia spp. (petunia) and Poinsettia pulcherrima (poinsettia).

Suitable species also include Nicotiana tabacum (tobacco), Lupinus albus (lupin), Uniola paniculata (oats), bentgrass (Agrostis spp.), Populus tremuloides (aspen), Pinus spp. (pine), Abies spp. (fir), Acer spp. (maple), Hordeum vulgare (barley), Poa pratensis (bluegrass), Lolium spp. (ryegrass) and Phleum pratense (timothy).

In some embodiments, a suitable species can be a wild, weedy, or cultivated Pennisetum species such as, but not limited to, Pennisetum alopecuroides, Pennisetum arnhemicum, Pennisetum caffrum, Pennisetum clandestinum, Pennisetum divisum, Pennisetum glaucum, Pennisetum latifolium, Pennisetum macrostachyum, Pennisetum macrourum, Pennisetum orientale, Pennisetum pedicellatum, Pennisetum polystachion, Pennisetum polystachion ssp. Setosum, Pennisetum purpureum, Pennisetum setaceum, Pennisetum subangustum, Pennisetum typhoides, Pennisetum villosum, or hybrids thereof (e.g., Pennisetum purpureum×Pennisetum typhoidum).

In some embodiments, a suitable species can be a wild, weedy, or cultivated Miscanthus species and/or variety such as, but not limited to, Miscanthus×giganteus, Miscanthus sinensis, Miscanthus×ogiformis, Miscanthus floridulus, Miscanthus transmorrisonensis, Miscanthus oligostachyus, Miscanthus nepalensis, Miscanthus sacchariflorus, Miscanthus×giganteus ‘Amuri’, Miscanthus×giganteus ‘Wagara’, Miscanthus×giganteus ‘Illinois’, Miscanthus sinensis var. ‘Goliath’, Miscanthus sinensis var. ‘Roland’, Miscanthus sinensis var. ‘Africa’, Miscanthus sinensis var. ‘Fern Osten’, Miscanthus sinensis var. gracillimus, Miscanthus sinensis var. variegates, Miscanthus sinensis var. purpurascens, Miscanthus sinensis var. ‘Malepartus’, Miscanthus sacchariflorus var. ‘Robusta’, Miscanthus sinensis var. ‘Silberfedher’ (aka. Silver Feather), Miscanthus transmorrisonensis, Miscanthus condensatus, Miscanthus yakushimanum, Miscanthus var. ‘Alexander’, Miscanthus var. ‘Adagio’, Miscanthus var. ‘Autumn Light’, Miscanthus var. ‘Cabaret’, Miscanthus var. ‘Condensatus’, Miscanthus var. ‘Cosmopolitan’, Miscanthus var. ‘Dixieland’, Miscanthus var. ‘Gilded Tower’ (U.S. Patent No. PP14,743), Miscanthus var. ‘Gold Bar’ (U.S. Patent No. PP15,193), Miscanthus var. ‘Gracillimus’, Miscanthus var. ‘Graziella’, Miscanthus var. ‘Grosse Fontaine’, Miscanthus var. ‘Hinjo aka Little Nicky’™, Miscanthus var. ‘Juli’, Miscanthus var. ‘Kaskade’, Miscanthus var. ‘Kirk Alexander’, Miscanthus var. ‘Kleine Fontaine’, Miscanthus var. ‘Kleine Silberspinne’ (aka. ‘Little Silver Spider’), Miscanthus var. ‘Little Kitten’, Miscanthus var. ‘Little Zebra’ (U.S. Patent No. PP13,008), Miscanthus var. ‘Lottum’, Miscanthus var. ‘Malepartus’, Miscanthus var. ‘Morning Light’, Miscanthus var. ‘Mysterious Maiden’ (U.S. Patent No. PP16,176), Miscanthus var. ‘Nippon’, Miscanthus var. ‘November Sunset’, Miscanthus var. ‘Parachute’, Miscanthus var. ‘Positano’, Miscanthus var. ‘Puenktchen’(aka ‘Little Dot’), Miscanthus var. Rigoletto', Miscanthus var. ‘Sarabande’, Miscanthus var. ‘Silberpfeil’ (aka. Silver Arrow), Miscanthus var. ‘Silverstripe’, Miscanthus var. ‘Super Stripe’ (U.S. Patent No. PP18,161), Miscanthus var. ‘Strictus’, or Miscanthus var. ‘Zebrinus’.

In some embodiments, a suitable species can be a wild, weedy, or cultivated sorghum species and/or variety such as, but not limited to, Sorghum almum, Sorghum amplum, Sorghum angustum, Sorghum arundinaceum, Sorghum bicolor (such as bicolor, guinea, caudatumi, kafir, and durra), Sorghum brachypodum, Sorghum bulbosum, Sorghum burmahicum, Sorghum controversum, Sorghum drummondii, Sorghum ecarinatum, Sorghum exstans, Sorghum grande, Sorghum halepense, Sorghum interjectum, Sorghum intrans, Sorghum laxiflorum, Sorghum leiocladum, Sorghum macrospermum, Sorghum matarankense, Sorghum miliaceum, Sorghum nigrum, Sorghum nitidum, Sorghum plumosum, Sorghum propinquum, Sorghum purpureosericeum, Sorghum stipoideum, Sorghum sudanensese, Sorghum timorense, Sorghum trichocladum, Sorghum versicolor, Sorghum virgatum, Sorghum vulgare, or hybrids such as Sorghum×almum, Sorghum×sudangrass or Sorghum×drummondii.

Thus, the methods and compositions can be used over a broad range of plant species, including species from the dicot genera Brassica, Carthamus, Glycine, Gossypium, Helianthus, Jatropha, Parthenium, Populus, and Ricinus; and the monocot genera Elaeis, Festuca, Hordeum, Lolium, Oryza, Panicum, Pennisetum, Phleum, Poa, Saccharum, Secale, Sorghum, Triticosecale, Triticum, and Zea. In some embodiments, a plant is a member of the species Panicum virgatum (switchgrass), Sorghum bicolor (sorghum, sudangrass), Miscanthus giganteus (miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza sativa (rice), Helianthus annuus (sunflower), Medicago sativa (alfalfa), Beta vulgaris (sugarbeet), or Pennisetum glaucum (pearl millet).

In certain embodiments, the polynucleotides and vectors described herein can be used to transform a number of monocotyledonous and dicotyledonous plants and plant cell systems, wherein such plants are hybrids of different species or varieties of a specific species (e.g., Saccharum sp.×Miscanthus sp., Sorghum sp.×Miscanthus sp., e.g., Panicum virgatum×Panicum amarum, Panicum virgatum×Panicum amarulum, and Pennisetum purpureum×Pennisetum typhoidum).

D. Transgenic Plant Phenotypes

In some embodiments, a plant in which expression of a biomass-modulating polypeptide is modulated can have increased levels of biomass in plants. For example, a biomass-modulating polypeptide described herein can be expressed in a transgenic plant, resulting in increased levels of vegetative tissue. The biomass level can be increased by at least 2 percent, e.g., 2, 3, 4, 5, 6, 7, 8, 9, 10, 11, 12, 13, 14, 15, 16, 17, 18, 19, 20, 25, 30, 35, 40, 45, 50, 55, 60, or more than 60 percent, as compared to the biomass level in a corresponding control plant that does not express the transgene. In some embodiments, a plant in which expression of a biomass-modulating polypeptide is modulated can have decreased levels of seed production. The level can be decreased by at least 2 percent, e.g., 2, 3, 4, 5, 10, 15, 20, 25, 30, 35, or more than 35 percent, as compared to the seed production level in a corresponding control plant that does not express the transgene. In some cases, an increase in the level of biomass is expressed as a fold change, e.g., the level of lignin in a transgenic plant described herein can be from about 0.20 fold to about 10-fold, about 0.50-fold to about 5-fold, or about 1-fold to about 2.3-fold higher than the level of biomass in corresponding control plants.

Increases in seed production in such plants can provide improved nutritional availability in geographic locales where intake of plant foods is often insufficient, or for biofuel production. In some embodiments, decreases in biomass in such plants can be useful in situations where vegetative tissues are not the primary plant part that is harvested for human or animal consumption (i.e., seeds are harvested).

In some embodiments, a plant in which expression of a biomass-modulating polypeptide is modulated can have increased or decreased levels of biomass in one or more plant tissues, e.g., vegetative tissues, reproductive tissues, or root tissues. For example, the biomass level can be increased by at least 2 percent, e.g., 2, 3, 4, 5, 6, 7, 8, 9, 10, 11, 12, 13, 14, 15, 16, 17, 18, 19, 20, 25, 30, 35, 40, 45, 50, 55, 60, or more than 60 percent, as compared to the biomass level in a corresponding control plant that does not express the transgene. In some embodiments, a plant in which expression of a biomass-modulating polypeptide is modulated can have decreased levels of biomass in one or more plant tissues. The biomass level can be decreased by at least 2 percent, e.g., 2, 3, 4, 5, 10, 15, 20, 25, 30, 35, or more than 35 percent, as compared to the biomass level in a corresponding control plant that does not express the transgene.

Increases in biomass in such plants can provide improved food quantity, or improved energy production. Decreases in biomass can provide more efficient partitioning of nutrients to plant part(s) that are harvested for human or animal consumption.

Typically, a difference in the amount of biomass or biomass component in a transgenic plant or cell relative to a control plant or cell is considered statistically significant at p≦0.05 with an appropriate parametric or non-parametric statistic, e.g., Chi-square test, Student's t-test, Mann-Whitney test, or F-test. In some embodiments, a difference in the amount of biomass is statistically significant at p<0.01, p<0.005, or p<0.001. A statistically significant difference in, for example, the amount of biomass in a transgenic plant compared to the amount of a control plant indicates that the recombinant nucleic acid present in the transgenic plant results in altered biomass levels or altered content of a biomass component.

The phenotype of a transgenic plant is evaluated relative to a control plant. A plant is said “not to express” a polypeptide when the plant exhibits less than 10%, e.g., less than 9%, 8%, 7%, 6%, 5%, 4%, 3%, 2%, 1%, 0.5%, 0.1%, 0.01%, or 0.001%, of the amount of polypeptide or mRNA encoding the polypeptide exhibited by the plant of interest. Expression can be evaluated using methods including, for example, RT-PCR, Northern blots, S1 RNase protection, primer extensions, Western blots, protein gel electrophoresis, immunoprecipitation, enzyme-linked immunoassays, chip assays, and mass spectrometry. It should be noted that if a polypeptide is expressed under the control of a tissue-preferential or broadly expressing promoter, expression can be evaluated in the entire plant or in a selected tissue. Similarly, if a polypeptide is expressed at a particular time, e.g., at a particular time in development or upon induction, expression can be evaluated selectively at a desired time period.

Biomass can include harvestable plant tissues such as leaves, stems, and reproductive structures, or all plant tissues such as leaves, stems, roots, and reproductive structures. In some embodiments, biomass encompasses only above ground plant parts. In some embodiments, biomass encompasses only stem plant parts. In some embodiments, biomass encompasses only above ground plant parts except inflorescence and seed parts of a plant. Biomass can be measured as described in the examples section. Biomass can be quantified as dry matter yield, which is the mass of biomass produced (usually reported in T/acre) if the contribution of water is subtracted from the fresh mater weight. Dry matter yield (DMY) yield is calculated using the fresh matter weight (FMW) and a measurement of weight percent moisture (M) in the following equation. DMY=((100−M)/100)*FMW. Biomass can be quantified as fresh matter yield, which is the mass of biomass produced (usually reported in T/acre) on an as-received basis, which includes the weight of moisture.

In some embodiments, a plant in which expression of a biomass-modulating polypeptide is modulated can have increased or decreased lignin content in one or more plant tissues, e.g., vegetative tissues, reproductive tissues, or root tissues. The lignin content can be increased by at least 2 percent, e.g., 2, 3, 4, 5, 6, 7, 8, 9, 10, 11, 12, 13, 14, 15, 16, 17, 18, 19, 20, 25, 30, 35, 40, 45, 50, 55, 60, or more than 60 percent, as compared to the lignin content in a corresponding control plant that does not express the transgene. In some embodiments, a transgenic plant can have a 5 to 100 percent, e.g., 13 to 50 percent, 11 to 100 percent, 10 to 35 percent, 5 to 25 percent, or 28 to 30 percent increase in lignin, as compared to the lignin content of a transgenic plant that does not express the transgene.

In some embodiments, a plant in which expression of a biomass-modulating polypeptide is modulated can have increased or decreased lignin content in one or more plant tissues, e.g., vegetative tissues, reproductive tissues, or root tissues. The lignin content can be increased by at least 2 percent, e.g., 2, 3, 4, 5, 6, 7, 8, 9, 10, 11, 12, 13, 14, 15, 16, 17, 18, 19, 20, 25, 30, 35, 40, 45, 50, 55, 60, or more than 60 percent, as compared to the lignin content in a corresponding control plant that does not express the transgene. In some embodiments, a transgenic plant can have a 5 to 100 percent, e.g., 13 to 50 percent, 11 to 100 percent, 10 to 35 percent, 5 to 25 percent, or 28 to 30 percent increase in lignin, as compared to the lignin content of a transgenic plant that does not express the transgene.

In some embodiments, a plant in which expression of a biomass-modulating polypeptide is modulated can have decreased lignin content. The lignin content can be decreased by at least 2 percent, e.g., 2, 3, 4, 5, 10, 15, 20, 25, 30, 35, or more than 35 percent, as compared to the seed production level in a corresponding control plant that does not express the transgene.

Provided herein also are methods for modulating (e.g., altering, increasing, or decreasing) the lignin content of a plant. Such a method can include growing a plant cell that includes a nucleic acid encoding a biomass-modulating polypeptide as described herein. In some cases, the nucleic acid can be a nucleotide sequence having 80% sequence identity to a nucleotide sequence set forth in SEQ ID NOs: 114, 283, 458, 628, 668, 692, 716, 845, 1053, 1106, 1271, 1480, 1568, 1623, 1726, 1796, 2189, 2620, or 2879. A plant cell can be from a plant capable of producing lignin.

A transgenic plant, tissue, or cell comprising a biomass-modulating sequence can have a modulated amount and/or rate of lignin biosynthesis. Plants, tissues, or cells containing a nucleic acid construct described herein typically have a difference in the amount and/or rate of synthesis of lignin, relative to a corresponding control plant, tissue, or cell that is not transformed with the nucleic acid construct.

A number of different types of lignin, based on chemical and structural features, can be produced by different species of plants, by different tissues of the same plant, or by different parts of the same plant cell. Such lignins include, without limitation, lignins comprising primarily or only coniferyl alcohols such as guaiacyl lignin, lignins comprising primarily or only sinapyl alcohols such as syringyl lignin, lignins comprising primarily or only p-coumaryl alcohols such as p-hydroxyphenyl lignin, and lignins comprising primarily or only coniferyl and sinapyl alcohols such as guaiacyl-syringyl lignin. In some cases, a transgenic plant can exhibit a difference in the ratio of syringyl lignin to guaiacyl lignin (S:G ratio). Plants with higher G content exhibit more highly condensed lignin with a preponderance of more stable, but also more recalcitrant bonds. Generally, S subunits in lignin can be chemically degraded more easily than linkages between G subunits. A transgenic plant described herein can exhibit an increase in the S:G ratio of at least about 4 percent, e.g., more than 3, 4, 5, 6, 7, 8, 9, 10, 20, 30, 40, 50, or 60 percent. In some cases, the S:G ratio of a transgenic plant is decreased relative to the S:G ratio of a wild-type control plant. For example, the S:G ratio can be decreased by at least 0.3 percent, e.g., more than 0.2, 0.3, 0.4, 0.5, 1, 5, 10, or 20 percent.

In addition, other compounds can be incorporated into lignins, including, without limitation, coniferyl/sinapyl p-coumarate, coniferyl/sinapyl p-hydroxybenzoate, coniferyl/sinapyl acetate, ferulate esters, 5-hydroxy-coniferyl alcohol, 3,4-dihydroxy-cinnamyl alcohol, feruloyl amides such as tyramine ferulate, coniferaldehyde/sinapaldehyde, vanillin/syringaldehyde, benzodioxanes, 5-hydroxyguaiacyl, and dihydroconiferyl/dihydro-p-coumaryl alcohol.

The amount and/or rate of synthesis of a type of lignin can be modulated, e.g., increased or decreased, in a transgenic plant, tissue, or cell relative to a control plant, tissue, or cell using the methods described herein. In some cases, the amounts of two or more types of lignin (e.g., two, three, four, five, six, seven, eight, nine, ten or even more types of lignin) can be independently modulated relative to a control plant, tissue, or cell.

In some embodiments, the amount of lignin is decreased in transgenic plants, tissues, or cells described herein (e.g., transgenic plants expressing a biomass-modulating polypeptide as described herein). A decrease ratio can be expressed as the ratio of the lignin in such a transgenic plant, tissue, or cell on a weight basis (e.g., fresh weight basis) as compared to the lignin in a corresponding control plant, tissue, or cell (e.g., a corresponding plant, tissue, or cell that lacks the nucleic acid encoding the biomass-modulating polypeptide). The decrease ratio can be from about 0.05 to about 0.90. In certain cases, the ratio can be from about 0.2 to about 0.6, or from about 0.4 to about 0.6, or from about 0.3 to about 0.5, or from about 0.2 to about 0.4.

In some cases, a decrease in the amount of lignin in a transgenic plant described herein can be calculated as a percent decrease in the weight of lignin extracted per weight of tissue of the transgenic plant relative to the weight of lignin extracted per weight of tissue of a corresponding control plant. For example, the amount of lignin in a tissue of a transgenic plant provided herein can be decreased by about 5% to about 100% relative to the amount of lignin in a tissue of a corresponding control plant.

In some embodiments, the amount of lignin in a transgenic plant as described herein can be decreased by about 0.45% to about 100%, or about 10% to about 70%, or about 20% to about 68%, or about 50% to about 66%, or about 53% to about 66%, or about 55% to about 65%, or about 57% to about 71%, or about 50% to about 70%, or about 55% to about 60%, or about 60% to about 65% relative to the amount of lignin in corresponding control plants.

The decrease in the amount of lignin can be restricted in some embodiments to particular tissues and/or organs, relative to other tissues and/or organs. For example, a transgenic plant can have a decreased amount of lignin in stem tissue relative to leaf tissue. The decreased amount of lignin can be in the cell wall of plant cells such as tracheids, xylem fibres, and sclereids.

An increase in the amount of lignin in a transgenic plant, tissue, or cell described herein can be from about 1.02-fold to about 10-fold, about 1.03-fold to about 1.7-fold, or about 1.04-fold to about 1.6-fold, or about 1.05-fold to about 1.7-fold, or about 1.06-fold to about 2.3-fold, or about 1.07-fold to about 2.5-fold, or about 1.08-fold to about 2-fold, or about 1.09-fold to about 2.4-fold, or about 1.1-fold to about 2-fold, or about 1.2-fold to about 3-fold, or about 1.3-fold to about 2.5-fold, or about 1.4-fold to about 3-fold, or about 1.5-fold to about 5-fold, or about 2-fold to about 6-fold, or about 2-fold to about 5-fold, or about 1.5-fold to 7-fold, or about 3-fold to about 4-fold, or about 3-fold to about 7.5-fold, or about 4-fold to about 8-fold, or about 5-fold to about 10-fold higher than the amount in corresponding control plants, tissues, or cells.

In some cases, an increase in the amount of lignin in a transgenic plant described herein can be calculated as a percent increase in the amount of lignin detected per weight of tissue of the transgenic plant relative to the weight of lignin extracted per weight of tissue of a corresponding control plant. For example, the amount of lignin in a tissue of a transgenic plant provided herein can be increased by about 0.10% to about 10%, or about 5% to about 15%, or about 20% to about 50%, or about 25% to about 40%, or about 30% to about 60%, or about 50% to about 75%, or about 75% to about 100%, or about 90% to about 150%, or about 50% to about 200%, or about 100% to about 300%, or about 150% to about 500%, or about 200 to about 600%, or about 300% to about 800% relative to the amount of lignin in a tissue of a corresponding control plant.

In some embodiments, the lignin that is increased in a tissue of a transgenic plant described herein is either not produced by or is not detectable in a corresponding tissue of a control plant. Thus, in such embodiments, the increase in lignin is infinitely high. For example, in certain cases, a biomass-modulating polypeptide described herein may activate a biosynthetic pathway in a plant tissue that is not normally activated or operational in a control plant tissue and one or more new types of lignin that were not previously produced in that plant tissue can be produced.

The increase in amount of lignin can be restricted in some embodiments to particular tissues and/or organs, relative to other tissues and/or organs. For example, a transgenic plant can have an increased amount of lignin in stem tissue relative to leaf tissue. The increased amount of lignin can be in the cell wall of plant cells such as tracheids, xylem fibers, and sclereids.

The amount of lignin in a plant can be determined by known techniques, e.g., the acid detergent, Klason, acetyl bromide, and permanganate lignin methods. See, for example, Hatfield and Fukushima, Crop Sci., 45:832-839 (2005); and Methods in Lignin Chemistry, Dence and Lin, eds., Springer-Verlag, Berlin, p. 33-61 (1992). Pyrolysis-gas chromatography-mass spectrometry, liquid chromatography-mass spectrometry (LC-MS), or a degradative method, e.g., the DFRC method or thioacidolysis, combined with mass spectrometry also can be used. If desired, the composition and structure of lignin can be characterized by GC-MS, LC-MS, nuclear magnetic resonance spectroscopy, Fourier-transform infrared spectroscopy, and/or other known techniques. In addition, histochemical analysis can be performed to determine the amount and distribution of lignin in a plant. For example, tissue sections can be stained with toluidine blue O (TBO), the Wiesner reagent, or the Mäule reagent. TBO is a metachromatic stain that imparts a turquoise color to lignified cell walls and stains non-lignified cell walls purple. Phloroglucinol stains lignified cells red upon reaction with hydroxycinnamaldehyde groups present in the polymer. The Mäule reagent is a histochemical stain that allows syringyl lignin to be distinguished chromogenically from guaiacyl lignin in situ. A pink or red color can indicate the presence of syringyl units, whereas a light to dark brown

Also provided are methods for modulating the glucan content of a plant. Such a method can include growing a plant cell that includes a nucleic acid encoding a biomass-modulating polypeptide as described herein. In some cases, a biomass-modulating peptide is selected from the group consisting of SEQ ID NOs: 668, 1053, 1116, 1271, 1426, 2653, or 2707, and functional homologs thereof. The glucan content of a transgenic plant can be decreased relative to a control plant. An decrease in the amount of glucan in a transgenic plant, tissue, or cell described herein can be from about 7 percent to about 20 percent, e.g., from more than 6, 7, 8, 9, 10, 11, 12, 13, 14, 15, 16, 17, 18, 19 or 20 percent, as compared to the amount of glucan present in a corresponding control plant, tissue or cell. In some cases, the glucan content of a transgenic plant can be increased relative to a control plant. An increase in the amount of glucan in a transgenic plant, tissue, or cell described herein can be from about 0.40 percent to about 55 percent, e.g., more than 0.35, 0.40, 0.50, 0.75, 1, 5, 10, 15, 20, 25, 30, 35, 40, 45, 50, or 55 percent, as compared to the amount of glucan present in a corresponding control plant, tissue or cell. Glucan content can be determined directly or indirectly. For example, a direct measurement of glucan content can be performed using ASTM methods E1758-01 Determination of Biomass Sugars by High Performance Liquid Chromatography. Glucan content can be predicted using an appropriate NIR model. See, e.g., WO 2009-059176.

Also provided are methods for decreasing the ash content of a plant. Such a method can include growing a plant cell that includes a nucleic acid encoding a biomass-modulating polypeptide as described herein. Ash content can be decreased by about 4 percent to about 25 percent, e.g., more than 4, 5, 8, 10, 12, 15, 18, or 23 percent. In some embodiments, a biomass modulating sequence is in an antisense orientation relative to the regulatory region. In some cases, a biomass-modulating sequence is transcribed into an interfering RNA. For example, the nucleotide sequence set forth in SEQ ID NO:2914 (corresponding to nucleic acid residues 22 to 325 of SEQ ID NO:877) or SEQ ID NO:2925 (corresponding to the DNA complement of nucleic acid residues 23 to 412 of SEQ ID NO:2916) comprise a suitable RNAi construct. Ash content can be determined using, for example, ASTM Method E1755-01 or a NIR composition model. See, e.g., WO 2009-059176. The ash content for samples that are selected using the NIR model can be confirmed directly using ASTM Method E1755-01. Typically, the amount of moisture present in the biomass sample is determined first using an appropriate moisture analyzer, e.g., Denver IR120 Moisture Analyzer. See ASTM E1756-08 “Standard Test Method for Determination of Total Solids in Biomass” ASTM International, West Conshohocken, Pa., DOI: 10.1520/E1755-01R07 be used to determine moisture content.

### V. Modifying Endogenous Nucleic Acids Encoding Biomass-Modulating Polypeptides

This document also features plant cells and plants in which an endogenous biomass-modulating nucleic acid described herein has been modified (e.g., a regulatory region, intron, or coding region of the biomass-modulating nucleic acid has been modified). The biomass of such plants is altered relative to the corresponding level of a control plant in which the endogenous nucleic acid is not modified. Such plants are referred to herein as modified plants and may be used to produce, for example, increased amounts of biomass.

Endogenous nucleic acid can be modified by homologous recombination techniques. For example, sequence specific endonucleases (e.g., zinc finger nucleases (ZFNs)) and meganucleases can be used to stimulate homologous recombination at endogenous plant genes. See, e.g., Townsend et al., Nature 459:442-445 (2009); Tovkach et al., Plant J., 57:747-757 (2009); and Lloyd et al., Proc. Natl. Acad. Sci. USA, 102:2232-2237 (2005). In particular, ZFNs engineered to create DNA double strand breaks at specific loci can be used to make targeted sequence changes in endogenous plant genes. For example, an endogenous plant gene can be replaced with a variant containing one or more mutations (e.g., produced using site-directed mutagenesis or directed evolution).

In some embodiments, endogenous nucleic acids can be modified by methylation or demethylation such that the expression of the modified endogenous nucleic acid is altered. For example, a double stranded RNA can be used to activate gene expression by targeting noncoding regulatory regions in gene promoters. See Shibuya et al., Proc Natl Acad Sci USA, 106(5): 1660-1665 (2009); and Li et al., Proc Natl Acad Sci USA, 103(46):17337-42 (2006).

In some embodiments, endogenous nucleic acids can be modified using activation tagging. For example, a vector containing multiple copies of an enhancer element from the constitutively active promoter of the cauliflower mosaic virus (CaMV) 35S gene can be used to activate an endogenous gene. See, Weigel et al., Plant Physiology, 122:1003-1013 (2000).

In some embodiments, endogenous nucleic acids can be modified by introducing an engineered transcription activation/repression factor (e.g., zinc finger protein transcription factor, or ZFP TF). See, for example, the world wide web at sangamo.com/tech/tech_plat_over.html#whatarezfp. An engineered transcription activation/repression factor (such as ZFP TF) can activate, repress, or switch the target endogenous biomass gene expression by binding specifically to the promoter region or coding region of the endogenous gene.

In some embodiments, endogenous nucleic acids can be modified by mutagenesis. Genetic mutations can be introduced within regenerable plant tissue using one or more mutagenic agents. Suitable mutagenic agents include, for example, ethyl methane sulfonate (EMS), N-nitroso-N-ethylurea (ENU), methyl N-nitrosoguanidine (MNNG), ethidium bromide, diepoxybutane, ionizing radiation, x-rays, UV rays and other mutagens known in the art. Suitable types of mutations include, for example, insertions or deletions of nucleotides, and transitions or transversions in the endogenous nucleic acid sequence. In one embodiment, TILLING (Targeted Induced Local Lesions In Genomes) can be used to produce plants having a modified endogenous nucleic acid. TILLING combines high-density mutagenesis with high-throughput screening methods. See, for example, McCallum et al., Nat Biotechnol 18: 455-457 (2000); reviewed by Stemple, Nat Rev Genet. 5(2):145-50 (2004).

In some embodiments, an endogenous nucleic acid can be modified via a gene silencing technique. See, for example, the section herein regarding “Inhibition of Expression of a Biomass-Modulating Polypeptide.”

A population of plants can be screened and/or selected for those members of the population that have a modified nucleic acid. A population of plants also can be screened and/or selected for those members of the population that have a trait or phenotype conferred by expression of the modified nucleic acid. As an alternative, a population of plants can be screened for those plants having a desired trait, such as a modulated level of biomass. For example, a population of progeny can be screened for those plants having a desired level of expression of a biomass-modulating polypeptide or nucleic acid. Physical and biochemical methods can be used to identify modified nucleic acids and/or expression levels as described with transgenic plants. Selection and/or screening can be carried out over one or more generations, and/or in more than one geographic location. In some cases, plants can be grown and selected under conditions which induce a desired phenotype or are otherwise necessary to produce a desired phenotype in a modified plant. In addition, selection and/or screening can be applied during a particular developmental stage in which the phenotype is expected to be exhibited by the plant. Selection and/or screening can be carried out to choose those modified plants having a statistically significant difference in a biomass level relative to a control plant in which the nucleic acid has not been modified. Selected or screened modified plants have an altered phenotype as compared to a corresponding control plant, as described in the “Transgenic Plant Phenotypes” section herein.

Although a plant or plant cell in which an endogenous biomass-modulating nucleic acid has been modified is not transgenic for that particular nucleic acid, it will be appreciated that such a plant or cell may contain transgenes. For example, a modified plant can contain a transgene for other traits, such as herbicide tolerance or insect resistance. As another example, a modified plant can contain one or more transgenes that, in conjunction with modifications of one or more endogenous nucleic acids, exhibits an increase in biomass.

As with transgenic plant cells, modified plant cells can constitute part or all of a whole plant. Such plants can be grown in the same manner as described for transgenic plants and can be bred or propagated in the same manner as described for transgenic plants.

### VI. Plant Breeding

Genetic polymorphisms that are useful in such methods include simple sequence repeats (SSRs, or microsatellites), rapid amplification of polymorphic DNA (RAPDs), single nucleotide polymorphisms (SNPs), amplified fragment length polymorphisms (AFLPs) and restriction fragment length polymorphisms (RFLPs). SSR polymorphisms can be identified, for example, by making sequence specific probes and amplifying template DNA from individuals in the population of interest by PCR. For example, PCR techniques can be used to enzymatically amplify a genetic marker associated with a nucleotide sequence conferring a specific trait (e.g., nucleotide sequences described herein). PCR can be used to amplify specific sequences from DNA as well as RNA, including sequences from total genomic DNA or total cellular RNA. When using RNA as a source of template, reverse transcriptase can be used to synthesize complementary DNA (cDNA) strands. Various PCR methods are described, for example, in PCR Primer: A Laboratory Manual, Dieffenbach and Dveksler, eds., Cold Spring Harbor Laboratory Press, 1995.

Generally, sequence information from polynucleotides flanking the region of interest or beyond is employed to design oligonucleotide primers that are identical or similar in sequence to opposite strands of the template to be amplified. Primers are typically 14 to 40 nucleotides in length, but can range from 10 nucleotides to hundreds of nucleotides in length. Template and amplified DNA is repeatedly denatured at a high temperature to separate the double strand, then cooled to allow annealing of primers and the extension of nucleotide sequences through the microsatellite, resulting in sufficient DNA for detection of PCR products. If the probes flank an SSR in the population, PCR products of different sizes will be produced. See, e.g., U.S. Pat. No. 5,766,847.

PCR products can be qualitative or quantitatively analyzed using several techniques. For example, PCR products can be stained with a fluorescent molecule (e.g., PicoGreen® or OliGreen®) and detected in solution using spectrophotometry or capillary electrophoresis. In some cases, PCR products can be separated in a gel matrix (e.g., agarose or polyacrylamide) by electrophoresis, and size-fractionated bands comprising PCR products can be visualized using nucleic acid stains. Suitable stains can fluoresce under UV light (e.g., Ethidium bromide, GR Safe, SYBR® Green, or SYBR® Gold). The results can be visualized via transillumination or epi-illumination, and an image of the fluorescent pattern can be acquired using a camera or scanner, for example. The image can be processed and analyzed using specialized software (e.g., ImageJ) to measure and compare the intensity of a band of interest against a standard loaded on the same gel.

Alternatively, SSR polymorphisms can be identified by using PCR product(s) as a probe against Southern blots from different individuals in the population. See, U. H. Refseth et al., (1997) Electrophoresis 18: 1519. Briefly, PCR products are separated by length through gel electrophoresis and transferred to a membrane. SSR-specific DNA probes, such as oligonucleotides labeled with radioactive, fluorescent, or chromogenic molecules, are applied to the membrane and hybridize to bound PCR products with a complementary nucleotide sequence. The pattern of hybridization can be visualized by autoradiography or by development of color on the membrane, for example.

In some cases, PCR products can be quantified using a real-time thermocycler detection system. For example, Quantitative real-time PCR can use a fluorescent dye that forms a DNA-dye-complex (e.g., SYBR® Green), or a fluorophore-containing DNA probe, such as single-stranded oligonucleotides covalently bound to a fluorescent reporter or fluorophore (e.g. 6-carboxyfluorescein or tetrachlorofluorescin) and quencher (e.g., tetramethylrhodamine or dihydrocyclopyrroloindole tripeptide minor groove binder). The fluorescent signal allows detection of the amplified product in real time, thereby indicating the presence of a sequence of interest, and allowing quantification of the copy number of a sequence of interest in cellular DNA or expression level of a sequence of interest from cellular mRNA.

The identification of RFLPs is discussed, for example, in Alonso-Blanco et al. (Methods in Molecular Biology, vol. 82, “Arabidopsis Protocols”, pp. 137-146, J. M. Martinez-Zapater and J. Salinas, eds., c. 1998 by Humana Press, Totowa, N.J.); Burr (“Mapping Genes with Recombinant Inbreds”, pp. 249-254, in Freeling, M. and V. Walbot (Ed.), The Maize Handbook, c. 1994 by Springer-Verlag New York, Inc.: New York, N.Y., USA; Berlin Germany; Burr et al. Genetics (1998) 118: 519; and Gardiner, J. et al., (1993) Genetics 134: 917). For example, to produce a RFLP library enriched with single- or low-copy expressed sequences, total DNA can be digested with a methylation-sensitive enzyme (e.g., PstI). The digested DNA can be separated by size on a preparative gel. Polynucleotide fragments (500 to 2000 bp) can be excised, eluted and cloned into a plasmid vector (e.g., pUC18). Southern blots of plasmid digests can be probed with total sheared DNA to select clones that hybridize to single- and low-copy sequences. Additional restriction endonucleases can be tested to increase the number of polymorphisms detected.

The identification of AFLPs is discussed, for example, in EP 0 534 858 and U.S. Pat. No. 5,878,215. In general, total cellular DNA is digested with one or more restriction enzymes. Restriction halfsite-specific adapters are ligated to all restriction fragments and the fragments are selectively amplified with two PCR primers that have corresponding adaptor and restriction site specific sequences. The PCR products can be visualized after size-fractionation, as described above.

In some embodiments, the methods are directed to breeding a plant line. Such methods use genetic polymorphisms identified as described above in a marker assisted breeding program to facilitate the development of lines that have a desired alteration in the biomass trait. Once a suitable genetic polymorphism is identified as being associated with variation for the trait, one or more individual plants are identified that possess the polymorphic allele correlated with the desired variation. Those plants are then used in a breeding program to combine the polymorphic allele with a plurality of other alleles at other loci that are correlated with the desired variation. Techniques suitable for use in a plant breeding program are known in the art and include, without limitation, backcrossing, mass selection, pedigree breeding, bulk selection, crossing to another population and recurrent selection. These techniques can be used alone or in combination with one or more other techniques in a breeding program. Thus, each identified plants is selfed or crossed a different plant to produce seed which is then germinated to form progeny plants. At least one such progeny plant is then selfed or crossed with a different plant to form a subsequent progeny generation. The breeding program can repeat the steps of selfing or outcrossing for an additional 0 to 5 generations as appropriate in order to achieve the desired uniformity and stability in the resulting plant line, which retains the polymorphic allele. In most breeding programs, analysis for the particular polymorphic allele will be carried out in each generation, although analysis can be carried out in alternate generations if desired.

In some cases, selection for other useful traits is also carried out, e.g., selection for fungal resistance or bacterial resistance. Selection for such other traits can be carried out before, during or after identification of individual plants that possess the desired polymorphic allele.

### VII. Articles of Manufacture

Transgenic plants and modified plants provided herein have various uses in the agricultural and energy production industries. For example, transgenic plants and modified plants described herein can be used to make animal feed and food products. Such plants, however, are often particularly useful as a feedstock for energy production.

Transgenic plants and modified plants described herein often produce higher yields of grain and/or biomass per hectare, relative to control plants that lack the exogenous nucleic acid. In some embodiments, such plants provide equivalent or even increased yields of grain and/or biomass per hectare relative to control plants when grown under conditions of reduced inputs such as fertilizer and/or water. Thus, such transgenic and modified plants can be used to provide yield stability at a lower input cost and/or under environmentally stressful conditions such as drought. In some embodiments, plants described herein have a composition that permits more efficient processing into free sugars, and subsequently ethanol, for energy production. In some embodiments, such plants provide higher yields of ethanol, butanol, dimethyl ether, other biofuel molecules, and/or sugar-derived co-products per kilogram of plant material, relative to control plants. Such processing efficiencies are believed to be derived from the composition of the plant material, including, but not limited to, content of glucan, cellulose, hemicellulose, and lignin. By providing higher biomass yields at an equivalent or even decreased cost of production, the transgenic and modified plants described herein improve profitability for farmers and processors as well as decrease costs to consumers.

Seeds from plants described herein can be conditioned and bagged in packaging material by means known in the art to form an article of manufacture. Packaging material such as paper and cloth are well known in the art. A package of seed can have a label, e.g., a tag or label secured to the packaging material, a label printed on the packaging material, or a label inserted within the package, that describes the nature of the seeds therein.

The invention will be further described in the following examples, which do not limit the scope of the invention described in the claims.

### VIII. Examples

### Example 1

### Transgenic Switchgrass Exhibiting Decreased Ash Content

The T-DNA binary vectors containing an RNAi construct comprising SEQ ID NO:2914 or an RNAi construct comprising SEQ ID NO:2925 under the control of a PD3141 promoter (WO/2009/099899) were introduced into switchgrass by Agrobacterium-mediated transformation essentially as described in Richards et al., Plant Cell. Rep. 20:48-54 (2001) and Somleva et al., Crop Sci. 42:2080-2087 (2002). The nucleotide sequence set forth in SEQ ID NO:2914 corresponds to nucleic acid residues 22 to 325 of SEQ ID NO:877. The nucleotide sequence set forth in SEQ ID NO:2925 corresponds to the DNA complement of nucleic acid residues 23 to 412 of SEQ ID NO:2916. Plants from at least two independent events from each transformation were selected for further study; these events were referred to as switchgrass screening lines. The presence of each construct was confirmed by PCR.

The ash content of transgenic switchgrass lines was predicted using an NIR composition model for switchgrass. Aerial tissues (leaves and stems) were collected from T₀ plants 2 months after the transformants were transplanted into soil in the greenhouse. NIR spectral data were collected from dried, milled samples and entered into the NIR model. The predicted ash content of each transgenic line was compared to that of non-transgenic wild-type control plants. The percent ash content was also determined using a method that was substantially similar to the method described in ASTM E1755-01, “Standard Test Method for Ash in Biomass,” ASTM International, West Conshohocken, Pa., DOI: 10.1520/E1755-01R07. The results for wild-type and transgenic switchgrass lines are presented in Table 1.

TABLE 1

| | NIR Model | | ASTM E1755 | | |
|---|---|---|---|---|---|
| | | % Change | | | % Change |
| | | (relative to | | | (relative to |
| | | lowest | | | lowest |
| Plant Line | % Ash | WT value) | % Ash | STDev | WT value) |
| Pv00379-01 | 1.1 | −18.52 | 9.53 | 0.13 | −4.39 |
| Pv00379-02 | 0.83 | −38.52 | 9.21 | 0.01 | −7.56 |
| Pv00379-03 | 2.08 | 54.07 | 11.07 | 0.02 | 11.10 |
| Pv00379-04 | 0.91 | −32.59 | 9.43 | 0.09 | −5.40 |
| Pv00379-05 | 1.42 | 5.19 | 8.70 | 0.02 | −12.67 |
| Pv00379-06 | 0.99 | −26.67 | 8.25 | 0.01 | −17.25 |
| Pv00379-07 | 1.49 | 10.37 | 8.69 | 0.01 | −12.79 |
| Pv00379-08 | 0.57 | −57.78 | 8.87 | 0.05 | −11.05 |
| Pv00379-Ave | 1.17 | −13.06 | −9.22 | N/A | −7.51 |
| Pv00380-01 | 2.76 | 104.44 | 11.33 | 0.01 | 13.66 |
| Pv00380-02 | 2.87 | 112.59 | 10.75 | 0.01 | 7.81 |
| Pv00380-03 | 1.62 | 20.00 | 8.52 | 0.07 | −14.53 |
| Pv00380-04 | 2.38 | 76.30 | 8.92 | 0.10 | −10.52 |
| Pv00380-05 | 1.27 | −5.93 | 8.10 | 0.05 | −18.75 |
| Pv00380-06 | 2.2 | 62.96 | 7.63 | 0.02 | −23.40 |
| Pv00380-08 | 2.13 | 57.78 | 8.50 | 0.01 | −14.67 |
| Pv00380-09 | 0.89 | −34.07 | 8.84 | 0.06 | −11.31 |
| Pv00380-Ave | 2.02 | 49.26 | 9.07 | N/A | −8.96 |
| Pv-WT (A26)-21 | 1.35 | 0.00 | 10.73 | 0.13 | 7.65 |
| Pv-WT (A26)-22 | 2.34 | 73.33 | 10.48 | 0.01 | 5.12 |
| Pv-WT (A26)-23 | 1.97 | 45.93 | 11.70 | 0.02 | 17.41 |
| Pv-WT (A26)-24 | 3.4 | 151.85 | 12.51 | 0.12 | 25.56 |
| Pv-WT (A26)-25 | 1.74 | 28.89 | 10.74 | 0.05 | 7.79 |
| Pv-WT (A26)-26 | 2.34 | 73.33 | 9.97 | 0.02 | 0.00 |
| Pv-WT (A26)-27 | 1.44 | 6.67 | 10.25 | 0.04 | 2.79 |
| Pv-WT Ave | 2.08 | 54.29 | 10.91 | N/A | 9.47 |

These results indicate that the ash content of a transgenic plant as determined by ASTM E1755 can be decreased by about 4 percent to about 23 percent relative to the lowest value obtained for a wild-type control plant.

### Example 2

### Transgenic Arabidopsis Plants

The following is a list of nucleic acids that were isolated from Arabidopsis thaliana plants: CeresAnnot:831967 (SEQ ID NO:2879), CeresAnnot:837136 (SEQ ID NO:309), CeresClone:5055 (SEQ ID NO:402), CeresClone:14432 (SEQ ID NO:668), CeresAnnot:860791 (SEQ ID NO:2324), and CeresClone:21240 (SEQ ID NO:1796). The following nucleic acids were isolated from Panicum virgatum plants: CeresClone:1724846 (SEQ ID NO:834), CeresClone:1727475 (SEQ ID NO:335), CeresClone:1769660 (SEQ ID NO:1660), CeresClone:1775942 (SEQ ID NO:1137), CeresClone:1788006 (SEQ ID NO 1946), CeresClone:1801750 (SEQ ID NO:211), CeresClone:1806608 (SEQ ID NO:2544), CeresClone:1815770 (SEQ ID NO:2437), CeresClone:1817100 (SEQ ID NO:2090), CeresClone:1830065 (SEQ ID NO:2028), CeresClone:1821162 (SEQ ID NO:2323), CeresClone: 1797459 (SEQ ID NO:2619), CeresClone:1902646 (SEQ ID NO:501), CeresClone:1724055 (SEQ ID NO:1517), CeresClone:1724569 (SEQ ID NO:1623), CeresClone:1724782 (SEQ ID NO:1886), CeresClone:1725400 (SEQ ID NO:1317), CeresClone:1726076 (SEQ ID NO:458), CeresClone:1727628 (SEQ ID NO:845), CeresClone:1760764 (SEQ ID NO:1425), CeresClone:1763298 (SEQ ID NO:2524), CeresClone:1764605 (SEQ ID NO:1271), CeresClone:1773290 (SEQ ID NO:2653), CeresClone:1777248 (SEQ ID NO:2465), CeresClone:1781320 (SEQ ID NO:1106), CeresClone:1788124 (SEQ ID NO:1053), CeresClone:1788820 (SEQ ID NO:114), CeresClone:1789564 (SEQ ID NO:2707), CeresClone:1789593 (SEQ ID NO:1480), CeresClone:1790340 (SEQ ID NO:2747), CeresClone:1792586 (SEQ ID NO:1726), CeresClone:1794784 (SEQ ID NO:2145), CeresClone:1824159 (SEQ ID NO:1), CeresClone:1900097 (SEQ ID NO:1568), CeresClone:1955550 (SEQ ID NO:2189), CeresClone:1955766 (SEQ ID NO:748), CeresClone:1816881 (SEQ ID NO:1319), CeresClone:1888614 (SEQ ID NO:1116), CeresClone:1786326 (SEQ ID NO:2820), CeresClone:2010191 (SEQ ID NO:78), CeresClone:1809393 (SEQ ID NO:2598), CeresClone:1804242 (SEQ ID NO:628), and CeresClone:2014404 (SEQ ID NO:283). CeresClone:691319 (SEQ ID NO:692) was isolated from Glycine max plants. CeresClone:570057 (SEQ ID NO:716) was isolated from Triticum aestivum plants. CeresClone:1224770 (SEQ ID NO:2926) was isolated from Oryza sativa. CeresClone:240112 (SEQ ID NO:2620) was isolated from Zea mays plants.

Each isolated nucleic acid described above was cloned into a binary or cointegration Ti plasmid vector containing a selectable marker conferring resistance to Finale®. The nucleic acid was operably linked to a 35S promoter. Each vector was introduced into Arabidopsis ecotype Wassilewskija (WS) via Agrobacterium-mediated transformation using the floral dip method essentially as described in Bechtold, N. et al., C. R. Acad. Sci. Paris, 316:1194-1199 (1993). The presence of each construct was verified by PCR. At least two independent events from each transformation were selected for further study. T₀ plants are plants from transformed tissue culture. T₁ plants are first generation progeny of self-pollinated T₀ plants. T₂ plants are second generation progeny of self-pollinated T₁ plants.

### Example 3

### Transgenic Plants Exhibiting Increased Biomass

Biomass samples were collected and processed to determine the dry matter yield of transgenic plants. T₂ seeds from several transformation events of Arabidopsis were sown in a soil mixture composed of 60% Sunshine Mix and 40% medium-size vermiculite. Two lines were used as controls: (1) untransformed wild-type Arabidopsis and (2) wild-type transformed with construct Bin4-Ec6-Luc::NMCH-Luc, which contains a sequence conferring Finale® resistance, a Ec6 promoter to drive expression of a luciferase polypeptide, and a NMCH promoter to drive expression of a second luciferase polypeptide.

The sown seeds were vernalized for at least 3 days at 4° C. then transferred to either a greenhouse or a walk-in growth chamber for germination and growth. About one week post-germination, the experimental plants and the transgenic control were sprayed with Finale® to select for herbicide-resistant transgenic plants. Each pot was thinned-out by randomly removing seedlings to reduce the density of seedlings per pot to at least 10. Plants were allowed to grow until senescence.

Vegetative tissue (stems and undropped leaves) from ten senesced plants of each event were collected in a pre-labeled envelope and dried in a 42° C. oven overnight. Reproductive structures (siliques and seeeds) from senesced plants of each event were collected separately. After cooling, each sample of vegetative tissue was milled, processed for composition analysis, and weighed to obtain the dry matter yield for each event. The dry matter yield for plants of each transgenic event were compared to the corresponding value for control lines. The fold increase in dry matter yield relative to the control is presented in Table 2.

TABLE 2

| | | Dry Matter |
|---|---|---|
| | | Yield |
| Clone ID | Plant Line | (Fold Increase) |
| 1727475 | ME28945-01 | 1.40 |
| | ME28945-02 | 1.09 |
| | ME28945-03 | 1.36 |
| | ME28945-04 | 1.27 |
| | ME28945-05 | 1.19 |
| | ME28945-06 | 1.30 |
| 1788006 | ME29122-01 | 1.35 |
| | ME29122-02 | 1.24 |
| | ME29122-03 | 0.93 |
| | ME29122-04 | 0.97 |
| | ME29122-06 | 1.08 |
| 1806608 | ME29754-01 | 1.05 |
| | ME29754-02 | 0.87 |
| | ME29754-03 | 1.44 |
| | ME29754-04 | 1.48 |
| | ME29754-05 | 0.99 |
| | ME29754-06 | 1.34 |
| 1815770 | ME30349-01 | 2.15 |
| | ME30349-02 | 1.90 |
| | ME30349-03 | 2.74 |
| | ME30349-04 | 2.84 |
| | ME30349-05 | 2.72 |
| | ME30349-06 | 2.03 |
| 1775942 | ME30870-01 | 1.11 |
| | ME30870-02 | 0.98 |
| | ME30870-03 | 1.64 |
| | ME30870-04 | 1.12 |
| | ME30870-05 | 1.25 |
| | ME30870-06 | 1.57 |
| 1797459 | ME30884-01 | 1.08 |
| | ME30884-02 | 1.46 |
| | ME30884-04 | 1.29 |
| | ME30884-05 | 1.23 |
| 1821162 | ME31038-01 | 1.73 |
| | ME31038-02 | 1.45 |
| | ME31038-03 | 1.92 |
| | ME31038-04 | 2.10 |
| | ME31038-05 | 1.69 |
| 1769660 | ME27077-01 | 1.21 |
| | ME27077-02 | 1.33 |
| | ME27077-03 | 0.91 |
| | ME27077-04 | 1.21 |
| 1830065 | ME27681-01 | 1.16 |
| | ME27681-02 | 1.88 |
| | ME27681-03 | 1.23 |
| | ME27681-04 | 1.10 |
| | ME27681-05 | 1.20 |
| | ME27681-06 | 1.42 |
| 1790811 | ME27772-01 | 1.41 |
| | ME27772-02 | 1.01 |
| | ME27772-03 | 1.81 |
| | ME27772-05 | 1.60 |
| | ME27772-06 | 1.59 |
| 1724846 | ME29252-01 | 1.38 |
| | ME29252-02 | 1.03 |
| | ME29252-03 | 1.02 |
| | ME29252-05 | 1.33 |
| | ME29252-06 | 2.05 |
| 1817100 | ME29128-01 | 2.29 |
| | ME29128-02 | 2.55 |
| | ME29128-03 | 2.21 |
| | ME29128-04 | 2.08 |
| | ME29128-05 | 2.23 |
| | ME29128-06 | 2.89 |
| 1902646 | ME29277-01 | 5.48 |
| | ME29277-03 | 1.42 |
| | ME29277-04 | 1.99 |
| | ME29277-06 | 1.60 |
| 1801750 | ME26544-01 | 0.77 |
| | ME26544-02 | 0.66 |
| | ME26544-03 | 0.65 |
| | ME26544-04-99 | 1.21 |
| | ME26544-05 | 1.19 |
| | ME26544-06 | 1.19 |

These results indicate that the dry matter yield of a transgenic plant can be modulated relative to a control plant. As indicated above, the dry matter yield of a transgenic plant can be increased about 1.2 to about 5 fold, relative to a wild-type control plant.

### Example 4

### Transgenic Plants Exhibiting Increased Lignin

### NIR Model

Predicted lignin content of transgenic Arabidopsis thaliana lines was determined using a NIR composition model developed as described in WO2009/059176. Samples of biomass (typically at least 20 g dry weight) consisting of dried above-ground parts were collected from 10 to 12 T₂ plants at senescence. Samples were prepared for compositional analysis as described in “Preparation of Samples for Compositional Analysis,” September 2005, by the US Dept. of Energy National Renewable Energy Laboratory, which is substantially similar to ASTM method E1757-01. Briefly, samples were dried to a moisture content of 10% or less, either at room temperature or in a forced air oven set at or below 45° C. After drying, samples were milled in a standard laboratory knife mill to pass a 2 mm screen.

Near infrared spectral data were acquired from prepared samples using a Bruker Optics MPA™ FT-NIR spectrometer (Bruker Optics, Ettlingen, Germany), running Opus 5.5 software essentially according to the manufacturer's protocols. An integrating sphere with a rotating sample cup assembly was used to obtain spectra of samples. A standard lead-sulfide detector array was used to monitor NIR light from 12,800-5,800 cm-1. A Blackman-Harris 3-term apodization function was selected with a zero filling factor of 2. The Bruker instrument has a maximum resolution of 2 cm-1. To minimize the effect of water in the biomass spectra, each sample was air dried to less than 10% moisture prior to NIR analysis. Spectral information was collected from 12,500 cm-1 to 3600 cm-1 with a resolution of 8 cm⁻¹, and entered into the NIR model. The predicted lignin content of the transgenic line was compared to the predicted lignin content of the non-transgenic wild-type plants. The percent change in lignin (NIR) is presented in Table 3.

### Pyrolysis GC-MS Method

Samples of approximately 2 mg of milled dried biomass were analyzed on a Frontier lab PY-2020iS Pyrolyzer and Shimadzu QP2010 GCMS. Methylstearate was incorporated into the sample to serve as an internal standard. Samples were loaded and processed in a batch run with the following settings:

Py Settings:
Temperature: 550° C.
Interface: 300° C.

GC Settings:
Glass insert w/silica wool: SPLIT
Linear Velocity: 8.9 cm/sec
Split Ratio: 300
Injection Port Temperature: 350° C.
Oven Temperature: 70° C.
Detector Voltage: 0.5 kV
Interface Temperature: 250° C.
Acquisition Mode: Scan (for qualitative analysis)
Acquisition Time: 3.25-21.5 minutes, 35 m/z-400 m/z
Pressure: 128.8 kPa

MS Settings:
Ion Source Temperature: 200° C.
Interface Temperature: 250° C.
Solvent Cut time: 2.25 min
Micro scan width: 0

Chromatogram peaks were identified using the reference standards previously run during the protocol development. The area under each peak was manually integrated, quantified, and normalized relative to the internal standard. Levoglucosan and furfural served as cellulose markers. The average values for the lignin content of plants of each transgenic line were compared to the corresponding value for a wild-type control line. Events exhibiting increased lignin content are presented in Table 3.

TABLE 3

| | | % Change | % Change |
|---|---|---|---|
| | | in Lignin | in Lignin |
| Clone ID | Plant Line | (NIR) | (PyGC) |
| 1725400 | ME31528-01 | 17.81 | — |
| | ME31528-02 | 16.25 | — |
| | ME31528-03 | 21.91 | — |
| | ME31528-04 | 17.43 | — |
| 1724782 | ME26114-01 | 14.88 | 16.50 |
| | ME26114-02 | 15.16 | 30.21 |
| | ME26114-03 | 11.46 | 4.21 |
| | ME26114-04 | 13.10 | 7.08 |
| | ME26114-05 | 5.69 | −7.26 |
| | ME26114-06 | 10.60 | 10.41 |
| 1794784 | ME27658-01 | 10.44 | 17.02 |
| | ME27658-02 | 13.31 | −1.38 |
| | ME27658-03 | 12.78 | 3.13 |
| | ME27658-04 | 14.57 | 48.63 |
| | ME27658-05 | 12.33 | 42.45 |
| 1955766 | ME29626-01 | 4.10 | 67.14 |
| | ME29626-02 | −1.63 | 49.86 |
| | ME29626-03 | −4.57 | — |
| | ME29626-04 | 0.13 | 45.99 |
| | ME29626-05 | 3.88 | 36.71 |
| | ME29626-06 | −0.45 | 37.01 |
| 1804242 | ME26515-01 | 14.69 | 39.84 |
| | ME26515-03 | 16.19 | 39.38 |
| | ME26515-04 | 13.26 | 42.35 |
| | ME26515-05 | 15.84 | 53.77 |
| | ME26515-06 | 14.64 | 31.96 |
| 1816881 | ME28947-01 | 1.02 | 17.44 |
| | ME28947-02 | 10.60 | 15.11 |
| | ME28947-03 | 0.69 | 11.78 |
| | ME28947-04 | −2.15 | −7.44 |
| | ME28947-05 | −4.78 | −13.22 |
| | ME28947-06 | 9.32 | 12.22 |
| 570057 | ME24912-01 | 36.83 | 10.44 |
| | ME24912-02 | 31.66 | 14.22 |
| | ME24912-03 | 33.58 | 16.89 |
| | ME24912-05 | 32.35 | 13.56 |
| Annot | ME26166-01 | 26.37 | — |
| 831967 | ME26166-02 | 30.70 | 30.67 |
| | ME26166-03 | 24.76 | 1.44 |
| | ME26166-04 | 29.91 | 8.44 |
| | ME26166-05 | 28.86 | 34.00 |

— = Not Determined

These results indicate that the predicted lignin content of a transgenic plant can be increased from about 5 percent to about 37 percent, relative to a wild-type control plant.

### Example 5

### Transgenic Plants Exhibiting Decreased Lignin

Lignin content of transgenic Arabidopsis thaliana lines was determined as described in Example 4. Events exhibiting a decrease in lignin content are presented in Table 4.

TABLE 4

| | | % Change | % Change |
|---|---|---|---|
| | | in Lignin | in Lignin |
| Clone ID | Plant Line | (NIR) | (PyGC) |
| 2010191 | ME30683-01 | −10.12 | — |
| | ME30683-02 | −13.19 | — |
| | ME30683-03 | 3.71 | — |
| | ME30683-04 | −13.54 | — |
| | ME30683-05 | 4.66 | — |
| | ME30683-06 | 8.70 | — |
| 1786326 | ME30617-01 | 2.12 | — |
| | ME30617-02 | 4.37 | — |
| | ME30617-03 | 1.04 | — |
| | ME30617-04 | −4.50 | −8.18 |
| | ME30617-05 | −7.60 | −22.84 |
| | ME30617-06 | −7.12 | 7.16 |
| 21240 | ME25767-01 | −36.54 | — |
| | ME25767-02 | −43.68 | — |
| | ME25767-03 | −42.28 | — |
| | ME25767-04 | −52.38 | — |
| | ME25767-05 | −36.03 | — |
| | ME25767-06 | −24.86 | — |
| 21240 | ME26161-03 | −8.69 | — |
| | ME26161-04 | −14.98 | — |
| | ME26161-05 | −14.91 | — |
| | ME26161-06 | −10.04 | — |
| | ME26161-07 | 0.00 | — |
| 21240 | ME06492-02-99 | −16.30 | — |
| | ME06492-07-99 | −10.99 | — |
| 1777248 | ME30365-01 | −4.39 | — |
| | ME30365-02 | −10.27 | −11.35 |
| | ME30365-03 | −8.69 | −54.02 |
| Annot | ME11531-01 | −4.69 | −34.98 |
| 837136 | ME11531-03 | 1.92 | −15.73 |
| Annot | ME18822-01 | −16.56 | −30.85 |
| 860791 | ME18822-02 | −7.27 | −20.46 |
| 691319 | ME05057-05-A | −56.89 | −119.58 |
| | ME05057-06-A | −94.05 | — |
| | ME05057-07-A | −34.17 | −145.00 |
| 1224770 | ME25694-01 | 13.92 | −18.78 |
| | ME25694-02 | 6.49 | −14.56 |
| | ME25694-03 | 16.91 | −27.44 |
| | ME25694-04 | 13.59 | −26.22 |
| | ME25694-05 | −3.44 | −18.00 |

— = Not Determined

These results indicate that the predicted lignin content of a transgenic plant can be decreased by about 3 percent to about 90 percent, as compared to a wild-type control plant.

### Example 6

### Transgenic Plants Exhibiting an Increased S:G Ratio

To determine the amount of hydroxyphenyl (H) lignin monomers, the integrated normalized peaks corresponding to the following were added together: phenol, 2,6-dimethylphenol, 2-ethylphenol, 4-methylphenol, 3-ethylphenol, 4-vinylphenol, and 4-hydroxyacetophenone. To determine the amount of guaiacyl (G) lignin monomers, the integrated normalized peaks corresponding to the following were added together: guaiacol, 4-methylguaiacol, 4-ethylguaiacol, 4-vinylguaiacol, 5-propylguaiacol, vanillin, isovanillin, acetoguaiacone, guaiacylacetone, eugenol, isoeugenol, and coniferyaldehyde. To determine the amount of syringyl (S) lignin monomers, the integrated normalized peaks corresponding to the following were added together: syringol, 4-methylsyringol, 4-ethylsyringol, 4-vinylsyringol, 4-allysyringol, syringylacetone, acetosyringol, homosyringaldehyde, syringaldehyde, and sinapaldehyde. The sum of all the H, G, and S peaks correspond to the total lignin. The average values for the S:G ratio of plants of each transgenic line were compared to the corresponding values in wild-type control lines. Events exhibiting an increased S:G ratio are presented in Table 5.

TABLE 5

| | | % Change |
|---|---|---|
| | | in S:G Ratio |
| Clone ID | Plant Line | (PyGC) |
| 1790340 | ME29777-01 | 14.81 |
| | ME29777-02 | 14.81 |
| | ME29777-03 | 14.81 |
| | ME29777-04 | 18.52 |
| | ME29777-05 | 18.52 |
| | ME29777-06 | 18.52 |

These results indicate that the S:G ratio of a transgenic plant can be increased by about 15 percent to about 18 percent, relative to a wild-type control plant.

### Example 7

### Transgenic Plants Exhibiting Increased Glucan

Biomass samples from Arabidopsis thaliana plants were collected and processed for compositional analysis as described in Example 4. A NIR spectrum was acquired using a Bruker Optics MPA™ FT-NIR spectrometer (Bruker Optics, Ettlingen, Germany), running Opus 5.5 software essentially according to the manufacturer's protocols. The glucan content was predicted from the NIR spectra using a NIR composition model developed as described in WO2009/059176. The glucan content of the transgenic lines was compared to the glucan content of wild-type control lines. Events exhibiting increased glucan content are presented in Table

TABLE 6

| | | % Change in |
|---|---|---|
| | | Glucan |
| Clone ID | Plant Line | (NIR) |
| 1773290 | ME31545-01 | 12.83 |
| | ME31545-02 | 12.49 |
| | ME31545-03 | 11.07 |
| | ME31545-04 | 18.89 |
| | ME31545-05 | 6.42 |
| | ME31545-06 | 17.01 |
| 1888614 | ME31010-01 | 13.95 |
| | ME31010-02 | 16.55 |
| | ME31010-03 | 20.91 |
| | ME31010-04 | 11.76 |
| | ME31010-05 | 10.02 |
| | ME31010-06 | 9.47 |

These results indicate that the glucan content of a transgenic plant can be increased by about 6 percent to about 20 percent, relative to a wild-type control plant.

### Example 8

### Transgenic Plants Exhibiting Decreased Lignin and Increased Biomass

Lignin content of transgenic Arabidopsis thaliana lines was determined as described in Example 4. In addition, biomass samples were collected and dry matter yield determined as described in Example 3. The average values for lignin content and dry matter yield of plants of each transgenic line were compared to the corresponding values in wild-type control lines. Events with a predicted decrease in lignin content and an increase in the dry matter yield are presented in Table 7.

TABLE 7

| | | | Dry Matter |
|---|---|---|---|
| | | % Change | Yield |
| | | in Lignin | (Fold |
| Clone ID | Plant Line | (NIR) | Increase) |
| 1763298 | ME26843-01 | −24.46 | 0.92 |
| | ME26843-02 | −21.70 | 1.24 |
| | ME26843-03 | −8.98 | 1.69 |
| | ME26843-04 | −21.26 | 0.95 |
| | ME26843-05 | −20.76 | 1.22 |

These results indicate that the predicted lignin content and the dry matter yield of a transgenic plant can be modulated relative to a control plant. As indicated above, the predicted lignin content of a transgenic plant can be decreased by about 10 percent to about 25 percent, and concomitantly, the dry matter yield of the transgenic plant can be increased by about 0.9 fold to about 1.7 fold, relative to a wild-type control plant.

### Example 9

### Transgenic Plants Exhibiting Decreased Lignin and Increased S:G Ratio

Lignin content and S:G ratio of transgenic Arabidopsis thaliana lines were determined as described in Examples 4 and 6. The average values for lignin content and the S:G ratio of plants of each transgenic line were compared to the corresponding values for wild-type control plants. The percent change in the level of each component is presented in Table 8.

TABLE 8

| | | % Change | % Change | % Change |
|---|---|---|---|---|
| | | in Lignin | in Lignin | in S:G Ratio |
| Clone ID | Plant Line | (NIR) | (PyGC) | (PyGC) |
| 1809393 | ME27043-01 | −30.12 | −30.28 | 9.83 |
| | ME27043-02 | −24.52 | −40.01 | 19.76 |
| | ME27043-03 | −32.86 | −40.96 | 7.08 |
| | ME27043-04 | −19.95 | — | — |
| | ME27043-05 | −22.79 | — | — |

— = Not Determined

These results show that the predicted lignin content and S:G ratio of a transgenic plant can be modulated relative to a control plant. As indicated above, the predicted lignin content of a transgenic plant can be decreased from about 25 percent to about 30 percent, and concomitantly, the S:G ratio of the transgenic plant can be increased by about 7 percent to about 20 percent, relative to a control plant.

### Example 10

### Transgenic Plants Exhibiting Increased Glucan and Increased S:G Ratio

Glucan content and the S:G ratio of transgenic Arabidopsis thaliana lines were determined as described in Examples 6 and 7. The average values for glucan content and the S:G ratio of plants of each transgenic line were compared to the corresponding values for wild-type control plants. Events exhibiting increased glucan content and increased S:G ratio are presented in Table 9.

TABLE 9

| | | % Change in | % Change |
|---|---|---|---|
| | | Glucan | in S:G Ratio |
| Clone ID | Plant Line | (NIR) | (PyGC) |
| 1789564 | ME29970-01 | 16.04 | 14.04 |
| | ME29970-02 | 14.11 | 9.09 |
| | ME29970-03 | 12.82 | 7.49 |
| | ME29970-04 | 2.45 | 13.14 |
| | ME29970-06 | 10.59 | 4.88 |

These results indicate that the glucan content and S:G ratio of a transgenic plant can be modulated relative to a control plant. As indicated above, the glucan content of a transgenic plant can be increased by about 2 percent to about 16 percent, and concomitantly, the S:G ratio of the transgenic plant can be increased from about 5 percent to about 14 percent, relative to a control plant.

### Example 11

### Transgenic Plants Exhibiting Increased Glucan and Increased Lignin

Lignin and glucan content of transgenic Arabidopsis thaliana lines were determined as described in Examples 4 and 7. The average values for glucan content and lignin content of plants of each transgenic line were compared to the corresponding values for wild-type control plants. Events exhibiting increased glucan and increased lignin content are presented in Table 10.

TABLE 10

| | | % Change in | % Change | % Change |
|---|---|---|---|---|
| | | Glucan | in Lignin | in Lignin |
| Clone ID | Plant Line | (NIR) | (NIR) | (PyGC) |
| 1764605 | ME31173-01 | 15.58 | −2.93 | 20.50 |
| | ME31173-03 | 15.91 | 3.43 | 40.09 |
| | ME31173-04 | 15.62 | 5.61 | 38.71 |
| | ME31173-05 | 16.66 | 7.26 | 20.04 |
| | ME31173-06 | 16.33 | 0.53 | 15.86 |

These results indicate that the glucan content and the predicted lignin content of a transgenic plant can be modulated relative to control plant. As indicated above, the glucan content of a transgenic plant can be increased by about 15 percent, and concomitantly, the predicted lignin content of the transgenic plant can be increased by about 1 percent to about 7 percent, relative to a wild-type control plant.

### Example 12

### Transgenic Plants Exhibiting Increased Lignin and Increased S:G Ratio

Lignin content and the S:G ratio of transgenic Arabidopsis thaliana lines were determined as described in Examples 4 and 6. The average values for lignin content and S:G ratio of plants of each transgenic line were compared to the corresponding values for wild-type control plants. Events exhibiting increased lignin and decreased S:G ratio are presented in Table 11.

TABLE 11

| | | % Change | % Change | % Change |
|---|---|---|---|---|
| | | in Lignin | in Lignin | in S:G Ratio |
| Clone ID | Plant Line | (NIR) | (PyGC) | (PyGC) |
| 1727628 | ME27144-01 | 16.41 | 85.59 | −3.27 |
| | ME27144-02 | 16.19 | 77.90 | −11.01 |
| | ME27144-03 | 11.64 | 71.13 | −6.76 |
| | ME27144-04 | 14.93 | 113.24 | −12.39 |
| | ME27144-05 | 17.76 | 89.80 | −14.38 |
| | ME27144-06 | 11.78 | 68.27 | −9.91 |

These results indicate that the predicted lignin content and the S:G ratio of a transgenic plant can be modulated relative to a control plant. As indicated above, the predicted lignin content of a transgenic plant can be increased by about 12 percent to about 18 percent, and concomitantly, the S:G ratio of the transgenic plant can be decreased by about 3 percent to about 14 percent, relative to a control plant.

### Example 13

### Transgenic Plants Exhibiting Increased Lignin and Increased Biomass

Lignin content and the dry matter yield of transgenic Arabidopsis thaliana lines were determined as described in Examples 4 and 3. The average values for lignin content and the dry matter yield of plants of each transgenic line were compared to the corresponding values for wild-type control plants. Events exhibiting increased lignin content and increased dry matter yield are presented in Table 12.

TABLE 12

| | | | | Dry Matter |
|---|---|---|---|---|
| | | % Change | % Change | Yield |
| | | in Lignin | in Lignin | (Fold |
| Clone ID | Plant Line | (NIR) | (PyGC) | Increase) |
| 1726076 | ME29794-02 | 3.92 | 36.46 | 2.06 |
| | ME29794-03 | 6.27 | 35.30 | 1.76 |
| | ME29794-04 | 1.77 | 30.32 | 1.33 |
| | ME29794-05 | 1.95 | 12.41 | 1.67 |
| | ME29794-06 | 7.52 | 6.78 | 2.01 |
| 1788820 | ME30345-01 | 11.53 | −3.64 | 1.67 |
| | ME30345-02 | 4.79 | −2.40 | 1.58 |
| | ME30345-03 | 9.58 | 2.48 | 1.22 |
| | ME30345-04 | 7.21 | 11.75 | 1.99 |
| | ME30345-05 | 8.94 | 10.59 | 1.78 |
| | ME30345-06 | 5.63 | 2.81 | 1.61 |

These results indicate that the predicted lignin content and the dry matter yield of a transgenic plant can be modulated. As indicated above, the predicted lignin content of a transgenic plant can be increased by about 2 percent to about 12 percent, and concomitantly, the dry matter yield of the transgenic plant can be increased by about 2 fold, relative to a wild-type control plant.

### Example 14

### Transgenic Plants Exhibiting Decreased Lignin and Increased S:G Ratio

Lignin content and the S:G ratio of transgenic Arabidopsis thaliana lines were determined as described in Examples 4 and 6. The average values for lignin content and S:G ratio of plants of each transgenic line were compared to the corresponding values for wild-type control plants. The percent change in each component is presented in Table 13.

TABLE 13

| | | % Change | % Change | % Change |
|---|---|---|---|---|
| | | in Lignin | in Lignin | in S:G Ratio |
| Clone ID | Plant Line | (NIR) | (PyGC) | (PyGC) |
| 2014404 | ME30110-01 | −3.84 | −37.73 | 64.29 |
| | ME30110-02 | 4.91 | −34.86 | 14.29 |
| | ME30110-03 | −5.06 | −60.50 | 64.29 |
| | ME30110-04 | −6.80 | −49.89 | 60.71 |
| | ME30110-05 | −0.77 | −47.46 | 28.57 |
| | ME30110-06 | −7.07 | −59.03 | 35.71 |

These results indicate that the predicted lignin content and the S:G ratio of a transgenic plant can be modulated relative to a control plant. As indicated above, the predicted lignin content of a transgenic plant can be decreased by about 1 percent to about 7 percent, and concomitantly the S:G ratio of the transgenic plant can be increased by about 14 percent to about 60 percent, relative to a wild-type control plant.

### Example 15

### Transgenic Plants Exhibiting Increased Lignin Content and Decreased S:G Ratio

Lignin content and the S:G ratio of transgenic Arabidopsis thaliana lines were determined as described in Examples 4 and 6. The average values for lignin content and S:G ratio of plants of each transgenic line were compared to the corresponding values for wild-type control plants. The percent change in the level of each component is presented in Table 14.

TABLE 14

| | | % Change | % Change | % Change |
|---|---|---|---|---|
| | | in Lignin | in Lignin | in S:G Ratio |
| Clone ID | Plant Line | (NIR) | (PyGC) | (PyGC) |
| 1955550 | ME30330-01 | 2.92 | 1.49 | −3.23 |
| | ME30330-02 | −4.81 | 8.68 | 0.00 |
| | ME30330-03 | 13.88 | 17.37 | −9.68 |
| | ME30330-04 | 9.43 | 25.72 | −6.45 |
| | ME30330-05 | 5.02 | 13.98 | −12.90 |
| | ME30330-06 | 8.54 | 9.10 | −12.90 |

These results indicate that the predicted lignin content and S:G ratio of a transgenic plant can be modulated relative to a control plant. As indicated above, the predicted lignin content of a transgenic plant can be increased by about 3 percent to about 14 percent, and concomitantly, the S:G ratio of the transgenic plant can be decreased by about 3 percent to about 13 percent, relative to a wild-type control plant.

### Example 16

### Transgenic Plants Exhibiting Decreased Lignin and Increased Biomass

Lignin content and dry matter yield of transgenic Arabidopsis thaliana lines were determined as described in Examples 4 and 3. The average values for lignin content and dry matter yield of plants of each transgenic line were compared to the corresponding values for wild-type control plants. The events exhibiting decreased lignin content and increased dry matter yield are presented in Table 15.

TABLE 15

| | | | | Dry Matter |
|---|---|---|---|---|
| | | % Change | % Change | Yield |
| | | in Lignin | in Lignin | (Fold |
| Clone ID | Plant Line | (NIR) | (PyGC) | Increase) |
| 1789593 | ME28415-01 | 9.89 | 5.29 | — |
| | ME28415-02 | 6.04 | −20.18 | 1.26 |
| | ME28415-03 | 2.77 | −2.81 | — |
| | ME28415-04 | 1.91 | −53.10 | 1.54 |
| | ME28415-05 | −3.22 | −2.81 | 1.31 |
| | ME28415-06 | 2.46 | 3.97 | — |

— = Not Determined

These results indicate that the predicted lignin content and the dry matter yield of a transgenic plant can be modulated relative to a control plant. As indicated above, the lignin content of a transgenic plant can be increased by about 3 percent, and concomitantly, the dry matter yield of the transgenic plant can be increased by about 1.5 fold.

### Example 17

### Transgenic Plants Exhibiting Increased Glucan Content and Decreased Lignin Content

Glucan and lignin content of transgenic Arabidopsis thaliana lines were determined as described in Examples 4 and 7. The average values for glucan and lignin content of plants of each transgenic line were compared to the corresponding values for wild-type control plants. The events exhibiting increased glucan content and decreased lignin content are presented in Table 16.

TABLE 16

| | | % Change in | % Change | % Change |
|---|---|---|---|---|
| | | Glucan | in Lignin | in Lignin |
| Clone ID | Plant Line | (NIR) | (NIR) | (PyGC) |
| 14432 | ME07523-04 | 7.89 | −16.85 | −21.68 |
| 14432 | ME04241-04-B | 53.94 | −3.43 | −35.32 |
| | ME04241-05-D | 48.44 | −11.65 | −40.43 |

These results indicate that the glucan content and the predicted lignin content of a transgenic plant can be modulated relative to a control plant. As indicated above, the glucan content can be increased from about 48 percent to about 54 percent, and concomitantly, the lignin content of the transgenic plant can be decreased by about 3 percent to about 17 percent, relative to a wild-type control plant.

### Example 18

### Transgenic Plants Exhibiting Decreased Glucan and Increased Lignin

Glucan and lignin content of transgenic Arabidopsis thaliana lines were determined as described in Examples 4 and 7. The average values for the glucan and lignin content of plants of each transgenic line were compared to the corresponding values for wild-type control plants. The events exhibiting decreased glucan content and increased lignin content are presented in Table 17.

TABLE 17

| | | % Change in | % Change | % Change |
|---|---|---|---|---|
| | | Glucan | in Lignin | in Lignin |
| Clone ID | Plant Line | (NIR) | (NIR) | (PyGC) |
| 1824159 | ME27368-01 | −11.95 | 5.34 | — |
| | ME27368-02 | −9.86 | 8.05 | — |
| | ME27368-04 | −13.08 | 3.57 | 46.67 |
| | ME27368-05 | −15.04 | 3.57 | 19.33 |
| | ME27368-06 | −12.45 | −2.77 | −5.33 |

— = Not Determined

These results indicate that the glucan content and the predicted lignin content of a transgenic plant can be modulated relative to a control plant. As indicated above, the glucan content can be decreased by about 10 percent to about 15 percent, and concomitantly, the lignin content of the transgenic plant can be increased by about 4 percent to about 8 percent, relative to a wild-type control plant.

### Example 19

### Transgenic Plants Exhibiting Increased Lignin and Increased Biomass

Lignin content and the dry matter yield of transgenic Arabidopsis thaliana lines were determined as described in Examples 4 and 3. The average values for lignin content and the dry matter yield of plants of each transgenic line were compared to the corresponding values for wild-type control plants. The events exhibiting increased dry matter yield and lignin content are presented in Table 18.

TABLE 18

| | | | | Dry Matter |
|---|---|---|---|---|
| | | % Change | % Change | Yield |
| | | in Lignin | in Lignin | (Fold |
| Clone ID | Plant Line | (NIR) | (PyGC) | Increase) |
| 1724055 | ME29158-01 | 33.39 | 54.33 | 0.99 |
| | ME29158-02 | 24.94 | 47.89 | 0.91 |
| | ME29158-03 | 34.04 | 35.11 | 0.82 |
| | ME29158-04 | 34.70 | 100.33 | 1.24 |
| | ME29158-05 | 43.71 | 52.44 | 1.61 |
| | ME29158-06 | 55.25 | 94.78 | 0.92 |

These results indicate that the predicted lignin content and the dry matter yield of a transgenic plant can be modulated relative to a control plant. As indicated above, the predicted lignin content of a transgenic plant can be increased by about 25 percent to about 55 percent, and concomitantly, the dry matter yield of the transgenic plant can be increased by about 1.6 fold.

### Example 20

### Transgenic Plants Exhibiting Decreased Lignin, Increased S:G Ratio, and Increased Biomass

Lignin content, the S:G ratio, and the dry matter yield of transgenic Arabidopsis thaliana lines were determined as described in Examples 3, 4 and 6. The average values for lignin content, S:G ratio, and the dry matter yield of plants of each transgenic line were compared to the corresponding value for wild-type control plants. The events exhibiting decreased lignin content, increased S:G ratio, and increased dry matter yield are presented in Table 19.

TABLE 19

| | | | | | Dry |
|---|---|---|---|---|---|
| | | | | % Chang | Matter |
| | | % Change | % Change | e in | Yield |
| | | in Lignin | in Lignin | S:G Ratio | (Fold |
| Clone ID | Plant Line | (NIR) | (PyGC) | (PyGC) | Increase) |
| 1900097 | ME30392-01 | −18.31 | −17.43 | −8.67 | 0.93 |
| | ME30392-02 | −19.62 | 0.10 | −2.67 | 1.38 |
| | ME30392-03 | −25.49 | −8.54 | 11.79 | 1.44 |
| | ME30392-04 | −17.53 | −10.58 | 11.30 | 1.42 |
| | ME30392-05 | −23.30 | −12.92 | 15.10 | 1.25 |
| | ME30392-06 | −18.63 | −11.71 | 8.24 | 0.94 |

These results indicate the predicted lignin content, the S:G ratio, and the dry matter yield of a transgenic plant can be modulated relative to a control plant. As indicated above, the lignin content of a transgenic plant can be decreased by about 18 percent to about 25 percent, the S:G ratio of the transgenic plant can be increased by about 8 percent to about 15 percent, and concomitantly, the dry matter yield of the transgenic plant can be increased by about 1.4 fold, relative to a wild-type control plant.

### Example 21

### Transgenic Plants Exhibiting Increased Lignin, Decreased S:G Ratio and Increased Biomass

Lignin content, the S:G ratio, and the dry matter yield of transgenic Arabidopsis thaliana lines were determined as described in Examples 3, 5, and 6. The average values for lignin content, the S:G ratio, and the dry matter yield of plants of each transgenic line were compared to the corresponding values for wild-type control plants. The events exhibiting increased lignin content, decreased S:G ratio, and increased dry matter yield are presented in Table 20.

TABLE 20

| | | | | | Dry |
|---|---|---|---|---|---|
| | | | | % Change | Matter |
| | | % Change | % Change | in | Yield |
| | | in Lignin | in Lignin | S:G Ratio | (Fold |
| Clone ID | Plant Line | (NIR) | (PyGC) | (PyGC) | Increase) |
| 1781320 | ME29764-01 | 15.59 | 79.35 | −22.93 | 1.23 |
| | ME29764-02 | 13.36 | 66.87 | −19.08 | 1.29 |
| | ME29764-03 | 15.15 | 81.11 | −0.32 | — |
| | ME29764-04 | 16.69 | 83.52 | −13.69 | — |
| | ME29764-05 | 17.91 | 85.47 | −18.46 | — |
| | ME29764-06 | 15.40 | 68.53 | −12.40 | — |

— = Not Determined

These results indicate that the predicted lignin content, S:G ratio, and dry matter yield of a transgenic plant can be modulated relative to a control plant. As indicated above, the lignin content of a transgenic plant can be increased by about 13 percent to about 18 percent, the S:G ratio of the transgenic plant can be decreased by about 1 percent to about 23 percent, and concomitantly, the dry matter yield of the transgenic plant can be increased by about 1.3 fold, relative to a wild-type control plant.

### Example 22

### Transgenic Plants Exhibiting Increased Lignin, Increased S:G Ratio, and Increased Biomass

Lignin content, the S:G ratio, and the dry matter yield of transgenic Arabidopsis thaliana lines were determined as described in Examples 3, 4, and 6. The average values for lignin content, S:G ratio, and the dry matter yield of plants of each transgenic line were compared to the corresponding values for wild-type control plants. The events exhibiting increased lignin content, increased S:G ratio, and increased dry matter yield are presented in Table 21.

TABLE 21

| | | | | | Dry |
|---|---|---|---|---|---|
| | | | | % Change | Matter |
| | | % Change | % Change | in | Yield |
| | | in Lignin | in Lignin | S:G Ratio | (Fold |
| Clone ID | Plant Line | (NIR) | (PyGC) | (PyGC) | Increase) |
| 1724569 | ME26627-01 | 11.37 | 24.77 | 20.00 | 1.39 |
| | ME26627-02 | 12.11 | 16.32 | 8.00 | 1.18 |
| | ME26627-03 | 14.13 | 48.40 | 12.00 | — |
| | ME26627-04 | 11.16 | 14.95 | 4.00 | — |
| | ME26627-05 | 12.61 | 16.10 | 12.00 | — |

— = Not Determined

These results indicate that the predicted lignin content, the S:G ratio, and the dry matter yield of a transgenic plant can be modulated relative to a control plant. As indicated above, the predicted lignin content of a transgenic plant can be increased by about 11 percent to about 14 percent, the S:G ratio of the transgenic plant can be increased by about 4 percent to about 20 percent, and concomitantly, the dry matter yield of the transgenic plant can be increased by about 1.4 fold, relative to a wild-type control plant.

### Example 23

### Transgenic Plants Exhibiting Increased Glucan, Increased Lignin, and Increased Biomass

Glucan content, lignin content, and the dry matter yield of transgenic Arabidopsis thaliana lines were determined as described in Examples 3, 4, and 7. The average values for glucan content, lignin content, and the dry matter yield of plants of each transgenic line were compared to the corresponding values for wild-type control plants. The events exhibiting increased glucan content, increased lignin content, and increased dry matter yield are presented in Table 22.

TABLE 22

| | | | | | Dry |
|---|---|---|---|---|---|
| | | % Change | | | Matter |
| | | in | % Change | % Change | Yield |
| | | Glucan | in Lignin | in Lignin | (Fold |
| Clone ID | Plant Line | (NIR) | (NIR) | (PyGC) | Increase) |
| 1788124 | ME31183-01 | 16.11 | 8.57 | 31.02 | 0.43 |
| | ME31183-02 | 20.08 | 6.95 | 7.82 | 0.29 |
| | ME31183-03 | 4.85 | −8.31 | 21.37 | 1.46 |
| | ME31183-04 | 3.92 | −17.39 | 15.52 | 1.50 |
| | ME31183-05 | 7.79 | −18.01 | 14.19 | 1.73 |
| | ME31183-06 | 2.95 | −16.43 | 20.32 | 1.92 |

These results indicate that the glucan content, the predicted lignin content, and the dry matter yield of a transgenic plant can be modulated relative to a control plant. As indicated above, the glucan content of a transgenic plant can be increased from about 4 percent to about 20 percent, the predicted lignin content of the transgenic plant can be increased from about 7 percent to about 9 percent, and concomitantly, the dry matter yield of the transgenic plant can be increased about 2 fold, relative to a wild-type control plant.

### Example 24

### Transgenic Plants Exhibiting Increased Glucan, Increased Lignin, and Increased S:G Ratio

Glucan content, lignin content, and the S:G ratio of transgenic Arabidopsis thaliana lines were determined as described in Examples 4, 6, and 7. The average values for glucan content, lignin content, and S:G ratio of plants of each transgenic line were compared to the corresponding values for wild-type control plants. The events exhibiting increased glucan content, decreased lignin content, and increased S:G ratio are presented in Table 23.

TABLE 23

| | | | % | % | % |
|---|---|---|---|---|---|
| | | | Change | Change | Change |
| | | % Change | in | in | in |
| | | in Glucan | Lignin | Lignin | S:G Ratio |
| Clone ID | Plant Line | (NIR) | (NIR) | (PyGC) | (PyGC) |
| 5055 | ME03563-01-99 | 1.75 | 13.82 | −32.19 | 22.22 |
| | ME03563-03-99 | 39.54 | 33.44 | −60.78 | 29.63 |
| | ME03563-05-99 | 12.15 | 13.93 | −56.17 | 25.93 |
| | ME03563-06-99 | 2.14 | 22.07 | −57.69 | 18.52 |
| 1760764 | ME31185-02 | 8.52 | −17.26 | −16.01 | 7.76 |
| | ME31185-03 | 5.67 | −25.50 | −36.09 | 21.01 |
| | ME31185-04 | 19.31 | −18.29 | −7.21 | 12.45 |
| | ME31185-05 | 23.12 | −12.38 | −6.03 | 18.90 |
| | ME31185-06 | 23.78 | −6.05 | 9.64 | 5.51 |

These results indicate that the glucan content, the predicted lignin content, and the S:G ratio of a transgenic plant can be modulated relative to a control plant. As indicated above, the glucan content of a transgenic plant can be increased by about 2 percent to about 40 percent, the predicted lignin content of the transgenic plant can be decreased by about 6 percent to about 26 percent, and concomitantly, the S:G ratio of the transgenic plant can be increased by about 6 percent to about 30 percent, relative to a wild-type control plant.

### Example 25

### Transgenic Plants Exhibiting Decreased Glucan, Decreased Lignin, Increased S:G ratio and Increased Biomass

Glucan and lignin content, the S:G ratio, and the dry matter yield of transgenic Arabidopsis thaliana lines were determined as described in Examples 3, 4, 6, and 7. The average values for glucan and lignin content, S:G ratio, and the dry matter yield of plants of each transgenic line were compared to the corresponding values for wild-type control plants. The events exhibiting decreased glucan content, decreased lignin content, increased S:G ratio, and increased dry matter yield are presented in Table 24.

TABLE 24

| | | | | | | Dry Matter |
|---|---|---|---|---|---|---|
| | | % Change in | % Change | % Change | % Change | Yield |
| | | Glucan | in Lignin | in Lignin | in S:G Ratio | (Fold |
| Clone ID | Plant Line | (NIR) | (NIR) | (PyGC) | (PyGC) | Increase) |
| 1792586 | ME28309-01 | −16.22 | −14.70 | −39.14 | 14.24 | 1.41 |
| | ME28309-02 | −13.37 | −4.39 | −35.22 | 25.87 | 1.55 |
| | ME28309-03 | −13.54 | −5.68 | −24.21 | 10.85 | 1.69 |
| | ME28309-04 | −16.51 | −11.53 | −48.23 | 38.73 | 2.23 |
| | ME28309-05 | −9.45 | −14.04 | −26.42 | 5.58 | 2.18 |
| | ME28309-06 | −10.86 | −8.70 | −25.77 | 26.05 | 2.08 |
| 240112 | ME29422-01 | −12.30 | −1.90 | −20.47 | −4.02 | 0.99 |
| | ME29422-02 | −17.95 | −6.39 | −42.58 | 17.35 | 0.85 |
| | ME29422-04 | −13.06 | 0.56 | −38.06 | 12.74 | 1.23 |
| | ME29422-05 | −19.23 | −9.55 | −43.30 | 26.53 | 1.14 |
| | ME29422-06 | 0.41 | 3.85 | −9.88 | 16.65 | 1.61 |

These results indicate that the glucan content, the predicted lignin content, the S:G ratio, and the dry matter yield of a transgenic plant can be modulated relative to a control plant. As indicated above, the glucan level of a transgenic plant can be decreased by about 9 percent to about 19 percent, the predicted lignin content of the transgenic plant can be decreased by about 2 percent to about 15 percent, the S:G ratio of the transgenic plant can be increased by about 6 percent to about 39 percent, and concomitantly, the dry matter yield of the transgenic plant can be increased by about 2 fold.

### Example 26

### Determination of Functional Homologs by Reciprocal BLAST

A candidate sequence was considered a functional homolog of a reference sequence if the candidate and reference sequences encoded proteins having a similar function and/or activity. A process known as Reciprocal BLAST (Rivera et al., Proc. Natl. Acad. Sci. USA, 95:6239-6244 (1998)) was used to identify potential functional homolog sequences from databases consisting of all available public and proprietary peptide sequences, including NR from NCBI and peptide translations from Ceres clones.

Before starting a Reciprocal BLAST process, a specific reference polypeptide was searched against all peptides from its source species using BLAST in order to identify polypeptides having BLAST sequence identity of 80% or greater to the reference polypeptide and an alignment length of 85% or greater along the shorter sequence in the alignment. The reference polypeptide and any of the aforementioned identified polypeptides were designated as a cluster.

The BLASTP version 2.0 program from Washington University at Saint Louis, Mo., USA was used to determine BLAST sequence identity and E-value. The BLASTP version 2.0 program includes the following parameters: 1) an E-value cutoff of 1.0e-5; 2) a word size of 5; and 3) the -postsw option. The BLAST sequence identity was calculated based on the alignment of the first BLAST HSP (High-scoring Segment Pairs) of the identified potential functional homolog sequence with a specific reference polypeptide. The number of identically matched residues in the BLAST HSP alignment was divided by the HSP length, and then multiplied by 100 to get the BLAST sequence identity. The HSP length typically included gaps in the alignment, but in some cases gaps were excluded.

The main Reciprocal BLAST process consists of two rounds of BLAST searches; forward search and reverse search. In the forward search step, a reference polypeptide sequence, “polypeptide A,” from source species SA was BLASTed against all protein sequences from a species of interest. Top hits were determined using an E-value cutoff of 10⁻⁵ and a sequence identity cutoff of 35%. Among the top hits, the sequence having the lowest E-value was designated as the best hit, and considered a potential functional homolog or ortholog. Any other top hit that had a sequence identity of 80% or greater to the best hit or to the original reference polypeptide was considered a potential functional homolog or ortholog as well. This process was repeated for all species of interest.

In the reverse search round, the top hits identified in the forward search from all species were BLASTed against all protein sequences from the source species SA. A top hit from the forward search that returned a polypeptide from the aforementioned cluster as its best hit was also considered as a potential functional homolog.

Functional homologs were identified by manual inspection of potential functional homolog sequences. Representative functional homologs for SEQ ID NO:2880, 310, 403, 669, 1797, 717, 693, 1518, 1624, 1887, 834, 1318, 459, 335, 846, 1426, 2525, 1272, 878, 1660, 2654, 1137, 2466, 1107, 1946, 1054, 115, 2708, 1481, 2748, 947, 1727, 2146, 2619, 211, 629, 2544, 2599, 2437, 2090, 2323, 2, 2028, 1569, 501, 2190, 749, 79, 284, and 2915 are shown in FIGS. 1-50, respectively. Additional exemplary homologs are correlated to certain Figures in the Sequence Listing.

### Example 27

### Determination of Functional Homologs by Hidden Markov Models

Hidden Markov Models (HMMs) were generated by the program HMMER 2.3.2. To generate each HMM, the default HMMER 2.3.2 program parameters, configured for global alignments, were used.

An HMM was generated using the sequences shown in FIG. 1 as input. These sequences were fitted to the model and a representative HMM bit score for each sequence is shown in the Sequence Listing. Additional sequences were fitted to the model, and representative HMM bit scores for any such additional sequences are shown in the Sequence Listing. The results indicate that these additional sequences are functional homologs of SEQ ID NO:2880.

The procedure above was repeated and an HMM was generated for each group of sequences shown in FIGS. 2-50, using the sequences shown in each Figure as input for that HMM. A representative bit score for each sequence is shown in the Sequence Listing. Additional sequences were fitted to certain HMMs, and representative HMM bit scores for such additional sequences are shown in the Sequence Listing. The results indicate that these additional sequences are functional homologs of the sequences used to generate that HMM.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims

1. A method of producing a plant, said method comprising growing a plant cell comprising an exogenous nucleic acid, said exogenous nucleic acid comprising a regulatory region operably linked to a nucleotide sequence encoding a polypeptide, wherein the HMM bit score of the amino acid sequence of said polypeptide is greater than about 93, said HMM based on the amino acid sequences depicted in one of FIGS. 1-50, and wherein said plant has a difference in the level of biomass as compared to the corresponding level of a control plant that does not comprise said nucleic acid.

2. A method of producing a plant, said method comprising growing a plant cell comprising an exogenous nucleic acid, said exogenous nucleic acid comprising a regulatory region operably linked to a nucleotide sequence encoding a polypeptide having 80 percent or greater sequence identity to an amino acid sequence selected from the group consisting of SEQ ID NO:2, 4, 5, 6, 8, 10, 12, 13, 15, 17, 18, 20, 22, 24, 25, 26, 27, 29, 30, 32, 34, 35, 37, 38, 39, 41, 43, 45, 47, 49, 51, 53, 55, 56, 57, 59, 60, 62, 63, 64, 65, 67, 69, 70, 71, 73, 74, 75, 77, 79, 80, 81, 82, 83, 84, 85, 86, 88, 90, 91, 92, 93, 94, 95, 96, 97, 98, 99, 100, 101, 102, 103, 104, 105, 106, 107, 108, 109, 110, 111, 112, 113, 115, 117, 119, 120, 121, 122, 123, 124, 126, 128, 129, 131, 133, 135, 137, 139, 140, 141, 142, 144, 146, 147, 148, 149, 150, 152, 154, 155, 157, 158, 160, 162, 163, 165, 167, 169, 171, 173, 175, 176, 178, 180, 182, 183, 185, 187, 189, 191, 193, 194, 195, 196, 197, 198, 199, 200, 201, 203, 205, 207, 208, 209, 211, 213, 214, 215, 217, 218, 219, 220, 222, 223, 224, 226, 228, 229, 231, 232, 233, 234, 235, 236, 237, 238, 239, 240, 242, 244, 245, 246, 247, 248, 249, 250, 251, 252, 254, 255, 256, 257, 258, 259, 260, 261, 262, 263, 264, 265, 266, 267, 268, 269, 270, 271, 272, 273, 274, 275, 276, 277, 278, 279, 281, 282, 284, 286, 287, 288, 289, 291, 293, 294, 295, 297, 299, 300, 302, 304, 306, 307, 308, 310, 311, 313, 315, 316, 317, 318, 319, 320, 322, 324, 326, 328, 330, 331, 333, 335, 337, 338, 340, 342, 343, 345, 346, 347, 349, 350, 351, 353, 354, 355, 357, 358, 360, 362, 363, 364, 365, 367, 369, 370, 371, 372, 373, 374, 375, 377, 378, 379, 380, 382, 383, 384, 385, 386, 387, 389, 390, 391, 393, 395, 396, 398, 400, 401, 403, 404, 406, 408, 409, 410, 411, 412, 414, 415, 417, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 456, 457, 459, 461, 462, 464, 466, 467, 468, 469, 470, 472, 474, 475, 477, 479, 480, 482, 483, 484, 485, 487, 488, 489, 490, 491, 492, 493, 494, 495, 497, 499, 501, 502, 503, 504, 506, 507, 509, 510, 511, 512, 514, 515, 516, 517, 518, 520, 522, 524, 526, 528, 530, 531, 532, 533, 534, 535, 536, 537, 538, 539, 540, 542, 543, 545, 546, 547, 548, 550, 551, 553, 554, 555, 557, 559, 560, 562, 563, 565, 566, 568, 570, 572, 573, 575, 576, 577, 579, 580, 581, 582, 583, 584, 585, 586, 587, 589, 590, 591, 592, 593, 595, 596, 597, 598, 599, 601, 602, 604, 605, 607, 609, 610, 611, 612, 613, 614, 615, 616, 617, 618, 619, 620, 621, 622, 623, 624, 625, 626, 627, 629, 631, 633, 634, 635, 36, 637, 638, 640, 642, 644, 646, 648, 650, 651, 653, 654, 655, 656, 657, 658, 659, 660, 662, 663, 664, 665, 666, 667, 669, 671, 672, 674, 676, 677, 679, 680, 682, 684, 685, 687, 689, 690, 691, 693, 694, 696, 697, 698, 00, 702, 704, 705, 706, 707, 709, 710, 712, 714, 715, 717, 718, 719, 720, 721, 722, 723, 725, 726, 727, 728, 730, 731, 732, 733, 734, 735, 737, 739, 741, 743, 744, 745, 746, 747, 749, 751, 752, 753, 755, 757, 759, 760, 762, 764, 766, 768, 770, 772, 773, 775, 776, 778, 780, 782, 784, 786, 788, 789, 791, 793, 794, 795, 796, 798, 800, 801, 802, 804, 805, 807, 809, 811, 813, 815, 817, 819, 820, 822, 823, 824, 825, 827, 828, 830, 832, 834, 836, 838, 840, 842, 843, 844, 846, 848, 849, 850, 851, 853, 855, 856, 857, 858, 860, 861, 863, 864, 866, 868, 870, 871, 873, 874, 876, 878, 880, 882, 883, 885, 887, 889, 890, 892, 893, 895, 896, 897, 899, 901, 902, 903, 905, 907, 908, 909, 910, 912, 914, 915, 916, 917, 919, 921, 923, 925, 926, 928, 930, 932, 934, 935, 936, 937, 939, 941, 943, 945, 947, 949, 950, 952, 953, 955, 957, 959, 960, 962, 964, 965, 967, 968, 970, 972, 974, 976, 978, 980, 981, 983, 985, 86, 87, 989, 991, 993, 995, 997, 998, 1000, 1002, 1003, 1005, 1007, 1008, 1009, 1011, 1013, 1014, 1016, 1018, 1020, 1022, 1023, 1024, 1025, 1026, 1027, 1029, 1031, 1033, 1034, 1036, 1038, 1039, 1041, 1042, 1043, 1044, 1045, 1046, 1048, 1049, 1050, 1051, 1052, 1054, 1056, 1058, 1060, 1061, 1062, 1064, 1066, 1067, 1069, 1070, 1071, 1072, 1074, 1076, 1077, 1078, 1079, 1081, 1083, 1085, 1087, 1088, 1090, 1092, 1093, 1095, 1096, 1097, 1099, 1101, 1102, 1104, 1105, 1107, 1108, 1110, 1111, 1112, 1113, 1114, 1115, 1117, 1119, 1120, 1121, 1122, 1124, 1126, 1128, 1130, 1132, 1133, 1135, 1137, 1139, 1141, 1142, 1144, 1146, 1148, 1150, 1152, 1154, 1156, 1157, 1158, 1160, 1162, 1164, 1166, 1168, 1169, 1171, 1173, 1175, 1176, 1177, 1179, 1180, 1181, 1183, 1185, 1187, 1188, 1190, 1192, 1193, 1194, 1196, 1198, 1200, 1201, 1203, 1205, 1207, 1209, 1210, 1212, 1214, 1216, 1218, 1219, 1221, 1222, 1224, 1226, 1228, 1230, 1232, 1234, 1235, 1236, 1238, 1240, 1241, 1243, 1245, 1247, 1249, 1251, 1252, 1253, 1255, 1257, 1259, 1261, 1263, 1265, 1266, 1268, 1270, 1272, 1274, 1276, 1278, 1279, 1281, 1283, 1285, 1287, 1289, 1291, 1293, 1294, 1296, 1297, 1299, 1300, 1302, 1304, 1306, 1308, 1309, 1310, 1312, 1313, 1314, 1316, 1318, 1320, 1322, 1324, 1325, 1326, 1328, 1329, 1330, 1332, 1333, 1334, 1336, 1338, 1339, 1340, 1341, 1342, 1343, 1344, 1345, 1347, 1349, 1350, 1352, 1353, 1355, 1357, 1358, 1359, 1360, 1361, 1363, 1364, 1366, 1367, 1369, 1370, 1371, 1372, 1373, 1375, 1376, 1377, 1379, 1380, 1381, 1382, 1384, 1386, 1388, 1389, 1391, 1393, 1395, 1397, 1399, 1401, 1402, 1403, 1405, 1407, 1409, 1410, 1412, 1414, 1416, 1418, 1420, 1421, 1422, 1424, 1426, 1428, 1429, 1431, 1433, 1435, 1437, 1439, 1440, 1441, 1443, 1445, 1446, 1448, 1450, 1452, 1454, 1456, 1457, 1459, 1461, 1463, 1465, 1467, 1469, 1471, 1473, 1475, 1477, 1479, 1481, 1482, 1483, 1485, 1486, 1488, 1489, 1490, 1492, 1493, 1495, 1497, 1498, 1500, 1502, 1503, 1505, 1506, 1507, 1508, 1509, 1511, 1512, 1514, 1515, 1516, 1518, 1520, 1522, 1523, 1524, 1525, 1526, 1528, 1529, 1531, 1533, 1534, 1535, 1537, 1538, 1540, 1542, 1544, 1546, 1547, 1549, 1550, 1552, 1554, 1555, 1557, 1558, 1560, 1562, 1563, 1564, 1565, 1568, 1569, 1571, 1572, 1574, 1575, 1577, 1578, 1580, 1581, 1583, 1584, 1586, 1588, 1589, 1591, 1593, 1594, 1595, 1596, 1598, 1599, 1600, 1602, 1603, 1604, 1605, 1607, 1609, 1611, 1612, 1613, 1615, 1616, 1618, 1619, 1621, 1622, 1624, 1626, 1628, 1629, 1630, 1632, 1634, 1636, 1637, 1639, 1640, 1642, 1644, 1646, 1647, 1648, 1649, 1650, 1652, 1654, 1656, 1657, 1658, 1660, 1661, 1662, 1664, 1666, 1668, 1669, 1670, 1671, 1672, 1674, 1676, 1677, 1679, 1680, 1681, 1682, 1683, 1684, 1686, 1688, 1689, 1691, 1693, 1695, 1697, 1699, 1700, 1701, 1702, 1704, 1705, 1706, 1707, 1708, 1710, 1711, 1712, 1713, 1714, 1715, 1717, 1719, 1721, 1723, 1724, 1725, 1727, 1729, 1731, 1732, 1734, 1735, 1737, 1739, 1741, 1743, 1745, 1746, 1747, 1749, 1751, 1753, 1755, 1757, 1758, 1759, 1761, 1762, 1764, 1765, 1766, 1767, 1769, 1770, 1772, 1773, 1774, 1776, 1777, 1779, 1781, 1783, 1784, 1786, 1788, 1790, 1792, 1794, 1795, 1797, 798, 1799, 1801, 1803, 1804, 1805, 1806, 1807, 1808, 1809, 1810, 1811, 1812, 1813, 1815, 1816, 1817, 1819, 1821, 1822, 1823, 1825, 1826, 1827, 1828, 1829, 1831, 1833, 1834, 1835, 1837, 1839, 1841, 1842, 1844, 1845, 1847, 1849, 1850, 1852, 1853, 1854, 1855, 1856, 1857, 1858, 1859, 1861, 1863, 1865, 1866, 1867, 1869, 1871, 1872, 1874, 1876, 1877, 1879, 1881, 1882, 1883, 1885, 1887, 1889, 1891, 1893, 1894, 1895, 1897, 1898, 1900, 1902, 1903, 1904, 1906, 1908, 1910, 1912, 1913, 1915, 1917, 1918, 1919, 1920, 1922, 1924, 1926, 1928, 1929, 1931, 1933, 1935, 1936, 1938, 1940, 1942, 1944, 1946, 1948, 1949, 1951, 1953, 1955, 1957, 1959, 1961, 1963, 1965, 1967, 1969, 1971, 1972, 1973, 1974, 1975, 1976, 1978, 1979, 1980, 1982, 1983, 1985, 1986, 1987, 1989, 1990, 1992, 1994, 1996, 1997, 1999, 2000, 2002, 2003, 2005, 2007, 2008, 2009, 2010, 2011, 2013, 2015, 2016, 2017, 2018, 2019, 2021, 2022, 2023, 2024, 2026, 2028, 2030, 2031, 2032, 2034, 2036, 2037, 2038, 2040, 2041, 2042, 2043, 2044, 2046, 2047, 2048, 2049, 2051, 2053, 2055, 2056, 2058, 2060, 2062, 2064, 2066, 2067, 2069, 2070, 2072, 2073, 2074, 2075, 2076, 2077, 2078, 2079, 2081, 2083, 2085, 2087, 2088, 2090, 2091, 2092, 2093, 2095, 2097, 2098, 2100, 2102, 2104, 2105, 2106, 2108, 2110, 2112, 2113, 2115, 2117, 2118, 2119, 2120, 2121, 2122, 2124, 2126, 2128, 2129, 2130, 2132, 2133, 2135, 2136, 2137, 2138, 2140, 2141, 2142, 2143, 2144, 2146, 2148, 2149, 2150, 2151, 2152, 2153, 2154, 2156, 2158, 2159, 2160, 2161, 2162, 2163, 2165, 2167, 2169, 2170, 2171, 2172, 2173, 2174, 2175, 2176, 2177, 2178, 2179, 2181, 2183, 2184, 2186, 2187, 2188, 2190, 2192, 2194, 2195, 2196, 2197, 2198, 2199, 2200, 2201, 2202, 2203, 2205, 2207, 2209, 2211, 2213, 2215, 2217, 2219, 2221, 2223, 2225, 2226, 2228, 2229, 2231, 2233, 2235, 2236, 2237, 2238, 2239, 2240, 2241, 2243, 2244, 2246, 2248, 2250, 2251, 2253, 2255, 2257, 2259, 2261, 2262, 2264, 2265, 2267, 2269, 2270, 2272, 2273, 2275, 2276, 2278, 2279, 2281, 2282, 2284, 2285, 2286, 2288, 2289, 2291, 2293, 2295, 2296, 2297, 2298, 2299, 2300, 2302, 2304, 2306, 2307, 2309, 2310, 2312, 2313, 2314, 2315, 2316, 2317, 2318, 2320, 2321, 2323, 2325, 2327, 2329, 2330, 2332, 2334, 2335, 2337, 2339, 2341, 2343, 2345, 2346, 2347, 2348, 2350, 2352, 2354, 2356, 2357, 2358, 2360, 2361, 2362, 2364, 2366, 2367, 2369, 2371, 2373, 2375, 2376, 2378, 2380, 2381, 2383, 2384, 2386, 2388, 2390, 2391, 2392, 2394, 2396, 2397, 2399, 2401, 2402, 2403, 2405, 2407, 2408, 2409, 2411, 2413, 2415, 2417, 2418, 2419, 2421, 2422, 2423, 2424, 2425, 2427, 2428, 2429, 2430, 2432, 2434, 2435, 2437, 2439, 2440, 2441, 2442, 2443, 2445, 2446, 2448, 2449, 2451, 2452, 2454, 2456, 2457, 2458, 2459, 2460, 2461, 2462, 2463, 2464, 2466, 2468, 2469, 2470, 2471, 2472, 2474, 2475, 2476, 2477, 2478, 2480, 2482, 2483, 2484, 2485, 2486, 2487, 2488, 2489, 2490, 2491, 2493, 2494, 2495, 2496, 2497, 2498, 2499, 2500, 2502, 2503, 2505, 2506, 2507, 2509, 2510, 2511, 2512, 2513, 2514, 2516, 2517, 2518, 2519, 2521, 2522, 2523, 2525, 2527, 2529, 2530, 2531, 2533, 2535, 2537, 2539, 2540, 2541, 2542, 2544, 2546, 2547, 2548, 2550, 2551, 2552, 2554, 2556, 2557, 2558, 2559, 2561, 2562, 2564, 2565, 2567, 2569, 2571, 2572, 2573, 2574, 2575, 2576, 2578, 2580, 2581, 2583, 2584, 2585, 2587, 2588, 2589, 2590, 2592, 2593, 2594, 2595, 2597, 2599, 2601, 2603, 2604, 2606, 2608, 2609, 2611, 2612, 2614, 2615, 2616, 2617, 2619, 2621, 2623, 2624, 2626, 2627, 2628, 2629, 2631, 2632, 2634, 2636, 2638, 2640, 2641, 2642, 2644, 2646, 2647, 2648, 2649, 2650, 2652, 2654, 2656, 2658, 2659, 2661, 2663, 2665, 2667, 2668, 2670, 2672, 2674, 2675, 2677, 2679, 2681, 2683, 2685, 2687, 2689, 2691, 2693, 2695, 2697, 2699, 2701, 2703, 2704, 2706, 2708, 2710, 2711, 2712, 2714, 2716, 2717, 2719, 2721, 2722, 2724, 2726, 2728, 2730, 2732, 2734, 2735, 2736, 2738, 2739, 2740, 2741, 2743, 2745, 2746, 2748, 2750, 2751, 2753, 2754, 2756, 2758, 2760, 2762, 2764, 2766, 2768, 2770, 2771, 2772, 2773, 2775, 2777, 2778, 2780, 2781, 2782, 2784, 2786, 2788, 2790, 2791, 2792, 2794, 2796, 2797, 2799, 2801, 2802, 2803, 2804, 2806, 2807, 2809, 2810, 2811, 2813, 2815, 2816, 2818, 2819, 2821, 2822, 2823, 2825, 2826, 2828, 2830, 2832, 2833, 2835, 2836, 2838, 2840, 2842, 2844, 2845, 2846, 2847, 2849, 2850, 2851, 2852, 2853, 2855, 2856, 2857, 2858, 2859, 2861, 2862, 2863, 2864, 2866, 2867, 2868, 2869, 2870, 2872, 2873, 2875, 2876, 2877, 2878, 2880, 2882, 2883, 2885, 2886, 2888, 2890, 2891, 2892, 2893, 2894, 2896, 2898, 2900, 2902, 2903, 2904, 2906, 2907, 2908, 2909, 2910, 2911, 2915, 2917, 2918, 2919, 2920, 2921, 2922, 2923, 2924 or 2926, wherein a plant produced from said plant cell has a difference in the level of biomass as compared to the corresponding level of a control plant that does not comprise said nucleic acid.

3.-63. (canceled)

64. A plant cell comprising an exogenous nucleic acid, said exogenous nucleic acid comprising a regulatory region operably linked to a nucleotide sequence encoding a polypeptide, wherein the HMM bit score of the amino acid sequence of said polypeptide is greater than about 93, said HMM based on the amino acid sequences depicted in one of FIGS. 1-50, and wherein said plant has a difference in the level of biomass as compared to the corresponding level of a control plant that does not comprise said nucleic acid.

65. A plant cell comprising an exogenous nucleic acid said exogenous nucleic acid comprising a regulatory region operably linked to a nucleotide sequence encoding a polypeptide having 80 percent or greater sequence identity to an amino acid sequence selected from the group consisting of SEQ ID NO:2, 4, 5, 6, 8, 10, 12, 13, 15, 17, 18, 20, 22, 24, 25, 26, 27, 29, 30, 32, 34, 35, 37, 38, 39, 41, 43, 45, 47, 49, 51, 53, 55, 56, 57, 59, 60, 62, 63, 64, 65, 67, 69, 70, 71, 73, 74, 75, 77, 79, 80, 81, 82, 83, 84, 85, 86, 88, 90, 91, 92, 93, 94, 95, 96, 97, 98, 99, 100, 101, 102, 103, 104, 105, 106, 107, 108, 109, 110, 111, 112, 113, 115, 117, 119, 120, 121, 122, 123, 124, 126, 128, 129, 131, 133, 135, 137, 139, 140, 141, 142, 144, 146, 147, 148, 149, 150, 152, 154, 155, 157, 158, 160, 162, 163, 165, 167, 169, 171, 173, 175, 176, 178, 180, 182, 183, 185, 187, 189, 191, 193, 194, 195, 196, 197, 198, 199, 200, 201, 203, 205, 207, 208, 209, 211, 213, 214, 215, 217, 218, 219, 220, 222, 223, 224, 226, 228, 229, 231, 232, 233, 234, 235, 236, 237, 238, 239, 240, 242, 244, 245, 246, 247, 248, 249, 250, 251, 252, 254, 255, 256, 257, 258, 259, 260, 261, 262, 263, 264, 265, 266, 267, 268, 269, 270, 271, 272, 273, 274, 275, 276, 277, 278, 279, 281, 282, 284, 286, 287, 288, 289, 291, 293, 294, 295, 297, 299, 300, 302, 304, 306, 307, 308, 310, 311, 313, 315, 316, 317, 318, 319, 320, 322, 324, 326, 328, 330, 331, 333, 335, 337, 338, 340, 342, 343, 345, 346, 347, 349, 350, 351, 353, 354, 355, 357, 358, 360, 362, 363, 364, 365, 367, 369, 370, 371, 372, 373, 374, 375, 377, 378, 379, 380, 382, 383, 384, 385, 386, 387, 389, 390, 391, 393, 395, 396, 398, 400, 401, 403, 404, 406, 408, 409, 410, 411, 412, 414, 415, 417, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 456, 457, 459, 461, 462, 464, 466, 467, 468, 469, 470, 472, 474, 475, 477, 479, 480, 482, 483, 484, 485, 487, 488, 489, 490, 491, 492, 493, 494, 495, 497, 499, 501, 502, 503, 504, 506, 507, 509, 510, 511, 512, 514, 515, 516, 517, 518, 520, 522, 524, 526, 528, 530, 531, 532, 533, 534, 535, 536, 537, 538, 539, 540, 542, 543, 545, 546, 547, 548, 550, 551, 553, 554, 555, 557, 559, 560, 562, 563, 565, 566, 568, 570, 572, 573, 575, 576, 577, 579, 580, 581, 582, 583, 584, 585, 586, 587, 589, 590, 591, 592, 593, 595, 596, 597, 598, 599, 601, 602, 604, 605, 607, 609, 610, 611, 612, 613, 614, 615, 616, 617, 618, 619, 620, 621, 622, 623, 624, 625, 626, 627, 629, 631, 633, 634, 635, 36, 637, 638, 640, 642, 644, 646, 648, 650, 651, 653, 654, 655, 656, 657, 658, 659, 660, 662, 663, 664, 665, 666, 667, 669, 671, 672, 674, 676, 677, 679, 680, 682, 684, 685, 687, 689, 690, 691, 693, 694, 696, 697, 698, 00, 702, 704, 705, 706, 707, 709, 710, 712, 714, 715, 717, 718, 719, 720, 721, 722, 723, 725, 726, 727, 728, 730, 731, 732, 733, 734, 735, 737, 739, 741, 743, 744, 745, 746, 747, 749, 751, 752, 753, 755, 757, 759, 760, 762, 764, 766, 768, 770, 772, 773, 775, 776, 778, 780, 782, 784, 786, 788, 789, 791, 793, 794, 795, 796, 798, 800, 801, 802, 804, 805, 807, 809, 811, 813, 815, 817, 819, 820, 822, 823, 824, 825, 827, 828, 830, 832, 834, 836, 838, 840, 842, 843, 844, 846, 848, 849, 850, 851, 853, 855, 856, 857, 858, 860, 861, 863, 864, 866, 868, 870, 871, 873, 874, 876, 878, 880, 882, 883, 885, 887, 889, 890, 892, 893, 895, 896, 897, 899, 901, 902, 903, 905, 907, 908, 909, 910, 912, 914, 915, 916, 917, 919, 921, 923, 925, 926, 928, 930, 932, 934, 935, 936, 937, 939, 941, 943, 945, 947, 949, 950, 952, 953, 955, 957, 959, 960, 962, 964, 965, 967, 968, 970, 972, 974, 976, 978, 980, 981, 983, 985, 86, 87, 989, 991, 993, 995, 997, 998, 1000, 1002, 1003, 1005, 1007, 1008, 1009, 1011, 1013, 1014, 1016, 1018, 1020, 1022, 1023, 1024, 1025, 1026, 1027, 1029, 1031, 1033, 1034, 1036, 1038, 1039, 1041, 1042, 1043, 1044, 1045, 1046, 1048, 1049, 1050, 1051, 1052, 1054, 1056, 1058, 1060, 1061, 1062, 1064, 1066, 1067, 1069, 1070, 1071, 1072, 1074, 1076, 1077, 1078, 1079, 1081, 1083, 1085, 1087, 1088, 1090, 1092, 1093, 1095, 1096, 1097, 1099, 1101, 1102, 1104, 1105, 1107, 1108, 1110, 1111, 1112, 1113, 1114, 1115, 1117, 1119, 1120, 1121, 1122, 1124, 1126, 1128, 1130, 1132, 1133, 1135, 1137, 1139, 1141, 1142, 1144, 1146, 1148, 1150, 1152, 1154, 1156, 1157, 1158, 1160, 1162, 1164, 1166, 1168, 1169, 1171, 1173, 1175, 1176, 1177, 1179, 1180, 1181, 1183, 1185, 1187, 1188, 1190, 1192, 1193, 1194, 1196, 1198, 1200, 1201, 1203, 1205, 1207, 1209, 1210, 1212, 1214, 1216, 1218, 1219, 1221, 1222, 1224, 1226, 1228, 1230, 1232, 1234, 1235, 1236, 1238, 1240, 1241, 1243, 1245, 1247, 1249, 1251, 1252, 1253, 1255, 1257, 1259, 1261, 1263, 1265, 1266, 1268, 1270, 1272, 1274, 1276, 1278, 1279, 1281, 1283, 1285, 1287, 1289, 1291, 1293, 1294, 1296, 1297, 1299, 1300, 1302, 1304, 1306, 1308, 1309, 1310, 1312, 1313, 1314, 1316, 1318, 1320, 1322, 1324, 1325, 1326, 1328, 1329, 1330, 1332, 1333, 1334, 1336, 1338, 1339, 1340, 1341, 1342, 1343, 1344, 1345, 1347, 1349, 1350, 1352, 1353, 1355, 1357, 1358, 1359, 1360, 1361, 1363, 1364, 1366, 1367, 1369, 1370, 1371, 1372, 1373, 1375, 1376, 1377, 1379, 1380, 1381, 1382, 1384, 1386, 1388, 1389, 1391, 1393, 1395, 1397, 1399, 1401, 1402, 1403, 1405, 1407, 1409, 1410, 1412, 1414, 1416, 1418, 1420, 1421, 1422, 1424, 1426, 1428, 1429, 1431, 1433, 1435, 1437, 1439, 1440, 1441, 1443, 1445, 1446, 1448, 1450, 1452, 1454, 1456, 1457, 1459, 1461, 1463, 1465, 1467, 1469, 1471, 1473, 1475, 1477, 1479, 1481, 1482, 1483, 1485, 1486, 1488, 1489, 1490, 1492, 1493, 1495, 1497, 1498, 1500, 1502, 1503, 1505, 1506, 1507, 1508, 1509, 1511, 1512, 1514, 1515, 1516, 1518, 1520, 1522, 1523, 1524, 1525, 1526, 1528, 1529, 1531, 1533, 1534, 1535, 1537, 1538, 1540, 1542, 1544, 1546, 1547, 1549, 1550, 1552, 1554, 1555, 1557, 1558, 1560, 1562, 1563, 1564, 1565, 1568, 1569, 1571, 1572, 1574, 1575, 1577, 1578, 1580, 1581, 1583, 1584, 1586, 1588, 1589, 1591, 1593, 1594, 1595, 1596, 1598, 1599, 1600, 1602, 1603, 1604, 1605, 1607, 1609, 1611, 1612, 1613, 1615, 1616, 1618, 1619, 1621, 1622, 1624, 1626, 1628, 1629, 1630, 1632, 1634, 1636, 1637, 1639, 1640, 1642, 1644, 1646, 1647, 1648, 1649, 1650, 1652, 1654, 1656, 1657, 1658, 1660, 1661, 1662, 1664, 1666, 1668, 1669, 1670, 1671, 1672, 1674, 1676, 1677, 1679, 1680, 1681, 1682, 1683, 1684, 1686, 1688, 1689, 1691, 1693, 1695, 1697, 1699, 1700, 1701, 1702, 1704, 1705, 1706, 1707, 1708, 1710, 1711, 1712, 1713, 1714, 1715, 1717, 1719, 1721, 1723, 1724, 1725, 1727, 1729, 1731, 1732, 1734, 1735, 1737, 1739, 1741, 1743, 1745, 1746, 1747, 1749, 1751, 1753, 1755, 1757, 1758, 1759, 1761, 1762, 1764, 1765, 1766, 1767, 1769, 1770, 1772, 1773, 1774, 1776, 1777, 1779, 1781, 1783, 1784, 1786, 1788, 1790, 1792, 1794, 1795, 1797, 798, 1799, 1801, 1803, 1804, 1805, 1806, 1807, 1808, 1809, 1810, 1811, 1812, 1813, 1815, 1816, 1817, 1819, 1821, 1822, 1823, 1825, 1826, 1827, 1828, 1829, 1831, 1833, 1834, 1835, 1837, 1839, 1841, 1842, 1844, 1845, 1847, 1849, 1850, 1852, 1853, 1854, 1855, 1856, 1857, 1858, 1859, 1861, 1863, 1865, 1866, 1867, 1869, 1871, 1872, 1874, 1876, 1877, 1879, 1881, 1882, 1883, 1885, 1887, 1889, 1891, 1893, 1894, 1895, 1897, 1898, 1900, 1902, 1903, 1904, 1906, 1908, 1910, 1912, 1913, 1915, 1917, 1918, 1919, 1920, 1922, 1924, 1926, 1928, 1929, 1931, 1933, 1935, 1936, 1938, 1940, 1942, 1944, 1946, 1948, 1949, 1951, 1953, 1955, 1957, 1959, 1961, 1963, 1965, 1967, 1969, 1971, 1972, 1973, 1974, 1975, 1976, 1978, 1979, 1980, 1982, 1983, 1985, 1986, 1987, 1989, 1990, 1992, 1994, 1996, 1997, 1999, 2000, 2002, 2003, 2005, 2007, 2008, 2009, 2010, 2011, 2013, 2015, 2016, 2017, 2018, 2019, 2021, 2022, 2023, 2024, 2026, 2028, 2030, 2031, 2032, 2034, 2036, 2037, 2038, 2040, 2041, 2042, 2043, 2044, 2046, 2047, 2048, 2049, 2051, 2053, 2055, 2056, 2058, 2060, 2062, 2064, 2066, 2067, 2069, 2070, 2072, 2073, 2074, 2075, 2076, 2077, 2078, 2079, 2081, 2083, 2085, 2087, 2088, 2090, 2091, 2092, 2093, 2095, 2097, 2098, 2100, 2102, 2104, 2105, 2106, 2108, 2110, 2112, 2113, 2115, 2117, 2118, 2119, 2120, 2121, 2122, 2124, 2126, 2128, 2129, 2130, 2132, 2133, 2135, 2136, 2137, 2138, 2140, 2141, 2142, 2143, 2144, 2146, 2148, 2149, 2150, 2151, 2152, 2153, 2154, 2156, 2158, 2159, 2160, 2161, 2162, 2163, 2165, 2167, 2169, 2170, 2171, 2172, 2173, 2174, 2175, 2176, 2177, 2178, 2179, 2181, 2183, 2184, 2186, 2187, 2188, 2190, 2192, 2194, 2195, 2196, 2197, 2198, 2199, 2200, 2201, 2202, 2203, 2205, 2207, 2209, 2211, 2213, 2215, 2217, 2219, 2221, 2223, 2225, 2226, 2228, 2229, 2231, 2233, 2235, 2236, 2237, 2238, 2239, 2240, 2241, 2243, 2244, 2246, 2248, 2250, 2251, 2253, 2255, 2257, 2259, 2261, 2262, 2264, 2265, 2267, 2269, 2270, 2272, 2273, 2275, 2276, 2278, 2279, 2281, 2282, 2284, 2285, 2286, 2288, 2289, 2291, 2293, 2295, 2296, 2297, 2298, 2299, 2300, 2302, 2304, 2306, 2307, 2309, 2310, 2312, 2313, 2314, 2315, 2316, 2317, 2318, 2320, 2321, 2323, 2325, 2327, 2329, 2330, 2332, 2334, 2335, 2337, 2339, 2341, 2343, 2345, 2346, 2347, 2348, 2350, 2352, 2354, 2356, 2357, 2358, 2360, 2361, 2362, 2364, 2366, 2367, 2369, 2371, 2373, 2375, 2376, 2378, 2380, 2381, 2383, 2384, 2386, 2388, 2390, 2391, 2392, 2394, 2396, 2397, 2399, 2401, 2402, 2403, 2405, 2407, 2408, 2409, 2411, 2413, 2415, 2417, 2418, 2419, 2421, 2422, 2423, 2424, 2425, 2427, 2428, 2429, 2430, 2432, 2434, 2435, 2437, 2439, 2440, 2441, 2442, 2443, 2445, 2446, 2448, 2449, 2451, 2452, 2454, 2456, 2457, 2458, 2459, 2460, 2461, 2462, 2463, 2464, 2466, 2468, 2469, 2470, 2471, 2472, 2474, 2475, 2476, 2477, 2478, 2480, 2482, 2483, 2484, 2485, 2486, 2487, 2488, 2489, 2490, 2491, 2493, 2494, 2495, 2496, 2497, 2498, 2499, 2500, 2502, 2503, 2505, 2506, 2507, 2509, 2510, 2511, 2512, 2513, 2514, 2516, 2517, 2518, 2519, 2521, 2522, 2523, 2525, 2527, 2529, 2530, 2531, 2533, 2535, 2537, 2539, 2540, 2541, 2542, 2544, 2546, 2547, 2548, 2550, 2551, 2552, 2554, 2556, 2557, 2558, 2559, 2561, 2562, 2564, 2565, 2567, 2569, 2571, 2572, 2573, 2574, 2575, 2576, 2578, 2580, 2581, 2583, 2584, 2585, 2587, 2588, 2589, 2590, 2592, 2593, 2594, 2595, 2597, 2599, 2601, 2603, 2604, 2606, 2608, 2609, 2611, 2612, 2614, 2615, 2616, 2617, 2619, 2621, 2623, 2624, 2626, 2627, 2628, 2629, 2631, 2632, 2634, 2636, 2638, 2640, 2641, 2642, 2644, 2646, 2647, 2648, 2649, 2650, 2652, 2654, 2656, 2658, 2659, 2661, 2663, 2665, 2667, 2668, 2670, 2672, 2674, 2675, 2677, 2679, 2681, 2683, 2685, 2687, 2689, 2691, 2693, 2695, 2697, 2699, 2701, 2703, 2704, 2706, 2708, 2710, 2711, 2712, 2714, 2716, 2717, 2719, 2721, 2722, 2724, 2726, 2728, 2730, 2732, 2734, 2735, 2736, 2738, 2739, 2740, 2741, 2743, 2745, 2746, 2748, 2750, 2751, 2753, 2754, 2756, 2758, 2760, 2762, 2764, 2766, 2768, 2770, 2771, 2772, 2773, 2775, 2777, 2778, 2780, 2781, 2782, 2784, 2786, 2788, 2790, 2791, 2792, 2794, 2796, 2797, 2799, 2801, 2802, 2803, 2804, 2806, 2807, 2809, 2810, 2811, 2813, 2815, 2816, 2818, 2819, 2821, 2822, 2823, 2825, 2826, 2828, 2830, 2832, 2833, 2835, 2836, 2838, 2840, 2842, 2844, 2845, 2846, 2847, 2849, 2850, 2851, 2852, 2853, 2855, 2856, 2857, 2858, 2859, 2861, 2862, 2863, 2864, 2866, 2867, 2868, 2869, 2870, 2872, 2873, 2875, 2876, 2877, 2878, 2880, 2882, 2883, 2885, 2886, 2888, 2890, 2891, 2892, 2893, 2894, 2896, 2898, 2900, 2902, 2903, 2904, 2906, 2907, 2908, 2909, 2910, 2911, 2915, 2917, 2918, 2919, 2920, 2921, 2922, 2923, 2924 and 2926, wherein a plant produced from said plant cell has a difference in the level of biomass as compared to the corresponding level of a control plant that does not comprise said nucleic acid.

66. (canceled)

67. A transgenic plant comprising the plant cell of claim 64.

68. The transgenic plant of claim 67, wherein said plant further exhibits an increase or decrease in lignin content of about 2 percent to 100 percent as compared to a control plant that does not express said exogenous nucleic acid, wherein said difference in lignin content is.

69. The transgenic plant of claim 68, wherein said increase in lignin content is a 2 percent to 85 percent increase and wherein said difference in the level of biomass is an increase in the level of biomass of about 17 percent to 100 percent.

70. The transgenic plant of claim 68, wherein said increase in lignin content is a 5 percent to 40 percent increase and wherein said plant further exhibits an increase in glucan content of 7 percent to 20 percent as compared to a control plant that does not express said exogenous nucleic acid.

71. The transgenic plant of claim 70, wherein said increase in lignin content is a 14 percent to 30 percent increase, said increase in glucan content is 7 percent to 20 percent and said difference in the level of biomass is an increase in biomass of 46 percent to 92 percent.

72. The transgenic plant of claim 68, wherein said decrease in lignin content is a 2 percent to 53 percent decrease and said difference in the level of biomass is a 14 percent to 120 percent increase.

73. The transgenic plant of claim 68, wherein said decrease in lignin content is a 3 percent to 40 percent decrease and said plant further exhibits a 19 percent to 53 percent increase glucan content as compared to a control plant that does not express said exogenous nucleic acid.

74. The transgenic plant of claim 67, wherein said plant further exhibits decreased ash content as compared to a control plant that does not express said exogenous nucleic acid.

75. The transgenic plant of claim 67, wherein said plant is a member of a species selected from the group consisting of Panicum virgatum (switchgrass), Sorghum bicolor (sorghum, sudangrass), Miscanthus giganteus (miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza sativa (rice), Helianthus annuus (sunflower), Medicago sativa (alfalfa), Beta vulgaris (sugarbeet), or Pennisetum glaucum (pearl millet).

76.-84. (canceled)

85. A transgenic plant comprising the plant cell of claim 65.

86. The transgenic plant of claim 85, wherein said difference in the composition of biomass comprises a difference in the level of a biomass component selected from the group consisting of lignin, glucan, S:G ratio, and ash, as compared to the corresponding level of the biomass composition of a control plant that does not comprise said nucleic acid.

87. The transgenic plant of claim 86, wherein said composition of biomass comprises increased lignin content of about 2 percent to 100 percent.

88. The transgenic plant of claim 87, wherein said lignin content is increased 5 percent to 40 percent and wherein said composition of biomass further comprises an increase in glucan content of 7 percent to 20 percent.

89. The transgenic plant of claim 86, wherein said composition of biomass comprises decreased lignin content of about 2 percent to 100 percent.

90. The transgenic plant of claim 89, wherein said lignin content is decreased 3 percent to 40 percent and said composition of biomass further comprises an increase in glucan content of 19 percent to 53 percent.

91. The transgenic plant of claim 86, wherein said composition of biomass comprises decreased ash content.

92. The transgenic plant of claim 86, wherein said composition of biomass comprises increased glucan content of 10 percent to 20 percent.

93. The transgenic plant of claim 85, wherein said plant is a member of a species selected from the group consisting of Panicum virgatum (switchgrass), Sorghum bicolor (sorghum, sudangrass), Miscanthus giganteus (miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza sativa (rice), Helianthus annuus (sunflower), Medicago sativa (alfalfa), Beta vulgaris (sugarbeet), or Pennisetum glaucum (pearl millet).

94.-103. (canceled)
